(19) United States
(12) Patent Application Publication
(10) Pub. No.: US 2024/0317777 A1
(43) Pub. Date: Sep. 26, 2024

(54) NOVEL MACROCYCLIC AMINOPYRAZOLE COMPOUNDS AS CDK2 INHIBITORS

(71) Applicant: Accutar Biotechnology Inc., Cranbury, NJ (US)

(72) Inventors: Xiangzhu Wang, Branford, CT (US); Jie SU, New York, NY (US); Ke LIU, Shanghai (CN); Wei HE, Zionsville, IN (US); Jie FAN, New York, NY (US); Yimin QIAN, Plainsboro, NJ (US)

(73) Assignee: Accutar Biotechnology Inc., Cranbury, NJ (US)

(21) Appl. No.: 18/586,171

(22) Filed: Feb. 23, 2024

Related U.S. Application Data
(60) Provisional application No. 63/486,534, filed on Feb. 23, 2023.

(51) Int. Cl. C07D 498/18; A61K 31/439; A61K 31/4748; A61K 31/4995; A61K 31/504; A61K 31/529; A61P 35/00; C07D 498/22; C07D 513/22; C07D 515/18

(52) U.S. Cl. CPC C07D 498/18; A61K 31/439; A61K 31/4748; A61K 31/4995; A61K 31/504; A61K 31/529; A61P 35/00; C07D 498/22; C07D 513/22; C07D 515/18

(57) ABSTRACT

The present disclosure relates to a compound of Formula (I), a tautomer, a stereoisomer or a mixture of stereoisomers, a pharmaceutically acceptable salt, a hydrate, or a deuterated derivative thereof and their use in, e.g., treating a disease or disorder associated with CDK2. The present disclosure also relates to pharmaceutical compositions containing such compounds, and their use in treating or preventing a disease or disorder associated with CDK2.

This application claims the benefit of priority to U.S. Provisional Patent Application No. 63/486,534, filed Feb. 23, 2023, which is hereby incorporated by reference in its entirety.

The present disclosure relates to novel macrocyclic aminopyrazole compounds, pharmaceutical compositions containing such compounds, and their use in prevention and treatment of diseases and conditions, e.g., cancer. The compounds disclosed herein exhibit selective CDK2 inhibition activity.

Cyclin-Dependent Kinases (CDKs) are a family of protein kinases that participate in regulating a wide range of physiological processes. For example, CDKs have been identified to contribute to regulating cell cycle, mRNA processing, differentiation of nerve cells, and other biological functions. Most of the known CDK complexes, however, regulate progression through the cell cycle and are present in all known eukaryotes. CDKs are also implicated in the development and proliferation of cancer cells via unregulated and abnormal expression of CDKs (see, e.g., “Cell cycle, CDKs and cancer: a changing paradigm, Nature Review Cancer, 2009, 9, 153-166; and “Inhibiting CDK in Cancer Therapy: Current Evidence and Future Directions”, Target Oncology, 2018, 13(1), 21-38).

Cyclin-dependent kinase 2 (CDK2) is a CDK enzyme that has been implicated in human cancers. A binding partner to CDK2 is cyclin E, which is produced by the cyclin E1 (CCNE1) gene, and upon binding the activated CDK2 regulates the transition from G1 to S phase in DNA replication. Overactivation of the CCNE1 gene, due to a mutation, has been reported to influence cancer cell proliferation through the CDK2 pathway (see, e.g., “Mechanism of Cdk2/Cyclin E inhibition of p27 and p27 phosphorylation”, Biochemistry, 1999, 38(27), 8713-8722). Cancers that have been identified to form because of a CCNE1 mutation include ovarian, gastric, endometrial, and breast (see, e.g., “Cyclin E and survival in patients with breast cancer”, N Engl J Med, 2002, 347(20), 1566-75). Therapy approaches targeting CDK2 have induced cell cycle arrest and tumor growth inhibition, yet selective inhibition of CDK2 was not definitively concluded (see, e.g., “Selective killing of transformed cells by cyclin/cyclin-dependent kinase 2 antagonists”, PNAS, 1999, 96(8), 4325-4329). The close similarities between CDK2 and other CDK binding sites may cause unintentional inhibition of other CDK-mediated pathways and thus result in serious side-effects. Moreover, despite significant research efforts, there are currently no approved agents targeting CDK2 (see, e.g., “Highlights of the Latest Advances in Research on CDK Inhibitors”, Cancers, 2014, 6(4), 2224-2242).

Thus, there is an unmet medical need for novel compounds, such as CDK inhibitors that have novel activity profiles and selectively target CDK2.

The present disclosure provides, according to some embodiments, compounds, compositions, and methods for modulating the activity of CDK2. In some embodiments, the compounds exhibit selective CDK2 inhibition activity.

In some embodiments, the present disclosure provides a compound represented by Formula (I), or a tautomer, a stereoisomer or a mixture of stereoisomers, a pharmaceutically acceptable salt, a hydrate, or a deuterated derivative thereof:

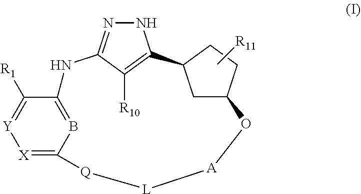

wherein:
A is a substituted or unsubstituted amide or a 5-12 membered aromatic ring, wherein the 5-12 membered aromatic ring optionally includes one or more heteroatoms and is optionally substituted with 1, 2, 3, or 4 R_{A1}, wherein each R_{A1} is independently selected from hydrogen, —C(═O)R_{A2}, C₁-C₅alkyl, C₂-C₅ alkenyl, C₂-C₅ alkynyl, halo, C₁-C₅ haloalkyl, C₁-C₅alkoxy, and —CN, and wherein each R_{A2} is independently selected from hydrogen, hydroxyl, C₁-C₅alkyl, C₁-C₅alkoxy, C₁-C₅haloalkyl, C₂-C₅ alkenyl, and C₂-C₅ alkynyl;
B is selected from N, N⁺—O⁻, and CR₀, wherein R₀ is selected from hydrogen, halo, —CN, C₁-C₅alkyl, C₁-C₅haloalkyl, C₁-C₅alkoxy, and C₃-C₅ cycloalkyl;
R₁ is selected from hydrogen, halo, —CN, and C₁-C₅ alkyl, or R₁ and Y together with the carbon atom to which they are attached form a 5-6 membered aromatic ring, wherein the aromatic ring optionally includes 1 to 3 heteroatoms;
X and Y are independently N, N⁺—O⁻, or CR₂, wherein each R₂ is independently selected from hydrogen, haloalkyl, C₁-C₅ alkyl, C₁-C₅ alkoxy, and C₃-C₅ cycloalkyl;
Q is absent or selected from —W—C(R₃)(R₄)—, wherein R₃ and R₄ are independently selected from hydrogen, deuterium, C₁-C₅ alkyl, C₁-C₅ haloalkyl, and halo, or R₃ and R₄ together with the carbon atom to which they are attached form a carbonyl group or a 3-6 membered cycloalkyl, and wherein W is absent or selected from alkylene, —C(═O)—R₆—, —O—, —R₆—O—, —O—R₆—, —N(R₇)—, —NH—C(═O)—, —C(═O)—N(R₆)—, —S—, —S(═O)₂—, —P(R₇)₂—, 3-6 membered cycloalkyl, and 3-6 membered heterocycle, wherein the alkylene, 3-6 membered cycloalkyl, and 3-6 membered heterocycle are optionally substituted with 1, 2, 3, 4, or 5 R_{B}, wherein each R₆ is independently selected from C₁-C₅ alkylene, 3-6 membered heterocycle, and 3-6 membered cycloalkyl, wherein each R₇ is independently selected from hydrogen, oxo, and C₁-C₅ alkyl, and wherein each R_{B} is independently selected from hydrogen, oxo, halo, and C₁-C₅ alkyl;
L is absent or a linker of 1 to 10 carbon atoms in length, wherein one or more carbon atoms are optionally substituted with oxo, C₁-C₅ haloalkyl, halo, deuterium, or C₁-C₅ alkyl, and wherein one or more carbon atoms are optionally replaced by a 3-6-membered bridged cycloalkyl, a 3-7 membered fused cycloalkyl, alkenyl, —O—, —N(R₈)—, a 3-6-membered cycloalkyl, or a 3-6-membered heterocycle, wherein the 3-6-membered heterocycle, the 3-6-membered cycloalkyl, and the 3-6-membered bridged cycloalkyl are optionally substituted with 1, 2, 3, or 4 R₉, wherein each R₈ is independently selected from hydrogen and C₁-C₅ alkyl, and wherein each R₉ is independently selected from hydrogen, oxo, and C₁-C₅ alkyl; and
R₁₀ and R₁₁ are independently selected from hydrogen, halo, and C₁-C₅ alkyl; with the proviso that the compound of Formula (I) is not the following:
(1¹S,1³R,Z)-2¹H-12-oxa-3,6,10-triaza-4(4,2)-pyridina-2(3,5)-pyrazola-1(1,3)-cyclopentanacyclododecaphane-5,11-dione,
(1¹S,1³R,Z)-2¹H-12-oxa-3,6,10-triaza-4(3,5)-pyridina-2(5,3)-pyrazola-1(1,3)-cyclopentanacyclododecaphane-5,11-dione,
(1¹S,1³R,Z)-2¹H-12-oxa-3,6,10-triaza-2(5,3)-pyrazola-4(1,3)-benzena-1(1,3)-cyclopentanacyclododecaphane-5,11-dione, or
(1¹S,1³R,Z)-2¹H-13-oxa-3,6,11-triaza-2(5,3)-pyrazola-4(1,3)-benzena-1(1,3)-cyclopentanacyclotridecaphane-5,12-dione.

In some embodiments, disclosed is a compound of Formula (I) selected from compounds of Formula (II):

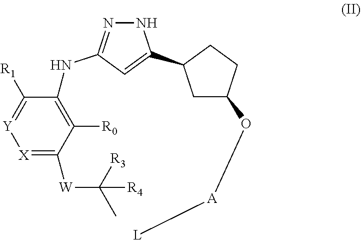

wherein R₀, R₁, X, Y, W, R₃, R₄, L, and A are as defined above.

In some embodiments, disclosed is a compound of Formula (I) selected from compounds of Formula (III):

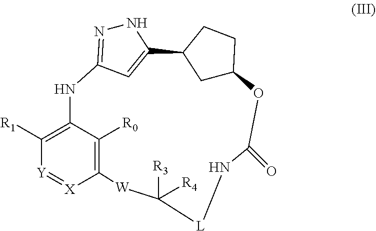

wherein R₀, R₁, X, Y, W, R₃, R₄, and L are as defined above.

In some embodiments, disclosed is a compound of Formula (I) selected from compounds of Formula (IV):

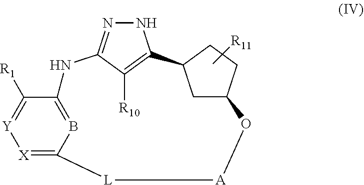

wherein B, R₁, X, Y, L, A, R₁₀, and R₁₁ are as defined above.

In some embodiments, disclosed is a compound of Formula (I) selected from compounds of Formula (V):

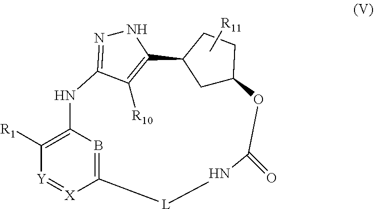

wherein R₁, B, X, Y, R₁₀, R₁₁, and L are as defined above.

Also disclosed herein is a method of treating a disease or disorder, in a subject in need thereof, comprising administering to said subject at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, or a pharmaceutical composition comprising at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, and at least one pharmaceutically acceptable carrier or excipient. In some embodiments, the pharmaceutical composition of the present disclosure may be for use in (or in the manufacture of medicaments for) the treatment of the disease or disorder in the subject in need thereof.

In some embodiments, a therapeutically effective amount of a pharmaceutical composition of the present disclosure may be administered to a subject diagnosed with the disease or disorder. In some embodiments, the disease or disorder is cancer. In some embodiments, the cancer is selected from breast cancer, ovarian cancer, uterine cancer, endometrial cancer, cervical cancer, bladder cancer, prostate cancer, lung cancer, stomach cancer, esophageal cancer, colorectal cancer, small bowel cancer, pancreatic cancer, liver cancer, kidney cancer, head and neck cancer, skin cancer, bone cancer, thyroid cancer, peritoneal cancer, and brain cancer.

### BRIEF DESCRIPTION OF THE DRAWINGS

FIG. 1 is a diagram illustrating general synthesis method 1.

FIG. 2 is a diagram illustrating general synthesis method 2.

FIG. 3 is a diagram illustrating general synthesis method 3.

FIG. 4 is a diagram illustrating the synthesis of compound 16.

FIG. 5 is a diagram illustrating the synthesis of compound 4.

FIG. 6 is a diagram illustrating the synthesis of compound 8, compound 9, and compound 10.

FIG. 7 is a diagram illustrating the synthesis of compound 9.

FIG. 8 is a diagram illustrating the synthesis of compound 13.

FIG. 9 is a diagram illustrating the synthesis of compound 17.

FIG. 10 is a diagram illustrating the synthesis of compound 18.

FIG. 11 is a diagram illustrating the synthesis of compound 22.

FIG. 12 is a diagram illustrating the synthesis of compound 26.

FIG. 13 is a diagram illustrating the synthesis of compound 33.

FIG. 14 is a diagram illustrating the synthesis of compound 104.

FIG. 15 is a diagram illustrating the synthesis of compound 21.

FIG. 16 is a diagram illustrating the synthesis of compound 20.

FIG. 17 is a diagram illustrating the synthesis of compound 23.

FIG. 18 is a diagram illustrating the synthesis of compound 109 and compound 110.

FIG. 19 is a diagram illustrating the synthesis of compound 11 and compound 12.

FIG. 20 is a diagram illustrating the synthesis of compound 24 and compound 25.

FIG. 21 is a diagram illustrating the synthesis of compound 71 and compound 72.

FIG. 22 is a diagram illustrating the synthesis of compound 108.

FIG. 23 is a diagram illustrating the synthesis of compound 112 and compound 113.

FIG. 24 is a diagram illustrating the synthesis of compound 114.

FIG. 25 is a diagram illustrating the synthesis of compound 115.

FIG. 26 is a diagram illustrating the synthesis of compound 125.

FIG. 27 is a diagram illustrating the synthesis of compound 126.

FIG. 28 is a diagram illustrating the synthesis of compound 127.

FIG. 29 is a diagram illustrating the synthesis of compound 138.

FIG. 30 is a diagram illustrating the synthesis of compound 139 and compound 140.

FIG. 31 is a diagram illustrating the synthesis of compound 146.

FIG. 32 is a diagram illustrating the synthesis of compound 158.

FIG. 33 is a diagram illustrating the synthesis of compound 159.

FIG. 34 is a diagram illustrating the synthesis of compound 162.

FIG. 35 is a diagram illustrating the synthesis of compound 163.

FIG. 36 is a diagram illustrating the synthesis of compound 164 and compound 165.

FIG. 37 is a diagram illustrating the synthesis of compound 166.

FIG. 38 is a diagram illustrating the synthesis of compound 167, compound 168, compound 169, and compound 170.

FIG. 39 is a diagram illustrating the synthesis of compound 173.

FIG. 40 is a diagram illustrating the synthesis of compound 174.

FIG. 41 is a diagram illustrating the synthesis of compound 178.

FIG. 42 is a diagram illustrating the synthesis of compound 181.

FIG. 43 is a diagram illustrating the synthesis of compound 182.

FIG. 44 is a diagram illustrating the synthesis of compound 183.

FIG. 45 is a diagram illustrating the synthesis of compound 184.

FIG. 46 is a diagram illustrating the synthesis of compound 188.

FIG. 47 is a diagram illustrating the synthesis of compound 219.

FIG. 48 is a diagram illustrating the synthesis of compound 223.

FIG. 49 is a diagram illustrating the synthesis of compound 224 and compound 225.

FIG. 50 is a diagram illustrating the synthesis of compound 229 and compound 230.

FIG. 51 is a diagram illustrating the synthesis of compound 231 and compound 232.

FIG. 52 is a diagram illustrating the synthesis of compound 235.

FIG. 53 is a diagram illustrating the synthesis of compound 236.

FIG. 54 is a diagram illustrating the synthesis of compound 237 and compound 238.

FIG. 55 is a diagram illustrating the synthesis of compound 241.

FIG. 56 is a diagram illustrating the synthesis of compound 256.

FIG. 57 is a diagram illustrating the synthesis of compound 226 and compound 227.

FIG. 58 is a diagram illustrating the synthesis of compound 239 and compound 240.

FIG. 59 is a diagram illustrating the synthesis of compound 257 and compound 258.

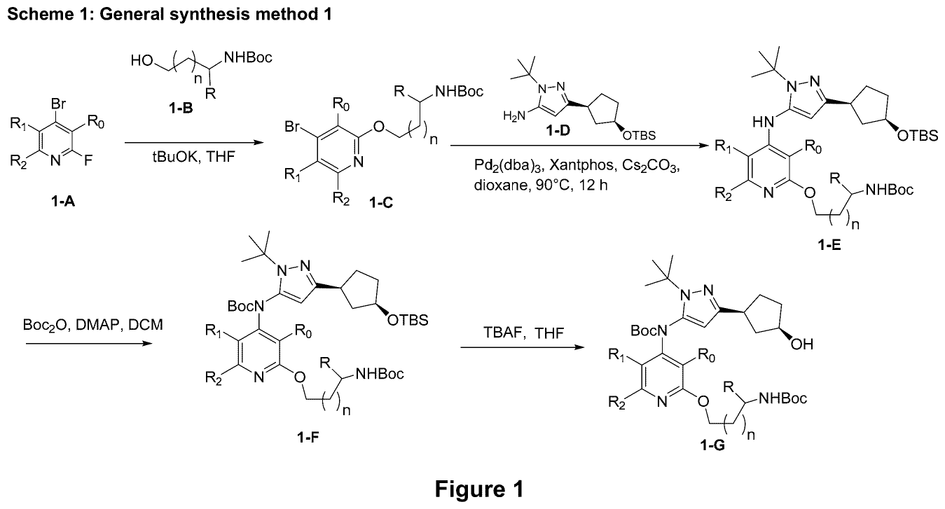

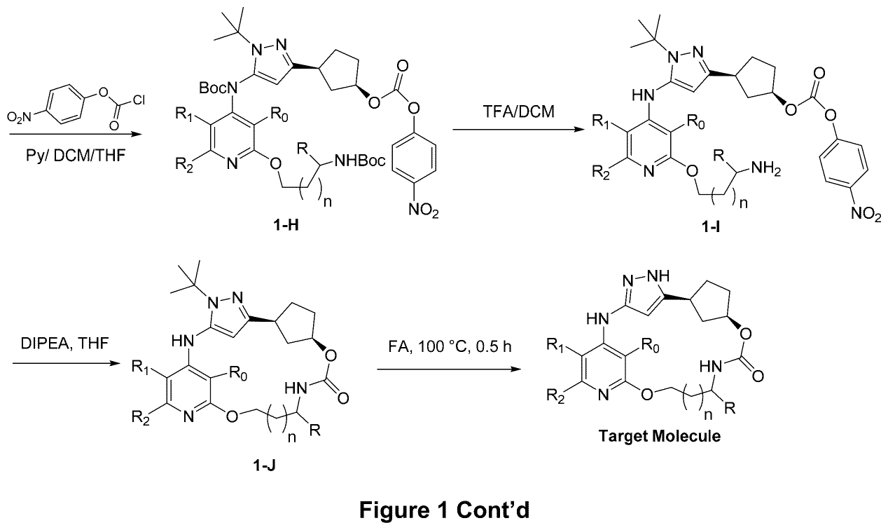

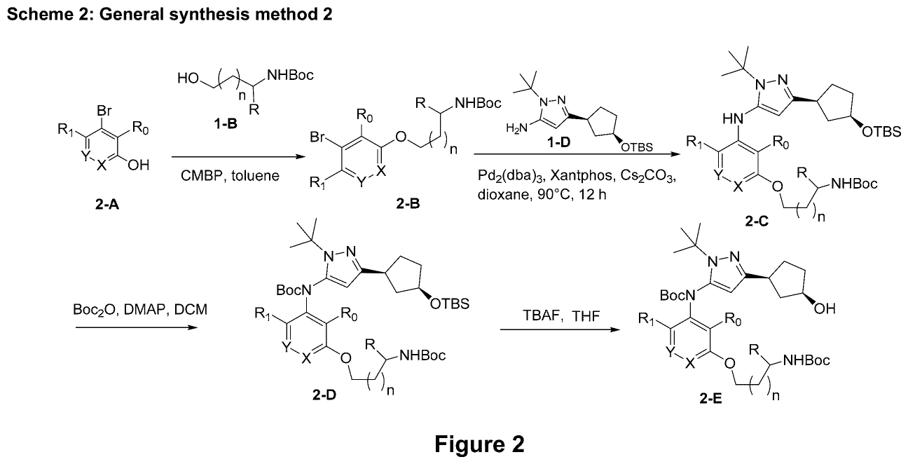

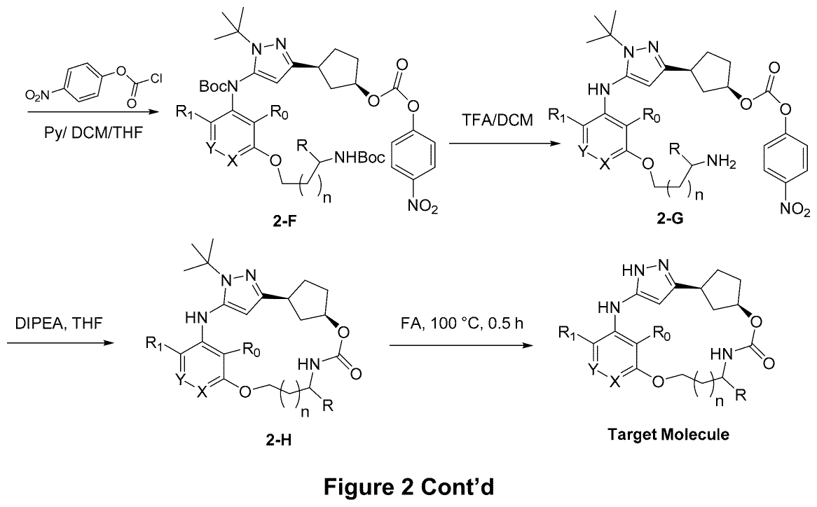

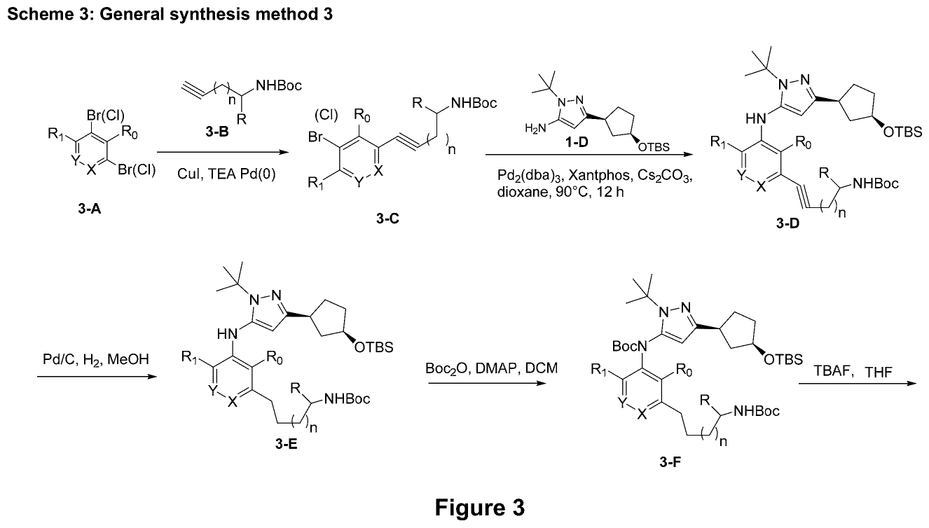

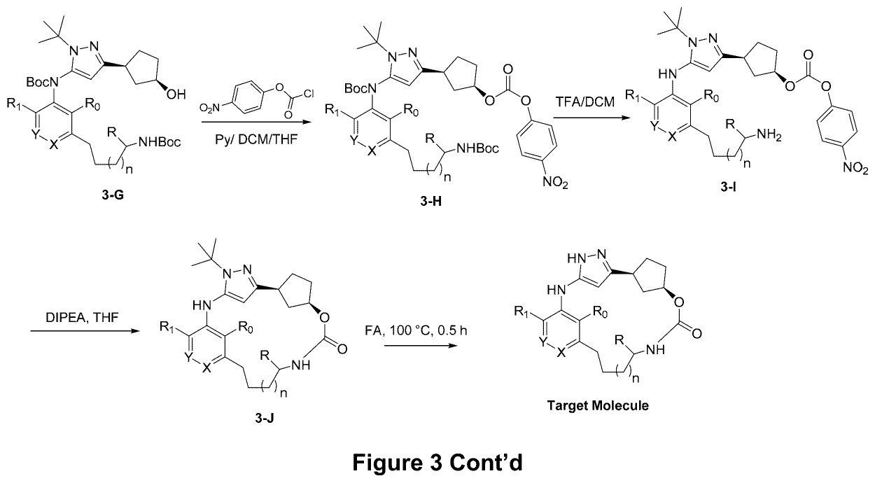

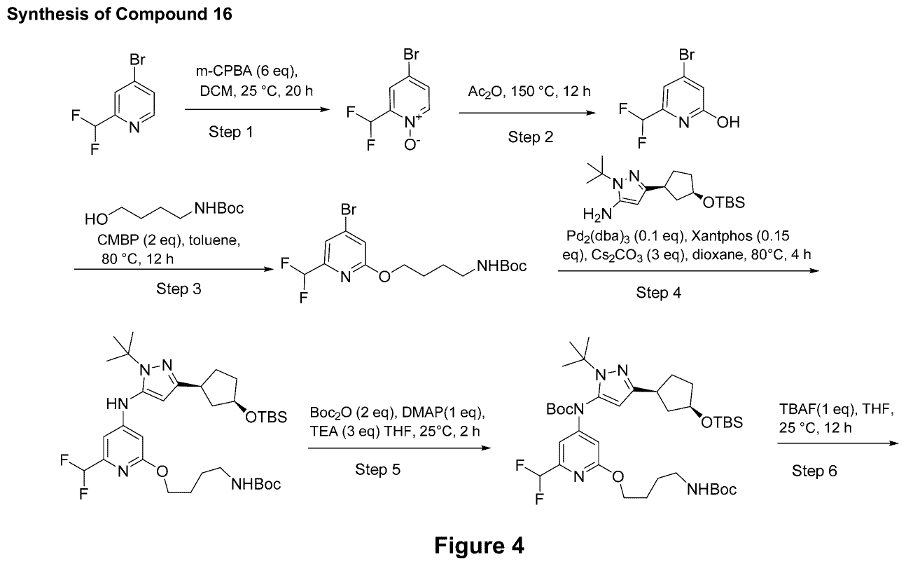

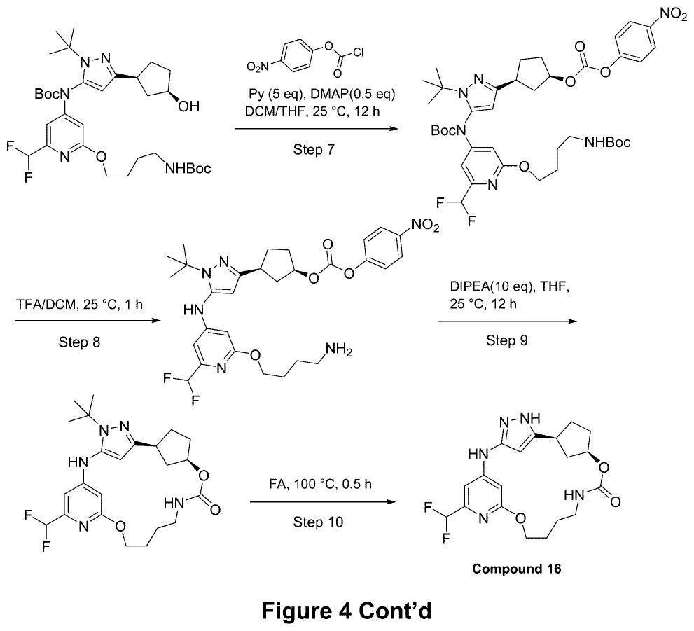

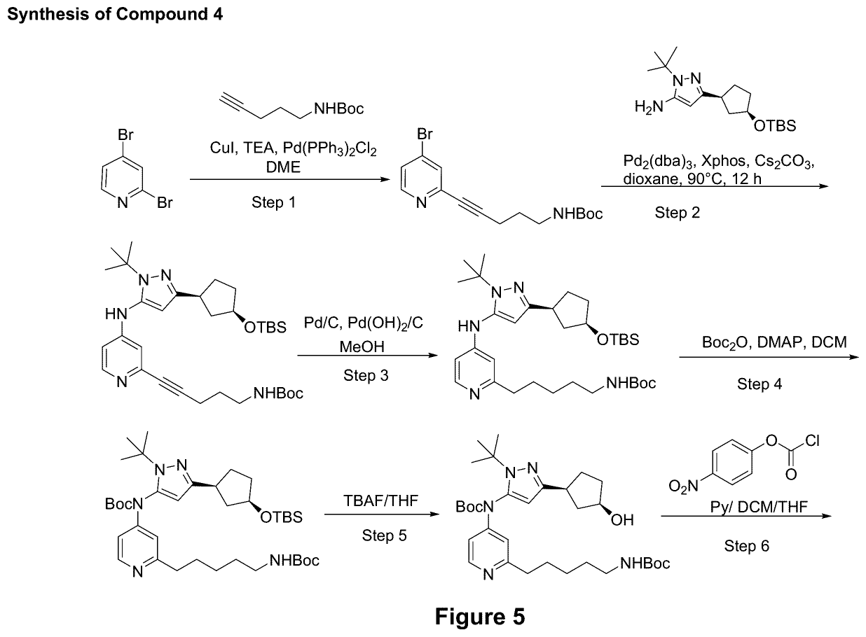

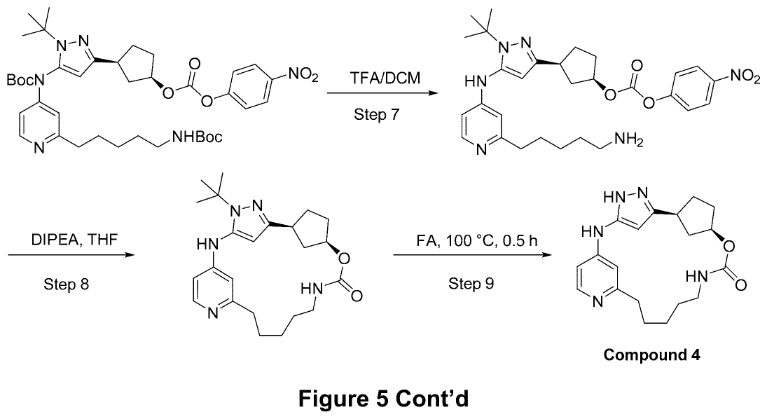

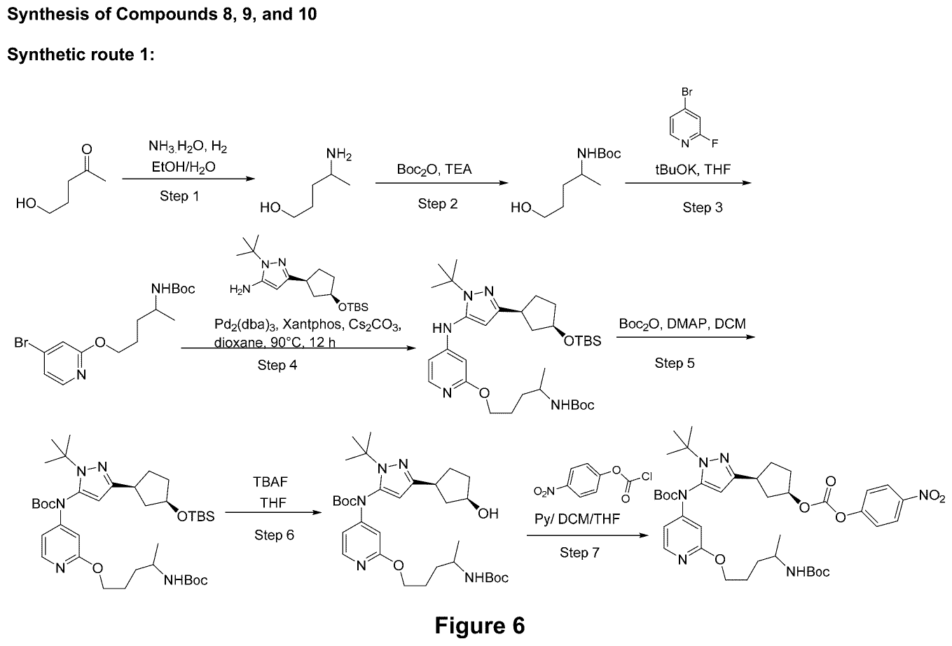

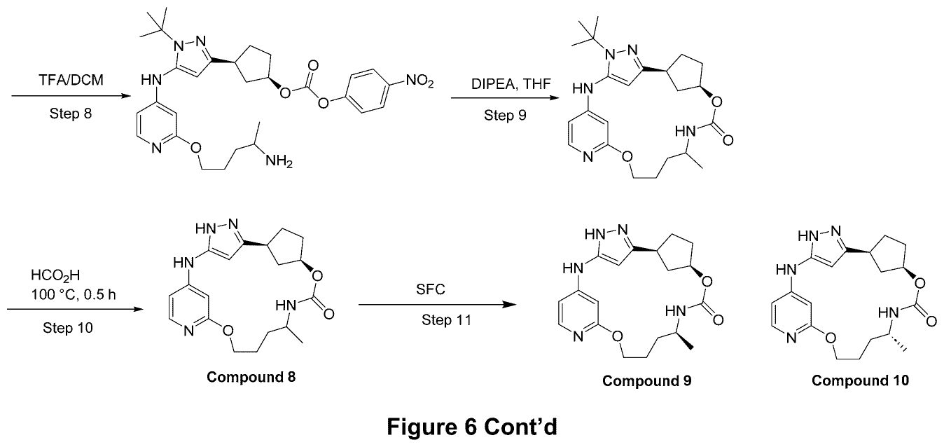

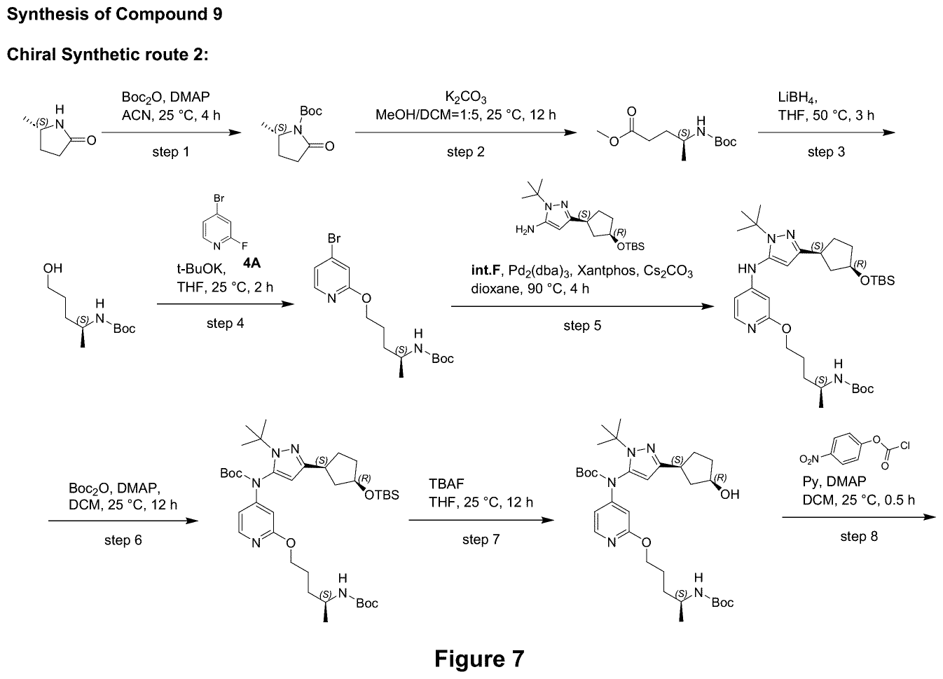

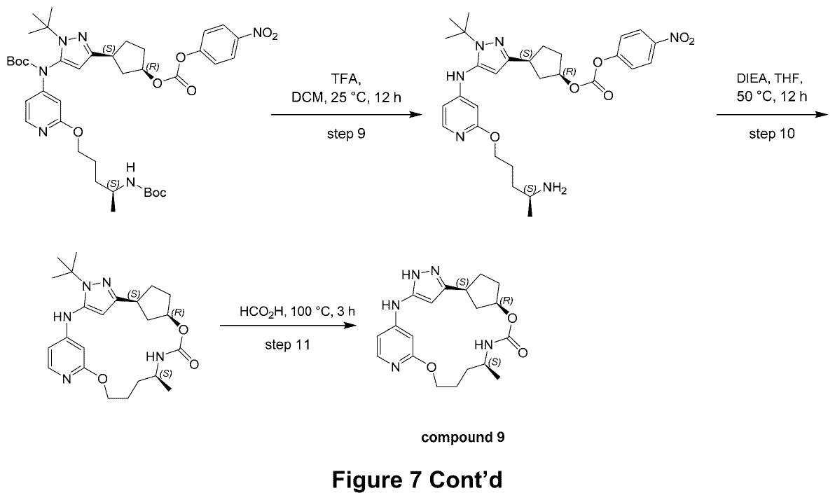

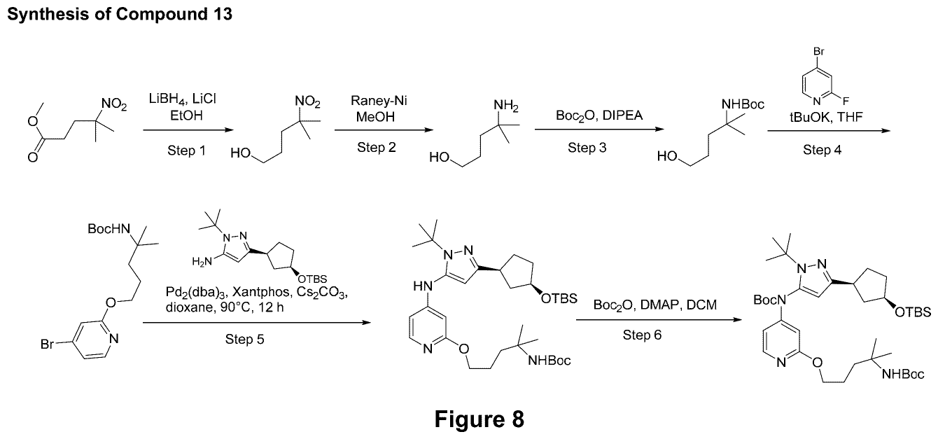

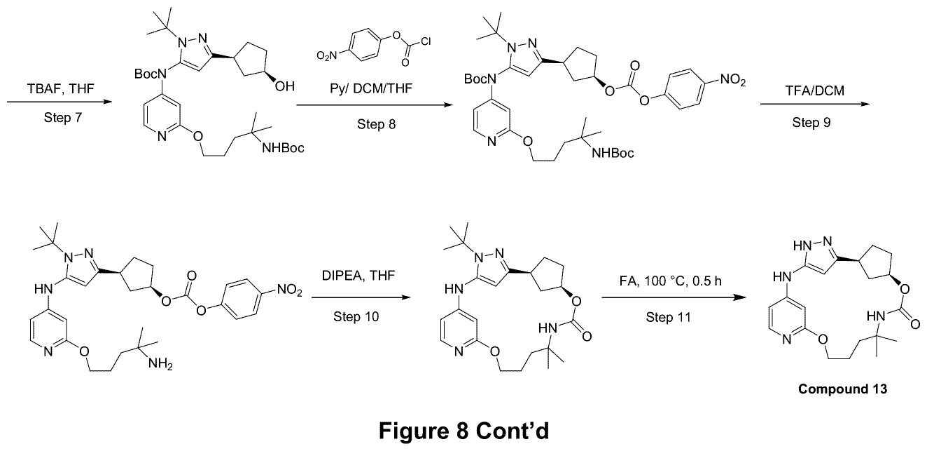

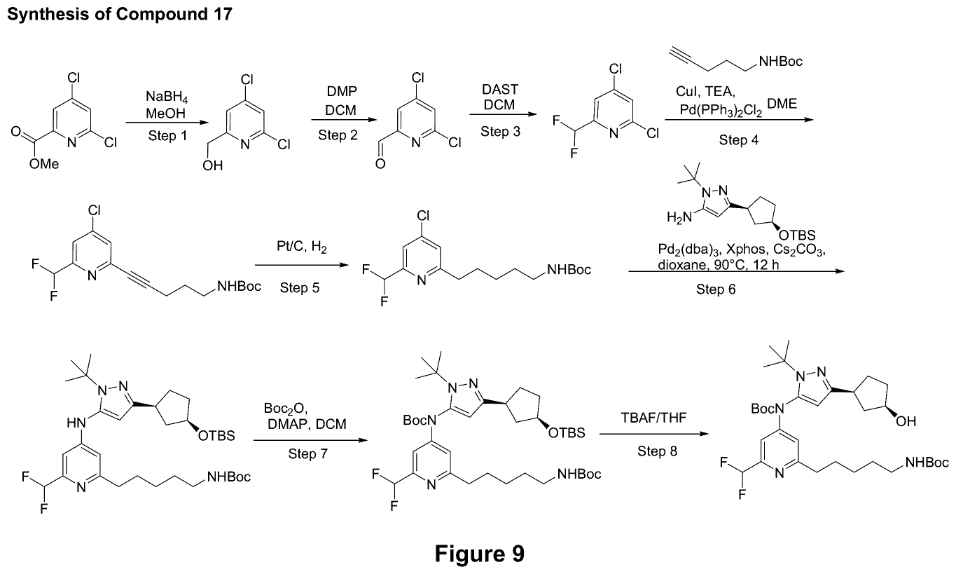

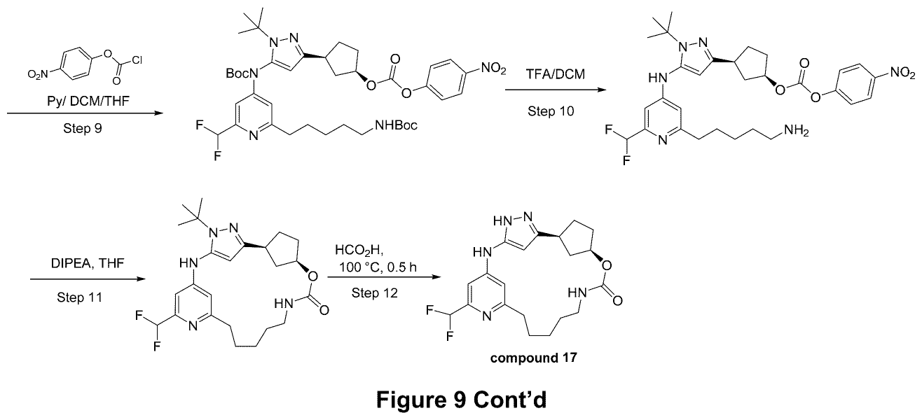

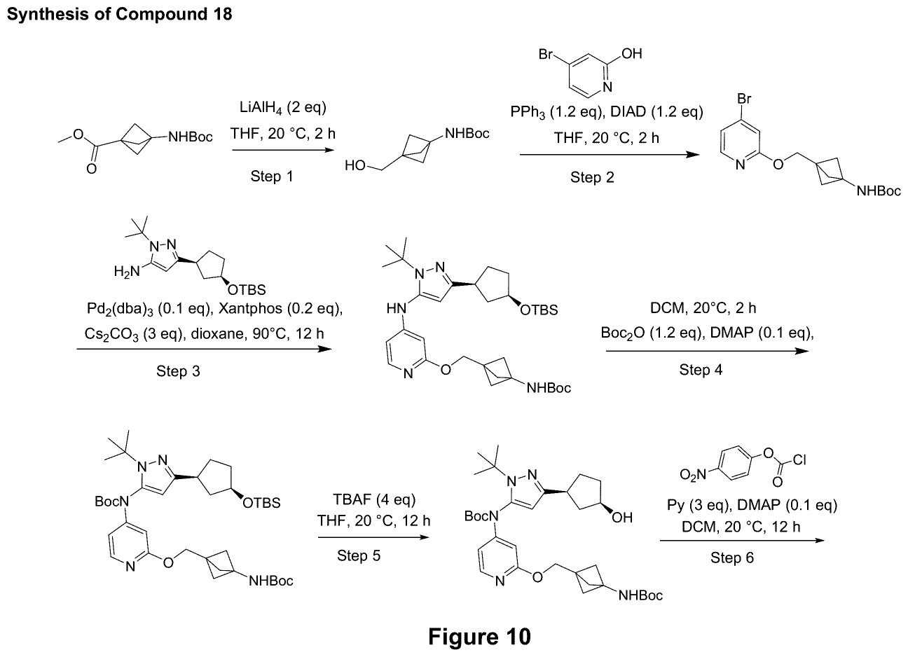

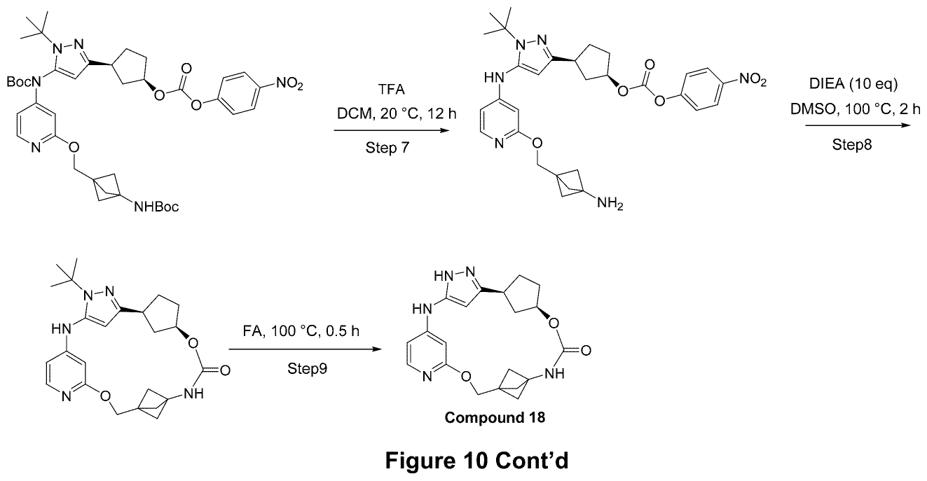

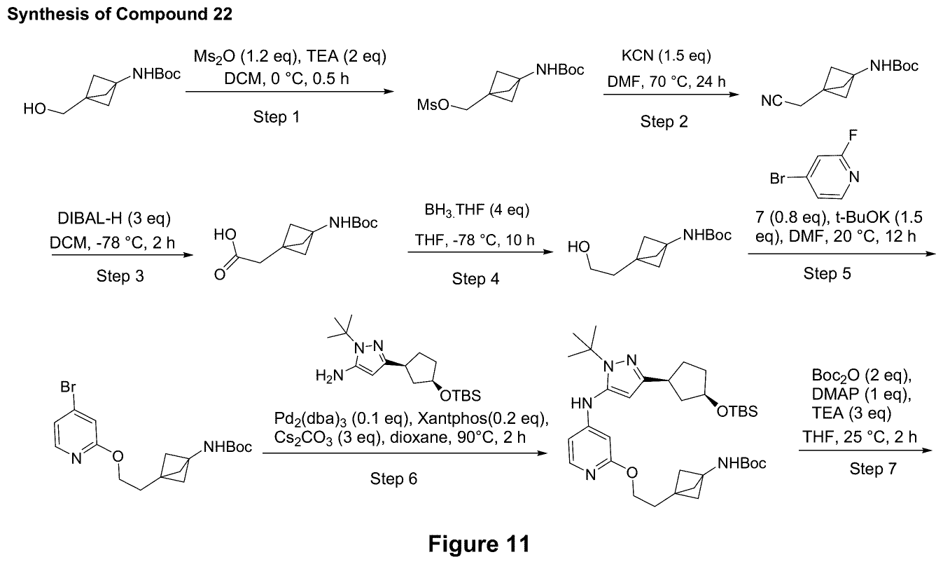

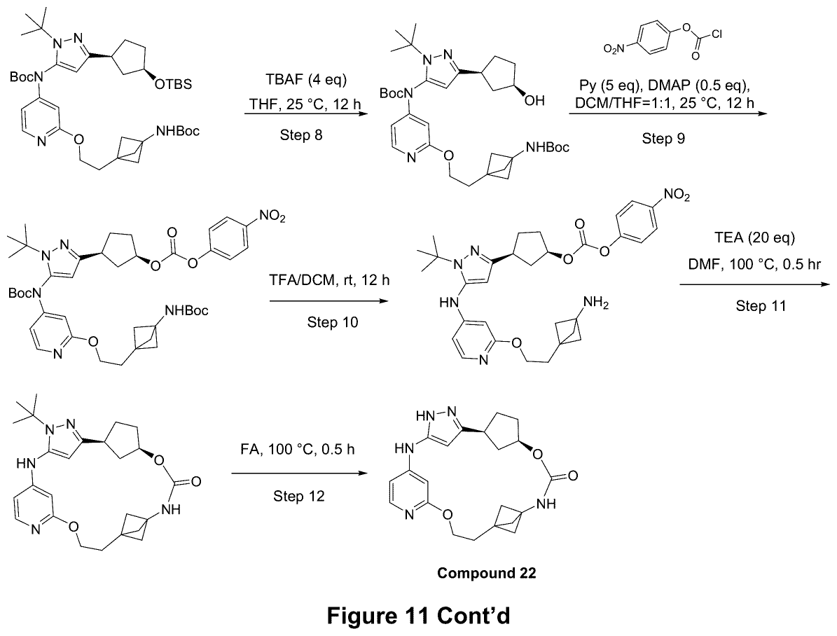

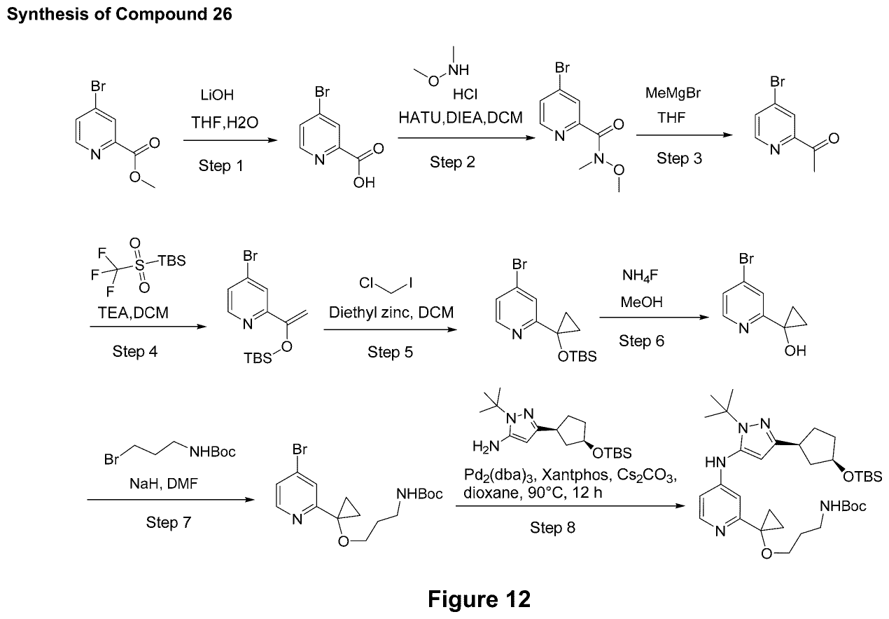

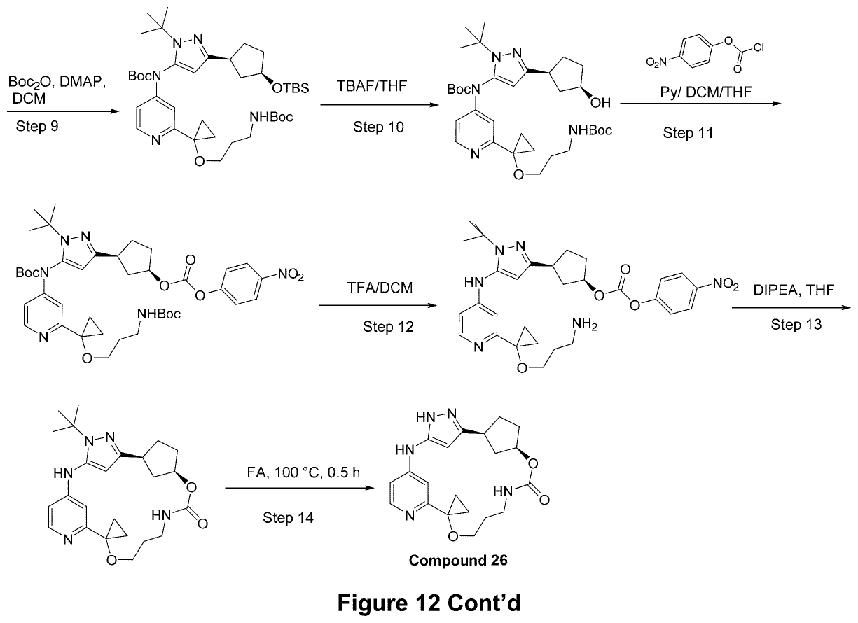

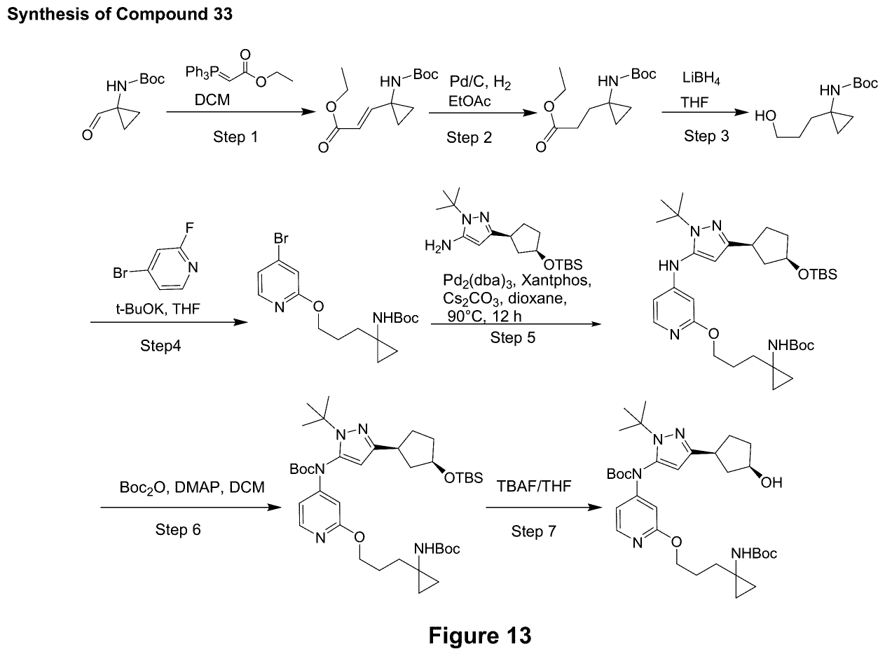

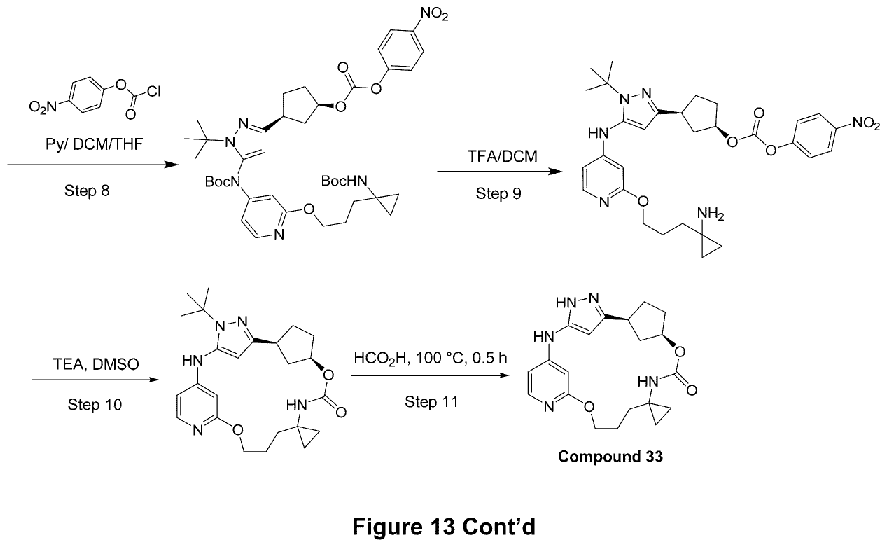

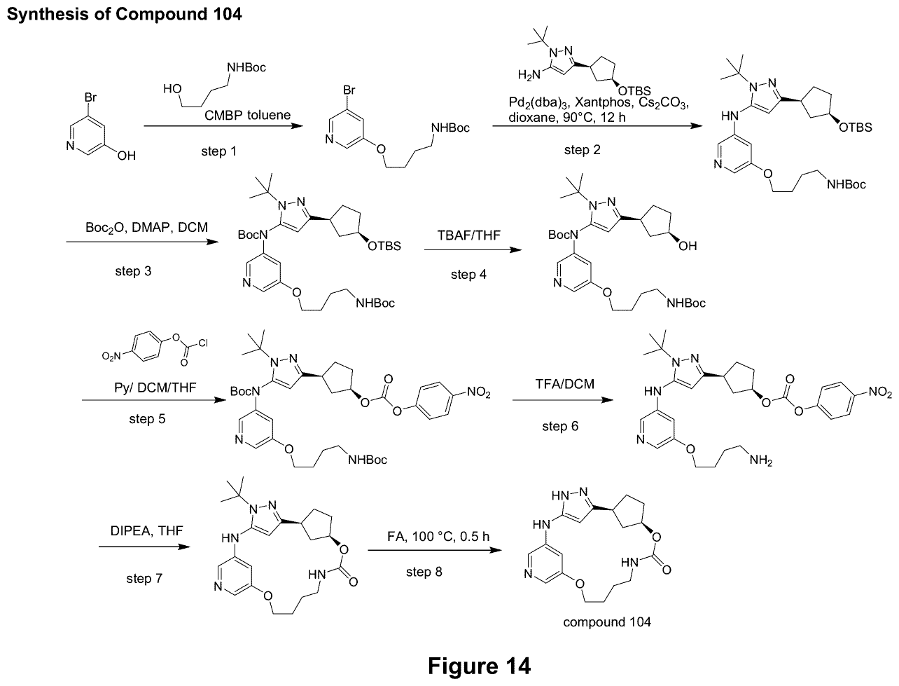

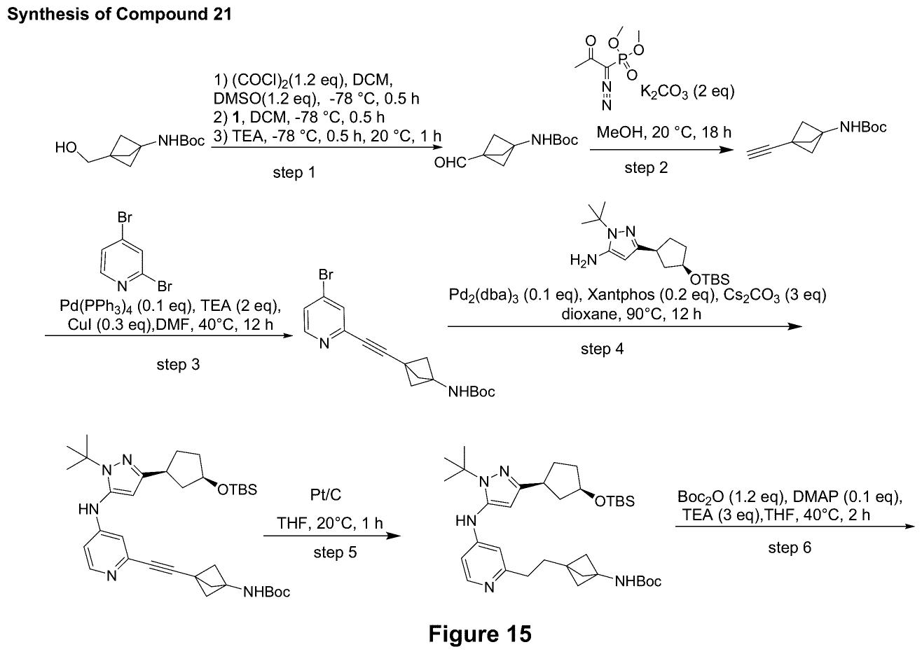

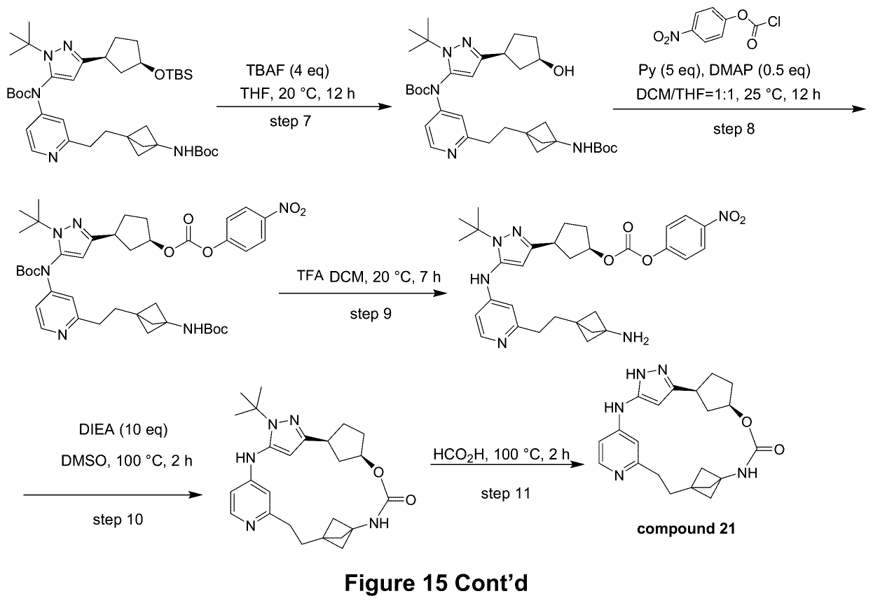

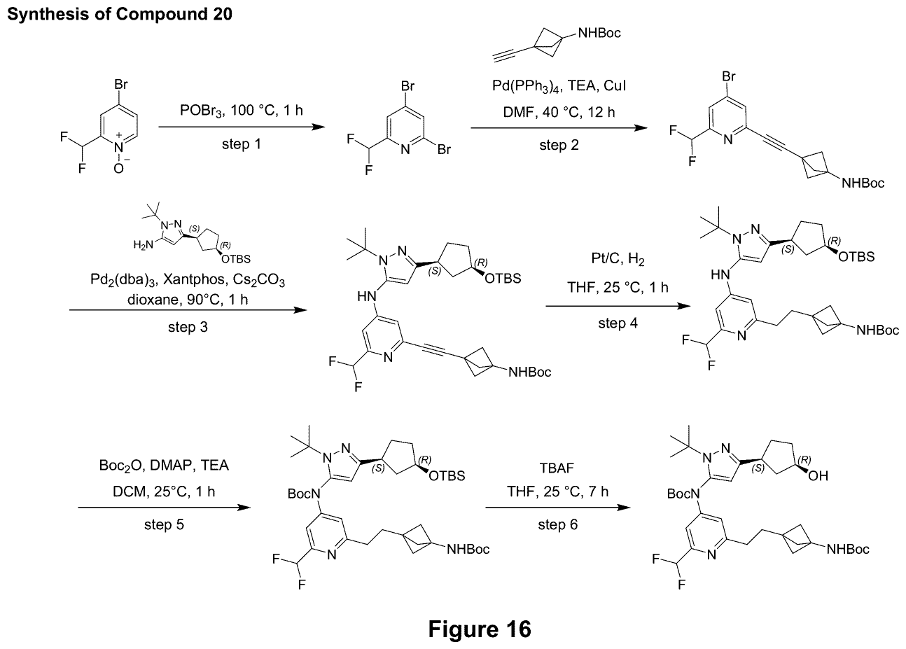

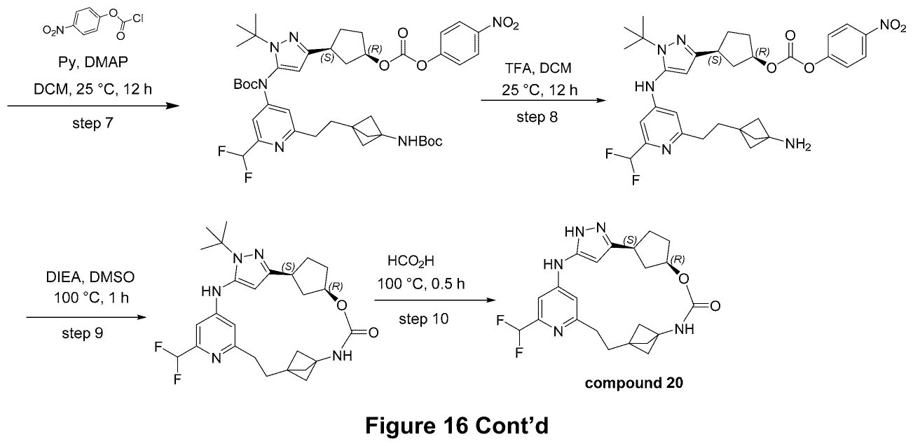

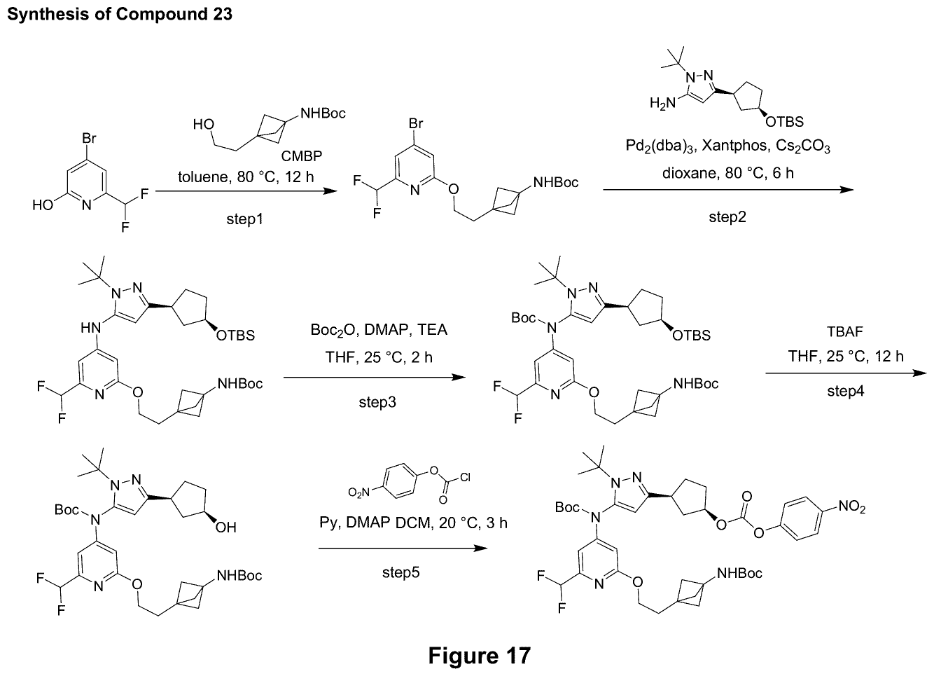

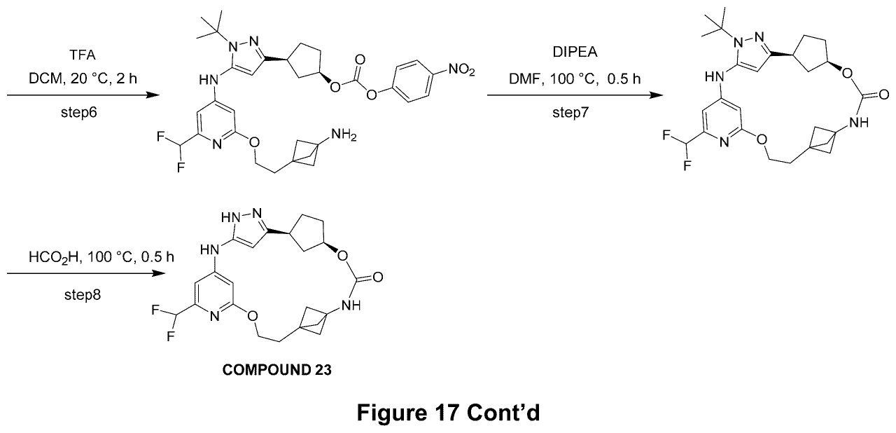

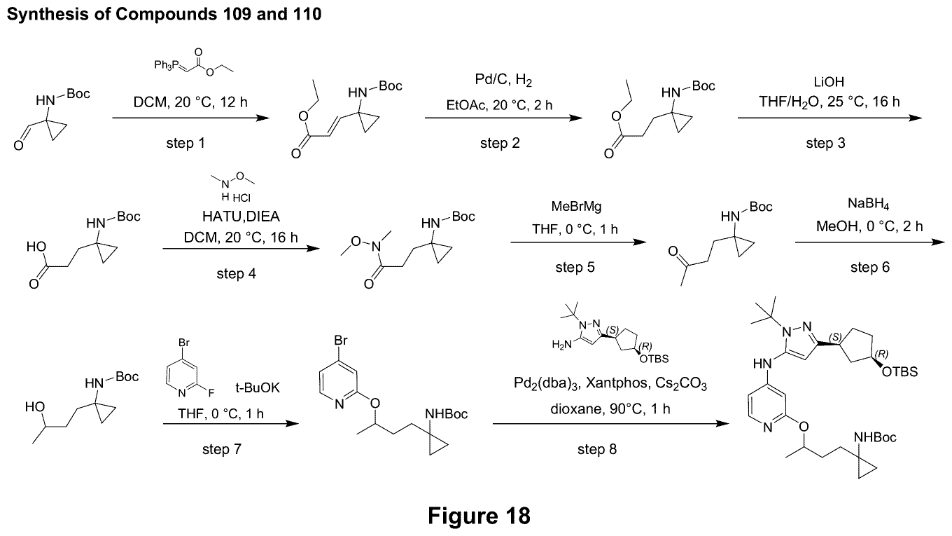

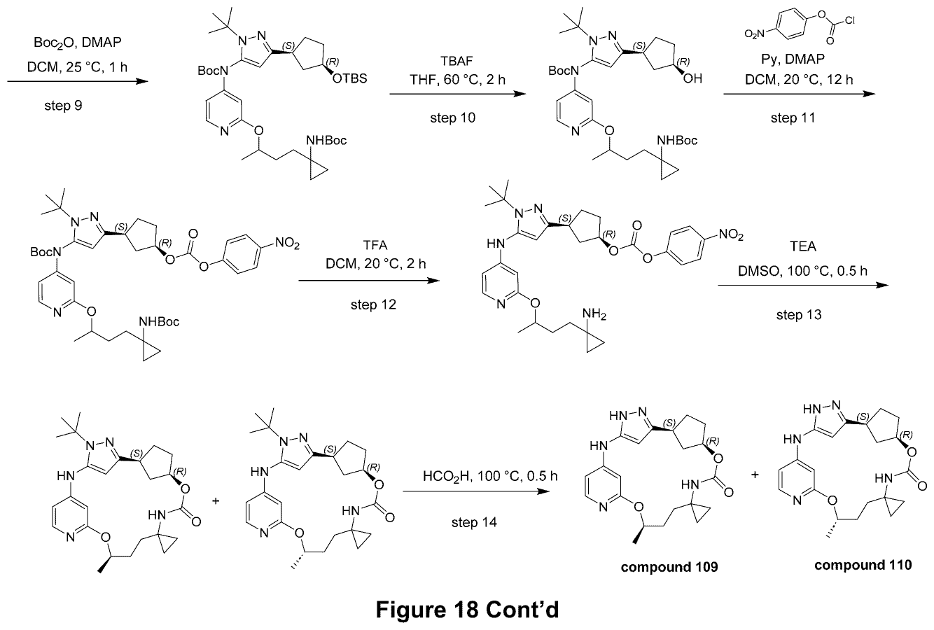

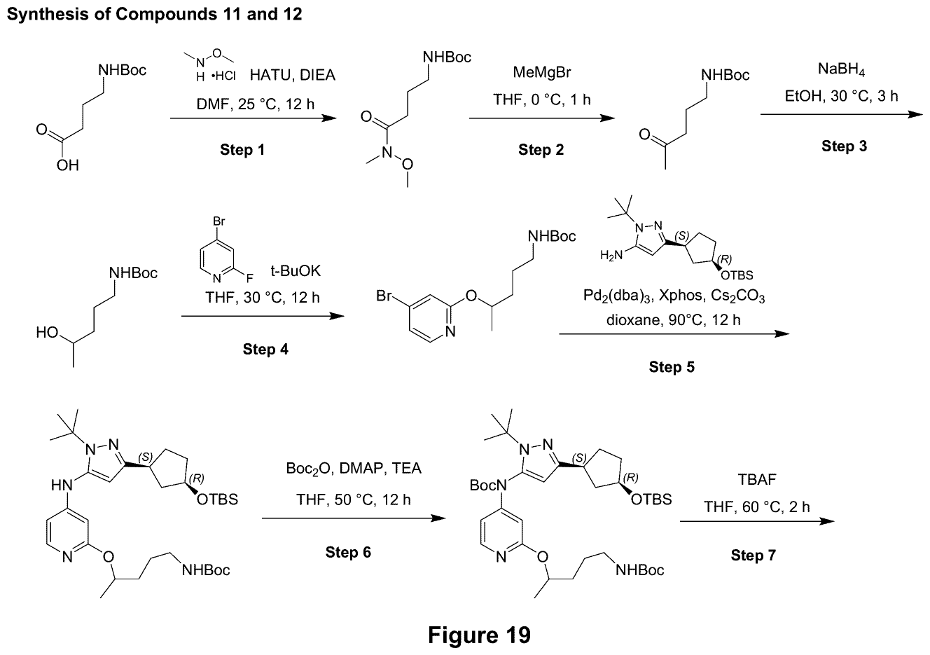

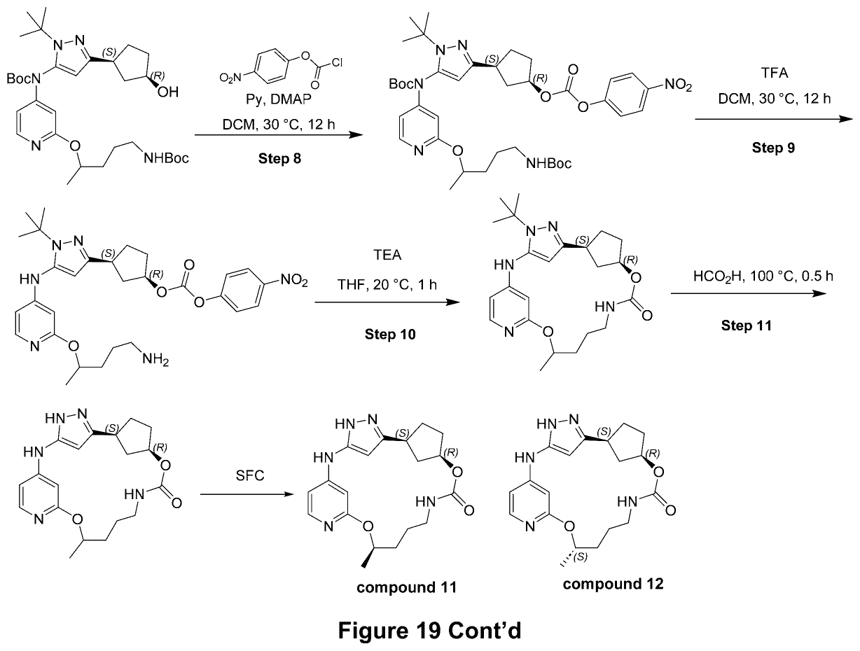

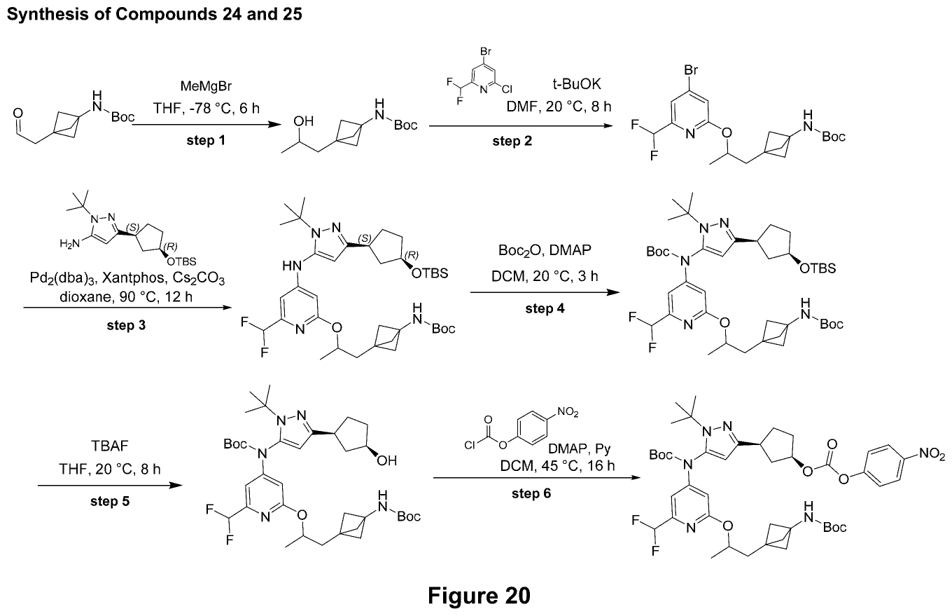

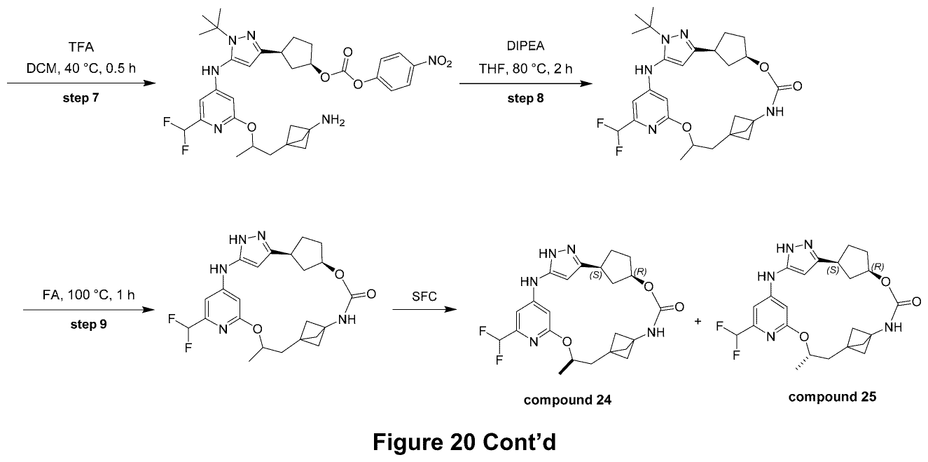

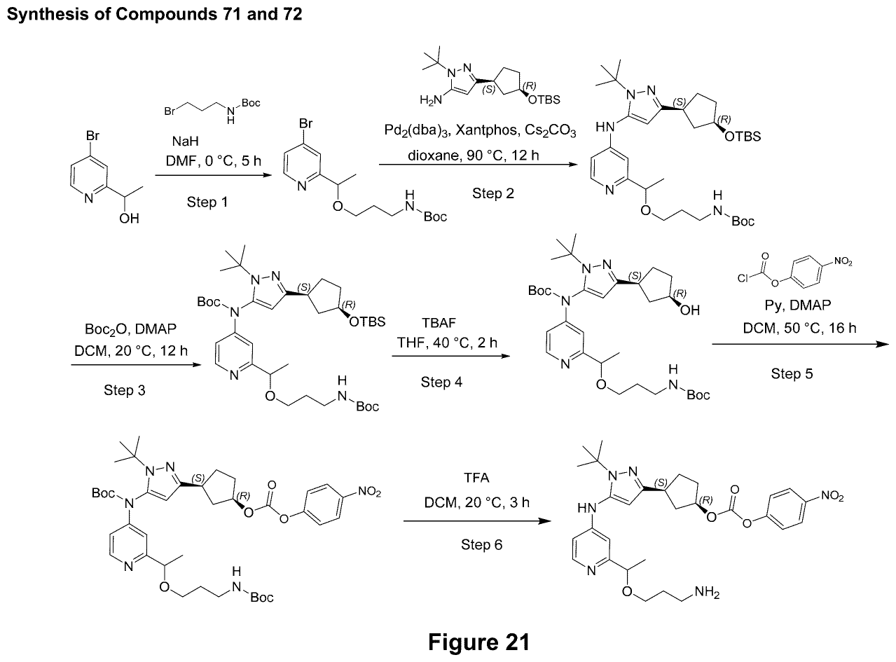

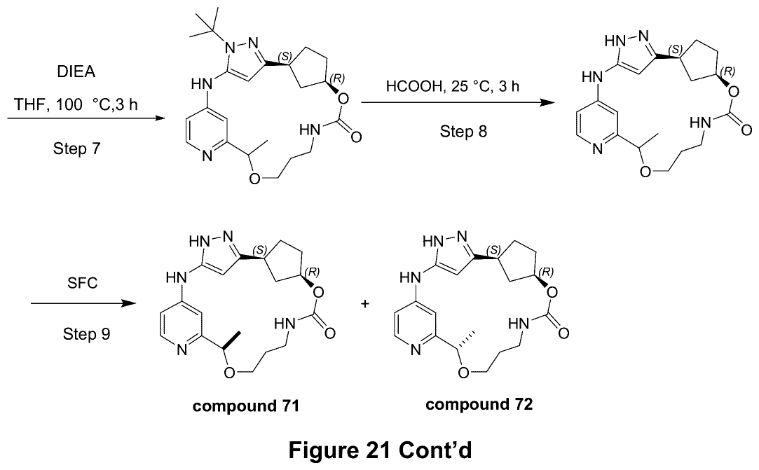

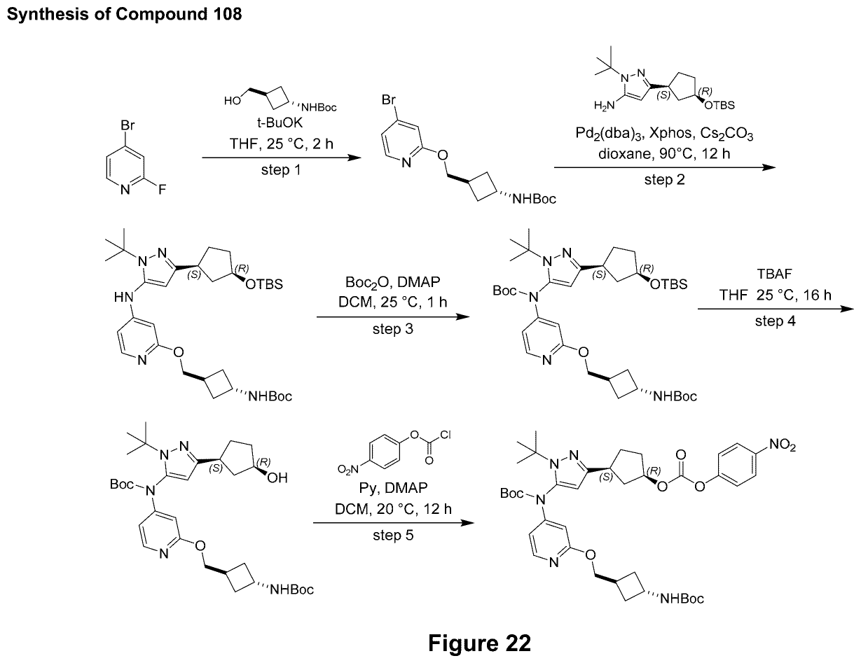

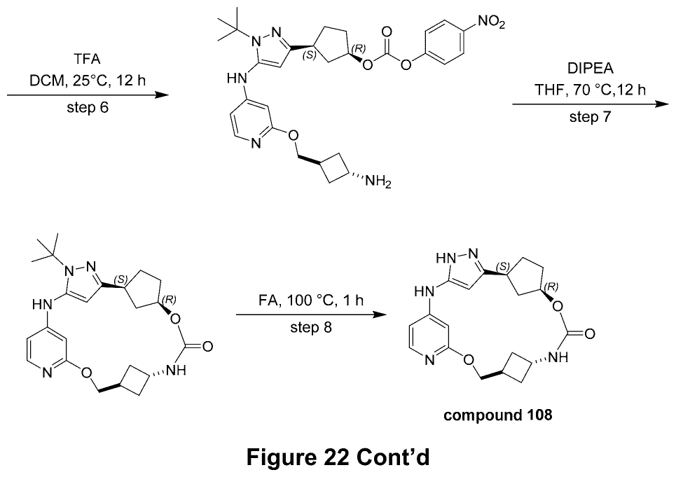

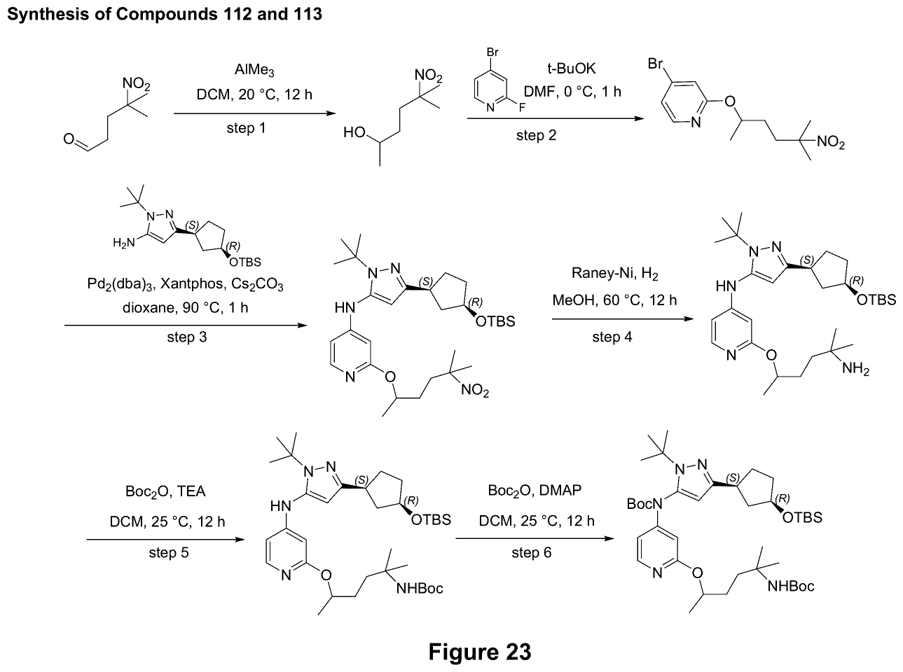

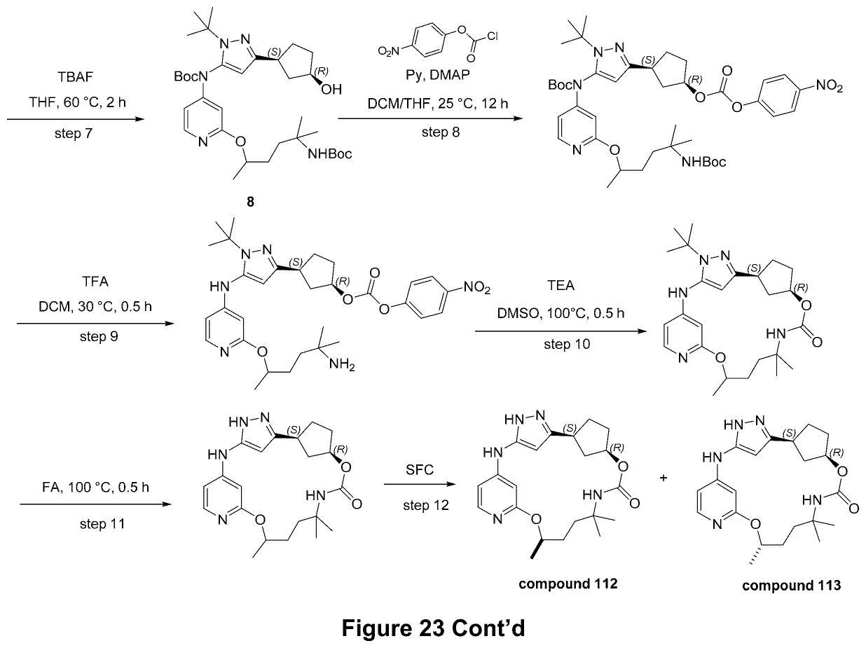

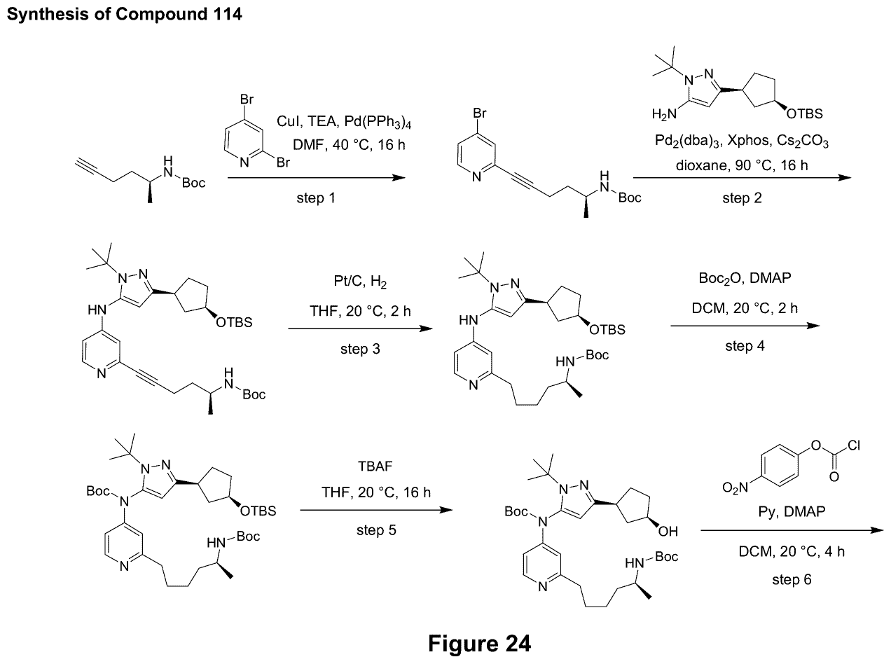

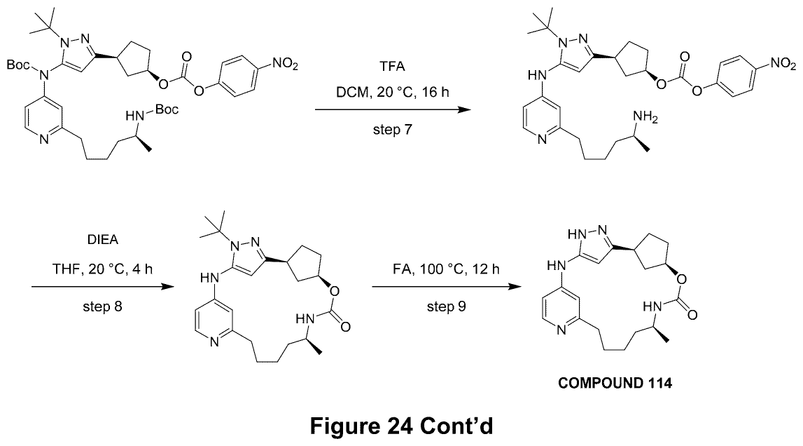

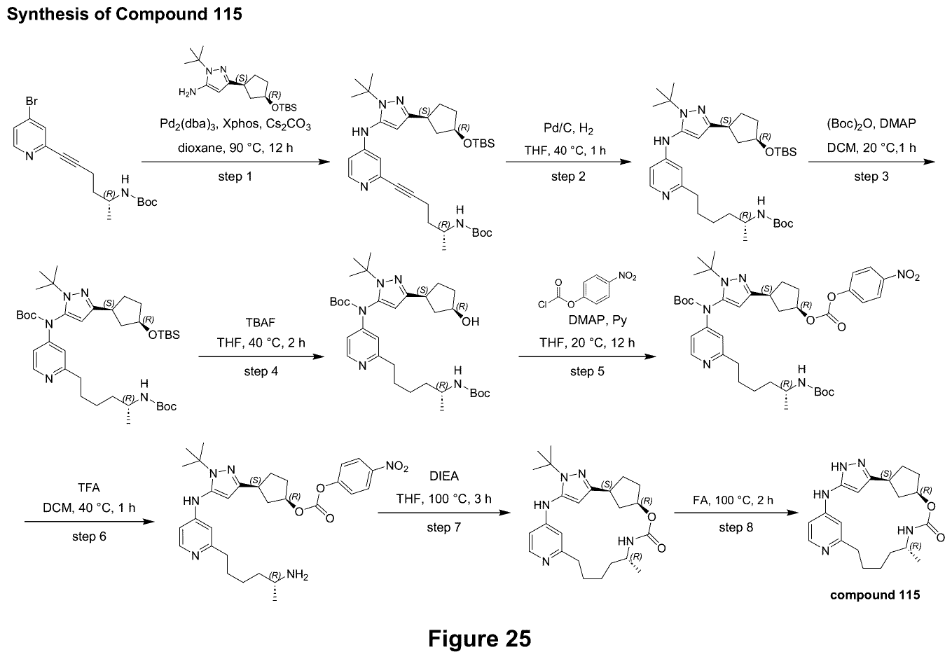

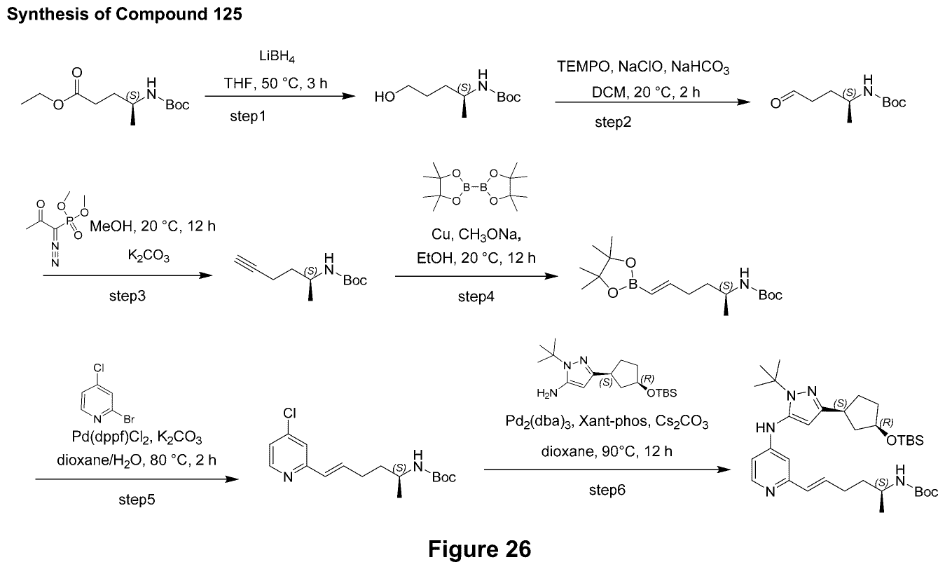

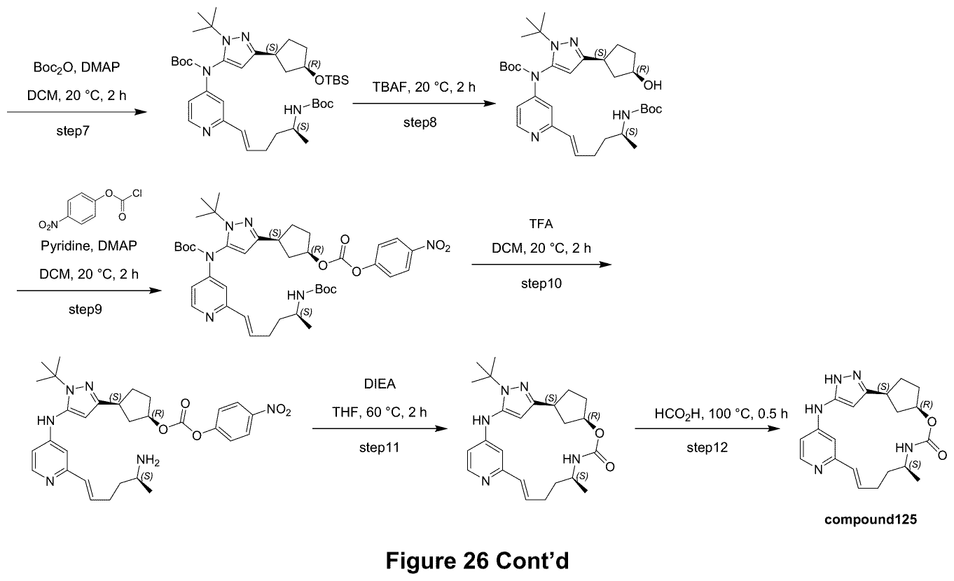

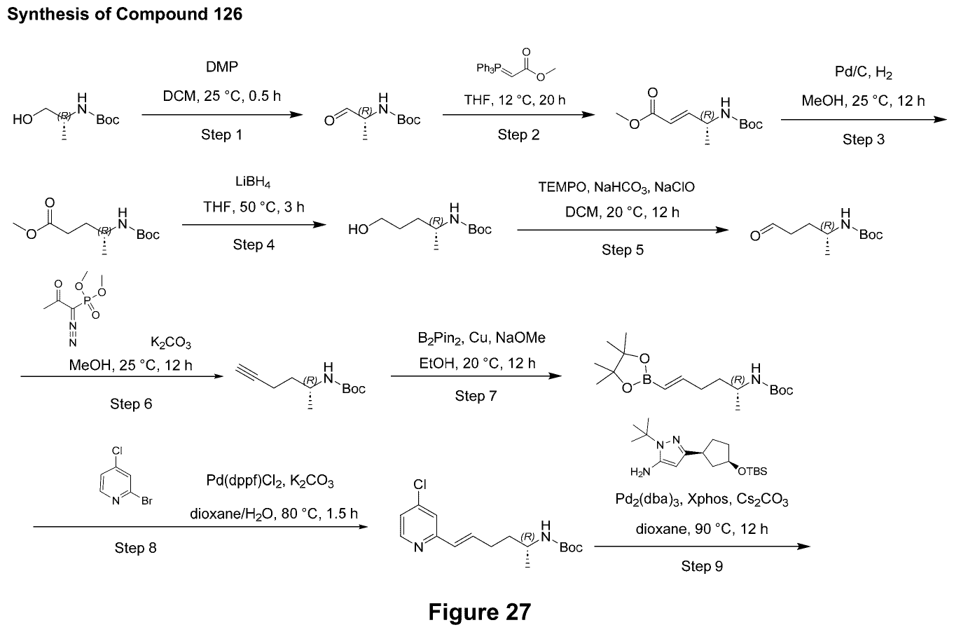

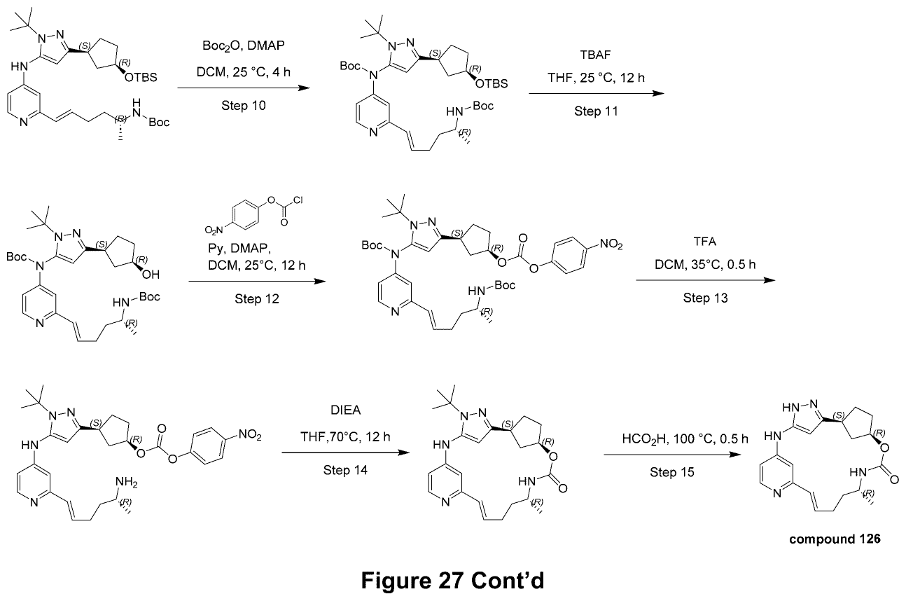

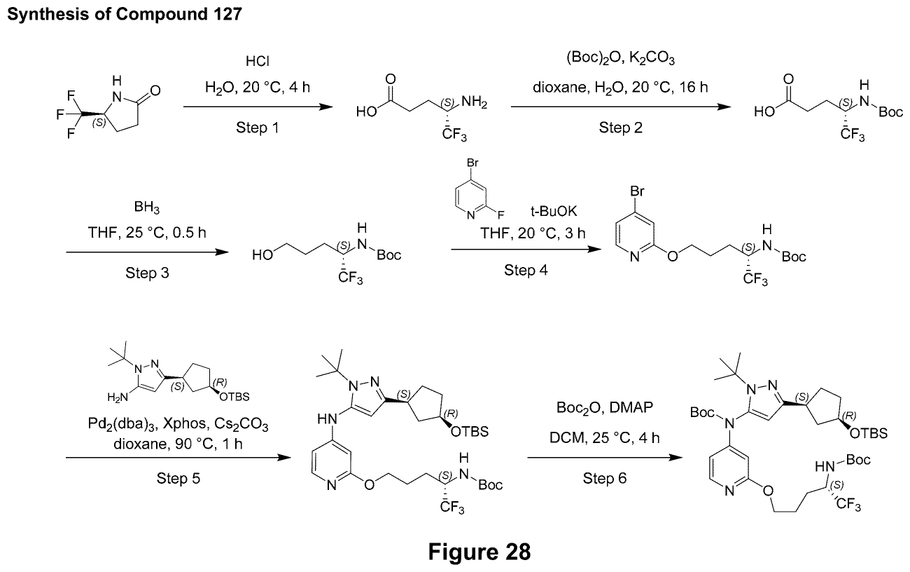

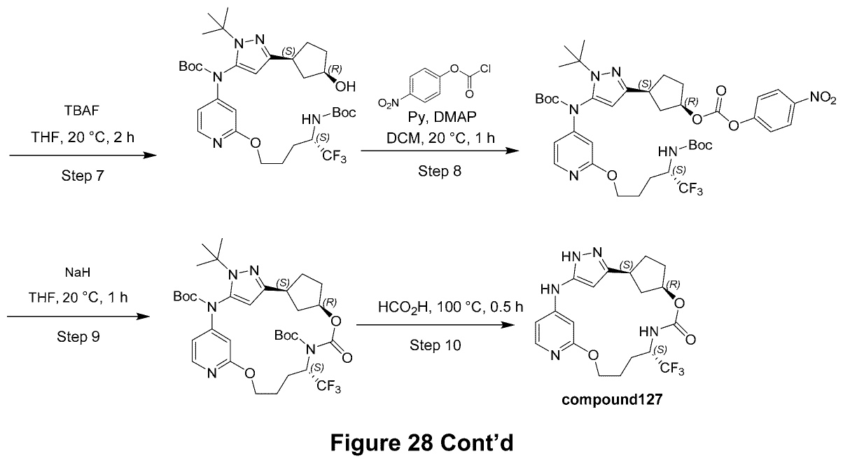

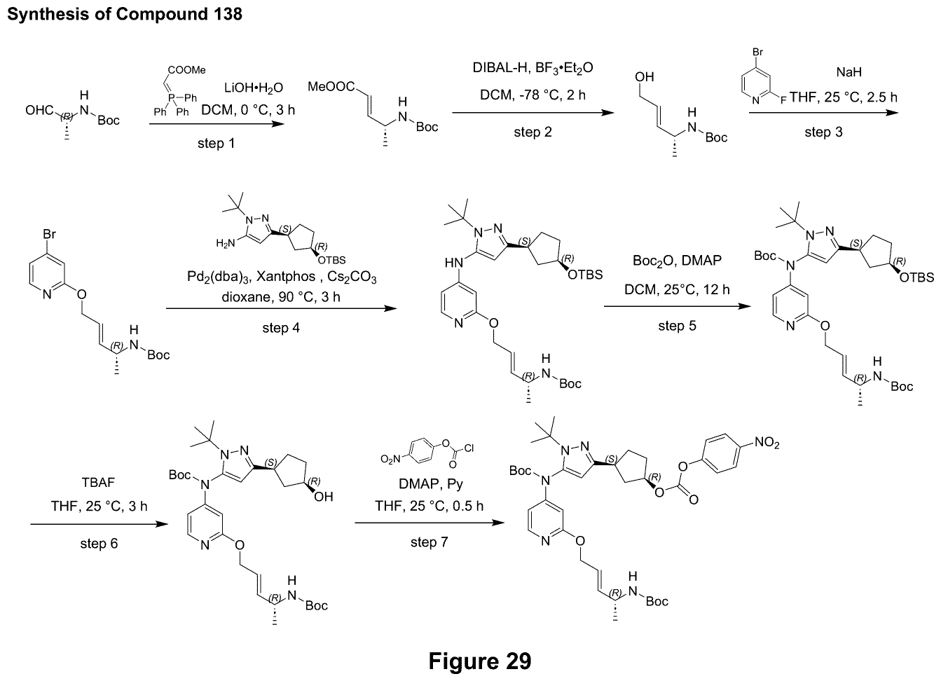

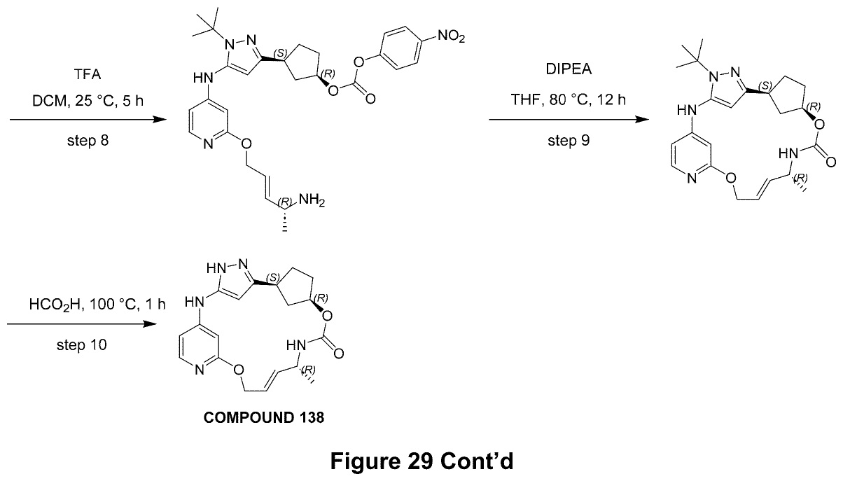

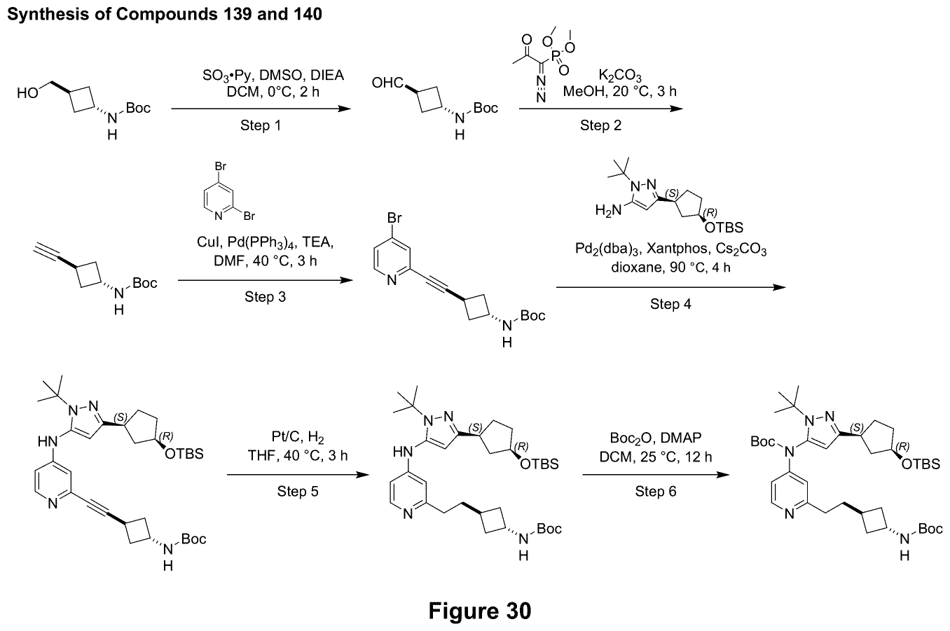

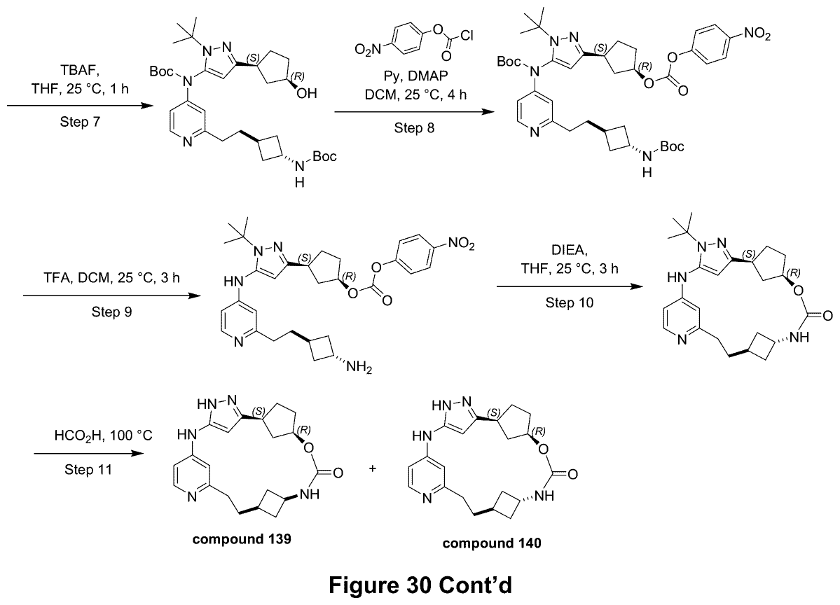

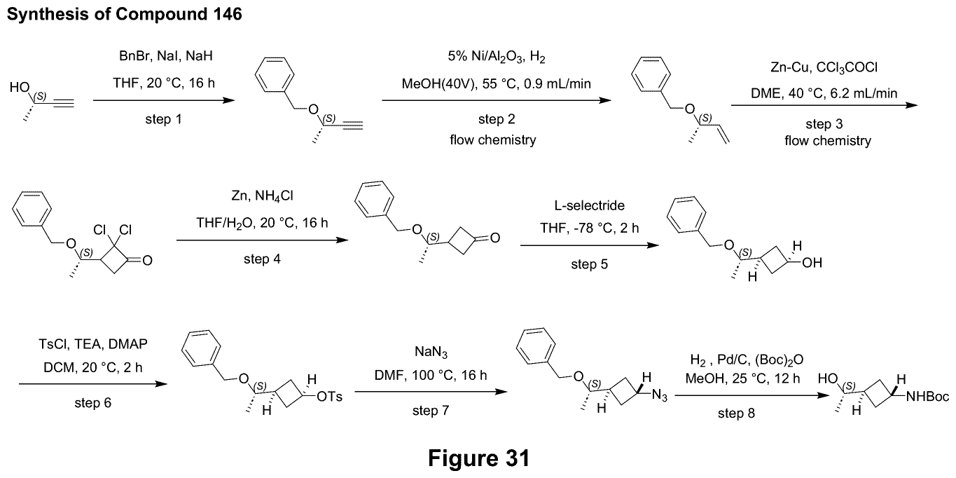

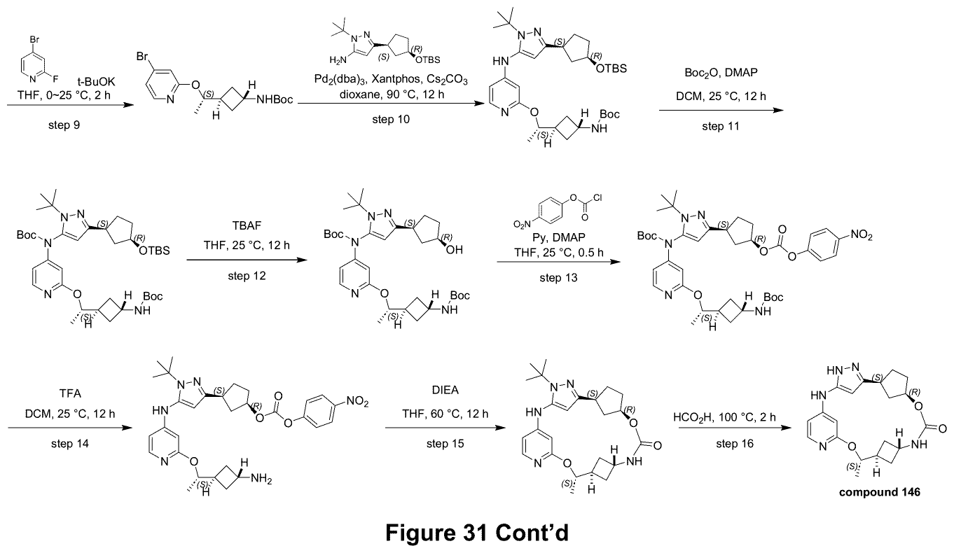

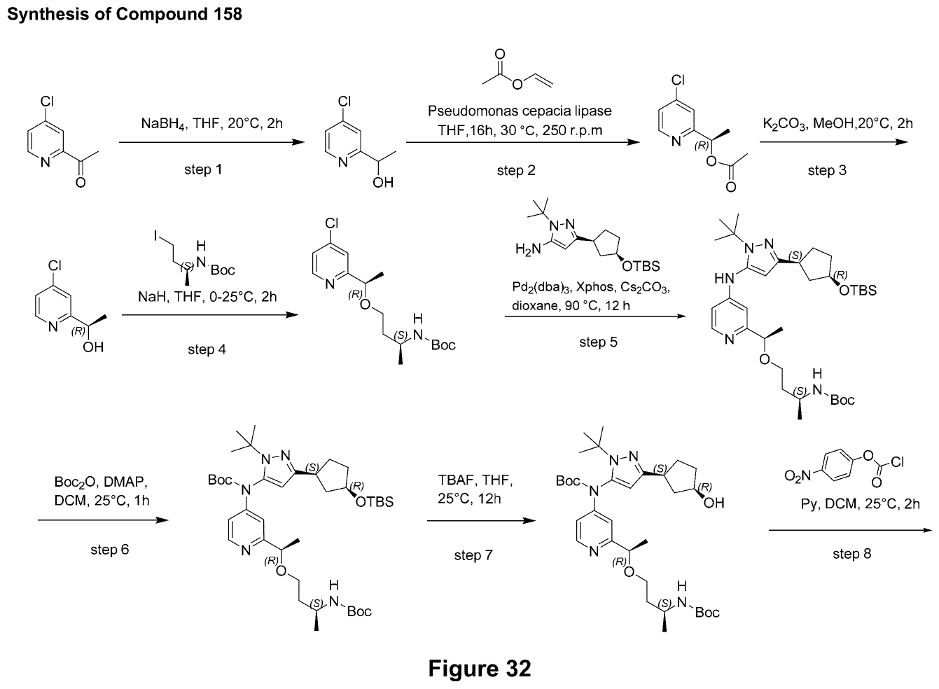

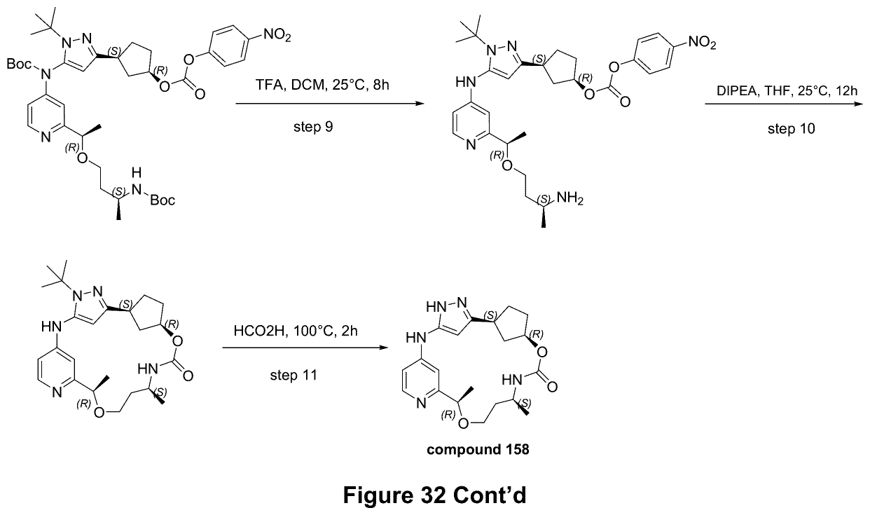

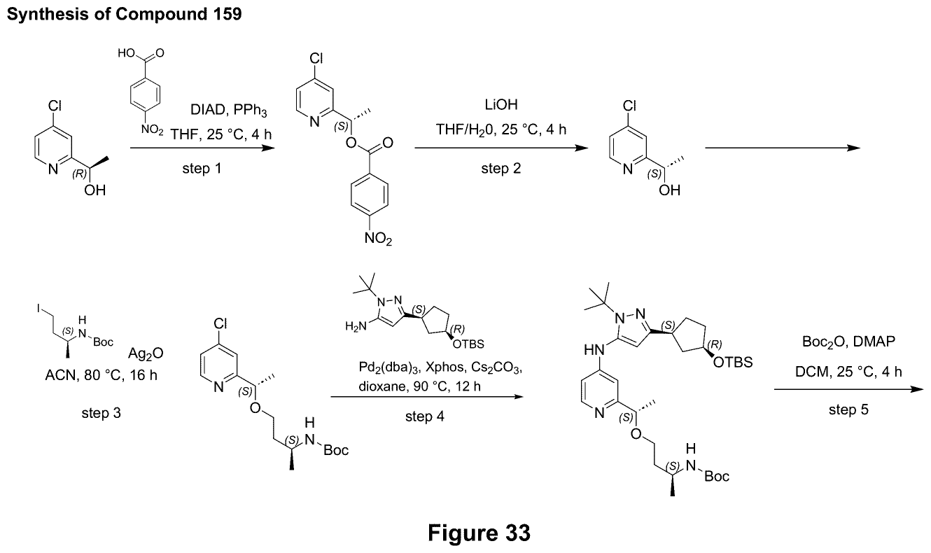

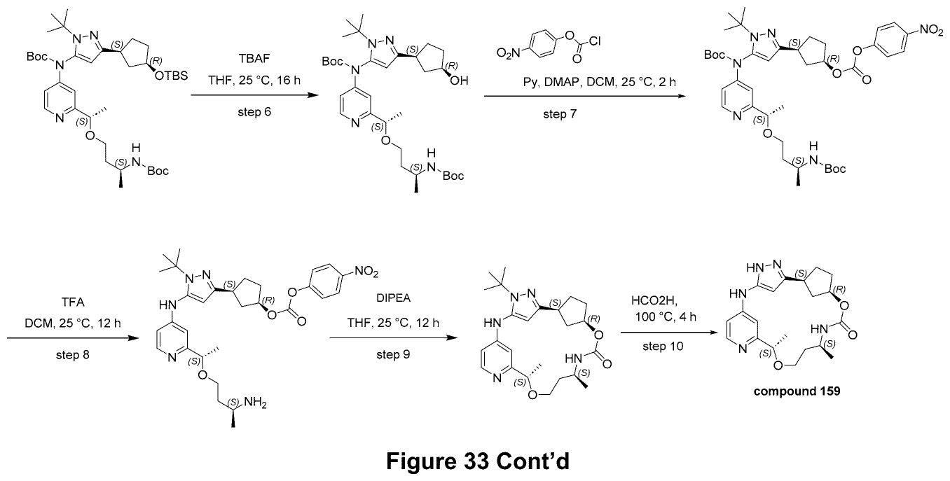

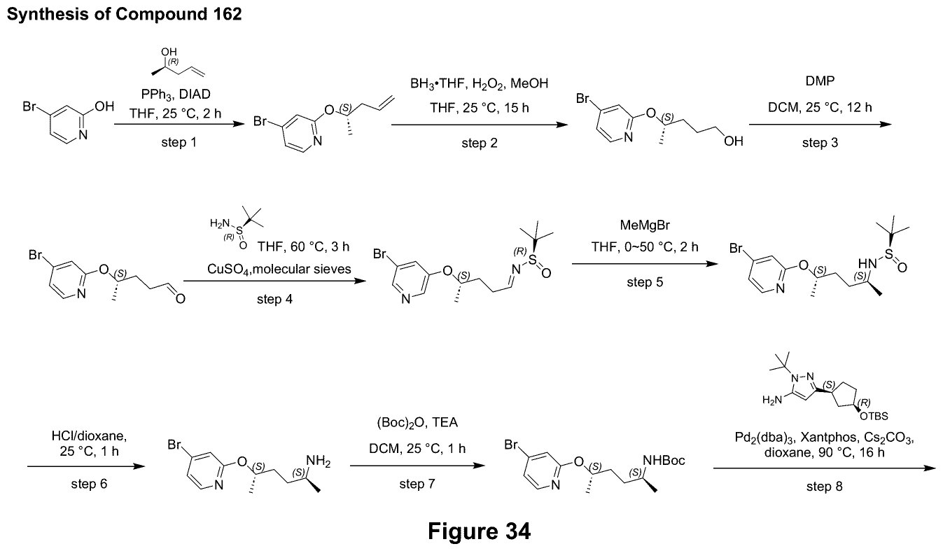

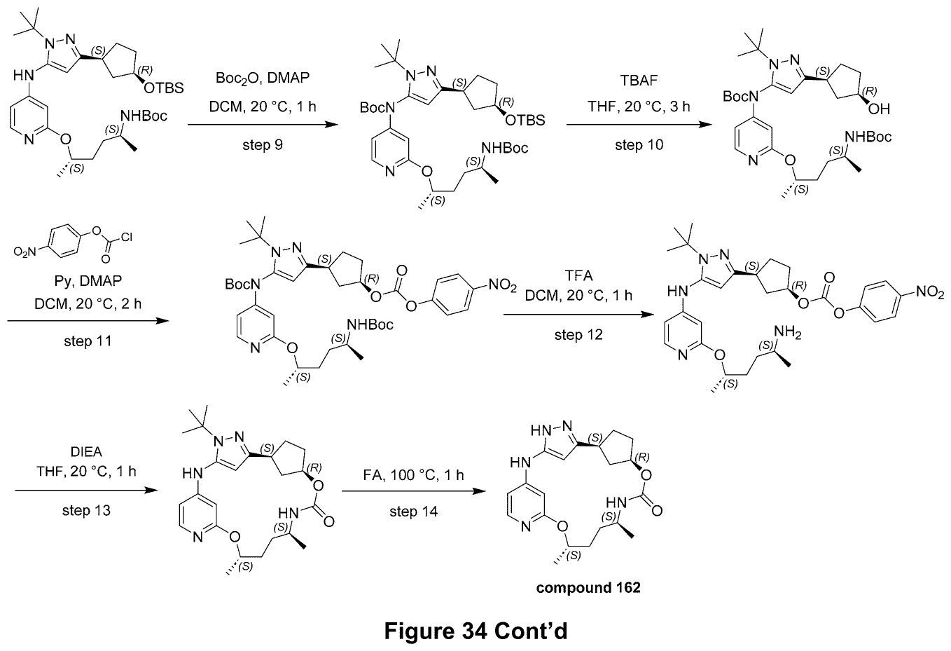

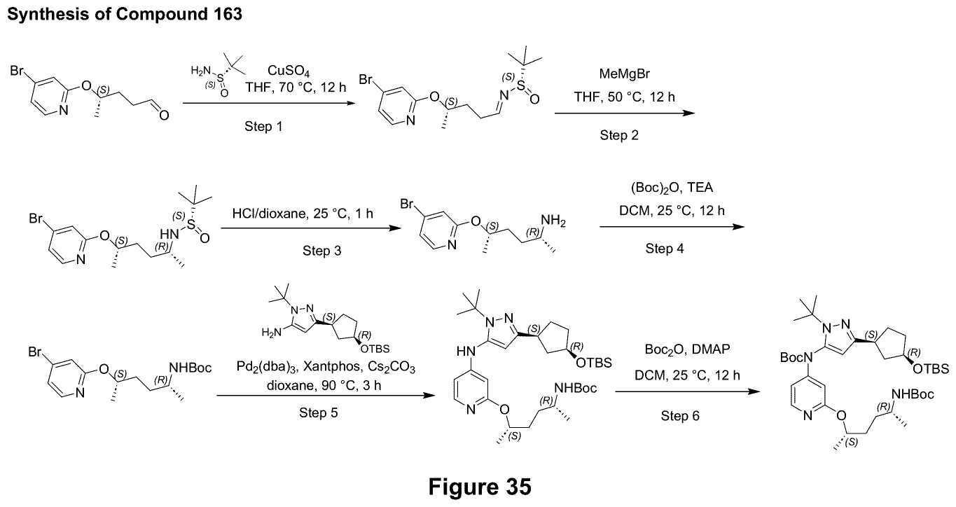

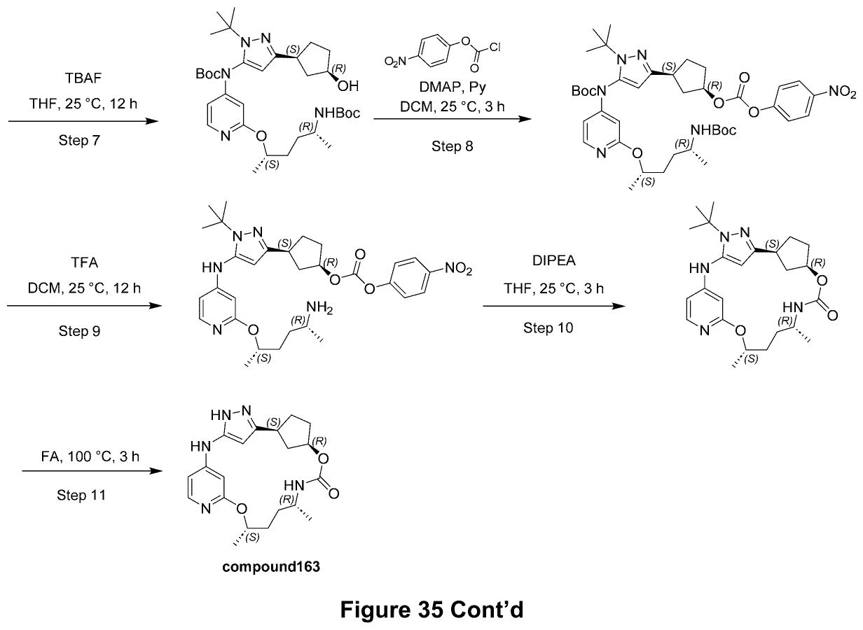

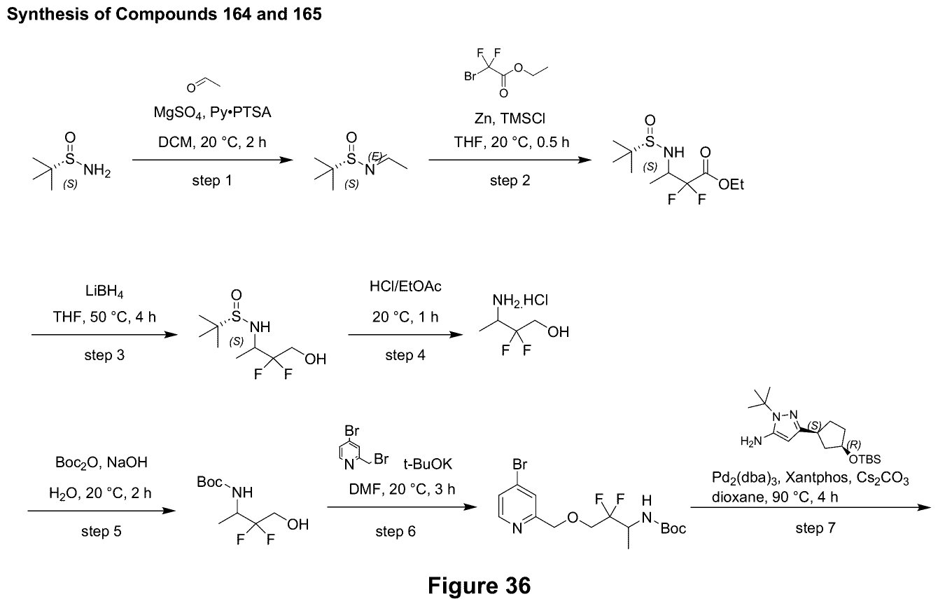

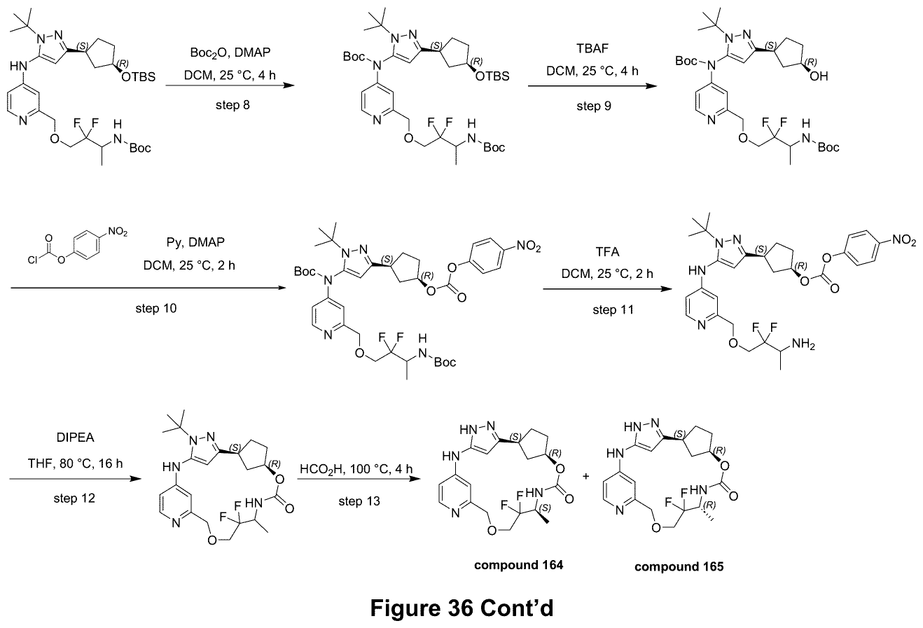

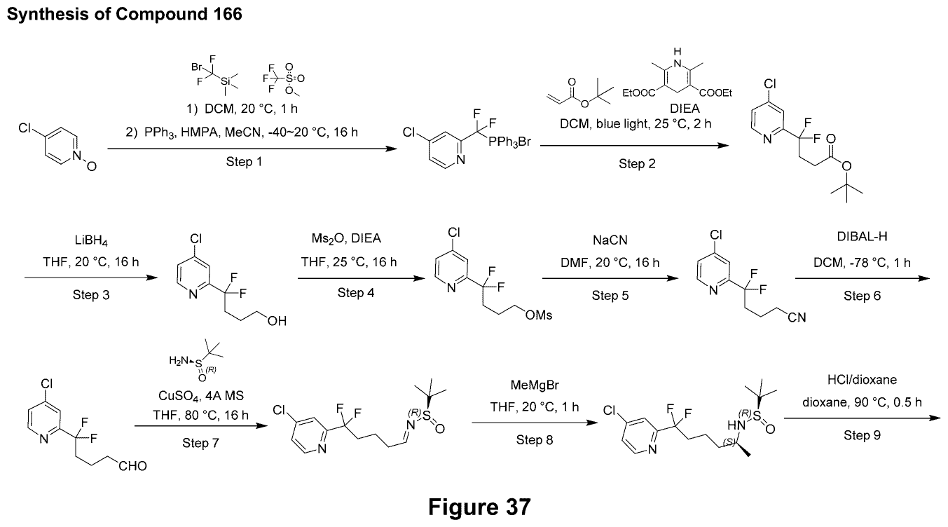

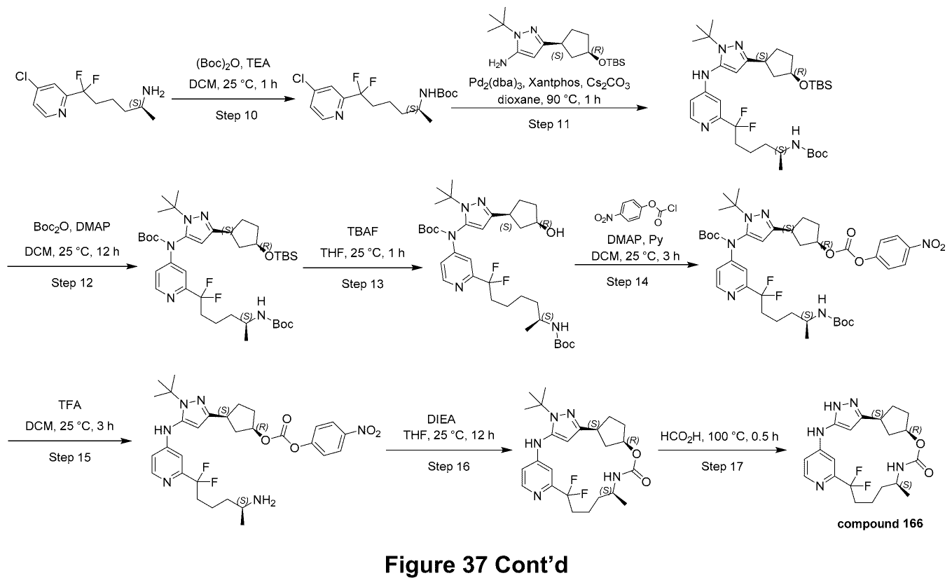

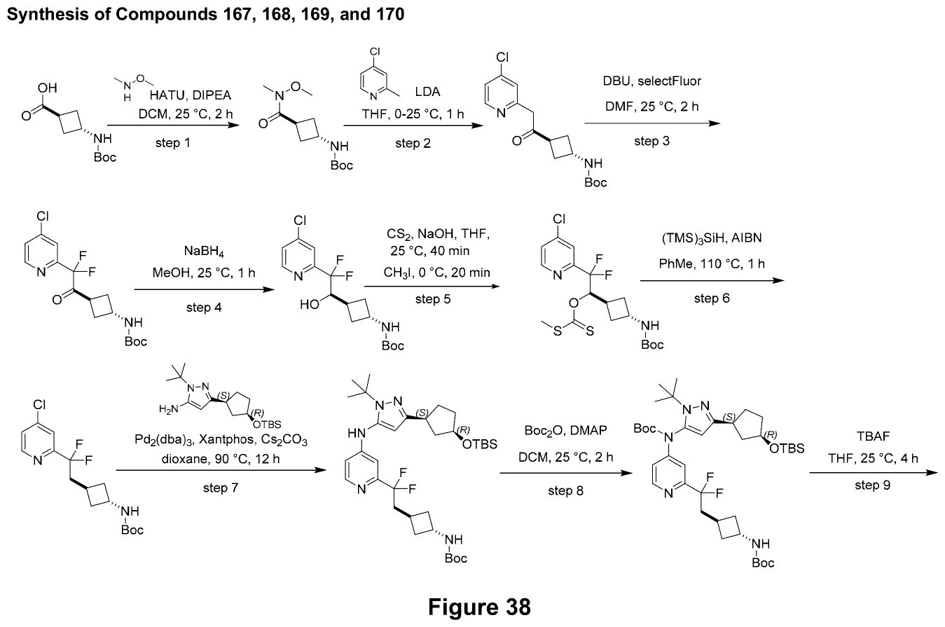

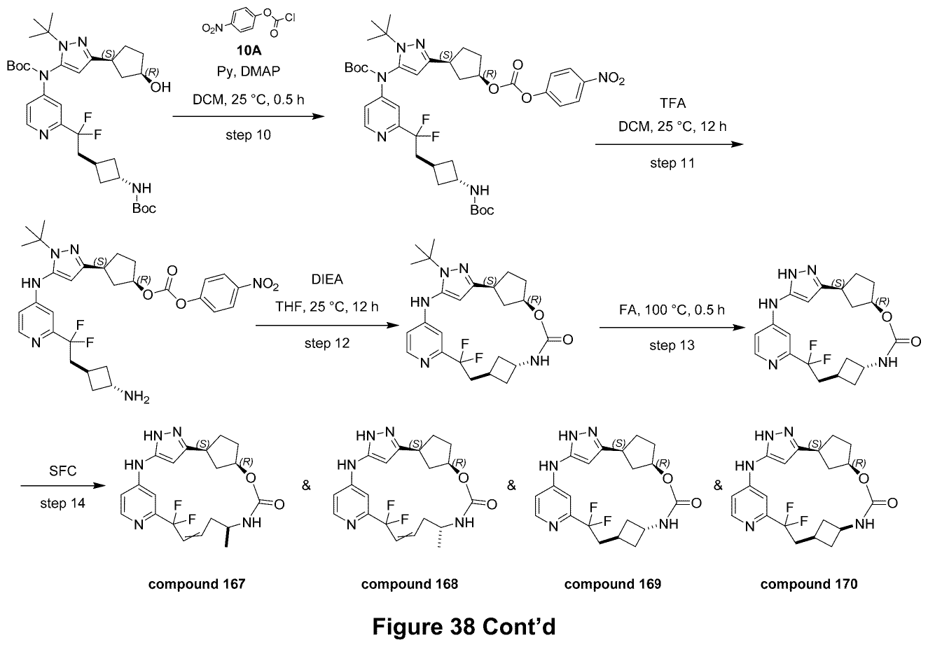

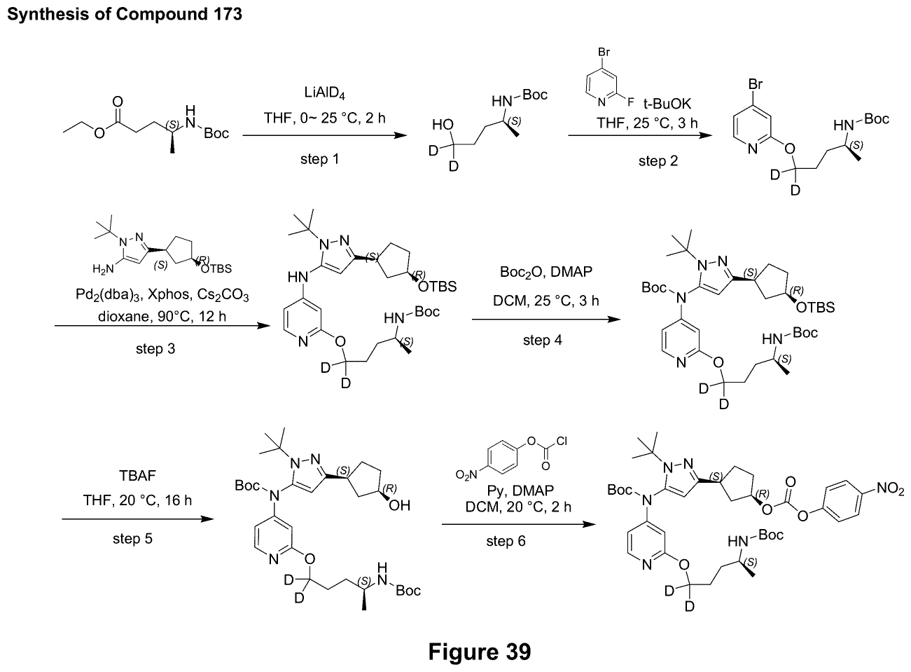

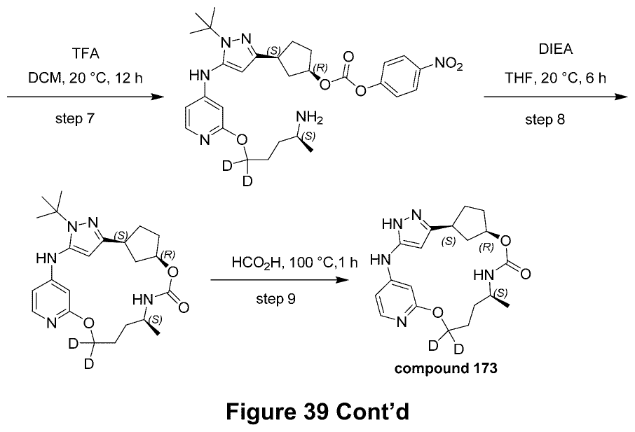

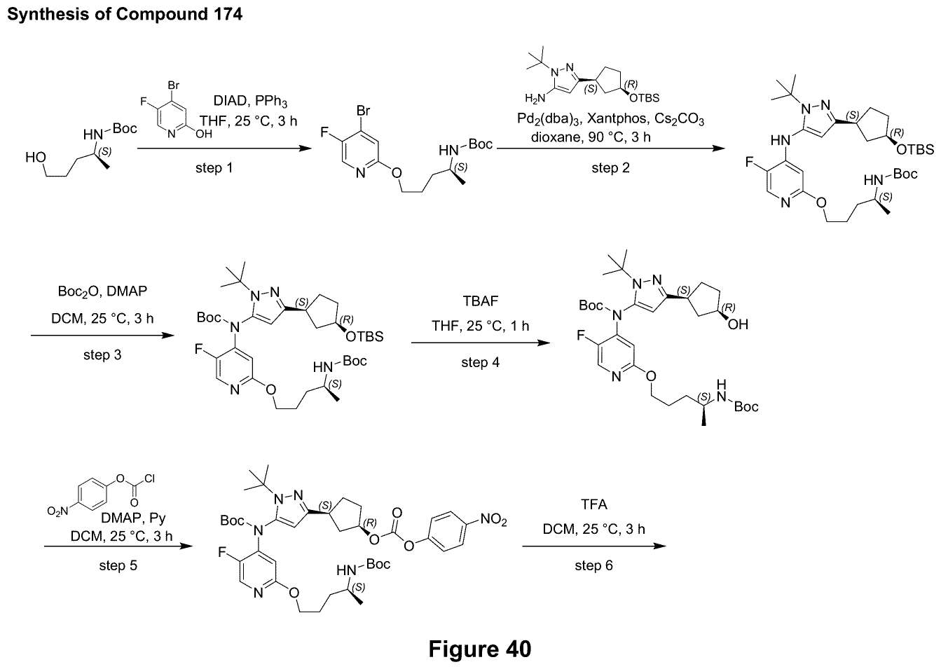

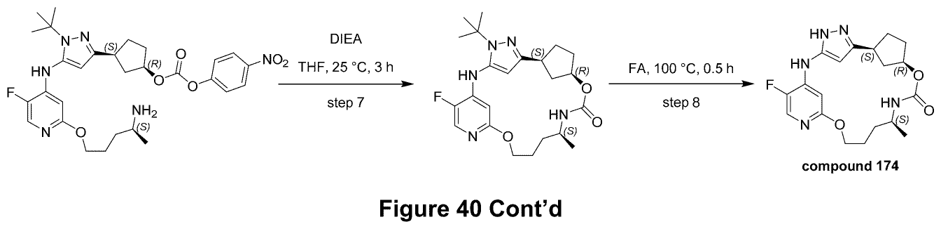

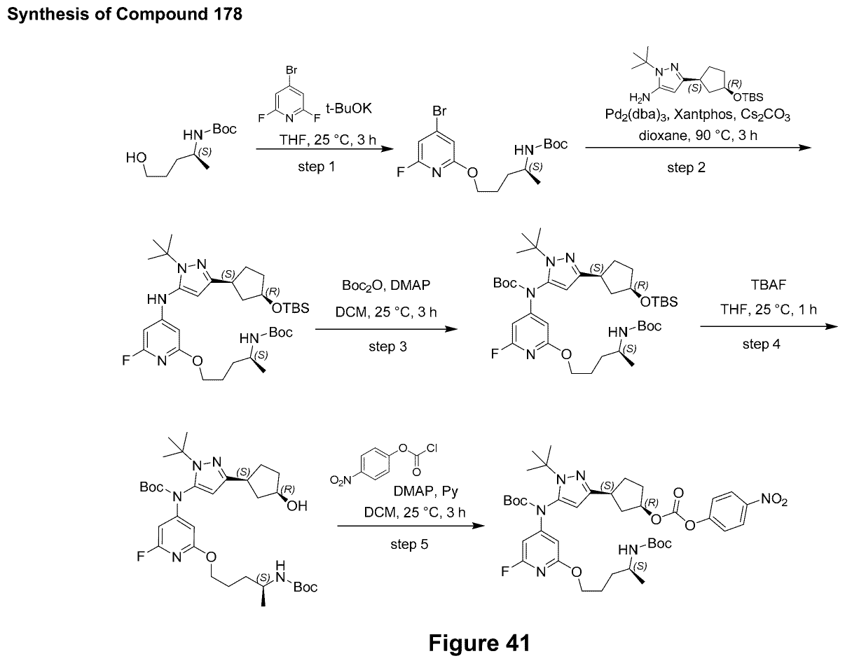

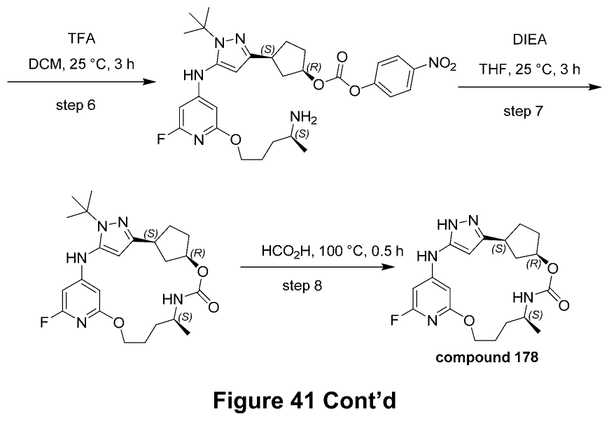

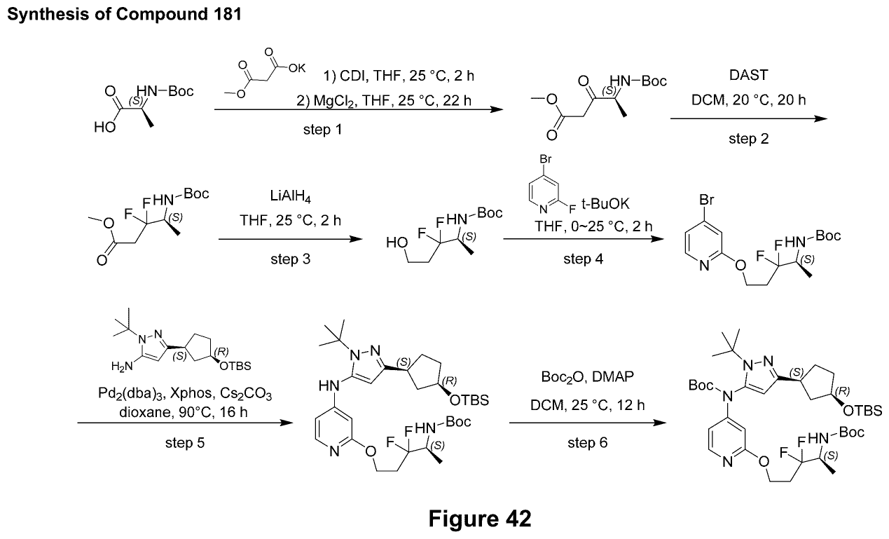

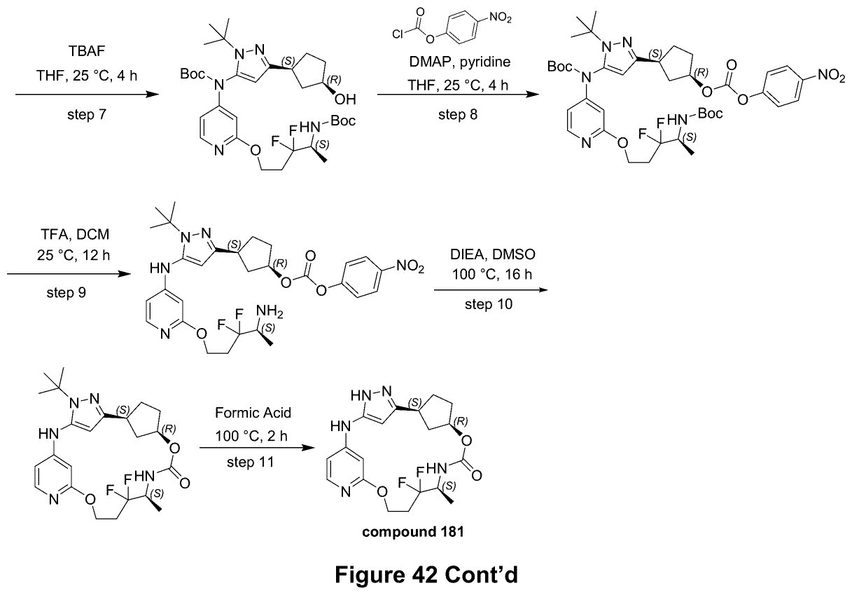

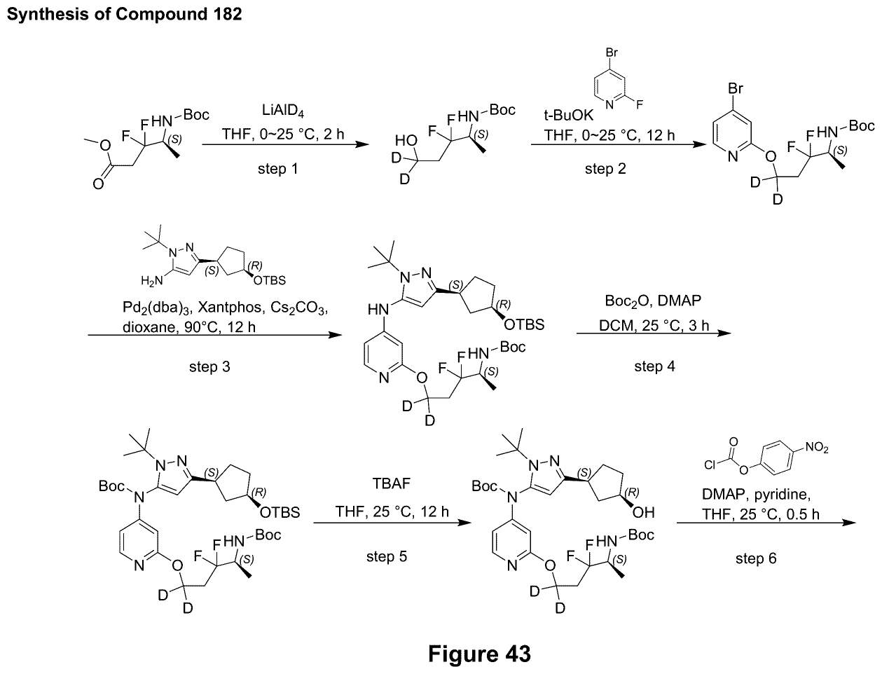

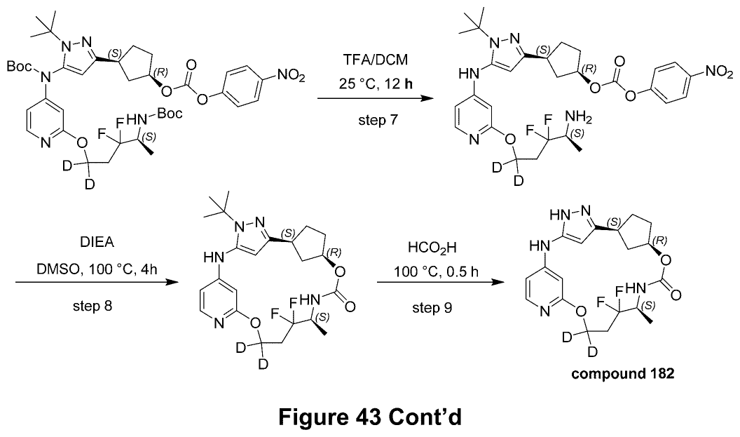

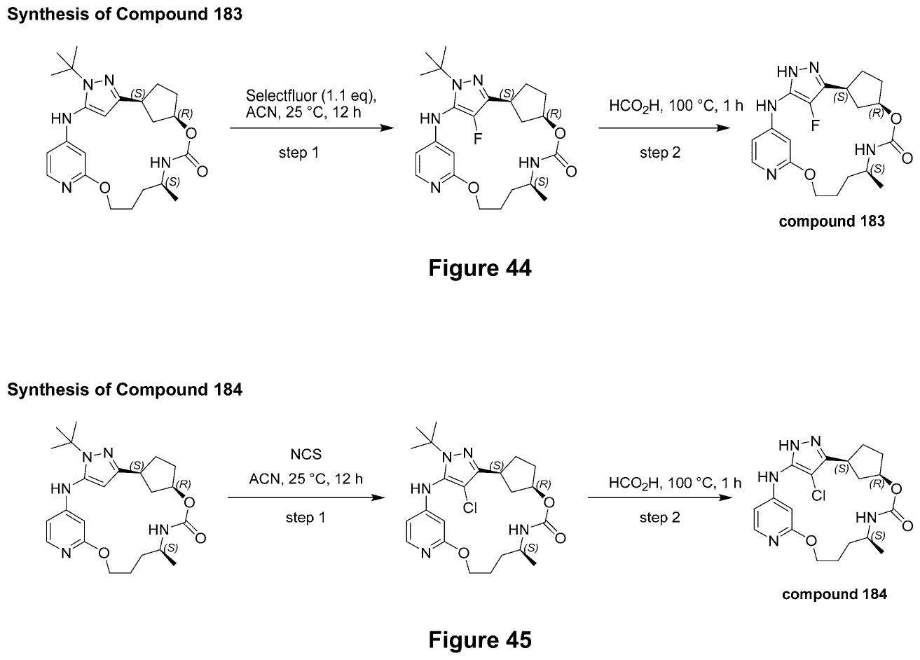

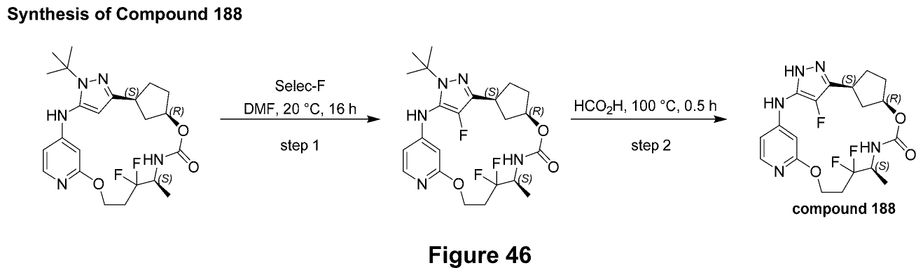

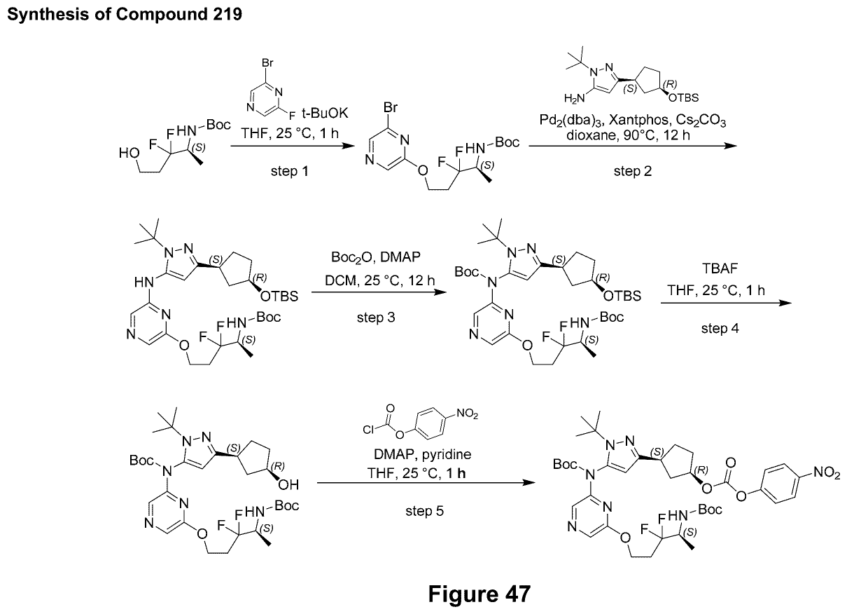

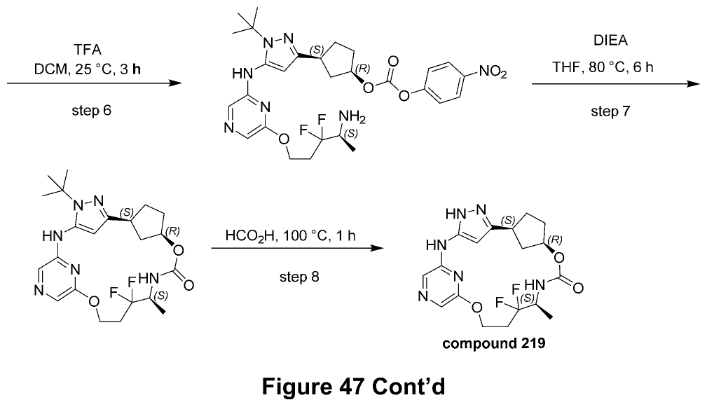

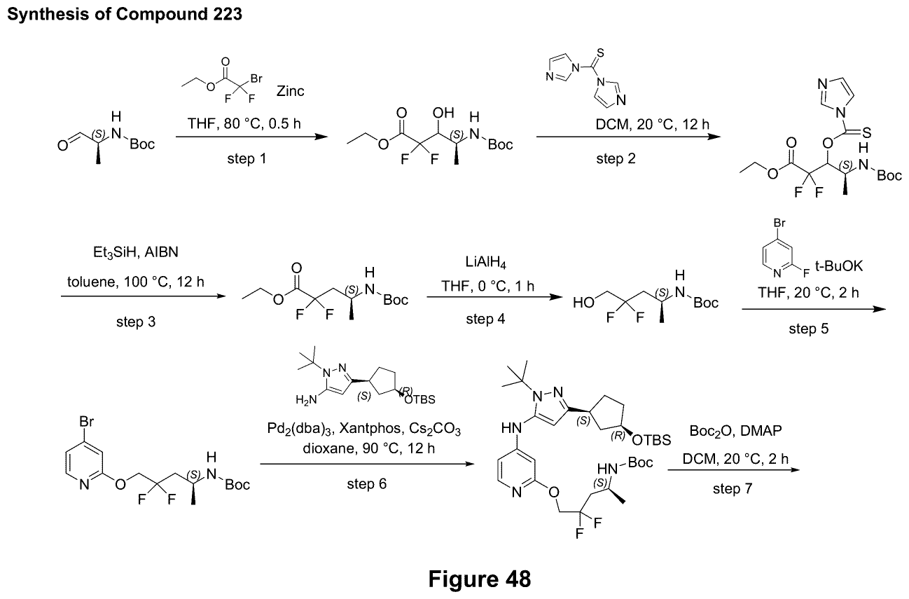

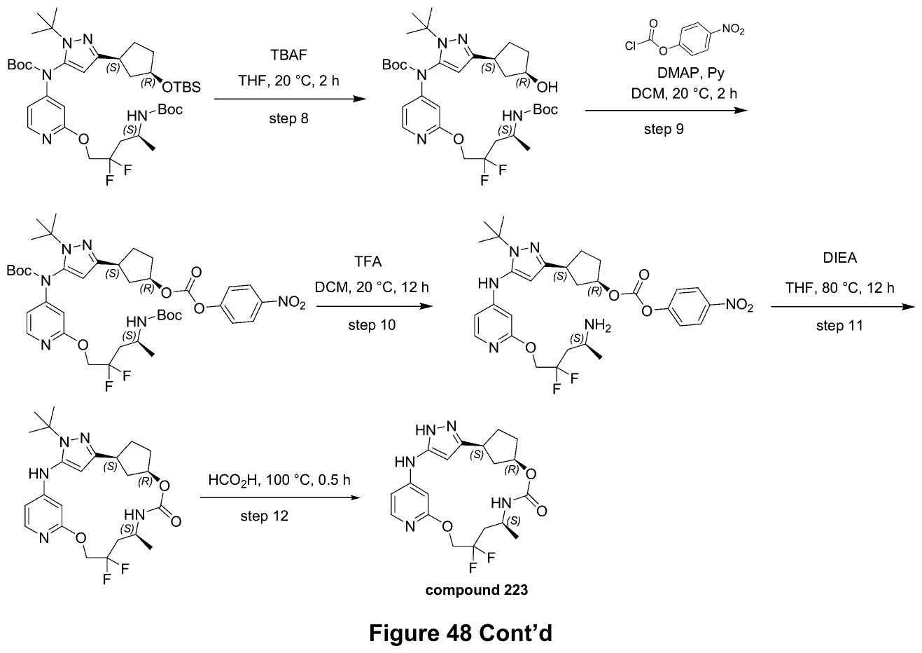

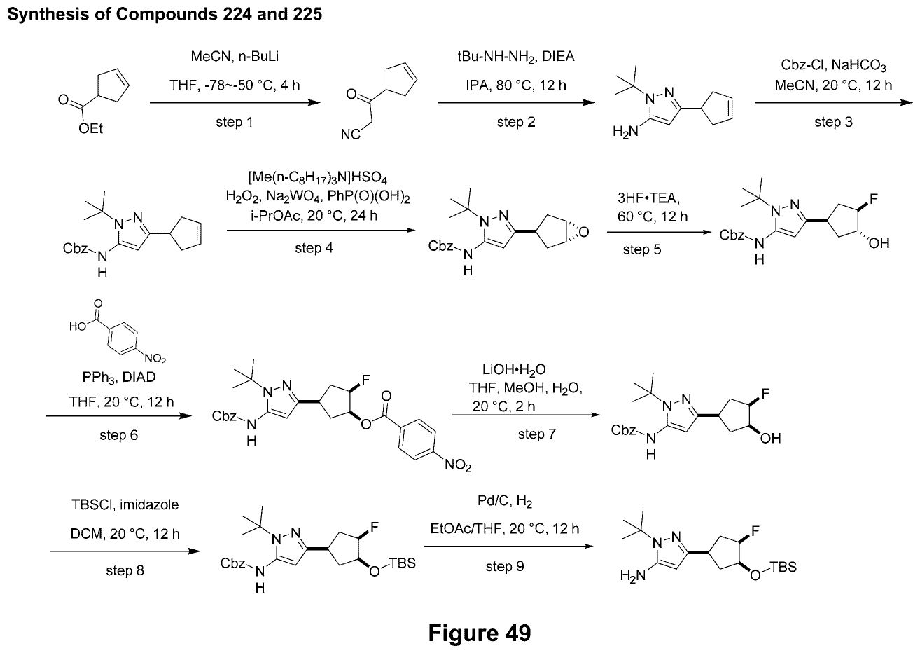

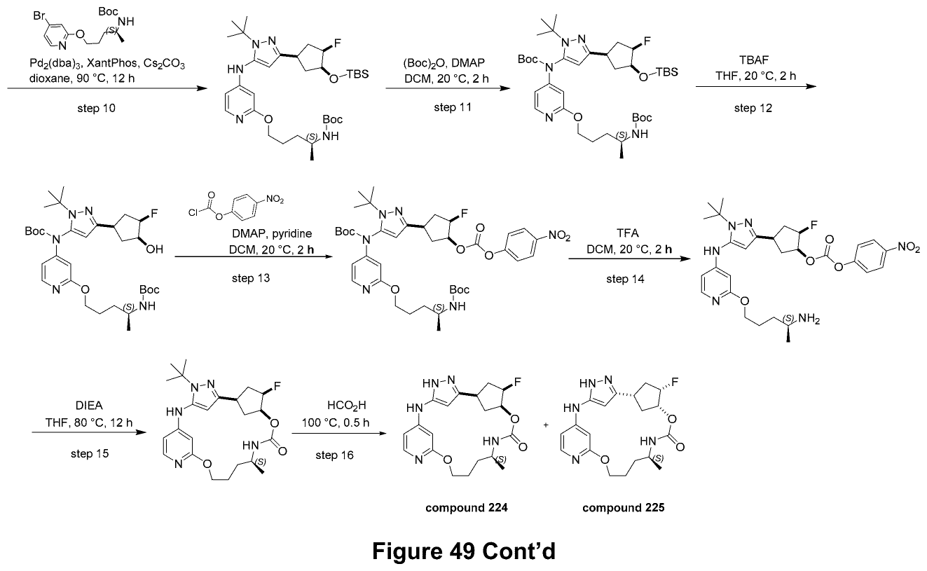

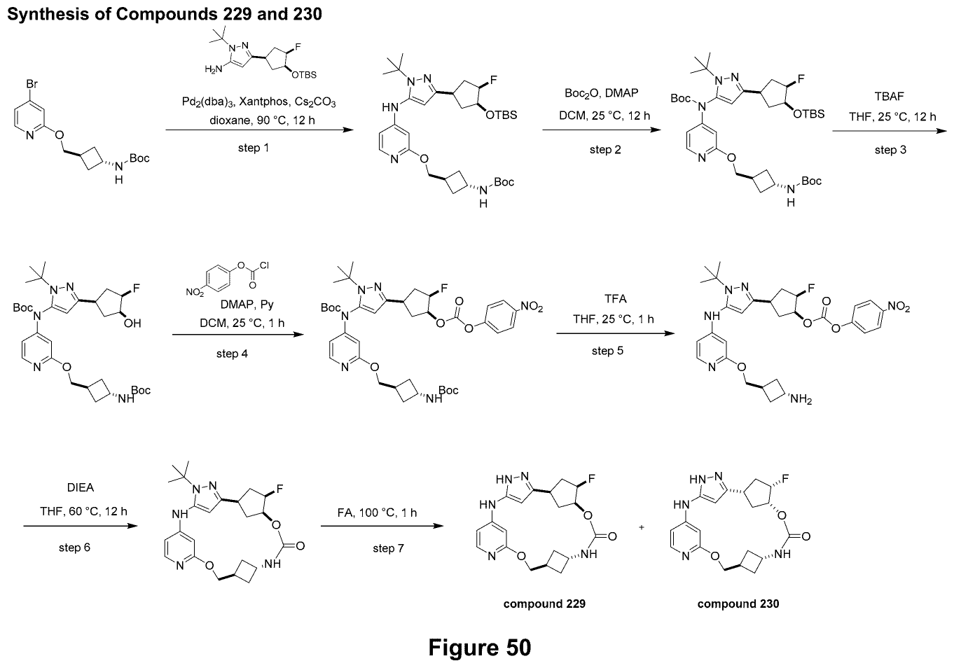

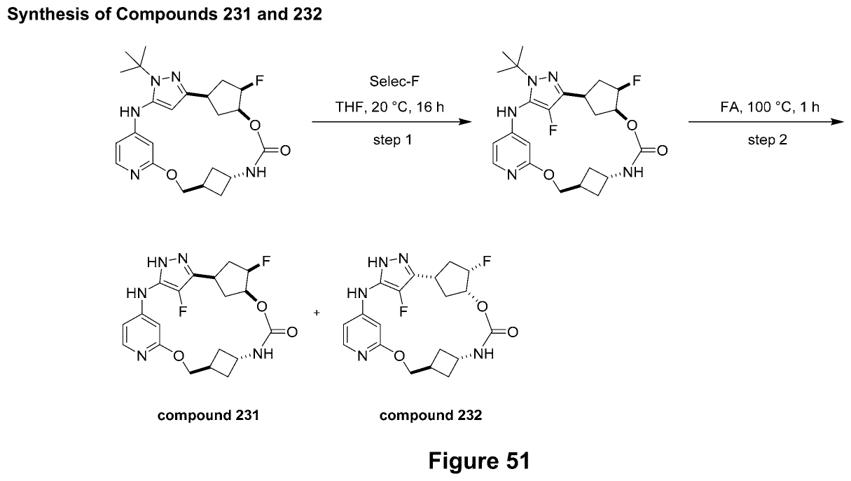

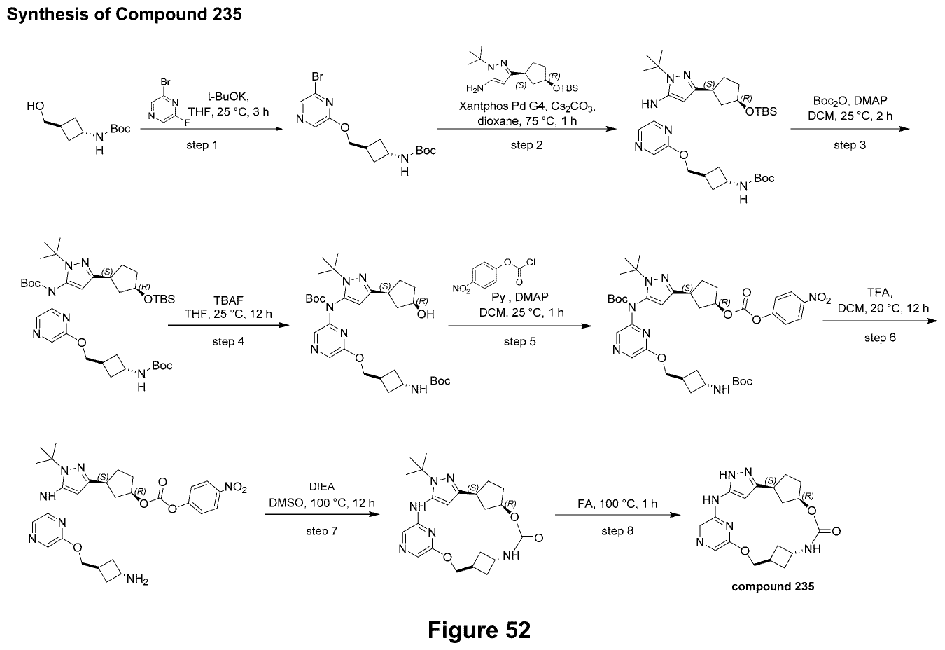

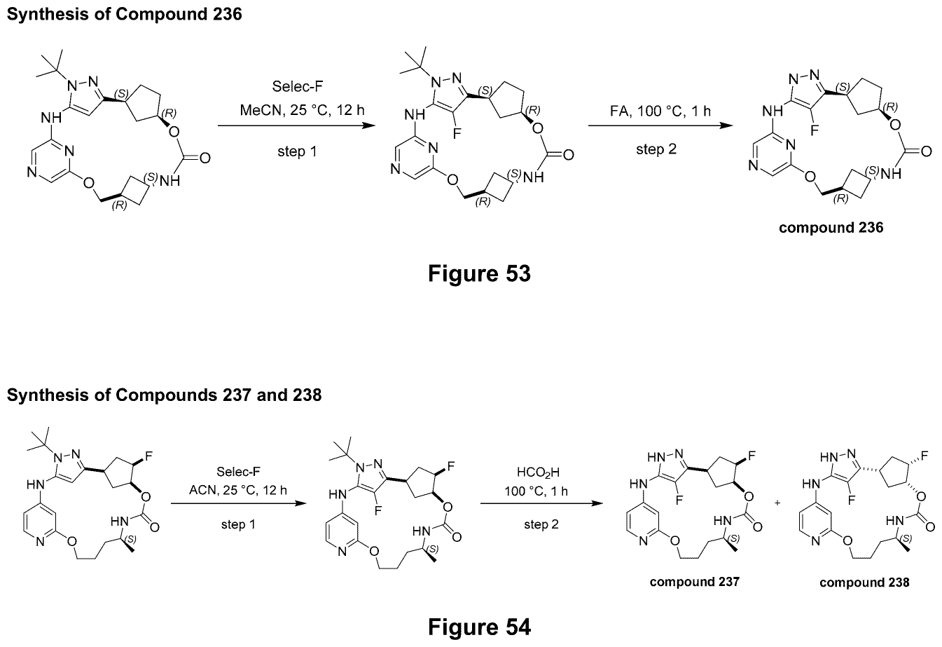

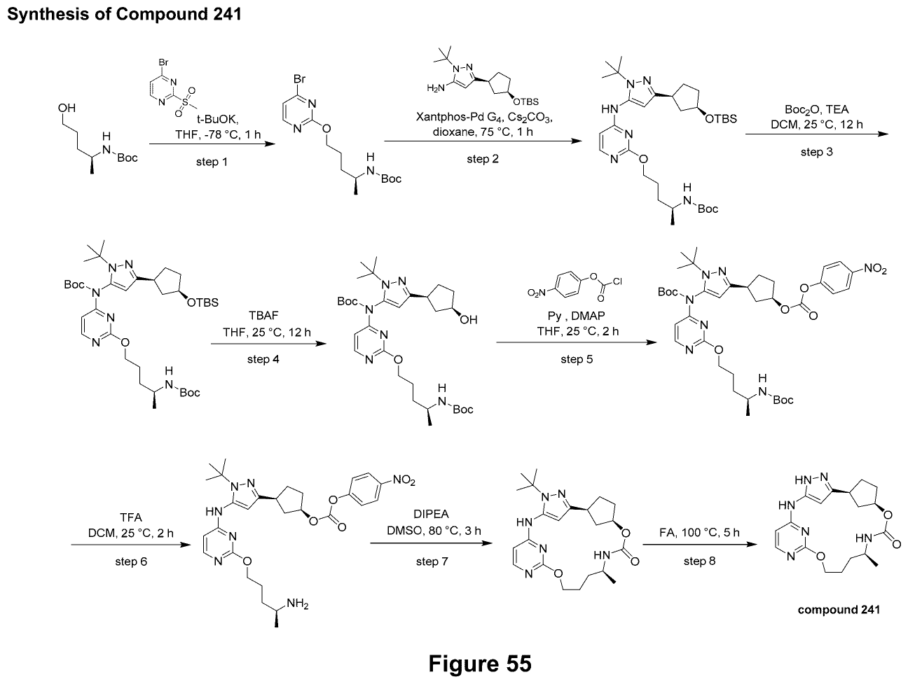

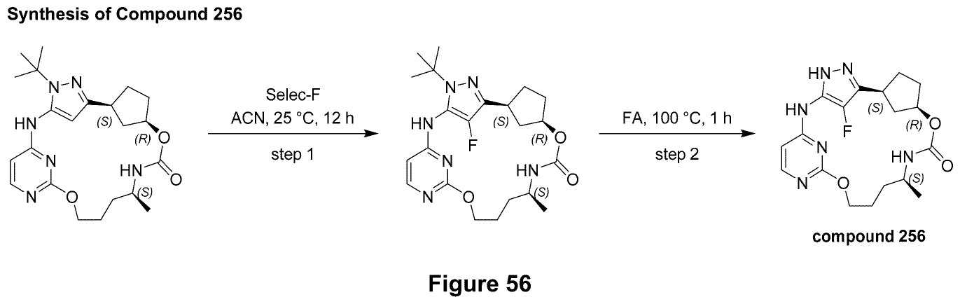

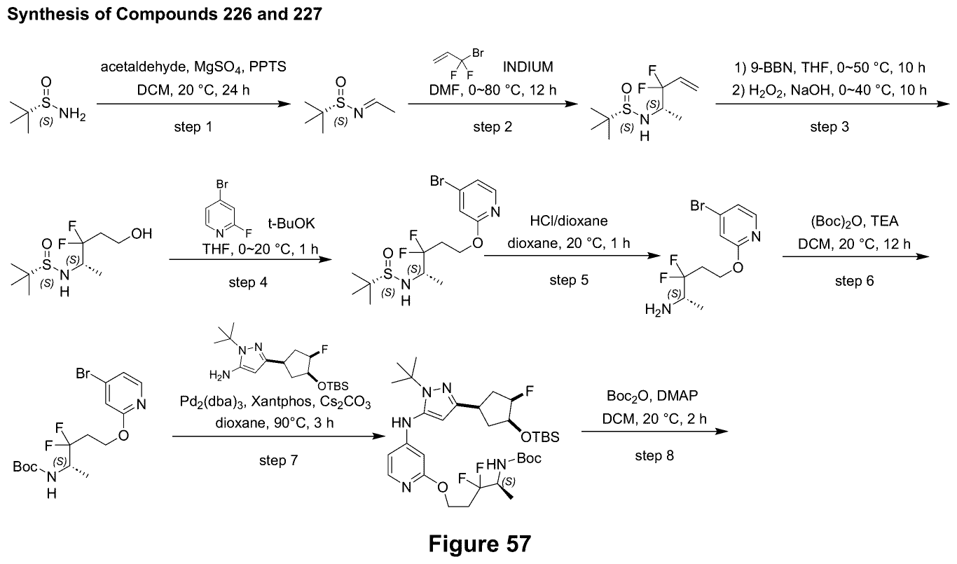

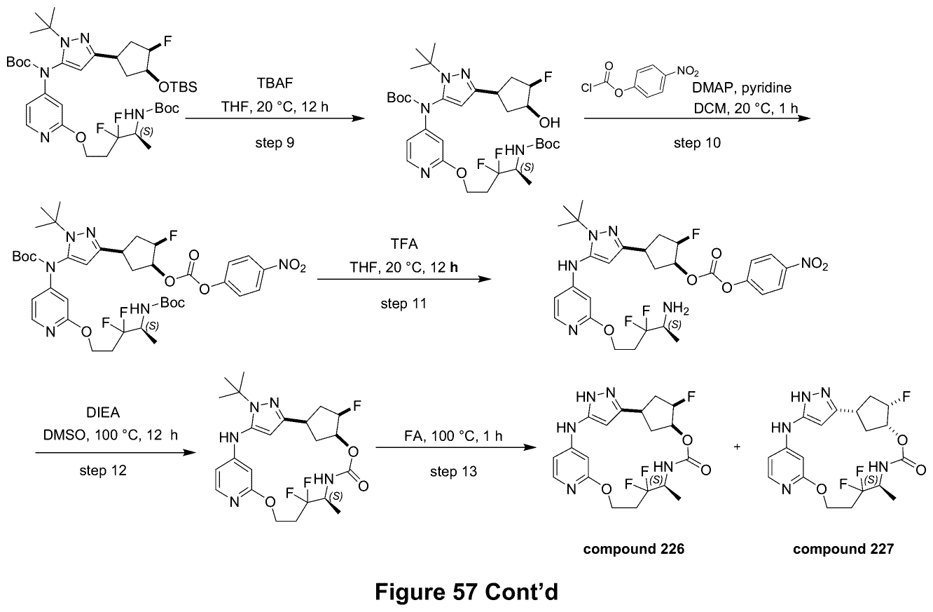

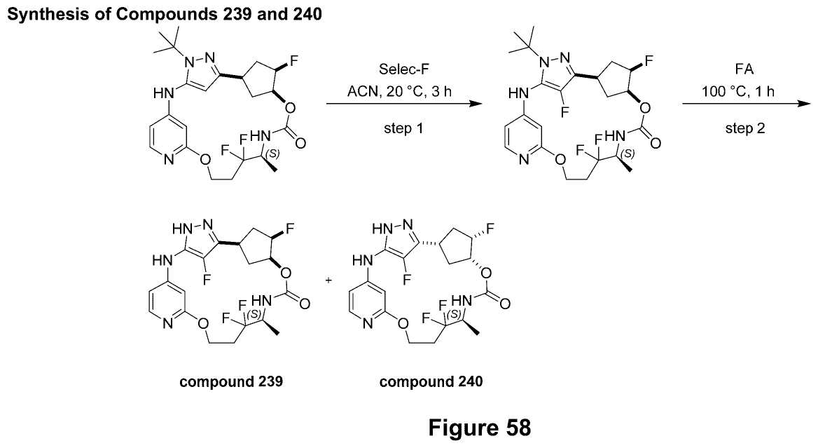

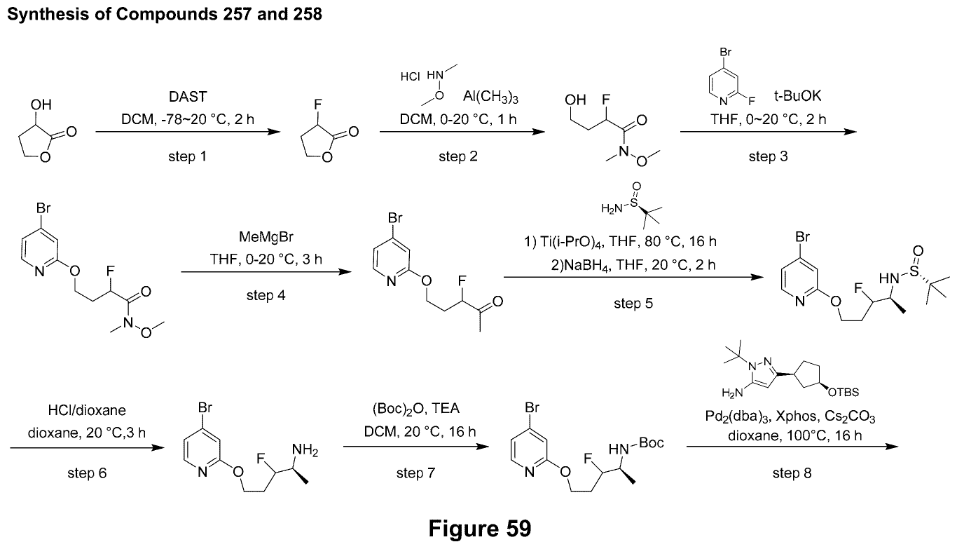

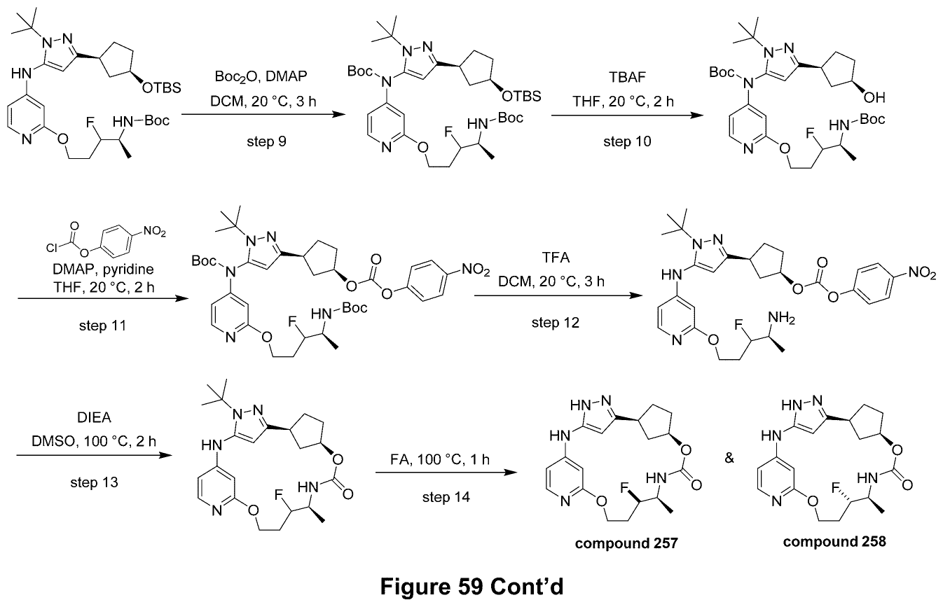

### DEFINITIONS

As used herein, “cancer” refers to diseases, disorders, and conditions that involve abnormal cell growth with the potential to invade or spread to other parts of the body. Exemplary cancers include, but are not limited to, breast cancer, ovarian cancer, uterine cancer, endometrial cancer, cervical cancer, bladder cancer, prostate cancer, lung cancer, stomach cancer, esophageal cancer, colorectal cancer, small bowel cancer, pancreatic cancer, liver cancer, kidney cancer, head and neck cancer, skin cancer, bone cancer, thyroid cancer, peritoneal cancer, and brain cancer.

“Subject” refers to an animal, such as a mammal, that has been or will be the object of treatment, observation, or experiment. The methods described herein may be useful for both human therapy and veterinary applications. In one embodiment, the subject is a human.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

A dash (“-”) that is not between two letters or symbols is used to indicate a point of attachment for a substituent. For example, —CN is attached through the carbon atom.

By “optional” or “optionally” it is meant that the subsequently described event or circumstance may or may not occur, and that the description includes instances where the event or circumstance occurs and instances in which is does not. It will be understood by those skilled in the art, with respect to any group containing one or more substituents, that such groups are not intended to introduce any substitution or substitution patterns that are sterically impractical, synthetically non-feasible and/or inherently unstable.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkenyl” as used herein refers to an unsaturated, two-carbon group having a carbon-carbon double bond, referred to herein as C₂-alkenyl.

The term “alkoxy” as used herein refers to an alkyl or cycloalkyl covalently bonded to an oxygen atom.

The term “alkyl” as used herein refers to a saturated straight or branched hydrocarbon, such as a straight or branched group of 1-8 carbon atoms, referred to herein as (C₁-C₈)alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkynyl” as used herein refers to an unsaturated, two-carbon group having a carbon-carbon triple bond, referred to herein as C₂-alkynyl.

The term “aryl” as used herein refers to a mono-, bi-, or other multi-carbocyclic, aromatic ring system with 5 to 14 ring atoms. The aryl group can optionally be fused to one or more rings selected from aryls, cycloalkyls, heteroaryls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms, referred to herein as “C₆-aryl.”

The term “cycloalkyl” as used herein refers to a saturated or unsaturated cyclic, bicyclic, or bridged bicyclic hydrocarbon group of 3-16 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The terms “halo” or “halogen” as used herein refer to —F, —Cl, —Br, and/or —I.

The term “haloalkyl group” as used herein refers to an alkyl group substituted with one or more halogen atoms.

The term “heteroaryl” as used herein refers to a mono-, bi-, or multi-cyclic, aromatic ring system containing one or more heteroatoms, for example 1-4 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxyl, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C₂-C₅)heteroaryl.” In some embodiments, a heteroaryl contains 5 to 10 ring atoms, 1 to 4 of which are heteroatoms selected from N, O, and S. In some embodiments, a heteroaryl contains 5 to 8 ring atoms, 1 to 4 of which are heteroatoms selected from N, O, and S.

The terms “heterocycle,” “heterocyclyl,” or “heterocyclic” as used herein each refer to a saturated or unsaturated 3- to 18-membered ring containing one, two, three, or four heteroatoms independently selected from nitrogen, oxygen, phosphorus, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone.

Heterocycles also include bicyclic, tricyclic, and tetracyclic groups in which any of the above heterocyclic rings is fused to one or two rings independently selected from aryls, cycloalkyls, and heterocycles. Heterocycles also include bridged and spiro-fused cyclic structures which may or may not contain heteroatoms. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl. In some embodiments, a heterocycle contains 5 to 10 ring atoms, 1 to 4 of which are heteroatoms selected from N, O, and S. In some embodiments, a heterocycle contains 5 to 8 ring atoms, 1 to 4 of which are heteroatoms selected from N, O, and S.

The terms “hydroxy” and “hydroxyl” as used herein refer to —OH.

The term “oxo” as used herein refers to a double bond to an oxygen atom (i.e., ═O). For example, when two geminal groups on a carbon atom are “taken together to form an oxo”, then a carbonyl (i.e., C═O) is formed.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. A “free base” form of a compound, for example, does not contain an ionically bonded salt.

The phrase “and pharmaceutically acceptable salts and deuterated derivatives thereof” is used interchangeably with “and pharmaceutically acceptable salts thereof and deuterated derivatives of any of the forgoing” in reference to one or more compounds or formulae of the disclosure. These phrases are intended to encompass pharmaceutically acceptable salts of any one of the referenced compounds, deuterated derivatives of any one of the referenced compounds, and pharmaceutically acceptable salts of those deuterated derivatives.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Triethiodide |
| Benzenesulfonate | Isethionate | Benzathine |
| Benzoate | Lactate | Chloroprocaine |
| Bicarbonate | Lactobionate | Choline |
| Bitartrate | Malate | Diethanolamine |
| Bromide | Maleate | Ethylenediamine |
| Calcium edetate | Mandelate | Meglumine |
| Camsylate | Mesylate | Procaine |
| Carbonate | Methylbromide | Aluminum |
| Chloride | Methylnitrate | Calcium |
| Citrate | Methylsulfate | Lithium |
| Dihydrochloride | Mucate | Magnesium |
| Edetate | Napsylate | Potassium |
| Edisylate | Nitrate | Sodium |
| Estolate | Pamoate (Embonate) | Zinc |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange.

Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.).

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

The present disclosure includes within its scope all possible tautomers. Furthermore, the present disclosure includes in its scope both the individual tautomers and any mixtures thereof. Each compound disclosed herein includes within its scope all possible tautomeric forms. Furthermore, each compound disclosed herein includes within its scope both the individual tautomeric forms and any mixtures thereof. With respect to the methods, uses, and compositions of the present application, reference to a compound or compounds is intended to encompass that compound in each of its possible isomeric forms and mixtures thereof. Where a compound of the present application is depicted in one tautomeric form, that depicted structure is intended to encompass all other tautomeric forms. As a non-limiting example, compounds illustrated in Table 2 below may contain a pyrazole ring drawn as

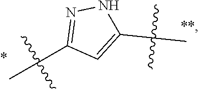 [SMILES: *c1cc(*)[nH]n1]

but some embodiments of the disclosure include compounds with the tautomer

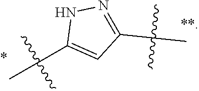 [SMILES: *c1cc(*)[nH]n1]

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of the present disclosure. The present disclosure encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Additionally, unless otherwise stated, structures described herein are also meant to include compounds that differ only in the presence of one or more isotopically enriched atoms. For example, compounds having the present structures except for the replacement of hydrogen by deuterium (²H) or tritium (³H), or the replacement of a carbon by a ¹³C- or ¹⁴C-carbon atom are within the scope of this disclosure. Such compounds may be useful as, for example, analytical tools, probes in biological assays, or therapeutic agents.

### Compounds

In some embodiments, the present disclosure provides a compound represented by Formula (I), or a tautomer, a stereoisomer or a mixture of stereoisomers, a pharmaceutically acceptable salt, a hydrate, or a deuterated derivative thereof:

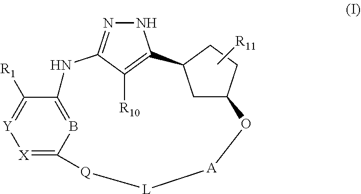

wherein:
A is a substituted or unsubstituted amide or a 5-12 membered aromatic ring, wherein the 5-12 membered aromatic ring optionally includes one or more heteroatoms and is optionally substituted with 1, 2, 3, or 4 R_{A1}, wherein each R_{A1} is independently selected from hydrogen, —C(═O)R_{A2}, C₁-C₅ alkyl, C₂-C₅ alkenyl, C₂-C₅ alkynyl, halo, C₁-C₅ haloalkyl, C₁-C₅ alkoxy, and —CN, and wherein each R_{A2} is independently selected from hydrogen, hydroxyl, C₁-C₅ alkyl, C₁-C₅ alkoxy, C₁-C₅ haloalkyl, C₂-C₅ alkenyl, and C₂-C₅ alkynyl;
B is selected from N, N⁺—O⁻, and CR₀, wherein R₀ is selected from hydrogen, halo, —CN, C₁-C₅ alkyl, C₁-C₅ haloalkyl, C₁-C₅ alkoxy, and C₃-C₅ cycloalkyl;
R₁ is selected from hydrogen, halo, —CN, and C₁-C₅ alkyl, or R₁ and Y together with the carbon atom to which they are attached form a 5-6 membered aromatic ring, wherein the aromatic ring optionally includes 1 to 3 heteroatoms;
X and Y are independently N, N⁺—O⁻, or CR₂, wherein each R₂ is independently selected from hydrogen, haloalkyl, C₁-C₅ alkyl, C₁-C₅ alkoxy, and C₃-C₅ cycloalkyl;
Q is absent or selected from —W—C(R₃)(R₄)—, wherein R₃ and R₄ are independently selected from hydrogen, C₁-C₅ alkyl, C₁-C₅ haloalkyl, and halo, or R₃ and R₄ together with the carbon atom to which they are attached form a carbonyl group or a 3-6 membered cycloalkyl, and wherein W is absent or selected from alkylene, —C(═O)—R₆—, —O—, —R₆—O—, —O—R₆—, —N(R₇)—, —NH—C(═O)—, —C(═O)—N(R₆)—, —S—, —S(═O)₂—, —P(R₇)₂—, 3-6 membered cycloalkyl, and 3-6 membered heterocycle, wherein the alkylene, 3-6 membered cycloalkyl, and 3-6 membered heterocycle are optionally substituted with 1, 2, 3, 4, or 5 R_{B}, wherein each R₆ is independently selected from C₁-C₅ alkylene, 3-6 membered heterocycle, and 3-6 membered cycloalkyl, wherein each R₇ is independently selected from hydrogen, oxo, and C₁-C₅ alkyl, and wherein each R_{B} is independently selected from hydrogen, oxo, halo, and C₁-C₅ alkyl;
L is absent or a linker of 1 to 10 carbon atoms in length, wherein one or more carbon atoms are optionally substituted with oxo, C₁-C₅ haloalkyl, halo, deuterium, or C₁-C₅ alkyl, and wherein one or more carbon atoms are optionally replaced by a 3-6-membered bridged cycloalkyl, a 3-7 membered fused cycloalkyl, alkenyl, —O—, —N(R₈)—, a 3-6-membered cycloalkyl, or a 3-6-membered heterocycle, wherein the 3-6-membered heterocycle, the 3-6-membered cycloalkyl, and the 3-6-membered bridged cycloalkyl are optionally substituted with 1, 2, 3, or 4 R₉, wherein each R₈ is independently selected from hydrogen and C₁-C₅ alkyl, and wherein each R₉ is independently selected from hydrogen, oxo, and C₁-C₅ alkyl; and
R₁₀ and R₁₁ are independently selected from hydrogen, halo, and C₁-C₅ alkyl;
with the proviso that the compound of Formula (I) is not the following:
(1¹S,1³R,Z)-2¹H-12-oxa-3,6,10-triaza-4(4,2)-pyridina-2(3,5)-pyrazola-1(1,3)-cyclopentanacyclododecaphane-5,11-dione,
(1¹S,1³R,Z)-2¹H-12-oxa-3,6,10-triaza-4(3,5)-pyridina-2(5,3)-pyrazola-1(1,3)-cyclopentanacyclododecaphane-5,11-dione,
(1¹S,1³R,Z)-2¹H-12-oxa-3,6,10-triaza-2(5,3)-pyrazola-4(1,3)-benzena-1(1,3)-cyclopentanacyclododecaphane-5,11-dione, or
(1¹S,1³R,Z)-2¹H-13-oxa-3,6,11-triaza-2(5,3)-pyrazola-4(1,3)-benzena-1(1,3)-cyclopentanacyclotridecaphane-5,12-dione.

In some embodiments, disclosed is a compound of Formula (I) selected from compounds of Formula (II), Formula (III), Formula (IV), and Formula (V):

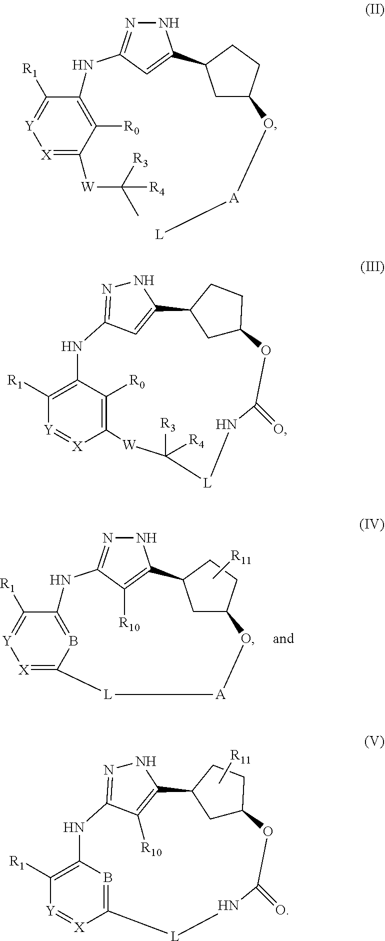

In some embodiments, disclosed is a compound of Formula (I) selected from compounds of Formula (II):

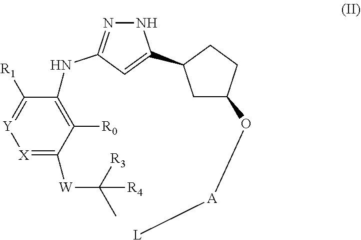

wherein:
A is a substituted or unsubstituted amide or a 5-12 membered aromatic ring, wherein the 5-12 membered aromatic ring optionally includes one or more heteroatoms and is optionally substituted with 1, 2, 3, or 4 R_{A1}, wherein each R_{A1} is independently selected from hydrogen, —C(═O)R_{A2}, C₁-C₅ alkyl, C₂-C₅ alkenyl, C₂-C₅ alkynyl, halo, C₁-C₅ haloalkyl, C₁-C₅ alkoxy, and —CN, and wherein each R_{A2} is independently selected from hydrogen, hydroxyl, C₁-C₅ alkyl, C₁-C₅ alkoxy, C₁-C₅ haloalkyl, C₂-C₅ alkenyl, and C₂-C₅ alkynyl;
R₀ is selected from hydrogen, halo, —CN, and C₁-C₅ alkyl;
R₁ is selected from hydrogen, halo, —CN, and C₁-C₅ alkyl;
R₃ and R₄ are independently selected from hydrogen, C₁-C₅ alkyl, C₁-C₅ haloalkyl, and halo, or R₃ and R₄ together with the carbon atom to which they are attached form a carbonyl group or a 3-6 membered cycloalkyl;
X and Y are independently N or CR₂, wherein each R₂ is independently selected from hydrogen, haloalkyl, C₁-C₅ alkyl, C₁-C₅ alkoxy, and C₃-C₅ cycloalkyl;
W is absent or selected from alkylene, —C(═O)—R₆—, —O—, —R₆—O—, —O—R₆—, —N(R₇)—, —NH—C(═O)—; —C(═O)—N(R₆)—, —S—, —S(═O)₂—, —P(R₇)₂—, 3-6 membered cycloalkyl, and 3-6 membered heterocycle, wherein the alkylene, 3-6 membered cycloalkyl, and 3-6 membered heterocycle are optionally substituted with 1, 2, 3, 4, or 5 R_{B}, wherein each R₆ is independently selected from C₁-C₅ alkylene, 3-6 membered heterocycle, and 3-6 membered cycloalkyl, wherein each R₇ is independently selected from hydrogen, oxo, and C₁-C₅ alkyl, and wherein each R_{B} is independently selected from hydrogen, oxo, halo, and C₁-C₅ alkyl; and
L is absent or a linker of 1 to 10 carbon atoms in length, wherein one or more carbon atoms are optionally substituted with oxo, C₁-C₅ haloalkyl, halo, or C₁-C₅ alkyl, and wherein one or more carbon atoms are optionally replaced by a 3-6-membered bridged cycloalkyl, 3-7 membered fused cycloalkyl, alkenyl, —O—, —N(R₈)—, 3-6-membered cycloalkyl, or 3-6-membered heterocycle, wherein the 3-6-membered heterocycle, the 3-6-membered cycloalkyl, and the 3-6-membered bridged cycloalkyl are optionally substituted with 1, 2, 3, or 4 R₉, wherein each R₈ is independently selected from hydrogen and C₁-C₅ alkyl, and wherein each R₉ is independently selected from hydrogen, oxo, and C₁-C₅ alkyl.

In some embodiments, disclosed is a compound of Formula (I) selected from compounds of Formula (III):

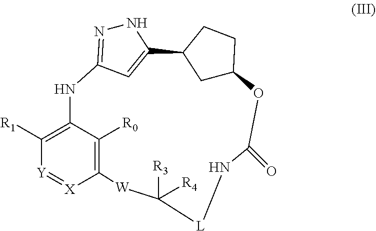

wherein:
R₀ is selected from hydrogen, halo, —CN, and C₁-C₅ alkyl;
R₁ is selected from hydrogen, halo, —CN, and C₁-C₅ alkyl;
R₃ and R₄ are independently selected from hydrogen, C₁-C₅ alkyl, C₁-C₅ haloalkyl, and halo, or R₃ and R₄ together with the carbon atom to which they are attached form a carbonyl group, or a 3-6 membered cycloalkyl;
X and Y are independently N or CR₂, wherein each R₂ is independently selected from hydrogen, haloalkyl, C₁-C₅ alkyl, C₁-C₅ alkoxy, and C₃-C₅ cycloalkyl;
W is absent or selected from alkylene, —C(═O)—R₆—, —O—, —R₆—O—, —O—R₆—, —N(R₇)—, —NH—C(═O)—, —C(═O)—N(R₆)—, —S—, —S(═O)₂—, —P(R₇)₂—, 3-6 membered cycloalkyl, and 3-6 membered heterocycle, wherein the alkylene, 3-6 membered cycloalkyl, and 3-6 membered heterocycle are optionally substituted with 1, 2, 3, 4, or 5 R_{B}, wherein each R₆ is independently selected from C₁-C₅ alkylene, 3-6 membered heterocycle, and 3-6 membered cycloalkyl, wherein each R₇ is independently selected from hydrogen, oxo, and C₁-C₅ alkyl, and wherein each R_{B} is independently selected from hydrogen, oxo, halo, and C₁-C₅ alkyl; and
L is absent or a linker of 1 to 10 carbon atoms in length, wherein one or more carbon atoms are optionally substituted with oxo, C₁-C₅ haloalkyl, halo, or C₁-C₅ alkyl, and wherein one or more carbon atoms are optionally replaced by a 3-6-membered bridged cycloalkyl, a 3-7 membered fused cycloalkyl, alkenyl, —O—, —N(R₈)—, 3-6-membered cycloalkyl, or 3-6-membered heterocycle, wherein the 3-6-membered heterocycle, the 3-6-membered cycloalkyl, and the 3-6-membered bridged cycloalkyl are optionally substituted with 1, 2, 3, or 4 R₉, wherein each R₈ is independently selected from hydrogen and C₁-C₅ alkyl, and wherein each R₉ is independently selected from hydrogen, oxo, and C₁-C₅ alkyl.

In some embodiments, disclosed is a compound of Formula (I) selected from compounds of Formula (IV):

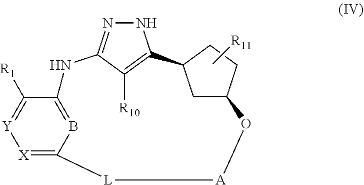

wherein:
R₁₀ and R₁₁ are independently selected from hydrogen, halo, and C₁-C₅ alkyl;
A is a substituted or unsubstituted amide or a 5-12 membered aromatic ring, wherein the 5-12 membered aromatic ring optionally includes one or more heteroatoms and is optionally substituted with 1, 2, 3, or 4 R_{A1}, wherein each R_{A1} is independently selected from hydrogen, —C(═O)—R_{A2}, C₁-C₅ alkyl, C₂-C₅ alkenyl, C₂-C₅ alkynyl, halo, C₁-C₅ haloalkyl, C₁-C₅ alkoxy, and —CN, and wherein each R_{A2} is independently selected from hydrogen, hydroxyl, C₁-C₅ alkyl, C₁-C₅ alkoxy, C₁-C₅ haloalkyl, C₂-C₅ alkenyl, and C₂-C₅ alkynyl;
B is selected from N, N⁺—O⁻, and CR₀, wherein R₀ is selected from hydrogen, halo, —CN, C₁-C₅ alkyl, C₁-C₅ haloalkyl, C₁-C₅ alkoxy, and C₃-C₅ cycloalkyl;
R₁ is selected from hydrogen, halo, —CN, and C₁-C₅ alkyl, or R₁ and Y together with the carbon atom to which they are attached form a 5-6 membered aromatic ring, wherein the aromatic ring optionally includes 1 to 3 heteroatoms;
X and Y are independently N, N⁺—O⁻, or CR₂, wherein each R₂ is independently selected from hydrogen, haloalkyl, C₁-C₅ alkyl, C₁-C₅ alkoxy, and C₃-C₅ cycloalkyl; and
L is a linker of 1 to 10 carbon atoms in length, wherein one or more carbon atoms are optionally substituted with oxo, C₁-C₅ haloalkyl, halo, deuterium, or C₁-C₅ alkyl, and wherein one or more carbon atoms are optionally replaced by —C(═O)—R₆—, —O—, —R₆₀—, —OR₆—, —N(R₇)—, —NH—C(═O)—, —C(═O)—N(R₆)—, —S—, —S(═O)₂—, —P(R₇)₂—, a 3-6-membered bridged cycloalkyl, a 3-7 membered fused cycloalkyl, alkenyl, —O—, —N(R₈)—, 3-6 membered cycloalkyl, or 3-6-membered heterocycle, wherein the 3-6-membered heterocycle, the 3-6 membered cycloalkyl, and the 3-6 membered bridged cycloalkyl are optionally substituted with 1, 2, 3, 4, or 5 R₉, wherein each R₆ is independently selected from hydrogen, C₁-C₅ alkylene, 3-6 membered heterocycle, and 3-6 membered cycloalkyl, wherein each R₇ is independently selected from hydrogen and C₁-C₅ alkyl, wherein each R₈ is independently selected from hydrogen and C₁-C₅ alkyl, and wherein each R₉ is independently selected from hydrogen, oxo, halo, and C₁-C₅ alkyl.

In some embodiments, disclosed is a compound of Formula (I) selected from compounds of Formula (V):

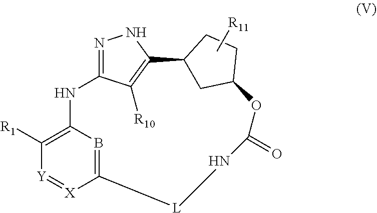

wherein:
R₁₀ and R₁₁ are independently selected from hydrogen, halo, and C₁-C₅ alkyl; B is selected from N, N⁺—O⁻, and CR₀, wherein R₀ is selected from hydrogen, halo, —CN, C₁-C₅ alkyl, C₁-C₅ haloalkyl, C₁-C₅ alkoxy, and C₃-C₅ cycloalkyl;
R₁ is selected from hydrogen, halo, —CN, and C₁-C₅ alkyl, or R₁ and Y together with the carbon atom to which they are attached form a 5-6 membered aromatic ring, wherein the aromatic ring optionally includes 1 to 3 heteroatoms;
X and Y are independently N, N⁺—O⁻, or CR₂, wherein each R₂ is independently selected from hydrogen, haloalkyl, C₁-C₅ alkyl, C₁-C₅ alkoxy, and C₃-C₅ cycloalkyl; and
L is a linker of 1 to 10 carbon atoms in length, wherein one or more carbon atoms are optionally substituted with oxo, C₁-C₅ haloalkyl, halo, deuterium, or C₁-C₅ alkyl, and wherein one or more carbon atoms are optionally replaced by —C(═O)—R₆—, —O—, —R₆—O—, —O—R₆—, —N(R₇)—, —NH—C(═O)—, —C(═O)—N(R₆)—, —S—, —S(═O)₂—, —P(R₇)₂—, a 3-6-membered bridged cycloalkyl, a 3-7 membered fused cycloalkyl, alkenyl, —O—, —N(R₈)—, 3-6 membered cycloalkyl, or 3-6-membered heterocycle, wherein the 3-6-membered heterocycle, the 3-6 membered cycloalkyl, and the 3-6 membered bridged cycloalkyl are optionally substituted with 1, 2, 3, 4, or 5 R₉, wherein each R₆ is independently selected from hydrogen, C₁-C₅ alkylene, 3-6 membered heterocycle, and 3-6 membered cycloalkyl, wherein each R₇ is independently selected from hydrogen and C₁-C₅ alkyl, wherein each R₈ is independently selected from hydrogen and C₁-C₅ alkyl, and wherein each R₉ is independently selected from hydrogen, oxo, halo, and C₁-C₅ alkyl.

In some embodiments, disclosed is a compound of Formula (I), or a tautomer, a stereoisomer or a mixture of stereoisomers, a pharmaceutically acceptable salt, a hydrate, or a deuterated derivative thereof:

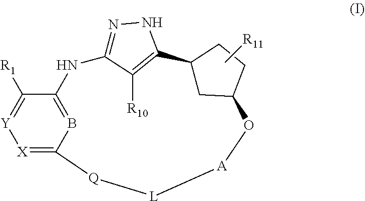

wherein:
A is substituted or unsubstituted amide;
B is selected from N and CR₀, wherein R₀ is selected from hydrogen, halo, and C₁-C₅ alkyl;
R₁ is hydrogen;
X and Y are independently N or CR₂, wherein each R₂ is independently selected from hydrogen, haloalkyl, and C₁-C₅ alkyl;
Q is absent or selected from —W—C(R₃)(R₄)—, wherein R₃ and R₄ are independently selected from hydrogen, halo, C₁-C₅ alkyl, and deuterium, and wherein W is absent or —O—;
L is a linker of 1 to 10 carbon atoms in length, wherein one or more carbon atoms are optionally substituted with halo, deuterium, or C₁-C₅ alkyl, and wherein one or more carbon atoms are optionally replaced by —O— or a 3-6 membered cycloalkyl; and
R₁₀ and R₁₁ are independently selected from hydrogen, halo, and C₁-C₅ alkyl.

In some embodiments, disclosed is a compound of Formula (I), or a tautomer, a stereoisomer or a mixture of stereoisomers, a pharmaceutically acceptable salt, a hydrate, or a deuterated derivative thereof:

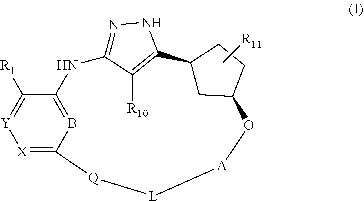

wherein:
A is substituted or unsubstituted amide;
B is CR₀, wherein R₀ is hydrogen;
X and Y are independently N or CR₂, wherein R₂ is independently selected from hydrogen, haloalkyl, C₁-C₅ alkyl, and C₃-C₅ cycloalkyl;
Q is absent or —W—C(R₃)(R₄)—, wherein W is —O—, and wherein R₃ and R₄ are independently selected from hydrogen and C₁-C₅ alkyl;
L is a linker of 1 to 10 carbon atoms in length, wherein one or more carbon atoms are optionally substituted with C₁-C₅ alkyl, and wherein one or more carbon atoms are optionally replaced by, —O—, a 3-6 membered cycloalkyl; and
R₁₀ and R₁₁ are each hydrogen.

In some embodiments, A is

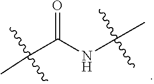 [SMILES: *NC(*)=O]

In some embodiments, A is selected from a 5-membered aromatic ring and a 6-membered aromatic ring. In some embodiments, A is selected from a 5-membered aromatic ring and a 6-membered aromatic ring, wherein the 5-membered aromatic ring and the 6-membered aromatic ring include one or more heteroatoms. In some embodiments, A is selected from a 5-membered aromatic ring, wherein the 5-membered aromatic ring includes one or more heteroatoms. In some embodiments, A is selected from a 5-membered aromatic ring, wherein the 5-membered aromatic ring includes one nitrogen atom. In some embodiments, A is selected from a 5-membered aromatic ring, wherein the 5-membered aromatic ring includes two nitrogen atoms. In some embodiments, A is selected from a 6-membered aromatic ring, wherein the 6-membered aromatic ring includes one or more heteroatoms. In some embodiments, A is selected from a 6-membered aromatic ring, wherein the 6-membered aromatic ring includes one nitrogen atom. In some embodiments, A is selected from a 6-membered aromatic ring, wherein the 6-membered aromatic ring includes two nitrogen atoms. In some embodiments, A is selected from a 6-membered aromatic ring, wherein the 6-membered aromatic ring includes three nitrogen atoms. In some embodiments, A is selected from a 6-membered aromatic ring, wherein the 6-membered aromatic ring includes four nitrogen atoms.

In some embodiments, A is selected from

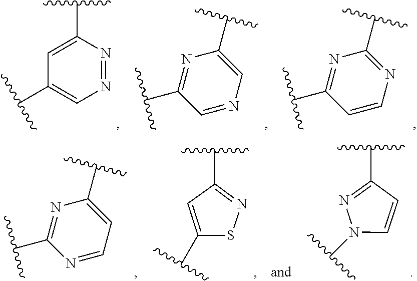

In some embodiments, at least one R_{A1} is C₁-C₅ alkoxy. In some embodiments, at least one R_{A1} is hydrogen. In some embodiments, at least one R_{A1} is —C(═O)—R_{A2}. In some embodiments, at least one R_{A1} is C₁-C₅ alkyl. In some embodiments, at least one R_{A1} is C₂-C₅ alkenyl. In some embodiments, at least one R_{A1} is C₂-C₅ alkynyl. In some embodiments, at least one R_{A1} is halo. In some embodiments, at least one R_{A1} is C₁-C₅ haloalkyl. In some embodiments, at least one R_{A1} is —CN.

In some embodiments, each R_{A1} is C₁-C₅ alkoxy. In some embodiments, each R_{A1} is hydrogen. In some embodiments, each R_{A1} is —C(═O)—R_{A2}. In some embodiments, each R_{A1} is C₁-C₅ alkyl. In some embodiments, each R_{A1} is C₂-C₅ alkenyl. In some embodiments, each R_{A1} is C₂-C₅ alkynyl. In some embodiments, each R_{A1} is halo. In some embodiments, each R_{A1} is C₁-C₅ haloalkyl. In some embodiments, each R_{A1} is —CN.

In some embodiments, at least one R_{A2} is C₁-C₅ alkoxy. In some embodiments, at least one R_{A2} is C₁-C₅ alkyl. In some embodiments, at least one R_{A2} is hydrogen. In some embodiments, at least one R_{A2} is hydroxyl. In some embodiments, at least one R_{A2} is C₁-C₅ haloalkyl. In some embodiments, at least one R_{A2} is C₂-C₅ alkynyl.

In some embodiments, each R_{A2} is C₁-C₅ alkoxy. In some embodiments, each R_{A2} is C₁-C₅ alkyl. In some embodiments, each R_{A2} is hydrogen. In some embodiments, each R_{A2} is hydroxyl. In some embodiments, each R_{A2} is C₁-C₅ haloalkyl. In some embodiments, each R_{A2} is C₂-C₅ alkynyl.

In some embodiments, B is N. In some embodiments, B is N⁺—O⁻. In some embodiments, B is CR₀.

In some embodiments, R₀ is hydrogen. In some embodiments, R₀ is halo. In some embodiments, R₀ is —CN. In some embodiments, R₀ is C₁-C₅ alkyl. In some embodiments, R₀ is C₁-C₅ haloalkyl. In some embodiments, R₀ is C₁-C₅ alkoxy. In some embodiments, R₀ is C₁-C₅ cycloalkyl.

In some embodiments, R₁ is hydrogen. In some embodiments, R₁ is halo. In some embodiments, R₁ is independently selected from F, Cl, I, and Br. In some embodiments, R₁ is C₁-C₅ alkyl. In some embodiments, R₁ is independently selected from —CH₃, —CH₂—CH₃, —CH(CH₃)₂, and —CH₂—CH₂—CH₃.

In some embodiments, R₁ and Y together with the carbon atom to which they are attached form a 5-6 membered aromatic ring. In some embodiments, the 5-6 membered aromatic ring includes 1 to 3 heteroatoms.

In some embodiments, Y is CR₂. In some embodiments, Y is N. In some embodiments, Y is N⁺—O⁻.

In some embodiments, X is CR₂. In some embodiments, X is N. In some embodiments, X is N⁺—O⁻.

In some embodiments, X and Y are each CR₂. In some embodiments, X and Y are each N.

In some embodiments, at least one R₂ is independently selected from hydrogen,

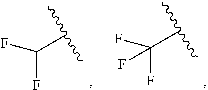

—CH₃, —CH₂—CH₃, —CH(CH₃)₂, —CH₂—CH₂—CH₃,

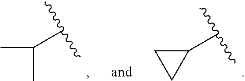

In some embodiments, at least one R₂ is hydrogen,

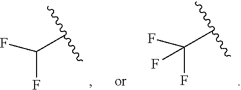

In some embodiments, at least one R₂ is C₁-C₅ alkoxy. In some embodiments, at least one R₂ is a halogen selected from F, Cl, Br, and I.

In some embodiments, each R₂ is independently selected from hydrogen,

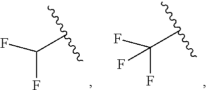

—CH₃, —CH₂—CH₃, —CH(CH₃)₂, —CH₂—CH₂—CH₃,

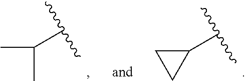

In some embodiments, each R₂ is hydrogen,

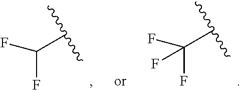

In some embodiments, each R₂ is C₁-C₅ alkoxy. In some embodiments, each R₂ is a halogen selected from F, Cl, Br, and I.

In some embodiments, Q is —W—C(R₃)(R₄)—. In some embodiments, Q is absent.

In some embodiments, R₃ and R₄ are independently selected from hydrogen, deuterium, C₁-C₅ alkyl, C₁-C₅ haloalkyl, and halo. In some embodiments, R₃ and R₄ together with the carbon atom to which they are attached form a carbonyl group or a 3-6 membered cycloalkyl. In some embodiments, R₃ and R₄ are each deuterium. In some embodiments, R₃ and R₄ are independently selected from halo. In some embodiments, R₃ and R₄ are independently selected from F, Br, Cl, and I. In some embodiments, R₃ and R₄ are independently selected from C₁-C₅ alkyl. In some embodiments, R₃ and R₄ are independently selected from —CH₃, —CH₂—CH₃, —CH(CH₃)₂, and —CH₂—CH₂—CH₃.

In some embodiments, W is absent. In some embodiments, W is independently selected from alkylene, —C(═O)—R₆—, —O—, —R₆—O—, —O—R₆—, —N(R₇)—, —NH—C(═O)—; —C(═O)—N(R₆)—, —S(═O)₂—, —P(R₇)₂—, 3-6 membered cycloalkyl, and 3-6 membered heterocycle, wherein the alkylene, 3-6 membered cycloalkyl, and 3-6 membered heterocycle are optionally substituted with 1, 2, 3, 4, or 5 R_{B}.

In some embodiments, W is a 3-6 membered cycloalkyl or a 3-6 membered heterocycle optionally substituted with 1, 2, 3, or 4 R_{B}.

In some embodiments, W is independently selected from

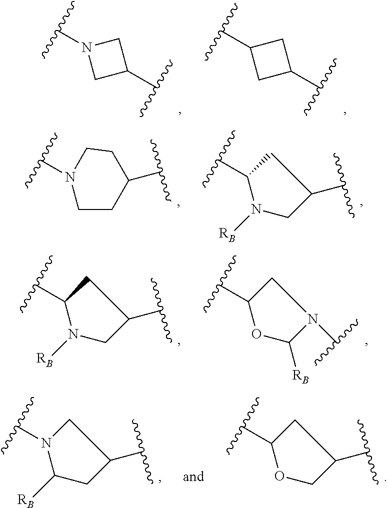

In some embodiments, at least one R₆ is independently selected from —CH₂—, —CH₂—CH₂—, —CH₂—CH₂—CH₂—, and —CH₂—CH₂—CH₂—CH₂—.

In some embodiments, at least one R₆ is independently selected from

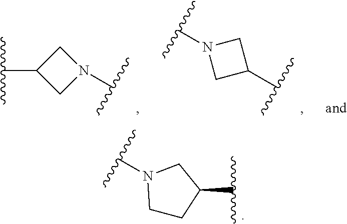 [SMILES: *C1CN(*)C1 ; *C1CN(*)C1 ; *[C@H]1CCN(*)C1]

In some embodiments, each R₆ is independently selected from —CH₂—, —CH₂—CH₂—, —CH₂—CH₂—CH₂—, and —CH₂—CH₂—CH₂—CH₂—.

In some embodiments, each R₆ is independently selected from

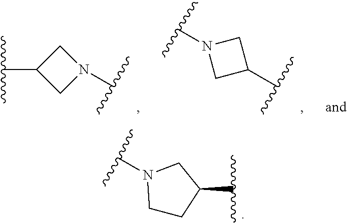 [SMILES: *C1CN(*)C1 ; *C1CN(*)C1 ; *[C@H]1CCN(*)C1]

In some embodiments, at least one R_{B} is independently selected from hydrogen, oxo, —CH₃, —CH₂—CH₃, —CH(CH₃)₂, and —CH₂—CH₂—CH₃. In some embodiments, each R_{B} is independently selected from hydrogen, oxo, —CH₃, —CH₂—CH₃, —CH(CH₃)₂, and —CH₂—CH₂—CH₃.

In some embodiments, L is absent. In some embodiments, L is a linker of 1 to 10 carbon atoms in length. In some embodiments, L is a linker of 1 to 5 carbon atoms in length. In some embodiments, one or more carbon atoms of L are optionally substituted with oxo, C₁-C₅ haloalkyl, halo, deuterium, or C₁-C₅ alkyl. In some embodiments, one or more carbon atoms of L are optionally substituted with C₁-C₅ alkyl, wherein the C₁-C₅ alkyl is independently selected from —CH₃, —CH₂—CH₃, —CH(CH₃)₂, and —CH₂—CH₂—CH₃. In some embodiments, one or more carbon atoms of L are optionally replaced by a 3-6-membered bridged cycloalkyl, a 3-7 membered fused cycloalkyl, alkenyl, —O—, —S—, —S(═O)₂—, —N(R₈), 3-6-membered cycloalkyl, or 3-6-membered heterocycle, and wherein the 3-6-membered heterocycle, the 3-6-membered cycloalkyl, and the 3-6 membered bridged cycloalkyl are optionally substituted with 1, 2, 3, or 4 R₉

In some embodiments, at least one R₈ is independently selected from hydrogen, —CH₃, —CH₂—CH₃, —CH(CH₃)₂, and —CH₂—CH₂—CH₃.

In some embodiments, each R₈ is independently selected from hydrogen, —CH₃, —CH₂—CH₃, —CH(CH₃)₂, and —CH₂—CH₂—CH₃.

In some embodiments, at least one R₉ is independently selected from hydrogen, oxo, —CH₃, —CH₂—CH₃, —CH(CH₃)₂, and —CH₂—CH₂—CH₃.

In some embodiments, each R₉ is independently selected from hydrogen, oxo, —CH₃, —CH₂—CH₃, —CH(CH₃)₂, and —CH₂—CH₂—CH₃.

In some embodiments, L is a linker of 1 to 5 carbon atoms in length, and wherein the one or more carbon atoms are optionally replaced by a 3-6 membered bridged cycloalkyl, a 3-6 membered cycloalkyl, or a 3-6 membered heterocycle. In some embodiments, the 3-6 membered bridged cycloalkyl, 3-6 membered cycloalkyl, and the 3-6 membered heterocycle are independently selected from

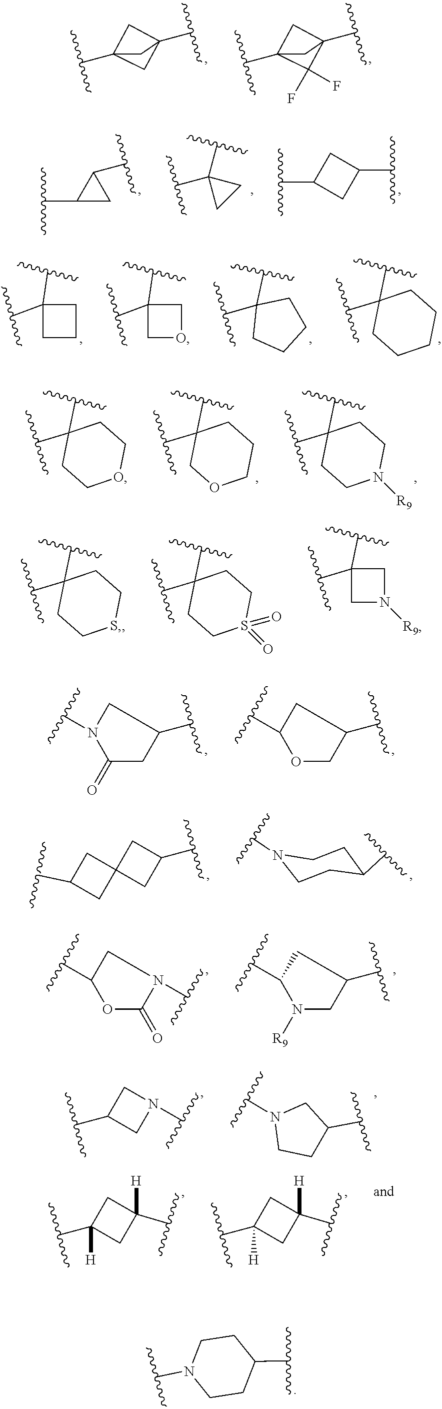

In some embodiments, the 3-6 membered bridged cycloalkyl, 3-6 membered cycloalkyl, and the 3-6 membered heterocycle are independently selected from

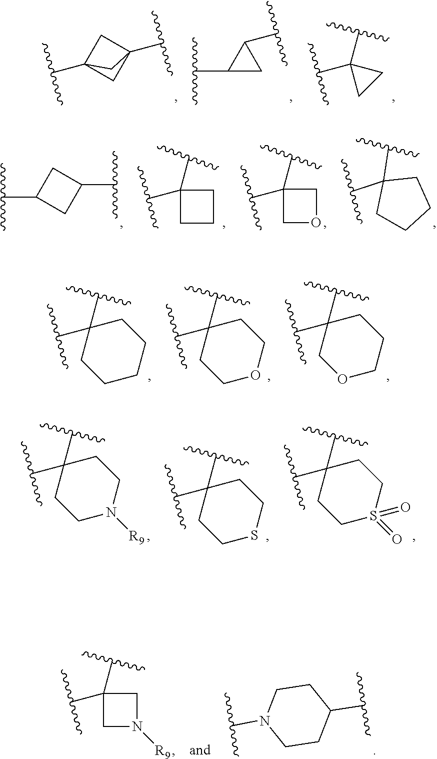

In some embodiments, L is selected from

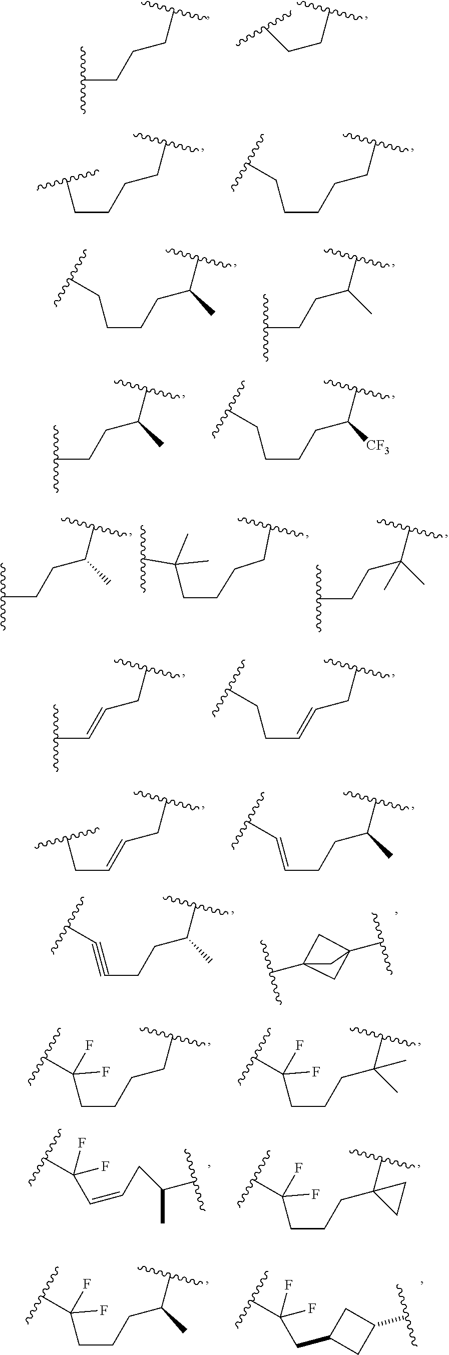

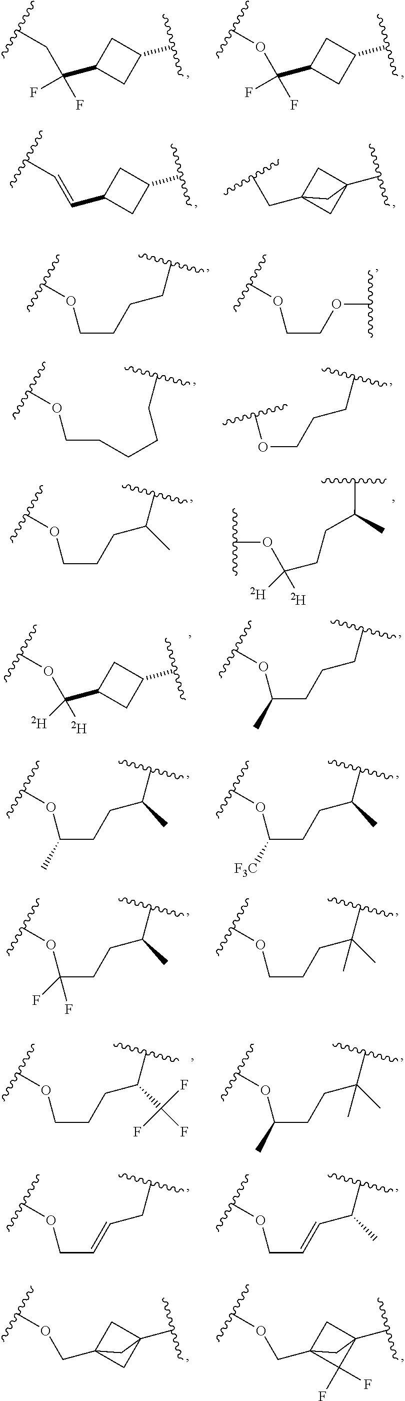

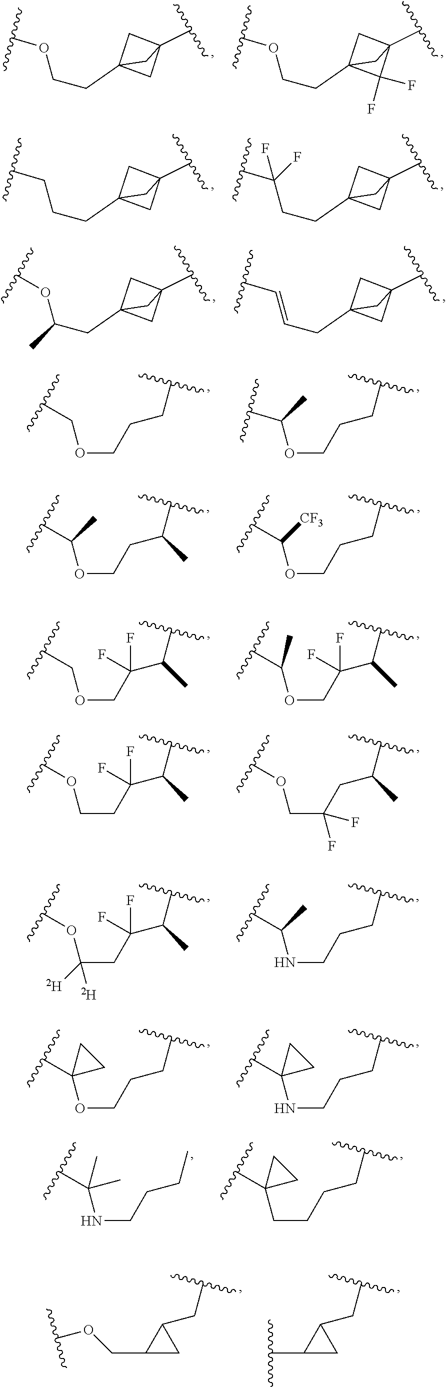

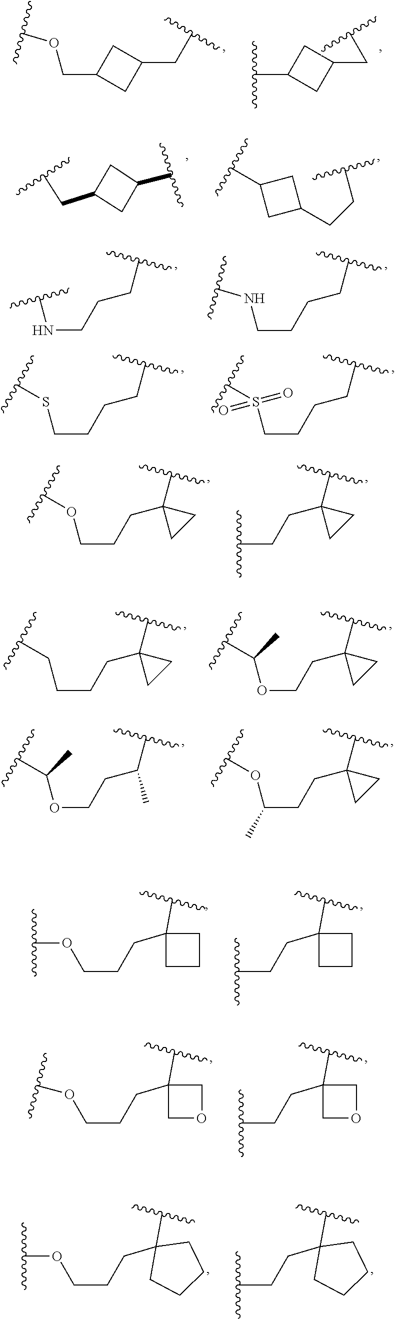

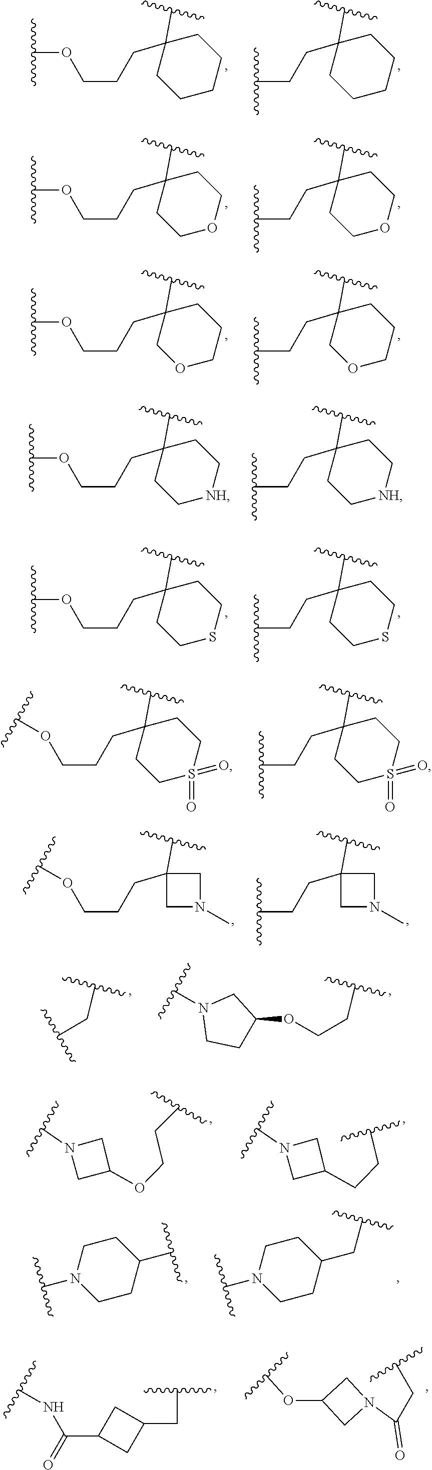

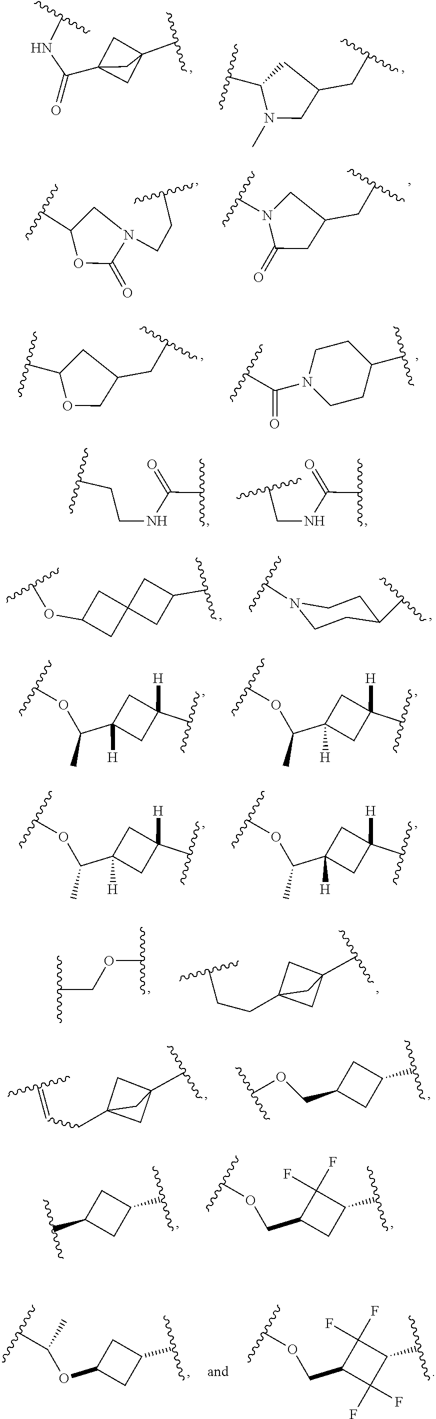

In some embodiments, L is selected from

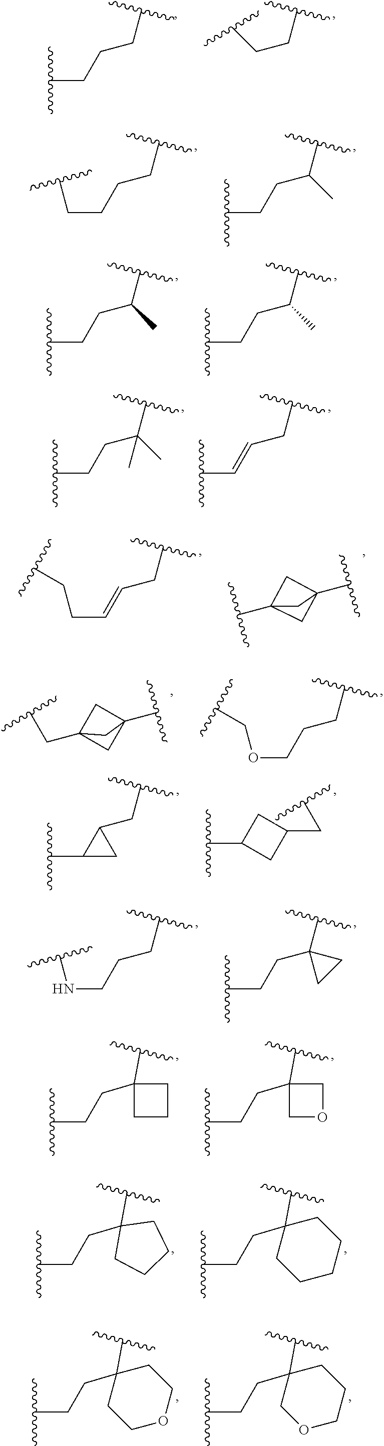

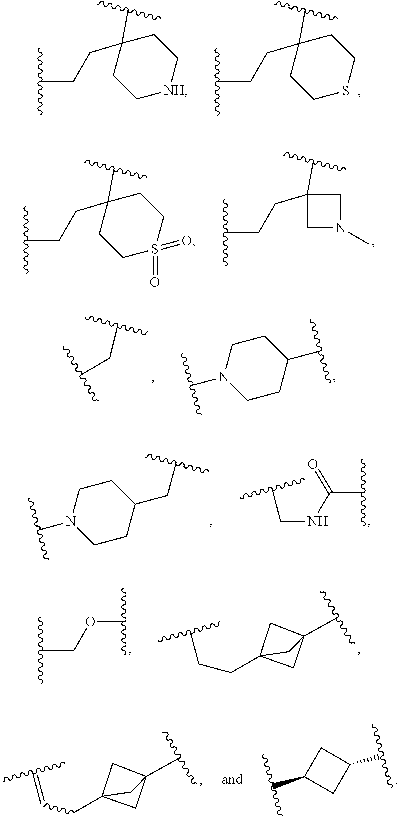

In some embodiments, provided herein is a compound chosen from the compounds listed in Table 2 or a tautomer, a stereoisomer or a mixture of stereoisomers, a pharmaceutically acceptable salt, a hydrate, or a deuterated derivative thereof.

TABLE 2
Exemplary Compounds of the Present Disclosure

| Compound | | |
|---|---|---|
| No. | Chemical Structure | IUPAC Nomenclature |
| 1 | 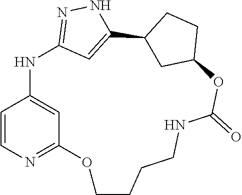 [SMILES: O=C1NCCCCOc2cc(ccn2)Nc2cc([nH]n2)[C@H]2CC[C@H](C2)O1] | (1¹S,1³R,Z)-2¹H-5,12-dioxa-3,10- diaza-4(4,2)-pyridina-2(3,5)- pyrazola-1(1,3)- cyclopentanacyclododecaphan-11- one |
| 2 | 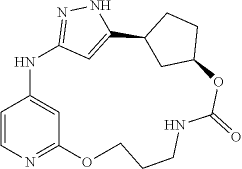 [SMILES: O=C1NCCCOc2cc(ccn2)Nc2cc([nH]n2)[C@H]2CC[C@H](C2)O1] | (1¹S,1³R,Z)-2¹H-5,11-dioxa-3,9- diaza-4(4,2)-pyridina-2(3,5)- pyrazola-1(1,3)- cyclopentanacycloundecaphan-10- one |
| 3 | 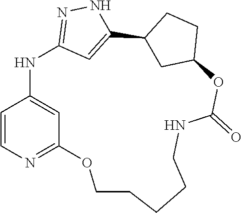 [SMILES: O=C1NCCCCCOc2cc(ccn2)Nc2cc([nH]n2)[C@H]2CC[C@H](C2)O1] | (1¹S,1³R,Z)-2¹H-5,13-dioxa-3,11- diaza-4(4,2)-pyridina-2(3,5)- pyrazola-1(1,3)- cyclopentanacyclotridecaphan-12- one |
| 4 | 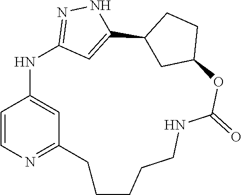 [SMILES: O=C1NCCCCCc2cc(ccn2)Nc2cc([nH]n2)[C@H]2CC[C@H](C2)O1] | (1¹S,1³R,Z)-2¹H-12-oxa-3,10-diaza- 4(4,2)-pyridina-2(3,5)-pyrazola- 1(1,3)- cyclopentanacyclododecaphan-11- one |
| 5 | 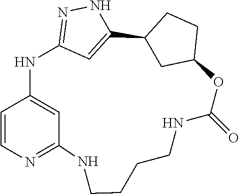 [SMILES: O=C1NCCCCNc2cc(ccn2)Nc2cc([nH]n2)[C@H]2CC[C@H](C2)O1] | (1¹S,1³R,Z)-2¹H-12-oxa-3,5,10- triaza-4(4,2)-pyridina-2(3,5)- pyrazola-1(1,3)- cyclopentanacyclododecaphan-11- one |
| 6 | 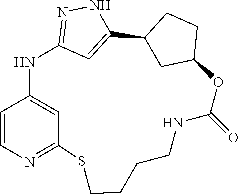 [SMILES: O=C1NCCCCSc2cc(ccn2)Nc2cc([nH]n2)[C@H]2CC[C@H](C2)O1] | (1¹S,1³R,Z)-2¹H-12-oxa-5-thia-3,10- diaza-4(4,2)-pyridina-2(3,5)- pyrazola-1(1,3)- cyclopentanacyclododecaphan-11- one |
| 7 | 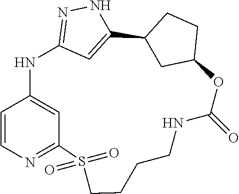 [SMILES: O=C1NCCCCS(=O)(=O)c2cc(ccn2)Nc2cc([nH]n2)[C@H]2CC[C@H](C2)O1] | (1¹S,1³R,Z)-2¹H-12-oxa-5-thia-3,10- diaza-4(4,2)-pyridina-2(3,5)- pyrazola-1(1,3)- cyclopentanacyclododecaphan-11- one 5,5-dioxide |
| 8 | 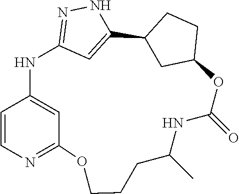 [SMILES: CC1CCCOc2cc(ccn2)Nc2cc([nH]n2)[C@H]2CC[C@H](C2)OC(=O)N1] | (1¹S,1³R,Z)-9-methyl-2¹H-5,12- dioxa-3,10-diaza-4(4,2)-pyridina- 2(3,5)-pyrazola-1(1,3)- cyclopentanacyclododecaphan-11- one |
| 9 | 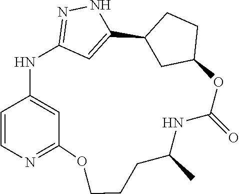 [SMILES: C[C@H]1CCCOc2cc(ccn2)Nc2cc([nH]n2)[C@H]2CC[C@H](C2)OC(=O)N1] | (1¹S,1³R,9S,Z)-9-methyl-2¹H-5,12- dioxa-3,10-diaza-4(4,2)-pyridina- 2(3,5)-pyrazola-1(1,3)- cyclopentanacyclododecaphan-11- one |
| 10 | 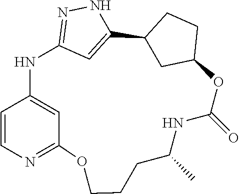 [SMILES: C[C@@H]1CCCOc2cc(ccn2)Nc2cc([nH]n2)[C@H]2CC[C@H](C2)OC(=O)N1] | (1¹S,1³R,9R,Z)-9-methyl-2¹H-5,12- dioxa-3,10-diaza-4(4,2)-pyridina- 2(3,5)-pyrazola-1(1,3)- cyclopentanacyclododecaphan-11- one |
| 11 | 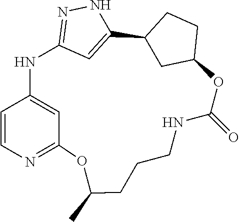 [SMILES: C[C@@H]1CCCNC(=O)O[C@@H]2CC[C@@H](C2)c2cc(n[nH]2)Nc2ccnc(c2)O1] | (1¹S,1³R,6R,Z)-6-methyl-2¹H-5,12- dioxa-3,10-diaza-4(4,2)-pyridina- 2(3,5)-pyrazola-1(1,3)- cyclopentanacyclododecaphan-11- one |
| 12 | 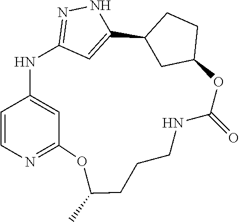 [SMILES: C[C@H]1CCCNC(=O)O[C@@H]2CC[C@@H](C2)c2cc(n[nH]2)Nc2ccnc(c2)O1] | (1¹S,1³R,6S,Z)-6-methyl-2¹H-5,12- dioxa-3,10-diaza-4(4,2)-pyridina- 2(3,5)-pyrazola-1(1,3)- cyclopentanacyclododecaphan-11- one |
| 13 | 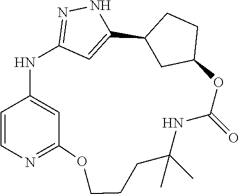 [SMILES: CC1(C)CCCOc2cc(ccn2)Nc2cc([nH]n2)[C@H]2CC[C@H](C2)OC(=O)N1] | (1¹S,1³R,Z)-9,9-dimethyl-2¹H-5,12- dioxa-3,10-diaza-4(4,2)-pyridina- 2(3,5)-pyrazola-1(1,3)- cyclopentanacyclododecaphan-11- one |
| 14 | 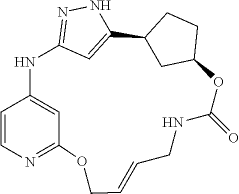 [SMILES: O=C1NC/C=C/COc2cc(ccn2)Nc2cc([nH]n2)[C@H]2CC[C@H](C2)O1] | (1¹S,1³R,24Z,7E)-2¹H-5,12-dioxa- 3,10-diaza-4(4,2)-pyridina-2(3,5)- pyrazola-1(1,3)- cyclopentanacyclododecaphan-7- en-11-one |
| 15 | 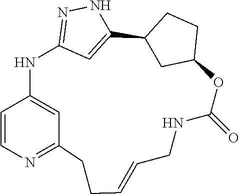 [SMILES: O=C1NC/C=C/CCc2cc(ccn2)Nc2cc([nH]n2)[C@H]2CC[C@H](C2)O1] | (1¹S,1³R,24Z,7E)-2¹H-12-oxa-3,10- diaza-4(4,2)-pyridina-2(3,5)- pyrazola-1(1,3)- cyclopentanacyclododecaphan-7- en-11-one |
| 16 | 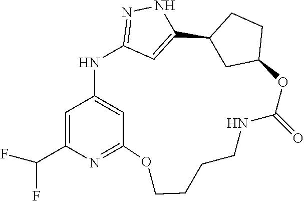 [SMILES: O=C1NCCCCOc2cc(cc(C(F)F)n2)Nc2cc([nH]n2)[C@H]2CC[C@H](C2)O1] | (1¹S,1³R,Z)-46-(difluoromethyl)-2¹H- 5,12-dioxa-3,10-diaza-4(4,2)- pyridina-2(5,3)-pyrazola-1(1,3)- cyclopentanacyclododecaphan-11- one |
| 17 | 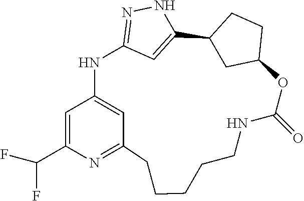 [SMILES: O=C1NCCCCCc2cc(cc(C(F)F)n2)Nc2cc([nH]n2)[C@H]2CC[C@H](C2)O1] | (1¹S,1³R,Z)-46-(difluoromethyl)-2¹H- 12-oxa-3,10-diaza-4(4,2)-pyridina- 2(3,5)-pyrazola-1(1,3)- cyclopentanacyclododecaphan-11- one |
| 18 | 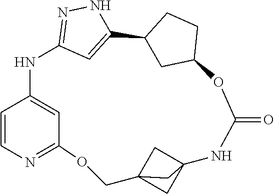 [SMILES: O=C1NC23CC(COc4cc(ccn4)Nc4cc([nH]n4)[C@H]4CC[C@H](C4)O1)(C2)C3] | (1¹S,1³R,Z)-2¹H-5,10-dioxa-3,8- diaza-4(4,2)-pyridina-2(3,5)- pyrazola-7(1,3)- bicyclo[1.1.1]pentana-1(1,3)- cyclopentanacyclodecaphan-9-one |
| 19 | 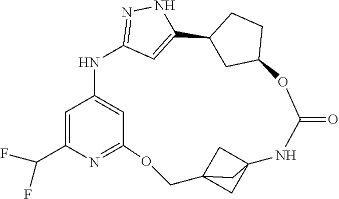 [SMILES: O=C1NC23CC(COc4cc(cc(C(F)F)n4)Nc4cc([nH]n4)[C@H]4CC[C@H](C4)O1)(C2)C3] | (1¹S,1³R,Z)-46-(difluoromethyl)-2¹H- 5,10-dioxa-3,8-diaza-4(4,2)- pyridina-2(3,5)-pyrazola-7(1,3)- bicyclo[1.1.1]pentana-1(1,3)- cyclopentanacyclodecaphan-9-one |
| 20 | 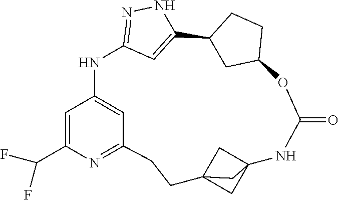 [SMILES: O=C1NC23CC(CCc4cc(cc(C(F)F)n4)Nc4cc([nH]n4)[C@H]4CC[C@H](C4)O1)(C2)C3] | (1¹S,1³R,Z)-46-(difluoromethyl)-2¹H- 10-oxa-3,8-diaza-4(4,2)-pyridina- 2(3,5)-pyrazola-7(1,3)- bicyclo[1.1.1]pentana-1(1,3)- cyclopentanacyclodecaphan-9-one |
| 21 | 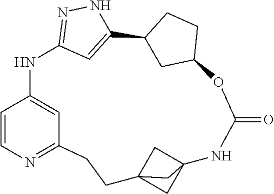 [SMILES: O=C1NC23CC(CCc4cc(ccn4)Nc4cc([nH]n4)[C@H]4CC[C@H](C4)O1)(C2)C3] | (1¹S,1³R,Z)-2¹H-10-oxa-3,8-diaza- 4(4,2)-pyridina-2(3,5)-pyrazola- 7(1,3)-bicyclo[1.1.1]pentana-1(1,3)- cyclopentanacyclodecaphan-9-one |
| 22 | 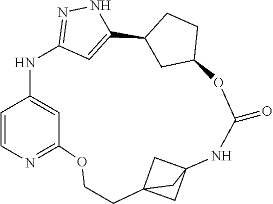 [SMILES: O=C1NC23CC(CCOc4cc(ccn4)Nc4cc([nH]n4)[C@H]4CC[C@H](C4)O1)(C2)C3] | (1¹S,1³R,Z)-2¹H-5,11-dioxa-3,9- diaza-4(4,2)-pyridina-2(3,5)- pyrazola-8(1,3)- bicyclo[1.1.1]pentana-1(1,3)- cyclopentanacycloundecaphan-10- one |
| 23 | 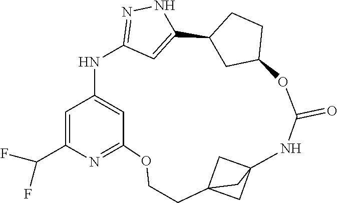 [SMILES: O=C1NC23CC(CCOc4cc(cc(C(F)F)n4)Nc4cc([nH]n4)[C@H]4CC[C@H](C4)O1)(C2)C3] | (1¹S,1³R,Z)-4⁶-(difluoromethyl)-21H- 5,11-dioxa-3,9-diaza-4(4,2)- pyridina-2(3,5)-pyrazola-8(1,3)- bicyclo[1.1.1]pentana-1(1,3)- cyclopentanacycloundecaphan-10- one |
| 24 | 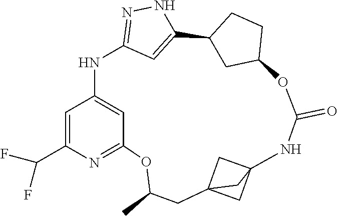 [SMILES: C[C@@H]1CC23CC(C2)(C3)NC(=O)O[C@@H]2CC[C@@H](C2)c2cc(n[nH]2)Nc2cc(nc(C(F)F)c2)O1] | (1¹S,1³R,6R,Z)-4⁶-(difluoromethyl)- 6-methyl-21H-5,11-dioxa-3,9-diaza- 4(4,2)-pyridina-2(3,5)-pyrazola- 8(1,3)-bicyclo[1.1.1]pentana-1(1,3)- cyclopentanacycloundecaphan-10- one |
| 25 | 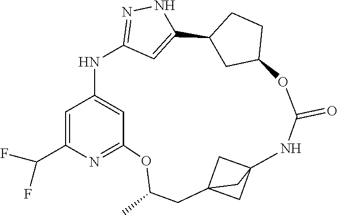 [SMILES: C[C@H]1CC23CC(C2)(C3)NC(=O)O[C@@H]2CC[C@@H](C2)c2cc(n[nH]2)Nc2cc(nc(C(F)F)c2)O1] | (1¹S,1³R,6S,Z)-4⁶-(difluoromethyl)- 6-methyl-21H-5,11-dioxa-3,9-diaza- 4(4,2)-pyridina-2(3,5)-pyrazola- 8(1,3)-bicyclo[1.1.1]pentana-1(1,3)- cyclopentanacycloundecaphan-10- one |
| 26 | 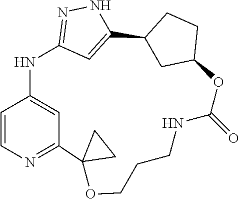 [SMILES: O=C1NCCCOC2(CC2)c2cc(ccn2)Nc2cc([nH]n2)[C@H]2CC[C@H](C2)O1] | (1′S,3′R,Z)-spiro[cyclopropane-1,5′- 6,12-dioxa-3,10-diaza-4(4,2)- pyridina-2(3,5)-pyrazola-1(1,3)- cyclopentanacyclododecaphan]-11′- one |
| 27 | 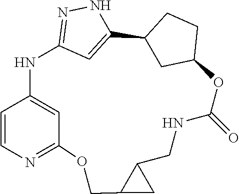 [SMILES: O=C1NCC2CC2COc2cc(ccn2)Nc2cc([nH]n2)[C@H]2CC[C@H](C2)O1] | (1¹S,1³R,Z)-2¹H-5,11-dioxa-3,9- diaza-4(4,2)-pyridina-2(3,5)- pyrazola-1(1,3)-cyclopentana- 7(1,2)- cyclopropanacycloundecaphan-10- one |
| 28 | 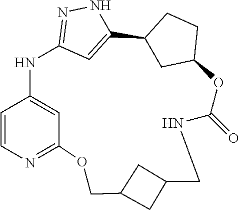 [SMILES: O=C1NCC2CC(COc3cc(ccn3)Nc3cc([nH]n3)[C@H]3CC[C@H](C3)O1)C2] | (1¹S,1³R,Z)-2¹H-5,11-dioxa-3,9- diaza-4(4,2)-pyridina-2(3,5)- pyrazola-1(1,3)-cyclopentana- 7(1,3)- cyclobutanacycloundecaphan-10- one |
| 29 | 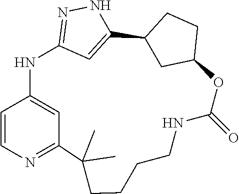 [SMILES: CC1(C)CCCCNC(=O)O[C@@H]2CC[C@@H](C2)c2cc(n[nH]2)Nc2ccnc1c2] | (1¹S,1³R,Z)-5,5-dimethyl-2¹H-12- oxa-3, 10-diaza-4(4,2)-pyridina- 2(3,5)-pyrazola-1(1,3)- cyclopentanacyclododecaphan-11- one |
| 30 | 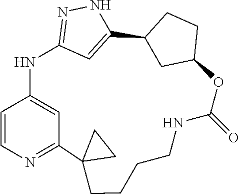 [SMILES: O=C1NCCCCC2(CC2)c2cc(ccn2)Nc2cc([nH]n2)[C@H]2CC[C@H](C2)O1] | (1′S,3′R,Z)-spiro[cyclopropane-1,5′- 12-oxa-3,10-diaza-4(4,2)-pyridina- 2(3,5)-pyrazola-1(1,3)- cyclopentanacyclododecaphan]-11′- one |
| 31 | 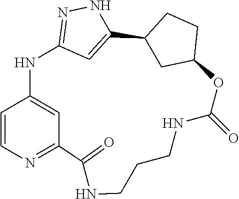 [SMILES: O=C1NCCCNC(=O)c2cc(ccn2)Nc2cc([nH]n2)[C@H]2CC[C@H](C2)O1] | (1¹S,1³R,Z)-2¹H-12-oxa-3,6,10- triaza-4(4,2)-pyridina-2(3,5)- pyrazola-1(1,3)- cyclopentanacyclododecaphane- 5,11-dione |
| 32 | 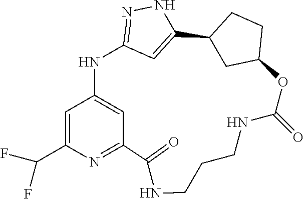 [SMILES: O=C1NCCCNC(=O)c2cc(cc(C(F)F)n2)Nc2cc([nH]n2)[C@H]2CC[C@H](C2)O1] | (1¹S,1³R,Z)-46-(difluoromethyl)-2¹H- 12-oxa-3,6,10-triaza-4(4,2)-pyridina- 2(3,5)-pyrazola-1(1,3)- cyclopentanacyclododecaphane- 5,11-dione |
| 33 | 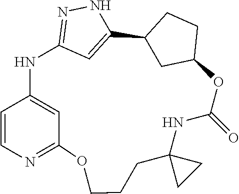 [SMILES: O=C1NC2(CCCOc3cc(ccn3)Nc3cc([nH]n3)[C@H]3CC[C@H](C3)O1)CC2] | (1′S,3′R,Z)-spiro[cyclopropane-1,9′- 5,12-dioxa-3,10-diaza-4(4,2)- pyridina-2(3,5)-pyrazola-1(1,3)- cyclopentanacyclododecaphan]-11′- one |
| 34 | 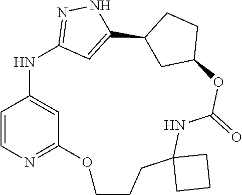 [SMILES: O=C1NC2(CCCOc3cc(ccn3)Nc3cc([nH]n3)[C@H]3CC[C@H](C3)O1)CCC2] | (1′S,3′R,Z)-spiro[cyclobutane-1,9′- 5,12-dioxa-3,10-diaza-4(4,2)- pyridina-2(3,5)-pyrazola-1(1,3)- cyclopentanacyclododecaphan]-11′- one |
| 35 | 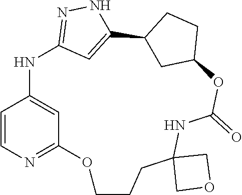 [SMILES: O=C1NC2(CCCOc3cc(ccn3)Nc3cc([nH]n3)[C@H]3CC[C@H](C3)O1)COC2] | (1′S,3′R,Z)-spiro[oxetane-3,9′-5,12- dioxa-3,10-diaza-4(4,2)-pyridina- 2(3,5)-pyrazola-1(1,3)- cyclopentanacyclododecaphan]-11′- one |
| 36 | 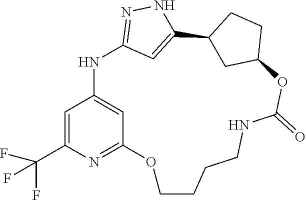 [SMILES: O=C1NCCCCOc2cc(cc(C(F)(F)F)n2)Nc2cc([nH]n2)[C@H]2CC[C@H](C2)O1] | (1¹S,1³R,Z)-46-(trifluoromethyl)-2¹H- 5,12-dioxa-3,10-diaza-4(4,2)- pyridina-2(3,5)-pyrazola-1(1,3)- cyclopentanacyclododecaphan-11- one |
| 37 | 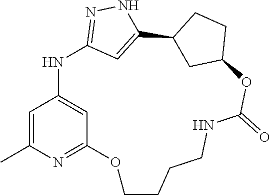 [SMILES: Cc1cc2cc(n1)OCCCCNC(=O)O[C@@H]1CC[C@@H](C1)c1cc(n[nH]1)N2] | (1¹S,1³R,Z)-46-methyl-2¹H-5,12- dioxa-3,10-diaza-4(4,2)-pyridina- 2(3,5)-pyrazola-1(1,3)- cyclopentanacyclododecaphan-11- one |
| 38 | 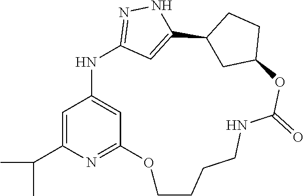 [SMILES: CC(C)c1cc2cc(n1)OCCCCNC(=O)O[C@@H]1CC[C@@H](C1)c1cc(n[nH]1)N2] | (1¹S,1³R,Z)-46-isopropyl-2¹H-5,12- dioxa-3,10-diaza-4(4,2)-pyridina- 2(3,5)-pyrazola-1(1,3)- cyclopentanacyclododecaphan-11- one |
| 39 | 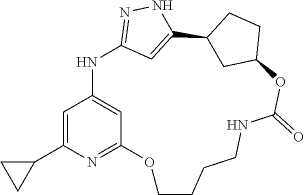 [SMILES: O=C1NCCCCOc2cc(cc(C3CC3)n2)Nc2cc([nH]n2)[C@H]2CC[C@H](C2)O1] | (1¹S,1³R,Z)-46-cyclopropyl-2¹H- 5,12-dioxa-3,10-diaza-4(4,2)- pyridina-2(3,5)-pyrazola-1(1,3)- cyclopentanacyclododecaphan-11- one |
| 40 | 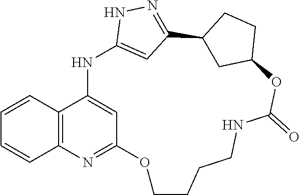 [SMILES: O=C1NCCCCOc2cc(c3ccccc3n2)Nc2cc(n[nH]2)[C@H]2CC[C@H](C2)O1] | (1¹S,1³R,Z)-2¹H-5,12-dioxa-3,10- diaza-4(4,2)-quinolina-2(3,5)- pyrazola-1(1,3)- cyclopentanacyclododecaphan-11- one |
| 41 | 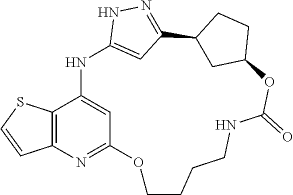 [SMILES: O=C1NCCCCOc2cc(c3sccc3n2)Nc2cc(n[nH]2)[C@H]2CC[C@H](C2)O1] | (1¹S,1³R,Z)-2¹H-5,12-dioxa-3,10- diaza-4(7,5)-thieno[3,2-b]pyridina- 2(3,5)-pyrazola-1(1,3)- cyclopentanacyclododecaphan-11- one |
| 42 | 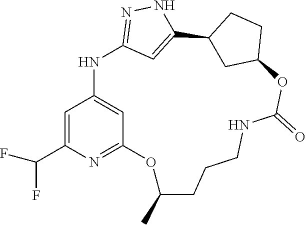 [SMILES: C[C@@H]1CCCNC(=O)O[C@@H]2CC[C@@H](C2)c2cc(n[nH]2)Nc2cc(nc(C(F)F)c2)O1] | (1¹S,1³R,6R,Z)-46-(difluoromethyl)- 6-methyl-2¹H-5,12-dioxa-3,10- diaza-4(4,2)-pyridina-2(5,3)- pyrazola-1(1,3)- cyclopentanacyclododecaphan-11- one |
| 43 | 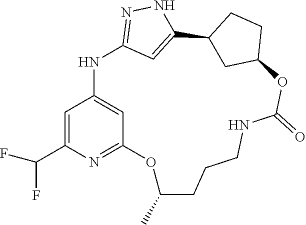 [SMILES: C[C@H]1CCCNC(=O)O[C@@H]2CC[C@@H](C2)c2cc(n[nH]2)Nc2cc(nc(C(F)F)c2)O1] | (1¹S,1³R,6S,Z)-46-(difluoromethyl)- 6-methyl-2¹H-5,12-dioxa-3,10- diaza-4(4,2)-pyridina-2(5,3)- pyrazola-1(1,3)- cyclopentanacyclododecaphan-11- one |
| 44 | 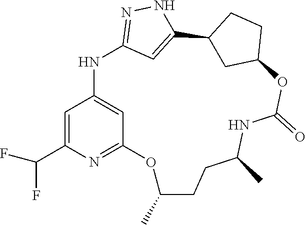 [SMILES: C[C@H]1CC[C@H](C)Oc2cc(cc(C(F)F)n2)Nc2cc([nH]n2)[C@H]2CC[C@H](C2)OC(=O)N1] | (1¹S,1³R,6S,9S,Z)-46- (difluoromethyl)-6,9-dimethyl-2¹H- 5,12-dioxa-3,10-diaza-4(4,2)- pyridina-2(5,3)-pyrazola-1(1,3)- cyclopentanacyclododecaphan-11- one |
| 45 | 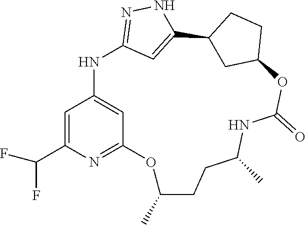 [SMILES: C[C@@H]1CC[C@H](C)Oc2cc(cc(C(F)F)n2)Nc2cc([nH]n2)[C@H]2CC[C@H](C2)OC(=O)N1] | (1¹S,1³R,6S,9R,Z)-46- (difluoromethyl)-6,9-dimethyl-2¹H- 5,12-dioxa-3,10-diaza-4(4,2)- pyridina-2(5,3)-pyrazola-1(1,3)- cyclopentanacyclododecaphan-11- one |
| 46 | 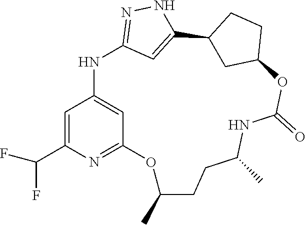 [SMILES: C[C@@H]1CC[C@@H](C)Oc2cc(cc(C(F)F)n2)Nc2cc([nH]n2)[C@H]2CC[C@H](C2)OC(=O)N1] | (1¹S,1³R,6R,9R,Z)-46- (difluoromethyl)-6,9-dimethyl-2¹H- 5,12-dioxa-3,10-diaza-4(4,2)- pyridina-2(5,3)-pyrazola-1(1,3)- cyclopentanacyclododecaphan-11- one |
| 47 | 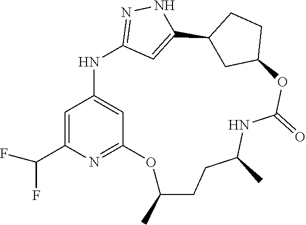 [SMILES: C[C@@H]1CC[C@H](C)NC(=O)O[C@@H]2CC[C@@H](C2)c2cc(n[nH]2)Nc2cc(nc(C(F)F)c2)O1] | (1¹S,1³R,6R,9S,Z)-4⁶- (difluoromethyl)-6,9-dimethyl-2¹H- 5,12-dioxa-3,10-diaza-4(4,2)- pyridina-2(5,3)-pyrazola-1(1,3)- cyclopentanacyclododecaphan-11- one |
| 48 | 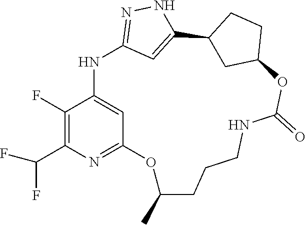 [SMILES: C[C@@H]1CCCNC(=O)O[C@@H]2CC[C@@H](C2)c2cc(n[nH]2)Nc2cc(nc(C(F)F)c2F)O1] | (1¹S,1³R,6R,Z)-4⁶-(difluoromethyl)- 45-fluoro-6-methyl-2¹H-5,12-dioxa- 3,10-diaza-4(4,2)-pyridina-2(5,3)- pyrazola-1(1,3)- cyclopentanacyclododecaphan-11- one |
| 49 | 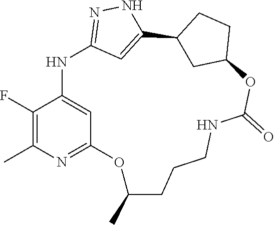 [SMILES: Cc1nc2cc(c1F)Nc1cc([nH]n1)[C@H]1CC[C@H](C1)OC(=O)NCCC[C@@H](C)O2] | (1¹S,1³R,6R,Z)-4⁵-fluoro-4⁶,6- dimethyl-2¹H-5,12-dioxa-3,10-diaza- 4(4,2)-pyridina-2(5,3)-pyrazola- 1(1,3)- cyclopentanacyclododecaphan-11- one |
| 50 | 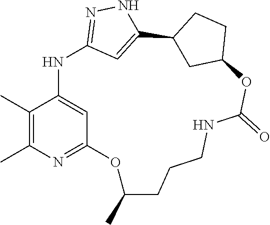 [SMILES: Cc1nc2cc(c1C)Nc1cc([nH]n1)[C@H]1CC[C@H](C1)OC(=O)NCCC[C@@H](C)O2] | (1¹S,1³R,6R,Z)-4⁵,4⁶,6-trimethyl- 2¹H-5,12-dioxa-3,10-diaza-4(4,2)- pyridina-2(5,3)-pyrazola-1(1,3)- cyclopentanacyclododecaphan-11- one |
| 51 | 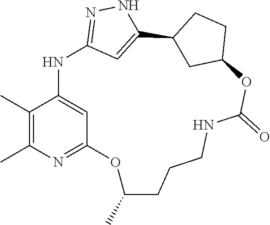 [SMILES: Cc1nc2cc(c1C)Nc1cc([nH]n1)[C@H]1CC[C@H](C1)OC(=O)NCCC[C@H](C)O2] | (1¹S,1³R,6S,Z)-4⁵,4⁶,6-trimethyl- 21H-5,12-dioxa-3,10-diaza-4(4,2)- pyridina-2(5,3)-pyrazola-1(1,3)- cyclopentanacyclododecaphan-11- one |
| 52 | 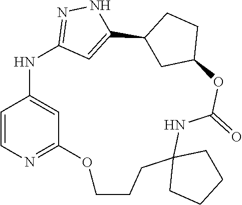 [SMILES: O=C1NC2(CCCC2)CCCOc2cc(ccn2)Nc2cc([nH]n2)[C@H]2CC[C@H](C2)O1] | (1′S,3′R,Z)-spiro[cyclopentane-1,9′- 5,12-dioxa-3,10-diaza-4(4,2)- pyridina-2(3,5)-pyrazola-1(1,3)- cyclopentanacyclododecaphan]-11′- one |
| 53 | 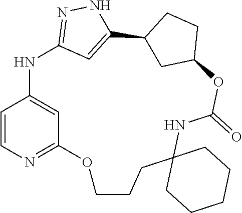 [SMILES: O=C1NC2(CCCCC2)CCCOc2cc(ccn2)Nc2cc([nH]n2)[C@H]2CC[C@H](C2)O1] | (1′S,3′R,Z)-spiro[cyclohexane-1,9′- 5,12-dioxa-3,10-diaza-4(4,2)- pyridina-2(3,5)-pyrazola-1(1,3)- cyclopentanacyclododecaphan]-11′- one |
| 54 | 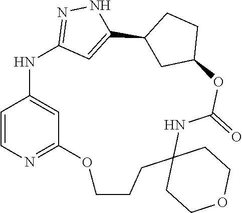 [SMILES: O=C1NC2(CCCOc3cc(ccn3)Nc3cc([nH]n3)[C@H]3CC[C@H](C3)O1)CCOCC2] | (1′S,3′R,Z)-2,3,5,6- tetrahydrospiro[pyran-4,9′-5,12- dioxa-3,10-diaza-4(4,2)-pyridina- 2(3,5)-pyrazola-1(1,3)- cyclopentanacyclododecaphan]-11′- one |
| 55 | 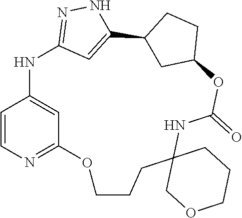 [SMILES: O=C1NC2(CCCOC2)CCCOc2cc(ccn2)Nc2cc([nH]n2)[C@H]2CC[C@H](C2)O1] | (1′S,3′R,Z)-5,6-dihydro-2H,4H- spiro[pyran-3,9′-5,12-dioxa-3,10- diaza-4(4,2)-pyridina-2(3,5)- pyrazola-1(1,3)- cyclopentanacyclododecaphan]-11′- one |
| 56 | 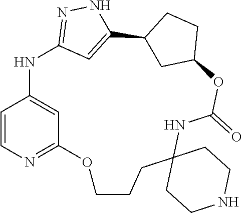 [SMILES: O=C1NC2(CCCOc3cc(ccn3)Nc3cc([nH]n3)[C@H]3CC[C@H](C3)O1)CCNCC2] | (1′S,3′R,Z)-spiro[piperidine-4,9′- 5,12-dioxa-3,10-diaza-4(4,2)- pyridina-2(3,5)-pyrazola-1(1,3)- cyclopentanacyclododecaphan]-11′- one |
| 57 | 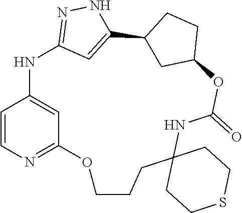 [SMILES: O=C1NC2(CCCOc3cc(ccn3)Nc3cc([nH]n3)[C@H]3CC[C@H](C3)O1)CCSCC2] | (1′S,3′R,Z)-2,3,5,6- tetrahydrospiro[thiopyran-4,9′-5,12- dioxa-3,10-diaza-4(4,2)-pyridina- 2(3,5)-pyrazola-1(1,3)- cyclopentanacyclododecaphan]-11′- one |
| 58 | 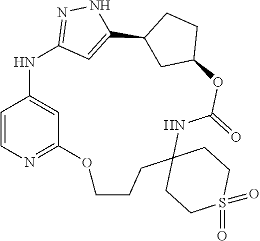 [SMILES: O=C1NC2(CCCOc3cc(ccn3)Nc3cc([nH]n3)[C@H]3CC[C@H](C3)O1)CCS(=O)(=O)CC2] | (1′S,3′R,Z)-2,3,5,6- tetrahydrospiro[thiopyran-4,9′-5,12- dioxa-3,10-diaza-4(4,2)-pyridina- 2(3,5)-pyrazola-1(1,3)- cyclopentanacyclododecaphan]-11′- one 1,1-dioxide |
| 59 | 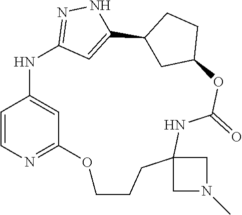 [SMILES: CN1CC2(CCCOc3cc(ccn3)Nc3cc([nH]n3)[C@H]3CC[C@H](C3)OC(=O)N2)C1] | (1′S,3′R,Z)-1-methylspiro[azetidine- 3,9′-5,12-dioxa-3,10-diaza-4(4,2)- pyridina-2(3,5)-pyrazola-1(1,3)- cyclopentanacyclododecaphan]-11′- one |
| 60 | 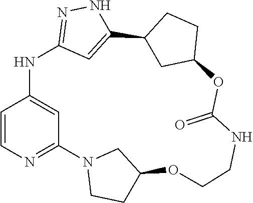 [SMILES: O=C1NCCO[C@H]2CCN(C2)c2cc(ccn2)Nc2cc([nH]n2)[C@H]2CC[C@H](C2)O1] | (1³S,5¹S,5³R,Z)-4¹H-6,11-dioxa-3,8- diaza-2(2,4)-pyridina-4(5,3)- pyrazola-1(1,3)-pyrrolidina-5(1,3)- cyclopentanacycloundecaphan-7- one |
| 61 | 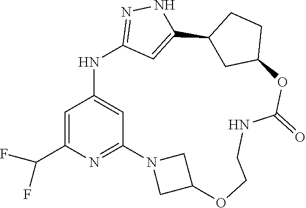 [SMILES: O=C1NCCOC2CN(C2)c2cc(cc(C(F)F)n2)Nc2cc([nH]n2)[C@H]2CC[C@H](C2)O1] | (5¹S,5³R,Z)-2⁶-(difluoromethyl)-4¹H- 6,11-dioxa-3,8-diaza-2(2,4)- pyridina-4(5,3)-pyrazola-1(1,3)- azetidina-5(1,3)- cyclopentanacycloundecaphan-7- one |
| 62 | 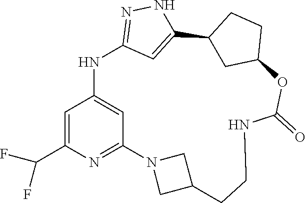 [SMILES: O=C1NCCC2CN(C2)c2cc(cc(C(F)F)n2)Nc2cc([nH]n2)[C@H]2CC[C@H](C2)O1] | (5¹S,5³R,Z)-2⁶-(difluoromethyl)-4¹H- 6-oxa-3,8-diaza-2(2,4)-pyridina- 4(5,3)-pyrazola-1(1,3)-azetidina- 5(1,3)-cyclopentanacyclodecaphan- 7-one |
| 63 | 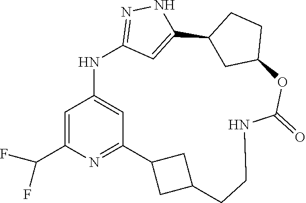 [SMILES: O=C1NCCC2CC(C2)c2cc(cc(C(F)F)n2)Nc2cc([nH]n2)[C@H]2CC[C@H](C2)O1] | (5¹S,5³R,Z)-2⁶-(difluoromethyl)-4¹H- 6-oxa-3,8-diaza-2(2,4)-pyridina- 4(5,3)-pyrazola-5(1,3)- cyclopentana-1(1,3)- cyclobutanacyclodecaphan-7-one |
| 64 | 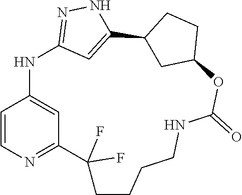 [SMILES: O=C1NCCCCC(F)(F)c2cc(ccn2)Nc2cc([nH]n2)[C@H]2CC[C@H](C2)O1] | (1¹S,1³R,Z)-5,5-difluoro-2¹H-12- oxa-3,10-diaza-4(4,2)-pyridina- 2(3,5)-pyrazola-1(1,3)- cyclopentanacyclododecaphan-11- one |
| 65 | 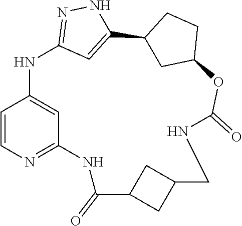 [SMILES: O=C1NCC2CC(C2)C(=O)Nc2cc(ccn2)Nc2cc([nH]n2)[C@H]2CC[C@H](C2)O1] | (1¹S,1³R,Z)-2¹H-11-oxa-3,5,9- triaza-4(4,2)-pyridina-2(3,5)- pyrazola-1(1,3)-cyclopentana- 7(1,3)- cyclobutanacycloundecaphane- 6,10-dione |
| 66 | 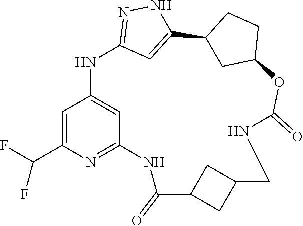 [SMILES: O=C1NCC2CC(C2)C(=O)Nc2cc(cc(C(F)F)n2)Nc2cc([nH]n2)[C@H]2CC[C@H](C2)O1] | (1¹S,1³R,Z)-46-(difluoromethyl)-2¹H- 11-oxa-3,5,9-triaza-4(4,2)-pyridina- 2(3,5)-pyrazola-1(1,3)- cyclopentana-7(1,3)- cyclobutanacycloundecaphane- 6,10-dione |
| 67 | 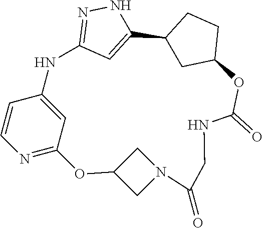 [SMILES: O=C1NCC(=O)N2CC(C2)Oc2cc(ccn2)Nc2cc([nH]n2)[C@H]2CC[C@H](C2)O1] | (1¹S,1³R,Z)-2¹H-5,11-dioxa-3,9- diaza-4(4,2)-pyridina-2(3,5)- pyrazola-6(3,1)-azetidina-1(1,3)- cyclopentanacycloundecaphane- 7,10-dione |
| 68 | 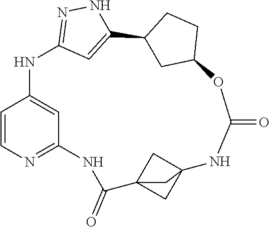 [SMILES: O=C1NC23CC(C2)(C3)C(=O)Nc2cc(ccn2)Nc2cc([nH]n2)[C@H]2CC[C@H](C2)O1] | (1¹S,1³R,Z)-2¹H-10-oxa-3,5,8- triaza-4(4,2)-pyridina-2(3,5)- pyrazola-7(1,3)- bicyclo[1.1.1]pentana-1(1,3)- cyclopentanacyclodecaphane-6,9- dione |
| 69 | 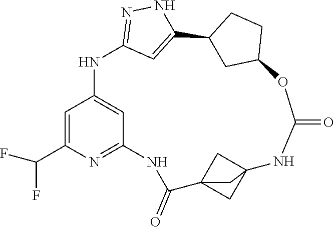 [SMILES: O=C1NC23CC(C2)(C3)C(=O)Nc2cc(cc(C(F)F)n2)Nc2cc([nH]n2)[C@H]2CC[C@H](C2)O1] | (1¹S,1³R,Z)-4⁶-(difluoromethyl)-2¹H- 10-oxa-3,5,8-triaza-4(4,2)-pyridina- 2(3,5)-pyrazola-7(1,3)- bicyclo[1.1.1]pentana-1(1,3)- cyclopentanacyclodecaphane-6,9- dione |
| 70 | 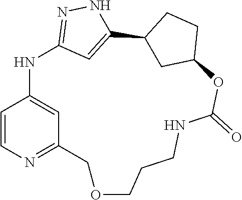 [SMILES: O=C1NCCCOCc2cc(ccn2)Nc2cc([nH]n2)[C@H]2CC[C@H](C2)O1] | (1¹S,1³R,Z)-2¹H-6,12-dioxa-3,10- diaza-4(4,2)-pyridina-2(3,5)- pyrazola-1(1,3)- cyclopentanacyclododecaphan-11- one |
| 71 | 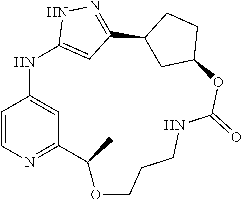 [SMILES: C[C@H]1OCCCNC(=O)O[C@@H]2CC[C@@H](C2)c2cc([nH]n2)Nc2ccnc1c2] | (1¹S,1³R,5R,Z)-5-methyl-2¹H-6,12- dioxa-3,10-diaza-4(4,2)-pyridina- 2(3,5)-pyrazola-1(1,3)- cyclopentanacyclododecaphan-11- one |
| 72 | 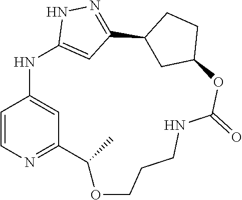 [SMILES: C[C@@H]1OCCCNC(=O)O[C@@H]2CC[C@@H](C2)c2cc([nH]n2)Nc2ccnc1c2] | (1¹S,1³R,5S,Z)-5-methyl-2¹H-6,12- dioxa-3,10-diaza-4(4,2)-pyridina- 2(3,5)-pyrazola-1(1,3)- cyclopentanacyclododecaphan-11- one |
| 73 | 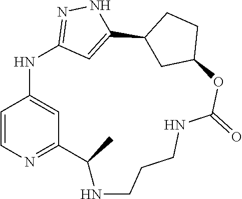 [SMILES: C[C@H]1NCCCNC(=O)O[C@@H]2CC[C@@H](C2)c2cc(n[nH]2)Nc2ccnc1c2] | (1¹S,1³R,5R,Z)-5-methyl-2¹H-12- oxa-3,6,10-triaza-4(4,2)-pyridina- 2(3,5)-pyrazola-1(1,3)- cyclopentanacyclododecaphan-11- one |
| 74 | 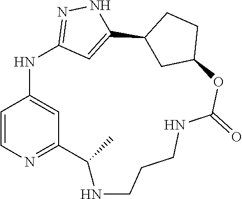 [SMILES: C[C@@H]1NCCCNC(=O)O[C@@H]2CC[C@@H](C2)c2cc(n[nH]2)Nc2ccnc1c2] | (1¹S,1³R,5S,Z)-5-methyl-2¹H-12- oxa-3,6,10-triaza-4(4,2)-pyridina- 2(3,5)-pyrazola-1(1,3)- cyclopentanacyclododecaphan-11- one |
| 75 | 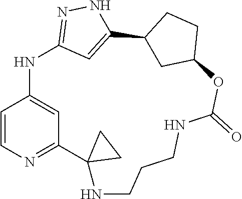 [SMILES: O=C1NCCCNC2(CC2)c2cc(ccn2)Nc2cc([nH]n2)[C@H]2CC[C@H](C2)O1] | (1′S,3′R,Z)-spiro[cyclopropane-1,5′- 12-oxa-3,6,10-triaza-4(4,2)-pyridina- 2(3,5)-pyrazola-1(1,3)- cyclopentanacyclododecaphan]-11′- one |
| 76 | 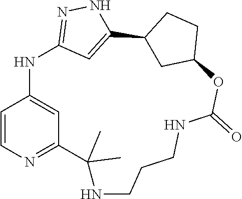 [SMILES: CC1(C)NCCCNC(=O)O[C@@H]2CC[C@@H](C2)c2cc(n[nH]2)Nc2ccnc1c2] | (1¹S,1³R,Z)-5,5-dimethyl-2¹H-12- oxa-3,6,10-triaza-4(4,2)-pyridina- 2(3,5)-pyrazola-1(1,3)- cyclopentanacyclododecaphan-11- one |
| 77 | 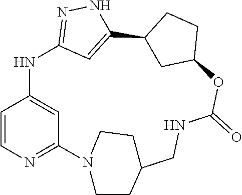 [SMILES: O=C1NCC2CCN(CC2)c2cc(ccn2)Nc2cc([nH]n2)[C@H]2CC[C@H](C2)O1] | (5¹S,5³R,Z)-4¹H-6-oxa-3,8-diaza- 2(2,4)-pyridina-1(1,4)-piperidina- 4(5,3)-pyrazola-5(1,3)- cyclopentanacyclononaphan-7-one |
| 78 | 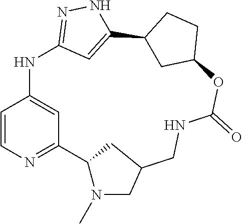 [SMILES: CN1CC2CNC(=O)O[C@@H]3CC[C@@H](C3)c3cc(n[nH]3)Nc3ccnc(c3)[C@@H]1C2] | (1²S,5¹S,5³R,Z)-11-methyl-4¹H-6- oxa-3,8-diaza-2(2,4)-pyridina- 4(5,3)-pyrazola-1(2,4)-pyrrolidina- 5(1,3)-cyclopentanacyclononaphan- 7-one |
| 79 | 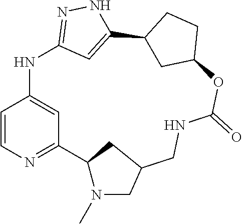 [SMILES: CN1CC2CNC(=O)O[C@@H]3CC[C@@H](C3)c3cc(n[nH]3)Nc3ccnc(c3)[C@H]1C2] | (1²R,5¹S,5³R,Z)-11-methyl-4¹H-6- oxa-3,8-diaza-2(2,4)-pyridina- 4(5,3)-pyrazola-1(2,4)-pyrrolidina- 5(1,3)-cyclopentanacyclononaphan- 7-one |
| 80 | 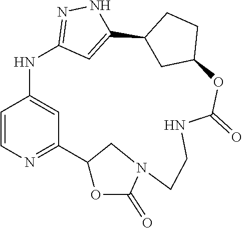 [SMILES: O=C1NCCN2CC(OC2=O)c2cc(ccn2)Nc2cc([nH]n2)[C@H]2CC[C@H](C2)O1] | (1²R,5¹S,5³R,Z)-11-methyl-4¹H-6- oxa-3,8-diaza-2(2,4)-pyridina- 4(5,3)-pyrazola-1(2,4)-pyrrolidina- 5(1,3)-cyclopentanacyclononaphan- 7-one |
| 81 | 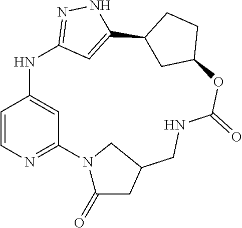 [SMILES: O=C1NCC2CC(=O)N(C2)c2cc(ccn2)Nc2cc([nH]n2)[C@H]2CC[C@H](C2)O1] | (5¹S,5³R,Z)-4¹H-6-oxa-3,8-diaza- 2(2,4)-pyridina-4(5,3)-pyrazola- 1(1,3)-pyrrolidina-5(1,3)- cyclopentanacyclononaphane-15,7- dione |
| 82 | 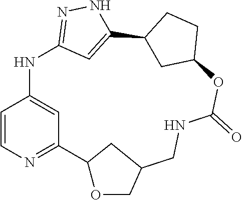 [SMILES: O=C1NCC2COC(C2)c2cc(ccn2)Nc2cc([nH]n2)[C@H]2CC[C@H](C2)O1] | (5¹S,5³R,Z)-12,13,14,15-tetrahydro- 4¹H-6-oxa-3,8-diaza-2(2,4)-pyridina- 4(5,3)-pyrazola-1(2,4)-furana- 5(1,3)-cyclopentanacyclononaphan- 7-one |
| 83 | 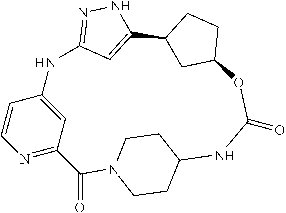 [SMILES: O=C1NC2CCN(CC2)C(=O)c2cc(ccn2)Nc2cc([nH]n2)[C@H]2CC[C@H](C2)O1] | (1¹S,1³R,Z)-2¹H-9-oxa-3,7-diaza- 4(4,2)-pyridina-6(1,4)-piperidina- 2(3,5)-pyrazola-1(1,3)- cyclopentanacyclononaphane-5,8- dione |
| 84 | 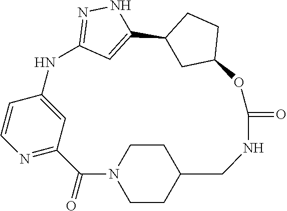 [SMILES: O=C1NCC2CCN(CC2)C(=O)c2cc(ccn2)Nc2cc([nH]n2)[C@H]2CC[C@H](C2)O1] | (1¹S,1³R,Z)-2¹H-10-oxa-3,8-diaza- 4(4,2)-pyridina-6(1,4)-piperidina- 2(3,5)-pyrazola-1(1,3)- cyclopentanacyclodecaphane-5,9- dione |
| 85 | 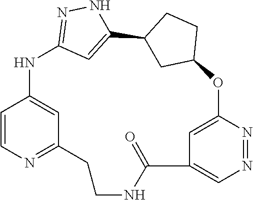 [SMILES: O=C1NCCc2cc(ccn2)Nc2cc([nH]n2)[C@H]2CC[C@H](C2)Oc2cc1cnn2] | (2¹S,2³R,Z)-1¹H-3-oxa-6,10-diaza- 4(3,5)-pyridazina-9(2,4)-pyridina- 1(3,5)-pyrazola-2(1,3)- cyclopentanacyclodecaphan-5-one |
| 86 | 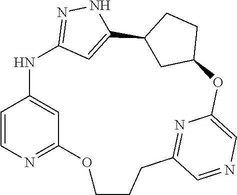 [SMILES: c1cc2cc(n1)OCCCc1cncc(n1)O[C@@H]1CC[C@@H](C1)c1cc(n[nH]1)N2] | (2¹S,2³R,Z)-1¹H-3,8-dioxa-10-aza- 4(2,6)-pyrazina-9(2,4)-pyridina- 1(3,5)-pyrazola-2(1,3)- cyclopentanacyclodecaphane |
| 87 | 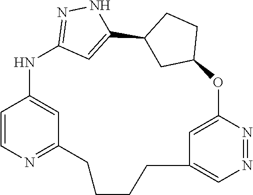 [SMILES: c1cc2cc(n1)CCCCc1cnnc(c1)O[C@@H]1CC[C@@H](C1)c1cc(n[nH]1)N2] | (2¹S,2³R,Z)-1¹H-3-oxa-10-aza- 4(3,5)-pyridazina-9(2,4)-pyridina- 1(3,5)-pyrazola-2(1,3)- cyclopentanacyclodecaphane |
| 88 | 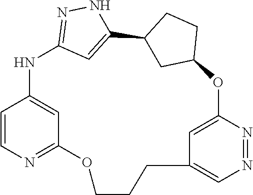 [SMILES: c1cc2cc(n1)OCCCc1cnnc(c1)O[C@@H]1CC[C@@H](C1)c1cc(n[nH]1)N2] | (2¹S,2³R,Z)-1¹H-3,8-dioxa-10-aza- 4(3,5)-pyridazina-9(2,4)-pyridina- 1(3,5)-pyrazola-2(1,3)- cyclopentanacyclodecaphane |
| 89 | 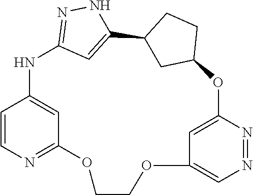 [SMILES: c1cc2cc(n1)OCCOc1cnnc(c1)O[C@@H]1CC[C@@H](C1)c1cc(n[nH]1)N2] | (2¹S,2³R,Z)-1¹H-3,5,8-trioxa-10- aza-4(3,5)-pyridazina-9(2,4)- pyridina-1(3,5)-pyrazola-2(1,3)- cyclopentanacyclodecaphane |
| 90 | 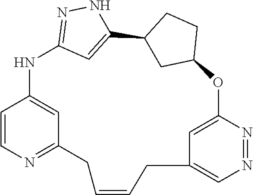 [SMILES: C1=C\Cc2cc(ccn2)Nc2cc([nH]n2)[C@H]2CC[C@H](C2)Oc2cc(cnn2)C/1] | (1⁴7,2¹S,2³R,6Z)-1¹H-3-oxa-10-aza- 4(3,5)-pyridazina-9(2,4)-pyridina- 1(3,5)-pyrazola-2(1,3)- cyclopentanacyclodecaphan-6-ene |
| 91 | 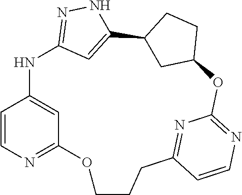 [SMILES: c1cc2cc(n1)OCCCc1ccnc(n1)O[C@@H]1CC[C@@H](C1)c1cc(n[nH]1)N2] | (2¹S,2³R,Z)-1¹H-3,8-dioxa-10-aza- 4(2,4)-pyrimidina-9(2,4)-pyridina- 1(3,5)-pyrazola-2(1,3)- cyclopentanacyclodecaphane |
| 92 | 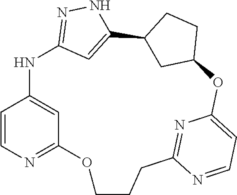 [SMILES: c1cc2cc(n1)OCCCc1nccc(n1)O[C@@H]1CC[C@@H](C1)c1cc(n[nH]1)N2] | (2¹S,2³R,Z)-1¹H-3,8-dioxa-10-aza- 4(4,2)-pyrimidina-9(2,4)-pyridina- 1(3,5)-pyrazola-2(1,3)- cyclopentanacyclodecaphane |
| 93 | 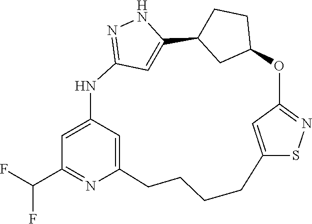 [SMILES: FC(F)c1cc2cc(n1)CCCCc1cc(ns1)O[C@@H]1CC[C@@H](C1)c1cc(n[nH]1)N2] | (1⁴Z,2¹S,2³R,4⁴Z)-9⁶- (difluoromethyl)-1¹H-3-oxa-10-aza- 4(3,5)-isothiazola-9(2,4)-pyridina- 1(3,5)-pyrazola-2(1,3)- cyclopentanacyclodecaphane |
| 94 | 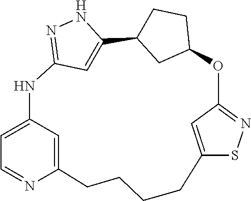 [SMILES: c1cc2cc(n1)CCCCc1cc(ns1)O[C@@H]1CC[C@@H](C1)c1cc(n[nH]1)N2] | (1⁴7,2¹S,2³R,4⁴Z)-1¹H-3-oxa-10- aza-4(3,5)-isothiazola-9(2,4)- pyridina-1(3,5)-pyrazola-2(1,3)- cyclopentanacyclodecaphane |
| 95 | 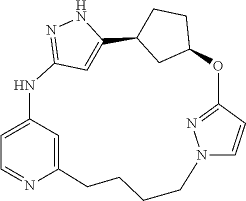 [SMILES: c1cc2cc(n1)CCCCn1ccc(n1)O[C@@H]1CC[C@@H](C1)c1cc(n[nH]1)N2] | (1¹S,1³R,2⁴Z,9²E)-2¹H,9¹H-10-oxa- 3-aza-4(4,2)-pyridina-2(3,5),9(1,3)- dipyrazola-1(1,3)- cyclopentanacyclodecaphane |
| 96 | 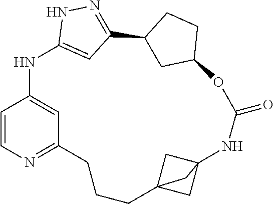 [SMILES: O=C1NC23CC(CCCc4cc(ccn4)Nc4cc(n[nH]4)[C@H]4CC[C@H](C4)O1)(C2)C3] | (1¹S,1³R,Z)-2¹H-11-oxa-3,9-diaza- 4(4,2)-pyridina-2(3,5)-pyrazola- 8(1,3)-bicyclo[1.1.1]pentana-1(1,3)- cyclopentanacycloundecaphan-10- one |
| 97 | 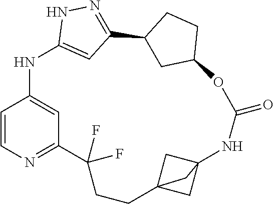 [SMILES: O=C1NC23CC(CCC(F)(F)c4cc(ccn4)Nc4cc(n[nH]4)[C@H]4CC[C@H](C4)O1)(C2)C3] | (1¹S,1³R,Z)-5,5-difluoro-2¹H-11- oxa-3,9-diaza-4(4,2)-pyridina- 2(3,5)-pyrazola-8(1,3)- bicyclo[1.1.1]pentana-1(1,3)- cyclopentanacycloundecaphan-10- one |
| 98 | 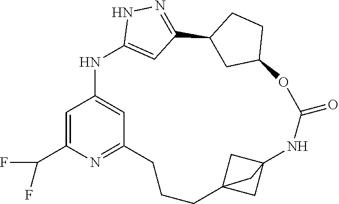 [SMILES: O=C1NC23CC(CCCc4cc(cc(C(F)F)n4)Nc4cc(n[nH]4)[C@H]4CC[C@H](C4)O1)(C2)C3] | (1¹S,1³R,Z)-4⁶-(difluoromethyl)-2¹H- 11-oxa-3,9-diaza-4(4,2)-pyridina- 2(3,5)-pyrazola-8(1,3)- bicyclo[1.1.1]pentana-1(1,3)- cyclopentanacycloundecaphan-10- one |
| 99 | 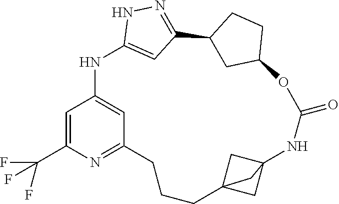 [SMILES: O=C1NC23CC(CCCc4cc(cc(C(F)(F)F)n4)Nc4cc(n[nH]4)[C@H]4CC[C@H](C4)O1)(C2)C3] | (1¹S,1³R,Z)-4⁶-(trifluoromethyl)-2¹H- 11-oxa-3,9-diaza-4(4,2)-pyridina- 2(3,5)-pyrazola-8(1,3)- bicyclo[1.1.1]pentana-1(1,3)- cyclopentanacycloundecaphan-10- one |
| 100 | 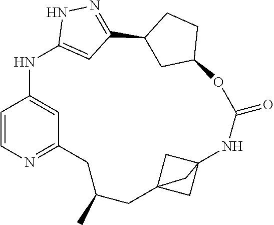 [SMILES: C[C@H]1Cc2cc(ccn2)Nc2cc(n[nH]2)[C@H]2CC[C@H](C2)OC(=O)NC23CC(C1)(C2)C3] | (1¹S,1³R,6R,Z)-6-methyl-2¹H-5,11- dioxa-3,9-diaza-4(4,2)-pyridina- 2(3,5)-pyrazola-8(1,3)- bicyclo[1.1.1]pentana-1(1,3)- cyclopentanacycloundecaphan-10- one |
| 101 | 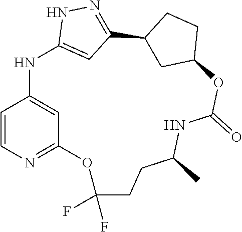 [SMILES: C[C@H]1CCC(F)(F)Oc2cc(ccn2)Nc2cc(n[nH]2)[C@H]2CC[C@H](C2)OC(=O)N1] | (1¹S,1³R,9S,Z)-6,6-difluoro-9- methyl-2¹H-5,12-dioxa-3,10-diaza- 4(4,2)-pyridina-2(3,5)-pyrazola- 1(1,3)- cyclopentanacyclododecaphan-11- one |
| 102 | 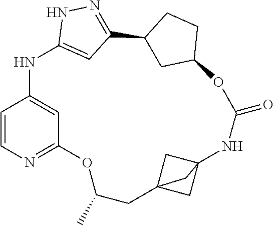 [SMILES: C[C@H]1CC23CC(C2)(C3)NC(=O)O[C@@H]2CC[C@@H](C2)c2cc([nH]n2)Nc2ccnc(c2)O1] | (1¹S,1³R,6S,Z)-6-methyl-2¹H-5,11- dioxa-3,9-diaza-4(4,2)-pyridina- 2(3,5)-pyrazola-8(1,3)- bicyclo[1.1.1]pentana-1(1,3)- cyclopentanacycloundecaphan-10- one |
| 103 | 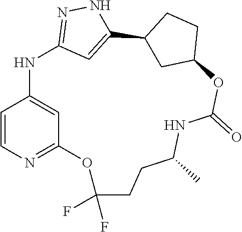 [SMILES: C[C@@H]1CCC(F)(F)Oc2cc(ccn2)Nc2cc([nH]n2)[C@H]2CC[C@H](C2)OC(=O)N1] | (1¹S,1³R,9R,Z)-6,6-difluoro-9- methyl-2¹H-5,12-dioxa-3,10-diaza- 4(4,2)-pyridina-2(3,5)-pyrazola- 1(1,3)- cyclopentanacyclododecaphan-11- one |
| 104 | 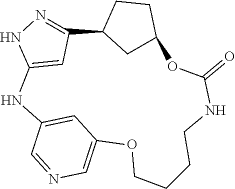 [SMILES: O=C1NCCCCOc2cncc(c2)Nc2cc(n[nH]2)[C@H]2CC[C@H](C2)O1] | (1¹S,1³R,Z)-2¹H-5,12-dioxa-3,10- diaza-4(3,5)-pyridina-2(3,5)- pyrazola-1(1,3)- cyclopentanacyclododecaphan-11- one |
| 105 | 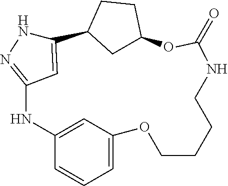 [SMILES: O=C1NCCCCOc2cccc(c2)Nc2cc([nH]n2)[C@H]2CC[C@H](C2)O1] | (1¹S,1³R,Z)-2¹H-5,12-dioxa-3,10- diaza-2(5,3)-pyrazola-4(1,3)- benzena-1(1,3)- cyclopentanacyclododecaphan-11- one |
| 106 | 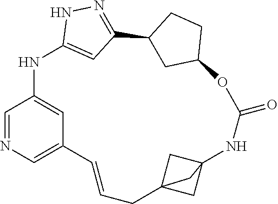 [SMILES: O=C1NC23CC(C/C=C/c4cncc(c4)Nc4cc(n[nH]4)[C@H]4CC[C@H](C4)O1)(C2)C3] | (1¹S,1³R,2⁴Z,5E)-2¹H-11-oxa-3,9- diaza-4(3,5)-pyridina-2(3,5)- pyrazola-8(1,3)- bicyclo[1.1.1]pentana-1(1,3)- cyclopentanacycloundecaphan-5- en-10-one |
| 107 | 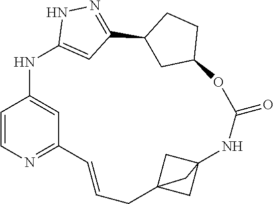 [SMILES: O=C1NC23CC(C/C=C/c4cc(ccn4)Nc4cc(n[nH]4)[C@H]4CC[C@H](C4)O1)(C2)C3] | (11S,13R,24Z,5E)-21H-11-oxa-3,9- diaza-4(4,2)-pyridina-2(3,5)- pyrazola-8(1,3)- bicyclo[1.1.1]pentana-1(1,3)- cyclopentanacycloundecaphan-5- en-10-one |
| 108 | 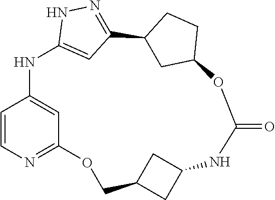 [SMILES: O=C1N[C@H]2C[C@H](COc3cc(ccn3)Nc3cc(n[nH]3)[C@H]3CC[C@H](C3)O1)C2] | (11S,13R,71R,73S,Z)-21H-5,10- dioxa-3,8-diaza-4(4,2)-pyridina- 2(3,5)-pyrazola-1(1,3)- cyclopentana-7(1,3)- cyclobutanacyclodecaphan-9-one |
| 109 | 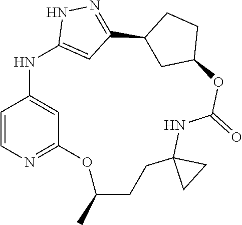 [SMILES: C[C@@H]1CCC2(CC2)NC(=O)O[C@@H]2CC[C@@H](C2)c2cc([nH]n2)Nc2ccnc(c2)O1] | (1′S,3′R,6′R,Z)-6′- methylspiro[cyclopropane-1,9′-5,12- dioxa-3,10-diaza-4(4,2)-pyridina- 2(3,5)-pyrazola-1(1,3)- cyclopentanacyclododecaphan]-11′- one |
| 110 | 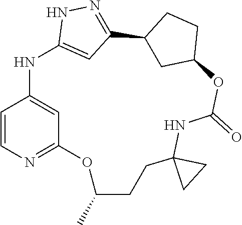 [SMILES: C[C@H]1CCC2(CC2)NC(=O)O[C@@H]2CC[C@@H](C2)c2cc([nH]n2)Nc2ccnc(c2)O1] | (1′S,3′R,6′S,Z)-6′- methylspiro[cyclopropane-1,9′-5,12- dioxa-3,10-diaza-4(4,2)-pyridina- 2(3,5)-pyrazola-1(1,3)- cyclopentanacyclododecaphan]-11′- one |
| 111 | 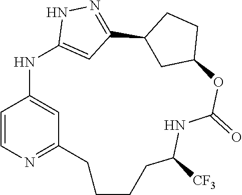 [SMILES: O=C1N[C@@H](C(F)(F)F)CCCCc2cc(ccn2)Nc2cc(n[nH]2)[C@H]2CC[C@H](C2)O1] | (1¹S,1³R,9R,Z)-9-(trifluoromethyl)- 2¹H-12-oxa-3,10-diaza-4(4,2)- pyridina-2(3,5)-pyrazola-1(1,3)- cyclopentanacyclododecaphan-11- one |
| 112 | 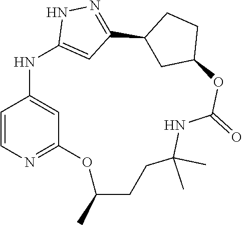 [SMILES: C[C@@H]1CCC(C)(C)NC(=O)O[C@@H]2CC[C@@H](C2)c2cc([nH]n2)Nc2ccnc(c2)O1] | (1¹S,1³R,6R,Z)-6,9,9-trimethyl-2¹H- 5,12-dioxa-3,10-diaza-4(4,2)- pyridina-2(3,5)-pyrazola-1(1,3)- cyclopentanacyclododecaphan-11- one |
| 113 | 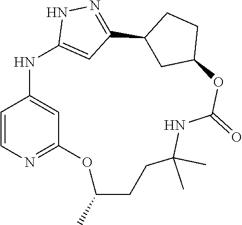 [SMILES: C[C@H]1CCC(C)(C)NC(=O)O[C@@H]2CC[C@@H](C2)c2cc([nH]n2)Nc2ccnc(c2)O1] | (1¹S,1³R,6S,Z)-6,9,9-trimethyl-2¹H- 5,12-dioxa-3,10-diaza-4(4,2)- pyridina-2(3,5)-pyrazola-1(1,3)- cyclopentanacyclododecaphan-11- one |
| 114 | 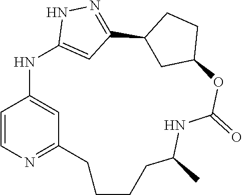 [SMILES: C[C@H]1CCCCc2cc(ccn2)Nc2cc(n[nH]2)[C@H]2CC[C@H](C2)OC(=O)N1] | (1¹S,1³R,9S,Z)-9-methyl-2¹H-12- oxa-3,10-diaza-4(4,2)-pyridina- 2(3,5)-pyrazola-1(1,3)- cyclopentanacyclododecaphan-11- one |
| 115 | 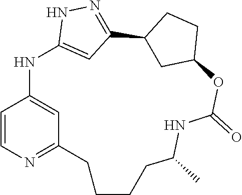 [SMILES: C[C@@H]1CCCCc2cc(ccn2)Nc2cc(n[nH]2)[C@H]2CC[C@H](C2)OC(=O)N1] | (1¹S,1³R,9R,Z)-9-methyl-2¹H-12- oxa-3,10-diaza-4(4,2)-pyridina- 2(3,5)-pyrazola-1(1,3)- cyclopentanacyclododecaphan-11- one |
| 116 | 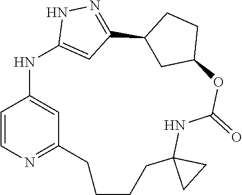 [SMILES: O=C1NC2(CCCCc3cc(ccn3)Nc3cc(n[nH]3)[C@H]3CC[C@H](C3)O1)CC2] | (1′S,3′R,Z)-spiro[cyclopropane-1,9′- 12-oxa-3,10-diaza-4(4,2)-pyridina- 2(3,5)-pyrazola-1(1,3)- cyclopentanacyclododecaphan]-11′- one |
| 117 | 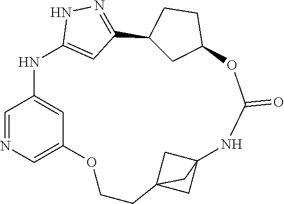 [SMILES: O=C1NC23CC(CCOc4cncc(c4)Nc4cc(n[nH]4)[C@H]4CC[C@H](C4)O1)(C2)C3] | (1¹S,1³R,Z)-2¹H-5,11-dioxa-3,9- diaza-4(3,5)-pyridina-2(3,5)- pyrazola-8(1,3)- bicyclo[1.1.1]pentana-1(1,3)- cyclopentanacycloundecaphan-10- one |
| 118 | 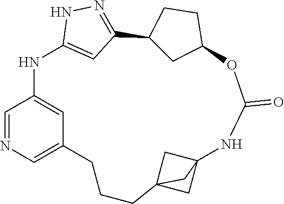 [SMILES: O=C1NC23CC(CCCc4cncc(c4)Nc4cc(n[nH]4)[C@H]4CC[C@H](C4)O1)(C2)C3] | (1¹S,1³R,Z)-2¹H-11-oxa-3,9-diaza- 4(3,5)-pyridina-2(3,5)-pyrazola- 8(1,3)-bicyclo[1.1.1]pentana-1(1,3)- cyclopentanacycloundecaphan-10- one |
| 119 | 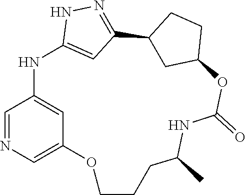 [SMILES: C[C@H]1CCCOc2cncc(c2)Nc2cc(n[nH]2)[C@H]2CC[C@H](C2)OC(=O)N1] | (1¹S,1³R,9S,Z)-9-methyl-2¹H-5,12- dioxa-3,10-diaza-4(3,5)-pyridina- 2(3,5)-pyrazola-1(1,3)- cyclopentanacyclododecaphan-11- one |
| 120 | 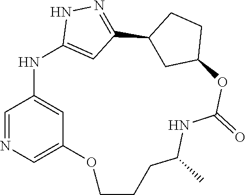 [SMILES: C[C@@H]1CCCOc2cncc(c2)Nc2cc(n[nH]2)[C@H]2CC[C@H](C2)OC(=O)N1] | (1¹S,1³R,9R,Z)-9-methyl-2¹H-5,12- dioxa-3,10-diaza-4(3,5)-pyridina- 2(3,5)-pyrazola-1(1,3)- cyclopentanacyclododecaphan-11- one |
| 121 | 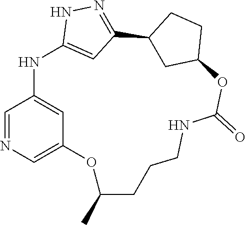 [SMILES: C[C@@H]1CCCNC(=O)O[C@@H]2CC[C@@H](C2)c2cc([nH]n2)Nc2cncc(c2)O1] | (1¹S,1³R,6R,Z)-6-methyl-2¹H-5,12- dioxa-3,10-diaza-4(3,5)-pyridina- 2(3,5)-pyrazola-1(1,3)- cyclopentanacyclododecaphan-11- one |
| 122 | 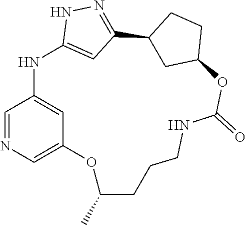 [SMILES: C[C@H]1CCCNC(=O)O[C@@H]2CC[C@@H](C2)c2cc([nH]n2)Nc2cncc(c2)O1] | (1¹S,1³R,6S,Z)-6-methyl-2¹H-5,12- dioxa-3,10-diaza-4(3,5)-pyridina- 2(3,5)-pyrazola-1(1,3)- cyclopentanacyclododecaphan-11- one |
| 123 | 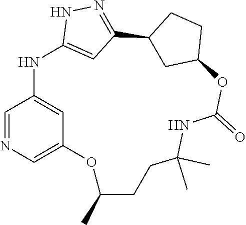 [SMILES: C[C@@H]1CCC(C)(C)NC(=O)O[C@@H]2CC[C@@H](C2)c2cc([nH]n2)Nc2cncc(c2)O1] | (1¹S,1³R,6R,Z)-6,9,9-trimethyl-2¹H- 5,12-dioxa-3,10-diaza-4(3,5)- pyridina-2(3,5)-pyrazola-1(1,3)- cyclopentanacyclododecaphan-11- one |
| 124 | 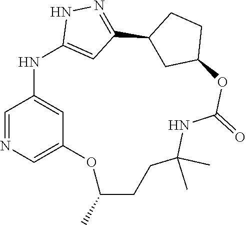 [SMILES: C[C@H]1CCC(C)(C)NC(=O)O[C@@H]2CC[C@@H](C2)c2cc([nH]n2)Nc2cncc(c2)O1] | (1¹S,1³R,6S,Z)-6,9,9-trimethyl-2¹H- 5,12-dioxa-3,10-diaza-4(3,5)- pyridina-2(3,5)-pyrazola-1(1,3)- cyclopentanacyclododecaphan-11- one |
| 125 | 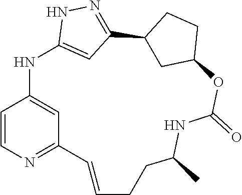 [SMILES: C[C@H]1CC/C=C/c2cc(ccn2)Nc2cc(n[nH]2)[C@H]2CC[C@H](C2)OC(=O)N1] | (1¹S,1³R,2⁴Z,5E,9S)-9-methyl-2¹H- 12-oxa-3,10-diaza-4(4,2)-pyridina- 2(3,5)-pyrazola-1(1,3)- cyclopentanacyclododecaphan-5- en-11-one |
| 126 | 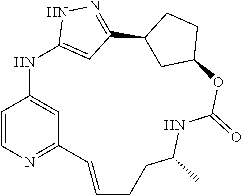 [SMILES: C[C@@H]1CC/C=C/c2cc(ccn2)Nc2cc(n[nH]2)[C@H]2CC[C@H](C2)OC(=O)N1] | (1¹S,1³R,2⁴Z,5E,9R)-9-methyl-2¹H- 12-oxa-3,10-diaza-4(4,2)-pyridina- 2(3,5)-pyrazola-1(1,3)- cyclopentanacyclododecaphan-5- en-11-one |
| 127 | 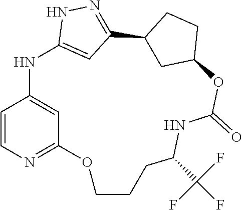 [SMILES: O=C1N[C@H](C(F)(F)F)CCCOc2cc(ccn2)Nc2cc(n[nH]2)[C@H]2CC[C@H](C2)O1] | (1¹S,1³R,9S,Z)-9-(trifluoromethyl)- 2¹H-5,12-dioxa-3,10-diaza-4(4,2)- pyridina-2(3,5)-pyrazola-1(1,3)- cyclopentanacyclododecaphan-11- one |
| 128 | 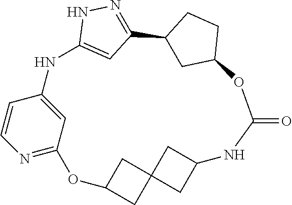 [SMILES: O=C1NC2CC3(C2)CC(C3)Oc2cc(ccn2)Nc2cc(n[nH]2)[C@H]2CC[C@H](C2)O1] | (6¹S,6³R,Z)-5¹H-2,7-dioxa-4,9- diaza-3(2,4)-pyridina-5(5,3)- pyrazola-6(1,3)-cyclopentana- 1,10(1,3)-dicyclobutanadecaphan-8- one |
| 129 | 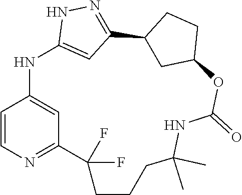 [SMILES: CC1(C)CCCC(F)(F)c2cc(ccn2)Nc2cc(n[nH]2)[C@H]2CC[C@H](C2)OC(=O)N1] | (1¹S,1³R,Z)-5,5-difluoro-9,9- dimethyl-2¹H-12-oxa-3,10-diaza- 4(4,2)-pyridina-2(3,5)-pyrazola- 1(1,3)- cyclopentanacyclododecaphan-11- one |
| 130 | 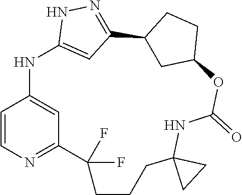 [SMILES: O=C1NC2(CCCC(F)(F)c3cc(ccn3)Nc3cc(n[nH]3)[C@H]3CC[C@H](C3)O1)CC2] | (1′S,3′R,Z)-5′,5′- difluorospiro[cyclopropane-1,9′-12- oxa-3,10-diaza-4(4,2)-pyridina- 2(3,5)-pyrazola-1(1,3)- cyclopentanacyclododecaphan]-11′- one |
| 131 | 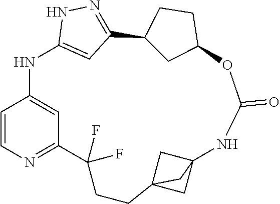 [SMILES: O=C1NC23CC(CCC(F)(F)c4cc(ccn4)Nc4cc(n[nH]4)[C@H]4CC[C@H](C4)O1)(C2)C3] | (1¹S,1³R,Z)-5,5-difluoro-2¹H-11- oxa-3,9-diaza-4(4,2)-pyridina- 2(3,5)-pyrazola-8(1,3)- bicyclo[1.1.1]pentana-1(1,3)- cyclopentanacycloundecaphan-10- one |
| 132 | 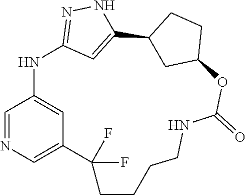 [SMILES: O=C1NCCCCC(F)(F)c2cncc(c2)Nc2cc([nH]n2)[C@H]2CC[C@H](C2)O1] | (1¹S,1³R,Z)-5,5-difluoro-2¹H-12- oxa-3,10-diaza-4(3,5)-pyridina- 2(5,3)-pyrazola-1(1,3)- cyclopentanacyclododecaphan-11- one |
| 133 | 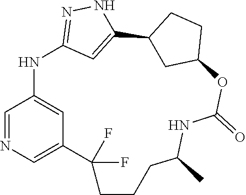 [SMILES: C[C@H]1CCCC(F)(F)c2cncc(c2)Nc2cc([nH]n2)[C@H]2CC[C@H](C2)OC(=O)N1] | (1¹S,1³R,9S,Z)-5,5-difluoro-9- methyl-2¹H-12-oxa-3,10-diaza- 4(3,5)-pyridina-2(5,3)-pyrazola- 1(1,3)- cyclopentanacyclododecaphan-11- one |
| 134 | 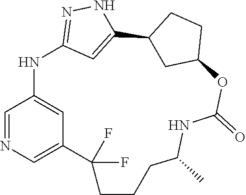 [SMILES: C[C@@H]1CCCC(F)(F)c2cncc(c2)Nc2cc([nH]n2)[C@H]2CC[C@H](C2)OC(=O)N1] | (1¹S,1³R,9R,Z)-5,5-difluoro-9- methyl-2¹H-12-oxa-3,10-diaza- 4(3,5)-pyridina-2(5,3)-pyrazola- 1(1,3)- cyclopentanacyclododecaphan-11- one |
| 135 | 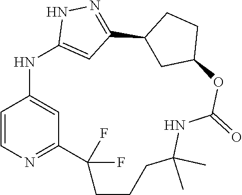 [SMILES: CC1(C)CCCC(F)(F)c2cc(ccn2)Nc2cc(n[nH]2)[C@H]2CC[C@H](C2)OC(=O)N1] | (1¹S,1³R,Z)-5,5-difluoro-9,9- dimethyl-2¹H-12-oxa-3,10-diaza- 4(4,2)-pyridina-2(3,5)-pyrazola- 1(1,3)- cyclopentanacyclododecaphan-11- one |
| 136 | 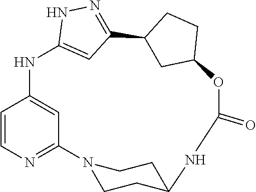 [SMILES: O=C1NC2CCN(CC2)c2cc(ccn2)Nc2cc(n[nH]2)[C@H]2CC[C@H](C2)O1] | (5¹S,5³R,Z)-4¹H-6-oxa-3,8-diaza- 2(2,4)-pyridina-1(1,4)-piperidina- 4(5,3)-pyrazola-5(1,3)- cyclopentanacyclooctaphan-7-one |
| 137 | 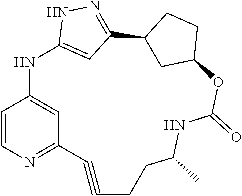 [SMILES: C[C@@H]1CCC#Cc2cc(ccn2)Nc2cc(n[nH]2)[C@H]2CC[C@H](C2)OC(=O)N1] | (1¹S,1³R,2⁴Z,9R)-9-methyl-2¹H-12- oxa-3,10-diaza-4(4,2)-pyridina- 2(3,5)-pyrazola-1(1,3)- cyclopentanacyclododecaphan-5- yn-11-one |
| 138 | 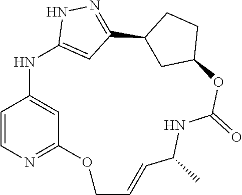 [SMILES: C[C@@H]1/C=C/COc2cc(ccn2)Nc2cc(n[nH]2)[C@H]2CC[C@H](C2)OC(=O)N1] | (1¹S,1³R,2⁴Z,7E,9R)-9-methyl-2¹H- 5,12-dioxa-3,10-diaza-4(4,2)- pyridina-2(3,5)-pyrazola-1(1,3)- cyclopentanacyclododecaphan-7- en-11-one |
| 139 | 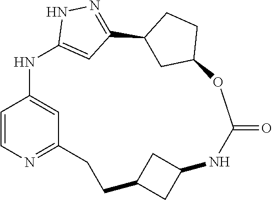 | (1¹S,1³R,7¹S,7³S,Z)-2¹H-10-oxa- 3,8-diaza-4(4,2)-pyridina-2(3,5)- pyrazola-1(1,3)-cyclopentana- 7(1,3)-cyclobutanacyclodecaphan- 9-one |
| 140 | 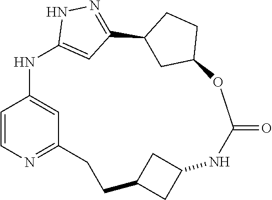 | (1¹S,1³R,7¹R,7³R,Z)-2¹H-10-oxa- 3,8-diaza-4(4,2)-pyridina-2(3,5)- pyrazola-1(1,3)-cyclopentana- 7(1,3)-cyclobutanacyclodecaphan- 9-one |
| 141 | 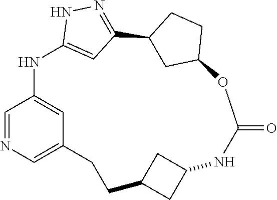 | (1¹S,1³R,7¹R,7³R,Z)-2¹H-10-oxa- 3,8-diaza-4(3,5)-pyridina-2(3,5)- pyrazola-1(1,3)-cyclopentana- 7(1,3)-cyclobutanacyclodecaphan- 9-one |
| 142 | 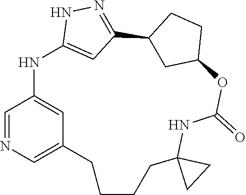 [SMILES: O=C1NC2(CCCCc3cncc(c3)Nc3cc(n[nH]3)[C@H]3CC[C@H](C3)O1)CC2] | (1′S,3′R,Z)-spiro[cyclopropane-1,9′- 12-oxa-3,10-diaza-4(3,5)-pyridina- 2(3,5)-pyrazola-1(1,3)- cyclopentanacyclododecaphan]-11′- one |
| 143 | 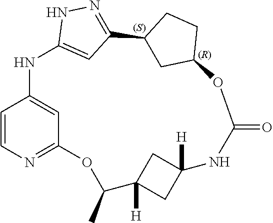 | (1¹S,1³R,7¹S,7³S,6R,Z)-6-methyl- 2¹H-5,10-dioxa-3,8-diaza-4(4,2)- pyridina-2(3,5)-pyrazola-1(1,3)- cyclopentana-7(1,3)- cyclobutanacyclodecaphan-9-one |
| 144 | 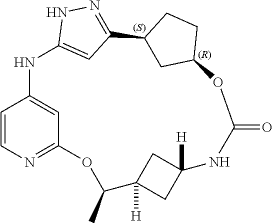 | (1¹S,1³R,7¹R,7³R,6R,Z)-6-methyl- 2¹H-5,10-dioxa-3,8-diaza-4(4,2)- pyridina-2(3,5)-pyrazola-1(1,3)- cyclopentana-7(1,3)- cyclobutanacyclodecaphan-9-one |
| 145 | 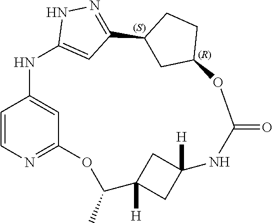 | (1¹S,1³R,7¹S,7³R,6S,Z)-6-methyl- 2¹H-5,10-dioxa-3,8-diaza-4(4,2)- pyridina-2(3,5)-pyrazola-1(1,3)- cyclopentana-7(1,3)- cyclobutanacyclodecaphan-9-one |
| 146 | 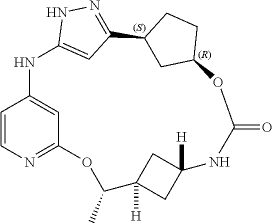 | (1¹S,1³R,7¹R,7³S,6S,Z)-6-methyl- 2¹H-5,10-dioxa-3,8-diaza-4(4,2)- pyridina-2(3,5)-pyrazola-1(1,3)- cyclopentana-7(1,3)- cyclobutanacyclodecaphan-9-one |
| 147 | 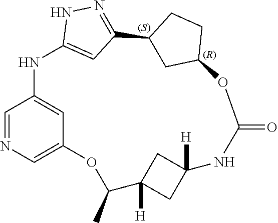 | (1¹S,1³R,7¹S,7³S,6R,Z)-6-methyl- 2¹H-5,10-dioxa-3,8-diaza-4(3,5)- pyridina-2(3,5)-pyrazola-1(1,3)- cyclopentana-7(1,3)- cyclobutanacyclodecaphan-9-one |
| 148 | 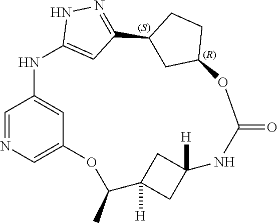 | (1¹S,1³R,7¹R,7³R,6R,Z)-6-methyl- 2¹H-5,10-dioxa-3,8-diaza-4(3,5)- pyridina-2(3,5)-pyrazola-1(1,3)- cyclopentana-7(1,3)- cyclobutanacyclodecaphan-9-one |
| 149 | 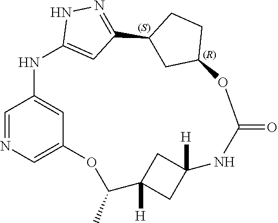 | (1¹S,1³R,7¹S,7³R,6S,Z)-6-methyl- 2¹H-5,10-dioxa-3,8-diaza-4(3,5)- pyridina-2(3,5)-pyrazola-1(1,3)- cyclopentana-7(1,3)- cyclobutanacyclodecaphan-9-one |
| 150 | 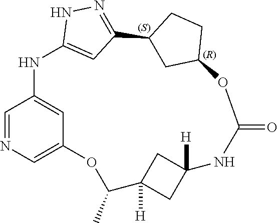 | (1¹S,1³R,7¹R,7³S,6S,Z)-6-methyl- 2¹H-5,10-dioxa-3,8-diaza-4(3,5)- pyridina-2(3,5)-pyrazola-1(1,3)- cyclopentana-7(1,3)- cyclobutanacyclodecaphan-9-one |
| 151 | 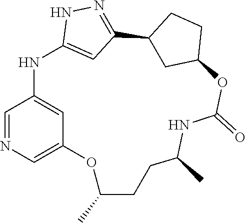 [SMILES: C[C@H]1CC[C@H](C)Oc2cncc(c2)Nc2cc(n[nH]2)[C@H]2CC[C@H](C2)OC(=O)N1] | (1¹S,1³R,6S,9S,Z)-6,9-dimethyl- 2¹H-5,12-dioxa-3,10-diaza-4(3,5)- pyridina-2(3,5)-pyrazola-1(1,3)- cyclopentanacyclododecaphan-11- one |
| 152 | 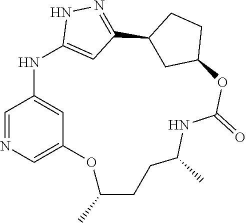 [SMILES: C[C@@H]1CC[C@H](C)Oc2cncc(c2)Nc2cc(n[nH]2)[C@H]2CC[C@H](C2)OC(=O)N1] | (1¹S,1³R,6S,9R,Z)-6,9-dimethyl- 2¹H-5,12-dioxa-3,10-diaza-4(3,5)- pyridina-2(3,5)-pyrazola-1(1,3)- cyclopentanacyclododecaphan-11- one |
| 153 | 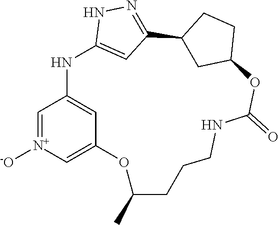 | (1¹S,1³R,6R,Z)-6-methyl-11-oxo- 2¹H-5,12-dioxa-3,10-diaza-4(3,5)- pyridin-1-iuma-2(3,5)-pyrazola- 1(1,3)- cyclopentanacyclododecaphane 41- oxide |
| 154 | 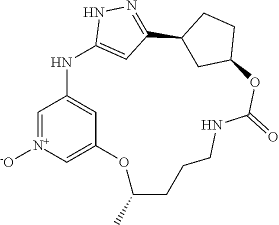 | (1¹S,1³R,6S,Z)-6-methyl-11-oxo- 2¹H-5,12-dioxa-3,10-diaza-4(3,5)- pyridin-1-iuma-2(3,5)-pyrazola- 1(1,3)- cyclopentanacyclododecaphane 41- oxide |
| 155 | 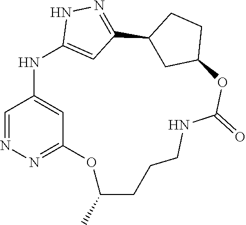 [SMILES: C[C@H]1CCCNC(=O)O[C@@H]2CC[C@@H](C2)c2cc([nH]n2)Nc2cnnc(c2)O1] | (1¹S,1³R,6S,Z)-6-methyl-2¹H-5,12- dioxa-3,10-diaza-4(5,3)-pyridazina- 2(3,5)-pyrazola-1(1,3)- cyclopentanacyclododecaphan-11- one |
| 156 | 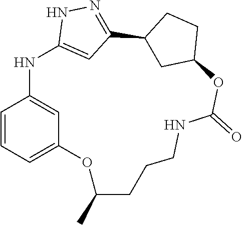 [SMILES: C[C@@H]1CCCNC(=O)O[C@@H]2CC[C@@H](C2)c2cc([nH]n2)Nc2cccc(c2)O1] | (1¹S,1³R,6R,Z)-6-methyl-2¹H-5,12- dioxa-3,10-diaza-2(3,5)-pyrazola- 4(1,3)-benzena-1(1,3)- cyclopentanacyclododecaphan-11- one |
| 157 | 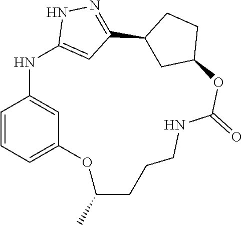 [SMILES: C[C@H]1CCCNC(=O)O[C@@H]2CC[C@@H](C2)c2cc([nH]n2)Nc2cccc(c2)O1] | (1¹S,1³R,6S,Z)-6-methyl-2¹H-5,12- dioxa-3,10-diaza-2(3,5)-pyrazola- 4(1,3)-benzena-1(1,3)- cyclopentanacyclododecaphan-11- one |
| 158 | 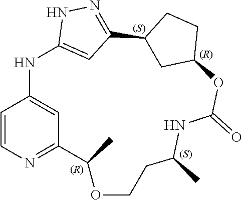 | (1¹S,1³R,5R,9S,Z)-5,9-dimethyl- 2¹H-6,12-dioxa-3,10-diaza-4(4,2)- pyridina-2(3,5)-pyrazola-1(1,3)- cyclopentanacyclododecaphan-11- one |
| 159 | 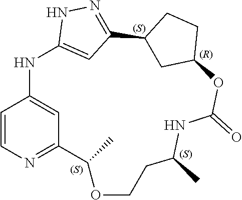 | (1¹S,1³R,5S,9S,Z)-5,9-dimethyl- 2¹H-6,12-dioxa-3,10-diaza-4(4,2)- pyridina-2(3,5)-pyrazola-1(1,3)- cyclopentanacyclododecaphan-11- one |
| 160 | 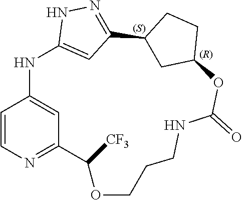 | (1¹S,1³R,5S,Z)-5-(trifluoromethyl)- 2¹H-6,12-dioxa-3,10-diaza-4(4,2)- pyridina-2(3,5)-pyrazola-1(1,3)- cyclopentanacyclododecaphan-11- one |
| 161 | 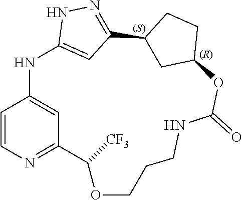 | (1¹S,1³R,5R,Z)-5-(trifluoromethyl)- 2¹H-6,12-dioxa-3,10-diaza-4(4,2)- pyridina-2(3,5)-pyrazola-1(1,3)- cyclopentanacyclododecaphan-11- one |
| 162 | 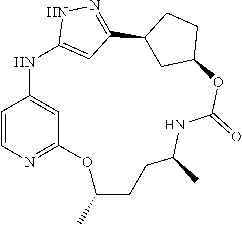 [SMILES: C[C@H]1CC[C@H](C)Oc2cc(ccn2)Nc2cc(n[nH]2)[C@H]2CC[C@H](C2)OC(=O)N1] | (1¹S,1³R,6S,9S,Z)-6,9-dimethyl- 2¹H-5,12-dioxa-3,10-diaza-4(4,2)- pyridina-2(3,5)-pyrazola-1(1,3)- cyclopentanacyclododecaphan-11- one |
| 163 | 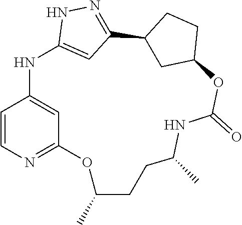 [SMILES: C[C@@H]1CC[C@H](C)Oc2cc(ccn2)Nc2cc(n[nH]2)[C@H]2CC[C@H](C2)OC(=O)N1] | (1¹S,1³R,6S,9R,Z)-6,9-dimethyl- 2¹H-5,12-dioxa-3,10-diaza-4(4,2)- pyridina-2(3,5)-pyrazola-1(1,3)- cyclopentanacyclododecaphan-11- one |
| 164 | 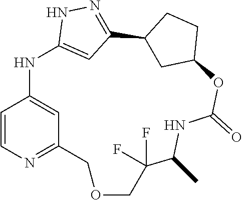 [SMILES: C[C@@H]1NC(=O)O[C@@H]2CC[C@@H](C2)c2cc([nH]n2)Nc2ccnc(c2)COCC1(F)F] | (1¹S,1³R,9S,Z)-8,8-difluoro-9- methyl-2¹H-6,12-dioxa-3,10-diaza- 4(4,2)-pyridina-2(3,5)-pyrazola- 1(1,3)- cyclopentanacyclododecaphan-11- one |
| 165 | 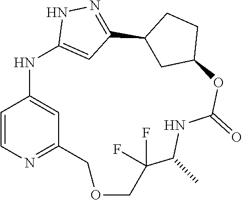 [SMILES: C[C@H]1NC(=O)O[C@@H]2CC[C@@H](C2)c2cc([nH]n2)Nc2ccnc(c2)COCC1(F)F] | (1¹S,1³R,9R,Z)-8,8-difluoro-9- methyl-2¹H-6,12-dioxa-3,10-diaza- 4(4,2)-pyridina-2(3,5)-pyrazola- 1(1,3)- cyclopentanacyclododecaphan-11- one |
| 166 | 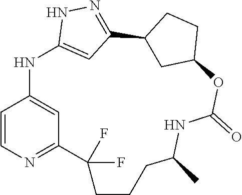 [SMILES: C[C@H]1CCCC(F)(F)c2cc(ccn2)Nc2cc(n[nH]2)[C@H]2CC[C@H](C2)OC(=O)N1] | (1¹S,1³R,9S,Z)-5,5-difluoro-9- methyl-2¹H-12-oxa-3,10-diaza- 4(4,2)-pyridina-2(3,5)-pyrazola- 1(1,3)- cyclopentanacyclododecaphan-11- one |
| 167 | 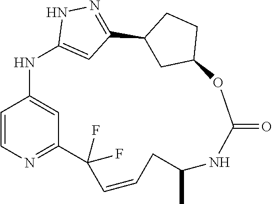 [SMILES: C[C@H]1C/C=C\C(F)(F)c2cc(ccn2)Nc2cc(n[nH]2)[C@H]2CC[C@H](C2)OC(=O)N1] | (1¹S,1³R,2⁴Z,6Z,9S)-5,5-difluoro-9- methyl-2¹H-12-oxa-3,10-diaza- 4(4,2)-pyridina-2(3,5)-pyrazola- 1(1,3)- cyclopentanacyclododecaphan-6- en-11-one |
| 168 | 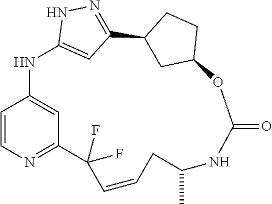 [SMILES: C[C@@H]1C/C=C\C(F)(F)c2cc(ccn2)Nc2cc(n[nH]2)[C@H]2CC[C@H](C2)OC(=O)N1] | (1¹S,1³R,2⁴Z,6Z,9R)-5,5-difluoro-9- methyl-2¹H-12-oxa-3,10-diaza- 4(4,2)-pyridina-2(3,5)-pyrazola- 1(1,3)- cyclopentanacyclododecaphan-6- en-11-one |
| 169 | 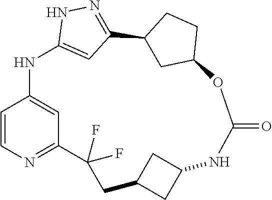 | (1¹S,1³R,7¹R,7³S,Z)-5,5-difluoro- 2¹H-10-oxa-3,8-diaza-4(4,2)- pyridina-2(3,5)-pyrazola-1(1,3)- cyclopentana-7(1,3)- cyclobutanacyclodecaphan-9-one |
| 170 | 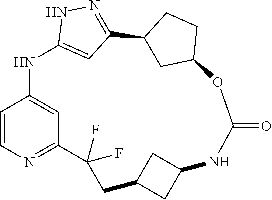 | (1¹S,1³R,7¹S,7³R,Z)-5,5-difluoro- 2¹H-10-oxa-3,8-diaza-4(4,2)- pyridina-2(3,5)-pyrazola-1(1,3)- cyclopentana-7(1,3)- cyclobutanacyclodecaphan-9-one |
| 171 | 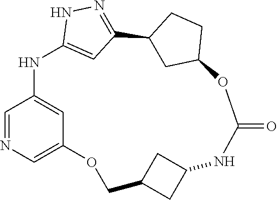 | (1¹S,1³R,7¹R,7³S,Z)-2¹H-5,10- dioxa-3,8-diaza-4(3,5)-pyridina- 2(3,5)-pyrazola-1(1,3)- cyclopentana-7(1,3)- cyclobutanacyclodecaphan-9-one |
| 172 | 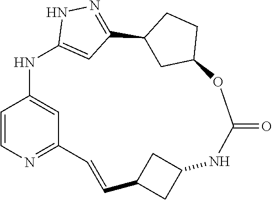 | (1¹S,1³R,2⁴Z,7¹R,7³S,5E)-2¹H-10- oxa-3,8-diaza-4(4,2)-pyridina- 2(3,5)-pyrazola-1(1,3)- cyclopentana-7(1,3)- cyclobutanacyclodecaphan-5-en-9- one |
| 173 | 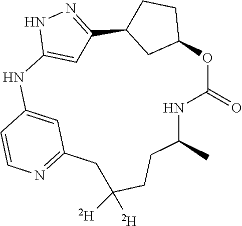 [SMILES: [2H]C1([2H])CC[C@H](C)NC(=O)O[C@@H]2CC[C@@H](C2)c2cc([nH]n2)Nc2ccnc(c2)C1] | (1¹S,1³R,9S,Z)-9-methyl-2¹H-5,12- dioxa-3,10-diaza-4(4,2)-pyridina- 2(3,5)-pyrazola-1(1,3)- cyclopentanacyclododecaphan-11- one-6,6-d2 |
| 174 | 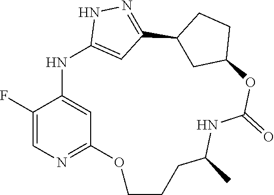 [SMILES: C[C@H]1CCCOc2cc(c(F)cn2)Nc2cc(n[nH]2)[C@H]2CC[C@H](C2)OC(=O)N1] | (1¹S,1³R,9S,Z)-4⁵-fluoro-9-methyl- 2¹H-5,12-dioxa-3,10-diaza-4(4,2)- pyridina-2(3,5)-pyrazola-1(1,3)- cyclopentanacyclododecaphan-11- one |
| 175 | 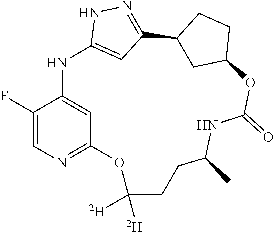 [SMILES: [2H]C1([2H])CC[C@H](C)NC(=O)O[C@@H]2CC[C@@H](C2)c2cc([nH]n2)Nc2cc(ncc2F)O1] | (1¹S,1³R,9S,Z)-4⁵-fluoro-9-methyl- 2¹H-5,12-dioxa-3,10-diaza-4(4,2)- pyridina-2(3,5)-pyrazola-1(1,3)- cyclopentanacyclododecaphan-11- one-6,6-d₂ |
| 176 | 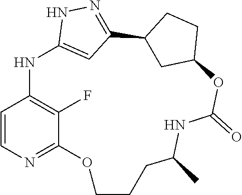 [SMILES: C[C@H]1CCCOc2nccc(c2F)Nc2cc(n[nH]2)[C@H]2CC[C@H](C2)OC(=O)N1] | (1¹S,1³R,9S,Z)-4³-fluoro-9-methyl- 2¹H-5,12-dioxa-3,10-diaza-4(4,2)- pyridina-2(3,5)-pyrazola-1(1,3)- cyclopentanacyclododecaphan-11- one |
| 177 | 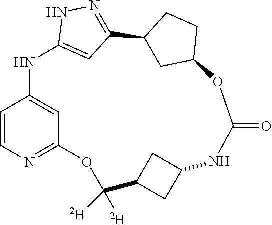 | (1¹S,1³R,7¹R,7³S,Z)-2¹H-5,10- dioxa-3,8-diaza-4(4,2)-pyridina- 2(3,5)-pyrazola-1(1,3)- cyclopentana-7(1,3)- cyclobutanacyclodecaphan-9-one- 6,6-d₂ |
| 178 | 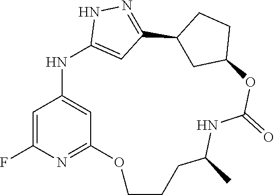 [SMILES: C[C@H]1CCCOc2cc(cc(F)n2)Nc2cc(n[nH]2)[C@H]2CC[C@H](C2)OC(=O)N1] | (1¹S,1³R,9S,Z)-4⁶-fluoro-9-methyl- 2¹H-5,12-dioxa-3,10-diaza-4(4,2)- pyridina-2(3,5)-pyrazola-1(1,3)- cyclopentanacyclododecaphan-11- one |
| 179 | 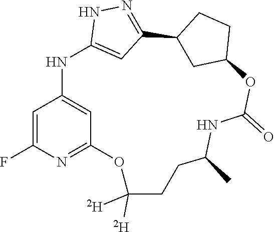 [SMILES: [2H]C1([2H])CC[C@H](C)NC(=O)O[C@@H]2CC[C@@H](C2)c2cc([nH]n2)Nc2cc(F)nc(c2)O1] | (1¹S,1³R,9S,Z)-4⁶-fluoro-9-methyl- 2¹H-5,12-dioxa-3,10-diaza-4(4,2)- pyridina-2(3,5)-pyrazola-1(1,3)- cyclopentanacyclododecaphan-11- one-6,6-d₂ |
| 180 | 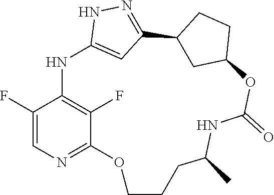 [SMILES: C[C@H]1CCCOc2ncc(F)c(c2F)Nc2cc(n[nH]2)[C@H]2CC[C@H](C2)OC(=O)N1] | (1¹S,1³R,9S,Z)-4³,4⁵-difluoro-9- methyl-2¹H-5,12-dioxa-3,10-diaza- 4(4,2)-pyridina-2(3,5)-pyrazola- 1(1,3)-cyclopentanacyclododecaphan- 11-one |
| 181 | 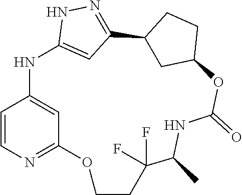 [SMILES: C[C@@H]1NC(=O)O[C@@H]2CC[C@@H](C2)c2cc([nH]n2)Nc2ccnc(c2)OCCC1(F)F] | (1¹S,1³R,9S,Z)-8,8-difluoro-9- methyl-2¹H-5,12-dioxa-3,10-diaza- 4(4,2)-pyridina-2(3,5)-pyrazola- 1(1,3)- cyclopentanacyclododecaphan-11- one |
| 182 | 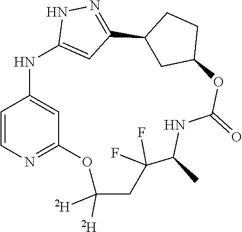 [SMILES: [2H]C1([2H])CC(F)(F)[C@H](C)NC(=O)O[C@@H]2CC[C@@H](C2)c2cc([nH]n2)Nc2ccnc(c2)O1] | (1¹S,1³R,9S,Z)-8,8-difluoro-9- methyl-2¹H-5,12-dioxa-3,10-diaza- 4(4,2)-pyridina-2(3,5)-pyrazola- 1(1,3)- cyclopentanacyclododecaphan-11- one-6,6-d₂ |
| 183 | 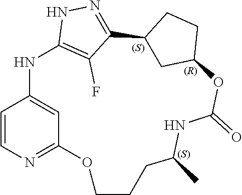 | (1¹S,1³R,9S,E)-2⁴-fluoro-9-methyl- 2¹H-5,12-dioxa-3,10-diaza-4(4,2)- pyridina-2(3,5)-pyrazola-1(1,3)- cyclopentanacyclododecaphan-11- one |
| 184 | 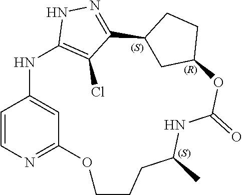 | (1¹S,1³R,9S,E)-2⁴-chloro-9-methyl- 2¹H-5,12-dioxa-3,10-diaza-4(4,2)- pyridina-2(3,5)-pyrazola-1(1,3)- cyclopentanacyclododecaphan-11- one |
| 185 | 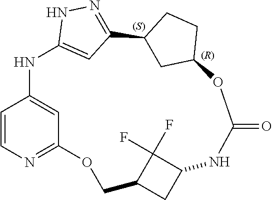 | (1¹S,1³R,7¹S,7³R,Z)-7²,7²-difluoro- 2¹H-5,10-dioxa-3,8-diaza-4(4,2)- pyridina-2(3,5)-pyrazola-1(1,3)- cyclopentana-7(1,3)- cyclobutanacyclodecaphan-9-one |
| 186 | 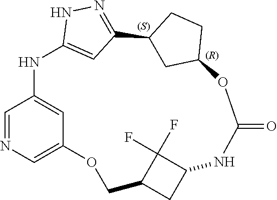 | (1¹S,1³R,7¹S,7³R,Z)-7²,7²-difluoro- 2¹H-5,10-dioxa-3,8-diaza-4(3,5)- pyridina-2(3,5)-pyrazola-1(1,3)- cyclopentana-7(1,3)- cyclobutanacyclodecaphan-9-one |
| 187 | 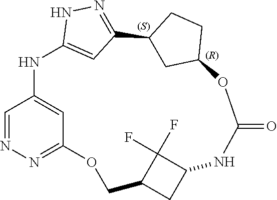 | (1¹S,1³R,7¹S,7³R,Z)-7²,7²-difluoro- 2¹H-5,10-dioxa-3,8-diaza-4(5,3)- pyridazina-2(3,5)-pyrazola-1(1,3)- cyclopentana-7(1,3)- cyclobutanacyclodecaphan-9-one |
| 188 | 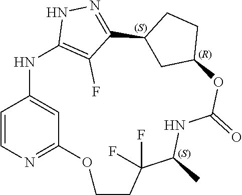 | (1¹S,1³R,9S,E)-2⁴,8,8-trifluoro-9- methyl-2¹H-5,12-dioxa-3,10-diaza- 4(4,2)-pyridina-2(3,5)-pyrazola- 1(1,3)- cyclopentanacyclododecaphan-11- one |
| 189 | 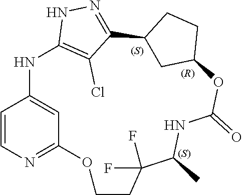 | (1¹S,1³R,9S,E)-2⁴-chloro-8,8- difluoro-9-methyl-2¹H-5,12-dioxa- 3,10-diaza-4(4,2)-pyridina-2(3,5)- pyrazola-1(1,3)- cyclopentanacyclododecaphan-11- one |
| 190 | 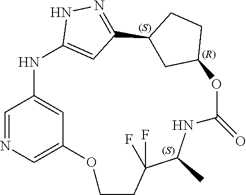 | (1¹S,1³R,9S,Z)-8,8-difluoro-9- methyl-2¹H-5,12-dioxa-3,10-diaza- 4(3,5)-pyridina-2(3,5)-pyrazola- 1(1,3)- cyclopentanacyclododecaphan-11- one |
| 191 | 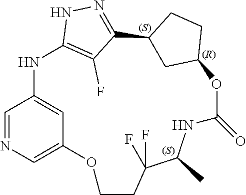 | (1¹S,1³R,9S,E)-2⁴,8,8-trifluoro-9- methyl-2¹H-5,12-dioxa-3,10-diaza- 4(3,5)-pyridina-2(3,5)-pyrazola- 1(1,3)- cyclopentanacyclododecaphan-11- one |
| 192 | 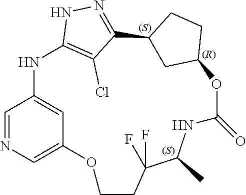 | (1¹S,1³R,9S,E)-2⁴-chloro-8,8- difluoro-9-methyl-2¹H-5,12-dioxa- 3,10-diaza-4(3,5)-pyridina-2(3,5)- pyrazola-1(1,3)- cyclopentanacyclododecaphan-11- one |
| 193 | 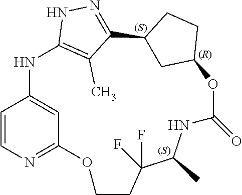 | (1¹S,1³R,9S,Z)-8,8-difluoro-2⁴,9- dimethyl-2¹H-5,12-dioxa-3,10-diaza- 4(4,2)-pyridina-2(3,5)-pyrazola- 1(1,3)- cyclopentanacyclododecaphan-11- one |
| 194 | 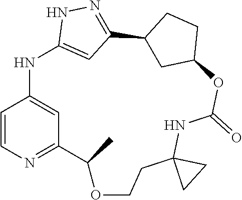 [SMILES: C[C@H]1OCCC2(CC2)NC(=O)O[C@@H]2CC[C@@H](C2)c2cc([nH]n2)Nc2ccnc1c2] | (1′S,3′R,5′R,Z)-5′- methylspiro[cyclopropane-1,9′-6,12- dioxa-3,10-diaza-4(4,2)-pyridina- 2(3,5)-pyrazola-1(1,3)- cyclopentanacyclododecaphan]-11′- one |
| 195 | 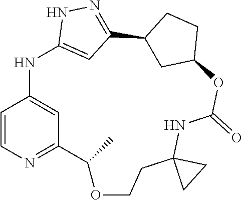 [SMILES: C[C@@H]1OCCC2(CC2)NC(=O)O[C@@H]2CC[C@@H](C2)c2cc([nH]n2)Nc2ccnc1c2] | (1′S,3′R,5′S,Z)-5′- methylspiro[cyclopropane-1,9′-6,12- dioxa-3,10-diaza-4(4,2)-pyridina- 2(3,5)-pyrazola-1(1,3)- cyclopentanacyclododecaphan]-11′- one |
| 196 | 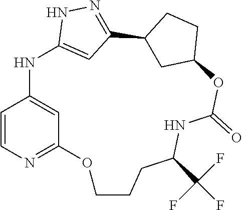 [SMILES: O=C1N[C@@H](C(F)(F)F)CCCOc2cc(ccn2)Nc2cc(n[nH]2)[C@H]2CC[C@H](C2)O1] | (1¹S,1³R,9R,Z)-9-(trifluoromethyl)- 2¹H-5,12-dioxa-3,10-diaza-4(4,2)- pyridina-2(3,5)-pyrazola-1(1,3)- cyclopentanacyclododecaphan-11- one |
| 197 | 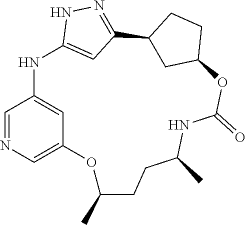 [SMILES: C[C@@H]1CC[C@H](C)NC(=O)O[C@@H]2CC[C@@H](C2)c2cc([nH]n2)Nc2cncc(c2)O1] | (1¹S,1³R,6R,9S,Z)-6,9-dimethyl- 2¹H-5,12-dioxa-3,10-diaza-4(3,5)- pyridina-2(3,5)-pyrazola-1(1,3)- cyclopentanacyclododecaphan-11- one |
| 198 | 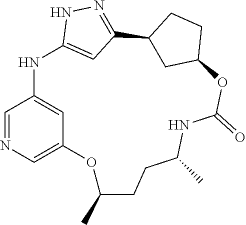 [SMILES: C[C@@H]1CC[C@@H](C)Oc2cncc(c2)Nc2cc(n[nH]2)[C@H]2CC[C@H](C2)OC(=O)N1] | (1¹S,1³R,6R,9R,Z)-6,9-dimethyl- 2¹H-5,12-dioxa-3,10-diaza-4(3,5)- pyridina-2(3,5)-pyrazola-1(1,3)- cyclopentanacyclododecaphan-11- one |
| 199 | 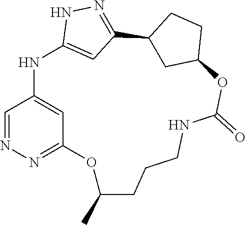 [SMILES: C[C@@H]1CCCNC(=O)O[C@@H]2CC[C@@H](C2)c2cc([nH]n2)Nc2cnnc(c2)O1] | (1¹S,1³R,6R,Z)-6-methyl-2¹H-5,12- dioxa-3,10-diaza-4(5,3)-pyridazina- 2(3,5)-pyrazola-1(1,3)- cyclopentanacyclododecaphan-11- one |
| 200 | 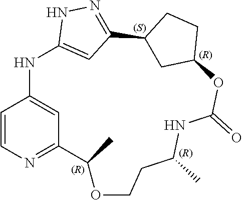 | (1¹S,1³R,5R,9R,Z)-5,9-dimethyl- 2¹H-6,12-dioxa-3,10-diaza-4(4,2)- pyridina-2(3,5)-pyrazola-1(1,3)- cyclopentanacyclododecaphan-11- one |
| 201 | 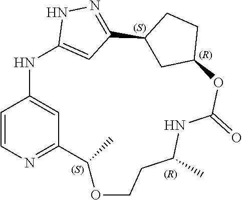 | (1¹S,1³R,5S,9R,Z)-5,9-dimethyl- 2¹H-6,12-dioxa-3,10-diaza-4(4,2)- pyridina-2(3,5)-pyrazola-1(1,3)- cyclopentanacyclododecaphan-11- one |
| 202 | 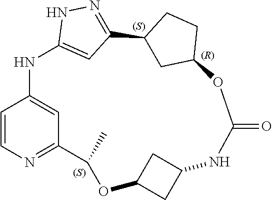 | (1¹S,1³R,7¹R,7³S,5S,Z)-5-methyl- 2¹H-6,10-dioxa-3,8-diaza-4(4,2)- pyridina-2(3,5)-pyrazola-1(1,3)- cyclopentana-7(1,3)- cyclobutanacyclodecaphan-9-one |
| 203 | 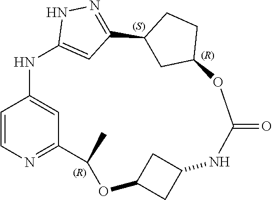 | (1¹S,1³R,7¹R,7³R,5R,Z)-5-methyl- 2¹H-6,10-dioxa-3,8-diaza-4(4,2)- pyridina-2(3,5)-pyrazola-1(1,3)- cyclopentana-7(1,3)- cyclobutanacyclodecaphan-9-one |
| 204 | 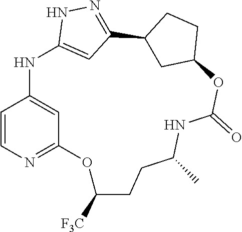 [SMILES: C[C@@H]1CC[C@@H](C(F)(F)F)Oc2cc(ccn2)Nc2cc(n[nH]2)[C@H]2CC[C@H](C2)OC(=O)N1] | (1¹S,1³R,6S,9R,Z)-9-methyl-6- (trifluoromethyl)-2¹H-5,12-dioxa- 3,10-diaza-4(4,2)-pyridina-2(3,5)- pyrazola-1(1,3)- cyclopentanacyclododecaphan-11- one |
| 205 | 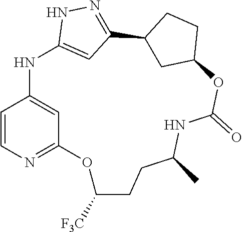 [SMILES: C[C@H]1CC[C@H](C(F)(F)F)Oc2cc(ccn2)Nc2cc(n[nH]2)[C@H]2CC[C@H](C2)OC(=O)N1] | (1¹S,1³R,6R,9S,Z)-9-methyl-6- (trifluoromethyl)-2¹H-5,12-dioxa- 3,10-diaza-4(4,2)-pyridina-2(3,5)- pyrazola-1(1,3)- cyclopentanacyclododecaphan-11- one |
| 206 | 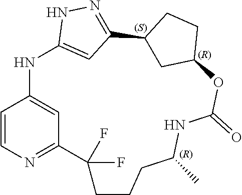 | (1¹S,1³R,9R,Z)-5,5-difluoro-9- methyl-2¹H-12-oxa-3,10-diaza- 4(4,2)-pyridina-2(3,5)-pyrazola- 1(1,3)- cyclopentanacyclododecaphan-11- one |
| 207 | 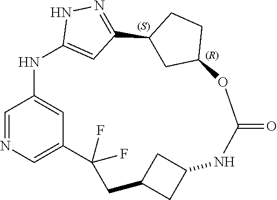 | (1¹S,1³R,7¹R,7³S,Z)-5,5-difluoro- 2¹H-10-oxa-3,8-diaza-4(3,5)- pyridina-2(3,5)-pyrazola-1(1,3)- cyclopentana-7(1,3)- cyclobutanacyclodecaphan-9-one |
| 208 | 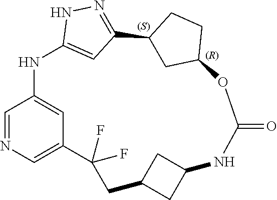 | (1¹S,1³R,7¹S,7³R,Z)-5,5-difluoro- 2¹H-10-oxa-3,8-diaza-4(3,5)- pyridina-2(3,5)-pyrazola-1(1,3)- cyclopentana-7(1,3)- cyclobutanacyclodecaphan-9-one |
| 209 | 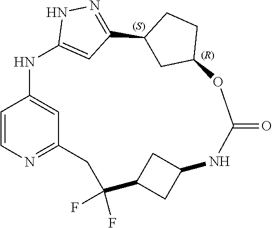 | (1¹S,1³R,7¹R,7³S,Z)-6,6-difluoro- 2¹H-10-oxa-3,8-diaza-4(4,2)- pyridina-2(3,5)-pyrazola-1(1,3)- cyclopentana-7(1,3)- cyclobutanacyclodecaphan-9-one |
| 210 | 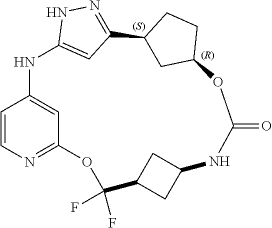 | (1¹S,1³R,7¹R,7³S,Z)-6,6-difluoro- 2¹H-5,10-dioxa-3,8-diaza-4(4,2)- pyridina-2(3,5)-pyrazola-1(1,3)- cyclopentana-7(1,3)- cyclobutanacyclodecaphan-9-one |
| 211 | 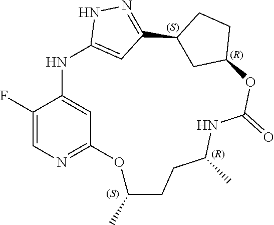 | (1¹S,1³R,6S,9R,Z)-4⁵-fluoro-6,9- dimethyl-2¹H-5,12-dioxa-3,10-diaza- 4(4,2)-pyridina-2(3,5)-pyrazola- 1(1,3)- cyclopentanacyclododecaphan-11- one |
| 212 | 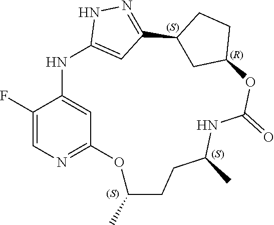 | (1¹S,1³R,6S,9S,Z)-4⁵-fluoro-6,9- dimethyl-2¹H-5,12-dioxa-3,10-diaza- 4(4,2)-pyridina-2(3,5)-pyrazola- 1(1,3)- cyclopentanacyclododecaphan-11- one |
| 213 | 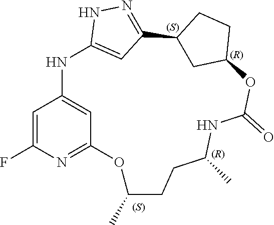 | (1¹S,1³R,6S,9R,Z)-4⁶-fluoro-6,9- dimethyl-2¹H-5,12-dioxa-3,10-diaza- 4(4,2)-pyridina-2(3,5)-pyrazola- 1(1,3)- cyclopentanacyclododecaphan-11- one |
| 214 | 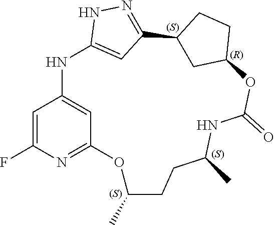 | (1¹S,1³R,6S,9S,Z)-4⁶-fluoro-6,9- dimethyl-2¹H-5,12-dioxa-3,10-diaza- 4(4,2)-pyridina-2(3,5)-pyrazola- 1(1,3)- cyclopentanacyclododecaphan-11- one |
| 215 | 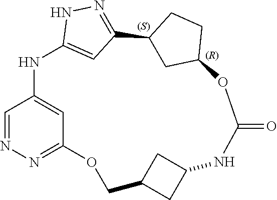 | (1¹S,1³R,7¹R,7³S,Z)-2¹H-5,10- dioxa-3,8-diaza-4(5,3)-pyridazina- 2(3,5)-pyrazola-1(1,3)- cyclopentana-7(1,3)- cyclobutanacyclodecaphan-9-one |
| 216 | 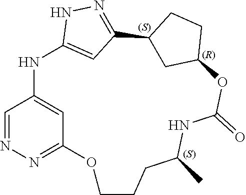 | (1¹S,1³R,9S,Z)-9-methyl-2¹H-5,12- dioxa-3,10-diaza-4(5,3)-pyridazina- 2(3,5)-pyrazola-1(1,3)- cyclopentanacyclododecaphan-11- one |
| 217 | 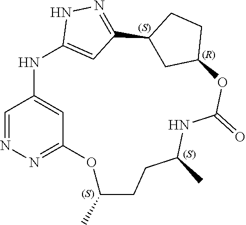 | (1¹S,1³R,6S,9S,Z)-6,9-dimethyl- 2¹H-5,12-dioxa-3,10-diaza-4(5,3)- pyridazina-2(3,5)-pyrazola-1(1,3)- cyclopentanacyclododecaphan-11- one |
| 218 | 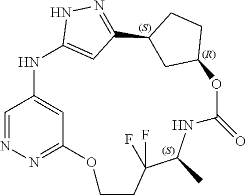 | (1¹S,1³R,9S,Z)-8,8-difluoro-9- methyl-2¹H-5,12-dioxa-3,10-diaza- 4(5,3)-pyridazina-2(3,5)-pyrazola- 1(1,3)- cyclopentanacyclododecaphan-11- one |
| 219 | 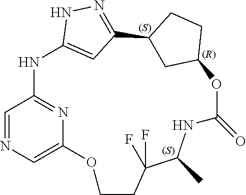 | (1¹S,1³R,9S,Z)-8,8-difluoro-9- methyl-2¹H-5,12-dioxa-3,10-diaza- 4(2,6)-pyrazina-2(3,5)-pyrazola- 1(1,3)- cyclopentanacyclododecaphan-11- one |
| 220 | 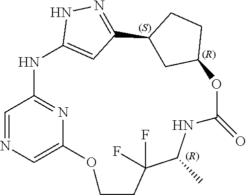 | (1¹S,1³R,9R,Z)-8,8-difluoro-9- methyl-2¹H-5,12-dioxa-3,10-diaza- 4(4,2)-pyridina-2(3,5)-pyrazola- 1(1,3)- cyclopentanacyclododecaphan-11- one |
| 221 | 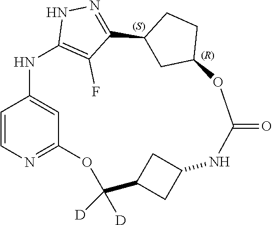 | (1¹S,1³R,7¹R,7³S,E)-24-fluoro-2¹H- 5,10-dioxa-3,8-diaza-4(4,2)- pyridina-2(3,5)-pyrazola-1(1,3)- cyclopentana-7(1,3)- cyclobutanacyclodecaphan-9-one- 6,6-d₂ |
| 222 | 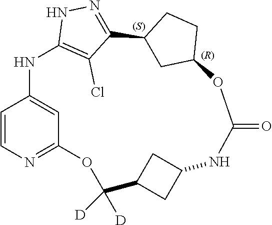 | (1¹S,1³R,7¹R,7³S,E)-24-chloro-2¹H- 5,10-dioxa-3,8-diaza-4(4,2)- pyridina-2(3,5)-pyrazola-1(1,3)- cyclopentana-7(1,3)- cyclobutanacyclodecaphan-9-one- 6,6-d₂ |
| 223 | 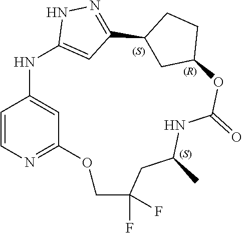 | (1¹S,1³R,9S,Z)-7,7-difluoro-9- methyl-2¹H-5,12-dioxa-3,10-diaza- 4(4,2)-pyridina-2(3,5)-pyrazola- 1(1,3)- cyclopentanacyclododecaphan-11- one |
| 224 | 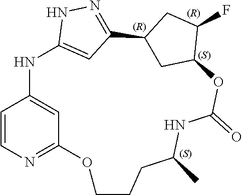 | (1¹R,1³S,1⁴R,9S,Z)-1⁴-fluoro-9- methyl-2¹H-5,12-dioxa-3,10-diaza- 4(4,2)-pyridina-2(3,5)-pyrazola- 1(1,3)- cyclopentanacyclododecaphan-11- one |
| 225 | 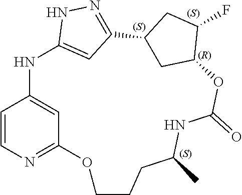 | (1¹S,1³R,1⁴S,9S,Z)-1⁴-fluoro-9- methyl-2¹H-5,12-dioxa-3,10-diaza- 4(4,2)-pyridina-2(3,5)-pyrazola- 1(1,3)- cyclopentanacyclododecaphan-11- one |
| 226 | 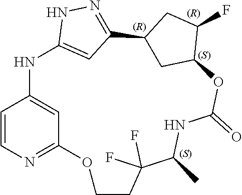 | (1¹R,1³S,1⁴R,9S,Z)-1⁴,8,8-trifluoro- 9-methyl-2¹H-5,12-dioxa-3,10- diaza-4(4,2)-pyridina-2(3,5)- pyrazola-1(1,3)- cyclopentanacyclododecaphan-11- one |
| 227 | 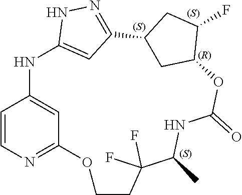 | (1¹S,1³R,1⁴S,9S,Z)-1⁴,8,8-trifluoro- 9-methyl-2¹H-5,12-dioxa-3,10- diaza-4(4,2)-pyridina-2(3,5)- pyrazola-1(1,3)- cyclopentanacyclododecaphan-11- one |
| 228 | 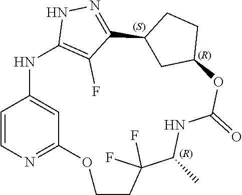 | (1¹S,1³R,9R,E)-2⁴,8,8-trifluoro-9- methyl-2¹H-5,12-dioxa-3,10-diaza- 4(4,2)-pyridina-2(3,5)-pyrazola- 1(1,3)- cyclopentanacyclododecaphan-11- one |
| 229 | 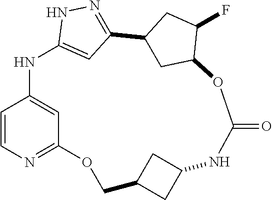 | (1¹R,1³S,1⁴R,7¹S,7³R,Z)-1⁴-fluoro- 2¹H-5,10-dioxa-3,8-diaza-4(4,2)- pyridina-2(3,5)-pyrazola-1(1,3)- cyclopentana-7(1,3)- cyclobutanacyclodecaphan-9-one |
| 230 | 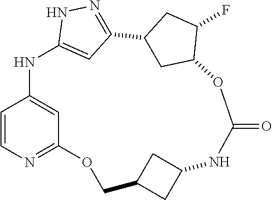 | (1¹S,1³R,1⁴S,7¹R,7³S,Z)-1⁴-fluoro- 2¹H-5,10-dioxa-3,8-diaza-4(4,2)- pyridina-2(3,5)-pyrazola-1(1,3)- cyclopentana-7(1,3)- cyclobutanacyclodecaphan-9-one |
| 231 | 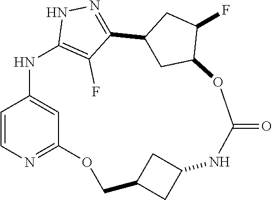 | (1¹R,1³S,1⁴R,7¹S,7³R,E)-1⁴,2⁴- difluoro-2¹H-5,10-dioxa-3,8-diaza- 4(4,2)-pyridina-2(3,5)-pyrazola- 1(1,3)-cyclopentana-7(1,3)- cyclobutanacyclodecaphan-9-one |
| 232 | 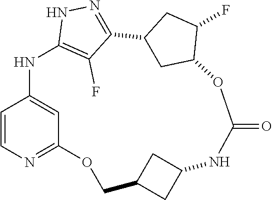 | (1¹S,1³R,1⁴S,7¹R,7³S,E)-1⁴,2⁴- difluoro-2¹H-5,10-dioxa-3,8-diaza- 4(4,2)-pyridina-2(3,5)-pyrazola- 1(1,3)-cyclopentana-7(1,3)- cyclobutanacyclodecaphan-9-one |
| 233 | 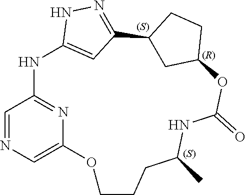 | (1¹S,1³R,9S,Z)-9-methyl-2¹H-5,12- dioxa-3,10-diaza-4(2,6)-pyrazina- 2(3,5)-pyrazola-1(1,3)- cyclopentanacyclododecaphan-11- one |
| 234 | 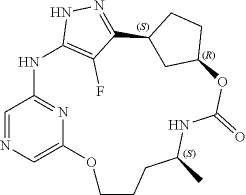 | (1¹S,1³R,9S,E)-2⁴-fluoro-9-methyl- 2¹H-5,12-dioxa-3,10-diaza-4(2,6)- pyrazina-2(3,5)-pyrazola-1(1,3)- cyclopentanacyclododecaphan-11- one |
| 235 | 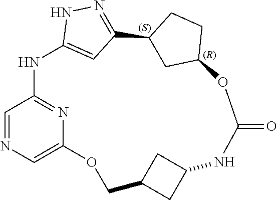 | (1¹S,1³R,7¹R,7³S,Z)-2¹H-5,10- dioxa-3,8-diaza-4(2,6)-pyrazina- 2(3,5)-pyrazola-1(1,3)- cyclopentana-7(1,3)- cyclobutanacyclodecaphan-9-one |
| 236 | 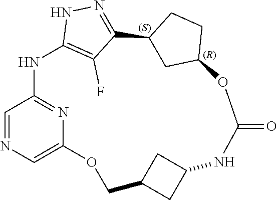 | (1¹S,1³R,7¹R,7³S,E)-2⁴-fluoro-2¹H- 5,10-dioxa-3,8-diaza-4(2,6)- pyrazina-2(3,5)-pyrazola-1(1,3)- cyclopentana-7(1,3)- cyclobutanacyclodecaphan-9-one |
| 237 | 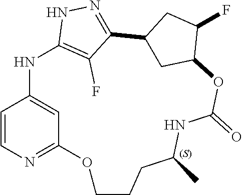 | (1¹R,1³S,1⁴R,9S,E)-1⁴,2⁴-difluoro-9- methyl-2¹H-5,12-dioxa-3,10-diaza- 4(4,2)-pyridina-2(3,5)-pyrazola- 1(1,3)- cyclopentanacyclododecaphan-11- one |
| 238 | 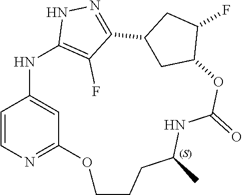 | (1¹S,1³R,1⁴S,9S,E)-1⁴,2⁴-difluoro-9- methyl-2¹H-5,12-dioxa-3,10-diaza- 4(4,2)-pyridina-2(3,5)-pyrazola- 1(1,3)- cyclopentanacyclododecaphan-11- one |
| 239 | 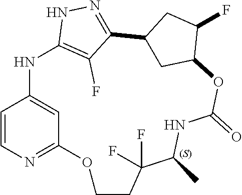 | (1¹R,1³S,1⁴R,9S,E)-1⁴,2⁴,8,8- tetrafluoro-9-methyl-2¹H-5,12-dioxa- 3,10-diaza-4(4,2)-pyridina-2(3,5)- pyrazola-1(1,3)- cyclopentanacyclododecaphan-11- one |
| 240 | 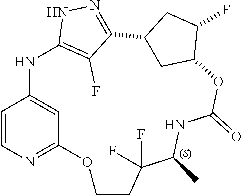 | (1¹S,1³R,1⁴S,9S,E)-1⁴,2⁴,8,8- tetrafluoro-9-methyl-2¹H-5,12-dioxa- 3,10-diaza-4(4,2)-pyridina-2(3,5)- pyrazola-1(1,3)- cyclopentanacyclododecaphan-11- one |
| 241 | 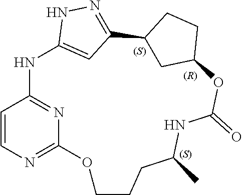 | (1¹S,1³R,9S,Z)-9-methyl-2¹H-5,12- dioxa-3,10-diaza-4(4,2)-pyrimidina- 2(3,5)-pyrazola-1(1,3)- cyclopentanacyclododecaphan-11- one |
| 242 | 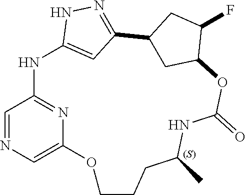 | (1¹R,1³S,1⁴R,9S,Z)-1⁴-fluoro-9- methyl-2¹H-5,12-dioxa-3,10-diaza- 4(2,6)-pyrazina-2(3,5)-pyrazola- 1(1,3)- cyclopentanacyclododecaphan-11- one |
| 243 | 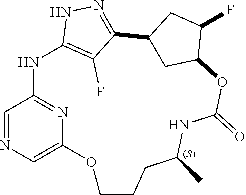 | (1¹R,1³S,1⁴R,9S,E)-1⁴,2⁴-difluoro-9- methyl-2¹H-5,12-dioxa-3,10-diaza- 4(2,6)-pyrazina-2(3,5)-pyrazola- 1(1,3)- cyclopentanacyclododecaphan-11- one |
| 244 | 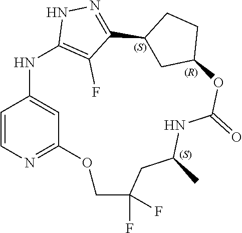 | (1¹S,1³R,9S,E)-2⁴,7,7-trifluoro-9- methyl-2¹H-5,12-dioxa-3,10-diaza- 4(4,2)-pyridina-2(3,5)-pyrazola- 1(1,3)- cyclopentanacyclododecaphan-11- one |
| 245 | 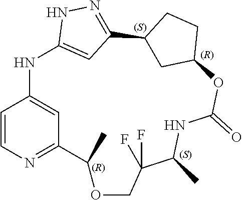 | (1¹S,1³R,5R,9S,Z)-8,8-difluoro-5,9- dimethyl-2¹H-6,12-dioxa-3,10-diaza- 4(4,2)-pyridina-2(3,5)-pyrazola- 1(1,3)- cyclopentanacyclododecaphan-11- one |
| 246 | 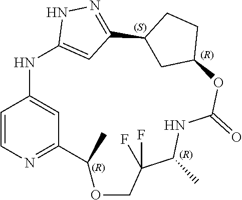 | (1¹S,1³R,5R,9R,Z)-8,8-difluoro-5,9- dimethyl-2¹H-6,12-dioxa-3,10-diaza- 4(4,2)-pyridina-2(3,5)-pyrazola- 1(1,3)- cyclopentanacyclododecaphan-11- one |
| 247 | 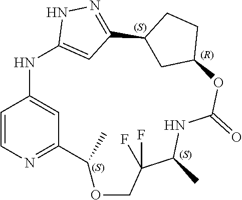 | (1¹S,1³R,5S,9S,Z)-8,8-difluoro-5,9- dimethyl-2¹H-6,12-dioxa-3,10-diaza- 4(4,2)-pyridina-2(3,5)-pyrazola- 1(1,3)- cyclopentanacyclododecaphan-11- one |
| 248 | 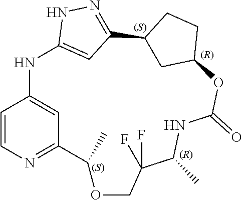 | (1¹S,1³R,5S,9R,Z)-8,8-difluoro-5,9- dimethyl-2¹H-6,12-dioxa-3,10-diaza- 4(4,2)-pyridina-2(3,5)-pyrazola- 1(1,3)- cyclopentanacyclododecaphan-11- one |
| 249 | 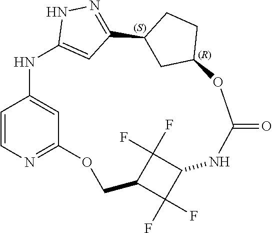 | (1¹S,1³R,7¹R,7³S,Z)-7²,7²,7⁴,7⁴- tetrafluoro-2¹H-5,10-dioxa-3,8- diaza-4(4,2)-pyridina-2(3,5)- pyrazola-1(1,3)-cyclopentana- 7(1,3)-cyclobutanacyclodecaphan- 9-one |
| 250 | 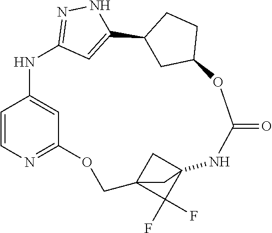 | (1¹S,1³R,Z)-7²,7²-difluoro-2¹H-5,10- dioxa-3,8-diaza-4(4,2)-pyridina- 2(5,3)-pyrazola-7(1,3)- bicyclo[1.1.1]pentana-1(1,3)- cyclopentanacyclodecaphan-9-one |
| 251 | 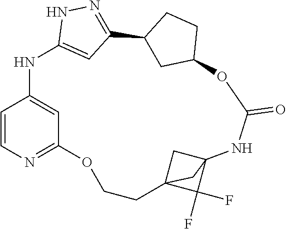 [SMILES: O=C1NC23CC(CCOc4cc(ccn4)Nc4cc(n[nH]4)[C@H]4CC[C@H](C4)O1)(C2)C3(F)F] | (1¹S,1³R,Z)-8²,8²-difluoro-2¹H-5,11- dioxa-3,9-diaza-4(4,2)-pyridina- 2(3,5)-pyrazola-8(1,3)- bicyclo[1.1.1]pentana-1(1,3)- cyclopentanacycloundecaphan-10- one |
| 252 | 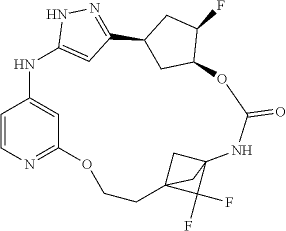 [SMILES: O=C1NC23CC(CCOc4cc(ccn4)Nc4cc(n[nH]4)[C@H]4C[C@@H](F)[C@H](C4)O1)(C2)C3(F)F] | (1¹R,1³S,1⁴R,Z)-1⁴,8²,8²-trifluoro- 2¹H-5,11-dioxa-3,9-diaza-4(4,2)- pyridina-2(3,5)-pyrazola-8(1,3)- bicyclo[1.1.1]pentana-1(1,3)- cyclopentanacycloundecaphan-10- one |
| 253 | 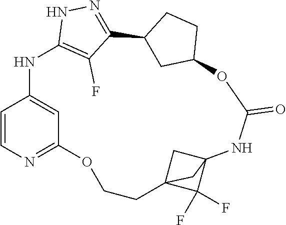 [SMILES: O=C1NC23CC(CCOc4cc(ccn4)Nc4[nH]nc(c4F)[C@H]4CC[C@H](C4)O1)(C2)C3(F)F] | (1¹S,1³R,E)-2⁴,8²,8²-trifluoro-2¹H- 5,11-dioxa-3,9-diaza-4(4,2)- pyridina-2(3,5)-pyrazola-8(1,3)- bicyclo[1.1.1]pentana-1(1,3)- cyclopentanacycloundecaphan-10- one |
| 254 | 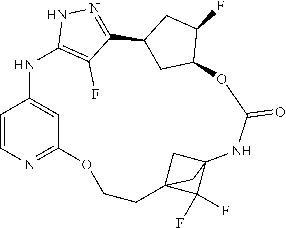 [SMILES: O=C1NC23CC(CCOc4cc(ccn4)Nc4[nH]nc(c4F)[C@H]4C[C@@H](F)[C@H](C4)O1)(C2)C3(F)F] | (1¹R,1³S,1⁴R,E)-1⁴,2⁴,8²,8²- tetrafluoro-2¹H-5,11-dioxa-3,9- diaza-4(4,2)-pyridina-2(3,5)- pyrazola-8(1,3)- bicyclo[1.1.1]pentana-1(1,3)- cyclopentanacycloundecaphan-10- one |
| 255 | 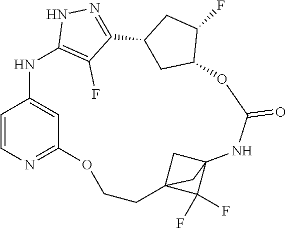 [SMILES: O=C1NC23CC(CCOc4cc(ccn4)Nc4[nH]nc(c4F)[C@@H]4C[C@H](F)[C@@H](C4)O1)(C2)C3(F)F] | (1¹S,1³R,1⁴S,E)-1⁴,2⁴,8²,8²- tetrafluoro-2¹H-5,11-dioxa-3,9- diaza-4(4,2)-pyridina-2(3,5)- pyrazola-8(1,3)- bicyclo[1.1.1]pentana-1(1,3)- cyclopentanacycloundecaphan-10- one |
| 256 | 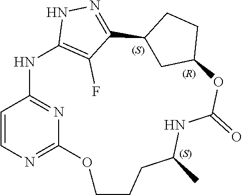 | (1¹S,1³R,9S,E)-2⁴-fluoro-9-methyl- 2¹H-5,12-dioxa-3,10-diaza-4(4,2)- pyrimidina-2(3,5)-pyrazola-1(1,3)- cyclopentanacyclododecaphan-11- one |
| 257 | 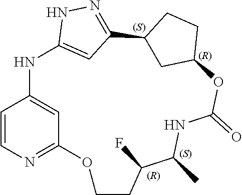 | (1¹S,1³R,8R,9S,Z)-8-fluoro-9- methyl-2¹H-5,12-dioxa-3,10-diaza- 4(4,2)-pyridina-2(3,5)-pyrazola- 1(1,3)- cyclopentanacyclododecaphan-11- one |
| 258 | 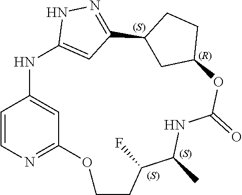 | (1¹S,1³R,8S,9S,Z)-8-fluoro-9- methyl-2¹H-5,12-dioxa-3,10-diaza- 4(4,2)-pyridina-2(3,5)-pyrazola- 1(1,3)- cyclopentanacyclododecaphan-11- one |

### Pharmaceutical Compositions

Pharmaceutical compositions of the present disclosure comprise at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, with at least one pharmaceutically acceptable carrier. These formulations include those suitable for oral, rectal, topical, buccal and parenteral (e.g., subcutaneous, intramuscular, intradermal, or intravenous) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least one compound of the present disclosure, which may be optionally combined with one or more accessory ingredients. Compressed tablets may be prepared by compressing, in a suitable machine, at least one compound of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one compound of the present disclosure in a flavored base, usually sucrose and acacia or tragacanth, and pastilles comprising the at least one compound in an inert base such as gelatin and glycerin or sucrose and acacia.

Formulations of the present disclosure suitable for parenteral administration comprise sterile aqueous preparations of at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, which are approximately isotonic with the blood of the intended recipient. These preparations are administered intravenously, although administration may also be affected by means of subcutaneous, intramuscular, or intradermal injection. Such preparations may conveniently be prepared by admixing at least one compound described herein with water and rendering the resulting solution sterile and isotonic with the blood. Injectable compositions according to the present disclosure may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and the following table (Table 3) for Equivalent Surface Area Dosage Factors).

TABLE 3
Equivalent Surface Area Dosage Factors.

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject.

The dosage may be determined by a physician and adjusted, as necessary, to suit observed effects of the treatment.

### Methods of Treatment

In some embodiments, at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, is administered to treat cancer in a subject in need thereof. In some embodiments, the cancer is chosen from breast cancer, ovarian cancer, uterine cancer, endometrial cancer, cervical cancer, bladder cancer, prostate cancer, lung cancer, stomach cancer, esophageal cancer, colorectal cancer, small bowel cancer, pancreatic cancer, liver cancer, kidney cancer, head and neck cancer, skin cancer, bone cancer, thyroid cancer, peritoneal cancer, and brain cancer. In some embodiments, the cancer is breast cancer. In some embodiments, the cancer is ovarian cancer. In some embodiments, the cancer is uterine cancer. In some embodiments, the cancer is endometrial cancer. In some embodiments, the cancer is cervical cancer. In some embodiments, the cancer is bladder cancer. In some embodiments, the cancer is prostate cancer. In some embodiments, the cancer is lung cancer. In some embodiments, the cancer is stomach cancer. In some embodiments, the cancer is esophageal cancer. In some embodiments, the cancer is colorectal cancer. In some embodiments, the cancer is small bowel cancer. In some embodiments, the cancer is pancreatic cancer. In some embodiments, the cancer is liver cancer. In some embodiments, the cancer is kidney cancer. In some embodiments, the cancer is head and neck cancer. In some embodiments, the cancer is skin cancer. In some embodiments, the cancer is bone cancer. In some embodiments, the cancer is thyroid cancer. In some embodiments, the cancer is peritoneal cancer. In some embodiments, the cancer is brain cancer.

In some embodiments, a compound of Formula (I), Formula (II), Formula (III), Formula (IV), or Formula (V), or tautomers, stereoisomers, pharmaceutically acceptable salts, hydrates, or deuterated derivatives of any of the foregoing, is administered as a pharmaceutical composition.

In some embodiments, the subject has been previously treated with an anti-cancer agent. In some embodiments, the anti-cancer agent is enzalutamide, apalutamide, bicalutamide, darolutamide, flutamide, abiratarone, or a combination of any of the foregoing. In some embodiments, the anti-cancer agent is enzalutamide.

In some embodiments, provided herein is a use of at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, for treating cancer. In some embodiments, the cancer is selected from breast cancer, ovarian cancer, uterine cancer, endometrial cancer, cervical cancer, bladder cancer, prostate cancer, lung cancer, stomach cancer, esophageal cancer, colorectal cancer, small bowel cancer, pancreatic cancer, liver cancer, kidney cancer, head and neck cancer, skin cancer, bone cancer, thyroid cancer, peritoneal cancer, and brain cancer. In some embodiments, the cancer is breast cancer. In some embodiments, the cancer is ovarian cancer. In some embodiments, the cancer is uterine cancer. In some embodiments, the cancer is endometrial cancer. In some embodiments, the cancer is cervical cancer. In some embodiments, the cancer is bladder cancer. In some embodiments, the cancer is prostate cancer. In some embodiments, the cancer is lung cancer. In some embodiments, the cancer is stomach cancer. In some embodiments, the cancer is esophageal cancer. In some embodiments, the cancer is colorectal cancer. In some embodiments, the cancer is small bowel cancer. In some embodiments, the cancer is pancreatic cancer. In some embodiments, the cancer is liver cancer. In some embodiments, the cancer is kidney cancer. In some embodiments, the cancer is head and neck cancer. In some embodiments, the cancer is skin cancer. In some embodiments, the cancer is bone cancer. In some embodiments, the cancer is thyroid cancer. In some embodiments, the cancer is peritoneal cancer. In some embodiments, the cancer is brain cancer.

In some embodiments, provided herein is a use of at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, in the preparation of a medicament. In some embodiments, the medicament is for the treatment of cancer. In some embodiments, the cancer is selected from breast cancer, ovarian cancer, uterine cancer, endometrial cancer, cervical cancer, bladder cancer, prostate cancer, lung cancer, stomach cancer, esophageal cancer, colorectal cancer, small bowel cancer, pancreatic cancer, liver cancer, kidney cancer, head and neck cancer, skin cancer, bone cancer, thyroid cancer, peritoneal cancer, and brain cancer. In some embodiments, the cancer is breast cancer. In some embodiments, the cancer is ovarian cancer. In some embodiments, the cancer is uterine cancer. In some embodiments, the cancer is endometrial cancer. In some embodiments, the cancer is cervical cancer. In some embodiments, the cancer is bladder cancer. In some embodiments, the cancer is prostate cancer. In some embodiments, the cancer is lung cancer. In some embodiments, the cancer is stomach cancer. In some embodiments, the cancer is esophageal cancer. In some embodiments, the cancer is colorectal cancer. In some embodiments, the cancer is small bowel cancer. In some embodiments, the cancer is pancreatic cancer. In some embodiments, the cancer is liver cancer. In some embodiments, the cancer is kidney cancer. In some embodiments, the cancer is head and neck cancer. In some embodiments, the cancer is skin cancer. In some embodiments, the cancer is bone cancer. In some embodiments, the cancer is thyroid cancer. In some embodiments, the cancer is peritoneal cancer. In some embodiments, the cancer is brain cancer.

In some embodiments, provided herein is a method of treating a disease or condition modulated at least in part by CK2 in a subject comprising administering to the subject in need thereof with at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing.

In some embodiments, at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, may be administered in combination with another therapeutic agent. The other therapeutic agent can provide additive or synergistic value relative to the administration of a compound of the present disclosure alone. The therapeutic agent can be selected from, for example, hormones and hormonal analogues; signal transduction pathway inhibitors; topoisomerase I inhibitors; topoisomerase II inhibitors; antimetabolite neoplastic agents; antibiotic neoplastic agents; alkylating agents; anti-microtubule agents; platinum coordination complexes; aromatase inhibitors; and anti-mitotic agents.

In some embodiments, the therapeutic agent may be a hormone or hormonal analogue. In some embodiments, the therapeutic agent may be a signal transduction pathway inhibitor. In some embodiments, the therapeutic agent may be a topoisomerase I inhibitor. In some embodiments, the therapeutic agent may be a topoisomerase II inhibitor. In some embodiments, the therapeutic agent may be an antimetabolite neoplastic agent. In some embodiments, the therapeutic agent may be an antibiotic neoplastic agent. In some embodiments, the therapeutic agent may be an alkylating agent. In some embodiments, the therapeutic agent may be an anti-microtubule agent. In some embodiments, the therapeutic agent may be a platinum coordination complex. In some embodiments, the therapeutic agent may be an aromatase inhibitor. In some embodiments, the therapeutic agent may be an anti-mitotic agent.

In some embodiments, the aromatase inhibitor may be selected from anastrazole, letrozole, vorozole, fadrozole, exemestane, and formestane. In some embodiments, the aromatase inhibitor is anastrazole. In some embodiments, the aromatase inhibitor may be letrozole. In some embodiments, the aromatase inhibitor may be vorozole. In some embodiments, the aromatase inhibitor may be fadrozole. In some embodiments, the aromatase inhibitor may be exemestane. In some embodiments, the aromatase inhibitor may be formestane.

In some embodiments, the anti-mitotic agent may be selected from paclitaxel, docetaxel, and Abraxane. In some embodiments, the anti-mitotic agent may be paclitaxel. In some embodiments, the anti-mitotic agent may be docetaxel. In some embodiments, the anti-mitotic agent may be Abraxane.

In some embodiments, at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, may be administered in combination with a hormone or hormonal analog. In some embodiments, at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, may be administered in combination with a signal transduction pathway inhibitor. In some embodiments, at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, may be administered in combination with an antimetabolite neoplastic agent. In some embodiments, at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, may be administered in combination with a topoisomerase I inhibitor. In some embodiments, at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, may be administered in combination with a topoisomerase II inhibitor. In some embodiments, at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing, may be administered in combination with an aromatase inhibitor.

### EXAMPLES

The examples and preparations provided below further illustrate and exemplify the compounds as disclosed herein and methods of preparing such compounds. It is to be understood that the scope of the present disclosure is not limited in any way by the scope of the following examples and preparations.

### General Synthetic Schemes

Compounds of at least one entity chosen from the compounds of Formula (I), Formula (II), Formula (III), Formula (IV), and Formula (V), and tautomers, stereoisomers, pharmaceutically acceptable salts, hydrates, and deuterated derivatives of any of the foregoing can be prepared according to the following schemes. The following schemes represent the general methods used in preparing these compounds. However, the synthesis of these compounds is not limited to these representative methods, as they can also be prepared by various other methods those skilled in the art of synthetic chemistry, for example, in a stepwise or modular fashion.

As described in Scheme 1, the displacement of fluorine on heteroaromatic ring 1-A by alkoxide 1-B yields an ether 1-C that undergoes palladium-catalyzed Buchwald amination with intermediate 1-D to form intermediate 1-E. Protection of the NH group of 1 E with Boc₂O anhydride affords 1-F, which is subjected to TBAF promoted deprotection that removes the silyl ether group. Thus, the key intermediate 1-G bearing cyclopentan-3-ol functionality is free. The reaction of the hydroxyl group in 1-G with 4-nitrophenyl chloroformate results in the formation of the activated carbonate 1-H. The deprotection of the Boc group in 1-H, followed by the intramolecular cyclization provides carbamate 1-J. The removal of the tert-butyl group on the pyrazolyl ring in 1-J generates the desired macrocyclic compounds.

As described in Scheme 2, Mitsunobu reaction of 2-A with hydroxyl group in 1-B in the presence of cyanomethylenetributylphosphorane (CMBP) affords the aryl ether 2-B. The reaction of 2-B with 1-D under Buchwald amination conditions and followed by similar steps as described in Scheme 1 generate the desired macrocyclic molecules.

As described in Scheme 3, Sonogashira coupling of 3-A with alkyne 3-B forms the coupling product 3-C, which undergoes Buchwald amination to provide intermediate 3-D. Hydrogenation of 3-D gives 3-E which can be converted to the desired macrocyclic target following similar transformations described in Scheme 1 and Scheme 2.

### Abbreviations

The following abbreviations have the meanings set forth below:
Ac₂O: Acetic anhydride
BH₃THF: Borane-tetrahydrofuran
Boc: tert-Butyloxycarbonyl protecting group
Cs₂CO₃: Cesium carbonate
CMBP: cyanomethylenetributylphosphorane
m-CPBA: meta-chloroperoxybenzoic acid
(COCl)₂: Oxalyl chloride
CuI: Copper iodide
DAST: Diethylaminosulfur trifluoride
DCM: Dichloromethane
DIAD: Diisopropyl azodicarboxylate
DIBAL-H: Diisobutylaluminium hydride
DIEA: N,N-Diisopropylethylamine
DMAP: 4-Dimethylaminopyridine
DME: Dimethoxyethane
DMF: Dimethylformamide
DMSO: Dimethyl sulfoxide
DIPEA: N, N-Diisopropylethylamine
EA: Ethyl acetate
EtOAc: Ethyl acetate
FA: Formic acid
HATU: 1-[Bis(dimethylamino)methylene]-1H-1,2,3-triazolo[4,5-b]pyridinium 3-oxide hexafluorophosphate
KCN: Potassium cyanide
LiAlH₄: Lithium aluminium hydride
LiBH₄: Lithium borohydride
LiCl: Lithium chloride
LiOH: Lithium hydroxide
MeOH: Methanol
MeMgBr: Methylmagnesium bromide
Ms₂O: Methanesulfonic anhydride
NaBH₄: Sodium borohydride
NH₄F: Ammonium fluoride
OTBS: tert-Butyldimethylsilyl ether
PA: Petroleum ether
POBr₃: Phosphoryl bromide
Pd₂(dba)₃: Tris(dibenzylideneacetone)dipalladium(0)
Pd(PPh₃)₂Cl₂: Bis(triphenylphosphine)palladium(II) dichloride
Pd(PPh₃)₄: Tetrakis(triphenylphosphine)palladium(0)
PPh₃: Triphenylphosphine
TBAF: Tetra-n-butylammonium fluoride
t-BuOK: Potassium tert-butoxide
TEA: Triethylamine
TFA: Trifluoroacetic acid
THF: Tetrahydrofuran
Pd(OH)₂/C: Palladium hydroxide on carbon
Pd/C: Palladium on carbon
Pt/C: Platinum on carbon
Py: Pyridine
Xantphos: (9,9-Dimethyl-9H-xanthene-4,5-diyl)bis(diphenylphosphane)

Compounds described in the experimental were prepared from commercially available material. Purity of all final compounds were analyzed by HPLC with detection at 214 nM and 254 nM wavelength. All final compounds showed purity greater than 95%. All final compounds were characterized by LC/MS and H-NMR. The following are representative examples demonstrating how the claimed molecules can be made, however, a person of skill in the art would understand that the compounds could be prepared by other synthetic methods.

### PREPARATION OF EXAMPLE COMPOUNDS

### Example 1: Synthesis of (1¹S,1³R,Z)-4⁶-(difluoromethyl)-2¹H-5,12-dioxa-3,10-diaza-4(4,2)-pyridina-2(5,3)-pyrazola-1 (1,3)-cyclopentanacyclododecaphan-11-one (Compound 16)

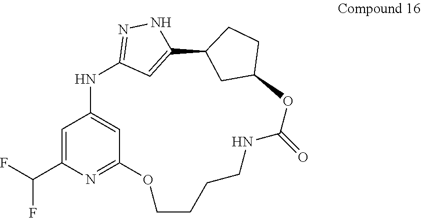 [SMILES: O=C1NCCCCOc2cc(cc(C(F)F)n2)Nc2cc([nH]n2)[C@H]2CC[C@H](C2)O1]

### Step 1: Preparation of 4-bromo-2-(difluoromethyl)pyridine 1-oxide

To a solution of 4-bromo-2-(difluoromethyl)pyridine (5 g, 24.04 mmol, 1 eq.) in DCM (180 mL), m-CPBA (29.28 g, 144.23 mmol, 85% purity, 6 eq.) was added, and the mixture was stirred at 25° C. for 12 hours under N₂ atmosphere. After the reaction was completed, the reaction solution was poured into aqueous sodium sulfite (180 mL) and extracted with EtOAc (200 mL×3). The combined organic phase was washed with brine (50 mL), dried over Na₂SO₄, filtered, and the filtrate was evaporated under reduced pressure to give the crude, which was purified by column chromatography. The desired 4-bromo-2-(difluoromethyl)pyridine 1-oxide (4.84 g, 45.84% yield) was obtained as a white solid. LCMS: 223.9, 225.9 [M+H]⁺.

### Step 2: Preparation of 4-bromo-6-(difluoromethyl)pyridin-2-ol

A solution of 4-bromo-2-(difluoromethyl)pyridine 1-oxide (1.77 g, 7.90 mmol, 1 eq.) in Ac₂O (8 mL) was heated and stirred at 150° C. for 12 hours under N₂ atmosphere. The reaction mixture was cooled to room temperature and evaporated under reduced pressure. The resulting crude was purified by column chromatography to give 4-bromo-6-(difluoromethyl)pyridin-2-ol (263 mg, 13.7% yield) as an orange oil. LCMS: 224.0, 226.0 [M+H]⁺; ¹H NMR (400 MHz, CDCl₃) δ 8.82 (s, 1H), 7.75 (s, 1H), 7.46 (s, 1H), 6.73-6.31 (m, 1H) ppm.

### Step 3: Preparation of tert-butyl (4-((4-bromo-6-(difluoromethyl)pyridin-2-yl)oxy)butyl)carbamate

To a solution of 4-bromo-6-(difluoromethyl)pyridin-2-ol (260 mg, 1.16 mmol, 1 eq.) and tert-butyl (4-hydroxybutyl)carbamate (263.60 mg, 1.39 mmol, 1.2 eq.) in toluene (3 mL) was added 2-(tributyl-phosphanylidene)acetonitrile (CMBP, 560.28 mg, 2.32 mmol, 2 eq.), and the mixture was stirred at 80° C. for 12 hours under N₂ atmosphere. The mixture was then cooled to room temperature, diluted with water (30 mL) and extracted with EtOAc (30 mL×3). The combined organic phase was washed with brine (50 mL), dried over Na₂SO₄, filtered, and the filtrate was evaporated under reduced pressure. The resulting residue was purified by Prep-TLC with PE:EA=3:1 to give tert-butyl (4-((4-bromo-6-(difluoromethyl)pyridin-2-yl)oxy)butyl)carbamate (196 mg, 40.59% yield) as an orange solid. LCMS: 339.0, 341.0 [M−56]+; ¹H NMR (400 MHz, CDCl₃) δ 7.34 (s, 1H), 7.02 (s, 1H), 6.65-6.27 (m, 1H), 4.69-4.50 (m, 1H), 4.33 (t, J=6.38 Hz, 2H), 3.31 (br t, J=6.57 Hz, 2H), 3.19 (br d, J=6.25 Hz, 2H), 1.86-1.82 (m, 2H), 1.64 (br d, J=7.63 Hz, 2H), 1.45 (s, 9H) ppm.

### Step 4: Preparation of tert-butyl (4-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)oxy)butyl)carbamate

The mixture of tert-butyl (4-((4-bromo-6-(difluoromethyl)pyridin-2-yl)oxy)butyl)carbamate (180 mg, 0.455 mmol, 1 eq.), 1-(tert-butyl)-3-((1S,3R)-3-((tert-butyldimethylsilyl)oxy)cyclopentyl)-1H-pyrazol-5-amine (153.74 mg, 0.455 mmol, 1 eq.), Pd₂(dba)₃ (41.70 mg, 0.0455 mmol, 0.1 eq), Xantphos (39.53 mg, 0.0683 mmol, 0.15 eq) and Cs₂CO₃ (445.16 mg, 1.37 mmol, 3 eq) in dioxane (2 mL) was stirred at 80° C. for 6 hours under N₂ atmosphere. The reaction solution was then cooled to room temperature, diluted with water (20 mL) and extracted with EtOAc (20 mL×3). The combined organic phase was washed with brine (20 mL), dried over Na₂SO₄, filtered, and the filtrate was evaporated under reduced pressure. The resulting residue was purified by Prep-TLC (PE:EA=3:1) to give tert-butyl (4-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)oxy)butyl)carbamate (251 mg, 80.32% yield) as an orange solid. LCMS: 652.4 [M+H]⁺.

Step 5: Preparation of tert-butyl (2-(4-((tert-butoxycarbonyl)amino)butoxy)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (4-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)oxy)butyl)carbamate (240 mg, 0.368 mmol, 1 eq.), DMAP (44.98 mg, 0.368 mmol, 1 eq.) and TEA (111.76 mg, 1.10 mmol, 153.73 μL, 3 eq.) in THF (8 mL) was slowly added Boc₂O (160.70 mg, 0.736 mmol, 169.16 μL, 2 eq.) at 0° C., and the mixture was warmed to 25° C. and kept stirring for 2 hours under N₂ atmosphere. The reaction mixture was then diluted with water (8 mL) and extracted with EtOAc (8 mL×3). The combined organic phase was washed with brine (10 mL), dried over Na₂SO₄, filtered, and the filtrate was evaporated under reduced pressure. The resulting residue was purified by Prep-TLC (PE:EA=5:1) to give tert-butyl (2-(4-((tert-butoxycarbonyl)amino)butoxy)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate (260 mg, 89.22% yield) as a colorless oil. LCMS: 752.3 [M+H]⁺.

Step 6: Preparation of tert-butyl (2-(4-((tert-butoxycarbonyl)amino)butoxy)-6-(difluoromethyl)pyridin-4-yl)(1-(tert-butyl)-3-((1S,3R)-3-hydroxycyclopentyl)-1H-pyrazol-5-yl)carbamate

The solution of tert-butyl (2-(4-((tert-butoxycarbonyl)amino)butoxy)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate (240 mg, 0.319 mmol, 1 eq.) and TBAF (1 M, 319.14 μL, 1 eq.) in THF (5 mL) was stirred at 25° C. for 12 hours under N₂ atmosphere. After the reaction was completed, the mixture was poured into H₂O (20 mL) and extracted with EtOAc (20 mL×3). The combined organic layers were washed with brine (10 mL), dried over Na₂SO₄, filtered, and the filtrate was evaporated under reduced pressure. The resulting residue was purified with column chromatography to give tert-butyl (2-(4-((tert-butoxycarbonyl)amino)butoxy)-6-(difluoromethyl)pyridin-4-yl)(1-(tert-butyl)-3-((1S,3R)-3-hydroxycyclopentyl)-1H-pyrazol-5-yl)carbamate (170 mg, 83.52% yield) as a colorless gum. LCMS: 638.3 [M+H]⁺.

Step 7: Preparation of tert-butyl (2-(4-((tert-butoxycarbonyl)amino)butoxy)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(4-((tert-butoxycarbonyl)amino)butoxy)-6-(difluoromethyl)pyridin-4-yl)(1-(tert-butyl)-3-((1S,3R)-3-hydroxycyclopentyl)-1H-pyrazol-5-yl)carbamate (160 mg, 0.251 mmol, 1 eq.) in THF (8 mL) was added pyridine (99.22 mg, 1.25 mmol, 101.25 μL, 5 eq.) and DMAP (15.32 mg, 0.125 mmol, 0.5 eq.) at 0° C. under N₂ atmosphere, followed by slow addition of a solution of (4-nitrophenyl)chloroformate (151.70 mg, 0.753 mmol, 3 eq.) in DCM (8 mL) at 0° C. After the completion of the addition, the mixture was warmed to 25° C. and stirred for 12 hours under N₂ atmosphere. The mixture was then poured into H₂O (50 mL) and extracted with EtOAc (50 mL×3). The combined organic layers were washed with brine (50 mL), dried over Na₂SO₄, filtered, and the filtrate was evaporated under reduced pressure.

The resulting residue was purified with Prep-TLC (PE:EA=1:1) to give tert-butyl (2-(4-((tert-butoxycarbonyl)amino)butoxy)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate (160 mg, 79.44% yield) as colorless gum. LCMS: 803.3 [M+H]⁺.

Step 8: Preparation of (1R,3S)-3-(5-((2-(4-aminobutoxy)-6-(difluoromethyl)pyridin-4-yl)amino)-1-(tert-butyl)-1H-pyrazol-3-yl)cyclopentyl (4-nitrophenyl) carbonate

The solution of tert-butyl (2-(4-((tert-butoxycarbonyl)amino)butoxy)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate (160 mg, 0.199 mmol, 1 eq.) and TFA (0.2 mL) in DCM (1 mL) was stirred at 25° C. for 1 hour under N₂ atmosphere. After the completion of the reaction, the mixture was evaporated under reduced pressure to give (1R,3S)-3-(5-((2-(4-aminobutoxy)-6-(difluoromethyl)pyridin-4-yl)amino)-1-(tert-butyl)-1H-pyrazol-3-yl)cyclopentyl (4-nitrophenyl) carbonate (100 mg, 83.27% yield) as a yellow gum. LCMS: 603.2 [M+H]⁺.

Step 9: Preparation of (11S,13R,Z)-21-(tert-butyl)-46-(difluoromethyl)-21H-5,12-dioxa-3,10-diaza-4(4,2)-pyridina-2(3,5)-pyrazola-1 (1,3)-cyclopentanacyclododecaphan-11-one

To a solution of (1R,3S)-3-(5-((2-(4-aminobutoxy)-6-(difluoromethyl)pyridin-4-yl)amino)-1-(tert-butyl)-1H-pyrazol-3-yl)cyclopentyl (4-nitrophenyl) carbonate (100 mg, 0.166 mmol, 1 eq.) in THF (150 mL) was slowly added DIEA (214.47 mg, 1.66 mmol, 289.04 μL, 10 eq.), and the mixture was stirred at 25° C. for 12 hours under N₂ atmosphere. The reaction mixture was then poured into H₂O (200 mL) and extracted with EtOAc (200 mL×3). The organic phase was washed with brine (50 mL), dried over Na₂SO₄, filtered, and the filtrate was evaporated under reduced pressure. The resulting residue was purified with Prep-TLC (PE:EA=1:1) to give (1¹S,1³R,Z)-2¹-(tert-butyl)-4⁶-(difluoromethyl)-2¹H-5,12-dioxa-3,10-diaza-4(4,2)-pyridina-2(3,5)-pyrazola-1(1,3)-cyclopentanacyclododecaphan-11-one (50 mg, 65.01% yield) as a yellow gum. LCMS: 464.4 [M+H]⁺.

Step 10: Preparation of (11S,13R,Z)-46-(difluoromethyl)-21H-5,12-dioxa-3,10-diaza-4(4,2)-pyridina-2(5,3)-pyrazola-1 (1,3)-cyclopentanacyclododecaphan-11-one

A solution of (1¹S,1³R,Z)-2¹-(tert-butyl)-4⁶-(difluoromethyl)-2¹H-5,12-dioxa-3,10-diaza-4(4,2)-pyridina-2(3,5)-pyrazola-1(1,3)-cyclopentanacyclododecaphan-11-one (70 mg, 0.151 mmol, 1 eq.) in HCOOH (5 mL) was stirred at 100° C. for 0.5 hour under N₂ atmosphere. The reaction solution was cooled to room temperature and concentrated under reduced pressure. The resulting residue was purified with Prep-HPCL (column: Waters Xbridge 150×25 mm, 5 mm; mobile phase: [water(FA)-ACN]; gradient: 23%-53% B over 9 min) to afford (1¹S,1³R,Z)-4⁶-(difluoromethyl)-2¹H-5,12-dioxa-3,10-diaza-4(4,2)-pyridina-2(5,3)-pyrazola-1(1,3)-cyclopentanacyclododecaphan-11-one (16.42 mg, 26.15% yield) as a white solid. LCMS: 408.1 [M+H]⁺; ¹H NMR (400 MHz, DMSO-d₆) δ 12.33-11.97 (m, 1H), 9.31-9.25 (m, 1H), 7.27-6.95 (m, 1H), 6.81-6.48 (m, 2H), 6.42 (s, 1H), 6.08 (s, 1H), 5.05 (br s, 1H), 4.23-3.87 (m, 2H), 3.21-2.97 (m, 2H), 2.91-2.78 (m, 1H), 2.20-1.91 (m, 2H), 1.80 (br d, J=14.1 Hz, 4H), 1.57 (br d, J=3.6 Hz, 2H), 1.41-1.07 (m, 2H) ppm.

Example 2: Synthesis of (1¹S,1³R,Z)-2¹H-12-oxa-3,10-diaza-4(4,2)-pyridina-2(3,5)-pyrazola-1(1,3)-cyclopentanacyclododecaphan-11-one (Compound 4)

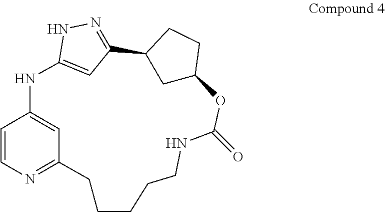 [SMILES: O=C1NCCCCCc2cc(ccn2)Nc2cc(n[nH]2)[C@H]2CC[C@H](C2)O1]

Step 1: Preparation of tert-butyl (5-(4-bromopyridin-2-yl)pent-4-yn-1-yl)carbamate

A mixture of 2,4-dibromopyridine (1.55 g, 6.55 mmol, 1 eq), tert-butyl pent-4-yn-1-ylcarbamate (1 g, 5.46 mmol, 0.83 eq), Pd(PPh₃)₄ (1.14 g, 0.982 mmol, 0.15 eq), CuI (374.15 mg, 1.96 mmol, 0.3 eq) and TEA (1.33 g, 13.10 mmol, 1.82 mL, 2 eq) in DMF (10 mL) was degassed and backfilled with N₂ for 3 times, and stirred at 40° C. for 12 hours under N₂ atmosphere. After the reaction was completed, the mixture was cooled to room temperature, filtered through a short pad of celite and rinsed with ethyl acetate (30 mL). The filtrate was washed with water (30 mL). The organic layer was collected, and the aqueous phase was extracted with ethyl acetate (30 mL×3). The combined organic layers were washed with brine (50 mL), dried over Na₂SO₄, filtered, and the filtrate was evaporated under reduced pressure. The resulting residue was purified by column chromatography to give tert-butyl (5-(4-bromopyridin-2-yl)pent-4-yn-1-yl)carbamate (1.1 g, 49.52% yield) as a yellow solid. LCMS: 339.0, 341.0[M+H]⁺; ¹H NMR (400 MHz, CDCl₃) δ 8.35 (br s, 1H), 7.57 (br s, 1H), 7.39 (br d, J=3.9 Hz, 1H), 4.71 (br s, 1H), 3.28 (q, J=6.0 Hz, 2H), 2.50 (t, J=7.0 Hz, 2H), 1.82 (quin, J=6.9 Hz, 2H), 1.44 (s, 9H) ppm.

Step 2: Preparation of tert-butyl (5-(4-((1-(tert-butyl)-3-((1S,3R)-3-((tert-butyldimethylsilyl)oxy)cyclopentyl)-1H-pyrazol-5-yl)amino)pyridin-2-yl)pent-4-yn-1-yl)carbamate

A mixture of tert-butyl (5-(4-bromopyridin-2-yl)pent-4-yn-1-yl)carbamate (300 mg, 0.884 mmol, 1 eq), 1-(tert-butyl)-3-((1S,3R)-3-((tert-butyldimethylsilyl)oxy)cyclopentyl)-1H-pyrazol-5-amine (298.54 mg, 0.884 mmol, 1 eq), Pd₂(dba)₃ (80.98 mg, 0.0884 mmol, 0.1 eq), Xantphos (84.32 mg, 0.177 mmol, 0.2 eq) and Cs₂CO₃ (864.43 mg, 2.65 mmol, 3 eq) in dioxane (4 mL) was heated and stirred at 90° C. for 12 hours under N₂ atmosphere. After the completion of the reaction, the mixture was cooled to room temperature, filtered through a short pad of celite and rinsed with dioxane (10 mL). The combined filtrate was concentrated under reduced pressure and the resulting residue was purified by column chromatography to give tert-butyl (5-(4-((1-(tert-butyl)-3-((1S,3R)-3-((tert-butyldimethylsilyl)oxy)cyclopentyl)-1H-pyrazol-5-yl)amino)pyridin-2-yl)pent-4-yn-1-yl)carbamate (284 mg, 45.27% yield) as a yellow oil. LCMS: 596.5 [M+H]⁺; 1H NMR (400 MHz, DMSO-d₆) δ 8.22 (s, 1H), 8.05 (d, J=5.6 Hz, 1H), 6.86 (br t, J=5.1 Hz, 1H), 6.54 (s, 1H), 6.48 (dd, J=2.1, 5.6 Hz, 1H), 5.99 (s, 1H), 4.35-4.28 (m, 1H), 3.04-2.95 (m, 3H), 2.40-2.34 (m, 2H), 2.26-2.19 (m, 1H), 1.95-1.89 (m, 1H), 1.81-1.74 (m, 2H), 1.66-1.58 (m, 4H), 1.47 (s, 9H), 1.37 (s, 9H), 0.83 (s, 9H), 0.03 (d, J=3.1 Hz, 6H) ppm.

Step 3: Preparation of tert-butyl (5-(4-((1-(tert-butyl)-3-((1S,3R)-3-((tert-butyldimethylsilyl)oxy)cyclopentyl)-1H-pyrazol-5-yl)amino)pyridin-2-yl)pentyl)carbamate

A mixture of tert-butyl (5-(4-((1-(tert-butyl)-3-((1S,3R)-3-((tert-butyldimethylsilyl)oxy)cyclopentyl)-1H-pyrazol-5-yl)amino)pyridin-2-yl)pent-4-yn-1-yl)carbamate (280 mg, 0.47 mmol, 1 eq), Pd/C (28.00 mg, 0.0263 mmol, 10% purity) and Pd(OH)₂/C (28.00 mg, 0.020 mmol, 10% purity) in MeOH (4 mL) was degassed and backfilled with H₂ for 3 times, and then the mixture was stirred at 60° C. for 3 hours under H₂ atmosphere. After the reaction was completed, the mixture was cooled to room temperature, filtered through a short pad of celite, rinsed with methanol (15 mL) and the filtrate was concentrated to give tert-butyl (5-(4-((1-(tert-butyl)-3-((1S,3R)-3-((tert-butyldimethylsilyl)oxy)cyclopentyl)-1H-pyrazol-5-yl)amino)pyridin-2-yl)pentyl)carbamate (250 mg, 88.69% yield) as a yellow oil, which was directly used in the next step. LCMS: 600.5 [M+H]⁺.

Step 4: Preparation of tert-butyl (2-(5-((tert-butoxycarbonyl)amino)pentyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (5-(4-((1-(tert-butyl)-3-((1S,3R)-3-((tert-butyldimethylsilyl)oxy)cyclopentyl)-1H-pyrazol-5-yl)amino)pyridin-2-yl)pentyl)carbamate (200 mg, 0.333 mmol, 1 eq) in THF (5 mL) was added Boc₂O (145.52 mg, 0.667 mmol, 153.18 μL, 2 eq), DMAP (40.73 mg, 0.333 mmol, 1 eq) and TEA (101.20 mg, 1.00 mmol, 139.21 μL, 3 eq). After the mixture was stirred at 25° C. for 5 hours under N₂ atmosphere, an additional portions of Boc₂O (145.52 mg, 0.667 mmol, 153.18 μL, 2 eq), DMAP (40.73 mg, 0.333 mmol, 1 eq) and TEA (101.20 mg, 1.00 mmol, 139.21 μL, 3 eq) was added and the mixture was kept stirring at 25° C. for additional 12 hours under N₂ atmosphere. The mixture was subsequently diluted with H₂O (30 mL) and extracted with ethyl acetate (15 mL×3). The combined organic layers were washed with brine (30 mL), dried over Na₂SO₄, filtered, and the filtrate was concentrated under reduced pressure. The resulting residue was purified by prep-TLC (SiO₂, PE:EA=1:1, R_{f}=0.81) to give tert-butyl (2-(5-((tert-butoxycarbonyl)amino)pentyl)pyridin-4-yl)(1-(tert-butyl)-3-((1S,3R)-3-((tert-butyldimethylsilyl)oxy)cyclopentyl)-1H-pyrazol-5-yl)carbamate (103 mg, 44.13% yield) as a colorless oil. LCMS: 586.4 [M+H]⁺.

Step 5: Preparation of tert-butyl (2-(5-((tert-butoxycarbonyl)amino)pentyl)pyridin-4-yl)(1-(tert-butyl)-3-((1S,3R)-3-hydroxycyclopentyl)-1H-pyrazol-5-yl)carbamate

To a solution of tert-butyl (2-(5-((tert-butoxycarbonyl)amino)pentyl)pyridin-4-yl)(1-(tert-butyl)-3-((1S,3R)-3-((tert-butyldimethylsilyl)oxy)cyclopentyl)-1H-pyrazol-5-yl)carbamate (103 mg, 0.147 mmol, 1 eq) in THF (3 mL) was added TBAF (1 M, 0.74 mmol, 735.68 μL, 5 eq), and the mixture was stirred at 25° C. for 16 hours under N₂ atmosphere. The mixture was then diluted with H₂O (30 mL) and extracted with ethyl acetate (10 mL×3). The combined organic layers were washed with brine (30 mL), dried over Na₂SO₄, filtered, and the filtrate was concentrated under reduced pressure. The resulting residue was purified by prep-TLC (SiO₂, PE:EA=1:1, R_{f}=0.17) to give tert-butyl (2-(5-((tert-butoxycarbonyl)amino)pentyl)pyridin-4-yl)(1-(tert-butyl)-3-((1S,3R)-3-hydroxycyclopentyl)-1H-pyrazol-5-yl)carbamate (92 mg, 95.00% yield) as a colorless oil. LCMS: 586.4 [M+H]⁺.

Step 6: Preparation of tert-butyl (2-(5-((tert-butoxycarbonyl)amino)pentyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(5-((tert-butoxycarbonyl)amino)pentyl)pyridin-4-yl)(1-(tert-butyl)-3-((1S,3R)-3-hydroxycyclopentyl)-1H-pyrazol-5-yl)carbamate (92 mg, 0.157 mmol, 1 eq) in THF (2 mL) was added pyridine (62.12 mg, 0.785 mmol, 63.38 μL, 5 eq) and DMAP (9.59 mg, 0.0785 mmol, 0.5 eq), followed by the addition of a solution of 4-nitrophenyl carbonochloridate (94.97 mg, 0.471 mmol, 3 eq) in DCM (2 mL) at 0° C. After the completion of the addition, the mixture was warmed to 25° C. and stirred for 16 hours under N₂ atmosphere. Then, an additional portion of (4-nitrophenyl)chloroformate (94.97 mg, 0.471 mmol, 3 eq) in mixed solvents of DCM (1 mL) and THF (1 mL) was added to the mixture. The mixture was kept stirring at 25° C. for an additional 16 hours under N₂ atmosphere. After the starting material was consumed, the mixture was diluted with H₂O (10 mL) and extracted with dichloromethane (10 mL×3). The combined organic layers were washed with brine (10 mL), dried over Na₂SO₄, filtered, and the filtrate was concentrated under reduced pressure. The resulting residue was purified by prep-TLC (SiO₂, PE:EA=1:1, R_{f}=0.89) to give tert-butyl (2-(5-((tert-butoxycarbonyl)amino)pentyl)pyridin-4-yl)(1-(tert-butyl)-3-((1S,3R)-3-(((4-nitrophenoxy)carbonyl)oxy)cyclopentyl)-1H-pyrazol-5-yl)carbamate (95 mg, 80.56% yield) as a yellow oil. LCMS: 751.5 [M+H]⁺.

Step 7: Preparation of (1R,3S)-3-(5-((2-(5-aminopentyl)pyridin-4-yl)amino)-1-(tert-butyl)-1H-pyrazol-3-yl)cyclopentyl (4-nitrophenyl) carbonate

The mixture of tert-butyl (2-(5-((tert-butoxycarbonyl)amino)pentyl)pyridin-4-yl)(1-(tert-butyl)-3-((1S,3R)-3-(((4-nitrophenoxy)carbonyl)oxy)cyclopentyl)-1H-pyrazol-5-yl)carbamate (75 mg, 0.10 mmol, 1 eq) and TFA in DCM (4 mL) was stirred at 30° C. for 12 hours under N₂ atmosphere. After the completion of the reaction, the mixture was evaporated to give (1R,3S)-3-(5-((2-(5-aminopentyl)pyridin-4-yl)amino)-1-(tert-butyl)-1H-pyrazol-3-yl)cyclopentyl (4-nitrophenyl) carbonate (55 mg, crude) as a yellow oil of crude TFA salt, which was directly used in the next step. LCMS: 551.3 [M+H]⁺.

Step 8: Preparation of (1¹S,1³R,Z)-2¹-(tert-butyl)-2¹H-12-oxa-3,10-diaza-4(4,2)-pyridina-2(3,5)-pyrazola-1 (1,3)-cyclopentanacyclododecaphan-11-one

To a solution of (1R,3S)-3-(5-((2-(5-aminopentyl)pyridin-4-yl)amino)-1-(tert-butyl)-1H-pyrazol-3-yl)cyclopentyl (4-nitrophenyl) carbonate (55 mg, 0.1 mmol, 1 eq) in THF (50 mL) was added DIPEA (64.54 mg, 0.50 mmol, 86.99 μL, 5 eq) at 0° C. The mixture was then warmed to 30° C. and stirred for 3 hours under N₂ atmosphere. After the completion of the reaction, the mixture was concentrated under reduced pressure. The resulting residue was purified by prep-TLC (SiO₂, PE:EA=0:1, R_{f}=0.22) to give 35 mg of the desired (1¹S,1³R,Z)-2¹-(tert-butyl)-2¹H-12-oxa-3,10-diaza-4(4,2)-pyridina-2(3,5)-pyrazola-1(1,3)-cyclopentanacyclododecaphan-11-one. LCMS: 412.4 [M+H]⁺.

Step 9: Preparation of (1¹S,1³R,Z)-2¹H-12-oxa-3,10-diaza-4(4,2)-pyridina-2(3,5)-pyrazola-1 (1,3)-cyclopentanacyclododecaphan-11-one

A solution of (1¹S,1³R,Z)-2¹-(tert-butyl)-2¹H-12-oxa-3,10-diaza-4(4,2)-pyridina-2(3,5)-pyrazola-1(1,3)-cyclopentanacyclododecaphan-11-one (80 mg, 0.194 mmol, 1 eq) in HCOOH (4 mL) was stirred at 100° C. for 0.5 hour under N₂ atmosphere. After the completion of the reaction, the mixture was cooled to room temperature and the volatiles were evaporated under reduced pressure. The resulting residue was purified by prep-HPLC (column: Phenomenex Luna C18 150×25 mm, 10 um; mobile phase: [water (FA)-ACN]; gradient: 5%-35% B over 10 min) to afford (1¹S,1³R,Z)-2¹H-12-oxa-3,10-diaza-4(4,2)-pyridina-2(3,5)-pyrazola-1(1,3)-cyclopentanacyclododecaphan-11-one (23.14 mg, 32.82% yield) as a white solid. LCMS: 356.3 [M+H]⁺; ¹H NMR (400 MHz, DMSO-d₆) δ 8.91-8.79 (m, 1H), 8.18 (s, 1H), 8.01 (d, J=5.75 Hz, 1H), 7.35-6.92 (m, 1H), 6.72-6.81 (m, 1H), 6.59 (dd, J=5.63, 2.13 Hz, 1H), 5.94 (s, 1H), 5.14-4.90 (m, 1H), 2.97-2.90 (m, 1H), 2.79-2.72 (m, 1H), 2.63-2.56 (m, 2H), 2.05-1.99 (m, 1H), 1.86-1.57 (m, 7H), 1.48-1.22 (m, 5H) ppm.

Example 3: Synthesis of (1¹S,1³R,Z)-9-methyl-2¹H-5,12-dioxa-3,10-diaza-4(4,2)-pyridina-2(3,5)-pyrazola-1(1,3)-cyclopentanacyclododecaphan-11-one (Compound 8), (1¹S,1³R,9S,Z)-9-methyl-2¹H-5,12-dioxa-3,10-diaza-4(4,2)-pyridina-2(3,5)-pyrazola-1(1,3)-cyclopentanacyclododecaphan-11-one (Compound 9) and (1¹S,1³R,9R,Z)-9-methyl-2¹H-5,12-dioxa-3,10-diaza-4(4,2)-pyridina-2(3,5)-pyrazola-1(1,3)-cyclopentanacyclododecaphan-11-one (Compound 10)

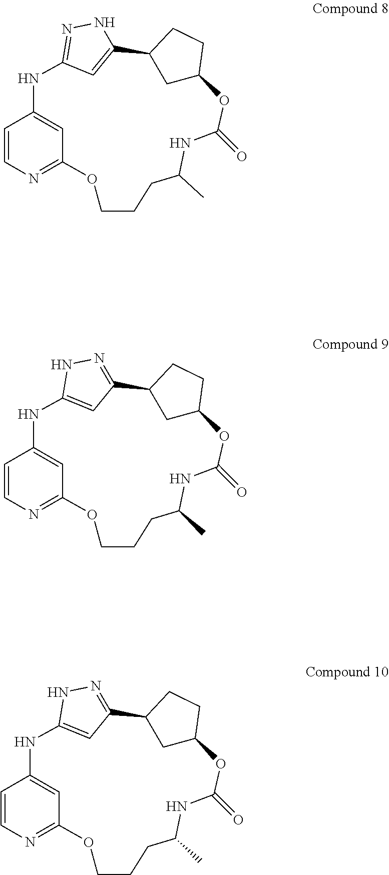 [SMILES: CC1CCCOc2cc(ccn2)Nc2cc([nH]n2)[C@H]2CC[C@H](C2)OC(=O)N1 ; C[C@H]1CCCOc2cc(ccn2)Nc2cc(n[nH]2)[C@H]2CC[C@H](C2)OC(=O)N1 ; C[C@@H]1CCCOc2cc(ccn2)Nc2cc(n[nH]2)[C@H]2CC[C@H](C2)OC(=O)N1]

### Synthetic Route 1:

Step 1: Preparation of 4-aminopentan-1-ol

A mixture of 5-hydroxypentan-2-one (5 g, 48.96 mmol, 4.97 mL, 1 eq), NH₃·H₂O (46.37 g, 489.57 mmol, 50.96 mL, 37% purity, 10 eq) and Raney-Ni (0.5 g, 5.84 mmol) in 100 mL of 50% aqueous ethanol was degassed and backfilled with H₂ for 3 times, and the mixture was stirred at 80° C. for 12 hours under H₂ (50 Psi) atmosphere. After the completion of the reaction, the reaction mixture was cooled to room temperature, filtered through a short pad of celite, rinsed with ethanol, and the filtrate was concentrated to give 4-aminopentan-1-ol (4.7 g, 93.06% yield) as a white solid. ¹H NMR (400 MHz, CHLOROFORM-d) δ 3.93-3.48 (m, 2H), 3.21-3.10 (m, 2H), 3.08-2.80 (m, 1H), 1.82-1.58 (m, 2H), 1.29-0.77 (m, 3H) ppm.

Step 2: Preparation of tert-butyl (5-hydroxypentan-2-yl)carbamate

To a solution of 4-aminopentan-1-ol (500 mg, 4.85 mmol, 1 eq) in DCM (6 mL) was added TEA (1.47 g, 14.54 mmol, 2.02 mL, 3 eq) and Boc₂O (3.17 g, 14.54 mmol, 3 eq) at 0° C., and the mixture was warmed to 25° C. and kept stirring for 12 hours under N₂ atmosphere. After the completion of the reaction, the mixture was concentrated under reduced pressure. The resulting residue was treated with 10 mL of water and extracted with ethyl acetate (10 mL×3). The combined organic layers were dried over Na₂SO₄, filtered, and the filtrate was concentrated. The resulting residue was purified by prep-TLC (SiO₂, PE:EA=3:1, R_{f}=0.6) to give tert-butyl (5-hydroxypentan-2-yl)carbamate (450 mg, 45.67% yield) as a colorless oil. ¹H NMR (400 MHz, CHLOROFORM-d) δ 4.38-4.26 (m, 1H), 4.07 (t, J=6.5 Hz, 2H), 3.68 (br t, J=5.8 Hz, 2H), 1.78-1.67 (m, 2H), 1.61 (br d, J=5.1 Hz, 2H), 1.49 (s, 9H), 1.15-1.13 (m, 3H) ppm.

Step 3: Preparation of tert-butyl (5-((4-bromopyridin-2-yl)oxy)pentan-2-yl)carbamate

To a solution of tert-butyl (5-hydroxypentan-2-yl)carbamate (350 mg, 1.72 mmol, 1 eq) in THF (6 mL) was added 4-bromo-2-fluoro-pyridine (333.31 mg, 1.89 mmol, 1.1 eq) and t-BuOK (289.81 mg, 2.58 mmol, 1.5 eq) at 0° C., and the mixture was stirred at 25° C. for 12 hours under N₂ atmosphere. After the starting material was consumed, the volatiles were evaporated under reduced pressure. The resulting residue was diluted with water (30 mL) and extracted with ethyl acetate (30 mL×3). The combined organic layers were washed with brine (20 mL), dried over Na₂SO₄, filtered, and the filtrate was concentrated under reduced pressure. The resulting residue was purified by column chromatography to give tert-butyl (5-((4-bromopyridin-2-yl)oxy)pentan-2-yl)carbamate (200 mg, 32.33% yield) as a colorless oil. ¹H NMR (400 MHz, CHLOROFORM-d) δ 8.05-7.90 (m, 1H), 7.02 (dd, J=1.5, 5.5 Hz, 1H), 6.95 (s, 1H), 4.45-4.33 (m, 1H), 4.30 (t, J=6.3 Hz, 2H), 4.12-4.03 (m, 1H), 3.75-3.63 (m, 1H), 1.87-1.77 (m, 2H), 1.74-1.67 (m, 1H), 1.45 (s, 9H), 1.17-1.14 (m, 3H) ppm.

Step 4: Preparation of tert-butyl (5-((4-((1-(tert-butyl)-3-((1S,3R)-3-((tert-butyldimethylsilyl)oxy)cyclopentyl)-1H-pyrazol-5-yl)amino)pyridin-2-yl)oxy)pentan-2-yl)carbamate

The mixture of tert-butyl (5-((4-bromopyridin-2-yl)oxy)pentan-2-yl)carbamate (200 mg, 0.557 mmol, 1 eq), 1-(tert-butyl)-3-((1S,3R)-3-((tert-butyldimethylsilyl)oxy)cyclopentyl)-1H-pyrazol-5-amine (187.93 mg, 0.557 mol, 1 eq), Pd₂(dba)₃ (50.98 mg, 0.0557 mmol, 0.1 eq), Xantphos (53.08 mg, 0.111 mmol, 0.2 eq) and Cs₂CO₃ (544.15 mg, 1.67 mmol, 3 eq) in dioxane (5 mL) was heated and stirred at 90° C. for 12 hours under N₂ atmosphere. After the completion of the reaction, the mixture was cooled to room temperature, filtered through a short pad of Celite and rinsed with dioxane (10 mL), and the filtrate was concentrated. The resulting crude was purified by column chromatography to give tert-butyl (5-((4-((1-(tert-butyl)-3-((1S,3R)-3-((tert-butyldimethylsilyl)oxy)cyclopentyl)-1H-pyrazol-5-yl)amino)pyridin-2-yl)oxy)pentan-2-yl)carbamate (250 mg, 72.91% yield) as a yellow oil. LCMS: 616.6 [M+H]⁺; ¹H NMR (400 MHz, CHLOROFORM-d) δ 7.92-7.76 (m, 1H), 6.39-6.19 (m, 1H), 6.12-5.94 (m, 2H), 4.48-4.36 (m, 1H), 4.35-4.30 (m, 1H), 4.29-4.21 (m, 2H), 3.80-3.60 (m, 1H), 3.16-2.97 (m, 1H), 2.40-2.25 (m, 1H), 2.03-1.95 (m, 1H), 1.90-1.86 (m, 1H), 1.85-1.76 (m, 4H), 1.75-1.64 (m, 4H), 1.58 (s, 9H), 1.44 (s, 9H), 1.15 (d, J=6.6 Hz, 3H), 0.88 (s, 9H), 0.06 (d, J=2.8 Hz, 6H) ppm.

Step 5: Preparation of tert-butyl (2-((4-((tert-butoxycarbonyl)amino)pentyl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (5-((4-((1-(tert-butyl)-3-((1S,3R)-3-((tert-butyldimethylsilyl)oxy)cyclopentyl)-1H-pyrazol-5-yl)amino)pyridin-2-yl)oxy)pentan-2-yl)carbamate (250 mg, 0.406 mmol, 1 eq) in THF (5 mL) was added Boc₂O (177.17 mg, 0.812 mmol, 186.50 μL, 2 eq), TEA (123.22 mg, 1.22 mmol, 169.49 μL, 3 eq) and DMAP (49.59 mg, 0.406 mmol, 1 eq) at 0° C., and the mixture was slowly warmed to 25° C. and kept stirring for 12 hours under N₂ atmosphere. After the completion of the reaction, the mixture was concentrated under reduced pressure. The resulting residue was purified by column chromatography to give tert-butyl (2-((4-((tert-butoxycarbonyl)amino)pentyl)oxy)pyridin-4-yl)(1-(tert-butyl)-3-((1S,3R)-3-((tert-butyldimethylsilyl)oxy)cyclopentyl)-1H-pyrazol-5-yl)carbamate (230 mg, 79.14% yield) as a yellow oil. LCMS: 716.6 [M+H]⁺; ¹H NMR (400 MHz, CDCl₃) δ 8.00 (br d, J=6.0 Hz, 1H), 6.91-6.83 (m, 1H), 6.83-6.77 (m, 1H), 6.03-5.97 (m, 1H), 4.40-4.33 (m, 1H), 4.28 (br s, 2H), 3.77-3.62 (m, 1H), 3.16-3.02 (m, 1H), 2.42-2.29 (m, 1H), 1.89-1.77 (m, 4H), 1.71-1.55 (m, 6H), 1.46 (s, 9H), 1.46-1.43 (m, 18H), 1.17-1.11 (m, 3H), 0.90-0.88 (m, 9H), 0.07 (d, J=2.3 Hz, 6H) ppm.

Step 6: Preparation of tert-butyl (2-((4-((tert-butoxycarbonyl)amino)pentyl)oxy)pyridin-4-yl)(1-(tert-butyl)-3-((1S,3R)-3-hydroxycyclopentyl)-1H-pyrazol-5-yl)carbamate

The solution of tert-butyl (2-((4-((tert-butoxycarbonyl)amino)pentyl)oxy)pyridin-4-yl)(1-(tert-butyl)-3-((1S,3R)-3-((tert-butyldimethylsilyl)oxy)cyclopentyl)-1H-pyrazol-5-yl)carbamate (230 mg, 0.321 mmol, 1 eq) and TBAF (1 M, 1.61 mL, 5 eq) in THF (8 mL) was stirred at 25° C. for 12 hours under N₂ atmosphere, and the mixture was then concentrated under reduced pressure. The resulting residue was purified by column chromatography to give tert-butyl (2-((4-((tert-butoxycarbonyl)amino)pentyl)oxy)pyridin-4-yl)(1-(tert-butyl)-3-((1S,3R)-3-hydroxycyclopentyl)-1H-pyrazol-5-yl)carbamate (135 mg, 69.84% yield) as a yellow oil. LCMS: 602.5 [M+H]⁺; ¹H NMR (400 MHz, CHLOROFORM-d) δ 8.01 (d, J=5.9 Hz, 1H), 6.87-6.82 (m, 1H), 6.80-6.76 (m, 1H), 5.96 (d, J=2.3 Hz, 1H), 5.31 (s, 1H), 4.39 (br s, 2H), 4.28 (t, J=6.4 Hz, 2H), 3.77-3.63 (m, 1H), 3.38-3.21 (m, 1H), 2.22-2.08 (m, 2H), 2.00-1.88 (m, 2H), 1.82-1.77 (m, 2H), 1.63-1.55 (m, 4H), 1.46 (s, 9H), 1.45 (br s, 9H), 1.44 (s, 9H), 1.15 (d, J=6.6 Hz, 3H) ppm.

Step 7: Preparation of tert-butyl (2-((4-((tert-butoxycarbonyl)amino)pentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((4-((tert-butoxycarbonyl)amino)pentyl)oxy)pyridin-4-yl)(1-(tert-butyl)-3-((1S,3R)-3-hydroxycyclopentyl)-1H-pyrazol-5-yl)carbamate (135 mg, 0.224 mmol, 1 eq) in THF (3 mL) was added Py (88.72 mg, 1.12 mmol, 90.54 μL, 5 eq) and DMAP (13.70 mg, 0.112 mmol, 0.5 eq), followed by the addition of a solution of 4-nitrophenyl carbonochloridate (135.65 mg, 0.673 mmol, 3 eq) in DCM (3 mL) at 0° C. After the mixture was stirred at 25° C. for 5 hours under N₂ atmosphere, an additional solution of 4-nitrophenyl carbonochloridate (135.65 mg, 0.673 mmol, 3 eq) in DCM (3 mL) was added. The mixture was kept stirring at 25° C. for an additional 12 hours under N₂ atmosphere and the volatiles were evaporated. The resulting residue was diluted with water (10 mL) and extracted with ethyl acetate (10 mL×3). The combined organic layers were washed with brine (10 mL), dried over Na₂SO₄, filtered, and the filtrate was concentrated under reduced pressure. The resulting residue was purified by column chromatography to tert-butyl (2-((4-((tert-butoxycarbonyl)amino)pentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (100 mg) as a white solid. LCMS: 767.5 [M+H]⁺.

Step 8: Preparation of (1R,3S)-3-(5-((2-((4-aminopentyl)oxy)pyridin-4-yl)amino)-1-(tert-butyl)-1H-pyrazol-3-yl)cyclopentyl (4-nitrophenyl) carbonate

The mixture of tert-butyl (2-((4-((tert-butoxycarbonyl)amino)pentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (80 mg, 0.104 mmol, 1 eq) and TFA (614.00 mg, 5.38 mmol, 400.00 μL, 51.62 eq) in THF (2 mL) was stirred at 30° C. for 12 hours under N₂ atmosphere. After the completion of the reaction, the volatiles were removed under reduced pressure. The resulting residue was purified by prep-HPLC (column: Phenomenex Luna C18 150×25 mm, 10 mm; mobile phase: [water (TFA)-ACN]; gradient:18%-48% B over 9 min) to give (1R,3S)-3-(5-((2-((4-aminopentyl)oxy)pyridin-4-yl)amino)-1-(tert-butyl)-1H-pyrazol-3-yl)cyclopentyl (4-nitrophenyl) carbonate (35 mg, 59.21% yield) as a colorless oil. LCMS: 567.4 [M+H]⁺.

Step 9: Preparation of (1¹S,1³R,Z)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-11-one

To a solution of (1R,3S)-3-(5-((2-((4-aminopentyl)oxy)pyridin-4-yl)amino)-1-(tert-butyl)-1H-pyrazol-3-yl)cyclopentyl (4-nitrophenyl) carbonate (35 mg, 0.0618 mmol, 1 eq) in THF (30 mL) was added DIPEA (39.91 mg, 0.309 mmol, 53.79 μL, 5 eq), and the mixture was stirred at 30° C. for 24 hours under N₂ atmosphere. The mixture was then concentrated under reduced pressure. The resulting residue was purified by prep-TLC (SiO₂, DCM:MeOH=10:1, R_{f}=0.4) to give (1¹S,1³R,Z)-2¹-(tert-butyl)-9-methyl-2¹H-5,12-dioxa-3,10-diaza-4(4,2)-pyridina-2(3,5)-pyrazola-1 (1,3)-cyclopentanacyclododecaphan-11-one (26 mg, 98.46% yield) as a colorless oil. LCMS: 428.3 [M+H]⁺.

Step 10: Preparation of (1¹S,1³R,Z)-9-methyl-2¹H-5,12-dioxa-3,10-diaza-4(4,2)-pyridina-2(3,5)-pyrazola-1 (1,3)-cyclopentanacyclododecaphan-11-one (Compound 8)

A mixture of (1¹S,1³R,Z)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-11-one (26 mg, 0.0608 mmol, 1 eq) in formic acid (3 mL) was stirred at 100° C. for 0.5 hour under N₂ atmosphere. After the completion of the reaction, the reaction mixture was cooled to room temperature, and the volatiles were removed under reduced pressure. The resulting residue was purified by prep-HPLC (column: Phenomenex Luna C18 150×25 mm, 10 mm; mobile phase: [water (FA)-ACN]; gradient:8%-38% B over 7 min) to give (1¹S,1³R,Z)-9-methyl-2¹H-5,12-dioxa-3,10-diaza-4(4,2)-pyridina-2(3,5)-pyrazola-1(1,3)-cyclopentanacyclododecaphan-11-one (Compound 8) (12 mg, 45.09% yield) as a white solid. LCMS: 372.2 [M+H]⁺; ¹H NMR (400 MHz, DMSO-d₆) δ 12.02 (s, 1H), 8.64 (s, 1H), 8.17 (s, 1H), 7.74 (d, J=5.8 Hz, 1H), 6.47-6.37 (m, 2H), 6.34 (s, 1H), 6.11-5.98 (m, 1H), 5.19-4.99 (m, 1H), 4.21-3.97 (m, 2H), 3.71-3.49 (m, 1H), 3.28-3.20 (m, 1H), 2.44-2.33 (m, 1H), 2.07-1.95 (m, 1H), 1.88-1.66 (m, 6H), 1.65-1.45 (m, 2H), 1.12-0.96 (m, 3H) ppm.

Step 11: Preparation of (1¹S,1³R,9S,Z)-9-methyl-2¹H-5,12-dioxa-3,10-diaza-4(4,2)-pyridina-2(3,5)-pyrazola-1(1,3)-cyclopentanacyclododecaphan-11-one and (1¹S,1³R,9R,Z)-9-methyl-2¹H-5,12-dioxa-3,10-diaza-4(4,2)-pyridina-2(3,5)-pyrazola-1(1,3)-cyclopentanacyclododecaphan-11-one

The racemic mixture of (1¹S,1³R,Z)-9-methyl-2¹H-5,12-dioxa-3,10-diaza-4(4,2)-pyridina-2(3,5)-pyrazola-1(1,3)-cyclopentanacyclododecaphan-11-one (8 mg, 6.04 μmol, 90.024% purity) was further separated by chiral SFC (condition: column: DAICEL CHIRALPAK IC (250 mm×30 mm, 10 mm); mobile phase: [CO₂-ACN/MeOH (0.1% NH₃/H₂O)]; B %:45%, isocratic elution mode) to give (1¹S,1³R,9S,Z)-9-methyl-2¹H-5,12-dioxa-3,10-diaza-4(4,2)-pyridina-2(3,5)-pyrazola-1 (1,3)-cyclopentanacyclododecaphan-11-one (2.49 mg, 44.02% yield) as a white solid and (1¹S,1³R,9R,Z)-9-methyl-2¹H-5,12-dioxa-3,10-diaza-4(4,2)-pyridina-2(3,5)-pyrazola-1(1,3)-cyclopentanacyclododecaphan-11-one (2.52 mg, 44.30% yield) as a white solid.

(1¹S,1³R,9S,Z)-9-methyl-2¹H-5,12-dioxa-3,10-diaza-4(4,2)-pyridina-2(3,5)-pyrazola-1(1,3)-cyclopentanacyclododecaphan-11-one: LCMS: 372.2 [M+H]⁺; ¹H NMR (400 MHz, DMSO-d₆) δ 12.09 (s, 1H), 8.91 (s, 1H), 7.73 (d, J=5.6 Hz, 1H), 6.95 (br d, J=8.5 Hz, 1H), 6.42 (d, J=5.6 Hz, 1H), 6.32 (s, 1H), 5.99 (s, 1H), 5.01 (br s, 1H), 4.03 (br t, J=6.6 Hz, 2H), 3.71-3.56 (m, 1H), 3.23-3.15 (m, 1H), 2.41-2.34 (m, 1H), 2.03-1.98 (m, 1H), 1.85-1.68 (m, 6H), 1.53-1.44 (m, 2H), 1.23 (br s, 3H) ppm.

(1¹S,1³R,9R,Z)-9-methyl-2¹H-5,12-dioxa-3,10-diaza-4(4,2)-pyridina-2(3,5)-pyrazola-1(1,3)-cyclopentanacyclododecaphan-11-one: LCMS: 372.2 [M+H]⁺; ¹H NMR (400 MHz, DMSO-d₆) δ 12.09 (s, 1H), 8.92 (br s, 1H), 7.73 (br d, J=5.3 Hz, 1H), 6.86-6.58 (m, 1H), 6.42 (br d, J=5.1 Hz, 1H), 6.37 (s, 1H), 6.20-5.96 (m, 1H), 5.23-4.89 (m, 1H), 4.16-3.94 (m, 2H), 3.50 (br d, J=7.9 Hz, 1H), 3.20-3.14 (m, 1H), 2.21-2.09 (m, 1H), 2.03-1.96 (m, 1H), 1.90-1.65 (m, 6H), 1.63 (br d, J=6.3 Hz, 2H), 1.23 (br s, 3H) ppm.

### Chiral Synthetic Route 2:

Step 1: Preparation of tert-butyl (S)-2-methyl-5-oxopyrrolidine-1-carboxylate

To a solution of (S)-5-methylpyrrolidin-2-one (20 g, 201.75 mmol, 1 eq) in acetonitrile (400 mL) was added Boc₂O (52.84 g, 242.10 mmol, 1.2 eq) and DMAP (4.93 g, 40.35 mmol, 0.2 eq) at room temperature, and the mixture was stirred for 4 hours at 25° C. After the completion of the reaction, the reaction mixture was concentrated under reduced pressure. The resulting residue was purified by column chromatography to give tert-butyl (S)-2-methyl-5-oxopyrrolidine-1-carboxylate (40 g, 99.5% yield) as a colorless oil. LCMS: 421.3 [M+H]⁺.

Step 2: Preparation of methyl (S)-4-((tert-butoxycarbonyl)amino)pentanoate

To a solution of tert-butyl (S)-2-methyl-5-oxopyrrolidine-1-carboxylate (40 g, 200.76 mmol, 1 eq) in a mixed solvent of DCM (600 mL) and MeOH (120 mL) was added potassium carbonate (33.29 g, 240.91 mmol, 1.2 eq), and the mixture was stirred at 25° C. for 12 hours. After the completion of the reaction, the mixture was concentrated under reduced pressure. The resulting residue was diluted with 800 mL of water and extracted with DCM (500 mL×2). The combined organic layers were dried over Na₂SO₄, filtered and the filtrate was concentrated under reduced pressure to give 46 g of methyl (S)-4-((tert-butoxycarbonyl)amino)pentanoate as a yellow solid, which was used in the next step without further purification. ¹H NMR (400 MHz, CHLOROFORM-d) δ 4.43-4.31 (m, 1H), 3.67 (s, 3H), 2.37 (t, J=7.6 Hz, 2H), 1.84-1.74 (m, 1H), 1.71-1.64 (m, 1H), 1.43 (s, 9H), 1.14 (d, J=6.6 Hz, 3H) ppm.

Step 3: Preparation of tert-butyl (S)-(5-hydroxypentan-2-yl)carbamate

To a solution of methyl (S)-4-((tert-butoxycarbonyl)amino)pentanoate (20 g, 86.47 mmol, 1 eq) in 200 mL of THF was added dropwise LiBH₄ (4 M in THF, 64.85 mL, 3 eq) at 0° C. under N₂ atmosphere. After the completion of addition, the resulting mixture was stirred at 50° C. for 3 hours under N₂ atmosphere. The mixture was then cooled to room temperature and carefully quenched with saturated aqueous NH₄Cl (1 M, 300 mL). The mixture was extracted with EtOAc (300 mL×2). The combined organic layers were dried over Na₂SO₄, filtered and the filtrate was concentrated under reduced pressure to give tert-butyl (S)-(5-hydroxypentan-2-yl)carbamate (17.5 g, 99.6% yield) as a yellow oil, which was used in the next step without further purification. 1H NMR (400 MHz, CHLOROFORM-d) δ 4.48-4.32 (m, 1H), 3.73-3.62 (m, 3H), 1.89-1.80 (m, 2H), 1.66-1.56 (m, 2H), 1.44 (s, 8H), 1.13 (d, J=6.6 Hz, 3H) ppm.

Step 4: Preparation of tert-butyl (S)-(5-((4-bromopyridin-2-yl)oxy)pentan-2-yl)carbamate

To a solution of tert-butyl (S)-(5-hydroxypentan-2-yl)carbamate (17.5 g, 86.09 mmol, 1 eq) and 4-bromo-2-fluoro-pyridine (15.15 g, 86.09 mmol, 1 eq) in THF (350 mL) was added t-BuOK (9.66 g, 86.09 mmol, 1 eq) in portions at 0° C. under N₂ atmosphere. After the completion of the addition, the mixture was warmed to 25° C. and stirred for 2 hours under N₂ atmosphere. The mixture was then quenched with saturated aqueous NH₄Cl (400 mL) and extracted with EtOAc (500 mL×2). The combined organic layers were washed with brine (100 mL), dried over anhydrous Na₂SO₄, filtered and the filtrate was concentrated in vacuo. The resulting residue was purified by column chromatography to afford tert-butyl (S)-(5-((4-bromopyridin-2-yl)oxy)pentan-2-yl)carbamate (26 g, 84.1% yield) as a yellow oil. LCMS: 359.0, 361.0 [M+H]⁺.

Step 5: Preparation of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)pentan-2-yl)carbamate

A mixture of tert-butyl (S)-(5-((4-bromopyridin-2-yl)oxy)pentan-2-yl)carbamate (10.00 g, 27.84 mmol, 1 eq), 1-(tert-butyl)-3-((1S,3R)-3-((tert-butyldimethylsilyl)oxy)cyclopentyl)-1H-pyrazol-5-amine (10.00 g, 29.62 mmol, 1.06 eq), Xantphos (3.22 g, 5.57 mmol, 0.2 eq), Pd₂(dba)₃ (2.55 g, 2.78 mmol, 0.1 eq) and Cs₂CO₃ (18.14 g, 55.67 mmol, 2 eq) in dioxane (200 mL) was degassed and back-filled with N₂ for three times, and stirred at 90° C. for 4 hours under N₂ atmosphere. After the completion of the reaction, the mixture was cooled to room temperature. The mixture was filtered through a pad of the Celite, rinsed with ethyl acetate (100 mL). The combined filtrate was concentrated in vacuo. The resulting residue was purified by column chromatography to afford tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)pentan-2-yl)carbamate (14.37 g, 83.6% yield) as a yellow oil. LCMS: 616.6 [M+H]⁺.

Step 6: Preparation of tert-butyl (2-(((S)-4-((tert-butoxycarbonyl)amino)pentyl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)pentan-2-yl)carbamate (14.37 g, 23.33 mmol, 1 eq) in DCM (280 mL) were added DMAP (1.43 g, 11.67 mmol, 0.5 eq) and Boc₂O (7.64 g, 35.00 mmol, 1.5 eq), and the reaction mixture was stirred at 25° C. for 12 hours under N₂ atmosphere. After the completion of the reaction, the mixture was concentrated in vacuo. The resulting residue was purified by column chromatography to afford tert-butyl (2-(((S)-4-((tert-butoxycarbonyl)amino)pentyl)oxy)pyridin-4-yl)(1-(tert-butyl)-3-((1S,3R)-3-((tert-butyldimethylsilyl)oxy)cyclopentyl)-1H-pyrazol-5-yl)carbamate (15.14 g, 90.6% yield) as a yellow oil. LCMS: 716.4 [M+H]⁺.

Step 7: Preparation of tert-butyl (2-(((S)-4-((tert-butoxycarbonyl)amino)pentyl)oxy)pyridin-4-yl)(1-(tert-butyl)-3-((1S,3R)-3-hydroxycyclopentyl)-1H-pyrazol-5-yl)carbamate

To a solution of tert-butyl (2-(((S)-4-((tert-butoxycarbonyl)amino)pentyl)oxy)pyridin-4-yl)(1-(tert-butyl)-3-((1S,3R)-3-((tert-butyldimethylsilyl)oxy)cyclopentyl)-1H-pyrazol-5-yl)carbamate (15.14 g, 21.14 mmol, 1 eq) in THF (300 mL) was added TBAF (1 M, 63.43 mL, 3 eq), and the reaction mixture was stirred at 25° C. for 12 hours under N₂ atmosphere. The mixture was then diluted with H₂O (300 mL) and extracted with EtOAc (400 mL×2). The combined organic layers were washed with brine (50 mL×2), dried over anhydrous Na₂SO₄, filtered and the filtrate was concentrated in vacuo to give 11.7 g of crude tert-butyl (2-(((S)-4-((tert-butoxycarbonyl)amino)pentyl)oxy)pyridin-4-yl)(1-(tert-butyl)-3-((1S,3R)-3-hydroxycyclopentyl)-1H-pyrazol-5-yl)carbamate as a yellow oil, which was used in the next step without further purification. LCMS: 602.5 [M+H]⁺.

Step 8: Preparation of tert-butyl (2-(((S)-4-((tert-butoxycarbonyl)amino)pentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(((S)-4-((tert-butoxycarbonyl)amino)pentyl)oxy)pyridin-4-yl)(1-(tert-butyl)-3-((1S,3R)-3-hydroxycyclopentyl)-1H-pyrazol-5-yl)carbamate (11.7 g, 19.44 mmol, 1 eq), (4-nitrophenyl) carbonochloridate (11.76 g, 58.33 mmol, 3 eq) and DMAP (2.38 g, 19.44 mmol, 1 eq) in DCM (300 mL) was added pyridine (9.23 g, 116.65 mmol, 6 eq) dropwise at 25° C. After the completion of the addition, the reaction mixture was kept stirring for 0.5 hour at that temperature under N₂ atmosphere. The mixture was filtered, and the filtrate was concentrated in vacuo. The resulting residue was purified by column chromatography to afford tert-butyl (2-(((S)-4-((tert-butoxycarbonyl)amino)pentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (14 g, 93.9% yield) as a light yellow oil. LCMS: 767.5 [M+H]⁺.

Step 9: Preparation of (1R,3S)-3-(5-((2-(((S)-4-aminopentyl)oxy)pyridin-4-yl)amino)-1-(tert-butyl)-1H-pyrazol-3-yl)cyclopentyl (4-nitrophenyl) carbonate

To a solution of tert-butyl (2-(((S)-4-((tert-butoxycarbonyl)amino)pentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (14.00 g, 18.26 mmol, 1 eq) in DCM (200 mL) was added TFA (85.81 g, 752.60 mmol, 41.23 eq) and the mixture was stirred at 25° C. for 12 hours under N₂ atmosphere. After the completion of the reaction, the mixture was concentrated in vacuo to afford 10.3 g of crude (1R,3S)-3-(5-((2-(((S)-4-aminopentyl)oxy)pyridin-4-yl)amino)-1-(tert-butyl)-1H-pyrazol-3-yl)cyclopentyl (4-nitrophenyl) carbonate as a yellow oil, which was used in the next step without further purification. LCMS: 567.3 [M+H]⁺.

Step 10: Preparation of (1¹S,1³R,9S,Z)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-11-one

To a solution of (1R,3S)-3-(5-((2-(((S)-4-aminopentyl)oxy)pyridin-4-yl)amino)-1-(tert-butyl)-1H-pyrazol-3-yl)cyclopentyl (4-nitrophenyl) carbonate (10.3 g, 17.65 mmol, 1 eq) in THF (5 L) was added DIEA (11.4 g, 88.23 mmol, 5 eq), and the mixture was stirred at 50° C. for 12 hours under N₂ atmosphere. After the completion of the reaction, the mixture was cooled to room temperature and concentrated under reduced pressure. The resulting residue was diluted with H₂O (200 mL) and extracted with EtOAc (300 mL×2). The combined organic layers were washed with brine (100 mL), dried over anhydrous Na₂SO₄, filtered and the filtrate was concentrated in vacuo. The resulting residue was purified by column chromatography to afford (1¹S,1³R,9S,Z)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-11-one (6.4 g, 84.8% yield) as a white solid. LCMS: 428.4 [M+H]⁺.

Step 11: Preparation of (1¹S,1³R,9S,Z)-9-methyl-2¹H-5,12-dioxa-3,10-diaza-4(4,2)-pyridina-2(3,5)-pyrazola-1 (1,3)-cyclopentanacyclododecaphan-11-one (Compound 9)

A mixture of (1¹S,1³R,9S,Z)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-11-one (5.0 g, 11.69 mmol, 1 eq) and formic acid (100 mL) was stirred at 100° C. for 3 hours under N₂ atmosphere. After the completion of the reaction, the mixture was cooled to room temperature and concentrated in vacuo. The resulting residue was purified by prep-HPLC (column: Welch Ultimate XB—SiOH 250×70×10 um; mobile phase: [Hexane-EtOH]; gradient:5%-45% B over 15 min) to afford (1¹S,1³R,9S,Z)-9-methyl-2¹H-5,12-dioxa-3,10-diaza-4(4,2)-pyridina-2(3,5)-pyrazola-1(1,3)-cyclopentanacyclododecaphan-11-one (4.27 g, 94.2% yield) as an off-white solid. LCMS: 372.2 [M+H]⁺; ¹H NMR (400 MHz, DMSO-d₆) δ 12.29-11.82 (m, 1H), 8.91 (s, 1H), 7.74 (d, J=5.8 Hz, 1H), 6.95 (br d, J=8.5 Hz, 1H), 6.42 (dd, J=1.8, 5.7 Hz, 1H), 6.32 (s, 1H), 5.99 (s, 1H), 5.01 (br s, 1H), 4.10-3.91 (m, 2H), 3.71-3.54 (m, 1H), 3.22-3.16 (m, 1H), 2.39 (dt, J=5.4, 10.2 Hz, 1H), 2.12-1.97 (m, 1H), 1.82-1.69 (m, 6H), 1.61-1.43 (m, 2H), 1.08-0.98 (m, 3H) ppm.

Example 4: Synthesis of (1¹S,1³R,Z)-9,9-dimethyl-2¹H-5,12-dioxa-3,10-diaza-4(4,2)-pyridina-2(3,5)-pyrazola-1 (1,3)-cyclopentanacyclododecaphan-11-one (Compound 13)

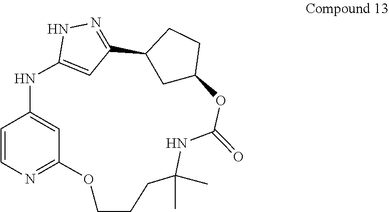 [SMILES: CC1(C)CCCOc2cc(ccn2)Nc2cc(n[nH]2)[C@H]2CC[C@H](C2)OC(=O)N1]

Step 1: Preparation of 4-methyl-4-nitropentan-1-ol

To a mixture of methyl 4-methyl-4-nitropentanoate (500 mg, 2.85 mmol, 1 eq) and LiCl (240 mg, 5.71 mmol, 2 eq) in THF (5 mL) and EtOH (5 mL) was added LiBH₄ (2 M, 2.85 mL, 2 eq) at 0° C. under N₂ atmosphere. The mixture was then warmed to 20° C. and stirred for 4 hours under N₂ atmosphere. After the completion of the reaction, the mixture was quenched with NH₄Cl (aq, 20 mL) at 0° C. and extracted with EtOAc (20 mL×3). The combined organic layers were dried over Na₂SO₄, filtered, and the filtrate was concentrated under reduced pressure to give 4-methyl-4-nitropentan-1-ol (400 mg, 95.23% yield) as a colorless oil. ¹H NMR (400 MHz, METHANOL-d₄) δ 3.54 (t, J=6.4 Hz, 2H), 2.00-1.92 (m, 2H), 1.58 (s, 6H), 1.51-1.42 (m, 2H) ppm.

Step 2: Preparation of 4-amino-4-methyl-pentan-1-ol

A mixture of 4-methyl-4-nitropentan-1-ol (250 mg, 1.70 mmol, 1 eq) and Raney-Ni (25.00 mg, 0.058 mmol, 20% purity) in MeOH (10 mL) was degassed and backfilled with H₂ for 3 times and stirred at 60° C. for 12 hours under H₂ (15 psi) atmosphere. After the completion of the reaction, the reaction mixture was cooled to room temperature. The solid was filtered off, rinsed with MeOH (15 mL) and the filtrate was concentrated to give 4-amino-4-methyl-pentan-1-ol (199 mg, 99.97% yield), which was used into the next step without further purification.

Step 3: Preparation of tert-butyl (5-hydroxy-2-methylpentan-2-yl)carbamate

The mixture of 4-amino-4-methyl-pentan-1-ol (199 mg, 1.70 mmol, 1 eq), NaHCO₃(570.61 mg, 6.79 mmol, 4 eq) and Boc₂O (555.91 mg, 2.55 mmol, 1.5 eq) in THF (5 mL) was stirred at 25° C. for 3 hours under N₂ atmosphere. After the completion of the reaction, the mixture was concentrated under reduced pressure. The resulting residue was diluted with water (10 mL) and extracted with EtOAc (10 mL×3). The combined organic layers were washed with brine (10 mL), dried over Na₂SO₄, filtered, and the filtrate was concentrated under reduced pressure. The desired tert-butyl (5-hydroxy-2-methylpentan-2-yl)carbamate (500 mg, crude) was obtained as a colorless oil, which was used into the next step without further purification. 1H NMR (400 MHz, CHLOROFORM-d) δ 4.58-4.31 (m, 1H), 3.65 (t, J=6.4 Hz, 2H), 1.76-1.69 (m, 2H), 1.62-1.57 (m, 2H), 1.43 (s, 9H), 1.27 (s, 6H) ppm.

Step 4: Preparation of tert-butyl (5-((4-bromopyridin-2-yl)oxy)-2-methylpentan-2-yl)carbamate

To a solution of tert-butyl (5-hydroxy-2-methylpentan-2-yl)carbamate (500 mg, 2.30 mmol, 1 eq) in THF (10 mL) was added 4-bromo-2-fluoro-pyridine (404.93 mg, 2.30 mmol, 1 eq) and t-BuOK (387.29 mg, 3.45 mmol, 1.5 eq) at 0° C., and the mixture was warmed to 25° C. and stirred for 12 hours under N₂ atmosphere. After the completion of the reaction, the mixture was filtered, rinsed with THF (10 mL), and the filtrate was concentrated. The resulting residue was purified by column chromatography to give tert-butyl (5-((4-bromopyridin-2-yl)oxy)-2-methylpentan-2-yl)carbamate (320 mg, 37.26% yield) as a colorless oil. LCMS: 373.2/375.2 [M+H]⁺; ¹H NMR (400 MHz, CHLOROFORM-d) δ 7.98 (d, J=5.5 Hz, 1H), 7.03 (dd, J=1.6, 5.6 Hz, 1H), 6.96 (d, J=1.4 Hz, 1H), 4.39 (br s, 1H), 4.30 (br t, J=5.7 Hz, 2H), 1.78 (br d, J=3.3 Hz, 3H), 1.70-1.66 (m, 4H), 1.49 (s, 9H), 1.29 (s, 3H) ppm.

Step 5: Preparation of tert-butyl (5-((4-((1-(tert-butyl)-3-((1S,3R)-3-((tert-butyldimethylsilyl)oxy)cyclopentyl)-1H-pyrazol-5-yl)amino)pyridin-2-yl)oxy)-2-methylpentan-2-yl)carbamate

A mixture of tert-butyl (5-((4-bromopyridin-2-yl)oxy)-2-methylpentan-2-yl)carbamate (250 mg, 0.67 mmol, 1 eq), 1-(tert-butyl)-3-((1S,3R)-3-((tert-butyldimethylsilyl)oxy)cyclopentyl)-1H-pyrazol-5-amine (226.08 mg, 0.67 mmol, 1 eq), Pd₂(dba)₃ (61.33 mg, 0.067 mmol, 0.1 eq), Xantphos (63.85 mg, 0.134 mmol, 0.2 eq) and Cs₂CO₃ (654.63 mg, 2.01 mmol, 3 eq) in dioxane (6 mL) was heated and stirred at 90° C. for 12 hours under N₂ atmosphere. The reaction mixture was then cooled to room temperature. The mixture was filtered through a short pad of celite, rinsed with dioxane (10 mL) and the filtrate was concentrated. The resulting residue was purified by column chromatography to give tert-butyl (5-((4-((1-(tert-butyl)-3-((1S,3R)-3-((tert-butyldimethylsilyl)oxy)cyclopentyl)-1H-pyrazol-5-yl)amino)pyridin-2-yl)oxy)-2-methylpentan-2-yl)carbamate (190 mg, 45.03% yield) as a yellow oil. LCMS: 630.5 [M+H]⁺; 1H NMR (400 MHz, CHLOROFORM-d) δ 7.84 (d, J=5.9 Hz, 1H), 6.38-6.23 (m, 1H), 6.11-5.93 (m, 2H), 4.52-4.41 (m, 1H), 4.37-4.29 (m, 1H), 4.25 (br s, 2H), 3.11-2.98 (m, 1H), 2.25 (s, 1H), 2.04-1.95 (m, 1H), 1.91-1.79 (m, 3H), 1.76 (br d, J=2.8 Hz, 3H), 1.73-1.68 (m, 2H), 1.68-1.62 (m, 4H), 1.58 (s, 9H), 1.43 (s, 9H), 0.90 (s, 3H), 0.88 (s, 9H), 0.07-0.05 (m, 6H) ppm.

Step 6: Preparation of tert-butyl (2-((4-((tert-butoxycarbonyl)amino)-4-methylpentyl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (5-((4-((1-(tert-butyl)-3-((1S,3R)-3-((tert-butyldimethylsilyl)oxy)cyclopentyl)-1H-pyrazol-5-yl)amino)pyridin-2-yl)oxy)-2-methylpentan-2-yl)carbamate (190 mg, 0.302 mmol, 1 eq) in THF (5 mL) was added Boc₂O (131.65 mg, 0.603 mmol, 138.58 μL, 2 eq), TEA (91.56 mg, 0.905 mmol, 125.94 μL, 3 eq) and DMAP (36.85 mg, 0.302 mmol, 1 eq) at 0° C., and the mixture was stirred at 30° C. for 12 hours under N₂ atmosphere. After the completion of the reaction, the volatiles were removed under reduced pressure. The resulting residue was diluted with water (10 mL) and extracted with EA (10 mL×3). The combined organic layers were dried over Na₂SO₄, filtered, and the filtrate was concentrated under reduced pressure. The resulting crude was purified by column chromatography to give tert-butyl (2-((4-((tert-butoxycarbonyl)amino)-4-methylpentyl)oxy)pyridin-4-yl)(1-(tert-butyl)-3-((1S,3R)-3-((tert-butyldimethylsilyl)oxy)cyclopentyl)-1H-pyrazol-5-yl)carbamate (165 mg, 74.93% yield) as a yellow oil.

Step 7: Preparation of tert-butyl (2-((4-((tert-butoxycarbonyl)amino)-4-methylpentyl)oxy)pyridin-4-yl)(1-(tert-butyl)-3-((1S,3R)-3-hydroxycyclopentyl)-1H-pyrazol-5-yl)carbamate

To a solution of tert-butyl (2-((4-((tert-butoxycarbonyl)amino)-4-methylpentyl)oxy)pyridin-4-yl)(1-(tert-butyl)-3-((1S,3R)-3-((tert-butyldimethylsilyl)oxy)cyclopentyl)-1H-pyrazol-5-yl)carbamate (165 mg, 0.226 mmol, 1 eq) in THF (6 mL) was added TBAF (1 M, 1.13 mL, 5 eq), and the mixture was stirred at 20° C. for 12 hours under N₂ atmosphere. After the completion of the reaction, the volatiles were removed under reduced pressure. The resulting residue was purified by prep-TLC (SiO₂, PE:EA=2:1, R_{f}=0.3) to give tert-butyl (2-((4-((tert-butoxycarbonyl)amino)-4-methylpentyl)oxy)pyridin-4-yl)(1-(tert-butyl)-3-((1S,3R)-3-hydroxycyclopentyl)-1H-pyrazol-5-yl)carbamate (150 mg, crude) as a colorless oil. LCMS: 616.5[M+H]⁺; ¹H NMR (400 MHz, CHLOROFORM-d) δ 8.02 (d, J=5.9 Hz, 1H), 6.84 (br s, 1H), 6.82-6.75 (m, 1H), 5.96 (d, J=2.4 Hz, 1H), 4.49-4.42 (m, 1H), 4.41-4.36 (m, 1H), 4.33-4.23 (m, 2H), 3.37-3.23 (m, 1H), 2.23-2.08 (m, 2H), 2.02-1.89 (m, 2H), 1.62-1.55 (m, 6H), 1.46 (s, 9H), 1.45 (d, J=4.1 Hz, 9H), 1.43 (s, 9H), 1.28 (s, 6H) ppm.

Step 8: Preparation of tert-butyl (2-((4-((tert-butoxycarbonyl)amino)-4-methylpentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((4-((tert-butoxycarbonyl)amino)-4-methylpentyl)oxy)pyridin-4-yl)(1-(tert-butyl)-3-((1S,3R)-3-hydroxycyclopentyl)-1H-pyrazol-5-yl)carbamate (150 mg, 0.244 mmol, 1 eq) in THF (3 mL) was added Py (96.34 mg, 1.22 mmol, 5 eq) and DMAP (14.88 mg, 0.128 mmol, 0.5 eq), followed by a solution of (4-nitrophenyl) chloroformate (245.49 mg, 1.22 mmol, 5 eq) in DCM (3 mL) at 0° C. After the completion of the addition, the mixture was stirred at 30° C. for 12 hours under N₂ atmosphere, and then the volatiles were removed under reduced pressure. The resulting residue was diluted with water (10 mL) and extracted with EtOAc (10 mL×3). The combined organic layer was dried over Na₂SO₄, filtered, and the filtrate was concentrated under reduced pressure. The resulting residue was purified by prep-TLC (SiO₂, PE:EA=2:1, R_{f}=0.4) to give tert-butyl (2-((4-((tert-butoxycarbonyl)amino)-4-methylpentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (130 mg, 68.34% yield) as a yellow oil. LCMS: 781.5 [M+H]⁺.

Step 9: Preparation of (1R,3S)-3-(5-((2-((4-amino-4-methylpentyl)oxy)pyridin-4-yl)amino)-1-(tert-butyl)-1H-pyrazol-3-yl)cyclopentyl (4-nitrophenyl) carbonate

To a solution of tert-butyl (2-((4-((tert-butoxycarbonyl)amino)-4-methylpentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (100 mg, 0.128 mmol, 1 eq) in DCM (3 mL) was added TFA (921.00 mg, 8.08 mmol, 0.6 mL, 63.08 eq). After the mixture was stirred at 30° C. for 3 hours under N₂ atmosphere, the volatiles were removed under reduced pressure. The desired (1R,3S)-3-(5-((2-((4-amino-4-methylpentyl)oxy)pyridin-4-yl)amino)-1-(tert-butyl)-1H-pyrazol-3-yl)cyclopentyl (4-nitrophenyl) carbonate (74 mg, 99.52% yield,) was obtained as a yellow oil. LCMS: 581.4 [M+H]⁺.

Step 10: Preparation of (1¹S,1³R,Z)-2¹-(tert-butyl)-9,9-dimethyl-2¹H-5,12-dioxa-3,10-diaza-4(4,2)-pyridina-2(3,5)-pyrazola-1(1,3)-cyclopentanacyclododecaphan-11-one

To a solution of (1R,3S)-3-(5-((2-((4-amino-4-methylpentyl)oxy)pyridin-4-yl)amino)-1-(tert-butyl)-1H-pyrazol-3-yl)cyclopentyl (4-nitrophenyl) carbonate (74 mg, 0.127 mmol, 1 eq) in THF (70 mL) was added DIPEA (82.35 mg, 0.637 mmol, 110.99 μL, 5 eq) and DMAP (15.57 mg, 0.127 mmol, 1 eq), and the mixture was first stirred at 30° C. for 3 hours, and then at 50° C. for 12 hours under N₂ atmosphere. An additional DMAP (25 mg, 0.205 mmol, 1.61 eq) was then added, and the mixture was kept stirring at 50° C. for an additional 24 hours under N₂ atmosphere. The mixture was cooled to room temperature and concentrated under reduced pressure. The resulting residue was purified by prep-TLC (SiO₂, DCM:MeOH=10:1, R_{f}=0.4) to give (1¹S,1³R,Z)-2¹-(tert-butyl)-9,9-dimethyl-2¹H-5,12-dioxa-3,10-diaza-4(4,2)-pyridina-2(3,5)-pyrazola-1(1,3)-cyclopentanacyclododecaphan-11-one (50 mg, 88.85% yield) as a yellow oil. LCMS: 442.6 [M+H]⁺.

Step 11: Preparation of (1¹S,1³R,Z)-9,9-dimethyl-2¹H-5,12-dioxa-3,10-diaza-4(4,2)-pyridina-2(3,5)-pyrazola-1(1,3)-cyclopentanacyclododecaphan-11-one

A mixture of (1¹S,1³R,Z)-2¹-(tert-butyl)-9,9-dimethyl-2¹H-5,12-dioxa-3,10-diaza-4(4,2)-pyridina-2(3,5)-pyrazola-1(1,3)-cyclopentanacyclododecaphan-11-one (50 mg, 0.113 mmol, 1 eq) in formic acid (3 mL) was stirred at 100° C. for 0.5 hour under N₂ atmosphere. After the completion of the reaction, the reaction mixture was cooled to room temperature, and the volatiles were removed under reduced pressure. The resulting residue was purified by prep-HPLC (column: Phenomenex Luna C18 150×25 mm, 10 um; mobile phase: [water (FA)-ACN]; gradient:12%-42% B over 7 min) to give (1¹S,1³R,Z)-9,9-dimethyl-2¹H-5,12-dioxa-3,10-diaza-4(4,2)-pyridina-2(3,5)-pyrazola-1 (1,3)-cyclopentanacyclododecaphan-11-one (1.97 mg, 3.77% yield) as a white solid. LCMS: 386.2 [M+H]⁺; ¹H NMR (400 MHz, DMSO-d₆) δ 12.08 (br s, 1H), 8.96 (s, 1H), 8.35 (s, 1H), 7.73 (d, J=5.8 Hz, 1H), 6.49 (s, 1H), 6.43 (dd, J=1.9, 5.8 Hz, 1H), 6.37 (d, J=1.6 Hz, 1H), 6.05 (s, 1H), 5.08-4.98 (m, 1H), 4.13-4.04 (m, 1H), 3.98-3.88 (m, 1H), 2.43-2.37 (m, 1H), 1.97 (br dd, J=5.1, 6.7 Hz, 1H), 1.89 (br d, J=6.8 Hz, 1H), 1.87-1.83 (m, 2H), 1.82-1.77 (m, 2H), 1.70-1.64 (m, 1H), 1.55-1.48 (m, 1H), 1.30 (br s, 3H), 1.25-1.21 (m, 2H), 1.15 (s, 3H) ppm.

Example 5: Synthesis of (1¹S,1³R,Z)-4⁶-(difluoromethyl)-2¹H-12-oxa-3,10-diaza-4(4,2)-pyridina-2(3,5)-pyrazola-1(1,3)-cyclopentanacyclododecaphan-11-one (Compound 17)

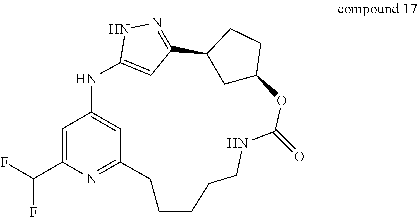 [SMILES: O=C1NCCCCCc2cc(cc(C(F)F)n2)Nc2cc(n[nH]2)[C@H]2CC[C@H](C2)O1]

Step 1: Preparation of (4,6-dichloropyridin-2-yl)methanol

To a solution of methyl 4,6-dichloropicolinate (5 g, 24.27 mmol, 1 eq) in MeOH (60 mL) was added NaBH₄ (1.84 g, 48.54 mmol, 2 eq) at 0° C., and the mixture was stirred at 20° C. for 2 hours under N₂ atmosphere. The reaction mixture was then quenched with saturated aqueous NH₄Cl (200 mL) at 0° C. and extracted with EtOAc (200 mL×3). The combined organic layers were washed with brine (100 mL), dried over Na₂SO₄, filtered, and the filtrate was concentrated under reduced pressure. The resulting residue was purified by column chromatography to give (4,6-dichloropyridin-2-yl)methanol (4.3 g, 99.53% yield) as a white solid. LCMS: 178.1 [M+H]⁺; ¹H NMR (400 MHz, CHLOROFORM-d) δ 7.32 (d, J=0.6 Hz, 1H), 7.30-7.28 (m, 1H), 4.75 (s, 2H), 3.01-2.55 (m, 1H) ppm.

Step 2: Preparation of 4,6-dichloropicolinaldehyde

To a mixture of (4,6-dichloropyridin-2-yl)methanol (3.3 g, 18.54 mmol, 1 eq) in DCM (60 mL) was added DMP (15.73 g, 37.08 mmol, 11.49 mL, 2 eq) in one portion at 0° C., and the mixture was stirred at 25° C. for 2 hours under N₂ atmosphere. The reaction mixture was then quenched with saturated aqueous Na₂S203 (100 mL) at 0° C. and extracted with DCM (100 mL×3). The combined organic layers were washed with brine (100 mL), dried over Na₂SO₄, filtered, and the filtrate was concentrated under reduced pressure. The resulting residue was purified by column chromatography to give 4,6-dichloropicolinaldehyde (2.9 g, 88.89% yield) as a white solid. LCMS: 176.0 [M+H]⁺; ¹H NMR (400 MHz, CHLOROFORM-d) δ 9.97 (s, 1H) 7.87 (s, 1H) 7.60 (s, 1H) ppm.

Step 3: Preparation of 2,4-dichloro-6-(difluoromethyl)pyridine

To a mixture of 4,6-dichloropicolinaldehyde (2.9 g, 16.48 mmol, 1 eq) in DCM (30 mL) was added DAST (5.31 g, 32.95 mmol, 4.35 mL, 2 eq) in one portion at −20° C. under N₂ atmosphere. The mixture was then warmed to 25° C. and kept stirring for 2 hours under N₂ atmosphere. After the starting material was consumed, the mixture was diluted with H₂O (50 mL) and extracted with ethyl acetate (30 mL×3). The combined organic layers were washed with brine (30 mL), dried over Na₂SO₄, filtered, and the filtrate was concentrated under reduced pressure. The resulting residue was purified by column chromatography to give 2,4-dichloro-6-(difluoromethyl)pyridine (2 g, 61.30% yield) as a yellow oil. LCMS: 198.0 [M+H]⁺; ¹H NMR (400 MHz, CHLOROFORM-d) δ 7.59 (d, J=1.25 Hz, 1H) 7.49 (d, J=0.63 Hz, 1H), 6.56 (t, J=54.8 Hz, 1H) ppm.

Step 4: Preparation of tert-butyl (5-(4-chloro-6-(difluoromethyl)pyridin-2-yl)pent-4-yn-1-yl)carbamate

The mixture of 2,4-dichloro-6-(difluoromethyl)pyridine (1 g, 5.05 mmol, 1 eq), tert-butyl pent-4-yn-1-ylcarbamate (832.95 mg, 4.55 mmol, 0.9 eq), CuI (192.38 mg, 1.01 mmol, 0.2 eq), TEA (1.02 g, 10.10 mmol, 1.41 mL, 2 eq) and Pd(PPh₃)₂Cl₂ (354.50 mg, 0.505 mmol, 0.1 eq) in DMF (10 mL) was stirred at 25° C. for 12 hours under N₂ atmosphere. After the starting materials were consumed, the reaction mixture was diluted with H₂O (50 mL) and extracted with ethyl acetate (30 mL×3). The combined organic layers were washed with brine (30 mL), dried over Na₂SO₄, filtered, and the filtrate was concentrated under reduced pressure. The resulting residue was purified by column chromatography to give tert-butyl (5-(4-chloro-6-(difluoromethyl)pyridin-2-yl)pent-4-yn-1-yl)carbamate (910 mg, 52.26% yield) as a yellow oil. LCMS: 345.3 [M+H]⁺.

Step 5: Preparation of tert-butyl (5-(4-chloro-6-(difluoromethyl)pyridin-2-yl)pentyl)carbamate

The mixture of tert-butyl (5-(4-chloro-6-(difluoromethyl)pyridin-2-yl)pent-4-yn-1-yl)carbamate (950 mg, 2.76 mmol, 1 eq) and Pt/C (95.00 mg, 0.0226 mmol, 5% purity) in THF (10 mL) was degassed, backfilled with H₂ for 3 times and stirred at 30° C. for 4 hours under H₂ (15 psi) atmosphere. After the completion of the reaction, the mixture was filtered through a short pad of celite, rinsed with THF (10 mL) and the filtrate was concentrated under reduced pressure. The resulting residue was purified by column chromatography to give tert-butyl (5-(4-chloro-6-(difluoromethyl)pyridin-2-yl)pentyl)carbamate (430 mg, 44.74% yield) as a yellow oil. LCMS: 349.2 [M+H]⁺.

Step 6: Preparation of tert-butyl (5-(4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)pentyl)carbamate

The mixture of tert-butyl (5-(4-chloro-6-(difluoromethyl)pyridin-2-yl)pentyl)carbamate (210 mg, 0.602 mmol, 1 eq), 1-(tert-butyl)-3-((1S,3R)-3-((tert-butyldimethylsilyl)oxy)cyclopentyl)-1H-pyrazol-5-amine (203.23 mg, 0.602 mmol, 1 eq), Cs₂CO₃ (588.47 mg, 1.81 mmol, 3 eq), Xantphos (57.40 mg, 0.120 mmol, 0.2 eq) and Pd₂(dba)₃ (55.13 mg, 0.0602 mmol, 0.1 eq) in dioxane (4 mL) was heated and stirred at 90° C. for 12 hours under N₂ atmosphere. After the completion of the reaction, the reaction mixture was cooled to room temperature, filtered through a short pad of celite, rinsed with dioxane (10 mL) and the filtrate was concentrated under reduced pressure. The resulting residue was purified by prep-TLC (petroleum ether/ethyl acetate=4/1) to give tert-butyl (5-(4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)pentyl)carbamate (182 mg, 46.51% yield) as a yellow oil. LCMS: 650.5 [M+H]⁺; ¹H NMR (400 MHz, DMSO-d₆) δ 6.85-6.55 (m, 3H), 6.46 (s, 1H), 6.00 (s, 1H), 4.31 (quin, J=5.13 Hz, 1H), 3.00 (quin, J=8.35 Hz, 1H), 2.87 (q, J=6.59 Hz, 2H), 2.55 (br t, J=7.69 Hz, 2H), 2.41-1.86 (m, 3H), 1.82-1.72 (m, 2H), 1.62-1.52 (m, 4H), 1.48 (s, 9H), 1.36 (s, 9H), 1.27-1.16 (m, 4H), 0.82 (s, 9H), 0.02 (d, J=4.50 Hz, 6H) ppm.

Step 7: Preparation of tert-butyl (2-(5-((tert-butoxycarbonyl)amino)pentyl)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (5-(4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)pentyl)carbamate (140 mg, 0.215 mmol, 1 eq) in THF (3 mL) was added Boc₂O (94.02 mg, 0.431 mmol, 98.97 μL, 2 eq), TEA (65.39 mg, 0.646 mmol, 89.95 μL, 3 eq) and DMAP (26.32 mg, 0.215 mmol, 1 eq) at 0° C., and the mixture was stirred at 25° C. for 12 hours under N₂ atmosphere. After the completion of the reaction, the volatiles were removed under reduced pressure. The resulting residue was purified by prep-TLC (petroleum ether/ethyl acetate=5/1) to give tert-butyl (2-(5-((tert-butoxycarbonyl)amino)pentyl)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate (52 mg, 32.19% yield) as a yellow oil. LCMS: 750.5 [M+H]⁺.

Step 8: Preparation of tert-butyl (2-(5-((tert-butoxycarbonyl)amino)pentyl)-6-(difluoromethyl)pyridin-4-yl)(1-(tert-butyl)-3-((1S,3R)-3-hydroxycyclopentyl)-1H-pyrazol-5-yl)carbamate

The solution of tert-butyl (2-(5-((tert-butoxycarbonyl)amino)pentyl)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate (52 mg, 0.0693 mmol, 1 eq) and TBAF (1 M, 346.65 μL, 5 eq) in THF (1.75 mL) was stirred at 25° C. for 12 hours under N₂ atmosphere. After the completion of the reaction, the volatiles were removed under reduced pressure. The resulting residue was purified by prep-TLC (petroleum ether/ethyl acetate=5/1) to give tert-butyl (2-(5-((tert-butoxycarbonyl)amino)pentyl)-6-(difluoromethyl)pyridin-4-yl)(1-(tert-butyl)-3-((1S,3R)-3-hydroxycyclopentyl)-1H-pyrazol-5-yl)carbamate (49 mg, crude) as a yellow oil. LCMS: 636.4 [M+H]⁺.

Step 9: Preparation of tert-butyl (2-(5-((tert-butoxycarbonyl)amino)pentyl)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(5-((tert-butoxycarbonyl)amino)pentyl)-6-(difluoromethyl)pyridin-4-yl)(1-(tert-butyl)-3-((1S,3R)-3-hydroxycyclopentyl)-1H-pyrazol-5-yl)carbamate (49 mg, 0.0771 mmol, 1 eq) in THF (1 mL) was added Py (36.58 mg, 0.462 mmol, 37.32 μL, 6 eq) and DMAP (4.71 mg, 0.0385 mmol, 0.5 eq), followed by addition of a solution of (4-nitrophenyl)chloroformate (77.67 mg, 0.385 mmol, 5 eq) in DCM (1 mL) at 0° C. under N₂ atmosphere. After the completion of the addition, the reaction was kept stirring at 30° C. for 12 hours under N₂ atmosphere. The reaction mixture was then filtered and the filtrate was concentrated under reduced pressure. The resulting residue was diluted with H₂O (5 mL) and extracted with ethyl acetate (5 mL×3). The combined organic layers were washed with brine (5 mL), dried over Na₂SO₄, filtered, and the filtrate was concentrated under reduced pressure. The resulting residue was purified by prep-TLC (petroleum ether/ethyl acetate=3/1) to give tert-butyl (2-(5-((tert-butoxycarbonyl)amino)pentyl)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate (38 mg, 61.56% yield) as a yellow oil. LCMS: 801.8 [M+H]⁺.

Step 10: Preparation of (1R,3S)-3-(5-((2-(5-aminopentyl)-6-(difluoromethyl)pyridin-4-yl)amino)-1-(tert-butyl)-1H-pyrazol-3-yl)cyclopentyl (4-nitrophenyl) carbonate

The solution of tert-butyl (2-(5-((tert-butoxycarbonyl)amino)pentyl)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate (38 mg, 0.047 mmol, 1 eq) and TFA (307.00 mg, 2.69 mmol, 0.2 mL, 56.75 eq) in DCM (1 mL) was stirred at 30° C. for 12 hours under N₂ atmosphere and then the volatiles were removed under reduced pressure. The desired (1R,3S)-3-(5-((2-(5-aminopentyl)-6-(difluoromethyl)pyridin-4-yl)amino)-1-(tert-butyl)-1H-pyrazol-3-yl)cyclopentyl (4-nitrophenyl) carbonate (59 mg, crude) was obtained as a yellow oil. LCMS: 601.2 [M+H]⁺.

Step 11: Preparation of (1¹S,1³R, Z)-2¹-(tert-butyl)-4⁶-(difluoromethyl)-2¹H-12-oxa-3,10-diaza-4(4,2)-pyridina-2(3,5)-pyrazola-1(1,3)-cyclopentanacyclododecaphan-11-one

To a solution of (1R,3S)-3-(5-((2-(5-aminopentyl)-6-(difluoromethyl)pyridin-4-yl)amino)-1-(tert-butyl)-1H-pyrazol-3-yl)cyclopentyl (4-nitrophenyl) carbonate (59 mg, 0.0982 mmol, 1 eq) in THF (30 mL) was added DIPEA (88.87 mg, 0.688 mmol, 119.76 μL, 7 eq), and the mixture was stirred at 30° C. for 12 hours under N₂ atmosphere. The volatiles were then removed under reduced pressure to give a residue, which was purified by prep-TLC (petroleum ether/ethyl acetate=1/1) to give (1¹S,1³R, Z)-2¹-(tert-butyl)-4⁶-(difluoromethyl)-2¹H-12-oxa-3,10-diaza-4(4,2)-pyridina-2(3,5)-pyrazola-1(1,3)-cyclopentanacyclododecaphan-11-one (20 mg, 44.12% yield) as a yellow oil. LCMS: 462.3 [M+H]⁺.

Step 12: Preparation of (1¹S,1³R,Z)-4⁶-(difluoromethyl)-2¹H-12-oxa-3,10-diaza-4(4,2)-pyridina-2(3,5)-pyrazola-1(1,3)-cyclopentanacyclododecaphan-11-one

A solution of (1¹S,1³R, Z)-2¹-(tert-butyl)-4⁶-(difluoromethyl)-2¹H-12-oxa-3,10-diaza-4(4,2)-pyridina-2(3,5)-pyrazola-1(1,3)-cyclopentanacyclododecaphan-11-one (20 mg, 0.0433 mmol, 1 eq) in HCOOH (1 mL) was stirred at 100° C. for 0.5 hour under N₂ atmosphere. After the completion of the reaction, the reaction mixture was cooled to room temperature and concentrated under reduced pressure. The resulting residue was purified by pre-HPLC (column: YMC-Actus Triart C18 150×30 mm, 7 um; mobile phase: [water (FA)-ACN]; gradient:10%-40% B over 10 min to give (1¹S,1³R,Z)-4⁶-(difluoromethyl)-2¹H-12-oxa-3,10-diaza-4(4,2)-pyridina-2(3,5)-pyrazola-1(1,3)-cyclopentanacyclododecaphan-11-one (9.8 mg, 54.67% yield) as a white solid. LCMS: 406.2 [M+H]⁺; ¹H NMR (400 MHz, DMSO-d₆) δ 12.37-12.06 (m, 1H), 9.20-9.07 (m, 1H), 7.10 (dd, J=4.8, 2.8 Hz, 1H), 6.89 (s, 1H), 6.85-6.55 (m, 2H), 5.97 (s, 1H), 5.13-4.93 (m, 1H), 3.02-2.86 (m, 1H), 2.81-2.70 (m, 1H), 2.62 (d, J=6.4 Hz, 2H), 2.48-2.34 (m, 2H), 2.11-1.98 (m, 1H), 1.86-1.58 (m, 6H), 1.50-1.22 (m, 4H) ppm.

Example 6: Synthesis of (1¹S,1³R,Z)-2¹H-5,10-dioxa-3,8-diaza-4(4,2)-pyridina-2(3,5)-pyrazola-7(1,3)-bicyclo[1.1.1]pentana-1(1,3)-cyclopentanacyclodecaphan-9-one (Compound 18)

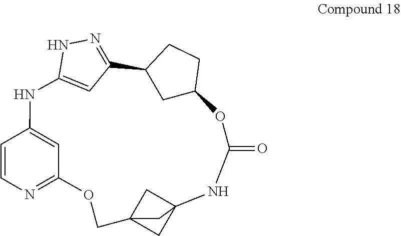 [SMILES: O=C1NC23CC(COc4cc(ccn4)Nc4cc(n[nH]4)[C@H]4CC[C@H](C4)O1)(C2)C3]

Step 1: Preparation of tert-butyl (3-(hydroxymethyl)bicyclo[1.1.1]pentan-1-yl)carbamate

To a solution of methyl 3-((tert-butoxycarbonyl)amino)bicyclo[1.1.1]pentane-1-carboxylate (4 g, 16.58 mmol, 1 eq) in THF (80 mL) was added LiAlH₄ (1.26 g, 33.16 mmol, 2 eq), and the mixture was stirred at 20° C. for 2 hours under N₂ atmosphere. After the completion of the reaction, the reaction was subsequently quenched with H₂O (20 mL), 15% NaOH (20 mL), H₂O (60 mL), and extracted with EtOAc (100 mL×2). The combined organic layers were washed with brine (50 mL), dried over Na₂SO₄, filtered, and the filtrate was concentrated under reduced pressure to give tert-butyl (3-(hydroxymethyl)bicyclo[1.1.1]pentan-1-yl)carbamate (3.5 g, crude) as a white solid. 1H NMR (400 MHz, DMSO-d₆) δ 7.39 (br s, 1H), 4.45 (t, J=5.2 Hz, 1H), 3.42 (d, J=5.2 Hz, 2H), 1.73 (s, 6H), 1.36 (s, 9H) ppm.

Step 2: Preparation of tert-butyl (3-(((4-bromopyridin-2-yl)oxy)methyl)bicyclo[1.1.1]pentan-1-yl)carbamate

To a solution of tert-butyl (3-(hydroxymethyl)bicyclo[1.1.1]pentan-1-yl)carbamate (500 mg, 2.34 mmol, 1 eq), 4-bromopyridin-2-ol (448.71 mg, 2.58 mmol, 1.1 eq) and PPh₃ (737.89 mg, 2.81 mmol, 1.2 eq) in THF (10 mL), DIAD (568.87 mg, 2.81 mmol, 545.42 μL, 1.2 eq) was added at 0° C. under N₂ atmosphere. The mixture was stirred at 20° C. for 2 hours under N₂ atmosphere and concentrated under reduced pressure. The resulting residue was diluted with H₂O (100 mL) and extracted with EtOAc (100 mL×2). The combined organic layers were washed with brine (50 mL), dried over Na₂SO₄, filtered and the filtrate was concentrated. The resulting residue was purified by column chromatography to give tert-butyl (3-(((4-bromopyridin-2-yl)oxy)methyl)bicyclo[1.1.1]pentan-1-yl)carbamate (550 mg, 63.53% yield) as a white solid. LCMS: 369.1 [M+H]⁺.

Step 3: Preparation of tert-butyl (3-(((4-((1-(tert-butyl)-3-((1S,3R)-3-((tert-butyldimethylsilyl)oxy)cyclopentyl)-1H-pyrazol-5-yl)amino)pyridin-2-yl)oxy)methyl)bicyclo[1.1.1]pentan-1-yl)carbamate

A mixture of tert-butyl (3-(((4-bromopyridin-2-yl)oxy)methyl)bicyclo[1.1.1]pentan-1-yl)carbamate (540 mg, 1.46 mmol, 1 eq), 1-(tert-butyl)-3-((1S,3R)-3-((tert-butyldimethylsilyl)oxy)cyclopentyl)-1H-pyrazol-5-amine (740.51 mg, 2.19 mmol, 1.5 eq), Xantphos (169.24 mg, 0.292 mmol, 0.2 eq), Pd₂(dba)₃ (133.92 mg, 0.146 mmol, 0.1 eq) and Cs₂CO₃ (1.43 g, 4.39 mmol, 3 eq) in dioxane (20 mL) was degassed, backfilled with N₂ for 3 times, and then heated and stirred at 90° C. for 12 hours under N₂ atmosphere. After the completion of the reaction, the reaction mixture was cooled to room temperature, filtered through a short pad of celite, rinsed with dioxane (10 mL) and the filtrate was concentrated under reduced pressure. The resulting residue was purified by column chromatography to give tert-butyl (3-(((4-((1-(tert-butyl)-3-((1S,3R)-3-((tert-butyldimethylsilyl)oxy)cyclopentyl)-1H-pyrazol-5-yl)amino)pyridin-2-yl)oxy)methyl)bicyclo[1.1.1]pentan-1-yl)carbamate (910 mg, 1.45 mmol, 99.42% yield) as a yellow solid. LCMS: 626.6 [M+H]⁺.

Step 4: Preparation of tert-butyl (2-((3-((tert-butoxycarbonyl)amino)bicyclo[1.1.1]pentan-1-yl)methoxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (3-(((4-((1-(tert-butyl)-3-((1S,3R)-3-((tert-butyldimethylsilyl)oxy)cyclopentyl)-1H-pyrazol-5-yl)amino)pyridin-2-yl)oxy)methyl)bicyclo[1.1.1]pentan-1-yl)carbamate (900 mg, 1.44 mmol, 1 eq) in DCM (20 mL) was added DMAP (17.57 mg, 0.144 mmol, 0.1 eq) and Boc₂O (376.58 mg, 1.73 mmol, 1.2 eq) at 0° C., and the mixture was stirred at 20° C. for 2 hours under N₂ atmosphere. After the completion of the reaction, the volatiles were removed under reduced pressure. The resulting residue was purified by column chromatography to give tert-butyl (2-((3-((tert-butoxycarbonyl)amino)bicyclo[1.1.1]pentan-1-yl)methoxy)pyridin-4-yl)(1-(tert-butyl)-3-((1S,3R)-3-((tert-butyldimethylsilyl)oxy)cyclopentyl)-1H-pyrazol-5-yl)carbamate (932 mg, 89.28% yield) as a yellow oil. LCMS: 727.0 [M+H]⁺.

Step 5: Preparation of tert-butyl (2-((3-((tert-butoxycarbonyl)amino)bicyclo[1.1.1]pentan-1-yl)methoxy)pyridin-4-yl)(1-(tert-butyl)-3-((1S,3R)-3-hydroxycyclopentyl)-1H-pyrazol-5-yl)carbamate

The solution of tert-butyl (2-((3-((tert-butoxycarbonyl)amino)bicyclo[1.1.1]pentan-1-yl)methoxy)pyridin-4-yl)(1-(tert-butyl)-3-((1S,3R)-3-((tert-butyldimethylsilyl)oxy)cyclopentyl)-1H-pyrazol-5-yl)carbamate (922 mg, 1.27 mmol, 1 eq) and TBAF (1.33 g, 5.08 mmol, 4 eq) in THF (15 mL) was stirred at 20° C. for 12 hours under N₂ atmosphere, and then the volatiles were removed under reduced pressure. The remaining residue was diluted with H₂O (100 mL) and extracted with EtOAc (100 mL×2). The combined organic layers were washed with brine (50 mL), dried over Na₂SO₄, filtered, and the filtrate was concentrated under reduced pressure to give tert-butyl (2-((3-((tert-butoxycarbonyl)amino)bicyclo[1.1.1]pentan-1-yl)methoxy)pyridin-4-yl)(1-(tert-butyl)-3-((1S,3R)-3-hydroxycyclopentyl)-1H-pyrazol-5-yl)carbamate (810 mg, crude) as a yellow oil. LCMS: 612.3 [M+H]⁺.

Step 6: Preparation of tert-butyl (2-((3-((tert-butoxycarbonyl)amino)bicyclo[1.1.1]pentan-1-yl)methoxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((3-((tert-butoxycarbonyl)amino)bicyclo[1.1.1]pentan-1-yl)methoxy)pyridin-4-yl)(1-(tert-butyl)-3-((1S,3R)-3-hydroxycyclopentyl)-1H-pyrazol-5-yl)carbamate (300 mg, 0.490 mmol, 1 eq) in DCM (6 mL) was added 4-nitrophenyl carbonochloridate (148.26 mg, 0.736 mmol, 1.5 eq), Py (116.37 mg, 1.47 mmol, 118.74 μL, 3 eq) and DMAP (5.99 mg, 0.049 mmol, 0.1 eq), and the mixture was stirred at 20° C. for 12 hours under N₂ atmosphere. After the completion of the reaction, the volatiles were removed under reduced pressure. The resulting residue was purified by prep-HPLC (neutral condition) to give tert-butyl (2-((3-((tert-butoxycarbonyl)amino)bicyclo[1.1.1]pentan-1-yl)methoxy)pyridin-4-yl)(1-(tert-butyl)-3-((1S,3R)-3-(((4-nitrophenoxy)carbonyl)oxy)cyclopentyl)-1H-pyrazol-5-yl)carbamate (200 mg, 52.50% yield) as a white solid. LCMS: 777.6 [M+H]⁺.

Step 7: Preparation of (1R,3S)-3-(5-((2-((3-aminobicyclo[1.1.1]pentan-1-yl)methoxy)pyridin-4-yl)amino)-1-(tert-butyl)-1H-pyrazol-3-yl)cyclopentyl (4-nitrophenyl) carbonate

The solution of tert-butyl (2-((3-((tert-butoxycarbonyl)amino)bicyclo[1.1.1]pentan-1-yl)methoxy)pyridin-4-yl)(1-(tert-butyl)-3-((1S,3R)-3-(((4-nitrophenoxy)carbonyl)oxy)cyclopentyl)-1H-pyrazol-5-yl)carbamate (80 mg, 0.103 mmol, 1 eq) and TFA (1 mL) in DCM (3 mL) was stirred at 20° C. for 12 hours under N₂ atmosphere and then the volatiles were removed under reduced pressure. The desired (1R,3S)-3-(5-((2-((3-aminobicyclo[1.1.1]pentan-1-yl)methoxy)pyridin-4-yl)amino)-1-(tert-butyl)-1H-pyrazol-3-yl)cyclopentyl (4-nitrophenyl) carbonate (155 mg, crude) was obtained as a yellow oil. LCMS: 577.3 [M+H]⁺.

Step 8: Preparation of (1¹S,1³R,Z)-2¹-(tert-butyl)-2¹H-5,10-dioxa-3,8-diaza-4(4,2)-pyridina-2(3,5)-pyrazola-7(1,3)-bicyclo[1.1.1]pentana-1(1,3)-cyclopentanacyclodecaphan-9-one

The solution of (1R,3S)-3-(5-((2-((3-aminobicyclo[1.1.1]pentan-1-yl)methoxy)pyridin-4-yl)amino)-1-(tert-butyl)-1H-pyrazol-3-yl)cyclopentyl (4-nitrophenyl) carbonate (300 mg, 0.520 mmol, 1 eq) and DIEA (672.39 mg, 5.20 mmol, 906.19 μL, 10 eq) in DMSO (150 mL) was heated and stirred at 100° C. for 2 hours under N₂ atmosphere. After the completion of the reaction, the mixture was cooled to room temperature, and the volatiles were removed under reduced pressure. The resulting residue was diluted with H₂O (200 mL) and extracted with EtOAc (200 mL×2). The combined organic layers were washed with brine (50 mL), dried over Na₂SO₄, filtered, and the filtrate was concentrated under reduced pressure. The desired (1¹S,1³R,Z)-2¹-(tert-butyl)-2¹H-5,10-dioxa-3,8-diaza-4(4,2)-pyridina-2(3,5)-pyrazola-7(1,3)-bicyclo[1.1 0.1]pentana-1(1,3)-cyclopentanacyclodecaphan-9-one (210 mg, crude) was obtained as a yellow oil. LCMS: 438.3 [M+H]⁺.

Step 9: Preparation of (1¹S,1³R,Z)-2¹H-5,10-dioxa-3,8-diaza-4(4,2)-pyridina-2(3,5)-pyrazola-7(1,3)-bicyclo[1 0.1 0.1]pentana-1(1,3)-cyclopentanacyclodecaphan-9-one

A solution of (1¹S,1³R,Z)-2¹-(tert-butyl)-2¹H-5,10-dioxa-3,8-diaza-4(4,2)-pyridina-2(3,5)-pyrazola-7(1,3)-bicyclo[1.1.1]pentana-1(1,3)-cyclopentanacyclodecaphan-9-one (200 mg, 0.457 mmol, 1 eq) in formic acid (4 mL) was stirred at 100° C. for 0.5 hour under N₂ atmosphere. After the completion of the conversion, the mixture was cooled to room temperature, and the volatiles were evaporated under reduced pressure. The crude product was purified by Prep-HPLC (column: Phenomenex Luna C18 150×25 mm, 10 um; mobile phase: [water (NH₃·H₂O)-ACN]; B %: 10%-90%, 20 min) to give (1¹S,1³R,Z)-2¹H-5,10-dioxa-3,8-diaza-4(4,2)-pyridina-2(3,5)-pyrazola-7(1,3)-bicyclo[1.1.1]pentana-1(1,3)-cyclopentanacyclodecaphan-9-one (49.28 mg, 26.88% yield) as a white solid. LCMS: 382.1 [M+H]⁺; ¹H NMR (400 MHz, DMSO-d₆) δ 12.21 (br s, 1H), 8.58 (s, 1H), 7.75 (d, J=5.6 Hz, 1H), 7.50 (s, 1H), 6.43 (dd, J=5.6, 1.6 Hz, 1H), 6.14 (s, 1H), 5.94 (s, 1H), 5.12 (d, J=5.6 Hz, 1H), 4.20-4.06 (m, 2H), 3.28-3.15 (m, 1H), 2.48-2.39 (m, 1H), 2.14-2.03 (m, 1H), 1.86-1.80 (m, 8H), 1.79-1.71 (m, 2H) ppm.

Example 7: Synthesis of (1¹S,1³R,Z)-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one (Compound 22)

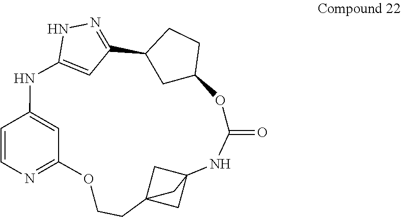 [SMILES: O=C1NC23CC(CCOc4cc(ccn4)Nc4cc(n[nH]4)[C@H]4CC[C@H](C4)O1)(C2)C3]

Step 1: Preparation of (3-((tert-butoxycarbonyl)amino)bicyclo[1.1.1]pentan-1-yl)methyl methanesulfonate

To a solution of tert-butyl (3-(hydroxymethyl)bicyclo[1.1.1]pentan-1-yl)carbamate (2.2 g, 10.32 mmol, 1 eq) in DCM (30 mL) was added TEA (2.09 g, 20.63 mmol, 2.87 mL, 2 eq) and methylsulfonyl methanesulfonate (2.16 g, 12.38 mmol, 1.2 eq) at 0° C., and the mixture was stirred at 0° C. for 0.5 hour under N₂ atmosphere. After the completion of the reaction, the reaction mixture was quenched with NaHCO₃(50 mL). The organic phase was collected, and the aqueous phase was extracted with DCM (40 mL×2). The combined organic layers were washed with brine (70 mL), dried over Na₂SO₄, filtered, and the filtrate was concentrated under reduced pressure. The desired (3-((tert-butoxycarbonyl)amino)bicyclo[1.1.1]pentan-1-yl)methyl methanesulfonate (3.1 g, crude) was obtained as white solid. ¹H NMR (400 MHz, CHLOROFORM-d) δ 5.16-4.79 (m, 1H), 4.30 (s, 2H), 3.01 (s, 3H), 2.04 (s, 6H), 1.44 (s, 9H) ppm.

Step 2: Preparation of tert-butyl (3-(cyanomethyl)bicyclo[1.1.1]pentan-1-yl)carbamate

The solution of (3-((tert-butoxycarbonyl)amino)bicyclo[1.1.1]pentan-1-yl)methyl methanesulfonate (3.1 g, 10.64 mmol, 1 eq) and KCN (1.34 g, 20.58 mmol, 1.93 eq) in DMF (30 mL) was stirred at 70° C. for 24 hours under N₂ atmosphere. After the completion of the reaction, the reaction mixture was cooled to room temperature and diluted with water (80 mL). The aqueous phase was extracted with ethyl acetate (80 mL×3). The combined organic layers were washed with brine (150 mL), dried over Na₂SO₄, filtered, and the filtrate was concentrated under reduced pressure. The desired tert-butyl (3-(cyanomethyl)bicyclo[1.1.1]pentan-1-yl)carbamate (2.1 g, 88.79% yield) was obtained as a yellow solid, which was used in the next step without further purification. ¹H NMR (400 MHz, CHLOROFORM-d) δ 5.00 (br s, 1H), 2.66 (s, 2H), 2.07 (br s, 6H), 1.45 (s, 9H) ppm.

Step 3: Preparation of 2-(3-((tert-butoxycarbonyl)amino)bicyclo[1.1.1]pentan-1-yl)acetic acid

To a solution of tert-butyl (3-(cyanomethyl)bicyclo[1.1.1]pentan-1-yl)carbamate (600 mg, 2.70 mmol, 1 eq) in DCM (10 mL) was added dropwise DIBAL-H (1 M, 8.10 mL, 3 eq) at −78° C. under N₂ atmosphere. After the completion of the addition, the mixture was kept stirring at −78° C. for 2 hours under N₂ atmosphere. The reaction was then carefully quenched with H₂O (100 mL), adjusted pH<3 with HCl (6 M). The reaction mixture was filtrated, rinsed with DCM (50 mL) and the filtrate was extracted with DCM (100 mL×2). The combined organic layers were washed with brine (200 mL), dried over Na₂SO₄, filtered and the filtrate was concentrated under reduced pressure. The desired 2-(3-((tert-butoxycarbonyl)amino)bicyclo[1.1.1]pentan-1-yl)acetic acid (550 mg, 2.28 mmol, 84.45% yield) was obtained as a colorless oil, which was used into the next step without further purification. 1H NMR (400 MHz, METHANOL-d₄) δ 2.08-1.64 (m, 8H), 1.43 (s, 9H) ppm.

Step 4: Preparation of tert-butyl (3-(2-hydroxyethyl)bicyclo[1.1.1]pentan-1-yl)carbamate

To a solution of 2-(3-((tert-butoxycarbonyl)amino)bicyclo[1.1.1]pentan-1-yl)acetic acid (550 mg, 2.28 mmol, 1 eq) in THF (10 mL) was added BH₃ in THF (1 M, 9.12 mL, 4 eq) at −78° C., and the mixture was kept stirring at −78° C. for 10 hours under N₂ atmosphere. After the completion of the reaction, the reaction mixture was quenched with 5 mL of MeOH, and the volatiles were evaporated under vacuum. The resulting tert-butyl (3-(2-hydroxyethyl)bicyclo[1.1.1]pentan-1-yl)carbamate (380 mg, 73.34% yield) was obtained as a colorless oil, which was used into the next step without further purification. ¹H NMR (400 MHz, METHANOL-d₄) δ 3.55 (t, J=7.0 Hz, 2H), 1.87-1.84 (m, 6H), 1.75 (t, J=7.0 Hz, 2H), 1.43 (s, 9H) ppm.

Step 5: Preparation of tert-butyl (3-(2-((4-bromopyridin-2-yl)oxy)ethyl)bicyclo[1.1.1]pentan-1-yl)carbamate

To a solution of tert-butyl (3-(2-hydroxyethyl)bicyclo[1.1.1]pentan-1-yl)carbamate (356.47 mg, 1.57 mmol, 1.2 eq) and 4-bromo-2-fluoropyridine (230 mg, 1.31 mmol, 1 eq) in DMF (8 mL) was added tBuOK (219.98 mg, 1.96 mmol, 1.5 eq), and the mixture was stirred at 20° C. for 12 hours under N₂ atmosphere. After the completion of the reaction, the reaction mixture was quenched with water (30 mL) and extracted with ethyl acetate (30 mL×2). The combined organic layers were washed with brine (60 mL×2), dried over Na₂SO₄, filtered, and the filtrate was concentrated under reduced pressure. The resulting residue was purified by column chromatography to afford tert-butyl (3-(2-((4-bromopyridin-2-yl)oxy)ethyl)bicyclo[1.1.1]pentan-1-yl)carbamate (380 mg, 64.48% yield) as a white solid. LCMS: 383.2/385.2 [M+H]⁺.

Step 6: Preparation of tert-butyl (3-(2-((4-((1-(tert-butyl)-3-((1S,3R)-3-((tert-butyldimethylsilyl)oxy)cyclopentyl)-1H-pyrazol-5-yl)amino)pyridin-2-yl)oxy)ethyl)bicyclo[1.1.1]pentan-1-yl)carbamate

A mixture of tert-butyl (3-(2-((4-bromopyridin-2-yl)oxy)ethyl)bicyclo[1.1.1]pentan-1-yl)carbamate (408.74 mg, 1.07 mmol, 1.2 eq), 1-(tert-butyl)-3-((1S,3R)-3-((tert-butyldimethylsilyl)oxy)cyclopentyl)-1H-pyrazol-5-amine (300 mg, 0.889 mmol, 1 eq), Cs₂CO₃ (868.66 mg, 2.67 mmol, 3 eq), Xantphos (102.84 mg, 0.178 mmol, 0.2 eq) and Pd₂(dba)₃ (81.38 mg, 0.0888 mmol, 0.1 eq) in dioxane (5 mL) was heated and stirred at 90° C. for 2 hours under N₂ atmosphere. After the completion of the reaction, the reaction mixture was cooled to room temperature, filtered through a short pad of celite, rinsed with dioxane (10 mL) and the filtrate was concentrated under reduced pressure. The resulting residue was purified by column chromatography to afford tert-butyl (3-(2-((4-((1-(tert-butyl)-3-((1S,3R)-3-((tert-butyldimethylsilyl)oxy)cyclopentyl)-1H-pyrazol-5-yl)amino)pyridin-2-yl)oxy)ethyl)bicyclo[1.1.1]pentan-1-yl)carbamate (450 mg, 75.17% yield) as a white solid. LCMS: 640.6 [M+H]⁺.

Step 7: Preparation of tert-butyl (2-(2-(3-((tert-butoxycarbonyl)amino)bicyclo[1.1.1]pentan-1-yl)ethoxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (3-(2-((4-((1-(tert-butyl)-3-((1S,3R)-3-((tert-butyldimethylsilyl)oxy)cyclopentyl)-1H-pyrazol-5-yl)amino)pyridin-2-yl)oxy)ethyl)bicyclo[1.1.1]pentan-1-yl)carbamate (450 mg, 0.703 mmol, 1 eq) in THF (10 mL) was added Boc₂O (306.94 mg, 1.41 mmol, 323.09 μL, 2 eq), DMAP (85.91 mg, 0.703 mmol, 1 eq) and TEA (213.47 mg, 2.11 mmol, 293.62 μL, 3 eq). The mixture was stirred at 25° C. for 2 hours under N₂ atmosphere, and then the volatiles were removed under reduced pressure. The resulting residue was purified by column chromatography to afford tert-butyl (2-(2-(3-((tert-butoxycarbonyl)amino)bicyclo[1.1.1]pentan-1-yl)ethoxy)pyridin-4-yl)(1-(tert-butyl)-3-((1S,3R)-3-((tert-butyldimethylsilyl)oxy)cyclopentyl)-1H-pyrazol-5-yl)carbamate (430 mg, 80.98% yield) as yellow oil. LCMS: 740.4 [M+H]⁺.

Step 8: Preparation of tert-butyl (2-(2-(3-((tert-butoxycarbonyl)amino)bicyclo[1.1.1]pentan-1-yl)ethoxy)pyridin-4-yl)(1-(tert-butyl)-3-((1S,3R)-3-hydroxycyclopentyl)-1H-pyrazol-5-yl)carbamate

The solution of tert-butyl (2-(2-(3-((tert-butoxycarbonyl)amino)bicyclo[1.1.1]pentan-1-yl)ethoxy)pyridin-4-yl)(1-(tert-butyl)-3-((1S,3R)-3-((tert-butyldimethylsilyl)oxy)cyclopentyl)-1H-pyrazol-5-yl)carbamate (380 mg, 0.513 mmol, 1 eq) and TBAF (1 M, 2.05 mL, 4 eq) in THF (4 mL) was stirred at 25° C. for 2 hours under N₂ atmosphere, and then poured into water (5 mL). The aqueous solution was extracted with ethyl acetate (3×10 mL). The combined organic layer was washed with brine (10 mL), dried over anhydrous sodium sulfate, filtered and the filtrate was concentrated in vacuo. The resulting residue was purified by prep-TLC (SiO₂, petroleum ether: ethyl acetate=1:1, R_{f}=0.3) to afford tert-butyl (2-(2-(3-((tert-butoxycarbonyl)amino)bicyclo[1.1.1]pentan-1-yl)ethoxy)pyridin-4-yl)(1-(tert-butyl)-3-((1S,3R)-3-hydroxycyclopentyl)-1H-pyrazol-5-yl)carbamate (240 mg, 74.69% yield) as a colorless oil. LCMS: 626.5 [M+H]⁺.

Step 9: Preparation of tert-butyl (2-(2-(3-((tert-butoxycarbonyl)amino)bicyclo[1.1.1]pentan-1-yl)ethoxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a mixture of tert-butyl (2-(2-(3-((tert-butoxycarbonyl)amino)bicyclo[1.1.1]pentan-1-yl)ethoxy)pyridin-4-yl)(1-(tert-butyl)-3-((1S,3R)-3-hydroxycyclopentyl)-1H-pyrazol-5-yl)carbamate (120 mg, 0.192 mmol, 1 eq), Py (75.84 mg, 0.959 mmol, 77.39 μL, 5 eq) and DMAP (11.71 mg, 0.096 mmol, 0.5 eq) in THF (1 mL) was added a solution of 4-nitrophenyl carbonochloridate (115.95 mg, 0.575 mmol, 3 eq) in DCM (1 mL) dropwise at 0° C., and the mixture was stirred at 25° C. for 12 hours under N₂ atmosphere. After the completion of the reaction, the volatiles were removed under reduced pressure. The resulting residue was purified by prep-TLC (SiO₂, petroleum ether: ethyl acetate=2:1, R_{f}=0.35) to afford tert-butyl (2-(2-(3-((tert-butoxycarbonyl)amino)bicyclo[1.1.1]pentan-1-yl)ethoxy)pyridin-4-yl)(1-(tert-butyl)-3-((1S,3R)-3-(((4-nitrophenoxy)carbonyl)oxy)cyclopentyl)-1H-pyrazol-5-yl)carbamate (85 mg, 50.16% yield) as a white solid. LCMS: 791.4 [M+H]⁺.

Step 10: Preparation of (1R,3S)-3-(5-((2-(2-(3-aminobicyclo[1.1.1]pentan-1-yl)ethoxy)pyridin-4-yl)amino)-1-(tert-butyl)-1H-pyrazol-3-yl)cyclopentyl (4-nitrophenyl) carbonate

The mixture of tert-butyl (2-(2-(3-((tert-butoxycarbonyl)amino)bicyclo[1.1.1]pentan-1-yl)ethoxy)pyridin-4-yl)(1-(tert-butyl)-3-((1S,3R)-3-(((4-nitrophenoxy)carbonyl)oxy)cyclopentyl)-1H-pyrazol-5-yl)carbamate (85 mg, 0.107 mmol, 1 eq) and TFA (0.1 mL) in DCM (0.5 mL) was stirred at 25° C. for 12 hours under N₂ atmosphere. The volatiles were then removed under reduced pressure to afford (1R,3S)-3-(5-((2-(2-(3-aminobicyclo[1.1.1]pentan-1-yl)ethoxy)pyridin-4-yl)amino)-1-(tert-butyl)-1H-pyrazol-3-yl)cyclopentyl (4-nitrophenyl) carbonate (70 mg, crude) as a colorless oil, which was used in the next step without further purification. LCMS: 591.4 [M+H]⁺.

Step 11: Preparation of (1¹S,1³R,Z)-2¹-(tert-butyl)-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one

To a solution of (1R,3S)-3-(5-((2-(2-(3-aminobicyclo[1.1.1]pentan-1-yl)ethoxy)pyridin-4-yl)amino)-1-(tert-butyl)-1H-pyrazol-3-yl)cyclopentyl (4-nitrophenyl) carbonate (60 mg, 0.102 mmol, 1 eq) in DMF (20 mL) was added TEA (205.58 mg, 2.03 mmol, 282.77 μL, 20 eq), and the mixture was stirred at 100° C. for 0.5 hour under N₂ atmosphere. After the completion of the reaction, the mixture was cooled to room temperature and the volatiles were removed under reduced pressure. The remaining crude product was purified by prep-TLC (SiO₂, petroleum ether: ethyl acetate=1:1, R_{f}=0.57) to afford (1¹S,1³R,Z)-2¹-(tert-butyl)-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one (35 mg, 75.31% yield) as a yellow solid. LCMS: 452.3 [M+H]⁺.

Step 12: Preparation of (1¹S,1³R,Z)-2¹H-5,11-dioxa-3,9-diaza-4(4,2)-pyridina-2(3,5)-pyrazola-8(1,3)-bicyclo[1 0.1 0.1]pentana-1 (1,3)-cyclopentanacycloundecaphan-10-one

The solution of (1¹S,1³R,Z)-2¹-(tert-butyl)-2¹H-5,11-dioxa-3,9-diaza-4(4,2)-pyridina-2(3,5)-pyrazola-8(1,3)-bicyclo[1.1.1]pentana-1 (1,3)-cyclopentanacycloundecaphan-10-one (35 mg, 0.0775 mmol, 1 eq) in formic acid (1 mL) was stirred at 100° C. for 0.5 hour under N₂ atmosphere. The mixture was then cooled to room temperature and concentrated under reduced pressure. The crude product was purified by prep-HPLC (column: Phenomenex Luna C18 150×25 mm, 10 um; mobile phase: [water (FA)-ACN]; gradient: 10%-30% B over 10 min) to afford (1¹S,1³R,Z)-2¹H-5,11-dioxa-3,9-diaza-4(4,2)-pyridina-2(3,5)-pyrazola-8(1,3)-bicyclo[1 0.1 0.1]pentana-1(1,3)-cyclopentanacycloundecaphan-10-one (13.18 mg, 42.88% yield) as a white solid. LCMS: 396.3 [M+H]⁺; ¹H NMR (400 MHz, DMSO-d₆) δ 12.58-12.09 (m, 1H), 8.81 (s, 1H), 7.79 (d, J=5.6 Hz, 1H), 7.42 (s, 1H), 6.46 (d, J=5.2 Hz, 1H), 6.29 (s, 1H), 6.02 (s, 1H), 5.12 (d, J=4.0 Hz, 1H), 4.32-4.14 (m, 2H), 3.21-3.08 (m, 1H), 2.63-2.55 (m, 1H), 2.04-1.93 (m, 1H), 1.91-1.72 (m, 10H), 1.72-1.63 (m, 2H) ppm.

Example 8: Synthesis of (1'S,3′R,Z)-spiro[cyclopropane-1 5′-6,12-dioxa-3,10-diaza-4(4,2)-pyridina-2(3,5)-pyrazola-1(1,3)-cyclopentanacyclododecaphan]-11′-one (compound 26)

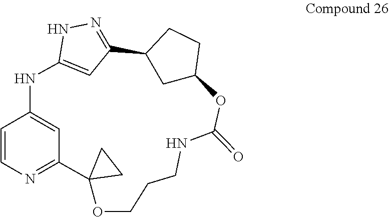 [SMILES: O=C1NCCCOC2(CC2)c2cc(ccn2)Nc2cc(n[nH]2)[C@H]2CC[C@H](C2)O1]

Step 1: Preparation of 4-bromopyridine-2-carboxylic acid

To a solution of methyl 4-bromopyridine-2-carboxylate (10 g, 46.29 mmol, 1 eq) in a co-solvent of THF (150 mL)-MeOH (50 mL)-H₂O (50 mL) was added LiOH·H₂O (7.77 g, 185.16 mmol, 4 eq), and the mixture was stirred at 25° C. for 16 hours. The volatiles were evaporated under reduced pressure and the residue was acidified to pH<3 with 4N HCl. The aqueous phase was extracted with DCM (100 mL×3). The combined organic layers were washed with brine (50 mL), dried over Na₂SO₄, filtered, and the filtrate was concentrated under reduced pressure to give 4-bromopyridine-2-carboxylic acid (5.8 g, 62.02% yield). LCMS: 202.1, 204.1 [M+H]⁺; ¹H NMR (400 MHz, DMSO-d₆) δ 8.60 (d, J=5.2 Hz, 1H), 8.19 (d, J=1.6 Hz, 1H), 7.93 (dd, J=1.6, 4.8 Hz, 1H) ppm.

Step 2: Preparation of 4-bromo-N-methoxy-N-methylpicolinamide

To a solution of 4-bromopyridine-2-carboxylic acid (5.80 g, 28.71 mmol, 1 eq), N-methoxymethanamine hydrochloride (4.20 g, 43.07 mmol, 1.5 eq) and HATU (16.38 g, 43.07 mmol, 1.5 eq) in DCM (100 mL) was added DIEA (11.13 g, 86.14 mmol, 15.00 mL, 3 eq), and the mixture was stirred at 25° C. for 2 hours. The mixture was then poured into water (50 mL). The organic phase was collected, and the aqueous phase was extracted with DCM (50 mL×4). The combined organic layers were washed with brine (30 mL), dried over anhydrous sodium sulfate, filtered, and the filtrate was concentrated in vacuo. The resulting residue was purified by column chromatography to afford 4-bromo-N-methoxy-N-methylpicolinamide (6.4 g, 61.96% yield) as a yellow oil. LCMS: 244.7, 246.7 [M+H]⁺.

Step 3: Preparation of 1-(4-bromo-2-pyridyl)ethanone

To a solution of 4-bromo-N-methoxy-N-methylpicolinamide (4.97 g, 20.30 mmol, 1 eq) in THF (100 mL) was added MeMgBr (3 M, 10.15 mL, 1.5 eq) dropwise at 0° C. under nitrogen atmosphere. After the completion of the addition, the reaction mixture was warmed to 25° C. and stirred for an additional 2 hours under N₂ atmosphere. The reaction mixture was then quenched with saturated aqueous NH₄Cl (100 mL) and the aqueous phase was extracted with DCM (100 mL×3). The combined organic layers were washed with brine (50 mL), dried over Na₂SO₄, filtered, and the filtrate was concentrated under reduced pressure. The resulting residue was purified by column chromatography to afford 1-(4-bromo-2-pyridyl)ethanone (4.1 g, 87.86% yield) as a yellow oil. LCMS: 199.8, 201.8 [M+H]⁺; ¹H NMR (400 MHz, CHLOROFORM-d) δ 8.56-8.43 (m, 1H), 8.31-7.99 (m, 1H), 7.61 (dd, J=1.9, 5.2 Hz, 1H), 2.68 (s, 3H) ppm.

Step 4: Preparation of 4-bromo-2-(1-((tert-butyldimethylsilyl)oxy)vinyl)pyridine

To a solution of 1-(4-bromo-2-pyridyl)ethanone (3.1 g, 15.50 mmol, 1 eq) and [tert-butyl(dimethyl)silyl] trifluoromethanesulfonate (4.96 g, 18.75 mmol, 4.31 mL, 1.21 eq) in DCM (60 mL) was added TEA (4.70 g, 46.49 mmol, 6.47 mL, 3 eq) at 0° C., and the mixture was warmed to 25° C. and stirred for 2 hours under N₂ atmosphere. After the completion of the reaction, the mixture was filtered, and the filtrate was concentrated. The resulting crude product was purified by column chromatography to afford 4-bromo-2-(1-((tert-butyldimethylsilyl)oxy)vinyl)pyridine (4.67 g, 90.66% yield) as a yellow oil. LCMS: 314.0, 316.0 [M+H]⁺; ¹H NMR (400 MHz, CHLOROFORM-d) δ 8.27 (d, J=5.1 Hz, 1H), 7.71 (d, J=1.8 Hz, 1H), 7.31-7.23 (m, 1H), 5.59 (d, J=1.1 Hz, 1H), 4.51 (s, 1H), 0.94 (s, 9H), 0.17 (s, 6H) ppm.

Step 5: Preparation of 4-bromo-2-(1-((tert-butyldimethylsilyl)oxy)cyclopropyl)pyridine

To a solution of ZnEt₂ (1 M, 44.80 mL, 3.2 eq) in DCM (64 mL) was added dropwise chloro(iodo)methane (15.83 g, 89.74 mmol, 6.51 mL, 6.41 eq) at 0° C., and the mixture was stirred at 0° C. for 20 minutes under N₂ atmosphere, followed by the dropwise addition of a solution of 4-bromo-2-(1-((tert-butyldimethylsilyl)oxy)vinyl)pyridine (4.4 g, 14.00 mmol, 1 eq) in DCM (32 mL) at 0° C. under N₂ atmosphere. After the completion of the addition, the reaction was kept stirring at 0° C. for 1 hour under N₂ atmosphere. The reaction was then quenched with saturated aqueous NH₄Cl(15 mL). The organic phase was isolated, and the aqueous phase was extracted with DCM (100 mL×3). The combined organic layers were washed with brine (50 mL), dried over Na₂SO₄, filtered, and the filtrate was concentrated under reduced pressure. The resulting residue was purified by column chromatography to afford 4-bromo-2-(1-((tert-butyldimethylsilyl)oxy)cyclopropyl)pyridine (2.5 g, 44.06% yield) as a white solid. LCMS: 327.8, 329.8 [M+H]⁺.

Step 6: Preparation of 1-(4-bromopyridin-2-yl)cyclopropan-1-ol

To a solution of 4-bromo-2-(1-((tert-butyldimethylsilyl)oxy)cyclopropyl)pyridine (2.5 g, 7.61 mmol, 1 eq) in MeOH (80 mL) was added NH₄F (8.46 g, 228.44 mmol, 30 eq), and the mixture was stirred at 60° C. for 1 hour. The reaction mixture was then cooled to room temperature and the volatiles were removed under reduced pressure. The resulting residue was purified by column chromatography to give 1-(4-bromopyridin-2-yl)cyclopropan-1-ol (1.58 g, 52.34% yield) as a yellow oil. LCMS: 213.8, 215.8 [M+H]⁺; 1H NMR (400 MHz, CHLOROFORM-d) δ 8.31 (d, J=5.3 Hz, 1H), 7.53 (d, J=1.6 Hz, 1H), 7.34-7.26 (m, 1H), 3.78-3.18 (m, 1H), 1.45-1.23 (m, 4H) ppm.

Step 7: Preparation of tert-butyl (3-(1-(4-bromopyridin-2-yl)cyclopropoxy)propyl)carbamate

To a solution of 1-(4-bromopyridin-2-yl)cyclopropan-1-ol (800 mg, 3.74 mmol, 1 eq) in DMF (9 mL) was added NaH (448.47 mg, 11.21 mmol, 60% purity, 3 eq) at 0° C., and the mixture was stirred for 0.5 hour at that temperature under N₂ atmosphere, followed by the addition of a solution of tert-butyl (3-bromopropyl)carbamate (4.45 g, 18.69 mmol, 5 eq) in DMF (9 mL). After the completion of the addition, the mixture was kept stirring at 0° C. for 1 hour under N₂ atmosphere. The mixture was then quenched with 0.5 mL of saturated aqueous NH₄Cl, and the volatiles were subsequently removed under reduced pressure. The remaining residue was purified by column chromatography to afford tert-butyl (3-(1-(4-bromopyridin-2-yl)cyclopropoxy)propyl)carbamate (474 mg, 27.67% yield) as a white solid. LCMS: 370.8, 372.8 [M+H]⁺.

Step 8: Preparation of tert-butyl (3-(1-(4-((1-(tert-butyl)-3-((1S,3R)-3-((tert-butyldimethylsilyl)oxy)cyclopentyl)-1H-pyrazol-5-yl)amino)pyridin-2-yl)cyclopropoxy)propyl)carbamate

The mixture of tert-butyl (3-(1-(4-bromopyridin-2-yl)cyclopropoxy)propyl)carbamate (360 mg, 0.97 mmol, 1.2 eq), 1-(tert-butyl)-3-((1S,3R)-3-((tert-butyldimethylsilyl)oxy)cyclopentyl)-1H-pyrazol-5-amine (272.77 mg, 0.808 mmol, 1 eq), Cs₂CO₃ (789.83 mg, 2.42 mmol, 3 eq), Pd₂(dba)₃ (73.99 mg, 0.081 mmol, 0.1 eq) and Xantphos (93.51 mg, 0.161 mmol, 0.2 eq) in dioxane (6 mL) was heated and stirred at 90° C. for 2 hours under N₂ atmosphere. After the completion of the reaction, the reaction mixture was cooled to room temperature, filtered through a short pad of celite, rinsed with dioxane (10 mL) and the filtrate was concentrated under reduced pressure. The resulting crude product was purified by column chromatography to afford tert-butyl (3-(1-(4-((1-(tert-butyl)-3-((1S,3R)-3-((tert-butyldimethylsilyl)oxy)cyclopentyl)-1H-pyrazol-5-yl)amino)pyridin-2-yl)cyclopropoxy)propyl)carbamate (484 mg, 90.20% yield) as a white solid. LCMS: 628.4 [M+H]⁺.

Step 9: Preparation of tert-butyl (2-(1-(3-((tert-butoxycarbonyl)amino)propoxy)cyclopropyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (3-(1-(4-((1-(tert-butyl)-3-((1S,3R)-3-((tert-butyldimethylsilyl)oxy)cyclopentyl)-1H-pyrazol-5-yl)amino)pyridin-2-yl)cyclopropoxy)propyl)carbamate (480 mg, 0.764 mmol, 1 eq) in DCM (10 mL) was added Boc₂O (333.67 mg, 1.53 mmol, 351.23 μL, 2 eq) and DMAP (93.39 mg, 0.764 mmol, 1 eq), and the mixture was stirred at 25° C. for 1 hour. The volatiles were then removed under reduced pressure. The resulting residue was purified by column chromatography to afford tert-butyl (2-(1-(3-((tert-butoxycarbonyl)amino)propoxy)cyclopropyl)pyridin-4-yl)(1-(tert-butyl)-3-((1S,3R)-3-((tert-butyldimethylsilyl)oxy)cyclopentyl)-1H-pyrazol-5-yl)carbamate (480 mg, 52.61% yield) as a yellow oil. LCMS: 728.5 [M+H]⁺.

Step 10: Preparation of tert-butyl (2-(1-(3-((tert-butoxycarbonyl)amino)propoxy)cyclopropyl)pyridin-4-yl)(1-(tert-butyl)-3-((1S,3R)-3-hydroxycyclopentyl)-1H-pyrazol-5-yl)carbamate

The solution of tert-butyl (2-(1-(3-((tert-butoxycarbonyl)amino)propoxy)cyclopropyl)pyridin-4-yl)(1-(tert-butyl)-3-((1S,3R)-3-((tert-butyldimethylsilyl)oxy)cyclopentyl)-1H-pyrazol-5-yl)carbamate (250 mg, 0.343 mmol, 1 eq) and TBAF (1 M, 0.687 mmol, 686.77 μL, 2 eq) in THF (3 mL) was stirred at 60° C. for 2 hours. After the completion of the reaction, the mixture was cooled to room temperature and the volatiles were evaporated under reduced pressure. The remaining residue was purified by prep-TLC (PE/EA=1/1) to afford tert-butyl (2-(1-(3-((tert-butoxycarbonyl)amino)propoxy)cyclopropyl)pyridin-4-yl)(1-(tert-butyl)-3-((1S,3R)-3-hydroxycyclopentyl)-1H-pyrazol-5-yl)carbamate (186 mg, 88.25% yield) as a yellow oil. LCMS: 614.5 [M+H]⁺.

Step 11: Preparation of tert-butyl (2-(1-(3-((tert-butoxycarbonyl)amino)propoxy)cyclopropyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(1-(3-((tert-butoxycarbonyl)amino)propoxy)cyclopropyl)pyridin-4-yl)(1-(tert-butyl)-3-((1S,3R)-3-hydroxycyclopentyl)-1H-pyrazol-5-yl)carbamate (186 mg, 0.303 mmol, 1 eq), Py (119.85 mg, 1.52 mmol, 122.30 μL, 5 eq) and DMAP (18.51 mg, 0.152 mmol, 0.5 eq) in THF (2 mL) was added a solution of 4-nitrophenyl carbonochloridate (183.24 mg, 0.909 mmol, 3 eq) in DCM (2 mL), and the mixture was stirred at 20° C. for 12 hours under N₂ atmosphere. After the completion of the reaction, the mixture was quenched with 0.5 mL of saturated aqueous NH₄Cl and the volatiles were subsequently removed under reduced pressure. The remaining residue was purified by column chromatography to afford tert-butyl (2-(1-(3-((tert-butoxycarbonyl)amino)propoxy)cyclopropyl)pyridin-4-yl)(1-(tert-butyl)-3-((1S,3R)-3-(((4-nitrophenoxy)carbonyl)oxy)cyclopentyl)-1H-pyrazol-5-yl)carbamate (200 mg, 59.31% yield) as a yellow solid. LCMS: 779.5 [M+H]⁺.

Step 12: Preparation of (1R,3S)-3-(5-((2-(1-(3-aminopropoxy)cyclopropyl)pyridin-4-yl)amino)-1-(tert-butyl)-1H-pyrazol-3-yl)cyclopentyl (4-nitrophenyl) carbonate

The solution of tert-butyl (2-(1-(3-((tert-butoxycarbonyl)amino)propoxy)cyclopropyl)pyridin-4-yl)(1-(tert-butyl)-3-((1S,3R)-3-(((4-nitrophenoxy)carbonyl)oxy)cyclopentyl)-1H-pyrazol-5-yl)carbamate (200 mg, 0.257 mmol, 1 eq) and TFA (3.07 g, 26.92 mmol, 2 mL, 104.86 eq) in DCM (8 mL) was stirred at 30° C. for 0.5 hour. The volatiles were then removed under reduced pressure to give (1R,3S)-3-(5-((2-(1-(3-aminopropoxy)cyclopropyl)pyridin-4-yl)amino)-1-(tert-butyl)-1H-pyrazol-3-yl)cyclopentyl (4-nitrophenyl) carbonate, which was used for the next step without further purification. LCMS: 579.2 [M+H]⁺.

Step 13: Preparation of (1'S,3′R,Z)-1′-(tert-butyl)spiro[cyclopropane-1,5′-6,12-dioxa-3,10-diaza-4(4,2)-pyridina-2(3,5)-pyrazola-1(1,3)-cyclopentanacyclododecaphan]-11′-one

To a solution of (1R,3S)-3-(5-((2-(1-(3-aminopropoxy)cyclopropyl)pyridin-4-yl)amino)-1-(tert-butyl)-1H-pyrazol-3-yl)cyclopentyl (4-nitrophenyl) carbonate (132 mg) in THF (50 mL) was added TEA (461.65 mg, 4.56 mmol, 635.01 μL, 20 eq), and the mixture was stirred at 25° C. for 1 hour under N₂ atmosphere. After the completion of the reaction, the volatiles were removed under reduced pressure. The resulting residue was purified by column chromatography to afford (1'S,3′R,Z)-1′-(tert-butyl)spiro[cyclopropane-1,5′-6,12-dioxa-3,10-diaza-4(4,2)-pyridina-2(3,5)-pyrazola-1(1,3)-cyclopentanacyclododecaphan]-11′-one (33 mg, 30.28% yield) as a yellow oil. LCMS: 440.3 [M+H]⁺.

Step 14: Preparation of (1'S,3′R,Z)-spiro[cyclopropane-1,5′-6,12-dioxa-3,10-diaza-4(4,2)-pyridina-2(3,5)-pyrazola-1(1,3)-cyclopentanacyclododecaphan]-11′-one

The solution of (1'S,3′R,Z)-1′-(tert-butyl)spiro[cyclopropane-1,5′-6,12-dioxa-3,10-diaza-4(4,2)-pyridina-2(3,5)-pyrazola-1(1,3)-cyclopentanacyclododecaphan]-11′-one (30 mg, 0.0683 mmol, 1 eq) in formic acid (1 mL) was stirred at 100° C. for 0.5 hour under N₂ atmosphere. The reaction mixture was then cooled to room temperature and concentrated under reduced pressure. The remaining residue was purified by prep-HPLC (column: Phenomenex Luna C18 150×25 mm, 10 um; mobile phase: [water (FA)-ACN]; gradient: 6%-36% B over 10 min) to afford (1'S,3′R,Z)-spiro[cyclopropane-1,5′-6,12-dioxa-3,10-diaza-4(4,2)-pyridina-2(3,5)-pyrazola-1(1,3)-cyclopentanacyclododecaphan]-11′-one (15.35 mg, 57.25% yield) as a white solid. LCMS: 384.0 [M+H]⁺; ¹H NMR (400 MHz, DMSO-d₆) δ 12.5 (br s, 1H), 8.89 (s, 1H), 8.15 (s, 1H), 7.96 (d, J=5.6 Hz, 1H), 7.16-7.10 (m, 1H), 6.58-6.57 (m, 1H), 6.57 (dd, J=2.3, 3.2 Hz, 1H), 5.96-5.90 (s, 1H), 5.05-5.04 (m, 1H), 3.23-3.20 (m, 2H), 3.18-2.66 (m, 2H), 2.10-1.92 (m, 2H), 1.90-1.71 (m, 6H), 1.65-1.50 (m, 1H), 1.30-1.20 (m, 1H), 1.07 (br s, 3H) ppm.

Example 9: Synthesis of (1'S,3′R,Z)-spiro[cyclopropane-1 9′-5,12-dioxa-3,10-diaza-4(4,2)-pyridina-2(3,5)-pyrazola-1(1,3)-cyclopentanacyclododecaphan]-11′-one (Compound 33)

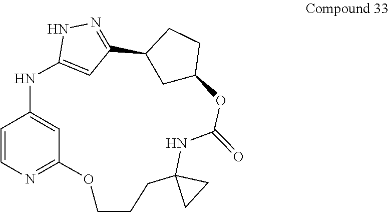 [SMILES: O=C1NC2(CCCOc3cc(ccn3)Nc3cc(n[nH]3)[C@H]3CC[C@H](C3)O1)CC2]

Step 1: Preparation of ethyl (E)-3-[1-(tert-butoxycarbonylamino)cyclopropyl]prop-2-enoate

The mixture of tert-butyl N-(1-formylcyclopropyl)carbamate (4.5 g, 24.30 mmol, 1 eq) and ethyl 2-(triphenyl-phosphanylidene)acetate (9.31 g, 26.73 mmol, 1.1 eq) in DCM (40 mL) was stirred at 20° C. for 12 hours under N₂ atmosphere. The volatiles were then evaporated and the remaining residue was purified by column chromatography to afford (E)-3-[1-(tert-butoxycarbonylamino)cyclopropyl]prop-2-enoate (5.5 g, 88.67% yield) as a white solid. 1H NMR (400 MHz, DMSO-d₆) δ 7.49 (br s, 1H), 6.50 (d, J=15.4 Hz, 1H), 5.63 (br d, J=15.2 Hz, 1H), 4.15-4.03 (m, 2H), 1.38 (s, 9H), 1.20 (t, J=7.0 Hz, 3H), 1.14-1.04 (m, 4H) ppm.

Step 2: Preparation of ethyl 3-[1-(tert-butoxycarbonylamino)cyclopropyl]propanoate

The mixture of Pd/C (5%, 250.10 mg) and ethyl (E)-3-[1-(tert-butoxycarbonylamino)cyclopropyl]prop-2-enoate (5 g, 19.58 mmol, 1 eq) in ethyl acetate (100 mL) was degassed and backfilled with H₂ for 3 times, and stirred under H₂ (15 psi) atmosphere at 20° C. for 3 hours. The solid was filtered off, rinsed with ethyl acetate (20 mL) and the filtrate was concentrated under reduced pressure. The desired ethyl 3-[1-(tert-butoxycarbonylamino)cyclopropyl]propanoate (600 mg, 75.72% yield) was obtained as a white solid. ¹H NMR (400 MHz, CHLOROFORM-d) δ 4.88 (br s, 1H), 4.15 (q, J=7.2 Hz, 2H), 1.96-1.78 (m, 2H), 1.62 (br s, 2H), 1.57-1.40 (m, 9H), 1.31-1.25 (m, 3H), 0.78 (br s, 2H), 0.70-0.60 (m, 2H) ppm.

Step 3: Preparation of tert-butyl (1-(3-hydroxypropyl)cyclopropyl)carbamate

To a solution of ethyl 3-[1-(tert-butoxycarbonylamino)cyclopropyl]propanoate (200 mg, 0.777 mmol, 1 eq) in THF (2 mL) was added LiBH₄ (2 M, 2.13 mL, 5.48 eq), and the mixture was stirred at 25° C. for 18 hours under N₂ atmosphere. The reaction mixture was quenched with 50% aqueous acetic acid (2 mL), and then diluted with H₂O (10 mL). The aqueous phase was extracted with EtOAc (20 mL×3). The combined organic layers were washed with saturated aqueous NaHCO₃(10 mL), and brine (10 mL), dried over Na₂SO₄, filtered and the filtrate was concentrated under reduced pressure. The resulting residue was purified by column chromatography to afford tert-butyl (1-(3-hydroxypropyl)cyclopropyl)carbamate (96 mg, 57.37% yield) as a brown oil. ¹H NMR (400 MHz, CHLOROFORM-d) δ 3.72-3.65 (m, 2H), 1.71-1.66 (m, 2H), 1.63-1.58 (m, 2H), 1.44 (d, J=3.2 Hz, 9H), 0.79-0.72 (m, 2H), 0.67-0.58 (m, 2H) ppm.

Step 4: Preparation of tert-butyl (1-(3-((4-bromopyridin-2-yl)oxy)propyl)cyclopropyl)carbamate

The mixture of tert-butyl (1-(3-hydroxypropyl)cyclopropyl)carbamate (3.2 g, 14.86 mmol, 1.5 eq), 4-bromo-2-fluoro-pyridine (1.74 g, 9.91 mmol, 1 eq) and t-BuOK (1.67 g, 14.86 mmol, 1.5 eq) in DMF (30 mL) was stirred at 0° C. for 16 hours under N₂ atmosphere. The reaction was then quenched with H₂O (50 mL). The aqueous phase was extracted with EtOAc (50 mL×3). The combined organic phase was washed with brine (20 mL), dried over Na₂SO₄, filtered and the filtrate was concentrated under reduced pressure. The resulting residue was purified by flash silica gel chromatography to afford tert-butyl (1-(3-((4-bromopyridin-2-yl)oxy)propyl)cyclopropyl)carbamate (880 mg, 2.37 mmol, 23.92% yield) as a white solid. LCMS: 370.8/372.8 [M+H]⁺.

Step 5: Preparation of tert-butyl (1-(3-((4-((1-(tert-butyl)-3-((1S,3R)-3-((tert-butyldimethylsilyl)oxy)cyclopentyl)-1H-pyrazol-5-yl)amino)pyridin-2-yl)oxy)propyl)cyclopropyl)carbamate

The mixture of tert-butyl (1-(3-((4-bromopyridin-2-yl)oxy)propyl)cyclopropyl)carbamate (420 mg, 1.13 mmol, 1.2 eq), 1-(tert-butyl)-3-((1S,3R)-3-((tert-butyldimethylsilyl)oxy)cyclopentyl)-1H-pyrazol-5-amine (318.24 mg, 0.943 mmol, 1 eq), Cs₂CO₃ (921.46 mg, 2.83 mmol, 3 eq), Pd₂(dba)₃ (86.33 mg, 0.0943 mmol, 0.1 eq) and Xantphos (109.09 mg, 0.189 mmol, 0.2 eq) in dioxane (4 mL) was heated and stirred at 90° C. for 2 hours under N₂ atmosphere. The reaction was then cooled to room temperature, and the volatiles were evaporated under reduced pressure. The resulting residue was diluted with H₂O (40 mL), and the aqueous phase was extracted with EtOAc (50 mL×3). The combined organic phase was washed with brine (20 mL), dried over Na₂SO₄, filtered and the filtrate was concentrated under reduced pressure. The remaining residue was purified by column chromatography to afford tert-butyl (1-(3-((4-((1-(tert-butyl)-3-((1S,3R)-3-((tert-butyldimethylsilyl)oxy)cyclopentyl)-1H-pyrazol-5-yl)amino)pyridin-2-yl)oxy)propyl)cyclopropyl)carbamate (530 mg, 89.53% yield) as a yellow oil. LCMS: 628.3 [M+H]⁺.

Step 6: Preparation of tert-butyl (2-(3-(1-((tert-butoxycarbonyl)amino)cyclopropyl)propoxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (1-(3-((4-((1-(tert-butyl)-3-((1S,3R)-3-((tert-butyldimethylsilyl)oxy)cyclopentyl)-1H-pyrazol-5-yl)amino)pyridin-2-yl)oxy)propyl)cyclopropyl)carbamate (530 mg, 0.844 mmol, 1 eq), Boc₂O (368.42 mg, 1.69 mmol, 387.81 μL, 2 eq) and DMAP (103.11 mg, 0.844 mmol, 1 eq) in THF (6 mL) was added TEA (256.22 mg, 2.53 mmol, 352.44 μL, 3 eq), and the mixture was stirred at 25° C. for 2 hours under N₂ atmosphere. After the completion of the reaction, the volatiles were removed under reduced pressure. The resulting residue was purified by column chromatography to afford tert-butyl (2-(3-(1-((tert-butoxycarbonyl)amino)cyclopropyl)propoxy)pyridin-4-yl)(1-(tert-butyl)-3-((1S,3R)-3-((tert-butyldimethylsilyl)oxy)cyclopentyl)-1H-pyrazol-5-yl)carbamate (450 mg, 73.23% yield) as a yellow oil. LCMS: 728.7 [M+H]⁺.

Step 7: Preparation of tert-butyl (2-(3-(1-((tert-butoxycarbonyl)amino)cyclopropyl)propoxy)pyridin-4-yl)(1-(tert-butyl)-3-((1S,3R)-3-hydroxycyclopentyl)-1H-pyrazol-5-yl)carbamate

The mixture of tert-butyl (2-(3-(1-((tert-butoxycarbonyl)amino)cyclopropyl)propoxy)pyridin-4-yl)(1-(tert-butyl)-3-((1S,3R)-3-((tert-butyldimethylsilyl)oxy)cyclopentyl)-1H-pyrazol-5-yl)carbamate (448 mg, 0.615 mmol, 1 eq) and TBAF (1 M, 2.46 mL, 4 eq) in THF (4 mL) was stirred at 25° C. for 16 hours under N₂ atmosphere. After the completion of the reaction, the volatiles were removed under reduced pressure. The resulting residue was purified by column chromatography to afford tert-butyl (2-(3-(1-((tert-butoxycarbonyl)amino)cyclopropyl)propoxy)pyridin-4-yl)(1-(tert-butyl)-3-((1S,3R)-3-hydroxycyclopentyl)-1H-pyrazol-5-yl)carbamate (330 mg, 87.37% yield) as a white solid. LCMS: 614.4 [M+H]⁺; ¹H NMR (400 MHz, DMSO-d₆) δ 8.08 (d, J=5.8 Hz, 1H), 7.14 (br s, 1H), 6.81 (br d, J=5.0 Hz, 1H), 6.64 (s, 1H), 6.18 (s, 1H), 4.64-4.59 (m, 1H), 4.31-4.24 (m, 2H), 4.23-4.16 (m, 1H), 3.02 (br t, J=8.4 Hz, 1H), 1.84-1.74 (m, 4H), 1.69-1.51 (m, 6H), 1.43 (d, J=4.2 Hz, 18H), 1.39 (s, 9H), 0.68-0.46 (m, 4H) ppm.

Step 8: Preparation of tert-butyl (2-(3-(1-((tert-butoxycarbonyl)amino)cyclopropyl)propoxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(3-(1-((tert-butoxycarbonyl)amino)cyclopropyl)propoxy)pyridin-4-yl)(1-(tert-butyl)-3-((1S,3R)-3-hydroxycyclopentyl)-1H-pyrazol-5-yl)carbamate (300 mg, 0.489 mmol, 1 eq), Py (193.31 mg, 2.44 mmol, 197.25 μL, 5 eq) and DMAP (29.86 mg, 0.244 mmol, 0.5 eq) in THF (2 mL) was added a solution of (4-nitrophenyl)chloroformate (295.55 mg, 1.47 mmol, 3 eq) in DCM (2 mL) at 0° C., and the mixture was stirred at 0° C. for 12 hours under nitrogen atmosphere. The mixture was then quenched with saturated aqueous NH₄Cl (0.5 mL) and the volatiles were evaporated under reduced pressure. The resulting crude was purified by prep-TLC (SiO₂, PE:EA=2:1) to afford tert-butyl (2-(3-(1-((tert-butoxycarbonyl)amino)cyclopropyl)propoxy)pyridin-4-yl)(1-(tert-butyl)-3-((1S,3R)-3-(((4-nitrophenoxy)carbonyl)oxy)cyclopentyl)-1H-pyrazol-5-yl)carbamate (370 mg, 97.19% yield) as a colorless oil. LCMS: 779.3 [M+H]⁺.

Step 9: Preparation of (1R,3S)-3-(5-((2-(3-(1-aminocyclopropyl)propoxy)pyridin-4-yl)amino)-1-(tert-butyl)-1H-pyrazol-3-yl)cyclopentyl (4-nitrophenyl) carbonate

The mixture of tert-butyl (2-(3-(1-((tert-butoxycarbonyl)amino)cyclopropyl)propoxy)pyridin-4-yl)(1-(tert-butyl)-3-((1S,3R)-3-(((4-nitrophenoxy)carbonyl)oxy)cyclopentyl)-1H-pyrazol-5-yl)carbamate (170 mg, 0.218 mmol, 1 eq) and TFA (0.2 mL) in DCM (2 mL) was stirred at 25° C. for 12 hours under nitrogen atmosphere. The reaction mixture was then concentrated under reduced pressure to afford (1R,3S)-3-(5-((2-(3-(1-aminocyclopropyl)propoxy)pyridin-4-yl)amino)-1-(tert-butyl)-1H-pyrazol-3-yl)cyclopentyl (4-nitrophenyl) carbonate (180 mg, crude) as a yellow oil. LCMS: 579.3 [M+H]⁺.

Step 10: Preparation of (1'S,3′R,Z)-1′-(tert-butyl)spiro[cyclopropane-1,9′-5,12-dioxa-3,10-diaza-4(4,2)-pyridina-2(3,5)-pyrazola-1(1,3)-cyclopentanacyclododecaphan]-11′-one

To a solution of (1R,3S)-3-(5-((2-(3-(1-aminocyclopropyl)propoxy)pyridin-4-yl)amino)-1-(tert-butyl)-1H-pyrazol-3-yl)cyclopentyl (4-nitrophenyl) carbonate (180 mg) in DMSO (69.13 mL) was added TEA (279.79 mg, 2.77 mmol, 384.85 μL, 20 eq), and the mixture was stirred at 100° C. for 0.5 hour under nitrogen atmosphere. The mixture was then cooled to room temperature, diluted with H₂O (50 mL) and the aqueous phase was extracted with EtOAc (30 mL×3). The combined organic phase was washed with brine (20 mL), dried over Na₂SO₄, filtered and the filtrate was concentrated under reduced pressure. The resulting residue was purified by prep-HPLC (NH₃. H₂O condition) to afford (1'S,3′R,Z)-1′-(tert-butyl)spiro[cyclopropane-1,9′-5,12-dioxa-3,10-diaza-4(4,2)-pyridina-2(3,5)-pyrazola-1(1,3)-cyclopentanacyclododecaphan]-11′-one (30 mg, 49.37% yield) as a white solid. LCMS: 440.3 [M+H]⁺.

Step 11: Preparation of (1'S,3′R,Z)-spiro[cyclopropane-1,9′-5,12-dioxa-3,10-diaza-4(4,2)-pyridina-2(3,5)-pyrazola-1 (1,3)-cyclopentanacyclododecaphan]-11′-one

The solution of (1'S,3′R,Z)-1′-(tert-butyl)spiro[cyclopropane-1,9′-5,12-dioxa-3,10-diaza-4(4,2)-pyridina-2(3,5)-pyrazola-1(1,3)-cyclopentanacyclododecaphan]-11′-one (30 mg, 0.0683 mmol, 1 eq) in formic acid (0.5 mL) was stirred at 100° C. for 0.5 hour under nitrogen atmosphere. The reaction mixture was then cooled to room temperature and the volatiles were removed under reduced pressure. The resulting residue was purified by prep-HPLC (column: YMC-Actus Triart C18 150×30 mm, 7 um; mobile phase: [water(FA)-ACN]; gradient:13%-43% B over 10 min) to afford (1'S,3′R,Z)-spiro[cyclopropane-1 9′-5,12-dioxa-3,10-diaza-4(4,2)-pyridina-2(3,5)-pyrazola-1(1,3)-cyclopentanacyclododecaphan]-11′-one (16 mg, 60.53% yield) as a white solid. LCMS: 384.0 [M+H]⁺; ¹H NMR (400 MHz, DMSO-d₆) δ 12.23-12.01 (m, 1H), 9.02-8.93 (m, 1H), 7.79-7.70 (m, 1H), 7.54-7.45 (m, 1H), 6.47-6.32 (m, 2H), 6.07 (s, 1H), 5.08-4.94 (m, 1H), 4.24-4.12 (m, 1H), 3.99-3.89 (m, 1H), 3.24-3.14 (m, 1H), 2.10-1.97 (m, 2H), 1.97-1.89 (m, 1H), 1.89-1.67 (m, 6H), 1.23-1.08 (m, 1H), 0.64-0.48 (m, 4H) ppm.

Example 10: Synthesis of (1¹S,1³R,Z)-2¹H-5,12-dioxa-3,10-diaza-4(3,5)-pyridina-2(3,5)-pyrazola-1(1,3)-cyclopentanacyclododecaphan-11-one (compound 104)

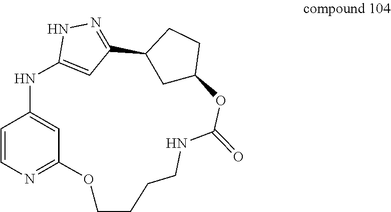 [SMILES: O=C1NCCCCOc2cc(ccn2)Nc2cc(n[nH]2)[C@H]2CC[C@H](C2)O1]

Step 1: Preparation of tert-butyl (4-((5-bromopyridin-3-yl)oxy)butyl)carbamate

A mixture of 5-bromopyridin-3-ol (500 mg, 2.87 mmol, 1 eq), tert-butyl N-(4-hydroxybutyl)carbamate (543.84 mg, 2.87 mmol, 1 eq) and 2-(tributyl-phosphanylidene)acetonitrile (1.04 g, 4.31 mmol, 1.5 eq) in toluene (3 mL) was degassed and purged with N₂ for 3 times, and then stirred at 80° C. for 2 hr under N₂ atmosphere. The reaction mixture was cooled to room temperature, filtered through a pad of Celite and rinsed with EtOAc (60 mL). The filtrate was concentrated under reduced pressure to give a crude product, which was purified by column chromatography (SiO₂, Petroleum ether/Ethyl acetate=100/1 to 20/1) to afford tert-butyl (4-((5-bromopyridin-3-yl)oxy)butyl)carbamate (724 mg, 2.10 mmol, 72.9% yield) as a red solid. LCMS: 345.1/347.1 [M+H]⁺.

Step 2: Preparation of tert-butyl (4-((5-((1-(tert-butyl)-3-((1S,3R)-3-((tert-butyldimethylsilyl)oxy)cyclopentyl)-1H-pyrazol-5-yl)amino)pyridin-3-yl)oxy)butyl)carbamate

A mixture of tert-butyl (4-((5-bromopyridin-3-yl)oxy)butyl)carbamate (245.44 mg, 0.711 mmol, 1.2 eq), 1-(tert-butyl)-3-((1S,3R)-3-((tert-butyldimethylsilyl)oxy)cyclopentyl)-1H-pyrazol-5-amine (200 mg, 0.592 mmol, 1 eq), Cs₂CO₃ (579.11 mg, 1.78 mmol, 3 eq), Pd₂(dba)₃ (54.25 mg, 0.0593 mmol, 0.1 eq) and XPhos (56.49 mg, 0.118 mmol, 0.2 eq) in dioxane (1 mL) was degassed and purged with N₂ for 3 times, and then stirred at 90° C. for 2 hr under N₂ atmosphere. The reaction mixture was cooled to room temperature, filtered and the filtrate was concentrated under reduced pressure. The resulting residue was purified by column chromatography to afford tert-butyl (4-((5-((1-(tert-butyl)-3-((1S,3R)-3-((tert-butyldimethylsilyl)oxy)cyclopentyl)-1H-pyrazol-5-yl)amino)pyridin-3-yl)oxy)butyl)carbamate (337 mg, 87.4% yield) as a yellow oil. LCMS: 602.5 [M+H]⁺.

Step 3: Preparation of tert-butyl (5-(4-((tert-butoxycarbonyl)amino)butoxy)pyridin-3-yl)(1-(tert-butyl)-3-((1S,3R)-3-((tert-butyldimethylsilyl)oxy)cyclopentyl)-1H-pyrazol-5-yl)carbamate

To a solution of tert-butyl (4-((5-((1-(tert-butyl)-3-((1S,3R)-3-((tert-butyldimethylsilyl)oxy)cyclopentyl)-1H-pyrazol-5-yl)amino)pyridin-3-yl)oxy)butyl)carbamate (300 mg, 0.498 mmol, 1 eq), DMAP (60.89 mg, 0.498 mmol, 1 eq) and Boc₂O (217.56 mg, 0.997 mmol, 229.01 μL, 2 eq) in THF (5 mL) was added TEA (151.31 mg, 1.50 mmol, 208.12 μL, 3 eq). The mixture was kept stirring at 25° C. for 2 hr, and then quenched with water (10 mL). The mixture was extracted with EtOAc (20 mL×3), and the combined organic layers were washed with brine (10 mL), dried over anhydrous sodium sulfate, filtered and the filtrate was concentrated under reduced pressure. The resulting residue was purified by prep-TLC (SiO₂, Petroleum ether/Ethyl acetate=2/1) to afford tert-butyl (5-(4-((tert-butoxycarbonyl)amino)butoxy)pyridin-3-yl)(1-(tert-butyl)-3-((1S,3R)-3-((tert-butyldimethylsilyl)oxy)cyclopentyl)-1H-pyrazol-5-yl)carbamate (283 mg, 73.12% yield) as a red oil. LCMS: 702.5 [M+H]⁺.

Step 4: Preparation of tert-butyl (5-(4-((tert-butoxycarbonyl)amino)butoxy)pyridin-3-yl)(1-(tert-butyl)-3-((1S,3R)-3-hydroxycyclopentyl)-1H-pyrazol-5-yl)carbamate

The mixture of tert-butyl (5-(4-((tert-butoxycarbonyl)amino)butoxy)pyridin-3-yl)(1-(tert-butyl)-3-((1S,3R)-3-((tert-butyldimethylsilyl)oxy)cyclopentyl)-1H-pyrazol-5-yl)carbamate (250 mg, 0.356 mmol, 1 eq) and TBAF (1 M, 1.42 mL, 4 eq) in THF (1 mL) was stirred at 25° C. for 16 hours. The mixture was then poured into water (30 mL) and extracted with ethyl acetate (30 mL×3). The combined organic layers were washed with brine (30 mL), dried over anhydrous sodium sulfate, filtered and the filtrate was concentrated in vacuo. The resulting crude product was purified by prep-TLC (SiO₂, DCM:MeOH=15:1) to afford tert-butyl (5-(4-((tert-butoxycarbonyl)amino)butoxy)pyridin-3-yl)(1-(tert-butyl)-3-((1S,3R)-3-hydroxycyclopentyl)-1H-pyrazol-5-yl)carbamate (150 mg, 71.1% yield) as a red oil. LCMS: 588.4 [M+H]⁺.

Step 5: tert-butyl (5-(4-((tert-butoxycarbonyl)amino)butoxy)pyridin-3-yl)(1-(tert-butyl)-3-((1S,3R)-3-(((4-nitrophenoxy)carbonyl)oxy)cyclopentyl)-1H-pyrazol-5-yl)carbamate

To a mixture of tert-butyl (5-(4-((tert-butoxycarbonyl)amino)butoxy)pyridin-3-yl)(1-(tert-butyl)-3-((1S,3R)-3-hydroxycyclopentyl)-1H-pyrazol-5-yl)carbamate (120 mg, 0.204 mmol, 1 eq), Py (80.75 mg, 1.02 mmol, 82.40 μL, 5 eq) and DMAP (12.47 mg, 0.102 mmol, 0.5 eq) in THF (1 mL) was added dropwise a solution of(4-nitrophenyl) carbonochloridate (123.46 mg, 0.613 mmol, 3 eq) in DCM (1 mL) at 0° C. After the completion of addition, the mixture was kept stirring at 25° C. for 12 hours. The volatiles were evaporated under reduced pressure to give a residue, which was purified by prep-TLC (SiO₂, Petroleum ether/Ethyl acetate=2/1) to afford tert-butyl (5-(4-((tert-butoxycarbonyl)amino)butoxy)pyridin-3-yl)(1-(tert-butyl)-3-((1S,3R)-3-(((4-nitrophenoxy)carbonyl)oxy)cyclopentyl)-1H-pyrazol-5-yl)carbamate (134 mg, 71.0% yield) as a white solid. LCMS: 753.4 [M+H]⁺.

Step 6: Preparation of (1R,3S)-3-(5-((5-(4-aminobutoxy)pyridin-3-yl)amino)-1-(tert-butyl)-1H-pyrazol-3-yl)cyclopentyl (4-nitrophenyl) carbonate

The mixture of tert-butyl (5-(4-((tert-butoxycarbonyl)amino)butoxy)pyridin-3-yl)(1-(tert-butyl)-3-((1S,3R)-3-(((4-nitrophenoxy)carbonyl)oxy)cyclopentyl)-1H-pyrazol-5-yl)carbamate (110 mg, 0.146 mmol, 1 eq) and TFA (0.1 mL) in DCM (0.5 mL) was stirred at 25° C. for 12 hours. The volatiles were evaporated under reduced pressure to give (1R,3S)-3-(5-((5-(4-aminobutoxy)pyridin-3-yl)amino)-1-(tert-butyl)-1H-pyrazol-3-yl)cyclopentyl (4-nitrophenyl) carbonate (70 mg, crude, TFA salt) as a yellow oil. LCMS: 553.3 [M+H]⁺.

Step 7: Preparation of (1¹S,1³R,Z)-2¹-(tert-butyl)-2¹H-5,12-dioxa-3,10-diaza-4(3,5)-pyridina-2(3,5)-pyrazola-1(1,3)-cyclopentanacyclododecaphan-11-one

To a solution of (1R,3S)-3-(5-((5-(4-aminobutoxy)pyridin-3-yl)amino)-1-(tert-butyl)-1H-pyrazol-3-yl)cyclopentyl (4-nitrophenyl) carbonate (70 mg, crude) in THF (54 mL) was added DIEA (70.16 mg, 0.543 mmol, 94.56 μL, 5 eq), and the mixture was stirred at 25° C. for 12 hours. The mixture was concentrated under reduced pressure and the remaining residue was purified prep-TLC to afford (1¹S,1³R,Z)-2¹-(tert-butyl)-2¹H-5,12-dioxa-3,10-diaza-4(3,5)-pyridina-2(3,5)-pyrazola-1(1,3)-cyclopentanacyclododecaphan-11-one (31 mg, 49.3% yield) as a yellow solid. LCMS: 414.3 [M+H]⁺.

Step 8: Preparation of (1¹S,1³R,Z)-2¹H-5,12-dioxa-3,10-diaza-4(3,5)-pyridina-2(3,5)-pyrazola-1 (1,3)-cyclopentanacyclododecaphan-11-one

A solution of (1¹S,1³R,Z)-2¹-(tert-butyl)-2¹H-5,12-dioxa-3,10-diaza-4(3,5)-pyridina-2(3,5)-pyrazola-1(1,3)-cyclopentanacyclododecaphan-11-one (26 mg, 0.0629 mmol, 1 eq) in formic acid (0.3 mL) was stirred at 100° C. for 0.5 hour under N₂ atmosphere. After the completion of the reaction, the reaction mixture was cooled to room temperature and concentrated under reduced pressure. The remaining residue was purified by prep-HPLC (column: Phenomenex luna C18 150*25 mm*10 um; mobile phase: [water (FA)-ACN]; gradient: 3%-33% B over 10 min) to give (1¹S,1³R,Z)-2¹H-5,12-dioxa-3,10-diaza-4(3,5)-pyridina-2(3,5)-pyrazola-1(1,3)-cyclopentanacyclododecaphan-11-one (10.69 mg, 45.8% yield) as a white solid. LCMS: 358.2 [M+H]⁺. ¹H NMR (400 MHz, DMSO-d₆) δ 11.97 (br s, 1H), 8.61-8.49 (m, 1H), 7.78 (d, J=2.0 Hz, 1H), 7.55 (d, J=1.9 Hz, 1H), 7.24-7.15 (m, 1H), 7.03-6.93 (m, 1H), 5.94 (s, 1H), 5.13-4.96 (m, 1H), 4.19-3.87 (m, 2H), 3.18-3.03 (m, 2H), 2.84-2.66 (m, 1H), 2.40-2.31 (m, 1H), 2.10-1.96 (m, 1H), 1.88-1.71 (m, 6H), 1.58-1.45 (m, 2H) ppm.

Example 11: Synthesis of (1¹S,1³R,Z)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one (compound 21)

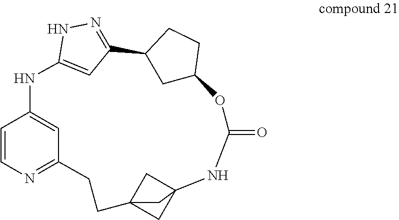 [SMILES: O=C1NC23CC(CCc4cc(ccn4)Nc4cc(n[nH]4)[C@H]4CC[C@H](C4)O1)(C2)C3]

Step 1: Preparation of tert-butyl (3-formylbicyclo[1.1.1]pentan-1-yl)carbamate

To a solution of (COCl)₂ (773.68 mg, 6.10 mmol, 533.57 μL, 1.3 eq) in DCM (10 mL) was added dropwise a solution of dimethyl sulfoxide (952.52 mg, 12.19 mmol, 952.52 μL, 2.6 eq) in DCM (10 mL) at −78° C. under N₂ atmosphere. After the completion of the addition, the mixture was stirred for 30 minutes at this temperature, and then a solution of tert-butyl (3-(hydroxymethyl)bicyclo[1.1.1]pentan-1-yl)carbamate (1 g, 4.69 mmol, 1 eq) in DCM (10 mL) was added dropwise at −78° C. under N₂ atmosphere. The reaction was kept at this temperature for an additional 30 minutes, and a solution of TEA (2.85 g, 28.13 mmol, 3.92 mL, 6 eq) in DCM (10 mL) was then dropwise added at −78° C. under N₂ atmosphere. After the completion of the addition, the resulting mixture was first stirred at −78° C. for 30 minutes, and then warmed to 20° C. and stirred for an additional 1 hour. After the starting material tert-butyl (3-(hydroxymethyl)bicyclo[1.1.1]pentan-1-yl)carbamate was completely consumed, the reaction mixture was concentrated under reduced pressure. The resulting residue was diluted with 100 mL of water and extracted with DCM (100 mL×2). The combined organic layers were washed with brine (50 mL), dried over Na₂SO₄, filtered and the filtrate was concentrated under reduced pressure to give tert-butyl (3-formylbicyclo[1.1.1]pentan-1-yl)carbamate (1 g, crude) as a yellow solid. ¹H NMR (400 MHz, DMSO-d₆) δ 9.59 (s, 1H) 7.57-7.71 (m, 1H) 4.03 (q, J=7.09 Hz, 1H) 2.12 (s, 5H) 1.38 (s, 9H) ppm.

Step 2: Preparation of tert-butyl (3-ethynylbicyclo[1.1.1]pentan-1-yl)carbamate

The mixture of tert-butyl (3-formylbicyclo[1.1.1]pentan-1-yl)carbamate (1 g, 4.73 mmol, 1 eq), dimethyl (1-diazo-2-oxopropyl)phosphonate (1.00 g, 5.21 mmol, 1.1 eq) and K₂CO₃ (1.37 g, 9.94 mmol, 2.1 eq) in MeOH (10 mL) was stirred at 20° C. for 12 hours under N₂ atmosphere. The volatiles were then removed under reduced pressure. The resulting residue was dissolved in EtOAc (200 mL), washed with H₂O (100 mL). The organic layer was dried over Na₂SO₄, filtered and the filtrate was concentrated under reduced pressure. The desired tert-butyl (3-ethynylbicyclo[1.1.1]pentan-1-yl)carbamate (1.3 g, crude) was obtained as a yellow solid. 1H NMR (400 MHz, DMSO-d₆) δ 7.53-7.69 (m, 1H) 3.09 (s, 1H) 2.12 (s, 5H) 1.98 (s, 1H) 1.36 (s, 9H) ppm.

Step 3: Preparation of tert-butyl (3-((4-bromopyridin-2-yl)ethynyl)bicyclo[1.1.1]pentan-1-yl)carbamate

A mixture of tert-butyl (3-ethynylbicyclo[1.1.1]pentan-1-yl)carbamate (500 mg, 2.41 mmol, 1 eq), 2,4-dibromopyridine (685.75 mg, 2.89 mmol, 1.2 eq), Pd(PPh₃)₄ (278.76 mg, 0.241 mmol, 0.1 eq), TEA (488.20 mg, 4.82 mmol, 671.53 μL, 2 eq) and CuI (137.83 mg, 723.70 μmol, 0.3 eq) in DMF (10 mL) was stirred at 40° C. for 12 hours under N₂ atmosphere. After the completion of the reaction, the reaction mixture was cooled to room temperature and concentrated under reduced pressure. The resulting residue was dissolved in EtOAc (200 mL) and washed with H₂O (100 mL). The organic phase was dried over Na₂SO₄, filtered and the filtrate was concentrated under reduced pressure. The resulting residue was purified by column chromatography to afford tert-butyl (3-((4-bromopyridin-2-yl)ethynyl)bicyclo[1.1.1]pentan-1-yl)carbamate (546 mg, 62.3% yield) as a yellow solid. LCMS: 363.0 [M+H]⁺.

Step 4: Preparation of tert-butyl (3-((4-((1-(tert-butyl)-3-((1S,3R)-3-((tert-butyldimethylsilyl)oxy)cyclopentyl)-1H-pyrazol-5-yl)amino)pyridin-2-yl)ethynyl)bicyclo[1.1.1]pentan-1-yl)carbamate

A mixture of tert-butyl (3-((4-bromopyridin-2-yl)ethynyl)bicyclo[1.1.1]pentan-1-yl)carbamate (250 mg, 0.688 mmol, 1 eq), 1-(tert-butyl)-3-((1S,3R)-3-((tert-butyldimethylsilyl)oxy)cyclopentyl)-1H-pyrazol-5-amine (348.50 mg, 1.03 mmol, 1.5 eq), Xantphos (79.64 mg, 0.138 mmol, 0.2 eq), Pd₂(dba)₃ (63.02 mg, 0.0688 mmol, 0.1 eq) and Cs₂CO₃ (672.72 mg, 2.06 mmol, 3 eq) in dioxane (5 mL) was stirred at 90° C. for 12 hours under N₂ atmosphere. After the completion of the reaction, the reaction mixture was cooled to room temperature, filtered, rinsed with dioxane (10 mL) and the filtrate was concentrated under reduced pressure. The resulting residue was purified by column chromatography to produce tert-butyl (3-((4-((1-(tert-butyl)-3-((1S,3R)-3-((tert-butyldimethylsilyl)oxy)cyclopentyl)-1H-pyrazol-5-yl)amino)pyridin-2-yl)ethynyl)bicyclo[1.1.1]pentan-1-yl)carbamate (346 mg, 81.1% yield) as a yellow oil. LCMS: 620.5 [M+H]⁺.

Step 5: Preparation of tert-butyl (3-(2-(4-((1-(tert-butyl)-3-((1S,3R)-3-((tert-butyldimethylsilyl)oxy)cyclopentyl)-1H-pyrazol-5-yl)amino)pyridin-2-yl)ethyl)bicyclo[1.1.1]pentan-1-yl)carbamate

The mixture of tert-butyl (3-((4-((1-(tert-butyl)-3-((1S,3R)-3-((tert-butyldimethylsilyl)oxy)cyclopentyl)-1H-pyrazol-5-yl)amino)pyridin-2-yl)ethynyl)bicyclo[1.1.1]pentan-1-yl)carbamate (346 mg, 0.558 mmol, 1 eq) and Pt/C (5%, 500 mg) in THF (6 mL) was degassed and backfilled with H₂ for 3 times, and then stirred under H₂ (15 Psi) atmosphere at 20° C. for 1 hour. After the completion of the reaction, the solid was filtered off and rinsed with THF (5 mL). The filtrate was concentrated under reduced pressure to give tert-butyl (3-(2-(4-((1-(tert-butyl)-3-((1S,3R)-3-((tert-butyldimethylsilyl)oxy)cyclopentyl)-1H-pyrazol-5-yl)amino)pyridin-2-yl)ethyl)bicyclo[1.1.1]pentan-1-yl)carbamate (280 mg, crude) as a deep brown oil. LCMS: 624.4 [M+H]⁺.

Step 6: Preparation of tert-butyl (2-(2-(3-((tert-butoxycarbonyl)amino)bicyclo[1.1.1]pentan-1-yl)ethyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (3-(2-(4-((1-(tert-butyl)-3-((1S,3R)-3-((tert-butyldimethylsilyl)oxy)cyclopentyl)-1H-pyrazol-5-yl)amino)pyridin-2-yl)ethyl)bicyclo[1.1.1]pentan-1-yl)carbamate (200 mg, 0.321 mmol, 1 eq), Boc₂O (104.94 mg, 0.481 mmol, 110.46 μL, 1.5 eq) and DMAP (3.92 mg, 0.0321 mmol, 0.1 eq) in THF (6 mL) was added TEA (97.31 mg, 0.962 mmol, 133.85 μL, 3 eq), and the mixture was stirred at 40° C. for 2 hours under N₂ atmosphere. The reaction mixture was then cooled to room temperature and concentrated under reduced pressure. The remaining residue was purified by column chromatography to give tert-butyl (2-(2-(3-((tert-butoxycarbonyl)amino)bicyclo[1.1.1]pentan-1-yl)ethyl)pyridin-4-yl)(1-(tert-butyl)-3-((1S,3R)-3-((tert-butyldimethylsilyl)oxy)cyclopentyl)-1H-pyrazol-5-yl)carbamate (180 mg, 77.5% yield) as a red oil. LCMS: 724.5 [M+H]⁺.

Step 7: Preparation of tert-butyl (2-(2-(3-((tert-butoxycarbonyl)amino)bicyclo[1.1.1]pentan-1-yl)ethyl)pyridin-4-yl)(1-(tert-butyl)-3-((1S,3R)-3-hydroxycyclopentyl)-1H-pyrazol-5-yl)carbamate

The mixture of tert-butyl (2-(2-(3-((tert-butoxycarbonyl)amino)bicyclo[1.1.1]pentan-1-yl)ethyl)pyridin-4-yl)(1-(tert-butyl)-3-((1S,3R)-3-((tert-butyldimethylsilyl)oxy)cyclopentyl)-1H-pyrazol-5-yl)carbamate (170 mg, 0.235 mmol, 1 eq) and TBAF (245.55 mg, 0.939 mmol, 4 eq) in THF (3 mL) was stirred at 20° C. for 12 hours. The reaction mixture was then concentrated under reduced pressure and the resulting residue was dissolved in 100 mL of EtOAc. The organic phase was washed with water (30 mL), dried over Na₂SO₄, filtered, and the filtrate was concentrated under reduced pressure. The desired tert-butyl (2-(2-(3-((tert-butoxycarbonyl)amino)bicyclo[1.1.1]pentan-1-yl)ethyl)pyridin-4-yl)(1-(tert-butyl)-3-((1S,3R)-3-hydroxycyclopentyl)-1H-pyrazol-5-yl)carbamate (210 mg, crude) was obtained as a yellow oil. LCMS: 610.3 [M+H]⁺.

Step 8: Preparation of tert-butyl (2-(2-(3-((tert-butoxycarbonyl)amino)bicyclo[1.1.1]pentan-1-yl)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(2-(3-((tert-butoxycarbonyl)amino)bicyclo[1.1.1]pentan-1-yl)ethyl)pyridin-4-yl)(1-(tert-butyl)-3-((1S,3R)-3-hydroxycyclopentyl)-1H-pyrazol-5-yl)carbamate (200 mg, 0.328 mmol, 1 eq), Py (129.72 mg, 1.64 mmol, 132.36 μL, 5 eq) and DMAP (20.03 mg, 0.164 mmol, 0.5 eq) in DCM (4 mL) was added (4-nitrophenyl) carbonochloridate (198.33 mg, 0.984 mmol, 3 eq), and the mixture was stirred at 20° C. for 12 hours under N₂ atmosphere. The reaction mixture was then concentrated under reduced pressure. The resulting residue was purified by column chromatography to yield tert-butyl (2-(2-(3-((tert-butoxycarbonyl)amino)bicyclo[1.1.1]pentan-1-yl)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate (200 mg, 52.5% yield) as a white solid. LCMS: 775.4 [M+H]⁺.

Step 9: Preparation of (1R,3S)-3-(5-((2-(2-(3-aminobicyclo[1.1.1]pentan-1-yl)ethyl)pyridin-4-yl)amino)-1-(tert-butyl)-1H-pyrazol-3-yl)cyclopentyl (4-nitrophenyl) carbonate

The solution of tert-butyl (2-(2-(3-((tert-butoxycarbonyl)amino)bicyclo[1.1.1]pentan-1-yl)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate (71 mg, 0.0916 mmol, 1 eq) and TFA (1.54 g, 13.46 mmol, 1 mL, 146.93 eq) in DCM (3 mL) was stirred at 20° C. for 7 hours under N₂ atmosphere. The volatiles were evaporated under reduced pressure to give (1R,3S)-3-(5-((2-(2-(3-aminobicyclo[1.1.1]pentan-1-yl)ethyl)pyridin-4-yl)amino)-1-(tert-butyl)-1H-pyrazol-3-yl)cyclopentyl (4-nitrophenyl) carbonate (118 mg, crude) as a yellow oil. LCMS: 575.3 [M+H]⁺.

Step 10: Preparation of (1¹S,1³R,Z)-2¹-(tert-butyl)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one

To a solution of (1R,3S)-3-(5-((2-(2-(3-aminobicyclo[1.1.1]pentan-1-yl)ethyl)pyridin-4-yl)amino)-1-(tert-butyl)-1H-pyrazol-3-yl)cyclopentyl (4-nitrophenyl) carbonate (113 mg, 0.197 mmol, 1 eq) in DMSO (56 mL) was added DIEA (1.06 g, 8.17 mmol, 1.42 mL, 41.57 eq), and the mixture was stirred at 100° C. for 2 hours under N₂ atmosphere. The mixture was then cooled to room temperature, and the volatiles were removed under reduced pressure. The remaining residue was dissolved in 100 mL of ethyl acetate, washed with 30 mL of water. The organic phase was dried over Na₂SO₄, filtered and the filtrate was concentrated under reduced pressure to give (1¹S,1³R,Z)-2¹-(tert-butyl)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one (60 mg, crude) as a yellow oil. LCMS: 436.3 [M+H]⁺.

Step 11: Preparation of (1¹S,1³R,Z)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one

A solution of (1¹S,1³R,Z)-2¹-(tert-butyl)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one (50 mg, 0.115 mmol, 1 eq) in formic acid (1 mL) was stirred at 100° C. for 2 hours under N₂ atmosphere. The mixture was then cooled to room temperature and the volatiles were evaporated under reduced pressure. The crude product was purified by Prep-HPLC (column: Phenomenex Luna C18 150*25 mm*10 um; mobile phase: [water (NH₃·H₂O)-ACN]; B %: 10%-90%, 20 min) to give (1¹S,1³R,Z)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one (11.49 mg, 25.4% yield) as a white solid. LCMS: 380.3 [M+H]⁺; ¹H NMR (400 MHz, DMSO-d₆) δ 12.08-12.16 (m, 1H), 8.63 (s, 1H), 8.03 (d, J=5.62 Hz, 1H), 7.40 (s, 1H), 6.75 (d, J=1.83 Hz, 1H), 6.55 (dd, J=5.56, 2.02 Hz, 1H), 6.00 (s, 1H), 5.13 (br d, J=5.14 Hz, 1H), 3.17-3.24 (m, 1H), 2.54 (br d, J=6.97 Hz, 2H), 1.93-2.02 (m, 1H), 1.86-1.91 (m, 2H), 1.68-1.83 (m, 11H) ppm.

Example 12: synthesis of (1¹S,1³R,Z)-4⁶-(difluoromethyl)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one (compound 20)

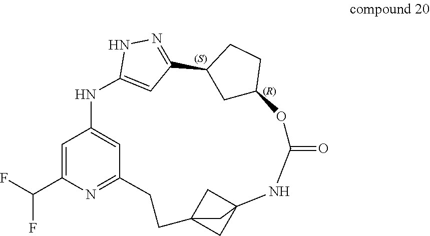

Step 1: Preparation of 2,4-dibromo-6-(difluoromethyl)pyridine

The solution of 4-bromo-2-(difluoromethyl)pyridine 1-oxide (1.5 g, 6.70 mmol, 1 eq) in POBr₃ (15 mL) was stirred at 100° C. for 1 hour under N₂ atmosphere. After the reaction was completed, the mixture was cooled to room temperature and carefully poured into saturated aqueous Na₂CO₃ (30 mL). The mixture was extracted with EtOAc (50 mL×2). The combined organic layers were washed with brine (30 mL), dried over Na₂SO₄, filtered, and the filtrate was concentrated to give a residue, which was purified by column chromatography. The desired 2,4-dibromo-6-(difluoromethyl)pyridine (582 mg, 30.4% yield) was obtained as a yellow solid. ¹HNMR (400 MHz, DMSO-d₆) δ 9.58 (s, 1H), 7.64-7.59 (m, 1H), 4.05-4.00 (m, 1H), 2.11 (s, 5H), 1.37 (s, 9H) ppm.

Step 2: Preparation of tert-butyl (3-((4-bromo-6-(difluoromethyl)pyridin-2-yl)ethynyl)bicyclo[1.1.1]pentan-1-yl)carbamate

A mixture of tert-butyl (3-ethynylbicyclo[1.1.1]pentan-1-yl)carbamate (350 mg, 1.69 mmol, 1 eq), 2,4-dibromo-6-(difluoromethyl)pyridine (581.36 mg, 2.03 mmol, 1.2 eq), CuI (96.48 mg, 0.50 mmol, 0.3 eq), TEA (341.75 mg, 3.38 mmol, 2 eq) and Pd(PPh₃)₄ (195.13 mg, 0.16 mmol, 0.1 eq) in DMF (10 mL) was stirred at 40° C. for 12 hours under N₂ atmosphere. After the completion of the reaction, the mixture was cooled to room temperature, diluted with H₂O (100 mL) and extracted with EtOAc (100 mL×2). The combined organic layers were washed with brine (50 mL), dried over Na₂SO₄, filtered, and the filtrate was concentrated under reduced pressure. The resulting residue was purified by pre-TLC (PE:EA=3:1) to give tert-butyl (3-((4-bromo-6-(difluoromethyl)pyridin-2-yl)ethynyl)bicyclo[1.1.1]pentan-1-yl)carbamate (400 mg, 57.3% yield) as a yellow solid. LCMS: 413.1, 415.1 [M+H]⁺.

Step 3: Preparation of tert-butyl (3-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)ethynyl)bicyclo[1.1.1]pentan-1-yl)carbamate

A mixture of tert-butyl (3-((4-bromo-6-(difluoromethyl)pyridin-2-yl)ethynyl)bicyclo[1.1.1]pentan-1-yl)carbamate (400 mg, 0.96 mmol, 1 eq), 1-(tert-butyl)-3-((1S,3R)-3-((tert-butyldimethylsilyl)oxy)cyclopentyl)-1H-pyrazol-5-amine (392.09 mg, 1.15 mmol, 1.2 eq), Xantphos (112.01 mg, 0.18 mmol, 0.2 eq), Pd₂(dba)₃ (88.63 mg, 0.09 mmol, 0.1 eq) and Cs₂CO₃ (946.10 mg, 2.90 mmol, 3 eq) in dioxane (8 mL) was stirred at 90° C. for 1 hour under N₂ atmosphere. After the reaction was completed, the reaction was cooled to room temperature. The mixture was filtered through a pad of celite, rinsed with ethyl acetate (30 ml). The filtrate was concentrated under reduced pressure to give a residue, which was purified by column chromatography. The desired tert-butyl (3-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)ethynyl)bicyclo[1.1.1]pentan-1-yl)carbamate (613 mg, 94.6% yield) was obtained as a yellow solid. LCMS: 670.3 [M+H]⁺.

Step 4: Preparation of tert-butyl (3-(2-(4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)ethyl)bicyclo[1.1.1]pentan-1-yl)carbamate

The mixture of tert-butyl (3-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)ethynyl)bicyclo[1.1.1]pentan-1-yl)carbamate (520 mg, 0.77 mmol, 1 eq) and Pt/C (5%, 700 mg) in THF (10 mL) was degassed and backfilled with H₂ for 3 times, and then stirred under H₂ (15 Psi) at 25° C. for 1 hour. After the reaction was completed, the reaction mixture was filtered through a pad of celite, rinsed with THF (10 mL). The combined filtrate was concentrated under reduced pressure to give tert-butyl (3-(2-(4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)ethyl)bicyclo[1.1.1]pentan-1-yl)carbamate (515 mg, 0.76 mmol) as a yellow oil, which was used in the next step without further purification. LCMS: 674.5 [M+H]⁺.

Step 5: Preparation of tert-butyl (2-(2-(3-((tert-butoxycarbonyl)amino)bicyclo[1.1.1]pentan-1-yl)ethyl)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (3-(2-(4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)ethyl)bicyclo[1.1.1]pentan-1-yl)carbamate (515 mg, 0.76 mmol, 1 eq) in DCM (12 mL) was added Boc₂O (153.01 mg, 1.14 mmol, 0.26 mL, 1.5 eq), DMAP (9.40 mg, 0.07 mmol, 0.1 eq) and TEA (141.89 mg, 2.29 mmol, 0.32 mL, 3 eq), and the mixture was stirred at 25° C. for 1 hour under N₂ atmosphere. After the reaction was completed, the volatiles were removed in vacuo. The resulting residue was purified by column chromatography to give tert-butyl (2-(2-(3-((tert-butoxycarbonyl)amino)bicyclo[1.1.1]pentan-1-yl)ethyl)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate (382 mg, 64.6% yield) as a yellow oil. LCMS: 774.4 [M+H]⁺.

Step 6: Preparation of tert-butyl (2-(2-(3-((tert-butoxycarbonyl)amino)bicyclo[1.1.1]pentan-1-yl)ethyl)-6-(difluoromethyl)pyridin-4-yl)(1-(tert-butyl)-3-((1S,3R)-3-hydroxycyclopentyl)-1H-pyrazol-5-yl)carbamate

To a solution of tert-butyl (2-(2-(3-((tert-butoxycarbonyl)amino)bicyclo[1.1.1]pentan-1-yl)ethyl)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate (382 mg, 0.49 mmol, 1 eq) in THF (8.5 mL) was added TBAF (457.5 mg, 1.75 mmol, 4 eq), and the mixture was stirred at 25° C. for 7 hours under N₂ atmosphere. After the completion of the reaction, the mixture was quenched with H₂O (50 mL) and extracted with EtOAc (50 mL×2). The combined organic layers were washed with brine (50 mL×2), dried over anhydrous Na₂SO₄, filtered, and the filtrate was concentrated in vacuo. The crude tert-butyl (2-(2-(3-((tert-butoxycarbonyl)amino)bicyclo[1.1.1]pentan-1-yl)ethyl)-6-(difluoromethyl)pyridin-4-yl)(1-(tert-butyl)-3-((1S,3R)-3-hydroxycyclopentyl)-1H-pyrazol-5-yl)carbamate (330 mg) was obtained as a yellow oil, which was used into the next step without further purification. LCMS: 660.3 [M+H]⁺.

Step 7: Preparation of tert-butyl (2-(2-(3-((tert-butoxycarbonyl)amino)bicyclo[1.1.1]pentan-1-yl)ethyl)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(2-(3-((tert-butoxycarbonyl)amino)bicyclo[1.1.1]pentan-1-yl)ethyl)-6-(difluoromethyl)pyridin-4-yl)(1-(tert-butyl)-3-((1S,3R)-3-hydroxycyclopentyl)-1H-pyrazol-5-yl)carbamate (330 mg, 0.50 mmol, 1 eq) in DCM (7 mL) was added (4-nitrophenyl) carbonochloridate (302.44 mg, 1.50 mmol, 3 eq), DMAP (30.55 mg, 0.25 mmol, 0.5 eq) and Pyridine (237.37 mg, 3.00 mmol, 0.24 mL, 6 eq), and the mixture was stirred at 25° C. for 12 hours under N₂ atmosphere. After the completion of the reaction, the mixture was filtered, and the filtrate was concentrated in vacuo. The resulting residue was purified by column chromatography to give tert-butyl (2-(2-(3-((tert-butoxycarbonyl)amino)bicyclo[1.1.1]pentan-1-yl)ethyl)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate (174 mg, 0.21 mmol, 42.2% yield) as a yellow oil. LCMS: 825.4 [M+H]⁺.

Step 8: Preparation of (1R,3S)-3-(5-((2-(2-(3-aminobicyclo[1.1.1]pentan-1-yl)ethyl)-6-(difluoromethyl)pyridin-4-yl)amino)-1-(tert-butyl)-1H-pyrazol-3-yl)cyclopentyl (4-nitrophenyl) carbonate

To a solution of tert-butyl (2-(2-(3-((tert-butoxycarbonyl)amino)bicyclo[1.1.1]pentan-1-yl)ethyl)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate (164 mg, 0.19 mmol, 1 eq) in DCM (7.3 mL) was added TFA (3.57 g, 31.35 mmol, 2.33 mL, 157.69 eq), and the mixture was stirred at 25° C. for 12 hours under N₂ atmosphere. After the reaction was completed, the mixture was concentrated in vacuo to afford crude (1R,3S)-3-(5-((2-(2-(3-aminobicyclo[1.1.1]pentan-1-yl)ethyl)-6-(difluoromethyl)pyridin-4-yl)amino)-1-(tert-butyl)-1H-pyrazol-3-yl)cyclopentyl (4-nitrophenyl) carbonate (260 mg, 0.42 mmol) as a yellow oil, which was used into the next step without further purification. LCMS: 625.3 [M+H]⁺.

Step 9: Preparation of (1¹S,1³R,Z)-2¹-(tert-butyl)-4⁶-(difluoromethyl)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one

To a solution of (1R,3S)-3-(5-((2-(2-(3-aminobicyclo[1.1.1]pentan-1-yl)ethyl)-6-(difluoromethyl)pyridin-4-yl)amino)-1-(tert-butyl)-1H-pyrazol-3-yl)cyclopentyl (4-nitrophenyl) carbonate (260 mg, 0.41 mmol, 1 eq) in DMSO (130 mL) was added DIEA (0.53 g, 4.16 mmol, 0.72 mL, 10 eq), and the mixture was stirred at 100° C. for 1 hour under N₂ atmosphere. After the reaction was completed, the mixture was cooled to room temperature, diluted with H₂O (200 mL) and extracted with EtOAc (200 mL×2). The combined organic layers were washed with brine (50 mL), dried over Na₂SO₄, filtered, and the filtrate was concentrated under reduced pressure. The crude (1¹S,1³R,Z)-2¹-(tert-butyl)-4⁶-(difluoromethyl)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one (90 mg, 0.18 mmol) was obtained as a yellow oil. LCMS: 486.3 [M+H]⁺.

Step 10: Preparation of (1¹S,1³R,Z)-4⁶-(difluoromethyl)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one

The solution of (1¹S,1³R,Z)-2¹-(tert-butyl)-4⁶-(difluoromethyl)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one (90 mg, 0.18 mmol, 1 eq) in formic acid (4 mL) was stirred at 100° C. for 0.5 hour under N₂ atmosphere. After the reaction was completed, the mixture was cooled to room temperature and concentrated in vacuo. The resulting residue was purified by prep-HPLC (column: Phenomenex Luna C18 150×25 mm×10 um; mobile phase: [water (NH₃·H₂O)-ACN]; B %: 10%-90%, 20 min) to afford (1¹S,1³R,Z)-4⁶-(difluoromethyl)-2¹H-10-oxa-3,8-diaza-4(4,2)-pyridina-2(3,5)-pyrazola-7(1,3)-bicyclo[1.1.1]pentana-1(1,3)-cyclopentanacyclodecaphan-9-one (33.53 mg, 40.7% yield) as a white solid. LCMS: 430.2 [M+H]⁺; ¹H NMR (400 MHz, DMSO-d₆) δ 12.22 (br s, 1H), 8.97 (s, 1H), 7.41 (s, 1H), 6.90-6.54 (m, 3H), 6.03 (s, 1H), 5.14-5.12 (m, 1H), 3.40-3.35 (m, 1H), 3.26-3.17 (m, 1H), 2.60 (br d, J=8.0 Hz, 2H), 1.99 (br dd, J=6.6, 11.7 Hz, 1H), 1.88 (br d, J=8.8 Hz, 2H), 1.82 (br s, 2H), 1.78 (br d, J=4.0 Hz, 1H), 1.77-1.68 (m, 7H) ppm.

Example 13: (1¹S,1³R,Z)-4⁶-(difluoromethyl)-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one (compound 23)

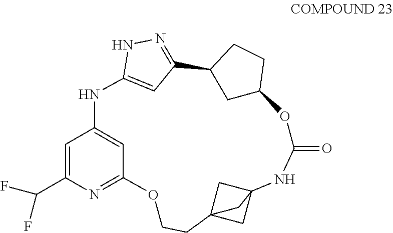 [SMILES: O=C1NC23CC(CCOc4cc(cc(C(F)F)n4)Nc4cc(n[nH]4)[C@H]4CC[C@H](C4)O1)(C2)C3]

Step 1: Preparation of tert-butyl (3-(2-((4-bromo-6-(difluoromethyl)pyridin-2-yl)oxy)ethyl)bicyclo[1.1.1]pentan-1-yl)carbamate

To a solution of 4-bromo-6-(difluoromethyl)pyridin-2-ol (125 mg, 0.56 mmol, 1 eq) and tert-butyl (3-(2-hydroxyethyl)bicyclo[1.1.1]pentan-1-yl)carbamate (152.21 mg, 0.67 mmol, 1.2 eq) in toluene (3 mL) was added 2-(tributyl-phosphanylidene)acetonitrile (202.02 mg, 0.84 mmol, 1.5 eq) and the mixture was stirred at 80° C. for 12 hours under N₂ atmosphere. After the reaction was completed, the mixture was cooled to room temperature and concentrated under reduced pressure. The resulting residue was purified by column chromatography to give tert-butyl (3-(2-((4-bromo-6-(difluoromethyl)pyridin-2-yl)oxy)ethyl)bicyclo[1.1.1]pentan-1-yl)carbamate (200 mg, 721.9% yield) as a yellow oil. LCMS: 376.7 [M+H]⁺.

Step 2: Preparation of tert-butyl (3-(2-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)oxy)ethyl)bicyclo[1.1.1]pentan-1-yl)carbamate

A mixture of tert-butyl (3-(2-((4-bromo-6-(difluoromethyl)pyridin-2-yl)oxy)ethyl)bicyclo[1.1.1]pentan-1-yl)carbamate (185 mg, 0.43 mmol, 1 eq), 1-(tert-butyl)-3-((1S,3R)-3-((tert-butyldimethylsilyl)oxy)cyclopentyl)-1H-pyrazol-5-amine (144.13 mg, 0.43 mmol, 1 eq), Pd₂(dba)₃ (39.10 mg, 0.042 mmol, 0.1 eq), Xantphos (37.06 mg, 0.064 mmol, 0.15 eq) and Cs₂CO₃ (417.35 mg, 1.28 mmol, 3 eq) in dioxane (6 mL) was stirred at 80° C. for 6 hours under N₂ atmosphere. After the reaction was completed, the mixture was cooled to room temperature, poured into water (10 mL) and extracted with EtOAc (10 mL×3). The combined organic layers were washed with brine (30 mL), dried over Na₂SO₄, filtered and the filtrate was evaporated under reduced pressure. The resulting crude product was purified by Prep-TLC (PE:EA=3:1) to give tert-butyl (3-(2-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)oxy)ethyl)bicyclo[1.1.1]pentan-1-yl)carbamate (230 mg, 74.2% yield) as an orange solid. LCMS: 690.4 [M+H]⁺; ¹H NMR (400 MHz, CHLOROFORM-d) δ=6.52-6.18 (m, 2H), 5.97 (s, 1H), 5.87 (d, J=1.0 Hz, 1H), 5.33 (s, 1H), 4.20 (t, J=6.6 Hz, 2H), 2.99 (t, J=8.4 Hz, 1H), 2.34-2.21 (m, 1H), 1.94-1.85 (m, 8H), 1.82-1.74 (m, 2H), 1.67-1.55 (m, 4H), 1.50 (s, 9H), 1.38 (s, 9H), 0.82 (s, 9H), 0.05 (d, J=3.1 Hz, 6H) ppm.

Step 3: Preparation of tert-butyl (2-(2-(3-((tert-butoxycarbonyl)amino)bicyclo[1.1.1]pentan-1-yl)ethoxy)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (3-(2-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)oxy)ethyl)bicyclo[1.1.1]pentan-1-yl)carbamate (220 mg, 0.32 mmol, 1 eq) in THF (6 mL) was added Boc₂O (139.18 mg, 0.64 mmol, 2 eq), DMAP (38.96 mg, 0.32 mmol, 1 eq) and TEA (96.80 mg, 0.95 mmol, 3 eq), and the mixture was stirred at 25° C. for 2 hours under N₂ atmosphere. The reaction mixture was then concentrated under reduced pressure. The resulting residue was purified by prep-TLC (Petroleum ether: Ethyl acetate=3:1) to give tert-butyl (2-(2-(3-((tert-butoxycarbonyl)amino)bicyclo[1.1.1]pentan-1-yl)ethoxy)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate (230 mg, 83.1% yield) as a yellow oil. LCMS: 791.6 [M+H]⁺.

Step 4: Preparation of tert-butyl (2-(2-(3-((tert-butoxycarbonyl)amino)bicyclo[1.1.1]pentan-1-yl)ethoxy)-6-(difluoromethyl)pyridin-4-yl)(1-(tert-butyl)-3-((1S,3R)-3-hydroxycyclopentyl)-1H-pyrazol-5-yl)carbamate

The solution of tert-butyl (2-(2-(3-((tert-butoxycarbonyl)amino)bicyclo[1.1.1]pentan-1-yl)ethoxy)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate (210 mg, 0.26 mmol, 1 eq) and TBAF (1 M, 1.06 mL, 4 eq) in THF (3 mL) was stirred at 25° C. for 12 hours. The reaction mixture was then concentrated under reduced pressure. The resulting residue was purified by column chromatography to afford tert-butyl (2-(2-(3-((tert-butoxycarbonyl)amino)bicyclo[1.1.1]pentan-1-yl)ethoxy)-6-(difluoromethyl)pyridin-4-yl)(1-(tert-butyl)-3-((1S,3R)-3-hydroxycyclopentyl)-1H-pyrazol-5-yl)carbamate (130 mg, 65.1% yield) as a yellow oil. LCMS: 676.5 [M+H]⁺.

Step 5: Preparation of tert-butyl (2-(2-(3-((tert-butoxycarbonyl)amino)bicyclo[1.1.1]pentan-1-yl)ethoxy)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(2-(3-((tert-butoxycarbonyl)amino)bicyclo[1.1.1]pentan-1-yl)ethoxy)-6-(difluoromethyl)pyridin-4-yl)(1-(tert-butyl)-3-((1S,3R)-3-hydroxycyclopentyl)-1H-pyrazol-5-yl)carbamate (110 mg, 0.16 mmol, 1 eq) in THF (2 mL) was added pyridine (64.38 mg, 0.81 mmol, 5 eq) and DMAP (9.94 mg, 0.08 mmol, 0.5 eq), followed by the addition of the solution of (4-nitrophenyl) carbonochloridate (98.43 mg, 0.488 mmol, 3 eq) in DCM (2 mL) at 0° C. under N₂ atmosphere. After the completion of the addition, the mixture was stirred at 20° C. for 3 hours under N₂ atmosphere and the volatiles were then removed under reduced pressure. The resulting residue was purified by prep-TLC (Petroleum ether: Ethyl acetate=3:1) to give tert-butyl (2-(2-(3-((tert-butoxycarbonyl)amino)bicyclo[1.1.1]pentan-1-yl)ethoxy)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate (130 mg, 64.6% yield) as a yellow oil. LCMS: 841.5 [M+H]⁺.

Step 6: Preparation of (1R,3S)-3-(5-((2-(2-(3-aminobicyclo[1.1.1]pentan-1-yl)ethoxy)-6-(difluoromethyl)pyridin-4-yl)amino)-1-(tert-butyl)-1H-pyrazol-3-yl)cyclopentyl (4-nitrophenyl) carbonate

The solution of tert-butyl (2-(2-(3-((tert-butoxycarbonyl)amino)bicyclo[1.1.1]pentan-1-yl)ethoxy)-6-(difluoromethyl)pyridin-4-yl)(1-(tert-butyl)-3-((1S,3R)-3-(((4-nitrophenoxy)carbonyl)oxy)cyclopentyl)-1H-pyrazol-5-yl)carbamate (120 mg, 0.14 mmol, 1 eq) and TFA (1.54 g, 13.46 mmol, 94.34 eq) in DCM (4 mL) was stirred at 20° C. for 2 hours. After the completion of the reaction, the reaction mixture was concentrated under reduced pressure to give the crude (1R,3S)-3-(5-((2-(2-(3-aminobicyclo[1.1.1]pentan-1-yl)ethoxy)-6-(difluoromethyl)pyridin-4-yl)amino)-1-(tert-butyl)-1H-pyrazol-3-yl)cyclopentyl (4-nitrophenyl) carbonate (90 mg) as a yellow oil, which was used in the next step without further purification. LCMS: 641.4 [M+H]⁺.

Step 7: Preparation of (1¹S,1³R,Z)-2¹-(tert-butyl)-4⁶-(difluoromethyl)-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one

To a solution of (1R,3S)-3-(5-((2-(2-(3-aminobicyclo[1.1.1]pentan-1-yl)ethoxy)-6-(difluoromethyl)pyridin-4-yl)amino)-1-(tert-butyl)-1H-pyrazol-3-yl)cyclopentyl (4-nitrophenyl) carbonate (80 mg, 0.12 mmol, 1 eq) in DMF (10 mL) was added TEA (252.71 mg, 2.50 mmol, 20 eq), and the mixture was stirred at 100° C. for 0.5 hour under N₂ atmosphere. After the starting material was consumed, the mixture was cooled to room temperature and concentrated under reduced pressure. The resulting residue was purified by prep-TLC (Petroleum ether: Ethyl acetate=1:1) to give (1¹S,1³R,Z)-2¹-(tert-butyl)-4⁶-(difluoromethyl)-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one (80 mg, 0.16 mmol) as a yellow oil, which was used in the next step without further purification. LCMS: 502.4 [M+H]⁺.

Step 8: Preparation (1¹S,1³R,Z)-4⁶-(difluoromethyl)-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one

The solution of (1¹S,1³R,Z)-2¹-(tert-butyl)-4⁶-(difluoromethyl)-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one (70 mg, 0.14 mmol, 1 eq) in formic acid (1 mL) was stirred at 100° C. for 0.5 hour under N₂ atmosphere. After the reaction was completed, the mixture was cooled to room temperature, and concentrated under reduced pressure. The resulting residue was purified by prep-HPLC (column: YMC Triart C18 150*25 mm*5 um; mobile phase: [water(FA)-ACN]; gradient:34%-64% B over 10 min) to afford (1¹S,1³R,Z)-4⁶-(difluoromethyl)-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one (33.06 mg, 53.1% yield) as a white solid. LCMS: 446.3 [M+H]⁺; ¹HNMR (400 MHz, DMSO-d₆) 5=12.36-12.13 (m, 1H), 9.13 (s, 1H), 7.43 (s, 1H), 6.82-6.54 (m, 2H), 6.34 (s, 1H), 6.03 (s, 1H), 5.05-5.14 (m, 1H), 4.29-4.18 (m, 2H), 3.21-3.08 (m, 1H), 2.64-2.52 (m, 1H), 1.97-1.90 (m, 1H), 1.88-1.73 (m, 10H), 1.71-1.60 (m, 2H) ppm.

Example 14: Synthesis of (1'S,3′R,6′R,Z)-6′-methylspiro[cyclopropane-1 9′-5,12-dioxa-3,10-diaza-4(4,2)-pyridina-2(3,5)-pyrazola-1(1,3)-cyclopentanacyclododecaphan]-11′-one (compound 109) and (1'S,3′R,6'S,Z)-6′-methylspiro[cyclopropane-1 9′-5,12-dioxa-3,10-diaza-4(4,2)-pyridina-2(3,5)-pyrazola-1(1,3)-cyclopentanacyclododecaphan]-11′-one(compound 110)

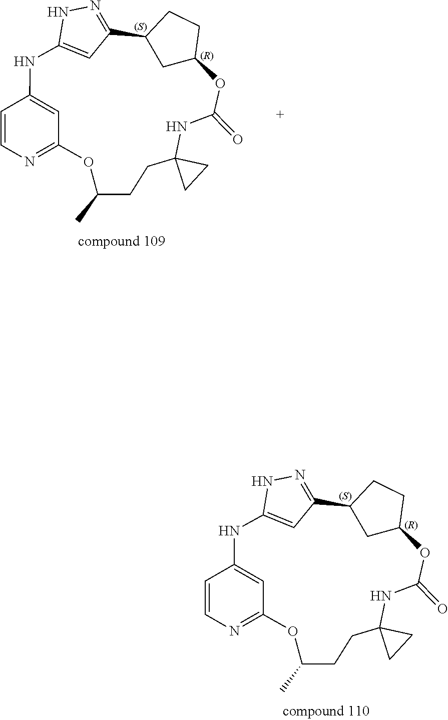

Step 1: Preparation of ethyl (E)-3-(1-((tert-butoxycarbonyl)amino)cyclopropyl)acrylate

The mixture of tert-butyl (1-formylcyclopropyl)carbamate (1.7 g, 9.18 mmol, 1 eq) and ethyl 2-(triphenyl-phosphanylidene)acetate (3.52 g, 10.10 mmol, 1.1 eq) in DCM (15 mL) was stirred at 20° C. for 12 hours under N₂ atmosphere. After the reaction was completed, the mixture was filtered and rinsed with DCM (30 mL). The combined filtrate was concentrated to give a residue, which was purified by column chromatography. The desired ethyl (E)-3-(1-((tert-butoxycarbonyl)amino)cyclopropyl)acrylate (2.2 g, 58.5% yield) was obtained as a yellow oil. LCMS: 156.2 [M+H-100]⁺.

Step 2: Preparation of ethyl 3-(1-((tert-butoxycarbonyl)amino)cyclopropyl)propanoate

To a solution of ethyl (E)-3-(1-((tert-butoxycarbonyl)amino)cyclopropyl)acrylate (2.2 g, 8.62 mmol, 1 eq) in EtOAc (50 mL) was added Pd/C (140 mg, 10% purity). The mixture was degassed and backfilled with H₂ for 3 times, and then stirred under H₂ atmosphere (15 psi) at 20° C. for 2 hours. The reaction mixture was then filtered through a short pad of celite and rinsed with ethyl acetate (50 mL). The combined filtrate was concentrated under reduced pressure to give ethyl 3-(1-((tert-butoxycarbonyl)amino)cyclopropyl)propanoate (2.0 g, 72.2% yield) as a yellow oil. ¹HNMR (400 MHz, CHLOROFORM-d) δ 4.22-4.16 (m, 2H), 2.52-2.41 (m, 2H), 1.93 (br s, 2H), 1.50 (br s, 9H), 1.34-1.31 (m, 3H), 0.82 (br s, 2H), 0.70 (br s, 2H) ppm.

Step 3: Preparation of 3-(1-((tert-butoxycarbonyl)amino)cyclopropyl)propanoic acid

To a solution of ethyl 3-(1-((tert-butoxycarbonyl)amino)cyclopropyl)propanoate (1.0 g, 3.89 mmol, 1 eq) in THF (20 mL) and H₂O (20 mL) was added LiOH·H₂O (652.30 mg, 15.54 mmol, 4 eq), and the mixture was stirred for 16 hours at 25° C. The mixture was then acidified with 0.5N HCl to pH=3 and extracted with EtOAc (20 mL×2). The combined organic phase was dried over Na₂SO₄, filtered, and the filtrate was evaporated under reduced pressure to give 3-(1-((tert-butoxycarbonyl)amino)cyclopropyl)propanoic acid (880 mg, 1.74 mmol) as yellow oil, which was used in the next step without further purification. LCMS: 251.9 [M+Na]⁺.

Step 4: Preparation of tert-butyl (1-(3-(methoxy(methyl)amino)-3-oxopropyl)cyclopropyl)carbamate

To a solution 3-(1-((tert-butoxycarbonyl)amino)cyclopropyl)propanoic acid (850 mg, 3.71 mmol, 1 eq) in DCM (20 mL) was added DIEA (1.44 g, 11.12 mmol, 1.94 mL, 3 eq), HATU (2.11 g, 5.56 mmol, 1.5 eq) and N-methoxymethanamine hydrochloride (542.45 mg, 5.56 mmol, 1.5 eq), and the mixture was stirred at 20° C. for 12 hours until the completion of the reaction. The mixture was poured into saturated aqueous NaHCO₃ (20 mL) and extracted with EtOAc (20 mL×3). The combined organic phase was washed with brine (50 mL), dried over Na₂SO₄, filtered and the filtrate was concentrated.

The resulting crude product was purified by column chromatography to afford the desired tert-butyl (1-(3-(methoxy(methyl)amino)-3-oxopropyl)cyclopropyl)carbamate (1.2 g, 83.2% yield) as a yellow oil. ¹HNMR (400 MHz, CHLOROFORM-d) δ 3.65-3.59 (m, 3H), 3.11 (s, 3H), 2.55-2.38 (m, 2H), 1.85-1.70 (m, 2H), 1.36 (br s, 9H), 0.69 (br s, 2H), 0.57 (br s, 2H) ppm.

Step 5: Preparation of tert-butyl (1-(3-oxobutyl)cyclopropyl)carbamate

To a solution of tert-butyl (1-(3-(methoxy(methyl)amino)-3-oxopropyl)cyclopropyl)carbamate (1.1 g, 4.04 mmol, 1 eq) in THF (20 mL) was added MeMgBr (3 M, 4.04 mL, 3 eq) slowly at 0° C. After the completion of the addition, the mixture was kept stirring at 0° C. for 1 hour. The reaction was then quenched with saturated aqueous NH₄Cl (20 mL). The mixture was extracted with EtOAc (20 mL×3). The combined organic phase was dried over Na₂SO₄, filtered, and the filtrate was concentrated. The resulting crude was purified by column chromatography to afford the desired tert-butyl (1-(3-oxobutyl)cyclopropyl)carbamate (450 mg, 44.1% yield) as a yellow oil. ¹HNMR (400 MHz, CHLOROFORM-d) δ 2.53 (t, J=7.6 Hz, 2H), 2.09 (s, 3H), 1.73 (br t, J=6.7 Hz, 2H), 1.36 (br d, J=4.4 Hz, 9H), 0.69 (br s, 2H), 0.60-0.50 (m, 2H) ppm.

Step 6: Preparation of tert-butyl (1-(3-hydroxybutyl)cyclopropyl)carbamate

To a solution of tert-butyl (1-(3-oxobutyl)cyclopropyl)carbamate (450 mg, 1.98 mmol, 1 eq) in MeOH (10 mL) was added NaBH₄ (89.88 mg, 2.38 mmol, 1.2 eq) in portions at 0° C. under N₂ atmosphere. After the completion of the addition, the reaction mixture was stirred at 0° C. for 2 hours until the completion of the reaction. The reaction mixture was quenched with saturated aqueous NH₄Cl (10 mL) at 0° C., and then extracted with EtOAc (10 mL×3). The combined organic layers were dried over Na₂SO₄, filtered and the filtrate was concentrated under reduced pressure. The resulting residue was purified by column chromatography to give tert-butyl (1-(3-hydroxybutyl)cyclopropyl)carbamate (300 mg, 59.5% yield) as a yellow oil. ¹HNMR (400 MHz, METHANOL-d₄) δ 4.93-4.86 (m, 3H), 3.83-3.69 (m, 1H), 3.34 (br s, 2H), 1.53-1.39 (m, 9H), 1.24-1.10 (m, 2H), 0.69 (br d, J=5.5 Hz, 2H), 0.60 (br d, J=6.8 Hz, 2H) ppm.

Step 7: Preparation of tert-butyl (1-(3-((4-bromopyridin-2-yl)oxy)butyl)cyclopropyl)carbamate

To a solution of tert-butyl (1-(3-hydroxybutyl)cyclopropyl)carbamate (300 mg, 1.31 mmol, 1 eq) and 4-bromo-2-fluoro-pyridine (230.23 mg, 1.31 mmol, 1 eq) in DMF (10 mL) was added t-BuOK (220.20 mg, 1.96 mmol, 1.5 eq), and the mixture was stirred at 0° C. for 1 hour under N₂ atmosphere. The mixture was then concentrated and the resulting crude was purified by prep-HPLC (column: YMC Triart C18 150*25 mm*5 um; mobile phase: [water(FA)-ACN]; gradient:60%-90% B over 10 min) to give tert-butyl (1-(3-((4-bromopyridin-2-yl)oxy)butyl)cyclopropyl)carbamate (400 mg, 79.4% yield) as a yellow oil. LCMS: 385.1, 387.1 [M+H]⁺.

Step 8: Preparation of tert-butyl (1-(3-((4-((1-(tert-butyl)-3-((1S,3R)-3-((tert-butyldimethylsilyl)oxy)cyclopentyl)-1H-pyrazol-5-yl)amino)pyridin-2-yl)oxy)butyl)cyclopropyl)carbamate

The mixture of tert-butyl (1-(3-((4-bromopyridin-2-yl)oxy)butyl)cyclopropyl)carbamate (280 mg, 0.73 mmol, 1 eq), 1-(tert-butyl)-3-((1S,3R)-3-((tert-butyldimethylsilyl)oxy)cyclopentyl)-1H-pyrazol-5-amine (245.32 mg, 0.73 mmol, 1 eq), Pd₂(dba)₃ (66.55 mg, 0.073 mmol, 0.1 eq), Xantphos (84.10 mg, 0.15 mmol, 0.2 eq) and Cs₂CO₃ (710.33 mg, 2.18 mmol, 3 eq) in dioxane (5 mL) was heated and stirred at 90° C. for 1 hour under N₂ atmosphere. The reaction mixture was then cooled to room temperature, filtered through a short pad of celite and rinsed with dioxane (10 mL). The combined filtrate was concentrated and the resulting residue was purified by prep-TLC (PE/EA=2/1) to give tert-butyl (1-(3-((4-((1-(tert-butyl)-3-((1S,3R)-3-((tert-butyldimethylsilyl)oxy)cyclopentyl)-1H-pyrazol-5-yl)amino)pyridin-2-yl)oxy)butyl)cyclopropyl)carbamate (250 mg, 48.2% yield) as a yellow solid. LCMS: 642.5 [M+H]⁺.

Step 9: Preparation of tert-butyl (2-((4-(1-((tert-butoxycarbonyl)amino)cyclopropyl)butan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (1-(3-((4-((1-(tert-butyl)-3-((1S,3R)-3-((tert-butyldimethylsilyl)oxy)cyclopentyl)-1H-pyrazol-5-yl)amino)pyridin-2-yl)oxy)butyl)cyclopropyl)carbamate (250 mg, 0.39 mmol, 1 eq) in DCM (10 mL) was added DMAP (47.58 mg, 0.39 mmol, 1 eq) and Boc₂O (169.99 mg, 0.78 mmol, 0.18 mL, 2 eq), and the mixture was stirred at 25° C. for 1 hour under N₂ atmosphere. The mixture was then concentrated, and the resulting residue was purified by prep-TLC (PE/EA=2/1) to give tert-butyl (2-((4-(1-((tert-butoxycarbonyl)amino)cyclopropyl)butan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate (300 mg, 0.36 mmol, 90.0% purity) as a yellow solid. LCMS: 742.5 [M+H]⁺.

Step 10: Preparation of tert-butyl (2-((4-(1-((tert-butoxycarbonyl)amino)cyclopropyl)butan-2-yl)oxy)pyridin-4-yl)(1-(tert-butyl)-3-((1S,3R)-3-hydroxycyclopentyl)-1H-pyrazol-5-yl)carbamate

The solution of tert-butyl (2-((4-(1-((tert-butoxycarbonyl)amino)cyclopropyl)butan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate (300 mg, 0.40 mmol, 1 eq) and TBAF (1 M, 0.81 mL, 2 eq) in THF (6 mL) was stirred at 60° C. for 2 hours under N₂ atmosphere. After the reaction was completed, the mixture was cooled to room temperature. The mixture was concentrated and the resulting residue was purified by prep-TLC (PE/EA=1/1). to give tert-butyl (2-((4-(1-((tert-butoxycarbonyl)amino)cyclopropyl)butan-2-yl)oxy)pyridin-4-yl)(1-(tert-butyl)-3-((1S,3R)-3-hydroxycyclopentyl)-1H-pyrazol-5-yl)carbamate (220 mg, 86.7% yield) as a yellow solid. LCMS: 628.4 [M+H]⁺.

Step 11: Preparation of tert-butyl (2-((4-(1-((tert-butoxycarbonyl)amino)cyclopropyl)butan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((4-(1-((tert-butoxycarbonyl)amino)cyclopropyl)butan-2-yl)oxy)pyridin-4-yl)(1-(tert-butyl)-3-((1S,3R)-3-hydroxycyclopentyl)-1H-pyrazol-5-yl)carbamate (220 mg, 0.35 mmol, 1 eq) in THF (3 mL) were added DMAP (21.41 mg, 0.17 mmol, 0.5 eq) and Pyridine (138.59 mg, 1.75 mmol, 0.14 mL, 5 eq), followed by a solution of (4-nitrophenyl) carbonochloridate (211.90 mg, 1.05 mmol, 3 eq) in DCM (3 mL). After the completion of the addition, the mixture was stirred at 20° C. for 12 hours under N₂ atmosphere. The mixture was concentrated and the resulting residue was purified by prep-TLC (PE/EA=2/1) to give tert-butyl (2-((4-(1-((tert-butoxycarbonyl)amino)cyclopropyl)butan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (250 mg, 89.9% yield) as a yellow solid. LCMS: 793.5 [M+H]⁺.

Step 12: (1R,3S)-3-(5-((2-((4-(1-aminocyclopropyl)butan-2-yl)oxy)pyridin-4-yl)amino)-1-(tert-butyl)-1H-pyrazol-3-yl)cyclopentyl (4-nitrophenyl) carbonate

The solution of tert-butyl (2-((4-(1-((tert-butoxycarbonyl)amino)cyclopropyl)butan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (250 mg, 0.32 mmol, 1 eq) and TFA (3.07 g, 26.92 mmol, 2 mL, 85.40 eq) in DCM (8 mL) was stirred at 20° C. for 2 hours under N₂ atmosphere. After the completion of the reaction, the volatiles are removed under reduced pressure to give crude (1R,3S)-3-(5-((2-((4-(1-aminocyclopropyl)butan-2-yl)oxy)pyridin-4-yl)amino)-1-(tert-butyl)-1H-pyrazol-3-yl)cyclopentyl (4-nitrophenyl) carbonate (200 mg) as a yellow oil, which was used in the next step without further purification. LCMS: 593.4 [M+H]⁺.

Step 13: (1'S,3′R,6′R,Z)-1′-(tert-butyl)-6′-methylspiro[cyclopropane-1,9′-5,12-dioxa-3,10-diaza-4(4,2)-pyridina-2(3,5)-pyrazola-1(1,3)-cyclopentanacyclododecaphan]-11′-one and (1'S,3′R,6'S,Z)-1′-(tert-butyl)-6′-methylspiro[cyclopropane-1,9′-5,12-dioxa-3,10-diaza-4(4,2)-pyridina-2(3,5)-pyrazola-1(1,3)-cyclopentanacyclododecaphan]-11′-one

To a solution of (1R,3S)-3-(5-((2-((4-(1-aminocyclopropyl)butan-2-yl)oxy)pyridin-4-yl)amino)-1-(tert-butyl)-1H-pyrazol-3-yl)cyclopentyl (4-nitrophenyl) carbonate (150 mg, 0.25 mmol, 1 eq) in DMSO (40 mL) was added TEA (128.05 mg, 1.27 mmol, 0.17 mL, 5 eq), and the mixture was stirred at 100° C. for 0.5 hour under N₂ atmosphere. The mixture was then cooled to room temperature and concentrated under a high vacuum. The resulting crude was purified by prep-TLC (PE/EA=0/1) to afford two isolated isomers of (1'S,3′R,6′R,Z)-1′-(tert-butyl)-6′-methylspiro[cyclopropane-1,9′-5,12-dioxa-3,10-diaza-4(4,2)-pyridina-2(3,5)-pyrazola-1(1,3)-cyclopentanacyclododecaphan]-11′-one (25 mg, 22.3% yield) as a yellow solid and (1'S,3′R,6'S,Z)-1′-(tert-butyl)-6′-methylspiro[cyclopropane-1,9′-5,12-dioxa-3,10-diaza-4(4,2)-pyridina-2(3,5)-pyrazola-1(1,3)-cyclopentanacyclododecaphan]-11′-one (30 mg, 0.066 mmol, 26.4% yield) as a yellow solid. LCMS: 454.5 [M+H]⁺.

Step 14: Preparation of (1'S,3′R,6′R,Z)-6′-methylspiro[cyclopropane-1,9′-5,12-dioxa-3,10-diaza-4(4,2)-pyridina-2(3,5)-pyrazola-1 (1,3)-cyclopentanacyclododecaphan]-11′-one (compound 109)

A solution of (1'S,3′R,6′R,Z)-1′-(tert-butyl)-6′-methylspiro[cyclopropane-1,9′-5,12-dioxa-3,10-diaza-4(4,2)-pyridina-2(3,5)-pyrazola-1(1,3)-cyclopentanacyclododecaphan]-11′-one (25 mg, 0.055 mmol, 1 eq) in formic acid (0.2 mL) was stirred at 100° C. for 0.5 hour under N₂ atmosphere. The mixture was cooled to room temperature and concentrated to give a crude, which was purified by reversed phase column (0.1% FA) to afford (1'S,3′R,6′R,Z)-6′-methylspiro[cyclopropane-1,9′-5,12-dioxa-3,10-diaza-4(4,2)-pyridina-2(3,5)-pyrazola-1(1,3)-cyclopentanacyclododecaphan]-11′-one (14.48 mg, 66.3% yield) as a white solid. LCMS: 398.2 [M+H]⁺; ¹H NMR (400 MHz, DMSO-d₆) δ 12.10 (br s, 1H), 8.99-8.81 (m, 1H), 8.30-8.16 (m, 1H), 7.75 (br d, J=5.1 Hz, 1H), 7.61-7.09 (m, 1H), 6.46-6.37 (m, 1H), 6.33-6.26 (m, 1H), 6.26-6.25 (m, 1H), 6.10-5.86 (m, 1H), 5.07-4.90 (m, 1H), 4.89-4.23 (m, 1H), 3.24 (br s, 1H), 2.36 (br s, 1H), 2.12-1.86 (m, 4H), 1.81-1.46 (m, 5H), 1.31-1.15 (m, 3H), 0.65-0.47 (m, 4H) ppm.

Step 14: Preparation of (1'S,3′R,6'S,Z)-6′-methylspiro[cyclopropane-1,9′-5,12-dioxa-3,10-diaza-4(4,2)-pyridina-2(3,5)-pyrazola-1(1,3)-cyclopentanacyclododecaphan]-11′-one (compound 110)

A solution of (1'S,3′R,6'S,Z)-1′-(tert-butyl)-6′-methylspiro[cyclopropane-1,9′-5,12-dioxa-3,10-diaza-4(4,2)-pyridina-2(3,5)-pyrazola-1(1,3)-cyclopentanacyclododecaphan]-11′-one (30.00 mg, 0.066 mmol, 1 eq) in formic acid (0.2 mL) was stirred at 100° C. for 0.5 hour under N₂ atmosphere. After the completion of the reaction, the mixture was cooled to room temperature and concentrated to give a crude, which was purified by reversed phase column (0.1% FA) to give (1'S,3′R,6'S,Z)-6′-methylspiro[cyclopropane-1,9′-5,12-dioxa-3,10-diaza-4(4,2)-pyridina-2(3,5)-pyrazola-1 (1,3)-cyclopentanacyclododecaphan]-11′-one (10.98 mg, 41.9% yield) as a white solid. LCMS: 398.1 [M+H]⁺, H NMR (400 MHz, DMSO-d₆) δ 12.15 (s, 1H), 9.02-8.83 (m, 1H), 8.22 (br d, J=2.0 Hz, 1H), 7.72 (d, J=5.5 Hz, 1H), 7.63-7.51 (m, 1H), 6.48-6.27 (m, 2H), 6.11-5.99 (m, 1H), 5.25-4.97 (m, 1H), 4.65-4.31 (m, 1H), 3.23-3.15 (m, 1H), 2.45-2.39 (m, 1H), 2.10-2.00 (m, 2H), 1.99-1.89 (m, 1H), 1.83-1.73 (m, 3H), 1.63 (br dd, J=4.6, 7.6 Hz, 2H), 1.20-1.11 (m, 3H), 1.06-0.92 (m, 1H), 0.66-0.53 (m, 3H), 0.51-0.40 (m, 1H) ppm.

Example 15: (1¹S,1³R,6R,Z)-6-methyl-2¹H-5,12-dioxa-3,10-diaza-4(4,2)-pyridina-2(3,5)-pyrazola-1(1,3)-cyclopentanacyclododecaphan-11-one (compound 11) and (1¹S,1³R,6S,Z)-6-methyl-2¹H-5,12-dioxa-3,10-diaza-4(4,2)-pyridina-2(3,5)-pyrazola-1(1,3)-cyclopentanacyclododecaphan-11-one (compound 12)

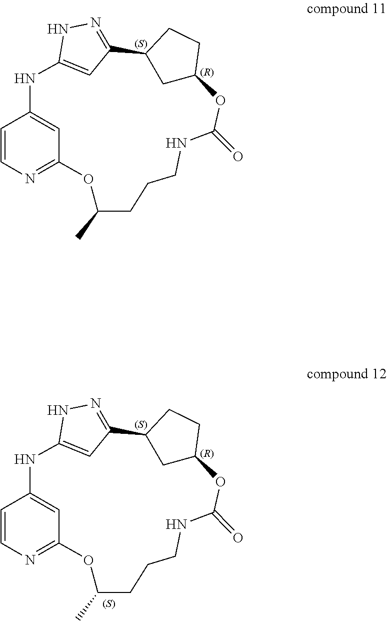

Step 1: Preparation of tert-butyl (4-(methoxy(methyl)amino)-4-oxobutyl)carbamate

A mixture of 4-((tert-butoxycarbonyl)amino)butanoic acid (5 g, 24.60 mmol, 1 eq), HATU (14.03 g, 36.90 mmol, 1.5 eq), DIEA (9.54 g, 73.81 mmol, 12.86 mL, 3 eq) and N,O-dimethylhydroxylamine hydrochloride (3.60 g, 36.90 mmol, 1.5 eq) in DMF (60 mL) was stirred at 25° C. for 12 hours. The reaction mixture was then diluted with H₂O (100 mL) and extracted with dichloromethane (100 mL×3). The combined organic layers were washed with brine (100 mL), dried over Na₂SO₄, filtered, and the filtrate was concentrated under reduced pressure. The resulting residue was purified by column chromatography to give tert-butyl (4-(methoxy(methyl)amino)-4-oxobutyl)carbamate (6 g, 99.0% yield). ¹HNMR (400 MHz, DMSO-d₆) δ 6.84-6.82 (br t, J=5.3 Hz, 1H), 3.64 (s, 3H), 3.07 (s, 3H), 2.95-2.92 (m, 2H), 2.69 (s, 9H), 2.37-2.34 (m, 2H), 1.62-1.55 (m, 2H), 1.37 (s, 9H) ppm.

Step 2: Preparation of tert-butyl (4-oxopentyl)carbamate

To a solution of tert-butyl (4-(methoxy(methyl)amino)-4-oxobutyl)carbamate (1 g, 4.06 mmol, 1 eq) in THF (20 mL) was slowly added MeMgBr (3 M, 4.06 mL, 3 eq) at 0° C. under N₂ atmosphere. After the completion of the addition, the mixture was stirred at 0° C. for 1 hour under N₂ atmosphere. The reaction mixture was then quenched with water (30 ml) and extracted with Ethyl acetate (100 mL×2). The combined organic layers were washed with brine (100 mL), dried over Na₂SO₄, filtered, and the filtrate was concentrated under reduced pressure. The resulting residue was purified by column chromatography to give tert-butyl (4-oxopentyl)carbamate (465 mg, 56.9% yield). ¹HNMR (400 MHz, DMSO-d₆) δ 6.79 (m, 1H), 2.89-2.84 (m, 2H), 2.83-2.42 (t, J=7.2 Hz, 2H), 2.06 (s, 3H), 1.57-1.50 (m, 2H), 1.37 (s, 9H) ppm.

Step 3: Preparation of tert-butyl (4-hydroxypentyl)carbamate

To a solution of tert-butyl (4-oxopentyl)carbamate (465 mg, 2.31 mmol, 1 eq) in EtOH (10 mL) was added NaBH₄ (174.82 mg, 4.62 mmol, 2 eq) in portions at 0° C. under N₂ atmosphere. After the completion of the addition, the mixture was stirred at 30° C. for 3 hours under N₂ atmosphere. Then, the reaction mixture was quenched with saturated aqueous NH₄Cl (20 mL) and extracted with ethyl acetate (20 mL×2). The combined organic layers were washed with brine (20 mL×2), dried over Na₂SO₄, filtered, and the filtrate was concentrated under reduced pressure. The resulting residue was purified by column chromatography to give tert-butyl (4-hydroxypentyl)carbamate (530 mg, 99.7% yield). ¹HNMR (400 MHz, DMSO-d₆) δ 6.75 (br d, J=4.6 Hz, 1H), 4.35-4.31 (m, 1H), 3.58-3.52 (m, 1H), 2.90-2.85 (m, 2H), 1.37 (s, 11H), 1.31-1.14 (m, 2H), 1.11-0.99 (m, 3H) ppm.

Step 4: Preparation of tert-butyl (4-((4-bromopyridin-2-yl)oxy)pentyl)carbamate

To a solution of tert-butyl (4-hydroxypentyl)carbamate (470 mg, 2.31 mmol, 1 eq) and 4-bromo-2-fluoro-pyridine (406.90 mg, 2.31 mmol, 1 eq) in THF (10 mL) was added t-BuOK (389.17 mg, 3.47 mmol, 1.5 eq) at 0° C., and the mixture was warmed to 30° C. and stirred for 12 hours under N₂ atmosphere. After the starting materials were consumed, the reaction mixture was filtered through a pad of celite and rinsed with THF (10 mL). The combined filtrate was concentrated under reduced pressure to give a residue, which was purified by prep-TLC. The desired tert-butyl (4-((4-bromopyridin-2-yl)oxy)pentyl)carbamate (640 mg, 74.0% yield) was obtained. LCMS: 359.1, 361.1 [M+H]⁺.

Step 5: Preparation of tert-butyl (4-((4-((1-(tert-butyl)-3-((1S,3R)-3-((tert-butyldimethylsilyl)oxy)cyclopentyl)-1H-pyrazol-5-yl)amino)pyridin-2-yl)oxy)pentyl)carbamate

The mixture of tert-butyl (4-((4-bromopyridin-2-yl)oxy)pentyl)carbamate (390 mg, 1.09 mmol, 1 eq), 1-(tert-butyl)-3-((1S,3R)-3-((tert-butyldimethylsilyl)oxy)cyclopentyl)-1H-pyrazol-5-amine (366.46 mg, 1.09 mmol, 1 eq), Xantphos (103.50 mg, 0.21 mmol, 0.2 eq), Cs₂CO₃ (1.06 g, 3.26 mmol, 3 eq) and Pd₂(dba)₃ (99.41 mg, 0.11 mmol, 0.1 eq) in dioxane (5 mL) was stirred at 90° C. for 12 hours under N₂ atmosphere. After the reaction was completed, the mixture was cooled to room temperature and filtered through a pad of celite, rinsed with ethyl acetate (30 mL). The combined filtrate was concentrated under reduced pressure. The resulting residue was purified by prep-TLC (Petroleum ether/Ethyl acetate=5/1) to give tert-butyl (4-((4-((1-(tert-butyl)-3-((1S,3R)-3-((tert-butyldimethylsilyl)oxy)cyclopentyl)-1H-pyrazol-5-yl)amino)pyridin-2-yl)oxy)pentyl)carbamate (450 mg, 67.3% yield). LCMS: 616.6 [M+H]⁺.

Step 6: Preparation of tert-butyl (2-((5-((tert-butoxycarbonyl)amino)pentan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (4-((4-((1-(tert-butyl)-3-((1S,3R)-3-((tert-butyldimethylsilyl)oxy)cyclopentyl)-1H-pyrazol-5-yl)amino)pyridin-2-yl)oxy)pentyl)carbamate (450 mg, 0.73 mmol, 1 eq) in THF (6 mL) was added Boc₂O (478.36 mg, 2.19 mmol, 3 eq), TEA (332.69 mg, 3.29 mmol, 457.61 μL, 4.5 eq) and DMAP (133.89 mg, 1.10 mmol, 1.5 eq) at 0° C., and the mixture was then warm to 50° C. and stirred for 12 hours under N₂ atmosphere. After the reaction was completed, the mixture was cooled to room temperature, and concentrated under reduced pressure. The resulting residue was purified by column chromatography to give tert-butyl (2-((5-((tert-butoxycarbonyl)amino)pentan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate (220 mg, 42.1% yield). LCMS: 716.5 [M+H]⁺.

Step 7: Preparation of tert-butyl (2-((5-((tert-butoxycarbonyl)amino)pentan-2-yl)oxy)pyridin-4-yl)(1-(tert-butyl)-3-((1S,3R)-3-hydroxycyclopentyl)-1H-pyrazol-5-yl)carbamate

To a solution of tert-butyl (2-((5-((tert-butoxycarbonyl)amino)pentan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate (220 mg, 0.31 mmol, 1 eq) in THF (4 mL) was added TBAF (1 M in THF, 0.8 mL, 2.6 eq), and the mixture was then stirred at 60° C. for 2 hours under N₂ atmosphere. After the completion of the reaction, the mixture was cooled to room temperature, and concentrated under reduced pressure. The resulting residue was purified by prep-TLC (Petroleum ether/Ethyl acetate=3/1) to give tert-butyl (2-((5-((tert-butoxycarbonyl)amino)pentan-2-yl)oxy)pyridin-4-yl)(1-(tert-butyl)-3-((1S,3R)-3-hydroxycyclopentyl)-1H-pyrazol-5-yl)carbamate (145 mg, 78.4% yield). LCMS: 602.4 [M+H]⁺.

Step 8: Preparation of tert-butyl (2-((5-((tert-butoxycarbonyl)amino)pentan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((5-((tert-butoxycarbonyl)amino)pentan-2-yl)oxy)pyridin-4-yl)(1-(tert-butyl)-3-((1S,3R)-3-hydroxycyclopentyl)-1H-pyrazol-5-yl)carbamate (145 mg, 0.24 mmol, 1 eq) in DCM (2 mL) was added Pyridine (114.36 mg, 1.45 mmol, 0.12 mL, 6 eq) and DMAP (14.72 mg, 0.12 mmol, 0.5 eq), followed by the addition of a solution of (4-nitrophenyl) carbonochloridate (242.84 mg, 1.20 mmol, 5 eq) in DCM (2 mL) at 0° C. under N₂ atmosphere. After the completion of the addition, the mixture was warmed to 30° C. and stirred for 12 hours under N₂ atmosphere. The mixture was then cooled to room temperature, diluted with H₂O (20 mL) and extracted with dichloromethane (20 mL×3). The combined organic layers were washed with brine (20 mL), dried over Na₂SO₄, filtered, and the filtrate was concentrated under reduced pressure. The resulting residue was purified by prep-TLC (Petroleum ether/Ethyl acetate=6/1) to give tert-butyl (2-((5-((tert-butoxycarbonyl)amino)pentan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (150 mg, 81.2% yield). LCMS: 767.4 [M+H]⁺.

Step 9: Preparation of (1R,3S)-3-(5-((2-((5-aminopentan-2-yl)oxy)pyridin-4-yl)amino)-1-(tert-butyl)-1H-pyrazol-3-yl)cyclopentyl (4-nitrophenyl) carbonate

To a solution of tert-butyl (2-((5-((tert-butoxycarbonyl)amino)pentan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (150 mg, 0.19 mmol, 1 eq) in DCM (3 mL) was added TFA (914.90 mg, 8.02 mmol, 0.60 mL, 41.02 eq), and the mixture was stirred at 30° C. for 12 hours under N₂ atmosphere. The reaction mixture was then concentrated under reduced pressure to give the desired crude (1R,3S)-3-(5-((2-((5-aminopentan-2-yl)oxy)pyridin-4-yl)amino)-1-(tert-butyl)-1H-pyrazol-3-yl)cyclopentyl (4-nitrophenyl) carbonate (100 mg, 90.2% yield). LCMS: 567.4 [M+H]⁺.

Step 10: Preparation of (1¹S,1³R,Z)-2¹-(tert-butyl)-6-methyl-2¹H-5,12-dioxa-3,10-diaza-4(4,2)-pyridina-2(3,5)-pyrazola-1(1,3)-cyclopentanacyclododecaphan-11-one

To a solution of (1R,3S)-3-(5-((2-((5-aminopentan-2-yl)oxy)pyridin-4-yl)amino)-1-(tert-butyl)-1H-pyrazol-3-yl)cyclopentyl (4-nitrophenyl) carbonate (100 mg, 0.18 mmol, 1 eq) in THF (20 mL) was added TEA (357.15 mg, 3.53 mmol, 0.49 mL, 20 eq), and the mixture was stirred at 20° C. for 1 hour under N₂ atmosphere. After the completion of the reaction, the reaction mixture was concentrated under reduced pressure. The resulting residue was purified by prep-TLC (Petroleum ether/Ethyl acetate=1/1) to give (1¹S,1³R,Z)-2¹-(tert-butyl)-6-methyl-2¹H-5,12-dioxa-3,10-diaza-4(4,2)-pyridina-2(3,5)-pyrazola-1(1,3)-cyclopentanacyclododecaphan-11-one (55 mg, 72.9% yield). LCMS: 428.4 [M+H]⁺.

Step 11: (1¹S,1³R,6R,Z)-6-methyl-2¹H-5,12-dioxa-3,10-diaza-4(4,2)-pyridina-2(3,5)-pyrazola-1(1,3)-cyclopentanacyclododecaphan-11-one (compound 11) and (1¹S,1³R,6S,Z)-6-methyl-2¹H-5,12-dioxa-3,10-diaza-4(4,2)-pyridina-2(3,5)-pyrazola-1(1,3)-cyclopentanacyclododecaphan-11-one (compound 12

The solution of (1¹S,1³R,Z)-2¹-(tert-butyl)-6-methyl-2¹H-5,12-dioxa-3,10-diaza-4(4,2)-pyridina-2(3,5)-pyrazola-1(1,3)-cyclopentanacyclododecaphan-11-one (55 mg, 0.128 mmol, 1 eq) in HCOOH (2 mL) was stirred at 100° C. for 0.5 hour under N₂ atmosphere. After the reaction was completed, the mixture was cooled to room temperature and was adjusted to pH=10 with saturated NH₃H₂O. The volatiles was then removed under reduced pressure. The resulting residue was purified by prep-TLC (Dichloromethane: Methanol=10:1), and further separated by SFC (column: DAICEL CHIRALCEL OD (250 mm*30 mm, 10 um); mobile phase: [CO₂-MeOH(0.1% NH₃H₂O)]; B %:50%, isocratic elution mode) to afford (1¹S,1³R,6R,Z)-6-methyl-2¹H-5,12-dioxa-3,10-diaza-4(4,2)-pyridina-2(3,5)-pyrazola-1 (1,3)-cyclopentanacyclododecaphan-11-one (11.28 mg, 22.9% yield) and (1¹S,1³R,6S,Z)-6-methyl-2¹H-5,12-dioxa-3,10-diaza-4(4,2)-pyridina-2(3,5)-pyrazola-1 (1,3)-cyclopentanacyclododecaphan-11-one (8.45 mg, 17.0% yield).

(1¹S,1³R,6R,Z)-6-methyl-2¹H-5,12-dioxa-3,10-diaza-4(4,2)-pyridina-2(3,5)-pyrazola-1(1,3)-cyclopentanacyclododecaphan-11-one LCMS: 372.3 [M+H]⁺; ¹H NMR (400 MHz, DMSO-d₆) 5=12.15-12.01 (m, 1H), 8.93-8.82 (m, 1H), 7.72 (br d, J=5.0 Hz, 1H), 6.85-6.65 (m, 1H), 6.43-6.38 (m, 1H), 6.33-6.24 (m, 1H), 6.02-5.92 (m, 1H), 5.16-4.94 (m, 1H), 3.01-2.75 (m, 2H), 2.09-1.93 (m, 2H), 1.83-1.73 (m, 4H), 1.56-1.42 (m, 3H), 1.28-1.19 (m, 6H) ppm.

(1¹S,1³R,6S,Z)-6-methyl-2¹H-5,12-dioxa-3,10-diaza-4(4,2)-pyridina-2(3,5)-pyrazola-1(1,3)-cyclopentanacyclododecaphan-11-one LCMS: 372.3 [M+H]⁺; ¹H NMR (400 MHz, DMSO-d₆) δ 12.19-12.00 (m, 1H), 8.99-8.78 (m, 1H), 7.80-7.68 (m, 1H), 6.95-6.71 (m, 1H), 6.47-6.36 (m, 1H), 6.32-6.21 (m, 1H), 6.02-5.94 (m, 1H), 4.95-4.84 (m, 1H), 3.23-3.15 (m, 2H), 2.09-1.93 (m, 2H), 1.86-1.71 (m, 4H), 1.53-1.44 (m, 3H), 1.24 (br d, J=8.0 Hz, 6H) ppm.

Example 16: Synthesis of (1¹S,1³R,6R,Z)-4⁶-(difluoromethyl)-6-methyl-2¹H-5,11-dioxa-3,9-diaza-4(4,2)-pyridina-2(3,5)-pyrazola-8(1,3)-bicyclo[1 0.1.1]pentana-1(1 3)-cyclopentanacycloundecaphan-10-one (compound 24) and (1¹S,1³R,6S,Z)-4⁶-(difluoromethyl)-6-m ethyl-2¹H-5,11-dioxa-3,9-diaza-4(4,2)-pyridina-2(3,5)-pyrazola-8(1,3)-bicyclo[1.1 0.1]pentana-1(1,3)-cyclopentanacycloundecaphan-10-one (compound 25)

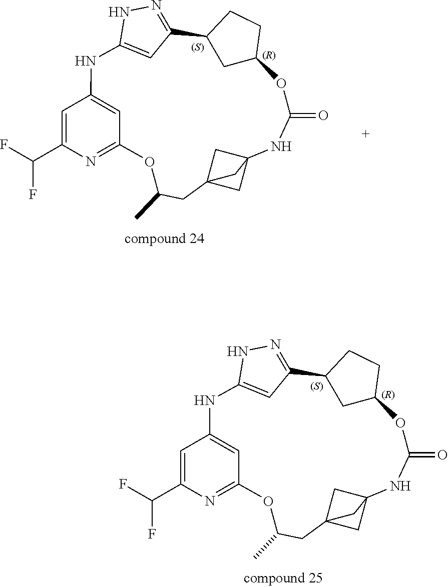

Step 1: Preparation of tert-butyl (3-(2-hydroxypropyl)bicyclo[1.1.1]pentan-1-yl)carbamate

To a solution of tert-butyl (3-(2-oxoethyl)bicyclo[1.1 0.1]pentan-1-yl)carbamate (1.27 g, 5.64 mmol, 1 eq) in THF (15 mL) was added MeMgBr (3 M, 11.27 mL, 6 eq) at −78° C., and the mixture was stirred at −78° C. for 6 hours under N₂ atmosphere. After the aldehyde was consumed, the reaction mixture was quenched with saturated aqueous NH₄Cl (30 mL) at 0° C. and extracted with ethyl acetate (30 mL×2). The combined organic layers were washed with brine (30 mL), dried over Na₂SO₄, filtered, and the filtrate was concentrated under reduced pressure. The resulting residue was purified by column chromatography to afford tert-butyl (3-(2-hydroxypropyl)bicyclo[1.1.1]pentan-1-yl)carbamate (900 mg, 66.2% yield) as a white solid. ¹H NMR (400 MHz, DMSO-d₆) δ 4.93 (s, 1H), 3.86-3.81 (m, 1H), 2.65 (s, 1H), 1.94-1.93 (m, 6H), 1.70-1.69 (m, 2H), 1.44 (s, 9H), 1.25-1.19 (d, 3H) ppm.

Step 2: Preparation of tert-butyl (3-(2-((4-bromo-6-(difluoromethyl)pyridin-2-yl)oxy)propyl)bicyclo[1.1.1]pentan-1-yl)carbamate

To a solution of tert-butyl (3-(2-hydroxypropyl)bicyclo[1.1.1]pentan-1-yl)carbamate (400 mg, 1.66 mmol, 1 eq) in DMF (20 mL) was added 4-bromo-2-chloro-6-(difluoromethyl)pyridine (602.79 mg, 2.49 mmol, 1.5 eq) and t-BuOK (371.98 mg, 3.32 mmol, 2 eq), and the mixture was stirred at 20° C. for 8 hours under N₂ atmosphere. The reaction mixture was then quenched with water (50 mL) and extracted with ethyl acetate (30 mL×2). The combined organic layers were washed with brine (50 mL), dried over Na₂SO₄, filtered, and the filtrate was concentrated under reduced pressure. The resulting residue was purified by column chromatography to afford tert-butyl (3-(2-((4-bromo-6-(difluoromethyl)pyridin-2-yl)oxy)propyl)bicyclo[1.1.1]pentan-1-yl)carbamate (350 mg, 47.2% yield) as a yellow oil. LCMS: 447.1 [M+H]⁺, 347.2 [M−Boc+H]⁺.

Step 3: Preparation of tert-butyl (3-(2-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)oxy)propyl)bicyclo[1.1.1]pentan-1-yl)carbamate

A mixture of tert-butyl (3-(2-((4-bromo-6-(difluoromethyl)pyridin-2-yl)oxy)propyl)bicyclo[1.1.1]pentan-1-yl)carbamate (320 mg, 0.715 mmol, 1 eq), 1-(tert-butyl)-3-((1S,3R)-3-((tert-butyldimethylsilyl)oxy)cyclopentyl)-1H-pyrazol-5-amine (289.79 mg, 0.859 mmol, 1.2 eq), Pd₂(dba)₃ (65.51 mg, 0.072 mmol, 0.1 eq), Xantphos (82.79 mg, 0.143 mmol, 0.2 eq) and Cs₂CO₃ (699.26 mg, 2.15 mmol, 3 eq) in dioxane (20 mL) was stirred at 90° C. for 12 hours under N₂ atmosphere. After the completion of the reaction, the mixture was cooled to room temperature, poured into water (50 mL) and extracted with EtOAc (30 mL×3). The organic layers were washed with brine (30 mL), dried over Na₂SO₄, filtered and the filtrate was evaporated under reduced pressure. The resulting residue was purified by column chromatography to afford tert-butyl (3-(2-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)oxy)propyl)bicyclo[1.1.1]pentan-1-yl)carbamate (360 mg, 53.6% yield) as a yellow oil. LCMS: 704.5 [M+H]⁺.

Step 4: Preparation of tert-butyl (2-((1-(3-((tert-butoxycarbonyl)amino)bicyclo[1.1.1]pentan-1-yl)propan-2-yl)oxy)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (3-(2-((4-((1-(tert-butyl)-3-((1S,3R)-3-((tert-butyldimethylsilyl)oxy)cyclopentyl)-1H-pyrazol-5-yl)amino)-6-(difluoromethyl)pyridin-2-yl)oxy)propyl)bicyclo[1.1.1]pentan-1-yl)carbamate (360 mg, 0.511 mmol, 1 eq) in DCM (20 mL) was added Boc₂O (167.41 mg, 0.767 mmol, 1.5 eq) and DMAP (62.47 mg, 0.511 mmol, 1 eq), and the mixture was stirred at 20° C. for 3 hours. After the completion of the reaction, the reaction mixture was concentrated under reduced pressure. The resulting residue was purified by column chromatography to afford tert-butyl (2-((1-(3-((tert-butoxycarbonyl)amino)bicyclo[1.1.1]pentan-1-yl)propan-2-yl)oxy)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate (255 mg, 62.0% yield) as a yellow oil. LCMS: 804.5 [M+H]⁺.

Step 5: Preparation of tert-butyl (2-((1-(3-((tert-butoxycarbonyl)amino)bicyclo[1.1.1]pentan-1-yl)propan-2-yl)oxy)-6-(difluoromethyl)pyridin-4-yl)(1-(tert-butyl)-3-((1S,3R)-3-hydroxycyclopentyl)-1H-pyrazol-5-yl)carbamate

The solution of tert-butyl (2-((1-(3-((tert-butoxycarbonyl)amino)bicyclo[1.1.1]pentan-1-yl)propan-2-yl)oxy)-6-(difluoromethyl)pyridin-4-yl)(1-(tert-butyl)-3-((1S,3R)-3-((tert-butyldimethylsilyl)oxy)cyclopentyl)-1H-pyrazol-5-yl)carbamate (255 mg, 0.317 mmol, 1 eq) and TBAF (414.59 mg, 1.59 mmol, 5 eq) in THF (10 mL) was stirred at 20° C. for 8 hours. The reaction mixture was then concentrated, diluted with water (20 mL) and extracted with ethyl acetate (20 mL×2). The combined organic layers were washed with brine (20 mL), dried over Na₂SO₄, filtered, and the filtrate was concentrated to afford tert-butyl (2-((1-(3-((tert-butoxycarbonyl)amino)bicyclo[1.1.1]pentan-1-yl)propan-2-yl)oxy)-6-(difluoromethyl)pyridin-4-yl)(1-(tert-butyl)-3-((1S,3R)-3-hydroxycyclopentyl)-1H-pyrazol-5-yl)carbamate (210 mg, 86.4% yield) as a yellow solid. LCMS: 690.3 [M+H]⁺.

Step 6: Preparation of (1R,3S)-3-(5-((2-((1-(3-aminobicyclo[1.1.1]pentan-1-yl)propan-2-yl)oxy)-6-(difluoromethyl)pyridin-4-yl)amino)-1-(tert-butyl)-1H-pyrazol-3-yl)cyclopentyl (4-nitrophenyl) carbonate

To a solution of tert-butyl (2-((1-(3-((tert-butoxycarbonyl)amino)bicyclo[1.1.1]pentan-1-yl)propan-2-yl)oxy)-6-(difluoromethyl)pyridin-4-yl)(1-(tert-butyl)-3-((1S,3R)-3-hydroxycyclopentyl)-1H-pyrazol-5-yl)carbamate (210 mg, 0.304 mmol, 1 eq) in DCM (10 mL) was added Pyridine (144.48 mg, 1.83 mmol, 6 eq) and DMAP (37.19 mg, 0.304 mmol, 1 eq), followed by the addition of a solution of (4-nitrophenyl) carbonochloridate (184.08 mg, 0.913 mmol, 3 eq) in DCM (10 mL), and the mixture was stirred at 45° C. for 16 hours under N₂ atmosphere. After the completion of the reaction, the mixture was cooled to room temperature and concentrated. The residue was treated with water (20 mL) and ethyl acetate (30 mL). The organic layer was collected, and the aqueous phase was extracted with ethyl acetate (30 mL×2). The combined organic layers were washed with brine (20 mL), dried over Na₂SO₄, filtered, and the filtrate was concentrated under reduced pressure. The resulting residue was purified by column chromatography to afford (1R,3S)-3-(5-((2-((1-(3-aminobicyclo[1.1.1]pentan-1-yl)propan-2-yl)oxy)-6-(difluoromethyl)pyridin-4-yl)amino)-1-(tert-butyl)-1H-pyrazol-3-yl)cyclopentyl (4-nitrophenyl) carbonate (175 mg, 0.205 mmol, 67.2% yield) as a yellow oil. LCMS: 855.3 [M+H]⁺.

Step 7: Preparation of (1R,3S)-3-(5-((2-((1-(3-aminobicyclo[1.1.1]pentan-1-yl)propan-2-yl)oxy)-6-(difluoromethyl)pyridin-4-yl)amino)-1-(tert-butyl)-1H-pyrazol-3-yl)cyclopentyl (4-nitrophenyl) carbonate

The solution of (1R,3S)-3-(5-((2-((1-(3-aminobicyclo[1.1.1]pentan-1-yl)propan-2-yl)oxy)-6-(difluoromethyl)pyridin-4-yl)amino)-1-(tert-butyl)-1H-pyrazol-3-yl)cyclopentyl (4-nitrophenyl) carbonate (175 mg, 0.205 mmol, 1 eq) and TFA (116.70 mg, 1.02 mmol, 5 eq) in DCM (3 mL) was stirred at 40° C. for 0.5 hour. After the completion of the reaction, the mixture was cooled to room temperature. The reaction mixture was concentrated in vacuo to afford (1R,3S)-3-(5-((2-((1-(3-aminobicyclo[1.1.1]pentan-1-yl)propan-2-yl)oxy)-6-(difluoromethyl)pyridin-4-yl)amino)-1-(tert-butyl)-1H-pyrazol-3-yl)cyclopentyl (4-nitrophenyl) carbonate (120 mg, 89.5% yield) as a yellow solid. LCMS: 655.3 [M+H]⁺.

Step 8: Preparation of (1¹S,1³R,Z)-2¹-(tert-butyl)-4⁶-(difluoromethyl)-6-methyl-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one

To a solution of (1R,3S)-3-(5-((2-((1-(3-aminobicyclo[1.1.1]pentan-1-yl)propan-2-yl)oxy)-6-(difluoromethyl)pyridin-4-yl)amino)-1-(tert-butyl)-1H-pyrazol-3-yl)cyclopentyl (4-nitrophenyl) carbonate (120 mg, 0.183 mmol, 1 eq) in THF (20 mL) was added DIEA (118.44 mg, 0.916 mmol, 0.16 mL, 5 eq), and the mixture was stirred at 80° C. for 2 hours under N₂ atmosphere. After the completion of the reaction, the mixture was cooled to room temperature and concentrated under reduced pressure. The resulting residue was purified by prep-TLC (Petroleum ether/Ethyl acetate=2:1) to afford (1¹S,1³R,Z)-2¹-(tert-butyl)-4⁶-(difluoromethyl)-6-methyl-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one (80 mg, 84.7% yield) as a yellow solid. LCMS: 516.3 [M+H]⁺.

Step 9: Preparation of (1¹S,1³R,6R,Z)-4⁶-(difluoromethyl)-6-methyl-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one and (1¹S,1³R,6S,Z)-4⁶-(difluoromethyl)-6-methyl-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one

The mixture of (1¹S,1³R,Z)-2¹-(tert-butyl)-4⁶-(difluoromethyl)-6-methyl-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one (80 mg, 0.155 mmol, 1 eq) in formic acid (2 mL) was heated and stirred at 100° C. for 1 hour under N₂ atmosphere. After the completion of the reaction, the mixture was cooled to room temperature and concentrated under reduced pressure. The resulting residue was purified by SFC (column: DAICEL CHIRALPAK IG (250 mm*30 mm, 10 um); mobile phase: [CO₂-EtOH (0.1% NH₃·H₂O)]; B %:55%, isocratic elution mode) to afford (1¹S,1³R,6R,Z)-4⁶-(difluoromethyl)-6-methyl-2¹H-5,11-dioxa-3,9-diaza-4(4,2)-pyridina-2(3,5)-pyrazola-8(1,3)-bicyclo[1 0.1.1]pentana-1(1,3)-cyclopentanacycloundecaphan-10-one (26.28 mg, 36.9% yield) as a white solid and (1¹S,1³R,6S,Z)-4⁶-(difluoromethyl)-6-methyl-2¹H-5,11-dioxa-3,9-diaza-4(4,2)-pyridina-2(3,5)-pyrazola-8(1,3)-bicyclo[1.1 0.1]pentana-1(1,3)-cyclopentanacycloundecaphan-10-one (29.37 mg, 41.2% yield) as a white solid.

(1¹S,1³R,6R,Z)-4⁶-(difluoromethyl)-6-methyl-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one LCMS: 460.2 [M+H]⁺, 482.2 [M+Na]+; ¹H NMR (400 MHz, DMSO-d₆) 5=11.98 (s, 1H), 8.69 (s, 1H), 7.44 (s, 1H), 6.79-6.51 (t, 1H), 6.41 (s, 1H), 6.22 (s, 1H), 5.87 (s, 1H), 5.19-5.18 (m, 1H), 4.23-4.20 (m, 1H), 3.32-3.20 (m, 1H), 2.51-2.33 (m, 1H), 2.08-1.95 (m, 1H), 1.82-1.81 (m, 2H), 1.79-1.72 (m, 6H), 1.65-1.62 (m, 4H), 1.29-1.28 (d, J=5.6, 3H) ppm.

(1¹S,1³R,6S,Z)-4⁶-(difluoromethyl)-6-methyl-2¹H-5,11-dioxa-3,9-diaza-4(4,2)-pyridina-2(3,5)-pyrazola-8(1,3)-bicyclo[1.1.1]pentana-1(1,3)-cyclopentanacycloundecaphan-10-one LCMS: 460.2 [M+H]⁺, 482.2 [M+Na]+; ¹H NMR (400 MHz, DMSO-d₆) 5=8.55 (s, 1H), 7.43 (s, 1H), 6.78-6.50 (m, 1H), 6.39 (s, 1H), 6.16 (s, 1H), 5.97 (s, 1H), 5.11-5.09 (m, 1H), 4.25-4.22 (m, 1H), 3.40-3.24 (m, 1H), 2.50-2.44 (m, 1H), 1.90-1.88 (m, 5H), 1.86-1.84 (m, 4H), 1.75-1.73 (m, 3H), 1.65-1.63 (m, 1H), 1.27-1.24 (d, J=5.6, 3H) ppm.

Example 17: Synthesis of (1¹S,1³R,5R,Z)-5-methyl-2¹H-6,12-dioxa-3,10-diaza-4(4,2)-pyridina-2(3,5)-pyrazola-1(1,3)-cyclopentanacyclododecaphan-11-one (compound 71) and (1¹S,1³R,5S,Z)-5-methyl-2¹H-6,12-dioxa-3,10-diaza-4(4,2)-pyridina-2(3,5)-pyrazola-1(1,3)-cyclopentanacyclododecaphan-11-one (compound 72)

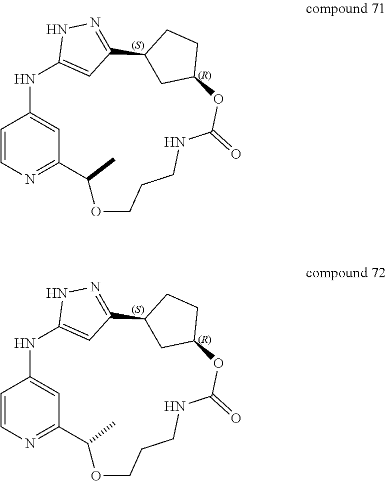

Step 1: Preparation of tert-butyl (3-(1-(4-bromopyridin-2-yl)ethoxy)propyl)carbamate

To a solution of 1-(4-bromopyridin-2-yl)ethan-1-ol (800 mg, 3.96 mmol, 1 eq) in DMF (3 mL) was added NaH (316.73 mg, 7.92 mmol, 60% purity, 2 eq), and the mixture was stirred at 0° C. for 2.5 hours under nitrogen atmosphere. Then, tert-butyl (3-bromopropyl)carbamate (1.41 g, 5.94 mmol, 1.5 eq) was added at 0° C. and the mixture was kept stirring at 0° C. for 2.5 hours under nitrogen atmosphere. After the completion of the reaction, the reaction mixture was quenched with saturated aqueous NH₄Cl (50 mL) and extracted with ethyl acetate (40 mL×2). The combined organic layers were washed with brine (50 mL), dried over Na₂SO₄, filtered and the filtrate was concentrated under reduced pressure. The resulting residue was purified by column chromatography to afford tert-butyl (3-(1-(4-bromopyridin-2-yl)ethoxy)propyl)carbamate (585 mg, 41.1% yield) as a white solid. LCMS: 359.1, 361.1 [M+H]+

Step 2: Preparation of tert-butyl (3-(1-(4-((1-(tert-butyl)-3-((1S,3R)-3-((tert-butyldimethylsilyl)oxy)cyclopentyl)-1H-pyrazol-5-yl)amino)pyridin-2-yl)ethoxy)propyl)carbamate

A mixture of tert-butyl (3-(1-(4-bromopyridin-2-yl)ethoxy)propyl)carbamate (620 mg, 1.73 mmol, 1 eq), 1-(tert-butyl)-3-((1S,3R)-3-((tert-butyldimethylsilyl)oxy)cyclopentyl)-1H-pyrazol-5-amine (640.84 mg, 1.90 mmol, 1.1 eq), Pd₂(dba)₃ (158.03 mg, 0.172 mmol, 0.1 eq), Xantphos (199.71 mg, 0.345 mmol, 0.2 eq) and Cs₂CO₃ (1.69 g, 5.18 mmol, 3 eq) in dioxane (20 mL) was stirred at 90° C. for 12 hours under N₂ atmosphere. After the completion of the reaction, the mixture was cooled to room temperature. The reaction mixture was diluted with water (50 mL) and extracted with EtOAc (50 mL×2). The combined organic layers were washed with brine (50 mL), dried over Na₂SO₄, filtered, and the filtrate was concentrated under reduced pressure. The resulting residue was purified by column chromatography to afford tert-butyl (3-(1-(4-((1-(tert-butyl)-3-((1S,3R)-3-((tert-butyldimethylsilyl)oxy)cyclopentyl)-1H-pyrazol-5-yl)amino)pyridin-2-yl)ethoxy)propyl)carbamate (1.1 g, 93.1% yield) as a yellow oil. LCMS: 616.4 [M+H]⁺.

Step 3: Preparation of tert-butyl (2-(1-(3-((tert-butoxycarbonyl)amino)propoxy)ethyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (3-(1-(4-((1-(tert-butyl)-3-((1S,3R)-3-((tert-butyldimethylsilyl)oxy)cyclopentyl)-1H-pyrazol-5-yl)amino)pyridin-2-yl)ethoxy)propyl)carbamate (1.13 g, 1.83 mmol, 1 eq) in DCM (20 mL) was added Boc₂O (1.20 g, 5.50 mmol, 1.26 mL, 3 eq), DIEA (711.35 mg, 5.50 mmol, 3 eq) and DMAP (112.07 mg, 0.917 mmol, 0.5 eq), and the mixture was stirred at 20° C. for 12 hours under N₂ atmosphere. The volatiles were then removed under reduced pressure, and the remaining residue was diluted water (50 mL) and extracted with ethyl acetate (50 mL×2). The combined organic layers were washed with brine (50 mL), dried over Na₂SO₄, filtered, and the filtrate was concentrated under reduced pressure to give tert-butyl (2-(1-(3-((tert-butoxycarbonyl)amino)propoxy)ethyl)pyridin-4-yl)(1-(tert-butyl)-3-((1S,3R)-3-((tert-butyldimethylsilyl)oxy)cyclopentyl)-1H-pyrazol-5-yl)carbamate (1.0 g, 1.40 mmol, 76.1% yield) as a yellow solid. LCMS: 716.5 [M+H]⁺.

Step 4: Preparation of tert-butyl (2-(1-(3-((tert-butoxycarbonyl)amino)propoxy)ethyl)pyridin-4-yl)(1-(tert-butyl)-3-((1S,3R)-3-hydroxycyclopentyl)-1H-pyrazol-5-yl)carbamate

The solution of tert-butyl (2-(1-(3-((tert-butoxycarbonyl)amino)propoxy)ethyl)pyridin-4-yl)(1-(tert-butyl)-3-((1S,3R)-3-((tert-butyldimethylsilyl)oxy)cyclopentyl)-1H-pyrazol-5-yl)carbamate (1.0 g, 1.40 mmol, 1 eq) and TBAF (1 M, 13.97 mL, 10 eq) in THF (2 mL) was stirred at 40° C. for 2 hours under N₂ atmosphere. After the completion of the reaction, the reaction mixture was concentrated and diluted with water (50 mL) and extracted with ethyl acetate (50 mL×2). The combined organic layers were washed with brine (50 mL), dried over Na₂SO₄, filtered, and the filtrate was concentrated under reduced pressure. The desired tert-butyl (2-(1-(3-((tert-butoxycarbonyl)amino)propoxy)ethyl)pyridin-4-yl)(1-(tert-butyl)-3-((1S,3R)-3-hydroxycyclopentyl)-1H-pyrazol-5-yl)carbamate (820 mg, 1.36 mmol, 97.6% yield) was obtained as a colorless oil. LCMS: 602.3 [M+H]⁺.

Step 5: Preparation of tert-butyl (2-(1-(3-((tert-butoxycarbonyl)amino)propoxy)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(1-(3-((tert-butoxycarbonyl)amino)propoxy)ethyl)pyridin-4-yl)(1-(tert-butyl)-3-((1S,3R)-3-hydroxycyclopentyl)-1H-pyrazol-5-yl)carbamate (400 mg, 0.664 mmol, 1 eq) in DCM (15 mL) was added Pyridine (315.47 mg, 3.99 mmol, 6 eq), (4-nitrophenyl) carbonochloridate (803.88 mg, 3.99 mmol, 6 eq) and DMAP (40.60 mg, 0.332 mmol, 0.5 eq), and the mixture was stirred at 50° C. for 16 hours under N₂ atmosphere. After the completion of the reaction, the reaction mixture was cooled to room temperature and the volatiles were removed. The remaining residue was diluted with water (50 mL) and extracted with ethyl acetate (50 mL×2). The combined organic layers were washed with brine (50 mL), dried over Na₂SO₄, filtered and the filtrate was concentrated under reduced pressure. The resulting residue was purified by prep-HPLC (TFA as additive) to afford tert-butyl (2-(1-(3-((tert-butoxycarbonyl)amino)propoxy)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate (300 mg, 0.391 mmol, 58.8% yield) as a yellow oil. LCMS: 767.4 [M+H]⁺.

Step 6: Preparation of (1R,3S)-3-(5-((2-(1-(3-aminopropoxy)ethyl)pyridin-4-yl)amino)-1-(tert-butyl)-1H-pyrazol-3-yl)cyclopentyl (4-nitrophenyl) carbonate

The solution of tert-butyl (2-(1-(3-((tert-butoxycarbonyl)amino)propoxy)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate (270 mg, 0.352 mmol, 1 eq) and TFA (401.5 mg, 3.52 mmol, 10 eq) in DCM (5 mL) was stirred at 20° C. for 3 hours under N₂ atmosphere. The volatiles were then removed under reduced pressure and the desired crude (1R,3S)-3-(5-((2-(1-(3-aminopropoxy)ethyl)pyridin-4-yl)amino)-1-(tert-butyl)-1H-pyrazol-3-yl)cyclopentyl (4-nitrophenyl) carbonate (190 mg, 95.2% yield) was obtained as a yellow solid. LCMS: 567.3 [M+H]⁺.

Step 7: Preparation of (1¹S,1³R,Z)-2¹-(tert-butyl)-5-methyl-2¹H-6,12-dioxa-3,10-diaza-4(4,2)-pyridina-2(3,5)-pyrazola-1 (1,3)-cyclopentanacyclododecaphan-11-one

To a solution of (1R,3S)-3-(5-((2-(1-(3-aminopropoxy)ethyl)pyridin-4-yl)amino)-1-(tert-butyl)-1H-pyrazol-3-yl)cyclopentyl (4-nitrophenyl) carbonate (180 mg, 0.317 mmol, 1 eq) in THF (50 mL) was added DIEA (410.55 mg, 3.18 mmol, 10 eq), and the mixture was stirred at 100° C. for 3 hours under N₂ atmosphere. After the completion of the reaction, the mixture was cooled to room temperature. The reaction mixture was diluted with H₂O (50 mL) and extracted with EtOAc (50 mL×3). The combined organic layers were washed with brine (50 mL), dried over Na₂SO₄, filtered, and the filtrate was concentrated under reduced pressure to afford (1¹S,1³R,Z)-2¹-(tert-butyl)-5-methyl-2¹H-6,12-dioxa-3,10-diaza-4(4,2)-pyridina-2(3,5)-pyrazola-1(1,3)-cyclopentanacyclododecaphan-11-one (135 mg, 87.5% yield) as a light-yellow oil. LCMS: 428.3 [M+H]⁺.

Step 8: Preparation of (1¹S,1³R,Z)-5-methyl-2¹H-6,12-dioxa-3,10-diaza-4(4,2)-pyridina-2(3,5)-pyrazola-1(1,3)-cyclopentanacyclododecaphan-11-one

A solution of (1¹S,1³R,Z)-2¹-(tert-butyl)-5-methyl-2¹H-6,12-dioxa-3,10-diaza-4(4,2)-pyridina-2(3,5)-pyrazola-1(1,3)-cyclopentanacyclododecaphan-11-one (120 mg, 0.28 mmol, 1 eq) in HCOOH (2 mL) was stirred at 25° C. for 3 hours under N₂ atmosphere. After the completion of the reaction, the reaction mixture was concentrated to afford (1¹S,1³R,Z)-5-methyl-2¹H-6,12-dioxa-3,10-diaza-4(4,2)-pyridina-2(3,5)-pyrazola-1(1,3)-cyclopentanacyclododecaphan-11-one (50 mg, 0.134 mmol, 48.0% yield, 100% purity) as a light-yellow oil, which was used to the next step without further purification. LCMS: 372.2 [M+H]⁺.

Step 9: Preparation of (1¹S,1³R,5R,Z)-5-methyl-2¹H-6,12-dioxa-3,10-diaza-4(4,2)-pyridina-2(3,5)-pyrazola-1(1,3)-cyclopentanacyclododecaphan-11-one and (1¹S,1³R,5S,Z)-5-methyl-2¹H-6,12-dioxa-3,10-diaza-4(4,2)-pyridina-2(3,5)-pyrazola-1(1,3)-cyclopentanacyclododecaphan-11-one

The resulting residue was purified by SFC column: DAICEL CHIRALPAK AS(250 mm*30 mm, 10 um); mobile phase: [CO₂-EtOH/ACN]; B %:45%, isocratic elution mode) to afford (1¹S,1³R,5R,Z)-5-methyl-2¹H-6,12-dioxa-3,10-diaza-4(4,2)-pyridina-2(3,5)-pyrazola-1(1,3)-cyclopentanacyclododecaphan-11-one (19.83 mg, 39.3% yield, 99.0% purity) and (1¹S,1³R,5S,Z)-5-methyl-2¹H-6,12-dioxa-3,10-diaza-4(4,2)-pyridina-2(3,5)-pyrazola-1(1,3)-cyclopentanacyclododecaphan-11-one (14.65 mg, 0.039 mmol, 29.0% yield, 99.0% purity) as a white solid.

(1¹S,1³R,5R,Z)-5-methyl-2¹H-6,12-dioxa-3,10-diaza-4(4,2)-pyridina-2(3,5)-pyrazola-1(1,3)-cyclopentanacyclododecaphan-11-one: LCMS: 372.2 [M+H]⁺; ¹H NMR (400 MHz, DMSO-d₆) δ 12.09-12.06 (m, 1H), 8.96 (s, 1H) 8.03-8.02 (m, 1H), 6.99 (s, 1H), 6.72-6.66 (m, 1H), 5.94 (s, 1H), 5.06 (s, 1H), 4.28-4.25 (m, 1H), 3.33-3.28 (m, 1H), 3.27-3.20 (m, 3H), 2.51-2.50 (m, 1H), 1.79-1.77 (m, 1H), 1.76-1.74 (m, 1H), 1.73-1.64 (m, 1H), 1.63-1.26 (m, 6H), 1.25-0.85 (m, 3H) ppm.

(1¹S,1³R,5S,Z)-5-methyl-2¹H-6,12-dioxa-3,10-diaza-4(4,2)-pyridina-2(3,5)-pyrazola-1(1,3)-cyclopentanacyclododecaphan-11-one: LCMS: 372.2 [M+H]⁺; ¹H NMR (400 MHz, DMSO-d₆) δ 12.14-12.13 (m, 1H), 8.93-8.83 (m, 1H), 8.03-8.02 (m, 1H), 7.00 (s, 1H), 6.83-6.65 (m, 2H), 5.94 (s, 1H), 507-4.99 (m, 1H), 4.29 (s, 1H), 3.49-3.26 (m, 1H), 3.24-3.23 (m, 2H), 2.87 (s, 1H), 2.50 (s, 1H), 2.08-2.04 (m, 1H), 2.02-1.75 (m, 7H), 1.73-1.26 (m, 3H) ppm.

Example 18: synthesis of (1¹S,1³R,7¹R,7³S,Z)-2¹H-5,10-dioxa-3,8-diaza-4(4,2)-pyridina-2(3,5)-pyrazola-1(1,3)-cyclopentana-7(1,3)-cyclobutanacyclodecaphan-9-one (compound 108)

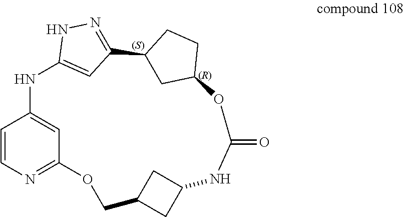

Step 1: Preparation of tert-butyl ((1r,3r)-3-(((4-bromopyridin-2-yl)oxy)methyl)cyclobutyl)carbamate

To a solution of 4-bromo-2-fluoro-pyridine (1.05 g, 5.96 mmol, 1.2 eq) and tert-butyl ((1r,3r)-3-(hydroxymethyl)cyclobutyl)carbamate (1 g, 4.97 mmol, 1 eq) in THF (15 mL) was added tBuOK (836.31 mg, 7.45 mmol, 1.5 eq), and the mixture was stirred at 25° C. for 2 hours under N₂ atmosphere. After the starting material was consumed, the mixture was poured into H₂O (30 mL) and extracted with EtOAc (50 mL×3). The combined organic layers were washed with brine (60 mL), dried over Na₂SO₄, filtered and the filtrate was concentrated in vacuo to afford 1.6 g of tert-butyl ((1r,3r)-3-(((4-bromopyridin-2-yl)oxy)methyl)cyclobutyl)carbamate as a white solid, which was used into the next step without further purification. LCMS: 357.0, 359.0 [M+H]⁺.

Step 2: Preparation of tert-butyl ((1S,3r)-3-(((4-((1-(tert-butyl)-3-((1S,3R)-3-((tert-butyldimethylsilyl)oxy)cyclopentyl)-1H-pyrazol-5-yl)amino)pyridin-2-yl)oxy)methyl)cyclobutyl)carbamate

A mixture of tert-butyl ((1r,3r)-3-(((4-bromopyridin-2-yl)oxy)methyl)cyclobutyl)carbamate (300 mg, 0.839 mmol, 1 eq), 1-(tert-butyl)-3-((1S,3R)-3-((tert-butyldimethylsilyl)oxy)cyclopentyl)-1H-pyrazol-5-amine (283.48 mg, 0.839 mmol, 1 eq), Pd₂(dba)₃ (76.90 mg, 0.084 mmol, 0.1 eq), Xantphos (80.07 mg, 0.168 mmol, 0.2 eq) and Cs₂CO₃ (820.84 mg, 2.52 mmol, 3 eq) in dioxane (3 mL) was stirred at 90° C. for 12 hours under N₂ atmosphere. After the completion of the reaction, the mixture was cooled to room temperature and poured into H₂O (50 mL). The aqueous solution was extracted with EtOAc (50 mL×3). The combined organic layers were washed with brine (60 mL), dried over Na₂SO₄, filtered and the filtrate was concentrated under vacuum. The resulting residue was purified by prep-TLC (PE:EA=3:1) to afford tert-butyl ((1S,3r)-3-(((4-((1-(tert-butyl)-3-((1S,3R)-3-((tert-butyldimethylsilyl)oxy)cyclopentyl)-1H-pyrazol-5-yl)amino)pyridin-2-yl)oxy)methyl)cyclobutyl)carbamate (121 mg, 23.5% yield) as a yellow oil. LCMS: 614.6 [M+H]⁺.

Step 3: Preparation of tert-butyl (2-(((1r,3S)-3-((tert-butoxycarbonyl)amino)cyclobutyl)methoxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((1S,3r)-3-(((4-((1-(tert-butyl)-3-((1S,3R)-3-((tert-butyldimethylsilyl)oxy)cyclopentyl)-1H-pyrazol-5-yl)amino)pyridin-2-yl)oxy)methyl)cyclobutyl)carbamate (121 mg, 0.20 mmol, 1 eq) in DCM (1 mL) was added Boc₂O (45.60 mg, 0.21 mmol, 1.06 eq) and DMAP (2.53 mg, 0.020 mmol, 0.1 eq), and the mixture was stirred at 25° C. for 1 hour. After the completion of the reaction, the mixture was poured into H₂O (10 mL) and extracted with DCM (5 mL×3). The combined organic layers were washed with brine (10 mL), dried over Na₂SO₄, filtered and the filtrate was concentrated to afford tert-butyl (2-(((1r,3S)-3-((tert-butoxycarbonyl)amino)cyclobutyl)methoxy)pyridin-4-yl)(1-(tert-butyl)-3-((1S,3R)-3-((tert-butyldimethylsilyl)oxy)cyclopentyl)-1H-pyrazol-5-yl)carbamate (117 mg, 0.16 mmol) as a white solid, which was used in the next step without further purification. LCMS: 714.5 [M+H]⁺.

Step 4: Preparation of tert-butyl (2-(((1r,3S)-3-((tert-butoxycarbonyl)amino)cyclobutyl)methoxy)pyridin-4-yl)(1-(tert-butyl)-3-((1S,3R)-3-hydroxycyclopentyl)-1H-pyrazol-5-yl)carbamate

The solution of tert-butyl (2-(((1r,3S)-3-((tert-butoxycarbonyl)amino)cyclobutyl)methoxy)pyridin-4-yl)(1-(tert-butyl)-3-((1S,3R)-3-((tert-butyldimethylsilyl)oxy)cyclopentyl)-1H-pyrazol-5-yl)carbamate (117 mg, 0.163 mmol, 1 eq) and TBAF (1 M, 0.66 mL, 4 eq) in THF (1 mL) was stirred at 25° C. for 16 hours. The mixture was then poured into H₂O (5 mL) and extracted with EtOAc (5 mL×3). The combined organic layers were washed with brine (20 mL), dried over Na₂SO₄, filtered and the filtrate was concentrated under vacuum. The resulting residue was purified by prep-TLC (PE:EA=1:1) to afford tert-butyl (2-(((1r,3S)-3-((tert-butoxycarbonyl)amino)cyclobutyl)methoxy)pyridin-4-yl)(1-(tert-butyl)-3-((1S,3R)-3-hydroxycyclopentyl)-1H-pyrazol-5-yl)carbamate (82 mg, 0.14 mmol, 83.4% yield) as a yellow oil, which was used in the next step without further purification. LCMS: 600.4 [M+H]⁺.

Step 5: Preparation of tert-butyl (2-(((1r,3S)-3-((tert-butoxycarbonyl)amino)cyclobutyl)methoxy)pyridin-4-yl)(1-(tert-butyl)-3-((1S,3R)-3-(((4-nitrophenoxy)carbonyl)oxy)cyclopentyl)-1H-pyrazol-5-yl)carbamate

To the solution of tert-butyl (2-(((1r,3S)-3-((tert-butoxycarbonyl)amino)cyclobutyl)methoxy)pyridin-4-yl)(1-(tert-butyl)-3-((1S,3R)-3-hydroxycyclopentyl)-1H-pyrazol-5-yl)carbamate (82 mg, 0.14 mmol, 1 eq) in DCM (1 mL) was added DMAP (8.35 mg, 0.068 mmol, 0.5 eq) and pyridine (64.89 mg, 0.82 mmol, 6 eq), followed by the addition of a solution of (4-nitrophenyl) carbonochloridate (82.67 mg, 0.41 mmol, 3 eq) in DCM (1 mL). After the completion of the addition, the mixture was stirred at 20° C. for 12 hours, and then concentrated in vacuo. The resulting residue was purified by prep-HPLC (FA) to afford tert-butyl (2-(((1r,3S)-3-((tert-butoxycarbonyl)amino)cyclobutyl)methoxy)pyridin-4-yl)(1-(tert-butyl)-3-((1S,3R)-3-(((4-nitrophenoxy)carbonyl)oxy)cyclopentyl)-1H-pyrazol-5-yl)carbamate (0.045 g, 41.1% yield) as a light-yellow oil. LCMS: 765.2 [M+H]⁺.

Step 6: Preparation of (1R,3S)-3-(5-((2-(((1r,3S)-3-aminocyclobutyl)methoxy)pyridin-4-yl)amino)-1-(tert-butyl)-1H-pyrazol-3-yl)cyclopentyl (4-nitrophenyl) carbonate

The solution of tert-butyl (2-(((1r,3S)-3-((tert-butoxycarbonyl)amino)cyclobutyl)methoxy)pyridin-4-yl)(1-(tert-butyl)-3-((1S,3R)-3-(((4-nitrophenoxy)carbonyl)oxy)cyclopentyl)-1H-pyrazol-5-yl)carbamate (70 mg, 0.091 mmol, 1 eq) and TFA (1.54 g, 13.46 mmol, 147.1 eq) in DCM (1 mL) was stirred at 25° C. for 12 hours. After the de-Boc reaction was completed, the mixture was concentrated to afford (1R,3S)-3-(5-((2-(((1r,3S)-3-aminocyclobutyl)methoxy)pyridin-4-yl)amino)-1-(tert-butyl)-1H-pyrazol-3-yl)cyclopentyl (4-nitrophenyl) carbonate (48 mg, 0.085 mmol) as a white solid, which was used in the next step without further purification. LCMS: 565.4 [M+H]⁺.

Step 7: Preparation of (1¹S,1³R,7¹R,7³S,Z)-2¹-(tert-butyl)-2¹H-5,10-dioxa-3,8-diaza-4(4,2)-pyridina-2(3,5)-pyrazola-1(1,3)-cyclopentana-7(1,3)-cyclobutanacyclodecaphan-9-one

To the solution of (1R,3S)-3-(5-((2-(((1r,3S)-3-aminocyclobutyl)methoxy)pyridin-4-yl)amino)-1-(tert-butyl)-1H-pyrazol-3-yl)cyclopentyl (4-nitrophenyl) carbonate (48 mg, 0.085 mmol, 1 eq) in THF (70 mL) was added DIEA (54.94 mg, 0.425 mmol, 5 eq), and the mixture was stirred at 70° C. for 12 hours. After the completion of the reaction, the mixture was cooled to room temperature and concentrated to afford (1¹S,1³R,7¹R,7³S,Z)-2¹-(tert-butyl)-2¹H-5,10-dioxa-3,8-diaza-4(4,2)-pyridina-2(3,5)-pyrazola-1(1,3)-cyclopentana-7(1,3)-cyclobutanacyclodecaphan-9-one (0.035 g, 96.7% yield) as a light-yellow solid, which was used in the next step without further purification. LCMS: 426.2 [M+H]⁺.

Step 8: Preparation of (1¹S,1³R,7¹R,7³S,Z)-2¹H-5,10-dioxa-3,8-diaza-4(4,2)-pyridina-2(3,5)-pyrazola-1(1,3)-cyclopentana-7(1,3)-cyclobutanacyclodecaphan-9-one

The solution of (1¹S,1³R,7¹R,7³S,Z)-2¹-(tert-butyl)-2¹H-5,10-dioxa-3,8-diaza-4(4,2)-pyridina-2(3,5)-pyrazola-1(1,3)-cyclopentana-7(1,3)-cyclobutanacyclodecaphan-9-one (0.035 g, 0.082 mmol, 1 eq) in formic acid (1 mL) was stirred at 100° C. for 1 hour under N₂ atmosphere. After the completion of the reaction, the mixture was cooled to room temperature and concentrated in vacuo. The resulting residue was purified by prep-HPLC (column: Waters Xbridge 150*25 mm*5 um; mobile phase: [water (ammonia hydroxide v/v)-ACN]; gradient:12%-42% B over 10 min) to afford (1¹S,1³R,7¹R,7³S,Z)-2¹H-5,10-dioxa-3,8-diaza-4(4,2)-pyridina-2(3,5)-pyrazola-1(1,3)-cyclopentana-7(1,3)-cyclobutanacyclodecaphan-9-one (24.89 mg, 81.6% yield) as a white solid. LCMS: 370.1 [M+H]⁺; ¹H NMR (400 MHz, DMSO-d₆) 5=12.07 (br, 1H), 8.97 (s, 1H), 7.75 (d, J=6.0 Hz, 1H), 7.04 (d, J=9.0 Hz, 1H), 6.48-6.43 (m, 1H), 6.05 (s, 1H), 5.13 (s, 1H), 5.13-5.10 (m, 1H), 4.58-4.55 (m, 1H), 4.31-4.21 (m, 2H), 3.22 (s, 1H), 2.13-2.09 (m, 1H), 2.05-1.96 (m, 3H), 1.95-1.90 (m, 3H), 1.74-1.68 (m, 3H) ppm.

Example 19: Synthesis of (1¹S,1³R,6R,Z)-6,9,9-trimethyl-2¹H-5,12-dioxa-3,10-diaza-4(4,2)-pyridina-2(3,5)-pyrazola-1(1,3)-cyclopentanacyclododecaphan-11-one (compound 112) and (1¹S,1³R,6S,Z)-6,9,9-trimethyl-2¹H-5,12-dioxa-3,10-diaza-4(4,2)-pyridina-2(3,5)-pyrazola-1(1,3)-cyclopentanacyclododecaphan-11-one (compound 113)

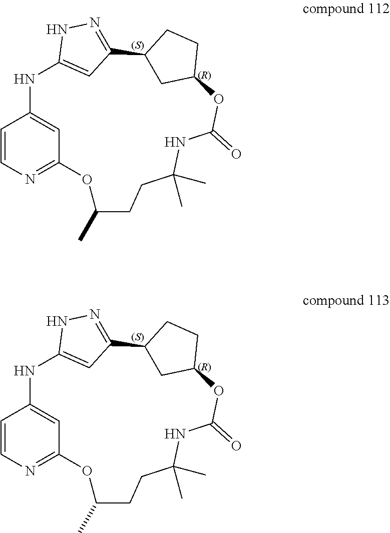

Step 1: Preparation of 5-methyl-5-nitrohexan-2-ol

To a solution of 4-methyl-4-nitropentanal (500 mg, 3.44 mmol, 1 eq) in DCM (10 mL) was added AlMe3 (2 M, 3.44 mL, 2 eq) at 0° C. under N₂ atmosphere. After the completion of the addition, the mixture was kept stirring at 0° C. for 1 hour, and then warmed to 20° C. for an additional 12 hours under N₂ atmosphere. The reaction was then quenched with HCl (1N, 20 mL), and the mixture was extracted with EtOAc (20 mL×3). The combined organic phase was washed with brine (30 mL), dried over Na₂SO₄, filtered and the filtrate was concentrated under reduced pressure. The desired 5-methyl-5-nitrohexan-2-ol (800 mg) was obtained as a yellow oil, which was used in the next step without further purification. ¹HNMR (400 MHz, CHLOROFORM-d) δ 3.77-3.67 (m, 1H), 2.02-1.96 (m, 1H), 1.92-1.82 (m, 1H), 1.52 (d, J=2.0 Hz, 6H), 1.38-1.30 (m, 2H), 1.14 (d, J=6.0 Hz, 3H) ppm.

Step 2: Preparation of 4-bromo-2-((5-methyl-5-nitrohexan-2-yl)oxy)pyridine)pyridine

To a solution of 5-methyl-5-nitrohexan-2-ol (500 mg, 3.10 mmol, 1 eq) and 4-bromo-2-fluoro-pyridine (545.87 mg, 3.10 mmol, 1 eq) in DMF (10 mL) was added t-BuOK (522.08 mg, 4.65 mmol, 1.5 eq) at 0° C. under N₂ atmosphere, and the mixture was stirred for 1 hour at that temperature until the completion of the reaction. The reaction was quenched with saturated aqueous NH₄Cl (20 mL), and the mixture was extracted with EtOAc (20 mL×3). The combined organic phase was dried over Na₂SO₄, filtered and the filtrate was concentrated under reduced pressure. The resulting residue was purified by column chromatography to give 4-bromo-2-((5-methyl-5-nitrohexan-2-yl)oxy)pyridine (800 mg, 81.3% yield) as a yellow oil. LCMS: 317.0, 319.0 [M+H]+

Step 3: Preparation of N-(1-(tert-butyl)-3-((1S,3R)-3-((tert-butyldimethylsilyl)oxy)cyclopentyl)-1H-pyrazol-5-yl)-2-((5-methyl-5-nitrohexan-2-yl)oxy)pyridin-4-amine

The mixture of 1-(tert-butyl)-3-((1S,3R)-3-((tert-butyldimethylsilyl)oxy)cyclopentyl)-1H-pyrazol-5-amine (425.72 mg, 1.26 mmol, 1 eq), 4-bromo-2-((5-methyl-5-nitrohexan-2-yl)oxy)pyridine (400 mg, 1.26 mmol, 1 eq), Pd₂(dba)₃ (115.48 mg, 0.13 mmol, 0.1 eq), Xantphos (145.94 mg, 0.25 mmol, 0.2 eq) and Cs₂CO₃ (1.23 g, 3.78 mmol, 3 eq) in dioxane (5 mL) was stirred at 90° C. for 1 hour under N₂ atmosphere. After the completion of the reaction, the mixture was cooled to room temperature, filtered through a short pad of celite and rinsed with ethyl acetate (50 mL). The combined filtrate was concentrated under reduced pressure and the resulting residue was purified by prep-TLC (PE/EA=3/1) to give N-(1-(tert-butyl)-3-((1S,3R)-3-((tert-butyldimethylsilyl)oxy)cyclopentyl)-1H-pyrazol-5-yl)-2-((5-methyl-5-nitrohexan-2-yl)oxy)pyridin-4-amine (400 mg, 69.8% yield) as a yellow oil. LCMS: 574.5 [M+H]⁺.

Step 4: Preparation of 2-((5-amino-5-methylhexan-2-yl)oxy)-N-(1-(tert-butyl)-3-((1S,3R)-3-((tert-butyldimethylsilyl)oxy)cyclopentyl)-1H-pyrazol-5-yl)pyridin-4-amine

A mixture of N-(1-(tert-butyl)-3-((1S,3R)-3-((tert-butyldimethylsilyl)oxy)cyclopentyl)-1H-pyrazol-5-yl)-2-((5-methyl-5-nitrohexan-2-yl)oxy)pyridin-4-amine (400 mg, 0.70 mmol, 1 eq) and Raney-Ni (249.6 mg, 20% purity) in MeOH (10 mL) was degassed and backfilled with H₂ for 3 times, and stirred at 60° C. for 12 hours under H₂ (50 Psi) atmosphere. After the starting material was consumed, the mixture was cooled to room temperature, filtered through a short pad of celite and rinsed with methanol (20 mL). The combined filtrate was concentrated under reduced pressure. The resulting residue was purified by prep-TLC (PE/EA=3/1) to give 2-((5-amino-5-methylhexan-2-yl)oxy)-N-(1-(tert-butyl)-3-((1S,3R)-3-((tert-butyldimethylsilyl)oxy)cyclopentyl)-1H-pyrazol-5-yl)pyridin-4-amine (400 mg) as a yellow oil. LCMS: 544.6 [M+H]⁺.

Step 5: Preparation of tert-butyl (5-((4-((1-(tert-butyl)-3-((1S,3R)-3-((tert-butyldimethylsilyl)oxy)cyclopentyl)-1H-pyrazol-5-yl)amino)pyridin-2-yl)oxy)-2-methylhexan-2-yl)carbamate

To a solution of 2-((5-amino-5-methylhexan-2-yl)oxy)-N-(1-(tert-butyl)-3-((1S,3R)-3-((tert-butyldimethylsilyl)oxy)cyclopentyl)-1H-pyrazol-5-yl)pyridin-4-amine (400 mg, 0.74 mmol, 1 eq) in DCM (10 mL) was added TEA (223.2 mg, 2.21 mmol, 3 eq) and Boc₂O (481.56 mg, 2.21 mmol, 3 eq), and the mixture was stirred at 25° C. for 12 hours under N₂ atmosphere. The mixture was then filtered, concentrated and purified by prep-TLC (PE/EA=3/1) to give tert-butyl (5-((4-((1-(tert-butyl)-3-((1S,3R)-3-((tert-butyldimethylsilyl)oxy)cyclopentyl)-1H-pyrazol-5-yl)amino)pyridin-2-yl)oxy)-2-methylhexan-2-yl)carbamate (450 mg, 93.1% yield) as a yellow gum. LCMS: 644.5 [M+H]⁺.

Step 6: Preparation of tert-butyl (2-((5-((tert-butoxycarbonyl)amino)-5-methylhexan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (5-((4-((1-(tert-butyl)-3-((1S,3R)-3-((tert-butyldimethylsilyl)oxy)cyclopentyl)-1H-pyrazol-5-yl)amino)pyridin-2-yl)oxy)-2-methylhexan-2-yl)carbamate (400 mg, 0.62 mmol, 1 eq) in DCM (5 mL) was added Boc₂O (271.1 mg, 1.24 mmol, 2 eq) and DMAP (75.89 mg, 0.62 mmol, 1 eq), and the mixture was stirred at 25° C. for 1 hour under N₂ atmosphere. The mixture was then filtered, and the filtrate was concentrated. The crude product was purified by prep-TLC (PE/EA=3/1) to give tert-butyl (2-((5-((tert-butoxycarbonyl)amino)-5-methylhexan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate (450 mg, 95.4% yield) as a yellow solid. LCMS: 744.6 [M+H]⁺.

Step 7: Preparation of tert-butyl (2-((5-((tert-butoxycarbonyl)amino)-5-methylhexan-2-yl)oxy)pyridin-4-yl)(1-(tert-butyl)-3-((1S,3R)-3-hydroxycyclopentyl)-1H-pyrazol-5-yl)carbamate

The solution of tert-butyl (2-((5-((tert-butoxycarbonyl)amino)-5-methylhexan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate (400 mg, 0.54 mmol, 1 eq) and TBAF (1 M in THF, 1.08 mL, 2 eq) in THF (5 mL) was stirred at 60° C. for 2 hours under N₂ atmosphere. After the starting material was consumed, the mixture was cooled to room temperature, diluted with H₂O (50 mL) and extracted with EtOAc (50 mL×2). The combined organic layers were washed with brine (50 mL×2), dried over anhydrous Na₂SO₄, filtered and the filtrate was concentrated in vacuo to afford tert-butyl (2-((5-((tert-butoxycarbonyl)amino)-5-methylhexan-2-yl)oxy)pyridin-4-yl)(1-(tert-butyl)-3-((1S,3R)-3-hydroxycyclopentyl)-1H-pyrazol-5-yl)carbamate (350 mg, 0.56 mmol) as a yellow oil, which was used in the next step without further purification. LCMS: 630.6 [M+H]⁺.

Step 8: Preparation of tert-butyl (2-((5-((tert-butoxycarbonyl)amino)-5-methylhexan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((5-((tert-butoxycarbonyl)amino)-5-methylhexan-2-yl)oxy)pyridin-4-yl)(1-(tert-butyl)-3-((1S,3R)-3-hydroxycyclopentyl)-1H-pyrazol-5-yl)carbamate (350 mg, 0.55 mmol, 1 eq) in THF (3 mL) were added Pyridine (219.78 mg, 2.78 mmol, 0.22 mL, 5 eq) and DMAP (33.95 mg, 0.28 mmol, 0.5 eq), followed by the addition of a solution of (4-nitrophenyl) carbonochloridate (336.03 mg, 1.67 mmol, 3 eq) in DCM (3 mL) under N₂ atmosphere. After the completion of the addition, the mixture was stirred at 25° C. for 12 hours under N₂ atmosphere. The mixture was filtered, and the filtrate was concentrated. The resulting crude was purified by prep-TLC (PE/EA=3/1) to give tert-butyl (2-((5-((tert-butoxycarbonyl)amino)-5-methylhexan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (420 mg, 96.2% yield) as a yellow solid. LCMS: 795.6 [M+H]⁺.

Step 9: Preparation of (1R,3S)-3-(5-((2-((5-amino-5-methylhexan-2-yl)oxy)pyridin-4-yl)amino)-1-(tert-butyl)-1H-pyrazol-3-yl)cyclopentyl (4-nitrophenyl) carbonate

The solution of tert-butyl (2-((5-((tert-butoxycarbonyl)amino)-5-methylhexan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (500 mg, 0.63 mmol, 1 eq) and TFA (4.7 mL) in DCM (5 mL) was stirred at 30° C. for 0.5 hour under N₂ atmosphere. After the completion of the reaction, the mixture was filtered, and the filtrate was concentrated to give (1R,3S)-3-(5-((2-((5-amino-5-methylhexan-2-yl)oxy)pyridin-4-yl)amino)-1-(tert-butyl)-1H-pyrazol-3-yl)cyclopentyl (4-nitrophenyl) carbonate (350 mg, 74.8% yield) as a yellow solid. LCMS: 595.4 [M+H]⁺.

Step 10: Preparation of (1¹S,1³R,Z)-2¹-(tert-butyl)-6,9,9-trimethyl-2¹H-5,12-dioxa-3,10-diaza-4(4,2)-pyridina-2(3,5)-pyrazola-1(1,3)-cyclopentanacyclododecaphan-11-one

To a solution of (1R,3S)-3-(5-((2-((5-amino-5-methylhexan-2-yl)oxy)pyridin-4-yl)amino)-1-(tert-butyl)-1H-pyrazol-3-yl)cyclopentyl (4-nitrophenyl) carbonate (300 mg, 0.50 mmol, 1 eq) in DMSO (1 mL) was added TEA (510.45 mg, 5.04 mmol, 0.70 mL, 10 eq), and the mixture was stirred at 100° C. for 0.5 hour under N₂ atmosphere. After the completion of the reaction, the mixture was cooled to room temperature, diluted with H₂O (50 mL) and extracted with EtOAc (30 mL×3). The combined organic phase was washed with brine (100 mL), dried over Na₂SO₄, filtered, and the filtrate was concentrated. The resulting residue was purified by prep-TLC (PE/EA=1/1) to give (1¹S,1³R,Z)-2¹-(tert-butyl)-6,9,9-trimethyl-2¹H-5,12-dioxa-3,10-diaza-4(4,2)-pyridina-2(3,5)-pyrazola-1 (1,3)-cyclopentanacyclododecaphan-11-one (90 mg, 39.6% yield) as a yellow solid. LCMS: 456.3[M+H]⁺.

Step 11: Preparation of (1¹S,1³R,Z)-6,9,9-trimethyl-2¹H-5,12-dioxa-3,10-diaza-4(4,2)-pyridina-2(3,5)-pyrazola-1(1,3)-cyclopentanacyclododecaphan-11-one

A solution of (1¹S,1³R,Z)-2¹-(tert-butyl)-6,9,9-trimethyl-2¹H-5,12-dioxa-3,10-diaza-4(4,2)-pyridina-2(3,5)-pyrazola-1(1,3)-cyclopentanacyclododecaphan-11-one (80 mg, 0.17 mmol, 1 eq) in formic acid (5 mL) was stirred at 100° C. for 0.5 hour under N₂ atmosphere. After the completion of the reaction, the mixture was cooled to room temperature and the volatiles were removed under reduced pressure. The resulting residue was purified by reversed-phase column (C18 150*40 mm*15 um, 0.1% FA) to give (1¹S,1³R,Z)-6,9,9-trimethyl-2¹H-5,12-dioxa-3,10-diaza-4(4,2)-pyridina-2(3,5)-pyrazola-1(1,3)-cyclopentanacyclododecaphan-11-one (50 mg, 67.7% yield) as a white solid. LCMS: 400.3 [M+H]⁺.

Step 12: Preparation of (1¹S,1³R,6R,Z)-6,9,9-trimethyl-2¹H-5,12-dioxa-3,10-diaza-4(4,2)-pyridina-2(3,5)-pyrazola-1(1,3)-cyclopentanacyclododecaphan-11-one and (1¹S,1³R,6S,Z)-6,9,9-trimethyl-2¹H-5,12-dioxa-3,10-diaza-4(4,2)-pyridina-2(3,5)-pyrazola-1(1,3)-cyclopentanacyclododecaphan-11-one

(1¹S,1³R,Z)-6,9,9-trimethyl-2¹H-5,12-dioxa-3,10-diaza-4(4,2)-pyridina-2(3,5)-pyrazola-1(1,3)-cyclopentanacyclododecaphan-11-one (50 mg, 0.13 mmol, 1 eq) was purified by SFC column: (DAICEL CHIRALPAK AD(250 mm*30 mm, 10 um); mobile phase: [CO₂-EtOH(0.1% NH₃H₂O)]; B %:40%, isocratic elution mode) to afford (1¹S,1³R,6R,Z)-6,9,9-trimethyl-2¹H-5,12-dioxa-3,10-diaza-4(4,2)-pyridina-2(3,5)-pyrazola-1(1,3)-cyclopentanacyclododecaphan-11-one (15.98 mg, 0.040 mmol, 31.4% yield) as a white solid and (1¹S,1³R,6S,Z)-6,9,9-trimethyl-2¹H-5,12-dioxa-3,10-diaza-4(4,2)-pyridina-2(3,5)-pyrazola-1(1,3)-cyclopentanacyclododecaphan-11-one (18.75 mg, 36.8% yield) as a white solid.

(1¹S,1³R,6R,Z)-6,9,9-trimethyl-2¹H-5,12-dioxa-3,10-diaza-4(4,2)-pyridina-2(3,5)-pyrazola-1(1,3)-cyclopentanacyclododecaphan-11-one LCMS: 400.2 [M+H]⁺; ¹H NMR (400 MHz, DMSO-d₆) δ 12.23-12.05 (m, 1H), 9.34-8.93 (m, 1H), 7.76 (d, J=6.0 Hz, 1H), 6.48 (br d, J=5.0 Hz, 1H), 6.40 (s, 1H), 6.29-6.19 (m, 1H), 6.08 (s, 1H), 5.20-5.10 (m, 1H), 4.85-4.73 (m, 1H), 3.26-3.19 (m, 1H), 1.98-1.79 (m, 4H), 1.77-1.61 (m, 4H), 1.57-1.44 (m, 2H), 1.34-1.27 (m, 3H), 1.25 (br d, J=5.2 Hz, 3H), 1.17 (s, 3H) ppm.

(1¹S,1³R,6S,Z)-6,9,9-trimethyl-2¹H-5,12-dioxa-3,10-diaza-4(4,2)-pyridina-2(3,5)-pyrazola-1(1,3)-cyclopentanacyclododecaphan-11-one LCMS: 400.2 [M+H]⁺; ¹H NMR (400 MHz, DMSO-d₆) δ 12.26-12.03 (m, 1H), 9.10-8.90 (m, 1H), 7.75 (d, J=6.4 Hz, 1H), 6.45 (dd, J=1.6, 6.0 Hz, 1H), 6.38 (d, J=1.6 Hz, 1H), 6.16-5.97 (m, 2H), 5.04-4.95 (m, 1H), 4.80-4.71 (m, 1H), 3.25-3.21 (m, 1H), 1.98-1.82 (m, 4H), 1.74-1.45 (m, 6H), 1.28-1.22 (m, 6H), 1.18 (br s, 3H) ppm.

Example 20: Synthesis of (1¹S,1³R,9S,Z)-9-methyl-2¹H-12-oxa-3,10-diaza-4(4,2)-pyridina-2(3,5)-pyrazola-1(1,3)-cyclopentanacyclododecaphan-11-one (compound 114)

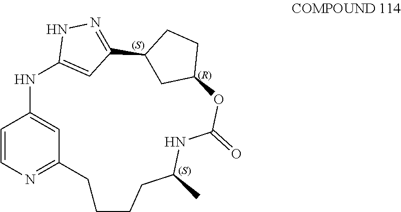

Step 1: Preparation of tert-butyl (S)-(6-(4-bromopyridin-2-yl)hex-5-yn-2-yl)carbamate

The mixture of tert-butyl (S)-hex-5-yn-2-ylcarbamate (190 mg, 0.96 mmol, 1 eq), 2,4-dibromopyridine (300 mg, 1.27 mmol, 1.3 eq), CuI (40 mg, 0.21 mmol, 0.22 eq), TEA (218.10 mg, 2.16 mmol, 0.3 mL, 2.24 eq) and Pd(PPh₃)₄ (167 mg, 0.14 mmol, 0.15 eq) in DMF (5 mL) was heated at 40° C. for 16 hours under N₂ atmosphere. The reaction was then cooled to room temperature. The mixture was diluted with water (100 mL) and extracted with ethyl acetate (100 mL×2). The combined organic phase was washed with saturated aqueous NaCl (50 mL), dried over Na₂SO₄, filtered and the filtrate was concentrated in vacuum. The resulting residue was purified by column chromatography to afford tert-butyl (S)-(6-(4-bromopyridin-2-yl)hex-5-yn-2-yl)carbamate (0.15 g, 44.1% yield) as a yellow oil. LCMS: 353.2 [M+H]⁺.

Step 2: Preparation of tert-butyl ((S)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-yn-2-yl)carbamate

The mixture of tert-butyl (S)-(6-(4-bromopyridin-2-yl)hex-5-yn-2-yl)carbamate (0.15 g, 0.42 mmol, 1 eq), 1-(tert-butyl)-3-((1S,3R)-3-((tert-butyldimethylsilyl)oxy)cyclopentyl)-1H-pyrazol-5-amine (0.15 g, 0.44 mmol, 1.05 eq), Pd₂(dba)₃ (388.8 mg, 0.42 mmol, 1 eq), Xantphos (245.7 mg, 0.42 mmol, 1 eq) and Cs₂CO₃ (138.3 mg, 0.42 mmol, 1 eq) in dioxane (5 mL) was degassed and backfilled with N₂ for 3 times and heated at 90° C. for 16 hours under N₂ atmosphere. The mixture was then cooled to room temperature, diluted with water (50 mL) and extracted with ethyl acetate (100 mL×2). The organic layers were washed with brine (50 mL), dried over Na₂SO₄, filtered and the filtrate was concentrated in vacuum. The resulting residue was purified by column chromatography to afford tert-butyl ((S)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-yn-2-yl)carbamate (0.13 g, 50.2% yield) as a yellow solid. LCMS: 610.6 [M+H]⁺.

Step 3: tert-butyl ((S)-6-(4-((1-(tert-butyl)-3-((1S,3R)-3-((tert-butyldimethylsilyl)oxy)cyclopentyl)-1H-pyrazol-5-yl)amino)pyridin-2-yl)hexan-2-yl)carbamate

The mixture of tert-butyl ((S)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-yn-2-yl)carbamate (130 mg, 0.21 mmol, 1 eq) and Pd(OAc)₂ (3.68 mg, 16.40 mmol, 1 eq) in THF (5 mL) was degassed and backfilled with H₂ for 3 times, and stirred under H₂ atmosphere at 20° C. for 2 hours. The reaction mixture was filtered, rinsed with THF (5 mL) and the filtrate was concentrated under reduced pressure to give the crude tert-butyl ((S)-6-(4-((1-(tert-butyl)-3-((1S,3R)-3-((tert-butyldimethylsilyl)oxy)cyclopentyl)-1H-pyrazol-5-yl)amino)pyridin-2-yl)hexan-2-yl)carbamate (90 mg, 0.15 mmol) as a black brown solid, which was used in the next step without further purification. LCMS: 614.6 [M+H]⁺.

Step 4: Preparation of tert-butyl (2-((S)-5-((tert-butoxycarbonyl)amino)hexyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-6-(4-((1-(tert-butyl)-3-((1S,3R)-3-((tert-butyldimethylsilyl)oxy)cyclopentyl)-1H-pyrazol-5-yl)amino)pyridin-2-yl)hexan-2-yl)carbamate (90 mg, 0.15 mmol, 1 eq) in DCM (2 mL) was added DMAP (17.9 mg, 0.15 mmol, 1 eq) and Boc₂O (64.0 mg, 0.29 mmol, 0.067 ml, 2 eq), and the mixture was stirred at 20° C. for 16 hours under N₂ atmosphere. After the starting material was consumed, the mixture was diluted with H₂O (10 mL) and extracted with DCM (10 mL×3). The combined organic layers were washed with brine (10 mL×2), dried over Na₂SO₄, filtered and the filtrate was concentrated under reduced pressure. The resulting residue was purified by prep-TLC (Petroleum ether/Ethyl acetate=3/1) to afford tert-butyl (2-((S)-5-((tert-butoxycarbonyl)amino)hexyl)pyridin-4-yl)(1-(tert-butyl)-3-((1S,3R)-3-((tert-butyldimethylsilyl)oxy)cyclopentyl)-1H-pyrazol-5-yl)carbamate (64 mg, 61.1% yield) as a deep brown solid. LCMS: 714.7 [M+H]⁺.

Step 5: Preparation of tert-butyl (2-((S)-5-((tert-butoxycarbonyl)amino)hexyl)pyridin-4-yl)(1-(tert-butyl)-3-((1S,3R)-3-hydroxycyclopentyl)-1H-pyrazol-5-yl)carbamate

The solution of tert-butyl (2-((S)-5-((tert-butoxycarbonyl)amino)hexyl)pyridin-4-yl)(1-(tert-butyl)-3-((1S,3R)-3-((tert-butyldimethylsilyl)oxy)cyclopentyl)-1H-pyrazol-5-yl)carbamate (64 mg, 0.090 mmol, 1 eq) and TBAF (1 M, 0.45 mL, 5 eq) in THF (1.5 mL) was stirred at 20° C. for 16 hours under N₂ atmosphere. After the completion of the reaction, the reaction mixture was concentrated under reduced pressure to give 42 mg of tert-butyl (2-((S)-5-((tert-butoxycarbonyl)amino)hexyl)pyridin-4-yl)(1-(tert-butyl)-3-((1S,3R)-3-hydroxycyclopentyl)-1H-pyrazol-5-yl)carbamate as a yellow oil, which was used in the next step without further purification. LCMS: 600.4 [M+H]⁺.

Step 6: Preparation of tert-butyl (2-((S)-5-((tert-butoxycarbonyl)amino)hexyl)pyridin-4-yl)(1-(tert-butyl)-3-((1S,3R)-3-(((4-nitrophenoxy)carbonyl)oxy)cyclopentyl)-1H-pyrazol-5-yl)carbamate

The mixture of tert-butyl (2-((S)-5-((tert-butoxycarbonyl)amino)hexyl)pyridin-4-yl)(1-(tert-butyl)-3-((1S,3R)-3-hydroxycyclopentyl)-1H-pyrazol-5-yl)carbamate (42 mg, 0.070 mmol, 1 eq), (4-nitrophenyl) carbonochloridate (42.34 mg, 0.210 mmol, 3 eq), pyridine (33.23 mg, 0.420 mmol, 0.033 ml, 6 eq) and DMAP (8.55 mg, 0.070 mmol, 1 eq) in DCM (3 mL) was stirred at 20° C. for 4 hours under N₂ atmosphere. After the completion of the reaction, the reaction mixture was diluted with H₂O (10 mL) and extracted with ethyl acetate (10 mL×3). The combined organic layers were washed with brine (10 mL×2), dried over Na₂SO₄, filtered and the filtrate was concentrated under reduced pressure. The resulting residue was purified by column chromatography to afford tert-butyl (2-((S)-5-((tert-butoxycarbonyl)amino)hexyl)pyridin-4-yl)(1-(tert-butyl)-3-((1S,3R)-3-(((4-nitrophenoxy)carbonyl)oxy)cyclopentyl)-1H-pyrazol-5-yl)carbamate (43 mg, 80.83% yield) as a yellow oil. LCMS: 765.5 [M+H]⁺.

Step 7: Preparation of (1R,3S)-3-(5-((2-((S)-5-aminohexyl)pyridin-4-yl)amino)-1-(tert-butyl)-1H-pyrazol-3-yl)cyclopentyl (4-nitrophenyl) carbonate

The solution of tert-butyl (2-((S)-5-((tert-butoxycarbonyl)amino)hexyl)pyridin-4-yl)(1-(tert-butyl)-3-((1S,3R)-3-(((4-nitrophenoxy)carbonyl)oxy)cyclopentyl)-1H-pyrazol-5-yl)carbamate (43 mg, 0.056 mmol, 1 eq) and TFA (38.5 mg, 0.34 mmol, 0.025 ml, 6 eq) in DCM (2 mL) was stirred at 20° C. for 16 hours under N₂ atmosphere. After the completion of the reaction, the reaction mixture was concentrated under reduced pressure to give 26 mg of (1R,3S)-3-(5-((2-((S)-5-aminohexyl)pyridin-4-yl)amino)-1-(tert-butyl)-1H-pyrazol-3-yl)cyclopentyl (4-nitrophenyl) carbonate as a yellow oil, which was used in the next step without further purification. LCMS: 565.5 [M+H]⁺.

Step 8: Preparation of (1¹S,1³R,9S,Z)-2¹-(tert-butyl)-9-methyl-2¹H-12-oxa-3,10-diaza-4(4,2)-pyridina-2(3,5)-pyrazola-1(1,3)-cyclopentanacyclododecaphan-11-one

To a solution of (1R,3S)-3-(5-((2-((S)-5-aminohexyl)pyridin-4-yl)amino)-1-(tert-butyl)-1H-pyrazol-3-yl)cyclopentyl (4-nitrophenyl) carbonate (26 mg, 0.046 mmol, 1 eq) in THF (50 mL) was added DIEA (29.75 mg, 0.23 mmol, 0.040 ml, 5 eq), and the mixture was stirred at 20° C. for 4 hours under N₂ atmosphere. The reaction mixture was then concentrated under reduced pressure to give (1¹S,1³R,9S,Z)-2¹-(tert-butyl)-9-methyl-2¹H-12-oxa-3,10-diaza-4(4,2)-pyridina-2(3,5)-pyrazola-1(1,3)-cyclopentanacyclododecaphan-11-one (18 mg, 91.9% yield) as a yellow oil, which was used in the next step without further purification. LCMS: 426.2 [M+H]⁺.

Step 9: Preparation of (1¹S,1³R,9S,Z)-9-methyl-2¹H-12-oxa-3,10-diaza-4(4,2)-pyridina-2(3,5)-pyrazola-1(1,3)-cyclopentanacyclododecaphan-11-one

A solution of (1¹S,1³R,9S,Z)-2¹-(tert-butyl)-9-methyl-2¹H-12-oxa-3,10-diaza-4(4,2)-pyridina-2(3,5)-pyrazola-1(1,3)-cyclopentanacyclododecaphan-11-one (18 mg, 0.042 mmol, 1 eq) in formic acid (3 mL) was stirred at 100° C. for 12 hours under N₂ atmosphere. The mixture was then cooled to room temperature and concentrated under reduced pressure. The resulting residue was purified by prep-HPLC (column: Phenomenex luna C18 150×25 mm×10 um; mobile phase: [water (FA)-ACN]; gradient:5%-35% B over 10 min) to afford the desired (1¹S,1³R,9S,Z)-9-methyl-2¹H-12-oxa-3,10-diaza-4(4,2)-pyridina-2(3,5)-pyrazola-1(1 3)-cyclopentanacyclododecaphan-11-one (11.78 mg, 72.9% yield) as an off-white solid. LCMS: 370.2 [M+H]⁺; ¹H NMR (400 MHz, DMSO-d₆) δ 9.16 (s, 1H), 8.17 (s, 1H), 8.06-8.04 (d, J=6.0 Hz, 1H), 6.94-6.92 (d, J=8.8 Hz, 1H), 6.76 (s, 1H), 6.68-6.67 (m, 1H), 5.95 (s, 1H), 4.99 (s, 1H), 3.62-3.58 (m, 2H), 3.22-3.20 (m, 2H), 2.68 (m, 1H), 2.47-2.45 (m, 1H), 2.05-2.03 (m, 1H), 1.82-1.76 (m, 4H), 1.45-1.23 (m, 6H), 0.98 (d, J=6.4 Hz, 3H) ppm.

Example 21: synthesis of (1¹S,1³R,9R,Z)-9-methyl-2¹H-12-oxa-3,10-diaza-4(4,2)-pyridina-2(3,5)-pyrazola-1(1,3)-cyclopentanacyclododecaphan-11-one (compound 115)

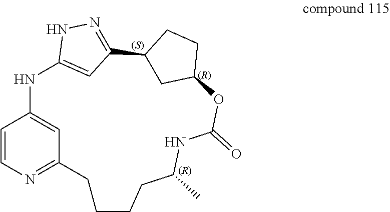

Step 1: Preparation of tert-butyl ((R)-6-(4-((1-(tert-butyl)-3-((1S,3R)-3-((tert-butyldimethylsilyl)oxy) cyclopentyl)-1H-pyrazol-5-yl)amino)pyridin-2-yl)hex-5-yn-2-yl)carbamate

A mixture of tert-butyl (R)-(6-(4-bromopyridin-2-yl)hex-5-yn-2-yl)carbamate (246 mg, 0.696 mmol, 1 eq), 1-(tert-butyl)-3-((1S,3R)-3-((tert-butyldimethylsilyl)oxy)cyclopentyl)-1H-pyrazol-5-amine (235.08 mg, 0.696 mmol, 1 eq), Pd₂(dba)₃ (63.77 mg, 0.07 mmol, 0.1 eq), Xantphos (80.59 mg, 0.014 mmol, 0.2 eq) and Cs₂CO₃ (680.69 mg, 2.09 mmol, 3 eq) in dioxane (15 mL) was degassed and backfilled with N₂ for 3 times, and stirred at 90° C. for 12 hours under N₂ atmosphere. After the completion of the reaction, the mixture was cooled to room temperature, diluted with H₂O (30 mL) and extracted with ethyl acetate (30 mL×2). The combined organic layers were washed with brine (30 mL), dried over Na₂SO₄, filtered and the filtrate was concentrated under reduced pressure. The resulting residue was purified by column chromatography to afford tert-butyl ((R)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-yn-2-yl)carbamate (380 mg, 89.5% yield) as a yellow solid. LCMS: 610.5 [M+H]⁺.

Step 2: Preparation of tert-butyl ((R)-6-(4-((1-(tert-butyl)-3-((1S,3R)-3-((tert-butyldimethylsilyl)oxy)cyclopentyl)-1H-pyrazol-5-yl)amino)pyridin-2-yl)hexan-2-yl)carbamate

A mixture of tert-butyl ((R)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-yn-2-yl)carbamate (350 mg, 0.574 mmol, 1 eq) and Pd/C (175.00 mg, 10% purity) in THF (5 mL) was degassed and backfilled with H₂ for 3 times, and the mixture was stirred at 40° C. for 1 hour under H₂ atmosphere. After the completion of the reaction, the mixture was cooled to room temperature, filtered through a short pad of celite and rinsed with THF (20 mL). The combined filtrate was concentrated in vacuo to afford tert-butyl ((R)-6-(4-((1-(tert-butyl)-3-((1S,3R)-3-((tert-butyldimethylsilyl)oxy)cyclopentyl)-1H-pyrazol-5-yl)amino)pyridin-2-yl)hexan-2-yl)carbamate (280 mg, 79.5% yield) as a yellow solid. LCMS: 614.5 [M+H]⁺.

Step 3: tert-butyl (2-((R)-5-((tert-butoxycarbonyl)amino)hexyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((R)-6-(4-((1-(tert-butyl)-3-((1S,3R)-3-((tert-butyldimethylsilyl)oxy)cyclopentyl)-1H-pyrazol-5-yl)amino)pyridin-2-yl)hexan-2-yl)carbamate (280 mg, 0.456 mmol, 1 eq) in DCM (20 mL) was added Boc₂O (199.07 mg, 0.912 mmol, 2 eq) and DMAP (55.72 mg, 0.456 mmol, 1 eq), and the mixture was stirred at 20° C. for 1 hour under N₂ atmosphere. The reaction mixture was then concentrated under reduced pressure and the resulting residue was purified by column chromatography to afford tert-butyl (2-((R)-5-((tert-butoxycarbonyl)amino)hexyl)pyridin-4-yl)(1-(tert-butyl)-3-((1S,3R)-3-((tert-butyldimethylsilyl)oxy)cyclopentyl)-1H-pyrazol-5-yl)carbamate (220 mg, 67.6% yield) as a yellow oil. LCMS: 714.6 [M+H]⁺.

Step 4: Preparation of tert-butyl (2-((R)-5-((tert-butoxycarbonyl)amino)hexyl)pyridin-4-yl)(1-(tert-butyl)-3-((1S,3R)-3-hydroxycyclopentyl)-1H-pyrazol-5-yl)carbamate

The solution of tert-butyl (2-((R)-5-((tert-butoxycarbonyl)amino)hexyl)pyridin-4-yl)(1-(tert-butyl)-3-((1S,3R)-3-((tert-butyldimethylsilyl)oxy)cyclopentyl)-1H-pyrazol-5-yl)carbamate (200 mg, 0.28 mmol, 1 eq) and TBAF (366.16 mg, 1.40 mmol, 5 eq) in THF (5 mL) was stirred at 40° C. for 2 hours under N₂ atmosphere. The mixture was then cooled to room temperature, diluted with H₂O (30 mL), and extracted with ethyl acetate (30 mL×2). The combined organic layers were washed with brine (30 mL), dried over Na₂SO₄, filtered and the filtrate was concentrated in vacuo to afford tert-butyl (2-((R)-5-((tert-butoxycarbonyl)amino)hexyl)pyridin-4-yl)(1-(tert-butyl)-3-((1S,3R)-3-hydroxycyclopentyl)-1H-pyrazol-5-yl)carbamate (180 mg, 96.4% yield) as a brown oil. LCMS: 600.4 [M+H]⁺.

Step 5: Preparation of tert-butyl (2-((R)-5-((tert-butoxycarbonyl)amino)hexyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((R)-5-((tert-butoxycarbonyl)amino)hexyl)pyridin-4-yl)(1-(tert-butyl)-3-((1S,3R)-3-hydroxycyclopentyl)-1H-pyrazol-5-yl)carbamate (180 mg, 0.30 mmol, 1 eq) in THF (20 mL) was added DMAP (36.66 mg, 0.30 mmol, 1 eq) and pyridine (237.38 mg, 3.00 mmol, 10 eq), followed by the addition of a solution of (4-nitrophenyl) carbonochloridate (362.93 mg, 1.80 mmol, 6 eq) in THF (5 mL) under N₂ atmosphere. After the completion of the addition, the mixture was stirred at 20° C. for 12 hours under N₂ atmosphere. The reaction mixture was then diluted with H₂O (30 mL) and extracted with ethyl acetate (30 mL×3). The combined organic layers were washed with brine (30 mL), dried over Na₂SO₄, filtered and the filtrate was concentrated under reduced pressure. The resulting residue was purified by column chromatography to afford tert-butyl (2-((R)-5-((tert-butoxycarbonyl)amino)hexyl)pyridin-4-yl)(1-(tert-butyl)-3-((1S,3R)-3-(((4-nitrophenoxy)carbonyl)oxy)cyclopentyl)-1H-pyrazol-5-yl)carbamate (190 mg, 0.248 mmol, 82.8% yield) as a yellow oil. LCMS: 765.4 [M+H]⁺.

Step 6: Preparation of (1R,3S)-3-(5-((2-((R)-5-aminohexyl)pyridin-4-yl)amino)-1-(tert-butyl)-1H-pyrazol-3-yl)cyclopentyl (4-nitrophenyl) carbonate

The solution of tert-butyl (2-((R)-5-((tert-butoxycarbonyl)amino)hexyl)pyridin-4-yl)(1-(tert-butyl)-3-((1S,3R)-3-(((4-nitrophenoxy)carbonyl)oxy)cyclopentyl)-1H-pyrazol-5-yl)carbamate (160 mg, 0.209 mmol, 1 eq) and TFA (119.25 mg, 1.05 mmol, 5 eq) in DCM (3 mL) was stirred at 40° C. for 1 hour under N₂ atmosphere. After the completion of the reaction, the mixture was cooled to room temperature, and concentrated in vacuo to afford (1R,3S)-3-(5-((2-((R)-5-aminohexyl)pyridin-4-yl)amino)-1-(tert-butyl)-1H-pyrazol-3-yl)cyclopentyl (4-nitrophenyl) carbonate (130 mg, 91.6% yield) as a yellow oil. LCMS: 565.3 [M+H]⁺.

Step 7: Preparation of (1¹S,1³R,9R,Z)-2¹-(tert-butyl)-9-methyl-2¹H-12-oxa-3,10-diaza-4(4,2)-pyridina-2(3,5)-pyrazola-1(1,3)-cyclopentanacyclododecaphan-11-one

To a solution of (1R,3S)-3-(5-((2-((R)-5-aminohexyl)pyridin-4-yl)amino)-1-(tert-butyl)-1H-pyrazol-3-yl)cyclopentyl (4-nitrophenyl) carbonate (120 mg, 0.231 mmol, 1 eq) in THF (150 mL) was added DIPEA (137.33 mg, 1.06 mmol, 5 eq), and the mixture was stirred at 100° C. for 3 hours under N₂ atmosphere. After the completion of the reaction, the mixture was cooled to room temperature and concentrated under reduced pressure. The resulting residue was purified by prep-TLC (Petroleum ether/Ethyl acetate=0/1, TLC: PE/EA=0/1, R_{f}=0.5) to afford (1¹S,1³R,9R,Z)-2¹-(tert-butyl)-9-methyl-2¹H-12-oxa-3,10-diaza-4(4,2)-pyridina-2(3,5)-pyrazola-1(1,3)-cyclopentanacyclododecaphan-11-one (90 mg, 99.5% yield) as a yellow solid. LCMS: 426.3 [M+H]⁺.

Step 8: Preparation of (1¹S,1³R,9R,Z)-9-methyl-2¹H-12-oxa-3,10-diaza-4(4,2)-pyridina-2(3,5)-pyrazola-1(1,3)-cyclopentanacyclododecaphan-11-one

A mixture of (1¹S,1³R,9R,Z)-2¹-(tert-butyl)-9-methyl-2¹H-12-oxa-3,10-diaza-4(4,2)-pyridina-2(3,5)-pyrazola-1(1,3)-cyclopentanacyclododecaphan-11-one (90 mg, 0.212 mmol, 1 eq) in formic acid (5 mL) was stirred at 100° C. for 2 hours under N₂ atmosphere. After the completion of the reaction, the mixture was cooled to room temperature and concentrated under reduced pressure. The resulting residue was purified by prep-HPLC (column: Phenomenex luna C18 150*25 mm*10 um; mobile phase: [water (FA)-ACN]; gradient:8%-38% B over 10 min) to afford (1¹S,1³R,9R,Z)-9-methyl-2¹H-12-oxa-3,10-diaza-4(4,2)-pyridina-2(3,5)-pyrazola-1(1,3)-cyclopentanacyclododecaphan-11-one (31.77 mg, 40.5% yield) as a white solid. LCMS: 370.2 [M+H]⁺; ¹H NMR (400 MHz, DMSO-d₆) 5=8.96 (s, 1H), 8.19 (s, 1H), 8.03-8.01 (d, 1H), 6.79 (s, 1H), 6.64-6.59 (m, 2H), 5.93 (s, 1H), 5.11 (s, 1H), 3.49-3.15 (m, 3H), 2.60-2.50 (m, 2H), 1.84 (s, 1H), 1.74-1.58 (m, 6H), 1.29-1.03 (m, 4H), 1.02-0.97 (m, 3H) ppm.

Example 22: Synthesis of (1¹S,1³R,2⁴Z,5E,9S)-9-methyl-2¹H-12-oxa-3,10-diaza-4(4,2)-pyridina-2(3,5)-pyrazola-1(1,3)-cyclopentanacyclododecaphan-5-en-11-one (compound 125)

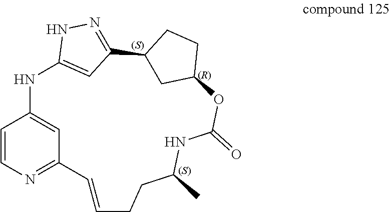

Step 1: Preparation of tert-butyl (S)-(5-hydroxypentan-2-yl)carbamate

To a solution of ethyl (S)-4-((tert-butoxycarbonyl)amino)pentanoate (15.0 g, 61.15 mmol, 1 eq) in THF (150 mL) was added LiBH₄ (3.0 g, 137.72 mmol, 2.25 eq), and the mixture was stirred at 50° C. for 3 hours under N₂ atmosphere. The mixture was then cooled to 0° C. and carefully quenched with saturated aqueous HCl (1M, 100 mL). The mixture was extracted with EtOAc (100 mL×3). The combined organic layers were washed with brine (100 mL×3), dried over anhydrous Na₂SO₄, filtered and the filtrate was concentrated under reduced pressure. The resulting residue was purified by column chromatography to give tert-butyl (S)-(5-hydroxypentan-2-yl)carbamate (12.0 g, 96.5% yield) as a light-yellow oil. ¹H NMR (DMSO-d₆) δ 4.70 (br, 1H), 4.68-4.65 (m, 3H), 2.55-2.28 (br, 1H), 1.63-1.59 (m, 2H), 1.52-1.49 (m, 2H), 1.45 (m, 10H), 1.45-1.13 (m, 3H) ppm.

Step 2: Preparation of tert-butyl (S)-(5-oxopentan-2-yl)carbamate

To a solution of tert-butyl (S)-(5-hydroxypentan-2-yl)carbamate (4.0 g, 19.68 mmol, 1 eq) in DCM (40 mL) was added saturated aqueous NaClO (2.93 g, 39.36 mmol, 2.43 mL, 2 eq), saturated aqueous NaHCO₃(28 mL, 36.00 eq) and TEMPO (309.43 mg, 1.97 mmol, 0.1 eq), and the mixture was stirred at 20° C. for 2 hours under N₂ atmosphere. The reaction mixture was then extracted with EtOAc (100 mL×3). The combined organic layers were washed with brine (100 mL), dried over Na₂SO₄, filtered and the filtrate was concentrated under reduced pressure. The resulting crude was purified by column chromatography to give tert-butyl (S)-(5-oxopentan-2-yl)carbamate (1.1 g, 25.0% yield) as a colorless oil.

Step 3: Preparation of tert-butyl (S)-hex-5-yn-2-ylcarbamate

The mixture of tert-butyl (S)-(5-oxopentan-2-yl)carbamate (363.64 mg, 1.81 mmol, 1 eq), K₂CO₃ (749.13 mg, 5.42 mmol, 3 eq) and dimethyl (1-diazo-2-oxopropyl)phosphonate (694.21 mg, 3.61 mmol, 2 eq) in MeOH (10 mL) was stirred at 20° C. for 12 hours under N₂ atmosphere. After the completion of the reaction, the mixture was diluted with water (50 mL) and extracted with EtOAc (50 mL×3). The combined organic layers were washed with brine (50 mL), dried over anhydrous Na₂SO₄, filtered and the filtrate was concentrated under reduced pressure. The resulting residue was purified by column chromatography to give tert-butyl (S)-hex-5-yn-2-ylcarbamate (170 mg, 44.4% yield) as a colorless oil. ¹H NMR (DMSO-d₆) δ: 4.37 (br, 1H), 3.68-3.65 (m, 1H), 1.96-1.95 (m, 1H), 1.63-1.52 (m, 2H), 1.45 (m, 9H), 1.23-1.18 (m, 3H) ppm.

Step 4: Preparation of tert-butyl (S,E)-(6-(4,4,5,5-tetramethyl-1,3,2-dioxaborolan-2-yl)hex-5-en-2-yl)carbamate

To a mixture of 4,4,5,5-tetramethyl-2-(4,4,5,5-tetramethyl-1,3,2-dioxaborolan-2-yl)-1,3,2-dioxaborolane (675.80 mg, 2.66 mmol, 1.5 eq), NaOMe (19.17 mg, 0.35 mmol, 0.2 eq) and Cu (56.37 mg, 0.887 mmol, 0.5 eq) in EtOH (5 mL) was added tert-butyl (S)-hex-5-yn-2-ylcarbamate (350 mg, 1.77 mmol, 1 eq) under N₂, and the mixture was stirred at 20° C. for 12 hours under N₂ atmosphere. The mixture was filtered through a short pad of celite and rinsed with ethyl acetate (50 mL). The combined filtrate was evaporated to afford tert-butyl (S,E)-(6-(4,4,5,5-tetramethyl-1,3,2-dioxaborolan-2-yl)hex-5-en-2-yl)carbamate (400 mg, 41.6% yield) as a colorless oil, which was used in the next step without further purification. LCMS: 610.4 [M+H]⁺.

Step 5: Preparation of tert-butyl (S,E)-(6-(4-chloropyridin-2-yl)hex-5-en-2-yl)carbamate

A mixture of tert-butyl (S,E)-(6-(4,4,5,5-tetramethyl-1,3,2-dioxaborolan-2-yl)hex-5-en-2-yl)carbamate (100 mg, 0.31 mmol, 1 eq), Pd(dppf)Cl₂ (22.50 mg, 0.03 mmol, 0.1 eq), K₂CO₃ (127.48 mg, 0.92 mmol, 3 eq) and 2-bromo-4-chloro-pyridine (88.75 mg, 0.46 mmol, 1.5 eq) in dioxane (1 mL) and H₂O (0.1 mL) was degassed and backfilled with N₂ for 3 times, and stirred at 80° C. for 2 hours under N₂ atmosphere. The mixture was then cooled to room temperature and diluted with EtOAc (100 mL) and water (100 mL). The organic layer was collected and the aqueous phase was extracted with EtOAc (100 mL×3). The combined organic layers were washed with brine (100 mL), dried over Na₂SO₄, filtered and the filtrate was concentrated under reduced pressure. The resulting residue was purified by Prep-TLC (Petroleum ether: Ethyl acetate=3:1) to give tert-butyl (S,E)-(6-(4-chloropyridin-2-yl)hex-5-en-2-yl)carbamate (25 mg, 25.6% yield) as a colorless oil. LCMS: 311.2 [M+H]⁺.

Step 6: Preparation of tert-butyl ((S,E)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-en-2-yl)carbamate

A mixture of tert-butyl (S,E)-(6-(4-chloropyridin-2-yl)hex-5-en-2-yl)carbamate (90 mg, 0.29 mmol, 1 eq), 1-(tert-butyl)-3-((1S,3R)-3-((tert-butyldimethylsilyl)oxy)cyclopentyl)-1H-pyrazol-5-amine (100.00 mg, 0.29 mmol, 1.02 eq), Pd₂(dba)₃ (20.00 mg, 0.021 mol, 0.07 eq), Xantphos (20.00 mg, 0.03 mmol, 0.01 eq) and Cs₂CO₃ (200.00 mg, 0.61 mmol 2.12 eq) in anhydrous dioxane (3 mL) was stirred for 12 hours at 90° C. under N₂ atmosphere. The mixture was then cooled to room temperature and diluted with EtOAc (200 mL) and water (100 mL). The organic layer was collected and washed with brine (100 mL). The organic phase was dried over anhydrous Na₂SO₄, filtered and the filtrate was concentrated. The resulting crude was purified by Prep-TLC (Petroleum ether: EtOAc=1:2) to give tert-butyl ((S,E)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-en-2-yl)carbamate (164 mg, 83.3% yield) as a yellow solid. LCMS: 612.6 [M+H]⁺.

Step 7: Preparation of tert-butyl (2-((S,E)-5-((tert-butoxycarbonyl)amino)hex-1-en-1-yl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S,E)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-en-2-yl)carbamate (164 mg, 0.29 mmol, 1 eq) in anhydrous DCM (5 mL) was added Boc₂O (90 mg, 0.41 mmol, 1.54 eq) and DMAP (50 mg, 0.41 mmol, 1.53 eq) at 20° C., and the mixture was stirred for 2 hour under N₂ atmosphere. The mixture was concentrated, and the remaining residue was diluted with EtOAc (200 mL) and water (100 mL). The organic layer was collected and washed with brine (100 mL). The organic phase was dried over anhydrous Na₂SO₄, filtered and the filtrate was concentrated to afford tert-butyl (2-((S,E)-5-((tert-butoxycarbonyl)amino)hex-1-en-1-yl)pyridin-4-yl)(1-(tert-butyl)-3-((1S,3R)-3-((tert-butyldimethylsilyl)oxy)cyclopentyl)-1H-pyrazol-5-yl)carbamate (185 mg, 96.9% yield), which was used in next step directly without further purification. LCMS: 712.7 [M+H]⁺.

Step 8: Preparation of tert-butyl (2-((S,E)-5-((tert-butoxycarbonyl)amino)hex-1-en-1-yl)pyridin-4-yl)(1-(tert-butyl)-3-((1S,3R)-3-hydroxycyclopentyl)-1H-pyrazol-5-yl)carbamate

A mixture of tert-butyl (2-((S,E)-5-((tert-butoxycarbonyl)amino)hex-1-en-1-yl)pyridin-4-yl)(1-(tert-butyl)-3-((1S,3R)-3-((tert-butyldimethylsilyl)oxy)cyclopentyl)-1H-pyrazol-5-yl)carbamate (175 mg, 0.25 mmol, 1 eq) in TBAF (10 mL) was stirred for 2 hour at 20° C. under N₂ atmosphere. The mixture was then diluted with 100 mL of water, extracted with EtOAc (50 mL×3). The combined organic layers were washed with brine (50 mL), dried over anhydrous Na₂SO₄, filtered and the filtrate was concentrated. The title compound tert-butyl (2-((S,E)-5-((tert-butoxycarbonyl)amino)hex-1-en-1-yl)pyridin-4-yl)(1-(tert-butyl)-3-((1S,3R)-3-hydroxycyclopentyl)-1H-pyrazol-5-yl)carbamate (145 mg, 0.24 mmol) was used in next step directly without further purification. LCMS: 598.6 [M+H]⁺.

Step 9: Preparation of tert-butyl (2-((S,E)-5-((tert-butoxycarbonyl)amino)hex-1-en-1-yl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((S,E)-5-((tert-butoxycarbonyl)amino)hex-1-en-1-yl)pyridin-4-yl)(1-(tert-butyl)-3-((1S,3R)-3-hydroxycyclopentyl)-1H-pyrazol-5-yl)carbamate (100 mg, 0.16 mmol, 1 eq) in anhydrous DCM (2 mL) was added DMAP (50 mg, 0.4 mmol, 2.45 eq) and pyridine (40 mg, 0.51 mmol, 3.02 eq), followed by the addition of a solution of (4-nitrophenyl) carbonochloridate (140 mg, 0.69 mmol, 4.15 eq) in DCM (3 mL) at 0° C. under N₂ atmosphere. After the completion of the addition, the mixture was warmed to 20° C. and stirred for 0.5 hour at that temperature under N₂ atmosphere. The reaction was then quenched with water (10 mL) and extracted with EtOAc (20 mL×3). The combined organic layers were washed with brine (30 mL), dried over anhydrous Na₂SO₄, filtered and the filtrate was concentrated. The desired tert-butyl (2-((S,E)-5-((tert-butoxycarbonyl)amino)hex-1-en-1-yl)pyridin-4-yl)(1-(tert-butyl)-3-((1S,3R)-3-(((4-nitrophenoxy)carbonyl)oxy)cyclopentyl)-1H-pyrazol-5-yl)carbamate (130 mg, 78.4% yield) was used in next step directly without further purification. LCMS: 763.6 [M+H]⁺.

Step 10: Preparation of (1R,3S)-3-(5-((2-((S,E)-5-aminohex-1-en-1-yl)pyridin-4-yl)amino)-1-(tert-butyl)-1H-pyrazol-3-yl)cyclopentyl (4-nitrophenyl) carbonate

The solution of tert-butyl (2-((S,E)-5-((tert-butoxycarbonyl)amino)hex-1-en-1-yl)pyridin-4-yl)(1-(tert-butyl)-3-((1S,3R)-3-(((4-nitrophenoxy)carbonyl)oxy)cyclopentyl)-1H-pyrazol-5-yl)carbamate (125 mg, 0.16 mmol, 1 eq) and TFA (3.07 g, 26.92 mmol, 2 mL, 164.32 eq) in anhydrous DCM (3 mL) was stirred at 20° C. for 2 hours under N₂ atmosphere. The mixture was concentrated to give 92 mg of the title compound (1R,3S)-3-(5-((2-((S,E)-5-aminohex-1-en-1-yl)pyridin-4-yl)amino)-1-(tert-butyl)-1H-pyrazol-3-yl)cyclopentyl (4-nitrophenyl) carbonate, which was used in next step directly without further purification. LCMS: 563.5 [M+H]⁺.

Step 11: Preparation of (1¹S,1³R,2⁴Z,5E,9S)-2¹-(tert-butyl)-9-methyl-2¹H-12-oxa-3,10-diaza-4(4,2)-pyridina-2(3,5)-pyrazola-1(1,3)-cyclopentanacyclododecaphan-5-en-11-one

A mixture of (1R,3S)-3-(5-((2-((S,E)-5-aminohex-1-en-1-yl)pyridin-4-yl)amino)-1-(tert-butyl)-1H-pyrazol-3-yl)cyclopentyl (4-nitrophenyl) carbonate (92 mg, 0.16 mmol, 1 eq) and DIEA (211.32 mg, 1.64 mmol, 10 eq) in anhydrous THF (100 mL) was stirred for 2 hours at 60° C. under N₂ atmosphere. After the completion of the reaction, the mixture was cooled to room temperature. The mixture was concentrated, and the remaining residue was dissolved in EtOAc (200 mL). The EtOAc phase was washed with saturated aqueous NaHCO₃(100 mL) and brine (100 mL). The organic layer was dried over anhydrous Na₂SO₄, filtered and the filtrate was concentrated to give (1¹S,1³R,2⁴Z,5E,9S)-2¹-(tert-butyl)-9-methyl-2¹H-12-oxa-3,10-diaza-4(4,2)-pyridina-2(3,5)-pyrazola-1(1,3)-cyclopentanacyclododecaphan-5-en-11-one (65 mg, 0.15 mmol) as a yellow oil, which was used in next step without further purification. LCMS: 424.3 [M+H]⁺.

Step 12: Preparation of (1¹S,1³R,2⁴Z,5E,9S)-9-methyl-2¹H-12-oxa-3,10-diaza-4(4,2)-pyridina-2(3,5)-pyrazola-1(1,3)-cyclopentanacyclododecaphan-5-en-11-one

A mixture of (1¹S,1³R,2⁴Z,5E,9S)-2¹-(tert-butyl)-9-methyl-2¹H-12-oxa-3,10-diaza-4(4,2)-pyridina-2(3,5)-pyrazola-1(1,3)-cyclopentanacyclododecaphan-5-en-11-one (65 mg, 0.15 mmol, 1 eq) in formic acid (5 mL) was stirred for 1 hour at 100° C. under N₂ atmosphere. After the completion of the reaction, the mixture was cooled to room temperature, concentrated and the resulting residue was purified by prep-HPLC (column: Waters Xbridge 150*25 mm*Sum; mobile phase: [water (ammonia hydroxide v/v)-ACN]; gradient: 11%-41% B over 10 min). The desired (1¹S,1³R,2⁴Z,5E,9S)-9-methyl-2¹H-12-oxa-3,10-diaza-4(4,2)-pyridina-2(3,5)-pyrazola-1(1,3)-cyclopentanacyclododecaphan-5-en-11-one (5.71 mg, 9.93% yield, 98.1%) was obtained as a white solid. LCMS: 368.2 [M+H]⁺; 1H NMR (DMSO-d₆) 5=8.93 (s, 1H), 8.53 (s, 1H), 8.10-8.04 (m, 2H), 7.24 (s, 1H), 6.70-6.63 (m, 1H), 6.59-6.56 (m, 2H), 6.09 (m, 1H), 5.14 (s, 1H), 4.85-4.75 (m, 1H), 4.03-3.94 (m, 1H), 3.90-3.84 (m, 1H), 3.71-3.62 (m, 1H), 3.71-3.62 (m, 1H), 2.30-2.25 (m, 1H), 2.23-2.19 (m, 1H), 2.15-2.01 (m, 1H), 1.83-1.79 (m, 3H), 1.51-1.46 (s, 1H), 1.10-1.03 (m, 3H) ppm.

Example 23: synthesis of (1¹S,1³R,2⁴Z,5E,9R)-9-methyl-2¹H-12-oxa-3,10-diaza-4(4,2)-pyridina-2(3,5)-pyrazola-1(1,3)-cyclopentanacyclododecaphan-5-en-11-one (compound 126)

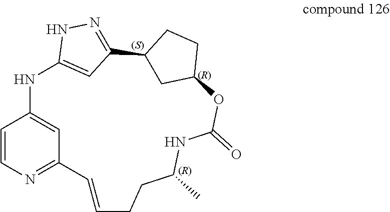

Step 1: Preparation of tert-butyl (R)-(1-oxopropan-2-yl)carbamate

To a solution of tert-butyl (R)-(1-hydroxypropan-2-yl)carbamate (25 g, 142.67 mmol, 1 eq) in DCM (300 mL) was added DMP (66.57 g, 156.94 mmol, 1.1 eq) in portions at 0° C. under N₂ atmosphere. After the completion of the addition, the mixture was warmed to 25° C. and stirred for an additional 0.5 hour at that temperature under N₂ atmosphere. The reaction was then quenched with saturated aqueous NaHCO₃(150 mL), followed by addition of water (500 mL). The mixture was extracted with EtOAc (500 mL×3), and the combined organic layers were washed with brine (500 mL), dried over Na₂SO₄, filtered, and the filtrate was concentrated. The resulting residue was purified by column chromatography to afford tert-butyl (R)-(1-oxopropan-2-yl)carbamate (19 g, 76.9% yield) as a colorless oil. ¹H NMR (400 MHz, DMSO-d₆) δ 9.56 (s, 1H), 5.09 (s, 1H), 4.23 (s, 1H), 1.46 (s, 9H), 1.35 (d, J=7.2 Hz, 3H) ppm.

Step 2: Preparation of methyl (R,E)-4-((tert-butoxycarbonyl)amino)pent-2-enoate

The mixture of tert-butyl (R)-(1-oxopropan-2-yl)carbamate (19 g, 109.69 mmol, 1 eq) and methyl 2-(triphenyl-phosphanylidene)acetate (44.01 g, 131.63 mmol, 1.2 eq) in THF (50 mL) was stirred at 12° C. for 20 hours under N₂ atmosphere. After the starting material was consumed, the reaction mixture was quenched with water (200 mL) and extracted with EtOAc (100 mL×3). The combined organic phases were washed with brine (50 mL), dried over Na₂SO₄, filtered, and the filtrate was concentrated. The resulting residue was purified by column chromatography to give methyl (R,E)-4-((tert-butoxycarbonyl)amino)pent-2-enoate (23.84 g, 94.8% yield) as a colorless oil.

Step 3 Preparation of methyl (R)-4-((tert-butoxycarbonyl)amino)pentanoate

The mixture of methyl (R,E)-4-((tert-butoxycarbonyl)amino)pent-2-enoate (23.84 g, 103.98 mmol, 1 eq) and Pd/C (5.23 g, 10% purity) in MeOH (200 mL) was degassed and backfilled with H₂ for three times, and then stirred under H₂ (50 psi) at 25° C. for 12 hours. After the reaction was completed, the mixture was filtered through a short pad of celite, rinsed with MeOH (50 mL), and the combined filtrate was concentrated. The resulting residue was purified by column chromatography to afford methyl (R)-4-((tert-butoxycarbonyl)amino)pentanoate (14.5 g, 60.3% yield) as a colorless oil. ¹H NMR (400 MHz, DMSO-d₆) δ 6.98-6.73 (m, 1H), 5.94-5.73 (m, 1H), 4.74-5.63 (m, 1H), 4.44-4.23 (m, 1H), 4.19-4.06 (m, 1H), 3.73-3.66 (m, 3H), 2.13-1.96 (m, 1H), 1.43-1.36 (m, 10H), 1.33-1.16 (m, 3H) ppm.

Step 4: Preparation of tert-butyl (R)-(5-hydroxypentan-2-yl)carbamate

To the solution of methyl (R)-4-((tert-butoxycarbonyl)amino)pentanoate (14.5 g, 62.69 mmol, 1 eq) in THF (250 mL), LiBH₄ (5.01 g, 230.08 mmol, 3.67 eq) was added in portions at 0° C. under N₂ atmosphere. After the completion of the addition, the mixture was warmed to 50° C. and stirred for 3 hours under N₂ atmosphere. The mixture was then cooled to 0° C., quenched with saturated aqueous NH₄Cl (100 mL) and extracted with EtOAc (100 mL×3). The combined organic layers were washed with brine (200 mL×3), dried over Na₂SO₄, filtered, and the filtrate was concentrated under reduced pressure. The resulting residue was purified by column chromatography to afford tert-butyl (R)-(5-hydroxypentan-2-yl)carbamate (15.5 g, 62.69 mmol) as a white solid. ¹H NMR (400 MHz, DMSO-d₆) δ 4.57 (s, 1H), 3.58-3.55 (m, 2H), 2.91 (s, 1H), 1.56-1.51 (m, 2H), 1.50-1.45 (m, 2H), 1.43-1.41 (m, 9H), 1.37-1.05 (m, 3H) ppm.

Step 5: Preparation of tert-butyl (R)-(5-oxopentan-2-yl)carbamate

To a solution of tert-butyl (R)-(5-hydroxypentan-2-yl)carbamate (10 g, 49.19 mmol, 1 eq) in DCM (10 mL) was added TEMPO (773.59 mg, 4.92 mmol, 0.1 eq), followed by the addition of the mixture of aqueous NaClO (7.32 g, 98.34 mmol, 6.07 mL, 2.00 eq) and aqueous NaHCO₃(148.76 g, 1.77 mol, 68.9 mL, 36.00 eq). After the completion of the addition, the mixture was stirred at 20° C. for 12 hours. The reaction mixture was then concentrated under reduced pressure to remove DCM, and the resulting residue was extracted with EtOAc (100 mL×3). The combined organic layers were washed with brine (100 mL), dried over Na₂SO₄, filtered and the filtrate was concentrated under reduced pressure. The resulting crude was purified by column chromatography to afford tert-butyl (R)-(5-oxopentan-2-yl)carbamate (4.35 g, 44.0% yield) as a yellow oil. ¹H NMR (400 MHz, DMSO-d₆) δ 5.45 (s, 1H), 3.81-3.65 (m, 1H), 2.29 (s, 1H), 2.00-1.86 (m, 2H), 1.73 (s, 1H), 1.64-1.49 (m, 9H), 1.45-1.36 (m, 2H), 1.12-1.11 (m, 2H) ppm.

Step 6: Preparation of tert-butyl (R)-hex-5-yn-2-ylcarbamate

The mixture of tert-butyl (R)-(5-oxopentan-2-yl)carbamate (4 g, 19.87 mmol, 1 eq), K₂CO₃ (8.24 g, 59.62 mmol, 3 eq) and 1-diazo-1-dimethoxyphosphoryl-propan-2-one (5.73 g, 29.81 mmol, 1.5 eq) in MeOH (60 mL) was stirred at 25° C. for 12 hours under N₂ atmosphere. The reaction mixture was then concentrated under reduced pressure and the remaining residue was diluted with water (60 mL) and extracted with EtOAc (60 mL×3). The combined organic layers were washed with brine (50 mL), dried over anhydrous Na₂SO₄, filtered and the filtrate was concentrated under reduced pressure. The resulting residue was purified by column chromatography to afford tert-butyl (R)-hex-5-yn-2-ylcarbamate (3.0 g, 76.5% yield) as a white solid. ¹H NMR (400 MHz, DMSO-d₆) δ 4.37 (s, 1H), 3.75-3.71 (m, 1H), 2.25-2.21 (m, 2H), 1.95 (s, 1H), 1.66-1.65 (m, 2H), 1.48-1.44 (m, 9H), 1.15-1.13 (m, 3H) ppm.

Step 7: Preparation of tert-butyl (R,E)-(6-(4,4,5,5-tetramethyl-1,3,2-dioxaborolan-2-yl)hex-5-en-2-yl)carbamate

To a mixture of 4,4,5,5-tetramethyl-2-(4,4,5,5-tetramethyl-1,3,2-dioxaborolan-2-yl)-1,3,2-dioxaborolane (424.79 mg, 1.67 mmol, 1.1 eq), NaOMe (16.43 mg, 0.304 mmol, 0.2 eq) and Cu (9.66 mg, 0.152 mmol, 0.1 eq) in EtOH (4 mL) was added tert-butyl (R)-hex-5-yn-2-ylcarbamate (0.3 g, 1.52 mmol, 1 eq) under N₂, and the mixture was stirred at 20° C. for 12 hours under N₂ atmosphere. The mixture was filtered through a short pad of celite and rinsed with ethyl acetate (50 mL). The combined filtrate was evaporated to afford 340 mg of tert-butyl (R,E)-(6-(4,4,5,5-tetramethyl-1,3,2-dioxaborolan-2-yl)hex-5-en-2-yl)carbamate as a yellow oil, which was used in the next step without further purification.

Step 8: Preparation of tert-butyl N-[(E,1R)-5-(4-chloro-2-pyridyl)-1-methyl-pent-4-enyl]carbamate

The mixture of tert-butyl (R,E)-(6-(4,4,5,5-tetramethyl-1,3,2-dioxaborolan-2-yl)hex-5-en-2-yl)carbamate (0.34 g, 1.05 mmol, 1 eq) and 2-bromo-4-chloro-pyridine (402.34 mg, 2.09 mmol, 2 eq) in dioxane (10 mL)-water (1 mL) was degassed and backfilled with N₂ for 3 times, followed by the addition of Pd(dppf)Cl₂ (76.49 mg, 0.104 mmol, 0.1 eq) and K₂CO₃ (433.42 mg, 3.14 mmol, 3 eq) under N₂ atmosphere. The mixture was then stirred at 80° C. for 1.5 hours under N₂ atmosphere. After the reaction was completed, the mixture was cooled to room temperature, and diluted with EtOAc (100 mL) and water (100 mL). The organic layer was collected, and the aqueous phase was extracted with EtOAc (100 mL×3). The combined organic layers were washed with brine (100 mL), dried over Na₂SO₄, filtered and the filtrate was concentrated under reduced pressure. The resulting residue was purified by column chromatography to afford tert-butyl (R,E)-(6-(4-chloropyridin-2-yl)hex-5-en-2-yl)carbamate (0.24 g, 73.9% yield) as a light-yellow oil. LCMS: 311.2 [M+H]⁺.

Step 9: Preparation of tert-butyl ((R,E)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-en-2-yl)carbamate

The mixture of tert-butyl (R,E)-(6-(4-chloropyridin-2-yl)hex-5-en-2-yl)carbamate (0.15 g, 0.482 mmol, 1 eq), 1-(tert-butyl)-3-((1S,3R)-3-((tert-butyldimethylsilyl)oxy)cyclopentyl)-1H-pyrazol-5-amine (162.91 mg, 0.482 mmol, 1 eq), Pd₂(dba)₃ (44.19 mg, 0.048 mmol, 0.1 eq), Cs₂CO₃ (471.72 mg, 1.45 mmol, 3 eq) and Xantphos (46.01 mg, 0.096 mmol, 0.2 eq) in dioxane (3 mL) was stirred at 90° C. for 12 hours under N₂. After the completion of the reaction, the mixture was cooled to room temperature and diluted with EtOAc (50 mL) and water (50 mL). The organic layer was collected, and the aqueous phase was extracted with EtOAc (30 mL×3). The combined organic layers were washed with brine (50 mL), dried over Na₂SO₄, filtered, and the filtrate was concentrated under reduced pressure. The resulting residue was purified by prep-TLC (PE/EA=1/2) to afford tert-butyl ((R,E)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-en-2-yl)carbamate (0.24 g, 81.3% yield) as yellow oil.

Step 10: Preparation of tert-butyl (2-((R,E)-5-((tert-butoxycarbonyl)amino)hex-1-en-1-yl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((R,E)-6-(4-((1-(tert-butyl)-3-((1S,3R)-3-((tert-butyldimethylsilyl)oxy)cyclopentyl)-1H-pyrazol-5-yl)amino)pyridin-2-yl)hex-5-en-2-yl)carbamate (0.24 g, 0.392 mmol, 1 eq) in DCM (10 mL) was added Boc₂O (102.72 mg, 0.470 mmol, 1.2 eq) and DMAP (95.83 mg, 0.78 mmol, 2 eq) at 0° C., and the mixture was then warmed to 25° C. and stirred for 4 hours under N₂ atmosphere. After the completion of the reaction, the volatiles were removed under reduced pressure. The resulting residue was purified by prep-TLC (PE/EA=3/1) to afford tert-butyl (2-((R,E)-5-((tert-butoxycarbonyl)amino)hex-1-en-1-yl)pyridin-4-yl)(1-(tert-butyl)-3-((1S,3R)-3-((tert-butyldimethylsilyl)oxy)cyclopentyl)-1H-pyrazol-5-yl)carbamate (0.19 g, 0.266 mmol, 68.0% yield) as a light-yellow oil. LCMS: 712.4 [M+H]⁺.

Step 11: Preparation of tert-butyl (2-((R,E)-5-((tert-butoxycarbonyl)amino)hex-1-en-1-yl)pyridin-4-yl)(1-(tert-butyl)-3-((1S,3R)-3-hydroxycyclopentyl)-1H-pyrazol-5-yl)carbamate

The mixture of solution of tert-butyl (2-((R,E)-5-((tert-butoxycarbonyl)amino)hex-1-en-1-yl)pyridin-4-yl)(1-(tert-butyl)-3-((1S,3R)-3-((tert-butyldimethylsilyl)oxy)cyclopentyl)-1H-pyrazol-5-yl)carbamate (0.19 g, 0.266 mmol, 1 eq) and TBAF (1 M, 0.5 mL, 1.87 eq) in THF (0.5 mL) was stirred at 25° C. for 12 hours under N₂ atmosphere. The reaction mixture was diluted with EtOAc (50 mL), washed with water (50 mL×3), and evaporated to dryness under reduced pressure. The resulting residue was purified by prep-TLC (PE/EA=1/1) to afford tert-butyl (2-((R,E)-5-((tert-butoxycarbonyl)amino)hex-1-en-1-yl)pyridin-4-yl)(1-(tert-butyl)-3-((1S,3R)-3-hydroxycyclopentyl)-1H-pyrazol-5-yl)carbamate (0.12 g, 75.2% yield) as a light-yellow oil. LCMS: 598.3 [M+H]⁺.

Step 12: Preparation of tert-butyl (2-((R,E)-5-((tert-butoxycarbonyl)amino)hex-1-en-1-yl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((R,E)-5-((tert-butoxycarbonyl)amino)hex-1-en-1-yl)pyridin-4-yl)(1-(tert-butyl)-3-((1S,3R)-3-hydroxycyclopentyl)-1H-pyrazol-5-yl)carbamate (0.12 g, 0.20 mmol, 1 eq) in THF (1 mL) was added pyridine (95.27 mg, 1.20 mmol, 6 eq) and DMAP (12.26 mg, 0.10 mmol, 0.5 eq), followed by the addition of a solution of (4-nitrophenyl) carbonochloridate (121.39 mg, 0.60 mmol, 3 eq) in THF (1 mL) at 0° C., and the mixture was then warmed to 25° C. and stirred for 12 hours under N₂ atmosphere. After the completion of the reaction, the reaction mixture was diluted with EtOAc (100 mL) and washed with brine (20 mL×3). The organic layer was dried over Na₂SO₄, filtered and the filtrate was concentrated under reduced pressure. The resulting residue was purified by prep-HPLC (FA as additive) to afford tert-butyl (2-((R,E)-5-((tert-butoxycarbonyl)amino)hex-1-en-1-yl)pyridin-4-yl)(1-(tert-butyl)-3-((1S,3R)-3-(((4-nitrophenoxy)carbonyl)oxy)cyclopentyl)-1H-pyrazol-5-yl)carbamate (50 mg, 0.065 mmol, 32.6% yield) as a light-yellow oil. LCMS: 763.6 [M+H]⁺.

Step 13: Preparation of (1R,3S)-3-(5-((2-((R,E)-5-aminohex-1-en-1-yl)pyridin-4-yl)amino)-1-(tert-butyl)-1H-pyrazol-3-yl)cyclopentyl (4-nitrophenyl) carbonate

The mixture of tert-butyl (2-((R,E)-5-((tert-butoxycarbonyl)amino)hex-1-en-1-yl)pyridin-4-yl)(1-(tert-butyl)-3-((1S,3R)-3-(((4-nitrophenoxy)carbonyl)oxy)cyclopentyl)-1H-pyrazol-5-yl)carbamate (0.04 g, 0.052 mmol, 1 eq) and TFA (1.54 g, 13.46 mmol, 1 mL, 256.75 eq) in DCM (1 mL) was stirred at 35° C. for 0.5 hour under N₂ atmosphere. After the completion of the reaction, the mixture was evaporated to dryness under reduced pressure to afford (1R,3S)-3-(5-((2-((R,E)-5-aminohex-1-en-1-yl)pyridin-4-yl)amino)-1-(tert-butyl)-1H-pyrazol-3-yl)cyclopentyl (4-nitrophenyl) carbonate (0.035 g, 0.051 mmol) as a light-yellow oil, which was used in the next step without further purification. LCMS: 563.2 [M+H]⁺.

Step 14: Preparation of (1¹S,1³R,2⁴Z,5E,9R)-2¹-(tert-butyl)-9-methyl-2¹H-12-oxa-3,10-diaza-4(4,2)-pyridina-2(3,5)-pyrazola-1(1,3)-cyclopentanacyclododecaphan-5-en-11-one

To a solution of (1R,3S)-3-(5-((2-((R,E)-5-aminohex-1-en-1-yl)pyridin-4-yl)amino)-1-(tert-butyl)-1H-pyrazol-3-yl)cyclopentyl (4-nitrophenyl) carbonate (0.035 g, 0.051 mmol, 1 eq) in THF (35 mL) was added DIEA (33.42 mg, 0.258 mmol, 5 eq), and the mixture was stirred at 70° C. for 12 hours under N₂. After the completion of the reaction, the mixture was cooled to room temperature and evaporated to dryness under reduced pressure. The resulting residue was purified by prep-TLC (EA) to afford (1¹S,1³R,2⁴Z,5E,9R)-2¹-(tert-butyl)-9-methyl-2¹H-12-oxa-3,10-diaza-4(4,2)-pyridina-2(3,5)-pyrazola-1(1,3)-cyclopentanacyclododecaphan-5-en-11-one (0.027 g, 98.6% yield) as a yellow oil. LCMS: 424.2 [M+H]⁺.

Step 15: Preparation of (1¹S,1³R,2⁴Z,5E,9R)-9-methyl-2¹H-12-oxa-3,10-diaza-4(4,2)-pyridina-2(3,5)-pyrazola-1(1,3)-cyclopentanacyclododecaphan-5-en-11-one

The solution of (1¹S,1³R,2⁴Z,5E,9R)-2¹-(tert-butyl)-9-methyl-2¹H-12-oxa-3,10-diaza-4(4,2)-pyridina-2(3,5)-pyrazola-1(1,3)-cyclopentanacyclododecaphan-5-en-11-one (0.027 g, 0.063 mmol, 1 eq) in formic acid (1 mL) was stirred at 100° C. for 0.5 hour under N₂ atmosphere. After the completion of the reaction, the mixture was cooled to room temperature and evaporated to dryness under reduced pressure. The resulting residue was purified by prep-HPLC (column: Phenomenex luna C18 150*25 mm*10 um; mobile phase: [water(FA)-ACN]; gradient: 5%-35% B over 10 min) to afford (1¹S,1³R,2⁴Z,5E,9R)-9-methyl-2¹H-12-oxa-3,10-diaza-4(4,2)-pyridina-2(3,5)-pyrazola-1(1,3)-cyclopentanacyclododecaphan-5-en-11-one (4.36 mg, 17.68% yield) as a white solid. LCMS: 368.2 [M+H]⁺; ¹H NMR (400 MHz, DMSO-d₆) δ 8.91 (s, 1H), 8.33 (s, 2H), 8.03-8.00 (m, 1H), 8.23 (s, 1H), 7.32 (s, 1H), 6.92-6.57 (m, 1H), 6.43 (s, 1H), 6.38 (s, 1H), 6.29 (s, 1H), 6.14-6.08 (m, 1H) 65.96 (s, 1H), 5.18-4.92 (m, 1H), 3.27 (s, 1H), 2.41-2.34 (m, 1H), 2.19-2.13 (m, 4H), 1.99-1.79 (m, 4H), 1.77-1.43 (m, 2H), 1.05-1.02 (m, 3H) ppm.

Example 24: synthesis of (1¹S,1³R,9S,Z)-9-(trifluoromethyl)-2¹H-5,12-dioxa-3,10-diaza-4(4,2)-pyridina-2(3,5)-pyrazola-1(1,3)-cyclopentanacyclododecaphan-11-one (compound 127)

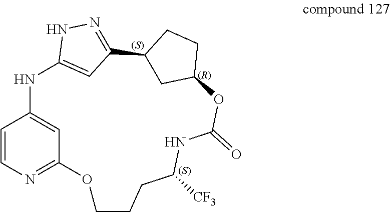

Step 1: Preparation of (S)-4-amino-5,5,5-trifluoropentanoic acid

The solution of (S)-5-(trifluoromethyl)pyrrolidin-2-one (5 g, 32.66 mmol, 1 eq) in HCl (4 M, 220 mL, 26.95 eq) was stirred at 20° C. for 4 hours. After the hydrolysis was completed, the reaction mixture was concentrated under reduced pressure and dried via high vacuum to afford (S)-4-amino-5,5,5-trifluoropentanoic acid (crude, 7 g, 97.1% yield) as a yellow oil, which was used in the next step without further purification. ¹H NMR (400 MHz, MeOD) δ 3.33-3.32 (m, 1H), 2.45-2.26 (m, 2H), 2.25-2.08 (m, 2H) ppm.

Step 2: Preparation of (S)-4-((tert-butoxycarbonyl)amino)-5,5,5-trifluoropentanoic acid

To a solution of (S)-4-amino-5,5,5-trifluoropentanoic acid (5 g, 24.09 mmol, 1 eq) in dioxane (100 mL) and H₂O (50 mL) was added K₂CO₃ (33.29 g, 240.87 mmol, 10 eq) and Boc₂O (7.12 g, 32.64 mmol, 1.35 eq), and the reaction mixture was stirred at 20° C. for 16 hours under N₂ atmosphere. The mixture was then quenched with saturated aqueous NH₄Cl (50 mL) and extracted with EtOAc (50 mL×2). The combined organic layers were washed with brine (50 mL), dried over anhydrous Na₂SO₄, filtered and the filtrate was concentrated in vacuo. The resulting residue was purified by column chromatography to afford (S)-4-((tert-butoxycarbonyl)amino)-5,5,5-trifluoropentanoic acid (700 mg, 10.2% yield) as a white solid. ¹H NMR (400 MHz, DMSO-d₆) δ 7.50-7.48 (d, J=9.2 Hz, 1H), 4.30-4.24 (m, 1H), 4.20-4.12 (m, 1H), 2.41-2.39 (m, 2H), 2.15-1.99 (m, 2H), 1.37 (s, 9H) ppm.

Step 3: Preparation of tert-butyl (S)-(1,1,1-trifluoro-5-hydroxypentan-2-yl)carbamate

To the solution of (S)-4-((tert-butoxycarbonyl)amino)-5,5,5-trifluoropentanoic acid (500 mg, 1.84 mmol, 1 eq) in THF (20 mL) was added BH₃-THF (1 M, 5.53 mL, 3 eq) at 0° C., and the mixture was then warm to 45° C. and stirred for 1 hour under N₂ atmosphere. The mixture was then cooled to room temperature, carefully quenched with 1M HCl (10 mL), and stirred for an additional 0.5 hours under N₂ atmosphere. The mixture was extracted with EtOAc (10 mL×3). The combined organic layers were washed with brine (50 mL), dried over anhydrous Na₂SO₄, filtered and the filtrate was concentrated in vacuo. The resulting residue was purified by column chromatography to afford tert-butyl (S)-(1,1,1-trifluoro-5-hydroxypentan-2-yl)carbamate (400 mg, 73.4% yield) as a white solid.

Step 4: Preparation of tert-butyl (S)-(5-((4-bromopyridin-2-yl)oxy)-1,1,1-trifluoropentan-2-yl)carbamate

To a solution of tert-butyl (S)-(1,1,1-trifluoro-5-hydroxypentan-2-yl)carbamate (400 mg, 1.55 mmol, 1 eq) in THF (10 mL) was added t-BuOK (523.44 mg, 4.66 mmol, 3 eq) and 4-bromo-2-fluoro-pyridine (328.37 mg, 1.87 mmol, 1.2 eq), and the mixture was stirred at 20° C. for 3 hours under N₂ atmosphere. After the starting material was consumed, the reaction mixture was diluted with H₂O (50 mL) and EtOAc (50 mL). The organic layer was collected, and the aqueous phase was extracted with EtOAc (50 mL×3). The combined organic layers were washed with brine (50 mL), dried over anhydrous Na₂SO₄, filtered and the filtrate was concentrated in vacuo. The resulting residue was purified by column chromatography to afford tert-butyl (S)-(5-((4-bromopyridin-2-yl)oxy)-1,1,1-trifluoropentan-2-yl)carbamate (600 mg, 88.7% yield) as a white solid. LCMS: 413.1, 415.1 [M+H]⁺.

Step 5: Preparation of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-1,1,1-trifluoropentan-2-yl)carbamate

The mixture of tert-butyl (S)-(5-((4-bromopyridin-2-yl)oxy)-1,1,1-trifluoropentan-2-yl)carbamate (540 mg, 1.31 mmol, 1 eq), 1-(tert-butyl)-3-((1S,3R)-3-((tert-butyldimethylsilyl)oxy)cyclopentyl)-1H-pyrazol-5-amine (485.25 mg, 1.44 mmol, 1.1 eq), Pd₂(dba)₃ (239.33 mg, 0.26 mmol, 0.2 eq), Cs₂CO₃ (851.55 mg, 2.61 mmol, 2 eq) and Xantphos (124.59 mg, 0.26 mmol, 0.2 eq) in dioxane (10 mL) was stirred at 90° C. for 1 hour under N₂ atmosphere. After the completion of the reaction, the mixture was cooled to room temperature and diluted with EtOAc (50 mL) and water (50 mL). The organic layer was collected, and the aqueous phase was extracted with EtOAc (30 mL×3). The combined organic layers were washed with brine (50 mL), dried over Na₂SO₄, filtered and the filtrate was concentrated under reduced pressure. The resulting residue was purified by column chromatography to afford tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-1,1,1-trifluoropentan-2-yl)carbamate (750 mg, 80.5% yield) as a white solid. LCMS: 670.4 [M+H]⁺.

Step 6: Preparation of tert-butyl (2-(((S)-4-((tert-butoxycarbonyl)amino)-5,5,5-trifluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-1,1,1-trifluoropentan-2-yl)carbamate (700 mg, 1.04 mmol, 1 eq) in DCM (10 mL) was added DMAP (127.66 mg, 1.04 mmol, 1 eq) and Boc₂O (342.08 mg, 1.57 mmol, 1.5 eq), and the mixture was stirred at 25° C. for 4 hours under N₂ atmosphere. After the completion of the reaction, the reaction mixture was diluted with EtOAc (50 mL) and water (50 mL). The organic phase was washed with brine (50 mL), dried over Na₂SO₄, filtered and the filtrate was concentrated under reduced pressure. The resulting residue was purified by column chromatography to afford tert-butyl (2-(((S)-4-((tert-butoxycarbonyl)amino)-5,5,5-trifluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate (750 mg, 87.6% yield) as a white solid. LCMS: 770.5 [M+H]⁺.

Step 7: Preparation of tert-butyl (2-(((S)-4-((tert-butoxycarbonyl)amino)-5,5,5-trifluoropentyl)oxy)pyridin-4-yl)(1-(tert-butyl)-3-((1S,3R)-3-hydroxycyclopentyl)-1H-pyrazol-5-yl)carbamate

The solution of tert-butyl (2-(((S)-4-((tert-butoxycarbonyl)amino)-5,5,5-trifluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate (700 mg, 0.91 mmol, 1 eq) and TBAF (0.5 mL) in THF (0.5 mL) was stirred at 20° C. for 2 hours under N₂ atmosphere. After the completion of the reaction, the reaction mixture was diluted with EtOAc (50 mL) and washed with brine (50 mL). The organic phase was dried over Na₂SO₄, filtered and the filtrate was concentrated under reduced pressure. The resulting residue was purified by column chromatography to afford tert-butyl (2-(((S)-4-((tert-butoxycarbonyl)amino)-5,5,5-trifluoropentyl)oxy)pyridin-4-yl)(1-(tert-butyl)-3-((1S,3R)-3-hydroxycyclopentyl)-1H-pyrazol-5-yl)carbamate (600 mg, 94.6% yield) as a white solid. LCMS: 656.4 [M+H]⁺.

Step 8: Preparation of tert-butyl (2-(((S)-4-((tert-butoxycarbonyl)amino)-5,5,5-trifluoropentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(((S)-4-((tert-butoxycarbonyl)amino)-5,5,5-trifluoropentyl)oxy)pyridin-4-yl)(1-(tert-butyl)-3-((1S,3R)-3-hydroxycyclopentyl)-1H-pyrazol-5-yl)carbamate (500 mg, 0.76 mmol, 1 eq) and (4-nitrophenyl) carbonochloridate (461.07 mg, 2.29 mmol, 3 eq) in DCM (10 mL) was added DMAP (93.15 mg, 0.762 mmol, 1 eq) and Pyridine (603.13 mg, 7.62 mmol, 0.62 mL, 10 eq), and the mixture was stirred at 25° C. for 1 hour under N₂ atmosphere. After the completion of the reaction, the reaction mixture was diluted with EtOAc (50 mL) and washed with water (50 mL). The organic phase was dried over Na₂SO₄, filtered and the filtrate was concentrated under reduced pressure. The resulting residue was purified by column chromatography to afford tert-butyl (2-(((S)-4-((tert-butoxycarbonyl)amino)-5,5,5-trifluoropentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (600 mg, 90.1% yield) as a white solid. LCMS: 656.3 [M+H]⁺.

Step 9: Preparation of di-tert-butyl (1¹S,1³R,9S,Z)-2¹-(tert-butyl)-11-oxo-9-(trifluoromethyl)-2¹H-5,12-dioxa-3,10-diaza-4(4,2)-pyridina-2(3,5)-pyrazola-1 (1,3)-cyclopentanacyclododecaphane-3,10-dicarboxylate

To a solution of tert-butyl (2-(((S)-4-((tert-butoxycarbonyl)amino)-5,5,5-trifluoropentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (100 mg, 0.121 mmol, 1 eq) in THF (20 mL) was added NaH (9.75 mg, 0.24 mmol, 60% purity, 2 eq) at 0° C., and the mixture was then warmed to 20° C. and stirred for 1 hour under N₂ atmosphere. The reaction mixture was then quenched with saturated aqueous NH₄Cl (10 mL) at 0° C., and further diluted with water (10 mL) and extracted with EtOAc (20 mL×2). The combined organic layers were washed with brine (20 mL), dried over Na₂SO₄, filtered and the filtrate was concentrated under reduced pressure. The desired di-tert-butyl (1¹S,1³R,9S,Z)-2¹-(tert-butyl)-11-oxo-9-(trifluoromethyl)-2¹H-5,12-dioxa-3,10-diaza-4(4,2)-pyridina-2(3,5)-pyrazola-1(1,3)-cyclopentanacyclododecaphane-3,10-dicarboxylate (100 mg, 0.15 mmol, crude product) was obtained as a yellow oil, which was used into the next step without further purification. LCMS: 682.3 [M+H]⁺.

Step 10: Preparation of (1¹S,1³R,9S,Z)-9-(trifluoromethyl)-2¹H-5,12-dioxa-3,10-diaza-4(4,2)-pyridina-2(3,5)-pyrazola-1(1,3)-cyclopentanacyclododecaphan-11-one

The solution of di-tert-butyl (1¹S,1³R,9S,Z)-2¹-(tert-butyl)-11-oxo-9-(trifluoromethyl)-2¹H-5,12-dioxa-3,10-diaza-4(4,2)-pyridina-2(3,5)-pyrazola-1(1,3)-cyclopentanacyclododecaphane-3,10-dicarboxylate (50 mg, 0.073 mmol, 1 eq) in HCOOH (10 mL) was stirred at 100° C. for 1 hour under N₂ atmosphere. After the completion of the reaction, the mixture was cooled to room temperature and concentrated under reduced pressure. The resulting residue was purified by prep-HPLC (column: Waters Xbridge 150*25 mm*Sum; mobile phase: [water(NH4HCO3)-ACN]; gradient:15%-45% B over 53 min) to afford (1¹S,1³R,9S,Z)-9-(trifluoromethyl)-2¹H-5,12-dioxa-3,10-diaza-4(4,2)-pyridina-2(3,5)-pyrazola-1(1,3)-cyclopentanacyclododecaphan-11-one (1.71 mg, 5.4% yield) as a white solid. LCMS: 426.2 [M+H]⁺; ¹H NMR (400 MHz, DMSO-d₆) δ 12.09-12.06 (m, 1H), 8.85 (s, 1H) 7.88-7.72 (m, 2H), 6.43-6.36 (m, 2H), 6.08 (s, 1H), 5.17-4.93 (m, 1H), 4.19-3.98 (m, 3H), 3.26-3.23 (m, 3H), 3.17-2.33 (m, 2H), 1.80-1.75 (m, 6H) ppm.

Example 25: Synthesis of (1¹S,1³R,2⁴Z,7E,9R)-9-methyl-2¹H-5,12-dioxa-3,10-diaza-4(4,2)-pyridina-2(3,5)-pyrazola-1(1,3)-cyclopentanacyclododecaphan-7-en-11-one (compound 138)

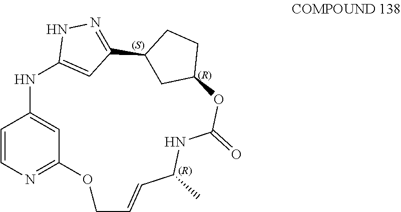

Step 1: Preparation of methyl (R,E)-4-((tert-butoxycarbonyl)amino)pent-2-enoate

A mixture of tert-butyl (R)-(1-oxopropan-2-yl)carbamate (1 g, 5.77 mmol, 1 eq), methyl 2-(triphenyl-phosphanylidene)acetate (2.12 g, 6.35 mmol, 1.1 eq) and LiOH·H₂O (726.75 mg, 17.32 mmol, 3 eq) in DCM (5 mL) was stirred at 0° C. for 3 hours under N₂ atmosphere. The mixture was concentrated in vacuo and the resulting residue was purified by column chromatography to afford 1.38 g of methyl (R,E)-4-((tert-butoxycarbonyl)amino)pent-2-enoate as a yellow oil.

Step 2: Preparation of tert-butyl (R,E)-(5-hydroxypent-3-en-2-yl)carbamate

To a solution of methyl (R,E)-4-((tert-butoxycarbonyl)amino)pent-2-enoate (1.38 g, 6.02 mmol, 1 eq) in DCM (15 mL) was added BF₃·Et₂O (865.50 mg, 6.10 mmol, 0.75 mL, 1.01 eq) dropwise −78° C. and the mixture was stirred at −78° C. for 0.5 hour under N₂ atmosphere and followed by dropwise addition of DIBAL-H (1 M, 18 mL, 2.99 eq). After the completion of the addition, the mixture was stirred at −78° C. for 2 hours under N₂ atmosphere. The mixture was subsequently quenched with 5M. AcOH (28 mL in DCM) at −78° C., warmed to room temperature and added a solution of sodium tartrate (3M., 70 mL). The mixture was extracted with DCM (150 mL×2). The combined organic layer was washed with saturated aqueous NaHCO₃(100 mL) and brine (100 mL), dried over anhydrous Na₂SO₄, filtered and the filtrate was concentrated to give 1.10 g of tert-butyl (R,E)-(5-hydroxypent-3-en-2-yl)carbamate as a yellow oil, which was used in the next step without further purification. ¹H NMR (400 MHz, CHLOROFORM-d) δ 5.73-5.66 (m, 1H), 5.64-5.59 (m, 1H), 4.39-4.32 (m, 1H), 4.25-4.16 (m, 1H), 4.07 (d, J=5.2 Hz, 2H), 1.37 (s, 9H), 1.15 (d, J=6.8 Hz, 3H) ppm.

Step 3: Preparation of tert-butyl (R,E)-(5-((4-bromopyridin-2-yl)oxy)pent-3-en-2-yl)carbamate

To a solution of tert-butyl (R,E)-(5-hydroxypent-3-en-2-yl)carbamate (640 mg, 3.18 mmol, 1.02 eq) in THF (10 mL) was added NaH (275.00 mg, 6.88 mmol, 60% purity, 2.2 eq) at 0° C., and the mixture was stirred at 25° C. for 0.5 hour, followed by the addition of 4-bromo-2-fluoro-pyridine (550 mg, 3.13 mmol, 1 eq) under N₂ atmosphere. After the completion of the addition, the mixture was warmed to 25° C. and stirred for 2 hours under N₂ atmosphere. The mixture was then quenched with saturated aqueous NH₄Cl (30 mL) and extracted with EtOAc (30 mL×2). The combined organic layers were washed with brine (50 mL), dried over anhydrous Na₂SO₄, filtered and the filtrate was concentrated. The resulting residue was purified by column chromatography to afford tert-butyl (R,E)-(5-((4-bromopyridin-2-yl)oxy)pent-3-en-2-yl)carbamate (850 mg, 76.1% yield) as a colorless oil. LCMS: 359.1 [M+H]⁺, 303.1 [M−55+H]⁺; ¹H NMR (400 MHz, CHLOROFORM-d) δ 7.99 (d, J=5.2 Hz, 1H), 7.05 (d, J=5.2 Hz, 1H), 6.98 (s, 1H), 5.84 (s, 2H), 4.83-4.82 (m, 2H), 4.49-4.47 (m, 1H), 4.34-4.30 (m, 1H), 1.46 (s, 9H), 1.26 (d, J=6.8 Hz, 3H) ppm.

Step 4: Preparation of tert-butyl ((R,E)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)pent-3-en-2-yl)carbamate

A mixture of tert-butyl (R,E)-(5-((4-bromopyridin-2-yl)oxy)pent-3-en-2-yl)carbamate (500 mg, 1.40 mmol, 1 eq), 1-(tert-butyl)-3-((1S,3R)-3-((tert-butyldimethylsilyl)oxy)cyclopentyl)-1H-pyrazol-5-amine (475.00 mg, 1.41 mmol, 1.01 eq), Cs₂CO₃ (1.37 g, 4.20 mmol, 3 eq), Pd₂(dba)₃ (128.16 mg, 0.14 mmol, 0.1 eq) and Xantphos (161.97 mg, 0.28 mmol, 0.2 eq) in dioxane (10 mL) was degassed and back-filled with N₂ for three times, and stirred at 90° C. for 3 hours under N₂ atmosphere. After the completion of the reaction, the mixture was cooled to room temperature. The mixture was filtered through a pad of the Celite, rinsed with ethyl acetate (20 mL) and the combined filtrate was concentrated in vacuo. The resulting residue was purified by column chromatography to afford tert-butyl ((R,E)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)pent-3-en-2-yl)carbamate (395 mg, 41.9% yield) as a colorless oil. LCMS: 614.5 [M+H]⁺.

Step 5: Preparation of of tert-butyl (2-(((R,E)-4-((tert-butoxycarbonyl)amino)pent-2-en-1-yl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((R,E)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)pent-3-en-2-yl)carbamate (395 mg, 0.59 mmol, 1 eq) in DCM (10 mL) were added DMAP (60 mg, 0.49 mmol, 0.84 eq) and Boc₂O (257 mg, 1.18 mmol, 2.01 eq), and the reaction mixture was stirred at 25° C. for 12 hours under N₂ atmosphere. After the completion of the reaction, the mixture was quenched with 0.5N HCl (30 mL) and extracted with EtOAc (30 mL×2). The combined organic layers were washed with H₂O (30 mL) and brine (30 mL), dried over anhydrous Na₂SO₄, filtered and the filtrate was concentrated to give 510 mg of tert-butyl (2-(((R,E)-4-((tert-butoxycarbonyl)amino)pent-2-en-1-yl)oxy)pyridin-4-yl)(1-(tert-butyl)-3-((1S,3R)-3-((tert-butyldimethylsilyl)oxy)cyclopentyl)-1H-pyrazol-5-yl)carbamate as a colorless oil, which was used in the next step without further purification. LCMS: 714.4 [M+H]⁺.

Step 6: Preparation tert-butyl (2-(((R,E)-4-((tert-butoxycarbonyl)amino)pent-2-en-1-yl)oxy)pyridin-4-yl)(1-(tert-butyl)-3-((1S,3R)-3-hydroxycyclopentyl)-1H-pyrazol-5-yl)carbamate

The solution of tert-butyl (2-(((R,E)-4-((tert-butoxycarbonyl)amino)pent-2-en-1-yl)oxy)pyridin-4-yl)(1-(tert-butyl)-3-((1S,3R)-3-((tert-butyldimethylsilyl)oxy)cyclopentyl)-1H-pyrazol-5-yl)carbamate (510 mg, 0.71 mmol, 1 eq) and TBAF (1 M, 5 mL, 7 eq) in THF (5 mL) was stirred at 25° C. for 3 hours under N₂ atmosphere. After the completion of the reaction, the mixture was quenched with H₂O (20 mL) and extracted with EtOAc (30 mL×2). The combined organic layers were washed with brine (50 mL), dried over anhydrous Na₂SO₄, filtered and the filtrate was concentrated to give tert-butyl (2-(((R,E)-4-((tert-butoxycarbonyl)amino)pent-2-en-1-yl)oxy)pyridin-4-yl)(1-(tert-butyl)-3-((1S,3R)-3-hydroxycyclopentyl)-1H-pyrazol-5-yl)carbamate (500 mg, 0.83 mmol) as a yellow oil, which was used into the next step without further purification. LCMS: 600.3 [M+H]⁺.

Step 7: Preparation of tert-butyl (2-(((R,E)-4-((tert-butoxycarbonyl)amino)pent-2-en-1-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(((R,E)-4-((tert-butoxycarbonyl)amino)pent-2-en-1-yl)oxy)pyridin-4-yl)(1-(tert-butyl)-3-((1S,3R)-3-hydroxycyclopentyl)-1H-pyrazol-5-yl)carbamate (500 mg, 0.83 mmol, 1 eq), 4-nitrophenyl carbonochloridate (500.00 mg, 2.48 mmol, 2.98 eq) and DMAP (55.00 mg, 0.45 mmol, 0.54 eq) in THF (10 mL) was added pyridine (490.00 mg, 6.19 mmol, 0.5 mL, 7.43 eq) dropwise, and the reaction mixture was stirred at 25° C. for 0.5 hour under N₂ atmosphere. After the completion of the reaction, the mixture was filtered, rinsed with THF (10 mL) and the combined filtrate was concentrated in vacuo. The resulting residue was purified by column chromatography to afford tert-butyl (2-(((R,E)-4-((tert-butoxycarbonyl)amino)pent-2-en-1-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (470 mg, 73.7% yield) as a colorless oil. LCMS: 765.3 [M+H]⁺.

Step 8: Preparation of (1R,3S)-3-(5-((2-(((R,E)-4-aminopent-2-en-1-yl)oxy)pyridin-4-yl)amino)-1-(tert-butyl)-1H-pyrazol-3-yl)cyclopentyl (4-nitrophenyl) carbonate

The solution of tert-butyl (2-(((R,E)-4-((tert-butoxycarbonyl)amino)pent-2-en-1-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (270 mg, 0.35 mmol, 1 eq) and TFA (1 mL) in DCM (5 mL) was stirred at 25° C. for 5 hours under N₂ atmosphere. The mixture was concentrated in vacuo to give 240 mg of (1R,3S)-3-(5-((2-(((R,E)-4-aminopent-2-en-1-yl)oxy)pyridin-4-yl)amino)-1-(tert-butyl)-1H-pyrazol-3-yl)cyclopentyl (4-nitrophenyl) carbonate as a yellow oil, which was used in the next step without further purification. LCMS: 565.3 [M+H]⁺.

Step 9: Preparation of (1¹S,1³R,2⁴Z,7E,9R)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-7-en-11-one

To a solution of (1R,3S)-3-(5-((2-(((R,E)-4-aminopent-2-en-1-yl)oxy)pyridin-4-yl)amino)-1-(tert-butyl)-1H-pyrazol-3-yl)cyclopentyl (4-nitrophenyl) carbonate (240 mg, 0.35 mmol, 1 eq) in THF (150 mL) was added DIEA (1.11 g, 8.61 mmol, 1.5 mL, 24.35 eq), and the reaction mixture was stirred at 80° C. for 12 hours under N₂ atmosphere. After the completion of the reaction, the mixture was cooled to room temperature. The mixture was concentrated in vacuo to afford (1¹S,1³R,2⁴Z,7E,9R)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-7-en-11-one (150 mg, 0.35 mmol) as a yellow oil, which was used in the next step without further purification. LCMS: 426.3 [M+H]⁺.

Step 10: Preparation of (1¹S,1³R,2⁴Z,7E,9R)-9-methyl-2¹H-5,12-dioxa-3,10-diaza-4(4,2)-pyridina-2(3,5)-pyrazola-1(1,3)-cyclopentanacyclododecaphan-7-en-11-one

A mixture of (1¹S,1³R,2⁴Z,7E,9R)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-7-en-11-one (150 mg, 0.35 mmol, 1 eq) and formic acid (6 mL) was stirred at 100° C. for 2 hours under N₂ atmosphere. After the completion of the reaction, the mixture was cooled to room temperature, and then concentrated under reduced pressure. The resulting residue was purified by prep-HPLC {column: Phenomenex luna C18 150*25 mm*10 um; mobile phase: [water(FA)-ACN]; gradient:5%-35% B over 10 min}, followed by SFC {column: DAICEL CHIRALCEL OJ-H(250 mm*30 mm, 5 um); mobile phase: [CO₂-EtOH(0.1% NH₃·H₂O)]; B %:20%, isocratic elution mode} to afford title compound (1¹S,1³R,2⁴Z,7E,9R)-9-methyl-2¹H-5,12-dioxa-3,10-diaza-4(4,2)-pyridina-2(3,5)-pyrazola-1(1,3)-cyclopentanacyclododecaphan-7-en-11-one (22 mg, 15.0% yield) as a white solid. LCMS: 370.1 [M+H]⁺; ¹H NMR (400 MHz, DMSO-d₆) δ 12.26-12.04 (m, 1H), 9.08-8.81 (m, 1H), 8.56-8.51 (m, 1H), 7.74 (d, J=5.6 Hz, 1H), 6.90-6.77 (m, 1H), 6.43-6.42 (m, 1H), 6.31 (s, 1H), 5.89-5.87 (m, 1H), 5.76-5.66 (m, 1H), 5.65-5.57 (m, 1H), 5.26-5.16 (m, 1H), 4.68-4.58 (m, 2H), 4.13-4.06 (m, 1H), 2.07-2.02 (m, 1H), 1.96-1.73 (m, 5H), 1.30-1.24 (m, 1H), 1.12 (br s, 3H) ppm.

Example 26: synthesis of (1¹S,1³R,7¹S,7³S,Z)-2¹H-10-oxa-3,8-diaza-4(4,2)-pyridina-2(3,5)-pyrazola-1(1,3)-cyclopentana-7(1,3)-cyclobutanacyclodecaphan-9-one(compound 139) and (1¹S,1³R,7¹R,7³R,Z)-2¹H-10-oxa-3,8-diaza-4(4,2)-pyridina-2(3,5)-pyrazola-1(1,3)-cyclopentana-7(1,3)-cyclobutanacyclodecaphan-9-one (compound 140)

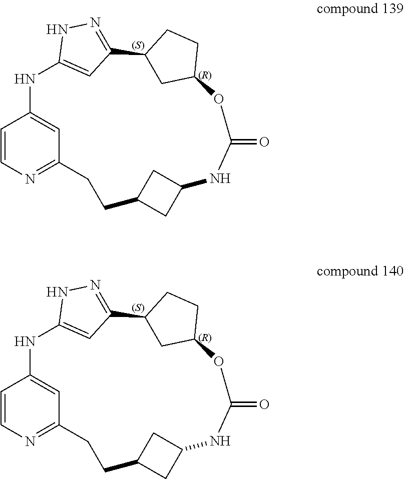

Step 1: Preparation of tert-butyl ((1r,3r)-3-formylcyclobutyl)carbamate

To a solution of tert-butyl ((1r,3r)-3-(hydroxymethyl)cyclobutyl)carbamate (2.00 g, 9.94 mmol, 1 eq) in DCM (40 mL) was added dropwise DIEA (7.71 g, 59.62 mmol, 10.39 mL, 6 eq), sulfur trioxide pyridine complex (4.74 g, 29.81 mmol, 3 eq) and DMSO (9.32 g, 119.25 mmol, 12 eq) at −10° C. After the completion of the addition, the mixture was warmed to 0° C. and stirred for 2 hours under N₂ atmosphere. The mixture was diluted with 10% citric acid (60 mL) and extracted with EtOAc (100 mL×2). The combined organic layers were washed with brine (50 mL×2), dried over Na₂SO₄, filtered, and the filtrate was concentrated under reduced pressure. The resulting residue was purified by column chromatography to give tert-butyl ((1r,3r)-3-formylcyclobutyl)carbamate (1.4 g, 70.7% yield) as a white solid.

Step 2: Preparation of tert-butyl ((1r,3r)-3-ethynylcyclobutyl)carbamate

A mixture of tert-butyl ((1r,3r)-3-formylcyclobutyl)carbamate (600 mg, 3.01 mmol, 1 eq), 1-diazo-1-dimethoxyphosphoryl-propan-2-one (578.51 mg, 3.01 mmol, 1 eq) and K₂CO₃ (832.37 mg, 6.02 mmol, 2 eq) in MeOH (6 mL) was stirred at 25° C. for 3 hours under N₂ atmosphere. After the completion of the reaction, the mixture was filtered through a pad of the Celite, rinsed with MeOH (10 mL) and the filtrate was concentrated in vacuo. The resulting residue was purified by prep-TLC (PE/EtOAc=2/1) to give tert-butyl ((1r,3r)-3-ethynylcyclobutyl)carbamate (525 mg, 89.2% yield) as a yellow oil. ¹H NMR (400 MHz, CHLOROFORM-d) δ=4.80-4.65 (m, 1H), 4.43-4.28 (m, 1H), 4.15-3.95 (m, 1H), 3.10-2.85 (m, 1H), 2.75-2.60 (m, 1H), 2.55-2.40 (m, 2H), 2.25-2.10 (m, 3H), 2.10-1.90 (m, 1H), 1.49-1.41 (m, 9H) ppm.

Step 3: Preparation of tert-butyl ((1r,3r)-3-((4-bromopyridin-2-yl)ethynyl)cyclobutyl)carbamate

A solution of of tert-butyl ((1r,3r)-3-ethynylcyclobutyl)carbamate (475 mg, 2.43 mmol, 1 eq) and 2,4-dibromopyridine (576.28 mg, 2.43 mmol, 1 eq) in DMF (15 mL) was degassed and backfilled with N₂ for 3 times. To the solution were added CuI (138.99 mg, 0.73 mmol, 0.3 eq), Pd(PPh₃)₄ (421.67 mg, 0.36 mmol, 0.15 eq) and TEA (738.48 mg, 7.30 mmol, 1.02 mL, 3 eq) under N₂ atmosphere and the mixture was stirred at 40° C. for 3 hours. After the completion of the reaction, the mixture was cooled to room temperature, and filtered through a pad of the Celite, rinsed with ethyl acetate (30 mL). The combined filtrate was concentrated in vacuo, and the resulting residue was purified by prep-TLC (PE/EtOAc=1/1) to afford tert-butyl ((1r,3r)-3-((4-bromopyridin-2-yl)ethynyl)cyclobutyl)carbamate (714 mg, 2.03 mmol, 83.5% yield) as a yellow oil. LCMS: 295.0/297.0 [M−56]⁺.

Step 4: Preparation of tert-butyl ((1S,3r)-3-((4-((1-(tert-butyl)-3-((1S,3R)-3-((tert-butyldimethylsilyl)oxy)cyclopentyl)-1H-pyrazol-5-yl)amino)pyridin-2-yl)ethynyl)cyclobutyl)carbamate

A mixture of tert-butyl ((1r,3r)-3-((4-bromopyridin-2-yl)ethynyl)cyclobutyl)carbamate (614 mg, 1.75 mmol, 1 eq), 1-(tert-butyl)-3-((1S,3R)-3-((tert-butyldimethylsilyl)oxy)cyclopentyl)-1H-pyrazol-5-amine (649.13 mg, 1.92 mmol, 1.1 eq), Cs₂CO₃ (1.71 g, 5.24 mmol, 3 eq), Xantphos (121.38 mg, 0.209 mmol, 0.12 eq), and Pd₂(dba)₃(100.52 mg, 0.174 mmol, 0.1 eq) in dioxane (15 mL) was degassed and backfilled with N₂ for 3 times, and stirred at 90° C. for 3 hours under N₂ atmosphere. After the completion of the reaction, the mixture was cooled to room temperature. The mixture was filtered through a pad of the Celite, rinsed with dioxane (10 mL) and the filtrate was concentrated in vacuo. The resulting residue was purified by prep-TLC to give tert-butyl ((1S,3r)-3-((4-((1-(tert-butyl)-3-((1S,3R)-3-((tert-butyldimethylsilyl)oxy)cyclopentyl)-1H-pyrazol-5-yl)amino)pyridin-2-yl)ethynyl)cyclobutyl)carbamate (1 g, 94.1% yield) as a yellow oil. LCMS: 608.5 [M+H]⁺.

Step 5: Preparation of tert-butyl ((1R,3r)-3-(2-(4-((1-(tert-butyl)-3-((1S,3R)-3-((tert-butyldimethylsilyl)oxy)cyclopentyl)-1H-pyrazol-5-yl)amino)pyridin-2-yl)ethyl)cyclobutyl)carbamate

The mixture of tert-butyl ((1S,3r)-3-((4-((1-(tert-butyl)-3-((1S,3R)-3-((tert-butyldimethylsilyl)oxy)cyclopentyl)-1H-pyrazol-5-yl)amino)pyridin-2-yl)ethynyl)cyclobutyl)carbamate (1 g, 1.65 mmol, 1 eq) and Pt/C (641.81 mg, 0.164 mmol, 5% purity, 0.1 eq) in THF (15 mL) was degassed and backfilled with H₂ for 3 times, and the mixture was stirred at 40° C. under H₂ atmosphere for 3 hours. After the completion of the reaction, the mixture was cooled to room temperature, and filtered through a pad of the Celite, rinsed with THF (10 mL). The filtrate was concentrated under reduced pressure to give tert-butyl ((1R,3r)-3-(2-(4-((1-(tert-butyl)-3-((1S,3R)-3-((tert-butyldimethylsilyl)oxy)cyclopentyl)-1H-pyrazol-5-yl)amino)pyridin-2-yl)ethyl)cyclobutyl)carbamate (1 g, 1.63 mmol) as a yellow oil, which was used in the next step without further purification. LCMS: 612.6 [M+H]⁺.

Step 6: Preparation of tert-butyl (2-(2-((1r,3R)-3-((tert-butoxycarbonyl)amino)cyclobutyl)ethyl)pyridin-4-yl)(1-(tert-butyl)-3-((1S,3R)-3-((tert-butyldimethylsilyl)oxy)cyclopentyl)-1H-pyrazol-5-yl)carbamate

To a mixture of tert-butyl ((1R,3r)-3-(2-(4-((1-(tert-butyl)-3-((1S,3R)-3-((tert-butyldimethylsilyl)oxy)cyclopentyl)-1H-pyrazol-5-yl)amino)pyridin-2-yl)ethyl)cyclobutyl)carbamate (1 g, 1.63 mmol, 1 eq) and Boc₂O (713.31 mg, 3.27 mmol, 2 eq) in DCM (15 mL) was added DMAP (99.82 mg, 0.817 mmol, 0.5 eq), and the mixture was stirred at 25° C. for 12 hours under N₂ atmosphere. After the completion of the reaction, the mixture was concentrated. The resulting residue was purified by column chromatography to give tert-butyl (2-(2-((1r,3R)-3-((tert-butoxycarbonyl)amino)cyclobutyl)ethyl)pyridin-4-yl)(1-(tert-butyl)-3-((1S,3R)-3-((tert-butyldimethylsilyl)oxy)cyclopentyl)-1H-pyrazol-5-yl)carbamate (500 mg, 42.9% yield) as a yellow oil. LCMS: 712.6 [M+H]⁺.

Step 7: Preparation of tert-butyl (2-(2-((1r,3R)-3-((tert-butoxycarbonyl)amino)cyclobutyl)ethyl)pyridin-4-yl)(1-(tert-butyl)-3-((1S,3R)-3-hydroxycyclopentyl)-1H-pyrazol-5-yl)carbamate

The mixture of tert-butyl (2-(2-((1r,3R)-3-((tert-butoxycarbonyl)amino)cyclobutyl)ethyl)pyridin-4-yl)(1-(tert-butyl)-3-((1S,3R)-3-((tert-butyldimethylsilyl)oxy)cyclopentyl)-1H-pyrazol-5-yl)carbamate (450 mg, 0.631 mmol, 1 eq) and TBAF (991.44 mg, 0.631 mmol, 6 eq) in THF (10 mL) was stirred at 25° C. for 1 hour under N₂ atmosphere. After the completion of the reaction, the reaction mixture was diluted with saturated aqueous NH₄Cl (50 mL) and extracted with EtOAc (20 mL×3). The combined organic layers were washed with brine (50 mL), dried over Na₂SO₄, filtered, and the filtrate was concentrated under reduced pressure. The title compound tert-butyl (2-(2-((1r,3R)-3-((tert-butoxycarbonyl)amino)cyclobutyl)ethyl)pyridin-4-yl)(1-(tert-butyl)-3-((1S,3R)-3-hydroxycyclopentyl)-1H-pyrazol-5-yl)carbamate (360 mg, 0.602 mmol) was obtained as a yellow oil, which was used in the next step without further purification. LCMS: 598.4 [M+H]⁺.

Step 8: Preparation of tert-butyl (2-(2-((1r,3R)-3-((tert-butoxycarbonyl)amino)cyclobutyl)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(2-((1r,3R)-3-((tert-butoxycarbonyl)amino)cyclobutyl)ethyl)pyridin-4-yl)(1-(tert-butyl)-3-((1S,3R)-3-hydroxycyclopentyl)-1H-pyrazol-5-yl)carbamate (359 mg, 0.6 mmol, 1 eq) in DCM (5 mL) was added (4-nitrophenyl) carbonochloridate (363.15 mg, 1.80 mmol, 3 eq), DMAP (36.68 mg, 0.3 mmol, 0.5 eq) and Pyridine (285.02 mg, 3.60 mmol, 6 eq), and the mixture was stirred at 25° C. for 4 hours under N₂ atmosphere. After the completion of the reaction, the reaction mixture was quenched with saturated aqueous NH₄Cl (50 mL) and extracted with EtOAc (20 mL×3). The combined organic layers were washed with brine (50 mL), dried over Na₂SO₄, filtered and the filtrate was concentrated under reduced pressure. The resulting residue was purified by column chromatography to give tert-butyl (2-(2-((1r,3R)-3-((tert-butoxycarbonyl)amino)cyclobutyl)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate (450 mg, 98.2% yield) as a yellow oil. LCMS: 763.4 [M+H]⁺.

Step 9: Preparation of (1R,3S)-3-(5-((2-(2-((1r,3R)-3-aminocyclobutyl)ethyl)pyridin-4-yl)amino)-1-(tert-butyl)-1H-pyrazol-3-yl)cyclopentyl (4-nitrophenyl) carbonate

A mixture of tert-butyl (2-(2-((1r,3R)-3-((tert-butoxycarbonyl)amino)cyclobutyl)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate (400 mg, 0.524 mmol, 1 eq) and TFA (179.35 mg, 1.57 mmol, 3 eq) in DCM (2 mL) was stirred at 25° C. for 3 hours under N₂ atmosphere. After the completion of the reaction, the mixture was concentrated to give 295 mg of (1R,3S)-3-(5-((2-(2-((1r,3R)-3-aminocyclobutyl)ethyl)pyridin-4-yl)amino)-1-(tert-butyl)-1H-pyrazol-3-yl)cyclopentyl (4-nitrophenyl) carbonate as a yellow oil, which was used in next step without further purification. LCMS: 563.5 [M+H]⁺.

Step 10: Preparation of (1¹S,1³R,7¹R,7³R,Z)-2¹-(tert-butyl)-2¹H-10-oxa-3,8-diaza-4(4,2)-pyridina-2(3,5)-pyrazola-1(1,3)-cyclopentana-7(1,3)-cyclobutanacyclodecaphan-9-one

A mixture of (1R,3S)-3-(5-((2-(2-((1r,3R)-3-aminocyclobutyl)ethyl)pyridin-4-yl)amino)-1-(tert-butyl)-1H-pyrazol-3-yl)cyclopentyl (4-nitrophenyl) carbonate (281 mg, 0.499,mmol, 1 eq) and DIEA (322.73 mg, 2.50 mmol, 5 eq) in THF (280 mL) was stirred at 25° C. for 3 hours under N₂ atmosphere. After the completion of the reaction, the mixture was concentrated to give (1¹S,1³R,7¹R,7³R,Z)-2¹-(tert-butyl)-2¹H-10-oxa-3,8-diaza-4(4,2)-pyridina-2(3,5)-pyrazola-1(1,3)-cyclopentana-7(1,3)-cyclobutanacyclodecaphan-9-one (211 mg, 0.498 mmol) as an yellow oil, which was used in the next step without further purification. LCMS: 424.3 [M+H]⁺.

Step 11: Preparation of (1¹S,1³R,7¹S,7³S,Z)-2¹H-10-oxa-3,8-diaza-4(4,2)-pyridina-2(3,5)-pyrazola-1(1,3)-cyclopentana-7(1,3)-cyclobutanacyclodecaphan-9-one (compound 139) and (1¹S,1³R,7¹R,7³R,Z)-2¹H-10-oxa-3,8-diaza-4(4,2)-pyridina-2(3,5)-pyrazola-1(1,3)-cyclopentana-7(1,3)-cyclobutanacyclodecaphan-9-one (compound 140)

A mixture of (1¹S,1³R,7¹R,7³R,Z)-2¹-(tert-butyl)-2¹H-10-oxa-3,8-diaza-4(4,2)-pyridina-2(3,5)-pyrazola-1(1,3)-cyclopentana-7(1,3)-cyclobutanacyclodecaphan-9-one (211 mg, 0.498 mmol, 1 eq) in HCOOH (5 mL) was stirred at 100° C. for 3 hours under N₂ atmosphere. After the completion of the reaction, the mixture was cooled to room temperature and concentrated under reduced pressure. The resulting residue was purified by prep-HPLC {column: Waters Xbridge 150*25 mm*Sum; mobile phase: [column: Waters Xbridge 150*25 mm*Sum; mobile phase: [water(NH₄HCO3)-ACN]; gradient:15%-45% B over 53 min} and further by SFC{column: DAICEL CHIRALPAK AD (250 mm*30 mm, 10 um); mobile phase: [CO₂-EtOH(0.1% NH₃·H₂O)]; B %:40%, isocratic elution mode} to give (1¹S,1³R,7¹S,7³S,Z)-2¹H-10-oxa-3,8-diaza-4(4,2)-pyridina-2(3,5)-pyrazola-1(1,3)-cyclopentana-7(1,3)-cyclobutanacyclodecaphan-9-one (compound 139) (20.07 mg, 0.542 mmol, 10.9% yield, 99.4% purity) as a white solid and (1¹S,1³R,7¹R,7³R,Z)-2¹H-10-oxa-3,8-diaza-4(4,2)-pyridina-2(3,5)-pyrazola-1(1,3)-cyclopentana-7(1,3)-cyclobutanacyclodecaphan-9-one (compound 140) (31.58 mg, 0.845 mmol, 16.9% yield, 98.4% purity) as a white solid.

(1¹S,13R,7¹S,7³S,Z)-2¹H-10-oxa-3,8-diaza-4(4,2)-pyridina-2(3,5)-pyrazola-1(1,3)-cyclopentana-7(1,3)-cyclobutanacyclodecaphan-9-one (compound 139) LCMS: 368.2 [M+H]⁺; ¹H NMR (400 MHz, DMSO-d₆) 5=12.80-11.50 (m, 1H), 9.20-8.48 (m, 1H), 8.15-7.88 (m, 1H), 7.65-6.99 (m, 1H), 6.95-6.48 (m, 2H), 6.08-5.83 (m, 1H), 5.18-4.89 (m, 1H), 3.19 (br d, J=4.0 Hz, 1H), 2.52-2.51 (m, 1H), 2.46-2.40 (m, 2H), 2.37-1.28 (m, 13H) ppm.

(1¹S,1³R,7¹R,7³R,Z)-2¹H-10-oxa-3,8-diaza-4(4,2)-pyridina-2(3,5)-pyrazola-1(1,3)-cyclopentana-7(1,3)-cyclobutanacyclodecaphan-9-one (compound 140) LCMS: 368.2 [M+H]⁺; ¹H NMR (400 MHz, DMSO-d₆) 5=12.77-11.90 (m, 1H), 9.31 (s, 1H), 8.13-7.83 (m, 1H), 7.62-6.99 (m, 1H), 6.93-6.49 (m, 2H), 6.07-5.84 (m, 1H), 5.12-4.89 (m, 1H), 4.13-3.90 (m, 1H), 3.30-3.11 (m, 2H), 2.36-1.46 (m, 13H) ppm.

Example 27: synthesis of (1¹S,1³R,7¹R,7³S,6S,Z)-6-methyl-2¹H-5,10-dioxa-3,8-diaza-4(4,2)-pyridina-2(3,5)-pyrazola-1(1,3)-cyclopentana-7(1,3)-cyclobutanacyclodecaphan-9-one (compound 146)

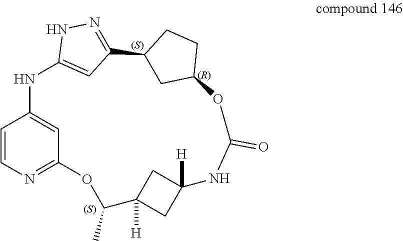

Step 1: Preparation of (S)-((but-3-yn-2-yloxy)methyl)benzene

To a solution of (S)-but-3-yn-2-ol (25 g, 356.69 mmol, 1.53 eq) in THF (400 mL) was added NaH (9.35 g, 233.87 mmol, 60% purity, 1 eq) at 0° C. under N₂ atmosphere. After the mixture was stirred at 0° C. for 0.5 hour under N₂ atmosphere, NaI (3.51 g, 23.39 mmol, 0.1 eq) and BnBr (40 g, 233.87 mmol, 27.78 mL, 1 eq) were subsequently added, and the mixture was kept stirring at 25° C. for 16 hours under N₂ atmosphere. The mixture was then quenched with saturated aqueous NH₄Cl (200 mL) and extracted with EtOAc (200 mL×2). The combined organic layers were washed with brine (300 mL), dried over anhydrous Na₂SO₄, filtered and the filtrate was concentrated in vacuo. The resulting residue was purified by column chromatography to afford (S)-((but-3-yn-2-yloxy)methyl)benzene (40 g, 249.67 mmol) as a yellow oil.

Step 2: Preparation of (S)-((but-3-en-2-yloxy)methyl)benzene

A suspension of (S)-((but-3-yn-2-yloxy)methyl)benzene (100 g, 624.14 mmol) in MeOH (4 L) was stirred at 20° C. until a clear solution was formed. The clear solution was pumped at a flow rate of 0.9 mL/min into the reactor in which the H₂ back pressure regulator was adjusted to 0.5 MPa, the flow rate of H₂ was at 30 mL/min and the fixed bed (5% Ni/Al₂O₃, 15 g) was heated to 55° C. After the completion of the reaction, the tubing was washed with MeOH (100 mL), and all the reaction solution was collected for analysis. The solution was concentrated in vacuo. The resulting residue was purified by column chromatography to afford (S)-((but-3-en-2-yloxy)methyl)benzene (42 g, 258.89 mmol, 41.5% yield) as a colorless oil. ¹H NMR (400 MHz, CHLOROFORM-d) δ 7.26-7.17 (m, 5H), 5.73-5.66 (m, 1H), 5.15-5.07 (m, 2H), 4.50-4.29 (m, 2H), 3.85-3.82 (m, 3H), 1.22-1.20 (d, J=5.2 Hz, 3H) ppm.

Step 3: Preparation of 3-((S)-1-(benzyloxy)ethyl)-2,2-dichlorocyclobutan-1-one

A mixture of (S)-((but-3-en-2-yloxy)methyl)benzene (42 g, 258.89 mmol, 1 eq) and Zn—Cu (83.5 g, 0.65 mmol, 2.5 eq) in DME (500 mL) was pumped into the reactor at a flow rate of 6.2 mL/min. Meanwhile, another solution of 2,2,2-trichloroacletyl chloride(285.3 g, 1.57 mmol, 6 eq) in DME (500 mL) was pumped into the reactor at a flow rate of 6.2 mL/min. The mixture was run in a flow reaction at 40° C. under N₂ atmosphere. After the completion of the reaction, the mixture was filtered through a Büchner funnel, and washed with EtOAc (500 mL×3). The filtrate was washed with brine (500 mL), saturated aqueous NaHCO₃(100 mL) and brine (100 mL). The organic layer was dried over anhydrous Na₂SO₄, filtered, and concentrated in vacuo. The resulting residue was purified by column chromatography to afford 3-((S)-1-(benzyloxy)ethyl)-2,2-dichlorocyclobutan-1-one (16 g, 22.7% yield) as a colorless oil. ¹H NMR (400 MHz, CHLOROFORM-d) δ 7.45-7.32 (m, 5H), 4.71-4.61 (m, 2H), 3.96-3.94 (m, 1H), 3.26-3.21 (m, 1H), 3.09-3.06 (m, 2H), 1.32-1.31 (d, J=5.2 Hz, 3H) ppm.

Step 4: Preparation of (S)-3-(1-(benzyloxy)ethyl)cyclobutan-1-one

To a solution of 3-((S)-1-(benzyloxy)ethyl)-2,2-dichlorocyclobutan-1-one (7.4 g, 27.09 mmol, 1 eq) in MeOH (100 mL) were added NH₄Cl (8.69 g, 162.55 mmol, 6 eq) and Zn powder (9.08 g, 162.55 mmol, 6 eq), and the mixture was stirred at 20° C. for 12 hours under N₂ atmosphere. After the completion of the reaction, the mixture was filtered through a pad of the Celite, rinsed with methanol and the filtrate was concentrated under reduced pressure. The resulting residue was diluted with EtOAc (100 mL), washed with saturated aqueous NaHCO₃(100 mL) and brine (100 mL). The organic layer was dried over anhydrous Na₂SO₄, filtered and the filtrate was concentrated in vacuo. The resulting crude was purified by column chromatography to afford (S)-3-(1-(benzyloxy)ethyl)cyclobutan-1-one (4.07 g, 33.9% yield) as a colorless oil. ¹H NMR (400 MHz, CHLOROFORM-d) δ 7.39-7.31 (m, 5H), 4.70 (d, J=11.6 Hz, 1H), 4.46 (d, J=11.6 Hz, 1H), 3.66-3.60 (m, 1H), 3.08-3.02 (m, 3H), 2.93-2.87 (m, 1H), 2.54-2.45 (m, 1H), 1.27 (d, J=6.0 Hz, 3H) ppm.

Step 5: Preparation of (1R,3s)-3-((S)-1-(benzyloxy)ethyl)cyclobutan-1-ol

To a solution of (S)-3-(1-(benzyloxy)ethyl)cyclobutan-1-one (3.57 g, 17.48 mmol, 1 eq) in THF (50 mL) at −78° C. was added L-selectride (1 M, 20 mL, 1.14 eq), and the mixture was stirred at −78° C. for 2 hours under N₂ atmosphere. After the completion of the reaction, the mixture was quenched with saturated aqueous NH₄Cl (100 mL) and extracted with EtOAc (60 mL×2). The combined organic layers were washed with brine (100 mL), dried over anhydrous Na₂SO₄, filtered and the filtrate was concentrated in vacuo. The desired (1R,3s)-3-((S)-1-(benzyloxy)ethyl)cyclobutan-1-ol (3.62 g, crude) was obtained as a colorless oil, which was used in the next step without further purification. ¹H NMR (400 MHz, CHLOROFORM-d) δ 7.37-7.35 (m, 4H), 7.32-7.30 (m, 1H), 4.64 (d, J=12.0 Hz, 1H), 4.48 (d, J=11.6 Hz, 1H), 4.17-4.11 (m, 1H), 3.78-3.73 (m, 1H), 3.46-3.43 (m, 1H), 2.55-2.49 (m, 1H), 2.40-2.36 (m, 1H), 1.92-1.87 (m, 1H), 1.77-1.67 (m, 2H), 1.13-1.12 (d, J=6.0 Hz, 3H) ppm.

Step 6: Preparation of (1R,3s)-3-((S)-1-(benzyloxy)ethyl)cyclobutyl 4-methylbenzenesulfonate

To a solution of (1R,3s)-3-((S)-1-(benzyloxy)ethyl)cyclobutan-1-ol (3.62 g, 17.55 mmol, 1 eq) and TsCl (5.02 g, 26.32 mmol, 1.5 eq) in DCM (100 mL) were added DMAP (330 mg, 2.70 mmol, 0.15 eq) and TEA (5.45 g, 53.88 mmol, 7.5 mL, 3.07 eq) at 0° C., and the mixture was warmed to 25° C. and stirred at that temperature for 2 hours. After the completion of the reaction, the mixture was quenched with saturated aqueous NaHCO₃(50 mL) and extracted with DCM (50 mL×2). The combined organic layers were washed with brine (100 mL), dried over anhydrous Na₂SO₄, filtered and the filtrate was concentrated in vacuo. The resulting residue was purified by column chromatography to afford (1R,3s)-3-((S)-1-(benzyloxy)ethyl)cyclobutyl 4-methylbenzenesulfonate (5.1 g, 44.3% yield) as a colorless oil. LCMS: 378.2 [M+H₂O]+; ¹H NMR (400 MHz, CHLOROFORM-d) δ 7.80 (d, J=8.4 Hz, 2H), 7.36-7.30 (m, 7H), 4.71-4.64 (m, 1H), 4.58 (d, J=11.6 Hz, 1H), 4.41 (d, J=12.0 Hz, 1H), 3.42-3.35 (m, 1H), 2.47 (s, 3H), 2.41-2.33 (m, 1H), 2.29-2.22 (m, 1H), 2.07-1.84 (m, 3H), 1.07 (d, J=6.4 Hz, 3H) ppm.

Step 7: Preparation of (((S)-1-((1r,3S)-3-azidocyclobutyl)ethoxy)methyl)benzene

To a solution of (1R,3s)-3-((S)-1-(benzyloxy)ethyl)cyclobutyl 4-methylbenzenesulfonate (5.1 g, 14.15 mmol, 1 eq) in DMF (60 mL) was added NaN₃ (1.96 g, 30.15 mmol, 2.13 eq), and the mixture was stirred at 100° C. for 16 hours under N₂ atmosphere. After the completion of the reaction, the mixture was cooled to room temperature, quenched with saturated aqueous NaHCO₃(100 mL) and extracted with EtOAc (50 mL×2). The combined organic layers were washed with H₂O (60 mL×2) and brine (100 mL), dried over anhydrous Na₂SO₄, filtered, and the filtrate was concentrated in vacuo. The resulting residue was purified by column chromatography to afford (((S)-1-((1r,3S)-3-azidocyclobutyl)ethoxy)methyl)benzene (2.84 g, 86.8% yield) as a colorless oil. ¹H NMR (400 MHz, CHLOROFORM-d) δ 7.40-7.33 (m, 4H), 7.32-7.29 (m, 1H), 4.67 (d, J=12.0 Hz, 1H), 4.44 (d, J=12.0 Hz, 1H), 4.01-3.95 (m, 1H), 3.55-3.49 (m, 1H), 2.44-2.33 (m, 2H), 2.22-2.16 (m, 3H), 1.15 (d, J=6.0 Hz, 3H) ppm.

Step 8: Preparation of tert-butyl ((1S,3r)-3-((S)-1-hydroxyethyl)cyclobutyl)carbamate

To a solution of (((S)-1-((1r,3S)-3-azidocyclobutyl)ethoxy)methyl)benzene (2.84 g, 12.28 mmol, 1 eq) in MeOH (50 mL) were added Boc₂O (4.26 g, 19.52 mmol, 4.48 mL, 1.59 eq) and Pd/C (7.10 g, 6.67 mmol, 10% purity, 0.54 eq) under N₂ atmosphere. The suspension was degassed and backfilled with H₂ for three times and stirred under H₂ atmosphere at 25° C. for 12 hours. After the completion of the reaction, the mixture was filtered through a pad of the Celite, rinsed with methanol (25 mL) and the filtrate was concentrated in vacuo. The desired tert-butyl ((1S,3r)-3-((S)-1-hydroxyethyl)cyclobutyl)carbamate (2.54 g, 11.80 mmol) was obtained as a colorless oil, which was used in the next step without further purification. 1H NMR (400 MHz, CHLOROFORM-d) δ 4.80-4.69 (m, 1H), 4.21-4.13 (m, 1H), 3.84-3.80 (m, 1H), 2.37-2.28 (m, 1H), 2.21-2.15 (m, 2H), 2.06-1.93 (m, 2H), 1.45 (s, 9H), 1.16 (d, J=6.0 Hz, 3H) ppm.

Step 9: Preparation of tert-butyl ((1S,3r)-3-((S)-1-((4-bromopyridin-2-yl)oxy)ethyl)cyclobutyl)carbamate

To a solution of tert-butyl ((1S,3r)-3-((S)-1-hydroxyethyl)cyclobutyl)carbamate (2.54 g, 11.80 mmol, 1.04 eq) and 4-bromo-2-fluoro-pyridine (2 g, 11.36 mmol, 1 eq) in THF (50 mL) at 0° C. was added t-BuOK (2.00 g, 17.82 mmol, 1.57 eq), and the mixture was warmed to 25° C. and stirred for 1 hour under N₂ atmosphere. After the completion of the reaction, the mixture was quenched with saturated aqueous NH₄Cl (50 mL) and extracted with EtOAc (50 mL×2). The combined organic layers were washed with brine (50 mL), dried over anhydrous Na₂SO₄, filtered and the filtrate was concentrated in vacuo. The resulting residue was purified by column chromatography to afford tert-butyl ((1S,3r)-3-((S)-1-((4-bromopyridin-2-yl)oxy)ethyl)cyclobutyl)carbamate (2 g, 40.3% yield) as a colorless oil. LCMS: 371.0, 373.0 [M+H]⁺.

Step 10: Preparation of tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-((tert-butyldimethylsilyl)oxy)cyclopentyl)-1H-pyrazol-5-yl)carbamate

A mixture of tert-butyl ((1S,3r)-3-((S)-1-((4-bromopyridin-2-yl)oxy)ethyl)cyclobutyl)carbamate (2 g, 5.39 mmol, 1.01 eq), 1-(tert-butyl)-3-((1S,3R)-3-((tert-butyldimethylsilyl)oxy)cyclopentyl)-1H-pyrazol-5-amine (1.8 g, 5.33 mmol, 1 eq), Cs₂CO₃ (5.21 g, 16.00 mmol, 3 eq), Xantphos (617.06 mg, 1.07 mmol, 0.2 eq) and Pd₂(dba)₃ (488.27 mg, 0.533 mmol, 0.1 eq) in dioxane (50 mL) was degassed and backfilled with N₂ for three times, and stirred at 90° C. for 12 hours under N₂ atmosphere. After the completion of the reaction, the mixture was cooled to room temperature. The mixture was filtered through a pad of the Celite, rinsed with dioxane (30 mL) and the filtrate was concentrated in vacuo. The resulting residue was purified by column chromatography to afford tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-((tert-butyldimethylsilyl)oxy)cyclopentyl)-1H-pyrazol-5-yl)carbamate (2.75 g, 78.47% yield) as a yellow oil. LCMS: 628.4 [M+H]⁺.

Step 11: Preparation of tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-((tert-butyldimethylsilyl)oxy)cyclopentyl)-1H-pyrazol-5-yl)carbamate (2.75 g, 4.38 mmol, 1 eq) in DCM (50 mL) were added DMAP (109.07 mg, 0.89 mmol, 0.20 eq) and Boc₂O (1.43 g, 6.57 mmol, 1.51 mL, 1.5 eq), and the mixture was stirred at 25° C. for 12 hours under N₂ atmosphere. After the completion of the reaction, the mixture was concentrated in vacuo. The resulting residue was purified by column chromatography to afford tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-((tert-butyldimethylsilyl)oxy)cyclopentyl)-1H-pyrazol-5-yl)carbamate (2.46 g, 76.6% yield) as a yellow oil. LCMS: 728.4 [M+H]⁺.

Step 12: Preparation of tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-hydroxycyclopentyl)-1H-pyrazol-5-yl)carbamate

The solution of tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-((tert-butyldimethylsilyl)oxy)cyclopentyl)-1H-pyrazol-5-yl)carbamate (2.46 g, 3.38 mmol, 1 eq) and TBAF (1 M, 18 mL, 5.33 eq) in THF (50 mL) was stirred at 25° C. for 12 hours under N₂ atmosphere. After the completion of the reaction, the mixture was quenched with H₂O (100 mL) and extracted with EtOAc (50 mL×2). The combined organic layers were washed with brine (100 mL), dried over anhydrous Na₂SO₄, filtered and the filtrate was concentrated in vacuo. The desired tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-hydroxycyclopentyl)-1H-pyrazol-5-yl)carbamate (2.1 g, 3.42 mmol) was obtained as a yellow oil. LCMS: 614.4 [M+H]⁺.

Step 13: Preparation of tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-hydroxycyclopentyl)-1H-pyrazol-5-yl)carbamate (2.07 g, 10.26 mmol, 3 eq), (4-nitrophenyl) carbonochloridate (2.07 g, 10.26 mmol, 3 eq) and DMAP (209 mg, 1.71 mmol, 0.5 eq) in THF (50 mL) was added pyridine (1.76 g, 22.30 mmol, 1.8 mL, 6.52 eq) dropwise. After the completion of the addition, the reaction mixture was stirred at 25° C. for 0.5 hour under N₂ atmosphere. The mixture was then filtered, rinsed with THF (10 mL) and the filtrate was concentrated in vacuo. The resulting residue was purified by column chromatography to afford tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-(((4-nitrophenoxy)carbonyl)oxy)cyclopentyl)-1H-pyrazol-5-yl)carbamate (2.31 g, 2.93 mmol, 85.7% yield) as a colorless oil. LCMS: 779.5 [M+H]⁺.

Step 14: Preparation of (1R,3S)-3-(5-((2-((S)-1-((1r,3S)-3-aminocyclobutyl)ethoxy)pyridin-4-yl)amino)-1-(tert-butyl)-1H-pyrazol-3-yl)cyclopentyl (4-nitrophenyl) carbonate

The solution of tert-butyl (2-((S)-1-((1r,3S)-3-((tert-butoxycarbonyl)amino)cyclobutyl)ethoxy)pyridin-4-yl)(1-(tert-butyl)-3-((1S,3R)-3-(((4-nitrophenoxy)carbonyl)oxy)cyclopentyl)-1H-pyrazol-5-yl)carbamate (2.31 g, 2.97 mmol, 1 eq) and TFA (5 mL) in DCM (50 mL) was stirred at 25° C. for 12 hours under N₂ atmosphere. After the completion of the reaction, the mixture was concentrated in vacuo to afford (1R,3S)-3-(5-((2-((S)-1-((1r,3S)-3-aminocyclobutyl)ethoxy)pyridin-4-yl)amino)-1-(tert-butyl)-1H-pyrazol-3-yl)cyclopentyl (4-nitrophenyl) carbonate (2.05 g, 2.96 mmol) as a yellow oil, which was used into the next step without further purification. LCMS: 579.3 [M+H]⁺.

Step 15: Preparation of (1¹S,1³R,7¹R,7³S,6S,Z)-2¹-(tert-butyl)-6-methyl-2¹H-5,10-dioxa-3,8-diaza-4(4,2)-pyridina-2(3,5)-pyrazola-1(1,3)-cyclopentana-7(1,3)-cyclobutanacyclodecaphan-9-one

To a solution of (1R,3S)-3-(5-((2-((S)-1-((1r,3S)-3-aminocyclobutyl)ethoxy)pyridin-4-yl)amino)-1-(tert-butyl)-1H-pyrazol-3-yl)cyclopentyl (4-nitrophenyl) carbonate (2.05 g, 2.96 mmol, 1 eq) in THF (2.0 L) was added DIEA (11.13 g, 86.12 mmol, 29.10 eq), and the mixture was stirred at 60° C. for 12 hour under N₂ atmosphere. After the completion of the reaction, the mixture was cooled to room temperature, and concentrated in vacuo. The resulting residue was dissolved in EtOAc (300 mL), washed with H₂O (200 mL) and brine (200 mL). The organic layer was dried over anhydrous Na₂SO₄, filtered and the filtrate was concentrated in vacuo. The resulting residue was purified by column chromatography to afford (1¹S,1³R,7¹R,7³S,6S,Z)-2¹-(tert-butyl)-6-methyl-2¹H-5,10-dioxa-3,8-diaza-4(4,2)-pyridina-2(3,5)-pyrazola-1(1,3)-cyclopentana-7(1,3)-cyclobutanacyclodecaphan-9-one (1.4 g, 3.19 mmol) as a yellow solid. LCMS: 440.3 [M+H]⁺.

Step 16: Preparation of (1¹S,1³R,7¹R,7³S,6S,Z)-6-methyl-2¹H-5,10-dioxa-3,8-diaza-4(4,2)-pyridina-2(3,5)-pyrazola-1(1,3)-cyclopentana-7(1,3)-cyclobutanacyclodecaphan-9-one

A mixture of (1¹S,1³R,7¹R,7³S,6S,Z)-2¹-(tert-butyl)-6-methyl-2¹H-5,10-dioxa-3,8-diaza-4(4,2)-pyridina-2(3,5)-pyrazola-1(1,3)-cyclopentana-7(1,3)-cyclobutanacyclodecaphan-9-one (1.4 g, 3.19 mmol, 1 eq) and formic acid (30 mL) was stirred at 100° C. for 2 hours under N₂ atmosphere. After the completion of the reaction, the mixture was cooled to room temperature and concentrated in vacuo. The resulting residue was purified by prep-HPLC {column: Welch Ultimate XB—SiOH 250*50*10 um; mobile phase: [Hexane-EtOH]; gradient:1%-35% B over 15 min} to afford (1¹S,1³R,7¹R,7³S,6S,Z)-6-methyl-2¹H-5,10-dioxa-3,8-diaza-4(4,2)-pyridina-2(3,5)-pyrazola-1(1,3)-cyclopentana-7(1,3)-cyclobutanacyclodecaphan-9-one (783.64 mg, 60.5% yield) as a white solid. LCMS: 384.3 [M+H]⁺; ¹H NMR (400 MHz, DMSO-d₆) δ 12.08 (br s, 1H), 8.91 (s, 1H), 7.75 (d, J=5.6 Hz, 1H), 7.03 (d, J=9.6 Hz, 1H), 6.48 (d, J=1.6 Hz, 1H), 6.45-6.43 (m, 1H), 6.09 (s, 1H), 5.10-5.08 (m, 2H), 4.27-4.21 (m, 1H), 3.23-3.21 (m, 1H), 2.46-2.33 (m, 2H), 2.12-1.99 (m, 3H), 1.96-1.84 (m, 3H), 1.79-1.71 (m, 3H), 1.17 (d, J=6.0 Hz, 3H) ppm.

Example 28: synthesis of (1¹S,1³R,5R,9S,Z)-5,9-dimethyl-2¹H-6,12-dioxa-3,10-diaza-4(4,2)-pyridina-2(3,5)-pyrazola-1(1,3)-cyclopentanacyclododecaphan-11-one (compound 158)

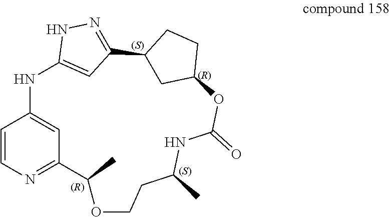

Step 1: Preparation of 1-(4-chloropyridin-2-yl)ethan-1-ol

To a solution of 1-(4-chloropyridin-2-yl)ethan-1-one (5.0 g, 32.14 mmol, 1 eq) in THF (100 mL) was added NaBH₄ (6.1 g, 160.69 mmol, 5 eq), and the mixture was stirred at 20° C. for 2 hours under N₂ atmosphere. After the completion of the reaction, the reaction mixture was concentrated under reduced pressure. The resulting residue was diluted with H₂O (300 mL) and extracted with EtOAc (300 mL×2). The combined organic layers were washed with brine (50 mL), dried over Na₂SO₄, filtered and the filtrate was concentrated under reduced pressure. The resulting residue was purified by column chromatography to give 1-(4-chloropyridin-2-yl)ethan-1-ol (4.3 g, 84.9% yield) as a white solid. LCMS: 158.3 [M+H]⁺.

Step 2: Preparation of (R)-1-(4-chloropyridin-2-yl)ethyl acetate

A mixture of 1-(4-chloropyridin-2-yl)ethan-1-ol (2.0 g, 12.69 mmol, 1 eq), vinyl acetate (3.3 g, 38.07 mmol, 3.5 mL, 3 eq) and Pseudomonas cepacia lipase (2 g) in THF (30 mL) was degassed and purged with N₂ for 3 times, and then the mixture was stirred at 30° C. for 16 hours under N₂ atmosphere. The reaction mixture was then filtered, rinsed with THF (30 mL) and the combined filtrate was concentrated under reduced pressure. The resulting residue was purified by column chromatography to give (R)-1-(4-chloropyridin-2-yl)ethyl acetate (320.0 mg, 12.4% yield) as a white solid. LCMS: 200.0 [M+H]⁺.

Step 3: Preparation of (R)-1-(4-chloropyridin-2-yl)ethan-1-ol

To a solution of (R)-1-(4-chloropyridin-2-yl)ethyl acetate (300.0 mg, 1.50 mmol, 1 eq) in MeOH (5 mL) was added K₂CO₃ (311.5 mg, 2.25 mmol, 1.5 eq), and the mixture was stirred at 20° C. for 2 hours under N₂ atmosphere. After the completion of the reaction, the reaction mixture was diluted with water (20 mL) and extracted with EA (30 mL×2). The combined organic layers were washed with water (30 mL), dried over Na₂SO₄, filtered, and the filtrate was concentrated under reduced pressure. The desired (R)-1-(4-chloropyridin-2-yl)ethan-1-ol (220 mg, 1.40 mmol, 92.9% yield) was obtained as a colorless oil, which was used for the next step without further purification.

Step 4: Preparation of tert-butyl ((S)-4-((R)-1-(4-chloropyridin-2-yl)ethoxy)butan-2-yl)carbamate

To a solution of (1R)-1-(4-chloro-2-pyridyl)ethanol (140.0 mg, 0.89 mmol, 1 eq) in THF (3 mL) was added NaH (71.1 mg, 1.78 mmol, 60% purity, 2 eq) at 0° C., followed by tert-butyl N-[(1S)-3-iodo-1-methyl-propyl]carbamate (398.62 mg, 1.33 mmol, 1.5 eq), and the mixture was warmed to 25° C. and stirred at for 2 hours under N₂ atmosphere. After the completion of the reaction, the reaction mixture was quenched with saturated aqueous NH₄Cl (30 mL) and extracted by EtOAc (50 mL×3). The organic phase was washed with brine (100 mL), dried over anhydrous Na₂SO₄, filtered and the filtrate was concentrated in vacuum. The resulting residue was purified by column chromatography to give tert-butyl ((S)-4-((R)-1-(4-chloropyridin-2-yl)ethoxy)butan-2-yl)carbamate (100.0 mg, 0.3 mmol, 34.2% yield) as an off-white oil. LCMS: 329.1 [M+H]⁺, 273.1 [M−55+H]⁺.

Step 5: Preparation of tert-butyl ((S)-4-((R)-1-(4-((1-(tert-butyl)-3-((1S,3R)-3-((tert-butyldimethylsilyl)oxy)cyclopentyl)-1H-pyrazol-5-yl)amino)pyridin-2-yl)ethoxy)butan-2-yl)carbamate

A mixture of tert-butyl ((S)-4-((R)-1-(4-chloropyridin-2-yl)ethoxy)butan-2-yl)carbamate (100.0 mg, 0.3 mmol, 1 eq), 1-(tert-butyl)-3-((1S,3R)-3-((tert-butyldimethylsilyl)oxy)cyclopentyl)-1H-pyrazol-5-amine (112.9 mg, 0.33 mmol, 1.1 eq), Pd₂(dba)₃ (27.8 mg, 0.03 mmol, 0.1 eq), Xantphos (35.2 mg, 0.06 mmol, 0.2 eq) and Cs₂CO₃ (198.2 mg, 0.61 mmol, 2 eq) in dioxane (2 mL) was degassed and backfilled with N₂ for 3 times, and stirred at 90° C. for 12 hours under N₂ atmosphere. After the completion of the reaction, the mixture was cooled to room temperature. The reaction mixture was filtered through a short pad of celite, rinsed with dioxane (15 mL), and the filtrate was concentrated under reduced pressure. The resulting residue was purified by column chromatography to give tert-butyl ((S)-4-((R)-1-(4-((1-(tert-butyl)-3-((1S,3R)-3-((tert-butyldimethylsilyl)oxy)cyclopentyl)-1H-pyrazol-5-yl)amino)pyridin-2-yl)ethoxy)butan-2-yl)carbamate (150 mg, 0.24 mmol, 78.3% yield) as a yellow oil. LCMS: 630.6 [M+H]⁺.

Step 6: Preparation of tert-butyl (2-((R)-1-((S)-3-((tert-butoxycarbonyl)amino)butoxy)ethyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-4-((R)-1-(4-((1-(tert-butyl)-3-((1S,3R)-3-((tert-butyldimethylsilyl)oxy)cyclopentyl)-1H-pyrazol-5-yl)amino)pyridin-2-yl)ethoxy)butan-2-yl)carbamate (150.0 mg, 0.24 mmol, 1 eq) in DCM (3 mL) was added Boc₂O (103.9 mg, 0.48 mmol, 0.11 mL, 2 eq) and DMAP (29.1 mg, 0.24 mmol, 1 eq), and the mixture was stirred at 25° C. for 1 hour under N₂ atmosphere. After the completion of the reaction, the reaction mixture was diluted with water (30 mL) and extracted by dichloromethane (30 mL×3). The combined organic phase was washed with brine (50 mL), dried over anhydrous Na₂SO₄, filtered and the filtrate was concentrated in vacuum. The resulting residue was purified by column chromatography to give tert-butyl (2-((R)-1-((S)-3-((tert-butoxycarbonyl)amino)butoxy)ethyl)pyridin-4-yl)(1-(tert-butyl)-3-((1S,3R)-3-((tert-butyldimethylsilyl)oxy)cyclopentyl)-1H-pyrazol-5-yl)carbamate (150 mg, 86.3% yield) as a yellow oil. LCMS: 730.5 [M+H]⁺.

Step 7: Preparation of tert-butyl (2-((R)-1-((S)-3-((tert-butoxycarbonyl)amino)butoxy)ethyl)pyridin-4-yl)(1-(tert-butyl)-3-((1S,3R)-3-hydroxycyclopentyl)-1H-pyrazol-5-yl)carbamate

The solution of tert-butyl (2-((R)-1-((S)-3-((tert-butoxycarbonyl)amino)butoxy)ethyl)pyridin-4-yl)(1-(tert-butyl)-3-((1S,3R)-3-((tert-butyldimethylsilyl)oxy)cyclopentyl)-1H-pyrazol-5-yl)carbamate (150.0 mg, 0.21 mmol, 1 eq) and TBAF (1 M, 2 mL, 10 eq) in THF (2 mL) was stirred at 25° C. for 12 hours under N₂ atmosphere. After the completion of the reaction, the reaction mixture was diluted with water (30 mL) and extracted with EtOAc (30 mL×3). The combined organic phase was washed with brine (10 mL), dried over anhydrous Na₂SO₄, filtered and the filtrate was concentrated in vacuum. The desired tert-butyl (2-((R)-1-((S)-3-((tert-butoxycarbonyl)amino)butoxy)ethyl)pyridin-4-yl)(1-(tert-butyl)-3-((1S,3R)-3-hydroxycyclopentyl)-1H-pyrazol-5-yl)carbamate (120 mg, 0.21 mmol) was obtained as an off-white gum, which was used in the next step without further purification. LCMS: 616.4 [M+H]⁺.

Step 8: Preparation of tert-butyl (2-((R)-1-((S)-3-((tert-butoxycarbonyl)amino)butoxy)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((R)-1-((S)-3-((tert-butoxycarbonyl)amino)butoxy)ethyl)pyridin-4-yl)(1-(tert-butyl)-3-((1S,3R)-3-hydroxycyclopentyl)-1H-pyrazol-5-yl)carbamate (120 mg, 0.19 mmol, 1 eq) and (4-nitrophenyl) carbonochloridate (117.8 mg, 0.59 mmol, 3 eq) in DCM (2 mL) was added Pyridine (490.0 mg, 6.19 mmol, 32 eq) and DMAP (23.8 mg, 0.19 mmol, 1 eq), and the mixture was stirred at 25° C. for 2 hours under N₂ atmosphere. After the completion of the reaction, the reaction mixture was quenched with water (30 mL) and extracted with dichloromethane (30 mL×3). The combined organic phase was washed with brine (30 mL), dried over anhydrous Na₂SO₄, filtered and the filtrate was concentrated in vacuum.

The resulting residue was purified by column chromatography to give tert-butyl (2-((R)-1-((S)-3-((tert-butoxycarbonyl)amino)butoxy)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate (150 mg, 98.6% yield) as a yellow oil. LCMS: 781.3 [M+H]⁺.

Step 9: Preparation of (1R,3S)-3-(5-((2-((R)-1-((S)-3-aminobutoxy)ethyl)pyridin-4-yl)amino)-1-(tert-butyl)-1H-pyrazol-3-yl)cyclopentyl (4-nitrophenyl) carbonate

The solution of tert-butyl (2-((R)-1-((S)-3-((tert-butoxycarbonyl)amino)butoxy)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate (150.0 mg, 0.19 mmol, 1 eq) and TFA (1.5 g, 13.46 mmol, 70 eq) in DCM (2 mL) was stirred at 25° C. for 8 hours under N₂ atmosphere. After the completion of the reaction, the volatiles were removed under reduced pressure. The desired (1R,3S)-3-(5-((2-((R)-1-((S)-3-aminobutoxy)ethyl)pyridin-4-yl)amino)-1-(tert-butyl)-1H-pyrazol-3-yl)cyclopentyl (4-nitrophenyl) carbonate (100 mg, 0.17 mmol) was obtained as a yellow oil, which was used in the next step without further purification. LCMS: 581.4 [M+H]⁺.

Step 10: Preparation of (1¹S,1³R,5R,9S,Z)-2¹-(tert-butyl)-5,9-dimethyl-2¹H-6,12-dioxa-3,10-diaza-4(4,2)-pyridina-2(3,5)-pyrazola-1(1,3)-cyclopentanacyclododecaphan-11-one

To a solution of (1R,3S)-3-(5-((2-((R)-1-((S)-3-aminobutoxy)ethyl)pyridin-4-yl)amino)-1-(tert-butyl)-1H-pyrazol-3-yl)cyclopentyl (4-nitrophenyl) carbonate (100 mg, 0.17 mmol, 1 eq) in THF (200 mL) was added DIEA (742.0 mg, 5.74 mmol, 33 eq), and the mixture was stirred at 25° C. for 12 hours under N₂ atmosphere. After the completion of the reaction, the reaction mixture was concentrated under reduced pressure to give (1¹S,1³R,5R,9S,Z)-2¹-(tert-butyl)-5,9-dimethyl-2¹H-6,12-dioxa-3,10-diaza-4(4,2)-pyridina-2(3,5)-pyrazola-1(1,3)-cyclopentanacyclododecaphan-11-one (70 mg, 0.16 mmol) as a yellow oil, which was used in the next step without further purification. LCMS: 442.4 [M+H]⁺.

Step 11: Preparation of (1¹S,1³R,5R,9S,Z)-5,9-dimethyl-2¹H-6,12-dioxa-3,10-diaza-4(4,2)-pyridina-2(3,5)-pyrazola-1(1,3)-cyclopentanacyclododecaphan-11-one

The solution of (1¹S,1³R,5R,9S,Z)-2¹-(tert-butyl)-5,9-dimethyl-2¹H-6,12-dioxa-3,10-diaza-4(4,2)-pyridina-2(3,5)-pyrazola-1(1,3)-cyclopentanacyclododecaphan-11-one (70.0 mg, 0.16 mmol, 1 eq) in formic acid (2 mL) was stirred at 100° C. for 2 hours under N₂ atmosphere. After the completion of the reaction, the mixture was cooled to room temperature and the volatiles were removed under reduced pressure. The resulting residue was purified by prep-HPLC (column: Phenomenex Luna C18 150×25 mm×10 um; mobile phase: [water (FA)-ACN]; gradient:1%-30% B over 10 min) to give (1¹S,1³R,5R,9S,Z)-5,9-dimethyl-2¹H-6,12-dioxa-3,10-diaza-4(4,2)-pyridina-2(3,5)-pyrazola-1(1,3)-cyclopentanacyclododecaphan-11-one (35.7 mg, 58.3% yield) as an off-white solid. LCMS: 386.2 [M+H]⁺; ¹H NMR (400 MHz, DMSO-d₆) δ 8.97 (s, 1H), 8.14 (s, 1H), 8.03 (d, J=5.6 Hz, 1H), 7.01 (d, J=7.6 Hz, 1H), 6.82 (s, 1H), 6.67 (dd, J=2.0, J=5.6 Hz, 1H), 5.91 (s, 1H), 4.96 (s, 1H), 4.34 (q, J=6.4 Hz, 1H), 3.73-3.61 (m, 3H), 3.20 (d, J=8.0 Hz, 2H), 3.12-3.06 (m, 1H), 2.39 (s, 1H), 2.13-1.99 (m, 1H), 1.84-1.73 (m, 4H), 1.62-1.48 (m, 1H), 1.23 (dd, J=2.4, J=6.4 Hz, 3H), 0.99 (d, J=6.4 Hz, 3H) ppm.

Example 29: synthesis of (1¹S,1³R,5S,9S,Z)-5,9-dimethyl-2¹H-6,12-dioxa-3,10-diaza-4(4,2)-pyridina-2(3,5)-pyrazola-1(1,3)-cyclopentanacyclododecaphan-11-one (compound 159)

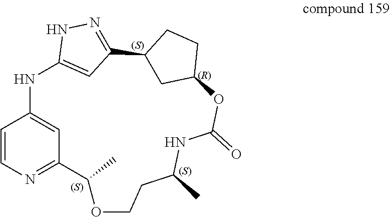

Step 1: Preparation of (S)-1-(4-chloropyridin-2-yl)ethyl 4-nitrobenzoate

To a solution of (R)-1-(4-chloropyridin-2-yl)ethan-1-ol (300 mg, 1.90 mmol, 1 eq), 4-nitrobenzoic acid (381.75 mg, 2.28 mmol, 1.2 eq) and PPh₃ (599.14 mg, 2.28 mmol, 1.2 eq) in THF (4 mL) was added dropwise DIAD (461.90 mg, 2.28 mmol, 0.44 ml, 1.2 eq) at 0° C. under N₂ atmosphere. After the completion of addition, the mixture was warmed to 25° C. and stirred for 4 hours under N₂ atmosphere. The mixture was then diluted with H₂O (100 mL) and extracted with ethyl acetate (50 mL×3). The combined organic layers were washed with brine (50 mL×2), dried over Na₂SO₄, filtered, and the filtrate was concentrated under reduced pressure. The resulting residue was purified by column chromatography to afford (S)-1-(4-chloropyridin-2-yl)ethyl 4-nitrobenzoate (520 mg, 89.1% yield) as a colorless oil. LCMS: 307.1 [M+H]⁺.

Step 2: Preparation of (S)-1-(4-chloropyridin-2-yl)ethan-1-ol

A mixture of (S)-1-(4-chloropyridin-2-yl)ethyl 4-nitrobenzoate (520 mg, 1.70 mmol, 1 eq) and LiOH (142.30 mg, 3.39 mmol, 2 eq) in THF (4 mL) and H₂O (4 mL) was stirred at 25° C. for 4 hours under N₂ atmosphere. After the completion of the reaction, the reaction mixture was diluted with H₂O (50 mL) and extracted with ethyl acetate (50 mL×3). The combined organic layers were washed with brine (50 mL), dried over Na₂SO₄, filtered and the filtrate was concentrated under reduced pressure. The desired (S)-1-(4-chloropyridin-2-yl)ethan-1-ol (210 mg, 72.3% yield) was used in the next step without further purification. LCMS: 158.4 [M+H]⁺.

Step 3: Preparation of tert-butyl ((S)-4-((S)-1-(4-chloropyridin-2-yl)ethoxy)butan-2-yl)carbamate

A mixture of (S)-1-(4-chloropyridin-2-yl)ethan-1-ol (210 mg, 1.33 mmol, 1 eq), tert-butyl (S)-(4-iodobutan-2-yl)carbamate (597.93 mg, 2.00 mmol, 1.5 eq) and Ag₂O (926.37 mg, 4.00 mmol, 3 eq) in acetonitrile (8 mL) was stirred at 80° C. for 16 hours under N₂ atmosphere. After the completion of the reaction, the mixture was cooled to room temperature and the solid was filtered off, rinsed with acetonitrile (10 mL). The filtrate was concentrated under reduced pressure. The resulting residue was purified by column chromatography to afford tert-butyl ((S)-4-((S)-1-(4-chloropyridin-2-yl)ethoxy)butan-2-yl)carbamate (100 mg, 22.8% yield) as a yellow solid. LCMS: 329.2 [M+H]⁺.

Step 4: Preparation of afford tert-butyl ((S)-4-((S)-1-(4-((1-(tert-butyl)-3-((1S,3R)-3-((tert-butyldimethylsilyl)oxy)cyclopentyl)-1H-pyrazol-5-yl)amino)pyridin-2-yl)ethoxy)butan-2-yl)carbamate

A mixture of tert-butyl ((S)-4-((S)-1-(4-chloropyridin-2-yl)ethoxy)butan-2-yl)carbamate (95 mg, 0.29 mmol, 1 eq), 1-(tert-butyl)-3-((1S,3R)-3-((tert-butyldimethylsilyl)oxy)cyclopentyl)-1H-pyrazol-5-amine (97.53 mg, 0.29 mmol, 1 eq), Cs₂CO₃ (188.26 mg, 0.58 mmol, 2 eq), Xantphos (33.43 mg, 0.058 mmol, 0.2 eq) and Pd₂(dba)₃ (52.91 mg, 0.058 mmol, 0.2 eq) in dioxane (3 mL) was degassed and backfilled with N₂ for 3 times, and was stirred at 90° C. for 12 hours under N₂ atmosphere. After the completion of the reaction, the mixture was cooled to room temperature. The reaction mixture was filtered through a short pad of celite, rinsed with dioxane (10 mL) and the filtrate was concentrated under reduced pressure. The resulting residue was purified by column chromatography to afford tert-butyl ((S)-4-((S)-1-(4-((1-(tert-butyl)-3-((1S,3R)-3-((tert-butyldimethylsilyl)oxy)cyclopentyl)-1H-pyrazol-5-yl)amino)pyridin-2-yl)ethoxy)butan-2-yl)carbamate (103 mg, 0.16351 mmol, 56.6% yield) as a yellow oil. LCMS: 630.6 [M+H]⁺.

Step 5: Preparation of tert-butyl (2-((S)-1-((S)-3-((tert-butoxycarbonyl)amino)butoxy)ethyl)pyridin-4-yl)(1-(tert-butyl)-3-((1S,3R)-3-((tert-butyldimethylsilyl)oxy)cyclopentyl)-1H-pyrazol-5-yl)carbamate

A mixture of tert-butyl ((S)-4-((S)-1-(4-((1-(tert-butyl)-3-((1S,3R)-3-((tert-butyldimethylsilyl)oxy)cyclopentyl)-1H-pyrazol-5-yl)amino)pyridin-2-yl)ethoxy)butan-2-yl)carbamate (98 mg, 0.16 mmol, 1 eq), Boc₂O (67.90 mg, 0.31 mmol, 0.071 ml, 2 eq) and DMAP (19.01 mg, 0.16 mmol, 1 eq) in DCM (0.5 mL) was stirred at 25° C. for 4 hours under N₂ atmosphere. After the completion of the reaction, the volatiles were removed under reduced pressure. The resulting residue was purified by column chromatography to afford tert-butyl (2-((S)-1-((S)-3-((tert-butoxycarbonyl)amino)butoxy)ethyl)pyridin-4-yl)(1-(tert-butyl)-3-((1S,3R)-3-((tert-butyldimethylsilyl)oxy)cyclopentyl)-1H-pyrazol-5-yl)carbamate (71 mg, 62.5% yield) as a yellow oil. LCMS: 730.7 [M+H]⁺.

Step 6: Preparation of tert-butyl (2-((S)-1-((S)-3-((tert-butoxycarbonyl)amino)butoxy)ethyl)pyridin-4-yl)(1-(tert-butyl)-3-((1S,3R)-3-hydroxycyclopentyl)-1H-pyrazol-5-yl)carbamate

A mixture of tert-butyl (2-((S)-1-((S)-3-((tert-butoxycarbonyl)amino)butoxy)ethyl)pyridin-4-yl)(1-(tert-butyl)-3-((1S,3R)-3-((tert-butyldimethylsilyl)oxy)cyclopentyl)-1H-pyrazol-5-yl)carbamate (71 mg, 0.09725 mmol, 1 eq) and TBAF (1 M, 0.58351 ml, 6 eq) in THF (0.5 mL) was stirred at 25° C. for 16 hours under N₂ atmosphere. After the completion of the reaction, the reaction mixture was diluted with H₂O (50 mL) and extracted with ethyl acetate (50 mL×3). The combined organic layers were washed with brine (50 mL), dried over Na₂SO₄, filtered and the filtrate was concentrated under reduced pressure. The desired tert-butyl (2-((S)-1-((S)-3-((tert-butoxycarbonyl)amino)butoxy)ethyl)pyridin-4-yl)(1-(tert-butyl)-3-((1S,3R)-3-hydroxycyclopentyl)-1H-pyrazol-5-yl)carbamate (52 mg, 0.084 mmol, 86.8% yield) was obtained as a brown oil, which was used in the next step without further purification. LCMS: 616.4 [M+H]⁺.

Step 7: Preparation of tert-butyl (2-((S)-1-((S)-3-((tert-butoxycarbonyl)amino)butoxy)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((S)-1-((S)-3-((tert-butoxycarbonyl)amino)butoxy)ethyl)pyridin-4-yl)(1-(tert-butyl)-3-((1S,3R)-3-hydroxycyclopentyl)-1H-pyrazol-5-yl)carbamate (47 mg, 0.076 mmol, 1 eq) in DCM (2 mL) was added DMAP (9.32 mg, 0.076 mmol, 1 eq), (4-nitrophenyl) carbonochloridate (30.77 mg, 0.15 mmol, 2 eq) and pyridine (36.22 mg, 0.46 mmol, 0.037 ml, 6 eq), and the mixture was stirred at 25° C. for 2 hours under N₂ atmosphere. After the completion of the reaction, the reaction mixture was diluted with H₂O (10 mL) and extracted with ethyl acetate (10 mL×3). The combined organic layers were washed with brine (5 mL), dried over Na₂SO₄, filtered and the filtrate was concentrated under reduced pressure. The resulting residue was purified by column chromatography to afford tert-butyl (2-((S)-1-((S)-3-((tert-butoxycarbonyl)amino)butoxy)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate (50 mg, 83.9% yield) as a yellow oil. LCMS: 781.5 [M+H]⁺.

Step 8: Preparation of (1R,3S)-3-(5-((2-((S)-1-((S)-3-aminobutoxy)ethyl)pyridin-4-yl)amino)-1-(tert-butyl)-1H-pyrazol-3-yl)cyclopentyl (4-nitrophenyl) carbonate

The solution of tert-butyl (2-((S)-1-((S)-3-((tert-butoxycarbonyl)amino)butoxy)ethyl)pyridin-4-yl)(1-(tert-butyl)-3-((1S,3R)-3-(((4-nitrophenoxy)carbonyl)oxy)cyclopentyl)-1H-pyrazol-5-yl)carbamate (50 mg, 0.064 mmol, 1 eq) and TFA (767.50 mg, 6.73 mmol, 0.5 mL, 105.1 eq) in DCM (1 mL) was stirred at 25° C. for 12 hours under N₂ atmosphere. After the completion of the reaction, the reaction mixture was concentrated under reduced pressure. The desired (1R,3S)-3-(5-((2-((S)-1-((S)-3-aminobutoxy)ethyl)pyridin-4-yl)amino)-1-(tert-butyl)-1H-pyrazol-3-yl)cyclopentyl (4-nitrophenyl) carbonate (32 mg, 86.1% yield) was obtained as a brown oil, which was used in the next step without further purification. LCMS: 581.5 [M+H]⁺.

Step 9: Preparation of (1¹S,1³R,5S,9S,Z)-2¹-(tert-butyl)-5,9-dimethyl-2¹H-6,12-dioxa-3,10-diaza-4(4,2)-pyridina-2(3,5)-pyrazola-1(1,3)-cyclopentanacyclododecaphan-11-one

To a solution of (1R,3S)-3-(5-((2-((S)-1-((S)-3-aminobutoxy)ethyl)pyridin-4-yl)amino)-1-(tert-butyl)-1H-pyrazol-3-yl)cyclopentyl (4-nitrophenyl) carbonate (32 mg, 0.055 mmol, 1 eq) in THF (100 mL) was added DIEA (42.73 mg, 0.33 mmol, 0.057 ml, 6 eq), and the mixture was stirred at 25° C. for 12 hours under N₂ atmosphere. After the completion of the reaction, the reaction mixture was concentrated under reduced pressure. The desired (1¹S,1³R,5S,9S,Z)-2¹-(tert-butyl)-5,9-dimethyl-2¹H-6,12-dioxa-3,10-diaza-4(4,2)-pyridina-2(3,5)-pyrazola-1(1,3)-cyclopentanacyclododecaphan-11-one (21 mg, 0.047.56 mmol) was obtained as a brown oil, which was used in the next step without further purification. LCMS: 442.3 [M+H]⁺.

Step 10: Preparation of (1¹S,1³R,5S,9S,Z)-5,9-dimethyl-2¹H-6,12-dioxa-3,10-diaza-4(4,2)-pyridina-2(3,5)-pyrazola-1(1,3)-cyclopentanacyclododecaphan-11-one

A solution of (1¹S,1³R,5S,9S,Z)-2¹-(tert-butyl)-5,9-dimethyl-2¹H-6,12-dioxa-3,10-diaza-4(4,2)-pyridina-2(3,5)-pyrazola-1(1,3)-cyclopentanacyclododecaphan-11-one (21 mg, 0.047 mmol, 1 eq) in formic acid (1.5 mL) was stirred at 100° C. for 4 hours under N₂ atmosphere. After the completion of the reaction, the mixture was cooled to room temperature and the volatiles was removed under reduced pressure. The resulting residue was purified by prep-HPLC (column: Phenomenex luna C18 150*25 mm*10 um; mobile phase: [water (FA)-ACN]; gradient:5%-35% B over 10 min) to afford (1¹S,1³R,5S,9S,Z)-5,9-dimethyl-2¹H-6,12-dioxa-3,10-diaza-4(4,2)-pyridina-2(3,5)-pyrazola-1 (1,3)-cyclopentanacyclododecaphan-11-one (13.39 mg, 0.034 mmol, 72.6% yield, 99.4% purity) as a white solid. LCMS: 386.2 [M+H]⁺; ¹H NMR (400 MHz, DMSO-d₆) δ 12.79-11.98 (br, 1H), 9.27-9.20 (br, 1H), 8.00-7.97 (m, 1H), 7.00 (s, 1H), 6.76-6.66 (m, 2H), 5.98 (s, 1H), 5.18-5.15 (m, 1H), 4.29-4.26 (m, 1H), 3.66-3.64 (m, 2H), 3.47-3.42 (m, 2H), 2.47-2.40 (m, 1H), 2.29-2.16 (m, 1H), 1.96-1.94 (m, 1H), 1.83-1.78 (m, 4H), 1.28-1.26 (m, 1H), 1.30-1.25 (m, 3H), 1.01-0.95 (m, 1H), 0.90-0.79 (m, 2H) ppm.

Example 30: synthesis of (1¹S,1³R,6S,9S,Z)-6,9-dimethyl-2¹H-5,12-dioxa-3,10-diaza-4(4,2)-pyridina-2(3,5)-pyrazola-1(1,3)-cyclopentanacyclododecaphan-11-one (compound 162)

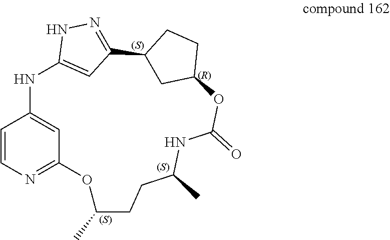

Step 1: Preparation of (S)-4-bromo-2-(pent-4-en-2-yloxy)pyridine

A mixture of 4-bromopyridin-2-ol (4 g, 22.99 mmol, 1 eq), (R)-pent-4-en-2-ol (2.2 g, 25.29 mmol, 1.1 eq), PPh₃ (7.2 g, 27.59 mmol, 1.2 eq) and DIAD (5.6 g, 27.59 mmol, 5.35 mL, 1.2 eq) in THF (80 mL) was stirred at 25° C. for 2 hours under N₂ atmosphere. The mixture was then quenched with H₂O (200 mL) and extracted with EtOAc (200 mL×2). The combined organic layers were washed with brine (100 mL×2), dried over Na₂SO₄, filtered and the filtrate was concentrated in vacuo. The resulting residue was purified by column chromatography to afford (S)-4-bromo-2-(pent-4-en-2-yloxy)pyridine (3.6 g, 64.7% yield) as a colorless oil. LCMS: 242.0, 244.0 [M+H]⁺.

Step 2: Preparation of (S)-4-((4-bromopyridin-2-yl)oxy)pentan-1-ol

To a solution of (S)-4-bromo-2-(pent-4-en-2-yloxy)pyridine (2.6 g, 10.74 mmol, 1 eq) in THF (40 mL) was added BH₃·THF (1 M, 21.48 mL, 2 eq) dropwise at 0° C.. After the completion of the addition, the mixture was warmed to 25° C. and stirred for an additional 3 hours under N₂ atmosphere. The mixture was then quenched with MeOH (1.7 g, 53.69 mmol, 2.17 mL, 5 eq) at 0° C., followed by the addition of H₂O₂ (5 g, 32.22 mmol, 6.2 mL, 3 eq), and warmed to 25° C. and stirred for 12 hours at that temperature. After the completion of the reaction, the mixture was quenched with saturated aqueous Na₂SO₃ (50 mL) and extracted with EtOAc (50 mL×3). The combined organic layers were washed with brine (100 mL), dried over anhydrous Na₂SO₄, filtered and the filtrate was concentrated in vacuo. The resulting residue was purified by column chromatography to afford (S)-4-((4-bromopyridin-2-yl)oxy)pentan-1-ol (1.6 g, 57.3% yield) as a colorless oil. LCMS: 260.0, 263.0 [M+H]⁺, ¹H NMR (400 MHz, CHLOROFORM-d) δ 7.95 (d, J=5.50 Hz, 1H), 7.00-6.98 (dd, J=5.50, 1.63 Hz, 1H), 6.91 (1 d, J=1.50 Hz, 1H), 5.23-5.18 (m, 1H), 3.72-3.68 (m, 2H), 1.65-1.82 (m, 4H), 1.32 (d, J=6.25 Hz, 3H) ppm.

Step 3: Preparation of (S)-4-((4-bromopyridin-2-yl)oxy)pentanal

To a solution of (S)-4-((4-bromopyridin-2-yl)oxy)pentan-1-ol (1.4 g, 5.38 mmol, 1 eq) in DCM (30 mL) was added DMP (2.3 g, 5.49 mmol, 1.70 mL, 1 eq) at 0° C., and the mixture was warmed to 25° C. and stirred for 12 hours at that temperature under N₂ atmosphere. After the completion of the reaction, the mixture was poured into H₂O (150 mL), extracted with EtOAc (200 mL×2). The combined organic layers were washed with brine (100 mL), dried over anhydrous Na₂SO₄, filtered and the filtrate was concentrated to give (S)-4-((4-bromopyridin-2-yl)oxy)pentanal (1.3 g, 5.04 mmol) as a colorless oil. LCMS: 258.1, 260.1 [M+H]⁺.

Step 4: Preparation of (R)—N—((S,E)-4-((5-bromopyridin-3-yl)oxy)pentylidene)-2-methylpropane-2-sulfinamide

To a solution of (S)-4-((4-bromopyridin-2-yl)oxy)pentanal (600 mg, 2.32 mmol, 1 eq) and (R)-2-methylpropane-2-sulfinamide (338 mg, 2.79 mmol, 1.2 eq) in THF (5 mL) was added CuSO₄ (742 mg, 4.65 mmol, 2 eq) and molecular sieves (5 g), and the mixture was stirred at 60° C. for 3 hours under N₂ atmosphere. After the completion of the reaction, the mixture was cooled to room temperature. The mixture was filtered through a pad of the Celite, rinsed with THF (10 mL), and the filtrate was concentrated under reduced pressure. The resulting residue was purified by column chromatography to afford (R)—N—((S,E)-4-((5-bromopyridin-3-yl)oxy)pentylidene)-2-methylpropane-2-sulfinamide (570 mg, 67.8% yield) as a light yellow oil. LCMS: 361.1, 363.1 [M+H]⁺.

Step 5: Preparation of (R)—N-((2S,5S)-5-((4-bromopyridin-2-yl)oxy)hexan-2-yl)-2-methylpropane-2-sulfinamide

To a solution of (R)—N—((S,E)-4-((5-bromopyridin-3-yl)oxy)pentylidene)-2-methylpropane-2-sulfinamide (570 mg, 1.58 mmol, 1 eq) in THF (10 mL) was added MeMgBr (3 M, 1.58 mL, 3 eq) dropwise at 0° C. under N₂ atmosphere. After the completion of the addition, the mixture was warmed to 50° C. and stirred for 2 hours under N₂ atmosphere. After the completion of the reaction, the mixture was cooled to room temperature, quenched with saturated aqueous NH₄Cl (100 mL) and extracted with EtOAc (100 mL×3). The combined organic layers were washed with brine (100 mL), dried over anhydrous Na₂SO₄, filtered and the filtrate was concentrated in vacuo. The resulting residue was purified by column chromatography to afford (R)—N-((2S,5S)-5-((4-bromopyridin-2-yl)oxy)hexan-2-yl)-2-methylpropane-2-sulfinamide (390 mg, 65.5% yield) as a light-yellow oil. LCMS: 377.1, 379.1 [M+H]⁺.

Step 6: Preparation of (2S,5S)-5-((4-bromopyridin-2-yl)oxy)hexan-2-amine

To a solution of (R)—N-((2S,5S)-5-((4-bromopyridin-2-yl)oxy)hexan-2-yl)-2-methylpropane-2-sulfinamide (390 mg, 1.03 mmol, 1 eq) in dioxane (6 mL) was added a solution of HCl in dioxane (4 M, 7.80 mL, 30 eq), and the mixture was stirred at 25° C. for 1 hour. After the completion of the reaction, the mixture was then concentrated in vacuo to afford (2S,5S)-5-((4-bromopyridin-2-yl)oxy)hexan-2-amine (300 mg, 1.10 mmol) as a light-yellow oil, which was used in the next step without further purification. LCMS: 273.1, 275.1 [M+H]⁺.

Step 7: Preparation of tert-butyl ((2S,5S)-5-((4-bromopyridin-2-yl)oxy)hexan-2-yl)carbamate

To a solution of (2S,5S)-5-((4-bromopyridin-2-yl)oxy)hexan-2-amine (390 mg, 1.43 mmol, 1 eq) and TEA (1.5 g, 14.37 mmol, 2 mL, 10 eq) in DCM (8 mL) was added (Boc)₂O (467.4 mg, 2.14 mmol, 1.5 eq), and the mixture was stirred at 25° C. for 1 hour. After the completion of the reaction, the reaction mixture was concentrated under reduced pressure. The resulting residue was purified by column chromatography to afford tert-butyl ((2S,5S)-5-((4-bromopyridin-2-yl)oxy)hexan-2-yl)carbamate (460 mg, 86.3% yield) as a colorless oil. LCMS: 373.1, 375.1 [M+H]⁺.

Step 8: Preparation of tert-butyl ((2S,5S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)hexan-2-yl)carbamate

A mixture of (2S,5S)-5-((4-bromopyridin-2-yl)oxy)hexan-2-amine (250 mg, 0.670 mmol, 1 eq), 1-(tert-butyl)-3-((1S,3R)-3-((tert-butyldimethylsilyl)oxy)cyclopentyl)-1H-pyrazol-5-amine (226 mg, 0.670 mmol, 1 eq), Pd₂(dba)₃ (61.3 mg, 0.067 mmol, 0.1 eq), Xantphos (58.1 mg, 0.01 mol, 0.15 eq) and Cs₂CO₃ (436.4 mg, 1.34 mmol, 2 eq) in dioxane (4 mL) was degassed and backfilled with N₂ for 3 times, and then stirred at 90° C. for 16 hours under N₂ atmosphere. After the completion of the reaction, the mixture was cooled to room temperature. The reaction mixture was filtered through a short pad of celite, rinsed with dioxane (5 mL), and concentrated under reduced pressure. The resulting residue was purified by column chromatography to afford tert-butyl ((2S,5S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)hexan-2-yl)carbamate (400 mg, 94.8% yield) as a colorless oil. LCMS: 630.6 [M+H]⁺.

Step 9: Preparation of tert-butyl (2-(((2S,5S)-5-((tert-butoxycarbonyl)amino)hexan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((2S,5S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)hexan-2-yl)carbamate (380 mg, 0.603 mmol, 1 eq) in DCM (5 mL) was added Boc₂O (197.5 mg, 0.905 mmol, 1.5 eq) and DMAP (73.69 mg, 0.603 mmol, 1 eq), and the mixture was stirred at 20° C. for 1 hour. After the completion of the reaction, the mixture was filtered, rinsed with DCM (5 mL) and the filtrate was concentrated in vacuo. The resulting residue was purified by column chromatography to afford tert-butyl (2-(((2S,5S)-5-((tert-butoxycarbonyl)amino)hexan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate (290 mg, 0.397 mmol, 65.9% yield) as a colorless oil. LCMS: 730.6 [M+H]⁺.

Step 10: Preparation of tert-butyl (2-(((2S,5S)-5-((tert-butoxycarbonyl)amino)hexan-2-yl)oxy)pyridin-4-yl)(1-(tert-butyl)-3-((1S,3R)-3-hydroxycyclopentyl)-1H-pyrazol-5-yl)carbamate

To a solution of tert-butyl (2-(((2S,5S)-5-((tert-butoxycarbonyl)amino)hexan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate (280 mg, 0.384 mmol, 1 eq) in THF (5 mL) was added TBAF (1 M, 2.30 mL, 6 eq) and the reaction mixture was stirred at 20° C. for 3 hours. After the completion of the reaction, the mixture was concentrated in vacuo to afford tert-butyl (2-(((2S,5S)-5-((tert-butoxycarbonyl)amino)hexan-2-yl)oxy)pyridin-4-yl)(1-(tert-butyl)-3-((1S,3R)-3-hydroxycyclopentyl)-1H-pyrazol-5-yl)carbamate (270 mg, 0.384 mmol) as a light-yellow oil, which was used into the next step without further purification. LCMS: 616.5 [M+H]⁺.

Step 11: Preparation of tert-butyl (2-(((2S,5S)-5-((tert-butoxycarbonyl)amino)hexan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(((2S,5S)-5-((tert-butoxycarbonyl)amino)hexan-2-yl)oxy)pyridin-4-yl)(1-(tert-butyl)-3-((1S,3R)-3-hydroxycyclopentyl)-1H-pyrazol-5-yl)carbamate (170 mg, 0.276 mmol, 1 eq) and (4-nitrophenyl) carbonochloridate (166.93 mg, 0.828 mmol, 3 eq) in DCM (20 mL) was added Py (655.10 mg, 8.28 mmol, 30 eq) and DMAP (33.73 mg, 0.276 mmol, 1 eq), and the mixture was stirred at 20° C. for 2 hours under N₂ atmosphere. After the completion of the reaction, the mixture was filtered, rinsed with DCM (10 mL) and concentrated under reduced pressure. The resulting residue was purified by column chromatography to afford tert-butyl (2-(((2S,5S)-5-((tert-butoxycarbonyl)amino)hexan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (170 mg, 79.0% yield) as a light-yellow oil. LCMS: 781.6 [M+H]⁺.

Step 12: Preparation of (1R,3S)-3-(5-((2-(((2S,5S)-5-aminohexan-2-yl)oxy)pyridin-4-yl)amino)-1-(tert-butyl)-1H-pyrazol-3-yl)cyclopentyl (4-nitrophenyl) carbonate

The solution tert-butyl (2-(((2S,5S)-5-((tert-butoxycarbonyl)amino)hexan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (160 mg, 0.205 mmol, 1 eq) and TFA (2.5 g, 21.54 mmol, 1.6 mL) in DCM (3 mL) was stirred at 20° C. for 1 hour. After the completion of the reaction, the mixture was concentrated in vacuo to afford (1R,3S)-3-(5-((2-(((2S,5S)-5-aminohexan-2-yl)oxy)pyridin-4-yl)amino)-1-(tert-butyl)-1H-pyrazol-3-yl)cyclopentyl (4-nitrophenyl) carbonate (140 mg) as a light-yellow oil, which was used in the next step without further purification. LCMS: 581.3 [M+H]⁺.

Step 13: Preparation of (1¹S,1³R,6S,9S,Z)-2¹-(tert-butyl)-6,9-dimethyl-2¹H-5,12-dioxa-3,10-diaza-4(4,2)-pyridina-2(3,5)-pyrazola-1(1,3)-cyclopentanacyclododecaphan-11-one

To a solution of (1R,3S)-3-(5-((2-(((2S,5S)-5-aminohexan-2-yl)oxy)pyridin-4-yl)amino)-1-(tert-butyl)-1H-pyrazol-3-yl)cyclopentyl (4-nitrophenyl) carbonate (130 mg, 0.224 mmol, 1 eq) in THF (200 mL) was added DIEA (2.61 g, 20.19 mmol, 3.5 mL), and the mixture was stirred at 20° C. for 1 hour under N₂ atmosphere. After the completion of the reaction, the mixture was then concentrated in vacuo to afford (1¹S,1³R,6S,9S,Z)-2¹-(tert-butyl)-6,9-dimethyl-2¹H-5,12-dioxa-3,10-diaza-4(4,2)-pyridina-2(3,5)-pyrazola-1(1,3)-cyclopentanacyclododecaphan-11-one (120 mg, 0.272 mmol) as a light-yellow oil, which was used into the next step without further purification. LCMS: 442.3 [M+H]⁺.

Step 11: Preparation of (1¹S,1³R,6S,9S,Z)-6,9-dimethyl-2¹H-5,12-dioxa-3,10-diaza-4(4,2)-pyridina-2(3,5)-pyrazola-1 (1,3)-cyclopentanacyclododecaphan-11-one

A solution of (1¹S,1³R,6S,9S,Z)-2¹-(tert-butyl)-6,9-dimethyl-2¹H-5,12-dioxa-3,10-diaza-4(4,2)-pyridina-2(3,5)-pyrazola-1(1,3)-cyclopentanacyclododecaphan-11-one (110 mg, 0.249 mmol, 1 eq) in formic acid (2 mL) was stirred at 100° C. for 1 hour under N₂ atmosphere. After the completion of the reaction, the mixture was cooled to room temperature, and concentrated under reduced pressure. The resulting residue was purified by prep-HPLC {column: Phenomenex luna C18 150*25 mm*10 um; mobile phase: [water(FA)-ACN]; gradient:5%-35% B over 10 min} to afford (1¹S,1³R,6S,9S,Z)-6,9-dimethyl-2¹H-5,12-dioxa-3,10-diaza-4(4,2)-pyridina-2(3,5)-pyrazola-1(1,3)-cyclopentanacyclododecaphan-11-one (61.7 mg, 0.158 mmol, 98.8% yield) as a white solid. LCMS: 386.2 [M+H]⁺; ¹H NMR (400 MHz, DMSO-d₆) δ 12.75-11.81 (m, 1H), 10.46-9.03 (m, 1H), 7.84-7.79 (m, 1H), 7.05-5.77 (m, 4H), 5.14-4.90 (m, 1H), 4.81-4.50 (m, 1H), 2.46-2.30 (m, 1H), 2.16-1.99 (m, 1H), 1.97-1.68 (m, 1H), 1.67-1.45 (m, 3H), 1.32 (br, 1H), 1.41-1.11 (m, 1H), 1.07-0.89 (m, 2H), 1.06-0.89 (m, 1H) ppm.

Example 31: synthesis of (1¹S,1³R,6S,9R,Z)-6,9-dimethyl-2¹H-5,12-dioxa-3,10-diaza-4(4,2)-pyridina-2(3,5)-pyrazola-1(1,3)-cyclopentanacyclododecaphan-11-one (compound 163)

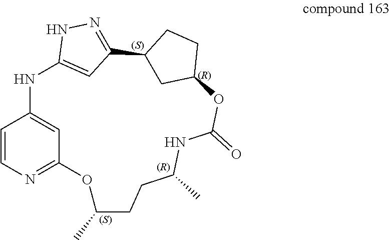

Step 1: Preparation of (S)—N—((S,E)-4-((4-bromopyridin-2-yl)oxy)pentylidene)-2-methylpropane-2-sulfinamide

To a solution of (S)-4-((4-bromopyridin-2-yl)oxy)pentanal (540 mg, 2.09 mmol, 1 eq) and (S)-2-methylpropane-2-sulfinamide (324.57 mg, 2.68 mmol, 1.28 eq) in THF (15 mL) was added CuSO₄ (667.84 mg, 4.18 mmol, 2 eq), and the mixture was stirred at 70° C. for 12 hours under N₂ atmosphere. After the completion of the reaction, the mixture was cooled to room temperature. The mixture was filtered through a pad of the Celite, rinsed with THF (15 mL) and the filtrate was concentrated in vacuo. The resulting residue was purified by prep-TLC (DCM/EtOAc=1/1) to give (S)—N—((S,E)-4-((4-bromopyridin-2-yl)oxy)pentylidene)-2-methylpropane-2-sulfinamide (500 mg, 66.2% yield) as a yellow oil. LCMS: 360.9, 362.9 [M+H]⁺.

Step 2: Preparation of (S)—N-((2R,5S)-5-((4-bromopyridin-2-yl)oxy)hexan-2-yl)-2-methylpropane-2-sulfinamide

To a solution of (S)—N—((S,E)-4-((4-bromopyridin-2-yl)oxy)pentylidene)-2-methylpropane-2-sulfinamide (450 mg, 1.25 mmol, 1 eq) in THF (5 mL) was added MeMgBr (3 M, 1.25 mL, 3 eq) dropwise at 0° C. under N₂ atmosphere. After the completion of the addition, the mixture was warmed to 50° C. and stirred for 12 hours at that temperature under N₂ atmosphere. The mixture was then quenched with saturated aqueous NH₄Cl (20 mL) and extracted with EtOAc (30 mL×2). The combined organic layers were washed with brine (50 mL), dried over anhydrous Na₂SO₄, filtered and the filtrate was concentrated in vacuo. The resulting residue was purified by prep-TLC (DCM/EtOAc=1/1) to give (S)—N-((2R,5S)-5-((4-bromopyridin-2-yl)oxy)hexan-2-yl)-2-methylpropane-2-sulfinamide (300 mg, 63.8% yield) as a yellow oil. LCMS: 377.1, 379.1 [M+H]⁺.

Step 3: Preparation of (2R,5S)-5-((4-bromopyridin-2-yl)oxy)hexan-2-amine

To the solution of (S)—N-((2R,5S)-5-((4-bromopyridin-2-yl)oxy)hexan-2-yl)-2-methylpropane-2-sulfinamide (250 mg, 0.66 mmol, 1 eq) in dioxane (6 mL) was added a solution of HCl in dioxane (4 M, 5.00 mL, 30.19 eq), and the reaction mixture was stirred at 25° C. for 1 hour. After the completion of the reaction, the mixture was concentrated in vacuo to give (2R,5S)-5-((4-bromopyridin-2-yl)oxy)hexan-2-amine (180 mg, 0.65 mmol) as a yellow oil. LCMS: 273.1, 275.1 [M+H]⁺.

Step 4: Preparation of tert-butyl ((2R,5S)-5-((4-bromopyridin-2-yl)oxy)hexan-2-yl)carbamate

To a solution of (2R,5S)-5-((4-bromopyridin-2-yl)oxy)hexan-2-amine (220 mg, 0.81 mmol, 1 eq) and TEA (244.48 mg, 2.42 mmol, 3 eq) in DCM (5 mL) was added Boc₂O (263.65 mg, 1.21 mmol, 1.5 eq), and the mixture was stirred at 25° C. for 12 hours under N₂ atmosphere. After the completion of the reaction, the mixture was concentrated in vacuo. The resulting residue was purified by prep-TLC (PE/EtOAc=3/1) to give tert-butyl ((2R,5S)-5-((4-bromopyridin-2-yl)oxy)hexan-2-yl)carbamate (180 mg, 59.9% yield) as a yellow oil. LCMS: 373.1, 375.1 [M+H]⁺.

Step 5: Preparation of tert-butyl ((2R,5S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)hexan-2-yl)carbamate

A mixture of 1-(tert-butyl)-3-((1S,3R)-3-((tert-butyldimethylsilyl)oxy)cyclopentyl)-1H-pyrazol-5-amine (129.32 mg, 0.38 mmol, 1.1 eq) and tert-butyl ((2R,5S)-5-((4-bromopyridin-2-yl)oxy)hexan-2-yl)carbamate (130 mg, 0.35 mmol, 1 eq), Xantphos (24.18 mg, 0.41 mmol, 0.12 eq), Cs₂CO₃ (340.41 mg, 1.04 mmol, 3 eq) and Pd₂(dba)₃ (20.02 mg, 0.35 mmol, 0.1 eq) in dioxane (5 mL) was stirred at 90° C. under N₂ atmosphere for 3 hours. After the completion of the reaction, the mixture was cooled to room temperature, filtered through a pad of the Celite, rinsed with dioxane (10 mL), and the filtrate was concentrated in vacuo. The resulting residue was purified by prep-TLC (PE/EtOAc=2/1) to give tert-butyl ((2R,5S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)hexan-2-yl)carbamate (219 mg, 99.8% yield) as a yellow oil. LCMS: 630.6 [M+H]⁺.

Step 6: Preparation of tert-butyl (2-(((2S,5R)-5-((tert-butoxycarbonyl)amino)hexan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((2R,5S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)hexan-2-yl)carbamate (219 mg, 0.34 mmol, 1 eq) in DCM (3 mL) was added Boc₂O (113.81 mg, 0.52 mmol, 1.5 eq) and DMAP (21.24 mg, 0.17 mmol, 0.5 eq), and the reaction mixture was stirred at 25° C. for 1 hour. After the completion of the reaction, the mixture was poured into water (20 mL) and extracted with EtOAc (30 mL×2). The combined organic layers were washed with saturated brine (50 mL), dried over anhydrous Na₂SO₄, filtered, and the filtrate was concentrated in vacuo. The resulting residue was purified by prep-TLC (PE/EtOAc=3/1) to give tert-butyl (2-(((2S,5R)-5-((tert-butoxycarbonyl)amino)hexan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate (222 mg, 87.5% yield) as a yellow oil. LCMS: 730.5 [M+H]⁺.

Step 7: Preparation of tert-butyl (2-(((2S,5R)-5-((tert-butoxycarbonyl)amino)hexan-2-yl)oxy)pyridin-4-yl)(1-(tert-butyl)-3-((1S,3R)-3-hydroxycyclopentyl)-1H-pyrazol-5-yl)carbamate

The mixture of tert-butyl (2-(((2S,5R)-5-((tert-butoxycarbonyl)amino)hexan-2-yl)oxy)pyridin-4-yl)(1-(tert-butyl)-3-((1S,3R)-3-((tert-butyldimethylsilyl)oxy)cyclopentyl)-1H-pyrazol-5-yl)carbamate (200 mg, 0.27 mmol, 1 eq) and TBAF (1 M, 1.64 mL, 6 eq) in THF (5 mL) was stirred at 25° C. for 12 hours under N₂ atmosphere. After the completion of the reaction, the reaction mixture was quenched with saturated aqueous NH₄Cl (50 mL) and extracted with EtOAc (20 mL×2). The combined organic layers were washed with brine (50 mL), dried over Na₂SO₄, filtered, and the filtrate was concentrated under reduced pressure to give tert-butyl (2-(((2S,5R)-5-((tert-butoxycarbonyl)amino)hexan-2-yl)oxy)pyridin-4-yl)(1-(tert-butyl)-3-((1S,3R)-3-hydroxycyclopentyl)-1H-pyrazol-5-yl)carbamate (168 mg, 99.5% yield) as a yellow oil. LCMS: 616.4 [M+H]⁺.

Step 8: Preparation of tert-butyl (2-(((2S,5R)-5-((tert-butoxycarbonyl)amino)hexan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

A mixture of tert-butyl (2-(((2S,5R)-5-((tert-butoxycarbonyl)amino)hexan-2-yl)oxy)pyridin-4-yl)(1-(tert-butyl)-3-((1S,3R)-3-hydroxycyclopentyl)-1H-pyrazol-5-yl)carbamate (60 mg, 0.097 mmol, 1 eq), (4-nitrophenyl) carbonochloridate (58.92 mg, 0.29 mmol, 3 eq), DMAP (5.95 mg, 0.048 mmol, 0.5 eq) and Pyridine (46.24 mg, 0.58 mmol, 6 eq) in DCM (3 mL) was stirred at 25° C. for 3 hours under N₂ atmosphere. After the completion of the reaction, the mixture was concentrated in vacuo. The residue was purified by prep-TLC (PE/EtOAc=3/1) to give tert-butyl (2-(((2S,5R)-5-((tert-butoxycarbonyl)amino)hexan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (76 mg, 99.9% yield) as a yellow oil. LCMS: 781.6 [M+H]⁺.

Step 9: Preparation of (1R,3S)-3-(5-((2-(((2S,5R)-5-aminohexan-2-yl)oxy)pyridin-4-yl)amino)-1-(tert-butyl)-1H-pyrazol-3-yl)cyclopentyl (4-nitrophenyl) carbonate

A mixture of tert-butyl (2-(((2S,5R)-5-((tert-butoxycarbonyl)amino)hexan-2-yl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (76 mg, 0.97 mmol, 1 eq) and TFA (33.29 mg, 0.29 mmol, 3 eq) in DCM (2 mL) was stirred at 25° C. for 3 hours. After the completion of the reaction, the mixture was concentrated in vacuo to give (1R,3S)-3-(5-((2-(((2S,5R)-5-aminohexan-2-yl)oxy)pyridin-4-yl)amino)-1-(tert-butyl)-1H-pyrazol-3-yl)cyclopentyl (4-nitrophenyl) carbonate (56 mg, 0.96 mmol, 99.1% yield) as a yellow oil, which was used into the next step without further purification. LCMS: 581.6 [M+H]⁺.

Step 10: Preparation of (1¹S,1³R,6S,9R,Z)-2¹-(tert-butyl)-6,9-dimethyl-2¹H-5,12-dioxa-3,10-diaza-4(4,2)-pyridina-2(3,5)-pyrazola-1(1,3)-cyclopentanacyclododecaphan-11-one

A mixture of (1R,3S)-3-(5-((2-(((2S,5R)-5-aminohexan-2-yl)oxy)pyridin-4-yl)amino)-1-(tert-butyl)-1H-pyrazol-3-yl)cyclopentyl (4-nitrophenyl) carbonate (56 mg, 0.96 mmol, 1 eq) and DIEA (74.78 mg, 0.58 mmol, 6 eq) in THF (50 mL) was stirred at 25° C. for 3 hours under N₂ atmosphere. After the completion of the reaction, the mixture was concentrated in vacuo to give crude (1¹S,1³R,6S,9R,Z)-2¹-(tert-butyl)-6,9-dimethyl-2¹H-5,12-dioxa-3,10-diaza-4(4,2)-pyridina-2(3,5)-pyrazola-1(1,3)-cyclopentanacyclododecaphan-11-one (42 mg) as a yellow oil, which was used in the next step without further purification. LCMS: 442.3 [M+H]⁺.

Step 11: Preparation of (1¹S,1³R,6S,9R,Z)-6,9-dimethyl-2¹H-5,12-dioxa-3,10-diaza-4(4,2)-pyridina-2(3,5)-pyrazola-1 (1,3)-cyclopentanacyclododecaphan-11-one

A mixture of (1¹S,1³R,6S,9R,Z)-2¹-(tert-butyl)-6,9-dimethyl-2¹H-5,12-dioxa-3,10-diaza-4(4,2)-pyridina-2(3,5)-pyrazola-1(1,3)-cyclopentanacyclododecaphan-11-one (42 mg, 0.95 mmol, 1 eq) in HCOOH (2 mL) was stirred at 100° C. for 3 hours under N₂ atmosphere. After the completion of the reaction, the mixture was cooled to room temperature and concentrated in vacuo. The resulting residue was purified by prep-HPLC {column: Waters Xbridge 150*25 mm*Sum; mobile phase: [water (NH₄HCO₃)-ACN]; gradient:15%-45% B over 53 min} and further by SFC {column: DAICEL CHIRALPAK AD (250 mm*30 mm, 10 um); mobile phase: [CO₂-EtOH (0.1% NH₃·H₂O)]; B %:40%, isocratic elution mode} to give (1¹S,1³R,6S,9R,Z)-6,9-dimethyl-2¹H-5,12-dioxa-3,10-diaza-4(4,2)-pyridina-2(3,5)-pyrazola-1(1,3)-cyclopentanacyclododecaphan-11-one (5.03 mg, 0.13 mmol, 13.69% yield, 99.8% purity) as an white solid. LCMS: 386.2 [M+H]⁺; ¹H NMR (400 MHz, DMSO-d₆) δ 9.01-8.77 (m, 1H), 7.81-7.67 (m, 1H), 6.81-6.56 (m, 1H), 6.48-6.26 (m, 2H), 6.07 (s, 1H), 5.20-4.59 (m, 2H), 3.56 (br d, J=6.4 Hz, 1H), 2.40-2.08 (m, 2H), 2.07-1.72 (m, 5H), 1.62-1.50 (m, 3H), 1.30-1.17 (m, 3H), 1.13-0.90 (m, 4H) ppm.

Example 32: synthesis of (1¹S,1³R,9S,Z)-8,8-difluoro-9-methyl-2¹H-6,12-dioxa-3,10-diaza-4(4,2)-pyridina-2(3,5)-pyrazola-1(1,3)-cyclopentanacyclododecaphan-11-one (compound 164) and (1¹S,1³R,9R,Z)-8,8-difluoro-9-methyl-2¹H-6,12-dioxa-3,10-diaza-4(4,2)-pyridina-2(3,5)-pyrazola-1(1,3)-cyclopentanacyclododecaphan-11-one (compound 165)

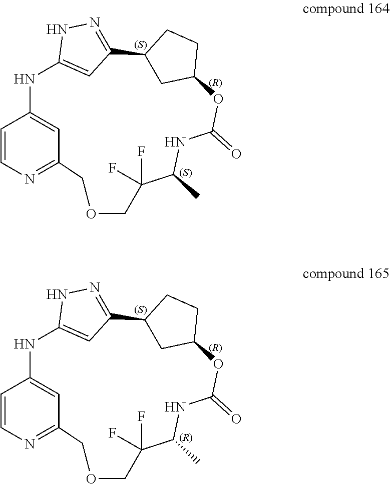

Step 1: Preparation of (S,E)-N-ethylidene-2-methylpropane-2-sulfinamide

A mixture of (S)-2-methylpropane-2-sulfinamide (20 g, 165.02 mmol, 1 eq), acetaldehyde (7 g, 158.90 mmol, 8.92 mL, 0.93 eq), 4-methylbenzenesulfonic acid pyridine (4.15 g, 16.50 mmol, 0.1 eq) and MgSO₄ (20 g, 166.16 mmol, 1.01 eq) in DCM (300 mL) was stirred at 20° C. for 2 hours under N₂ atmosphere. After the completion of the reaction, the mixture was diluted with DCM (300 mL), filtered, rinsed with DCM (50 mL), and the combined filtrate was concentrated. The resulting residue was purified by column chromatography to afford (S,E)-N-ethylidene-2-methylpropane-2-sulfinamide (11 g, 45.3% yield) as a colorless oil. ¹H NMR (400 MHz, CDCl₃) δ 8.04-7.98 (m, 1H), 2.19-2.16 (m, 3H), 1.14-1.10 (m, 9H) ppm.

Step 2: Preparation of ethyl 3-(((S)-tert-butylsulfinyl)amino)-2,2-difluorobutanoate

A mixture of Zn (53.46 g, 817.56 mmol, 12.04 eq) and TMSCl (2.57 g, 23.64 mmol, 3 mL, 0.35 eq) in THF (200 mL) was stirred for 0.5 hour at 20° C. under N₂ atmosphere. Then ethyl 2-bromo-2,2-difluoro-acetate (41 g, 201.99 mmol, 26.03 mL, 2.97 eq) was carefully dropwise and the reaction mixture was maintained below 50° C. After the completion of the addition, the mixture was kept stirring for 0.5 hour at 20° C. under N₂ atmosphere. The mixture was then quenched with water (200 mL) and diluted with ethyl acetate (200 mL). The organic layer was collected, and the aqueous phase was extracted with ethyl acetate (50 mL×2). The combined organic layers were washed with brine (100 mL), dried over anhydrous Na₂SO₄, filtered, and the filtrate was concentrated. The resulting residue was purified by column chromatography to afford ethyl 3-(((S)-tert-butylsulfinyl)amino)-2,2-difluorobutanoate (10 g, 54.3% yield) as a yellow oil. LCMS: 272.1 [M+H]⁺.

Step 3: Preparation of (S)—N-(3,3-difluoro-4-hydroxybutan-2-yl)-2-methylpropane-2-sulfinamide

To a solution of ethyl 3-(((S)-tert-butylsulfinyl)amino)-2,2-difluorobutanoate (4 g, 14.74 mmol, 1 eq) in anhydrous THF (50 mL) was added LiBH₄ (4 M, 11 mL, 3 eq) at 20° C., and the mixture was then warmed to 50° C. and stirred for 4 hours at that temperature under N₂ atmosphere. After the completion of the reaction, the mixture was cooled to room temperature and subsequently quenched with cold saturated aqueous NH₄Cl (100 mL), extracted with ethyl acetate (200 mL×2). The organic layer was washed with brine (100 mL), dried over anhydrous Na₂SO₄, filtered and the filtrate was concentrated. The desired (S)—N-(3,3-difluoro-4-hydroxybutan-2-yl)-2-methylpropane-2-sulfinamide (3 g, 88.7% yield) was obtained as a yellow oil, which was used in the next step without further purification. LCMS: 230.0 [M+H]⁺.

Step 4: Preparation of 3-amino-2,2-difluorobutane-1-ol hydrochloride

A solution of (S)—N-(3,3-difluoro-4-hydroxybutan-2-yl)-2-methylpropane-2-sulfinamide (2.30 g, 10.03 mmol, 1 eq) in HCl/Ethyl acetate (20 mL, 4 M) was stirred for 1 hour at 20° C. After the completion of the reaction, the mixture was concentrated to afford 3-amino-2,2-difluorobutan-1-ol hydrochloride (1.6 g, 9.90 mmol) as a white solid, which was used into the next step without further purification. LCMS: 126.1 [M+H]⁺.

Step 5: Preparation of tert-butyl (3,3-difluoro-4-hydroxybutan-2-yl)carbamate

To a solution of 3-amino-2,2-difluorobutan-1-ol hydrochloride (1.6 g, 9.90 mmol, 1 eq) in MeOH (10 mL) was added a solution of NaOH (792.19 mg, 19.80 mmol, 2 eq) in H₂O (5 mL) and Boc₂O (3.24 g, 14.85 mmol, 3.41 mL, 1.5 eq) at 0° C., and the reaction mixture was then stirred for 2 hours at 20° C. After the completion of the reaction, the mixture was diluted with DCM (300 mL) and washed with water (100 mL). The organic layer was collected and washed with brine (100 mL), dried over anhydrous Na₂SO₄ and filtered. The filtrate was concentrated and the resulting residue was purified by column chromatography to afford tert-butyl (3,3-difluoro-4-hydroxybutan-2-yl)carbamate (1.1 g, 49.32% yield) as a white solid. LCMS: 170.1 [M+H-56]⁺.

Step 6: Preparation of tert-butyl (4-((4-bromopyridin-2-yl)methoxy)-3,3-difluorobutan-2-yl)carbamate

To a solution of 4-bromo-2-(bromomethyl)pyridine (500 mg, 1.99 mmol, 1 eq) and tert-butyl (3,3-difluoro-4-hydroxybutan-2-yl)carbamate (500.00 mg, 2.22 mmol, 1.11 eq) in anhydrous DMF (10 mL) was added t-BuOK (350 mg, 3.12 mmol, 1.57 eq) at 0° C., and then the reaction mixture was warmed to 20° C. and stirred for 3 hours under N₂ atmosphere. After the completion of the reaction, the mixture was quenched with saturated aqueous NH₄Cl (100 mL) and extracted with ethyl acetate (100 mL×2). The organic layer was washed with brine (50 mL), dried over anhydrous Na₂SO₄, filtered and the filtrate was concentrated. The resulting residue was purified by Prep-TLC (Petroleum ether/Ethyl acetate=2:1) to afford tert-butyl (4-((4-bromopyridin-2-yl)methoxy)-3,3-difluorobutan-2-yl)carbamate (230 mg, 0.58193 mmol, 29.2% yield) as a yellow solid. LCMS: 295.1, 297.1 [M+H]⁺.

Step 7: Preparation of tert-butyl (4-((4-((1-(tert-butyl)-3-((1S,3R)-3-((tert-butyldimethylsilyl)oxy)cyclopentyl)-1H-pyrazol-5-yl)amino)pyridin-2-yl)methoxy)-3,3-difluorobutan-2-yl)carbamate

A mixture of tert-butyl (4-((4-bromopyridin-2-yl)methoxy)-3,3-difluorobutan-2-yl)carbamate (200 mg, 0.50 mmol, 1 eq), 1-(tert-butyl)-3-((1S,3R)-3-((tert-butyldimethylsilyl)oxy)cyclopentyl)-1H-pyrazol-5-amine (170.82 mg, 0.51 mmol, 1 eq), Cs₂CO₃ (329.74 mg, 1.01 mmol, 2 eq), Xantphos (58.56 mg, 0.10 mmol, 0.2 eq) and Pd₂(dba)₃ (92.67 mg, 0.10 mmol, 0.2 eq) in dioxane (4 mL) was degassed and backfilled with N₂ for 3 times, and stirred at 90° C. for 4 hours under N₂ atmosphere. After the completion of the reaction, the mixture was cooled to room temperature, diluted with H₂O (50 mL) and extracted with Ethyl acetate (50 mL×3). The combined organic layers were washed with brine (50 mL), dried over Na₂SO₄, filtered and the filtrate was concentrated under reduced pressure. The resulting residue was purified by column chromatography to afford tert-butyl (4-((4-((1-(tert-butyl)-3-((1S,3R)-3-((tert-butyldimethylsilyl)oxy)cyclopentyl)-1H-pyrazol-5-yl)amino)pyridin-2-yl)methoxy)-3,3-difluorobutan-2-yl)carbamate (256 mg, 77.6% yield) as a yellow solid. LCMS: 652.5 [M+H]⁺.

Step 8: Preparation of tert-butyl (2-((3-((tert-butoxycarbonyl)amino)-2,2-difluorobutoxy)methyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl (4-((4-((1-(tert-butyl)-3-((1S,3R)-3-((tert-butyldimethylsilyl)oxy)cyclopentyl)-1H-pyrazol-5-yl)amino)pyridin-2-yl)methoxy)-3,3-difluorobutan-2-yl)carbamate (236.0 mg, 0.36 mmol, 1 eq) and DMAP (44.23 mg, 0.36 mmol, 1 eq) in DCM (5 mL) was added Boc₂O (158.02 mg, 0.72 mmol, 0.17 ml, 2 eq), and the mixture was stirred at 25° C. for 4 hours under N₂ atmosphere. After the completion of the reaction, the reaction mixture was concentrated under reduced pressure. The resulting residue was purified by column chromatography to afford tert-butyl (2-((3-((tert-butoxycarbonyl)amino)-2,2-difluorobutoxy)methyl)pyridin-4-yl)(1-(tert-butyl)-3-((1S,3R)-3-((tert-butyldimethylsilyl)oxy)cyclopentyl)-1H-pyrazol-5-yl)carbamate (240 mg, 88.2% yield) as a yellow oil. LCMS: 752.7 [M+H]⁺.

Step 9: Preparation of tert-butyl (2-((3-((tert-butoxycarbonyl)amino)-2,2-difluorobutoxy)methyl)pyridin-4-yl)(1-(tert-butyl)-3-((1S,3R)-3-hydroxycyclopentyl)-1H-pyrazol-5-yl)carbamate

The solution of tert-butyl (2-((3-((tert-butoxycarbonyl)amino)-2,2-difluorobutoxy)methyl)pyridin-4-yl)(1-(tert-butyl)-3-((1S,3R)-3-((tert-butyldimethylsilyl)oxy)cyclopentyl)-1H-pyrazol-5-yl)carbamate (230 mg, 0.30 mmol, 1 eq) and TBAF (1 M, 1.84 mL, 6 eq) in THF (2 mL) was stirred at 25° C. for 4 hours under N₂ atmosphere. After the completion of the reaction, the mixture was diluted with H₂O (15 mL) and extracted with ethyl acetate (30 mL×3). The combined organic layers were washed with brine (30 mL×2), dried over Na₂SO₄, filtered and the filtrate was concentrated under reduced pressure. The desired tert-butyl (2-((3-((tert-butoxycarbonyl)amino)-2,2-difluorobutoxy)methyl)pyridin-4-yl)(1-(tert-butyl)-3-((1S,3R)-3-hydroxycyclopentyl)-1H-pyrazol-5-yl)carbamate (160 mg, 0.25 mmol) as a yellow oil, which was used in the next step without further purification. LCMS: 638.5 [M+H]⁺.

Step 10: Preparation of tert-butyl (2-((3-((tert-butoxycarbonyl)amino)-2,2-difluorobutoxy)methyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-((3-((tert-butoxycarbonyl)amino)-2,2-difluorobutoxy)methyl)pyridin-4-yl)(1-(tert-butyl)-3-((1S,3R)-3-hydroxycyclopentyl)-1H-pyrazol-5-yl)carbamate (150 mg, 0.23 mmol, 1 eq) and (4-nitrophenyl) carbonochloridate (94.82 mg, 0.47 mmol, 2 eq) in DCM (4 mL) was added Pyridine (111.63 mg, 1.41 mmol, 0.11 mL, 6 eq) and DMAP (28.73 mg, 0.23 mmol, 1 eq), and the mixture was stirred at 25° C. for 2 hours under N₂ atmosphere. After the completion of the reaction, the reaction mixture was diluted with H₂O (15 mL) and extracted with DCM (10 mL×3). The combined organic layers were washed with brine (10 mL×2), dried over Na₂SO₄, filtered and the filtrate was concentrated under reduced pressure.

The resulting residue was purified by column chromatography to afford tert-butyl (2-((3-((tert-butoxycarbonyl)amino)-2,2-difluorobutoxy)methyl)pyridin-4-yl)(1-(tert-butyl)-3-((1S,3R)-3-(((4-nitrophenoxy)carbonyl)oxy)cyclopentyl)-1H-pyrazol-5-yl)carbamate (120 mg, 63.6% yield) as a yellow oil. LCMS: 803.5 [M+H]⁺.

Step 11: Preparation of (1R,3S)-3-(5-((2-((3-amino-2,2-difluorobutoxy)methyl)pyridin-4-yl)amino)-1-(tert-butyl)-1H-pyrazol-3-yl)cyclopentyl (4-nitrophenyl) carbonate

The solution of tert-butyl (2-((3-((tert-butoxycarbonyl)amino)-2,2-difluorobutoxy)methyl)pyridin-4-yl)(1-(tert-butyl)-3-((1S,3R)-3-(((4-nitrophenoxy)carbonyl)oxy)cyclopentyl)-1H-pyrazol-5-yl)carbamate (120.00 mg, 0.15 mmol, 1 eq) and TFA (1.54 g, 13.46 mmol, 90 eq) in DCM (2 mL) was stirred at 25° C. for 12 hours. After the completion of the reaction, the reaction mixture was concentrated under reduced pressure to afford the crude (1R,3S)-3-(5-((2-((3-amino-2,2-difluorobutoxy)methyl)pyridin-4-yl)amino)-1-(tert-butyl)-1H-pyrazol-3-yl)cyclopentyl (4-nitrophenyl) carbonate (80 mg, 0.13 mmol, 88.8% yield) as a yellow oil, which was used in the next step without further purification. LCMS: 603.4 [M+H]⁺.

Step 12: Preparation of (1¹S,1³R,Z)-2¹-(tert-butyl)-8,8-difluoro-9-methyl-2¹H-6,12-dioxa-3,10-diaza-4(4,2)-pyridina-2(3,5)-pyrazola-1(1,3)-cyclopentanacyclododecaphan-11-one

To a solution of (1R,3S)-3-(5-((2-((3-amino-2,2-difluorobutoxy)methyl)pyridin-4-yl)amino)-1-(tert-butyl)-1H-pyrazol-3-yl)cyclopentyl (4-nitrophenyl) carbonate (80.00 mg, 0.13 mmol, 1 eq) in THF (100 mL) was added DIPEA (102.9 mg, 0.80 mmol, 0.14 mL, 6 eq), and the mixture was stirred at 80° C. for 16 hours under N₂ atmosphere. After the completion of the reaction, the mixture was cooled to room temperature. The reaction mixture was concentrated under reduced pressure to afford the crude (1¹S,1³R,Z)-2¹-(tert-butyl)-8,8-difluoro-9-methyl-2¹H-6,12-dioxa-3,10-diaza-4(4,2)-pyridina-2(3,5)-pyrazola-1(1,3)-cyclopentanacyclododecaphan-11-one (40 mg, 0.086 mmol) as a brown oil, which was used into the next step without further purification. LCMS: 464.3 [M+H]⁺.

Step 13: Preparation of (1¹S,1³R,9S,Z)-8,8-difluoro-9-methyl-2¹H-6,12-dioxa-3,10-diaza-4(4,2)-pyridina-2(3,5)-pyrazola-1(1,3)-cyclopentanacyclododecaphan-11-one and (1¹S,1³R,9R,Z)-8,8-difluoro-9-methyl-2¹H-6,12-dioxa-3,10-diaza-4(4,2)-pyridina-2(3,5)-pyrazola-1(1,3)-cyclopentanacyclododecaphan-11-one

A solution of (1¹S,1³R,Z)-2¹-(tert-butyl)-8,8-difluoro-9-methyl-2¹H-6,12-dioxa-3,10-diaza-4(4,2)-pyridina-2(3,5)-pyrazola-1(1,3)-cyclopentanacyclododecaphan-11-one (30.0 mg, 0.06472 mmol, 1 eq) in formic acid (1 mL) was stirred at 100° C. for 4 hours under N₂ atmosphere. After the completion of the reaction, the mixture was cooled to room temperature and the volatiles were removed under reduced pressure. The resulting residue was purified by prep-HPLC (column: Phenomenex luna C18 150*25 mm*10 um; mobile phase: [water(FA)-ACN]; gradient:5%-35% B over 10 min) and further separated by SFC (column: DAICEL CHIRALPAK AS(250 mm*30 mm, 10 um); mobile phase: [CO₂-EtOH(0.1% NH₃H₂O)]; B %:35%, isocratic elution mode) to afford (1¹S,1³R,9S,Z)-8,8-difluoro-9-methyl-2¹H-6,12-dioxa-3,10-diaza-4(4,2)-pyridina-2(3,5)-pyrazola-1(1,3)-cyclopentanacyclododecaphan-11-one (15.63 mg, 58.8% yield) as a white solid and (1¹S,1³R,9R,Z)-8,8-difluoro-9-methyl-2¹H-6,12-dioxa-3,10-diaza-4(4,2)-pyridina-2(3,5)-pyrazola-1(1,3)-cyclopentanacyclododecaphan-11-one (3.02 mg, 10.9% yield) was obtained as a white solid.

(1¹S,1³R,9S,Z)-8,8-difluoro-9-methyl-2¹H-6,12-dioxa-3,10-diaza-4(4,2)-pyridina-2(3,5)-pyrazola-1(1,3)-cyclopentanacyclododecaphan-11-one LCMS: 408.2 [M+H]⁺; ¹H NMR (400 MHz, DMSO-d₆) δ 9.29-8.79 (m, 1H), 8.04 (s, 1H), 7.27-7.02 (m, 2H), 6.67 (s, 1H), 5.94 (s, 1H), 5.11-5.05 (d, J=8.0 Hz, 1H), 4.54-4.50 (m, 2H), 4.09-3.60 (m, 4H), 3.20-3.19 (m, 1H), 2.08-2.04 (m, 2H), 1.84-1.73 (m, 4H), 1.15-1.05 (m, 3H) ppm.

(1¹S,1³R,9R,Z)-8,8-difluoro-9-methyl-2¹H-6,12-dioxa-3,10-diaza-4(4,2)-pyridina-2(3,5)-pyrazola-1(1,3)-cyclopentanacyclododecaphan-11-one LCMS: 408.2 [M+H]⁺; ¹H NMR (400 MHz, DMSO-d₆) 5=9.20-9.04 (m, 1H), 8.04 (d, J=5.6 Hz, 1H), 7.10-7.03 (m, 2H), 6.73-6.67 (m, 1H), 5.99 (s, 1H), 5.20-5.12 (m, 1H), 4.57-4.51 (m, 2H), 4.20-4.19 (m, 1H), 3.88-3.82 (m, 2H), 3.21-3.18 (m, 1H), 2.04-1.96 (m, 3H), 1.86-1.69 (m, 4H), 1.14-1.12 (d, J=6.8 Hz, 3H) ppm.

Example 33: synthesis of (1¹S,1³R,9S,Z)-5,5-difluoro-9-methyl-2¹H-12-oxa-3,10-diaza-4(4,2)-pyridina-2(3,5)-pyrazola-1(1,3)-cyclopentanacyclododecaphan-11-one (compound 166)

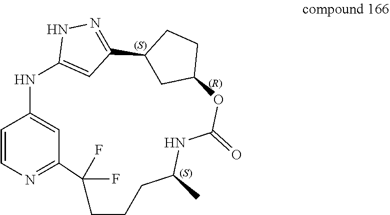

Step 1: Preparation of 2-((bromotriphenyl-λ⁵-phosphaneyl)difluoromethyl)-4-chloropyridine

To a solution of 4-chloropyridine 1-oxide (4.66 g, 36.00 mmol, 1 eq) in DCM (36 mL) was added methyl trifluoromethanesulfonate (5.91 g, 36.00 mmol, 1 eq) dropwise at 25° C. under N₂ atmosphere, and the mixture was stirred for 1 hour at that temperature under N₂ atmosphere. The mixture was then cooled to −20° C. and evaporated under reduced pressure. The resulting residue was dissolved in CH₃CN (72 mL) and the solution was cooled to −30° C. To the solution was successively added (bromodifluoromethyl)trimethylsilane (32.91 g, 162.06 mmol, 4.50 eq) and triphenylphosphane (28.34 g, 108.04 mmol, 3.00 eq), and the mixture was further cooled to −40° C. and HMPA (25.81 g, 144.05 mmol, 25.21 mL, 4.00 eq) was then added dropwise at −40° C. under N₂ atmosphere. After the completion of the addition, the reaction mixture was slowly warmed to 25° C. and stirred for 16 hours under N₂ atmosphere. After the completion of the reaction, the reaction was cooled to −40° C. again and stood still for 4 hours under N₂ atmosphere. The clear liquid was decanted, and the remaining residue was washed with a cold mixed solvent of methyl tert-butyl ether and MeCN (ratio 4:1) (5 mL×3). The solid was then recrystallized from small amount of acetonitrile, washed with a cold mixed solvent of methyl tert-butyl ether and MeCN (ratio 4:1) (5 mL×3) and dried to give 2-((bromotriphenyl-λ⁵-phosphaneyl)difluoromethyl)-4-chloropyridine (10 g, 55.0% yield) as a grey solid, which was used into the next step without further purification. 1H NMR (400 MHz, CHLOROFORM-d) δ 8.56-8.55 (d, J=5.2 Hz, 1H), 7.90-7.88 (m, 1H), 7.76-7.58 (m, 15H), 7.27 (s, 1H) ppm.

Step 2: Preparation of tert-butyl 4-(4-chloropyridin-2-yl)-4,4-difluorobutanoate

To a solution of 2-((bromotriphenyl-λ⁵-phosphaneyl)difluoromethyl)-4-chloropyridine (2 g, 3.96 mmol, 1 eq) and tert-butyl acrylate (0.26 g, 2.03 mmol, 0.51 eq) in DCM (10 mL) was added DIEA (371.00 mg, 2.87 mmol, 0.5 mL, 0.72 eq) and diethyl 2,6-dimethyl-1,4-dihydropyridine-3,5-dicarboxylate (1.00 g, 3.96 mmol, 1 eq) under N₂ atmosphere. The reaction mixture was irradiated using blue LED and stirred at 20° C. for 2 hours. 15 batches were conducted in parallel. After the completion of the reaction, the combined reaction solution was concentrated in vacuo to give tert-butyl 4-(4-chloropyridin-2-yl)-4,4-difluorobutanoate (10 g, 34.28 mmol, 57.7% yield) as a yellow oil, which was used into the next step without further purification. LCMS: 236.1 [M-C₄H₉]⁺.

Step 3: Preparation of 4-(4-chloropyridin-2-yl)-4,4-difluorobutan-1-ol

To a solution of tert-butyl 4-(4-chloropyridin-2-yl)-4,4-difluorobutanoate (5 g, 17.14 mmol, 1 eq) in THF (50 mL) was added LiBH₄ (2 M, 18 mL, 2.10 eq), and the mixture was stirred at 20° C. for 16 hours under N₂ atmosphere. After the reaction was completed, the reaction mixture was diluted with saturated aqueous NH₄Cl (100 mL) and extracted with EtOAc (100 mL). The organic phase was washed with brine (100 mL), dried over Na₂SO₄, filtered, and the filtrate was concentrated in vacuo. The resulting residue was purified by column chromatography to give 4-(4-chloropyridin-2-yl)-4,4-difluorobutan-1-ol (2.7 g, 12.18 mmol, 71.1% yield) as a yellow oil. LCMS: 222.0 [M+H]⁺.

Step 4: Preparation of 4-(4-chloropyridin-2-yl)-4,4-difluorobutyl methanesulfonate

To a solution of 4-(4-chloropyridin-2-yl)-4,4-difluorobutan-1-ol (2.7 g, 12.18 mmol, 1 eq) in THF (100 mL) was added methylsulfonyl methanesulfonate (4.34 g, 24.93 mmol, 2.05 eq) and DIEA (5.21 g, 40.30 mmol, 7.02 mL, 3.31 eq), and the mixture was stirred at 25° C. for 16 hours under N₂ atmosphere. After the completion of the reaction, the reaction mixture was diluted with water (100 mL) and extracted with EtOAc (200 mL). The organic phase was washed with brine (100 mL), dried over Na₂SO₄, filtered, and the filtrate was concentrated in vacuum to give 4-(4-chloropyridin-2-yl)-4,4-difluorobutyl methanesulfonate (2.9 g, 9.68 mmol, 79.4% yield) as a yellow oil, which was used into the next step without further purification. LCMS: 300.0 [M+H]⁺.

Step 5: Preparation of 5-(4-chloropyridin-2-yl)-5,5-difluoropentanenitrile

To a solution of 4-(4-chloropyridin-2-yl)-4,4-difluorobutyl methanesulfonate (2.9 g, 9.68 mmol, 1 eq) in DMF (30 mL) was added NaCN (1.69 g, 34.48 mmol, 3.56 eq), and the mixture was stirred at 20° C. for 16 hours under N₂ atmosphere. After the reaction was completed, the mixture was diluted with water (100 mL) and extracted with EtOAc (100 mL). The organic phase was washed with brine (50 mL), dried over Na₂SO₄, filtered, and the filtrate was concentrated in vacuo. The resulting residue was purified by column chromatography to give 5-(4-chloropyridin-2-yl)-5,5-difluoropentanenitrile (1.1 g, 4.77 mmol, 49.3% yield) as a yellow oil. LCMS: 231.0 [M+H]⁺.

Step 6: Preparation of 5-(4-chloropyridin-2-yl)-5,5-difluoropentanal

To a solution of 5-(4-chloropyridin-2-yl)-5,5-difluoropentanenitrile (0.5 g, 2.17 mmol, 1 eq) in DCM (10 mL) was added DIBAL-H (1 M, 2.50 mL, 1.15 eq) at −78° C., and the reaction mixture was stirred for 1 hour at that temperature under N₂ atmosphere. After the reaction was completed, the reaction mixture was diluted with water (10 mL) and extracted with DCM (50 mL). The organic phase was dried over Na₂SO₄, filtered, and the filtrate was concentrated in vacuum. The resulting residue was purified by column chromatography to give 5-(4-chloropyridin-2-yl)-5,5-difluoropentanal (0.4 g, 79.0% yield) as a yellow oil. LCMS: 234.0 [M+H]⁺.

Step 7: Preparation of (R,E)-N-(5-(4-chloropyridin-2-yl)-5,5-difluoropentylidene)-2-methylpropane-2-sulfinamide

To a solution of 5-(4-chloropyridin-2-yl)-5,5-difluoropentanal (400 mg, 1.71 mmol, 1 eq) in THF (10 mL) was added (R)-2-methylpropane-2-sulfinamide (420 mg, 3.47 mmol, 2.02 eq) and CuSO₄ (550 mg, 3.45 mmol, 0.53 mL, 2.01 eq) and 4 Å molecular sieve (0.5 g), and the mixture was stirred at 60° C. for 3 hours under N₂ atmosphere. After the reaction was completed, the reaction mixture was diluted with water (10 mL) and extracted with DCM (50 mL). The organic phase was dried over Na₂SO₄, filtered, and the filtrate was concentrated in vacuum. The resulting residue was purified by column chromatography to give (R,E)-N-(5-(4-chloropyridin-2-yl)-5,5-difluoropentylidene)-2-methylpropane-2-sulfinamide (0.2 g, 0.59 mmol, 34.7% yield) as a yellow oil. LCMS: 337.2 [M+H]⁺.

Step 8: (R)—N—((S)-6-(4-chloropyridin-2-yl)-6,6-difluorohexan-2-yl)-2-methylpropane-2-sulfinamide

To a solution of (R,E)-N-(5-(4-chloropyridin-2-yl)-5,5-difluoropentylidene)-2-methylpropane-2-sulfinamide (200 mg, 0.59 mmol, 1 eq) in THF (1 mL) was added MeMgBr (3 M, 0.20 mL, 1.01 eq) at 0° C. under N₂ atmosphere. After the completion of the addition, the mixture was warmed to 20° C. and stirred for 1 hour. The mixture was then diluted with saturated aqueous NH₄Cl (50 mL) and extracted with EtOAc (100 mL).

The organic phase was washed with brine (50 mL), dried over Na₂SO₄, filtered, and the filtrate was concentrated in vacuum. The desired (R)—N—((S)-6-(4-chloropyridin-2-yl)-6,6-difluorohexane-2-yl)-2-methylpropane-2-sulfinamide (200 mg, 95.5% yield) was obtained as a yellow oil, which was used in the next step without further purification. LCMS: 353.2 [M+H]⁺.

Step 9: Preparation of (S)-6-(4-chloropyridin-2-yl)-6,6-difluorohexan-2-amine

To a solution of (R)—N—((S)-6-(4-chloropyridin-2-yl)-6,6-difluorohexan-2-yl)-2-methylpropane-2-sulfinamide (200.00 mg, 0.56 mmol, 1 eq) in dioxane (4 mL) was added HCl/dioxane (4 M, 4.00 mL, 28.23 eq), and the mixture was heated to 90° C. for 0.5 hour. After the completion of the reaction, the mixture was cooled to room temperature and concentrated in vacuo. The desired (S)-6-(4-chloropyridin-2-yl)-6,6-difluorohexan-2-amine (150 mg) was obtained as a brown oil, which was used into the next step without further purification. LCMS: 249.1 [M+H]⁺.

Step 10: Preparation of tert-butyl (S)-(6-(4-chloropyridin-2-yl)-6,6-difluorohexan-2-yl)carbamate

To a solution of (S)-6-(4-chloropyridin-2-yl)-6,6-difluorohexan-2-amine (150.00 mg, 0.6 mmol, 1 eq) and TEA (727.00 mg, 7.18 mmol, 1 mL, 11.91 eq) in DCM (4 mL) was added (Boc)₂O (197.45 mg, 0.9 mmol, 1.5 eq), and the mixture was stirred at 25° C. for 1 hour under N₂ atmosphere. After the completion of the reaction, the mixture was diluted with water (10 mL) and extracted with DCM (50 mL). The organic phase was dried over Na₂SO₄, filtered, and the filtrate was concentrated in vacuum. The resulting residue was purified by column chromatography to give tert-butyl (S)-(6-(4-chloropyridin-2-yl)-6,6-difluorohexan-2-yl)carbamate (110 mg, 52.3% yield) as a colorless oil. LCMS: 349.2 [M+H]⁺.

Step 11: Preparation of tert-butyl ((S)-6-(4-((1-(tert-butyl)-3-((1S,3R)-3-((tert-butyldimethylsilyl)oxy)cyclopentyl)-1H-pyrazol-5-yl)amino)pyridin-2-yl)-6,6-difluorohexan-2-yl)carbamate

A mixture of tert-butyl (S)-(6-(4-chloropyridin-2-yl)-6,6-difluorohexan-2-yl)carbamate (110 mg, 0.31 mmol, 1 eq), 1-(tert-butyl)-3-((1S,3R)-3-((tert-butyldimethylsilyl)oxy)cyclopentyl)-1H-pyrazol-5-amine (117.10 mg, 0.34 mmol, 1.1 eq), Pd₂(dba)₃ (28.88 mg, 0.31 mmol, 0.1 eq), Xantphos (36.49 mg, 0.63 mmol, 0.2 eq) and Cs₂CO₃ (308.24 mg, 0.95 mmol, 3 eq) in dioxane (5 mL) was degassed and backfilled with N₂ for 3 times, and then heated and stirred at 90° C. for 1 hour under N₂ atmosphere. After the completion of the reaction, the mixture was cooled to room temperature. The reaction mixture was diluted with water (10 mL) and extracted with DCM (50 mL). The organic phase was dried over Na₂SO₄, filtered, and the filtrate was concentrated in vacuum. The resulting residue was purified by column chromatography to give tert-butyl ((S)-6-(4-((1-(tert-butyl)-3-((1S,3R)-3-((tert-butyldimethylsilyl)oxy)cyclopentyl)-1H-pyrazol-5-yl)amino)pyridin-2-yl)-6,6-difluorohexan-2-yl)carbamate (230 mg, 72.9% yield) as a light-yellow oil.

Step 12: Preparation of tert-butyl (2-((S)-5-((tert-butoxycarbonyl)amino)-1,1-difluorohexyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-6-(4-((1-(tert-butyl)-3-((1S,3R)-3-((tert-butyldimethylsilyl)oxy)cyclopentyl)-1H-pyrazol-5-yl)amino)pyridin-2-yl)-6,6-difluorohexan-2-yl)carbamate (190 mg, 0.29 mmol, 1 eq) in DCM (5 mL) was added Boc₂O (127.60 mg, 0.58 mmol, 2 eq) and DMAP (17.86 mg, 0.15 mmol, 0.5 eq), and the mixture was stirred at 25° C. for 12 hours under N₂ atmosphere. After the reaction was completed, the reaction mixture was diluted with water (10 mL) and extracted with DCM (50 mL). The organic phase was dried over Na₂SO₄, filtered, and the filtrate was concentrated in vacuum. The desired tert-butyl (2-((S)-5-((tert-butoxycarbonyl)amino)-1,1-difluorohexyl)pyridin-4-yl)(1-(tert-butyl)-3-((1S,3R)-3-((tert-butyldimethylsilyl)oxy)cyclopentyl)-1H-pyrazol-5-yl)carbamate (219 mg, 0.29 mmol, 99.8% yield) was obtained as a yellow oil, which was used into the next step without further purification. LCMS: 750.7 [M+H]⁺.

Step 13: Preparation of tert-butyl (2-((S)-6-((tert-butoxycarbonyl)amino)-1,1-difluoroheptyl)pyridin-4-yl)(1-(tert-butyl)-3-((1S,3R)-3-hydroxycyclopentyl)-1H-pyrazol-5-yl)carbamate

The mixture of tert-butyl (2-((S)-5-((tert-butoxycarbonyl)amino)-1,1-difluorohexyl)pyridin-4-yl)(1-(tert-butyl)-3-((1S,3R)-3-((tert-butyldimethylsilyl)oxy)cyclopentyl)-1H-pyrazol-5-yl)carbamate (219 mg, 0.29 mmol, 1 eq) and TBAF (1 M, 1.75 mL, 6 eq) in THF (3 mL) was stirred at 25° C. for 1 hour under N₂ atmosphere. The mixture was then quenched with NH₄Cl (50 mL) and extracted with EtOAc (30 mL×3). The combined organic layers were washed with brine (50 mL×3), dried over Na₂SO₄, filtered, and the filtrate was concentrated under reduced pressure. The resulting tert-butyl (2-((S)-6-((tert-butoxycarbonyl)amino)-1,1-difluoroheptyl)pyridin-4-yl)(1-(tert-butyl)-3-((1S,3R)-3-hydroxycyclopentyl)-1H-pyrazol-5-yl)carbamate (228 mg) was obtained as a yellow oil, which was used in the next step without further purification.

Step 14: Preparation of tert-butyl (2-((S)-5-((tert-butoxycarbonyl)amino)-1,1-difluorohexyl)pyridin-4-yl)(1-(tert-butyl)-3-((1S,3R)-3-(((4-nitrophenoxy)carbonyl)oxy)cyclopentyl)-1H-pyrazol-5-yl)carbamate

A mixture of tert-butyl (2-((S)-6-((tert-butoxycarbonyl)amino)-1,1-difluoroheptyl)pyridin-4-yl)(1-(tert-butyl)-3-((1S,3R)-3-hydroxycyclopentyl)-1H-pyrazol-5-yl)carbamate (228 mg, 0.36 mmol, 1 eq), (4-nitrophenyl) carbonochloridate (216.85 mg, 1.08 mmol, 3 eq), Pyridine (170.20 mg, 2.15 mmol, 6 eq) and DMAP (21.91 mg, 0.18 mmol, 0.5 eq) in DCM (3 mL) was stirred at 25° C. for 3 hours under N₂ atmosphere. After the completion of the reaction, the mixture was quenched with saturated aqueous NH₄Cl (50 mL) and extracted with EtOAc (30 mL×3). The combined organic layers were washed with brine (50 mL), dried over Na₂SO₄, filtered, and the filtrate was concentrated under reduced pressure. The resulting residue was purified by column chromatography to give tert-butyl (2-((S)-5-((tert-butoxycarbonyl)amino)-1,1-difluorohexyl)pyridin-4-yl)(1-(tert-butyl)-3-((1S,3R)-3-(((4-nitrophenoxy)carbonyl)oxy)cyclopentyl)-1H-pyrazol-5-yl)carbamate (130 mg, 45.3% yield) as a yellow oil. LCMS: 801.4 [M+H]⁺.

Step 15: Preparation of (1R,3S)-3-(5-((2-((S)-5-amino-1,1-difluorohexyl)pyridin-4-yl)amino)-1-(tert-butyl)-1H-pyrazol-3-yl)cyclopentyl (4-nitrophenyl) carbonate

A mixture of tert-butyl (2-((S)-5-((tert-butoxycarbonyl)amino)-1,1-difluorohexyl)pyridin-4-yl)(1-(tert-butyl)-3-((1S,3R)-3-(((4-nitrophenoxy)carbonyl)oxy)cyclopentyl)-1H-pyrazol-5-yl)carbamate (130 mg, 0.16 mmol, 1 eq) and TFA (55.52 mg, 0.49 mmol, 3 eq) in DCM (2 mL) was stirred at 25° C. for 3 hours under N₂ atmosphere. After the completion of the reaction, the mixture was concentrated under reduced pressure to give (1R,3S)-3-(5-((2-((S)-5-amino-1,1-difluorohexyl)pyridin-4-yl)amino)-1-(tert-butyl)-1H-pyrazol-3-yl)cyclopentyl (4-nitrophenyl) carbonate (97 mg, 0.16 mmol) as a yellow oil, which was used in the next step without further purification. LCMS: 462.3 [M+H]⁺.

Step 16: Preparation of (1¹S,1³R,9S,Z)-2¹-(tert-butyl)-5,5-difluoro-9-methyl-2¹H-12-oxa-3,10-diaza-4(4,2)-pyridina-2(3,5)-pyrazola-1(1,3)-cyclopentanacyclododecaphan-11-one

A mixture of (1R,3S)-3-(5-((2-((S)-5-amino-1,1-difluorohexyl)pyridin-4-yl)amino)-1-(tert-butyl)-1H-pyrazol-3-yl)cyclopentyl (4-nitrophenyl) carbonate (97 mg, 0.16 mmol, 1 eq) and DIEA (62.61 mg, 0.48 mmol, 3 eq) in THF (120 mL) was stirred at 25° C. for 3 hours under N₂ atmosphere. The mixture was then concentrated under reduced pressure to give (1¹S,1³R,9S,Z)-2¹-(tert-butyl)-5,5-difluoro-9-methyl-2¹H-12-oxa-3,10-diaza-4(4,2)-pyridina-2(3,5)-pyrazola-1(1,3)-cyclopentanacyclododecaphan-11-one (74 mg, 0.16 mmol) as a yellow oil, which was used in the next step without further purification. LCMS: 462.3 [M+H]⁺.

Step 17: Preparation of (1¹S,1³R,9S,Z)-5,5-difluoro-9-methyl-2¹H-12-oxa-3,10-diaza-4(4,2)-pyridina-2(3,5)-pyrazola-1(1,3)-cyclopentanacyclododecaphan-11-one

A mixture of (1¹S,1³R,9S,Z)-2¹-(tert-butyl)-5,5-difluoro-9-methyl-2¹H-12-oxa-3,10-diaza-4(4,2)-pyridina-2(3,5)-pyrazola-1(1,3)-cyclopentanacyclododecaphan-11-one (74 mg, 0.16 mmol, 1 eq) in HCOOH (3 mL) was stirred at 100° C. for 3 hours under N₂ atmosphere. After the completion of the reaction, the mixture was cooled to room temperature and concentrated under reduced pressure. The resulting residue was purified by prep-HPLC {column: Phenomenex luna C18 150*25 mm*10 um; mobile phase: [water (FA)-ACN]; gradient:10%-40% B over 10 min} to give (1¹S,1³R,9S,Z)-5,5-difluoro-9-methyl-2¹H-12-oxa-3,10-diaza-4(4,2)-pyridina-2(3,5)-pyrazola-1(1,3)-cyclopentanacyclododecaphan-11-one (27.15 mg, 0.65 mmol, 40.81% yield, 97.7% purity) as a white solid. LCMS: 406.2 [M+H]⁺; ¹H NMR (400 MHz, DMSO-d₆) δ 8.83 (s, 1H), 8.20 (d, J=5.6 Hz, 1H), 7.04-6.87 (m, 2H), 6.81 (dd, J=1.9, 5.5 Hz, 1H), 5.88 (s, 1H), 5.01-4.93 (m, 1H), 3.61-3.53 (m, 1H), 3.21 (br d, J=3.1 Hz, 1H), 2.43 (dt, J=5.6, 10.4 Hz, 1H), 2.26-2.08 (m, 2H), 2.06-2.02 (m, 1H), 1.83-1.70 (m, 4H), 1.50-1.40 (m, 1H), 1.32-1.17 (m, 3H), 0.96 (br d, J=6.6 Hz, 3H) ppm.

Example 34: synthesis of (1¹S,1³R,2⁴Z,9S)-5,5-difluoro-9-methyl-2¹H-12-oxa-3,10-diaza-4(4,2)-pyridina-2(3,5)-pyrazola-1(1,3)-cyclopentanacyclododecaphan-6-en-11-one (compound 167); (1¹S,1³R,2⁴Z,9R)-5,5-difluoro-9-methyl-2¹H-12-oxa-3,10-diaza-4(4,2)-pyridina-2(3,5)-pyrazola-1(1,3)-cyclopentanacyclododecaphan-6-en-11-one (compound 168); (1¹S,1³R,7¹R,7³S,Z)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one (compound 169) and (1¹S,1³R,7¹S,7³R,Z)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one (compound 170) (all configurations are randomly assigned)

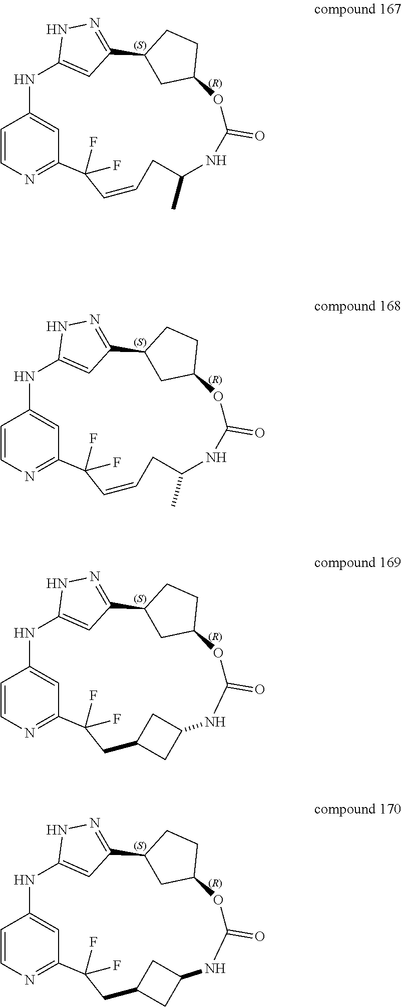

Step 1: Preparation of tert-butyl ((1r,3r)-3-(methoxy(methyl)carbamoyl)cyclobutyl)carbamate

To a solution of (1r,3r)-3-((tert-butoxycarbonyl)amino)cyclobutane-1-carboxylic acid (5.2 g, 24.16 mmol, 1 eq) in DCM (100 mL) was added HATU (13.78 g, 36.24 mmol, 1.5 eq) and DIEA (9.37 g, 72.48 mmol, 12.62 mL, 3 eq). After the mixture was stirred at 25° C. for 1 hour, N,O-dimethylhydroxylamine HCl salt (3.53 g, 36.24 mmol, 1.5 eq, HCl) was added, and the resulting mixture was kept stirring at 25° C. for 2 hours under N₂ atmosphere. The mixture was then concentrated in vacuo, and the resulting residue was purified by column chromatography to afford tert-butyl ((1r,3r)-3-(methoxy(methyl)carbamoyl)cyclobutyl)carbamate (5.8 g, 92.9% yield) as a colorless oil. LCMS: 203.2 [M−55+H]⁺.

Step 2: Preparation of tert-butyl ((1r,3r)-3-(2-(4-chloropyridin-2-yl)acetyl)cyclobutyl)carbamate

To a solution of 4-chloro-2-methyl-pyridine (1.35 g, 10.58 mmol, 1 eq) in THF (9 mL) was added LDA (2 M, 7.93 mL, 1.5 eq) at 0° C., and the mixture was stirred for 0.5 hour at 0° C. under N₂ atmosphere. Then a solution of tert-butyl ((1r,3r)-3-(methoxy(methyl)carbamoyl)cyclobutyl)carbamate (4.10 g, 15.87 mmol, 1.5 eq) in THF (9 mL) was added dropwise at 0° C. After the completion of the addition, the mixture was stirred at 25° C. for 1 hour under N₂ atmosphere. The mixture was quenched with saturated aqueous NH₄Cl (100 mL) and extracted with EtOAc (100 mL×2). The combined organic layers were washed with brine (50 mL), dried over anhydrous Na₂SO₄, filtered, and the filtrate was concentrated in vacuo. The resulting residue was purified by column chromatography, and further by prep-HPLC (FA condition: column: Phenomenex luna C18 (250×70 mm, 10 um); mobile phase: [water (FA)-ACN]; gradient:30%-60% B over 19 min) to give tert-butyl ((1r,3r)-3-(2-(4-chloropyridin-2-yl)acetyl)cyclobutyl)carbamate (940 mg, 27.4% yield) as a yellow solid. LCMS: 325.2 [M+H]⁺, 269.2 [M−55+H]⁺.

Step 3: Preparation of tert-butyl ((1r,3r)-3-(2-(4-chloropyridin-2-yl)-2,2-difluoroacetyl)cyclobutyl)carbamate

To a solution of tert-butyl ((1r,3r)-3-(2-(4-chloropyridin-2-yl)acetyl)cyclobutyl)carbamate (900 mg, 2.77 mmol, 1 eq) in DMF (20 mL) was added selectfluor (2.94 g, 8.31 mmol, 3 eq) and DBU (1.27 g, 8.31 mmol, 1.25 mL, 3 eq), and the mixture was stirred at 25° C. for 2 hours under N₂ atmosphere. After the completion of the reaction, the mixture was diluted with H₂O (50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by column chromatography to afford tert-butyl ((1r,3r)-3-(2-(4-chloropyridin-2-yl)-2,2-difluoroacetyl)cyclobutyl)carbamate (702 mg, 70.2% yield) as a white solid. LCMS: 305.1 [M−55+H]⁺.

Step 4: Preparation of tert-butyl ((1R,3r)-3-((R)-2-(4-chloropyridin-2-yl)-2,2-difluoro-1-hydroxyethyl)cyclobutyl)carbamate

To a solution of tert-butyl ((1r,3r)-3-(2-(4-chloropyridin-2-yl)-2,2-difluoroacetyl)cyclobutyl)carbamate (702 mg, 1.95 mmol, 1 eq) in MeOH (17 mL) was added NaBH₄ (147 mg, 3.89 mmol, 2 eq), and the mixture was stirred at 25° C. for 1 hour under N₂ atmosphere. After the completion of the reaction, the mixture was quenched with H₂O (100 mL) and extracted with EtOAc (100 mL×2). The combined organic layers were washed with brine (50 mL), dried over anhydrous Na₂SO₄, filtered, and the filtrate was concentrated in vacuo. The desired tert-butyl ((1R,3r)-3-((R)-2-(4-chloropyridin-2-yl)-2,2-difluoro-1-hydroxyethyl)cyclobutyl)carbamate (700 mg, 1.93 mmol) was obtained as a white solid, which was used into the next step without further purification. LCMS: 307.0 [M−55+H]⁺.

Step 5: Preparation of tert-butyl ((1R,3r)-3-((R)-2-(4-chloropyridin-2-yl)-2,2-difluoro-1-(((methylthio)carbonothioyl)oxy)ethyl)cyclobutyl)carbamate

To a solution of tert-butyl ((1R,3r)-3-((R)-2-(4-chloropyridin-2-yl)-2,2-difluoro-1-hydroxyethyl)cyclobutyl)carbamate (700 mg, 1.93 mmol, 1 eq) in THF (18 mL) was added dropwise CS₂ (2.93 g, 38.58 mmol, 0.26 mL, 20 eq) and NaOH (5 M, 3.09 mL, 8 eq) at 0° C. under N₂ atmosphere. After the completion of the addition, the mixture was warmed to 25° C. and kept stirring at that temperature for 40 minutes. The mixture was then cooled to 0° C. and CH₃I (6.85 g, 48.24 mmol, 25 eq) was added dropwise. The reaction was stirred at 0° C. for an additional 20 min, quenched with saturated aqueous NH₄Cl (50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by column chromatography to afford tert-butyl ((1R,3r)-3-((R)-2-(4-chloropyridin-2-yl)-2,2-difluoro-1-(((methylthio)carbonothioyl)oxy)ethyl)cyclobutyl)carbamate (870 mg, 99.5% yield) as a yellow oil. LCMS: 397.0 [M−55+H]⁺.

Step 6: Preparation of tert-butyl ((1r,3r)-3-(2-(4-chloropyridin-2-yl)-2,2-difluoroethyl)cyclobutyl)carbamate

To a solution of tert-butyl ((1R,3r)-3-((R)-2-(4-chloropyridin-2-yl)-2,2-difluoro-1-(((methylthio)carbonothioyl)oxy)ethyl)cyclobutyl)carbamate (870 mg, 1.92 mmol, 1 eq) in toluene (18 mL) was added AlBN (157.69 mg, 0.96 mmol, 0.5 eq) and bis(trimethylsilyl)silyl-trimethyl-silane (955.19 mg, 3.84 mmol, 1.19 mL, 2 eq), and the mixture was stirred at 110° C. for 1 hour under N₂ atmosphere. The mixture was cooled to room temperature and concentrated in vacuo. The resulting residue was purified by column chromatography and further by SFC (condition: column: DAICEL CHIRALPAK IG 250 mm×30 mm×5 um; mobile phase: [CO₂-MeOH (0.1% NH₃·H₂O)]; B %:25%, isocratic elution mode) to give tert-butyl ((1r,3r)-3-(2-(4-chloropyridin-2-yl)-2,2-difluoroethyl)cyclobutyl)carbamate (529 mg, 79.42% yield) as a yellow oil. LCMS: 347.3 [M+H]⁺, 291.3 [M−55+H]⁺; 1H NMR (400 MHz, DMSO-d₆) δ 8.65 (t, J=5.2 Hz, 1H), 7.82-7.70 (m, 2H), 4.03-3.86 (m, 1H), 2.50-2.19 (m, 5H), 1.98-1.90 (m, 2H), 1.37-1.34 (m, 9H) ppm.

Step 7: Preparation of tert-butyl ((1S,3r)-3-(2-(4-((1-(tert-butyl)-3-((1S,3R)-3-((tert-butyldimethylsilyl)oxy)cyclopentyl)-1H-pyrazol-5-yl)amino)pyridin-2-yl)-2,2-difluoroethyl)cyclobutyl)carbamate

A mixture of tert-butyl ((1r,3r)-3-(2-(4-chloropyridin-2-yl)-2,2-difluoroethyl)cyclobutyl)carbamate (300 mg, 0.86 mmol, 1 eq), 1-(tert-butyl)-3-((1S,3R)-3-((tert-butyldimethylsilyl)oxy)cyclopentyl)-1H-pyrazol-5-amine (321.22 mg, 0.95 mmol, 1.1 eq), Xantphos (100.11 mg, 0.17 mmol, 0.2 eq), Pd₂(dba)₃ (79.21 mg, 0.086 mmol, 0.1 eq) and Cs₂CO₃ (845.55 mg, 2.60 mmol, 3 eq) in dioxane (6 mL) was degassed and back-filled with N₂ for three times, and stirred at 90° C. for 12 hours under N₂ atmosphere. After the completion of the reaction, the mixture was cooled to room temperature. The mixture was filtered through a pad of the Celite, rinsed with dioxane (10 mL) and the filtrate was concentrated in vacuo. The resulting residue was purified by column chromatography to afford tert-butyl ((1S,3r)-3-(2-(4-((1-(tert-butyl)-3-((1S,3R)-3-((tert-butyldimethylsilyl)oxy)cyclopentyl)-1H-pyrazol-5-yl)amino)pyridin-2-yl)-2,2-difluoroethyl)cyclobutyl)carbamate (427 mg, 76.2% yield) as a brown oil. LCMS: 648.4 [M+H]⁺.

Step 8: Preparation of tert-butyl (2-(2-((1r,3S)-3-((tert-butoxycarbonyl)amino)cyclobutyl)-1,1-difluoroethyl)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((1S,3r)-3-(2-(4-((1-(tert-butyl)-3-((1S,3R)-3-((tert-butyldimethylsilyl)oxy)cyclopentyl)-1H-pyrazol-5-yl)amino)pyridin-2-yl)-2,2-difluoroethyl)cyclobutyl)carbamate (617 mg, 0.95 mmol, 1 eq) in DCM (31 mL) was added Boc₂O (353.32 mg, 1.62 mmol, 0.37 mL, 1.7 eq) and DMAP (116.34 mg, 0.95 mmol, 1 eq), and the reaction mixture was stirred at 25° C. for 2 hours under N₂ atmosphere. After the completion of the reaction, the mixture was concentrated in vacuo. The resulting residue was purified by column chromatography to afford tert-butyl (2-(2-((1r,3S)-3-((tert-butoxycarbonyl)amino)cyclobutyl)-1,1-difluoroethyl)pyridin-4-yl)(1-(tert-butyl)-3-((1S,3R)-3-((tert-butyldimethylsilyl)oxy)cyclopentyl)-1H-pyrazol-5-yl)carbamate (699 mg, 98.1% yield) as a yellow oil. LCMS: 748.4 [M+H]⁺.

Step 9: Preparation of tert-butyl (2-(2-((1r,3S)-3-((tert-butoxycarbonyl)amino)cyclobutyl)-1,1-difluoroethyl)pyridin-4-yl)(1-(tert-butyl)-3-((1S,3R)-3-hydroxycyclopentyl)-1H-pyrazol-5-yl)carbamate

To a solution of tert-butyl (2-(2-((1R,3S)-3-((tert-butoxycarbonyl)amino)cyclobutyl)-1,1-difluoroethyl)pyridin-4-yl)(1-(tert-butyl)-3-((1S,3R)-3-((tert-butyldimethylsilyl)oxy) cyclopentyl)-1H-pyrazol-5-yl)carbamate (699 mg, 0.93 mmol, 1 eq) in THF (14 mL) was added TBAF (1 M, 5.61 mL, 6 eq), and the reaction mixture was stirred at 25° C. for 4 hours under N₂ atmosphere. After the completion of the reaction, the mixture was quenched with H₂O (50 mL) and extracted with EtOAc (50 mL×2). The combined organic layers were washed with brine (50 mL), dried over anhydrous Na₂SO₄, filtered, and the filtrate was concentrated in vacuo. The desired tert-butyl (2-(2-((1r,3S)-3-((tert-butoxycarbonyl)amino)cyclobutyl)-1,1-difluoroethyl)pyridin-4-yl)(1-(tert-butyl)-3-((1S,3R)-3-hydroxycyclopentyl)-1H-pyrazol-5-yl)carbamate (590 mg, 0.92 mmol) was obtained as a yellow oil, which was used into the next step without further purification. LCMS: 634.4 [M+H]⁺.

Step 10: Preparation of tert-butyl (2-(2-((1r,3S)-3-((tert-butoxycarbonyl)amino)cyclobutyl)-1,1-difluoroethyl)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(2-((1r,3S)-3-((tert-butoxycarbonyl)amino)cyclobutyl)-1,1-difluoroethyl)pyridin-4-yl)(1-(tert-butyl)-3-((1S,3R)-3-hydroxycyclopentyl)-1H-pyrazol-5-yl)carbamate (580 mg, 0.91 mmol, 1 eq), (4-nitrophenyl) carbonochloridate (553.39 mg, 2.75 mmol, 3 eq) and DMAP (111.80 mg, 0.91 mmol, 1 eq) in DCM (14 mL) was added pyridine (434.34 mg, 5.49 mmol, 6 eq) dropwise, and the reaction mixture was stirred at 25° C. for 0.5 hours under N₂ atmosphere. After the completion of the reaction, the mixture was filtered, and the filtrate was concentrated in vacuo. The resulting residue was purified by column chromatography to afford tert-butyl (2-(2-((1r,3S)-3-((tert-butoxycarbonyl)amino)cyclobutyl)-1,1-difluoroethyl)pyridin-4-yl)(1-(tert-butyl)-3-((1S,3R)-3-(((4-nitrophenoxy)carbonyl)oxy)cyclopentyl)-1H-pyrazol-5-yl)carbamate (730 mg, 99.8% yield) as a colorless oil. LCMS: 799.4 [M+H]⁺.

Step 11: Preparation of (1R,3S)-3-(5-((2-(2-((1r,3S)-3-aminocyclobutyl)-1,1-difluoroethyl)pyridin-4-yl)amino)-1-(tert-butyl)-1H-pyrazol-3-yl)cyclopentyl (4-nitrophenyl) carbonate

To a solution of tert-butyl (2-(2-((1r,3S)-3-((tert-butoxycarbonyl)amino)cyclobutyl)-1,1-difluoroethyl)pyridin-4-yl)(1-(tert-butyl)-3-((1S,3R)-3-(((4-nitrophenoxy)carbonyl)oxy)cyclopentyl)-1H-pyrazol-5-yl)carbamate (730 mg, 0.91 mmol, 1 eq) in DCM (21 mL) was added TFA (7 mL), and the mixture was stirred at 25° C. for 12 hours under N₂ atmosphere. The mixture was then concentrated in vacuo to afford (1R,3S)-3-(5-((2-(2-((1r,3S)-3-aminocyclobutyl)-1,1-difluoroethyl)pyridin-4-yl)amino)-1-(tert-butyl)-1H-pyrazol-3-yl)cyclopentyl (4-nitrophenyl) carbonate (547 mg, 0.91 mmol) as a yellow oil, which was used into the next step without further purification. LCMS: 599.2 [M+H]⁺.

Step 12: Preparation of (1¹S,1³R,7¹R,7³S,Z)-2¹-(tert-butyl)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one

To a solution of (1R,3S)-3-(5-((2-(2-((1r,3S)-3-aminocyclobutyl)-1,1-difluoroethyl)pyridin-4-yl)amino)-1-(tert-butyl)-1H-pyrazol-3-yl)cyclopentyl (4-nitrophenyl) carbonate (300 mg, 0.50 mmol, 1 eq) in THF (150 mL) was added DIEA (0.32 g, 2.50 mmol, 5 eq), and the mixture was stirred at 25° C. for 12 hours under N₂ atmosphere. After the completion of the reaction, the mixture was concentrated in vacuo to afford (1¹S,1³R,7¹R,7³S,Z)-2¹-(tert-butyl)-5,5-difluoro-2¹H-1 0-oxa-3,8-diaza-4(4,2)-pyridina-2(3,5)-pyrazola-1 (1,3)-cyclopentana-7(1,3)-cyclobutanacyclodecaphan-9-one (230 mg) as a yellow oil, which was used into the next step without further purification. LCMS: 460.4 [M+H]⁺.

Step 13: Preparation of (1¹S,1³R,7¹R,7³S,Z)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one

A mixture of (1¹S,1³R,7¹R,7³S,Z)-2¹-(tert-butyl)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one (220 mg, 0.47 mmol, 1 eq) in formic acid (4.4 mL) was stirred at 100° C. for 0.5 hour under N₂ atmosphere. After the completion of the reaction, the mixture was cooled to room temperature and concentrated in vacuo. The resulting residue was purified by prep-HPLC (FA condition column: Phenomenex luna C18 150×25 mm×10 um; mobile phase: [water (FA)-ACN]; gradient:7%-37% B over 10 min) and fractions of a major peak that contains 4 inseparable isomers were collected. The fractions were evaporated to afford the desired (1¹S,1³R,7¹R,7³S,Z)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one and other isomers (90 mg, 46.6% yield) as a white solid LCMS: 404.3 [M+H]⁺.

Step 14: Isolation of (1¹S,1³R,2⁴Z,9S)-5,5-difluoro-9-methyl-2¹H-12-oxa-3,10-diaza-4(4,2)-pyridina-2(3,5)-pyrazola-1(1,3)-cyclopentanacyclododecaphan-6-en-11-one (compound 167); (1¹S,1³R,2⁴Z,9R)-5,5-difluoro-9-methyl-2¹H-12-oxa-3,10-diaza-4(4,2)-pyridina-2(3,5)-pyrazola-1(1,3)-cyclopentanacyclododecaphan-6-en-11-one (compound 168); (1¹S,1³R,7¹R,7³S,Z)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one (compound 169) and (1¹S,1³R,7¹S,7³R,Z)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one(compound 170) (all configurations are randomly assigned)

The mixture from step 13 was further purified by SFC (condition: column: DAICEL CHIRALPAK AS (250 mm×30 mm, 10 um); mobile phase: [CO₂-MeOH (0.1% NH₃·H₂O)]; B %:30%, isocratic elution mode) to afford (1¹S,1³R,2⁴Z,9S)-5,5-difluoro-9-methyl-2¹H-12-oxa-3,10-diaza-4(4,2)-pyridina-2(3,5)-pyrazola-1(1,3)-cyclopentanacyclododecaphan-6-en-11-one (compound 167) (7.48 mg, 3.8% yield) as a light-yellow solid, (1¹S,1³R,2⁴Z,9R)-5,5-difluoro-9-methyl-2¹H-12-oxa-3,10-diaza-4(4,2)-pyridina-2(3,5)-pyrazola-1(1,3)-cyclopentanacyclododecaphan-6-en-11-one (compound 168) (4.62 mg, 2.3% yield) as a white solid, (1¹S,1³R,7¹R,7³S,Z)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one (compound 169) (35.6 mg, 18.3% yield) as a white solid, and (1¹S,1³R,7¹S,7³R,Z)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one(compound 170) (16.95 mg, 8.7% yield) as a light yellow solid.

(1¹S,1³R,2⁴Z,9S)-5,5-difluoro-9-methyl-2¹H-12-oxa-3,10-diaza-4(4,2)-pyridina-2(3,5)-pyrazola-1(1,3)-cyclopentanacyclododecaphan-6-en-11-one (compound 167) LCMS: 404.2 [M+H]⁺; ¹H NMR (400 MHz, DMSO-d₆) δ 12.43-12.06 (m, 1H), 9.37-9.03 (m, 1H), 8.17 (br d, J=5.25 Hz, 1H), 7.06-6.88 (m, 2H), 6.83 (br d, J=4.00 Hz, 1H), 6.02-5.87 (m, 2H), 5.84 (s, 1H), 5.09-4.85 (m, 1H,) 3.64 (br d, J=2.00 Hz, 1H), 3.20 (br d, J=6.88 Hz, 1H), 2.47-2.37 (m, 1H), 2.36-2.27 (m, 1H), 2.13-1.95 (m, 2H), 1.83-1.64 (m, 4H), 1.09-0.95 (m, 3H) ppm.

(1¹S,1³R,2⁴Z,9R)-5,5-difluoro-9-methyl-2¹H-12-oxa-3,10-diaza-4(4,2)-pyridina-2(3,5)-pyrazola-1(1,3)-cyclopentanacyclododecaphan-6-en-11-one (compound 168) LCMS: 404.2 [M+H]⁺; ¹H NMR (400 MHz, DMSO-d₆) δ 12.41-12.06 (m, 1H), 9.35-9.03 (m, 1H), 8.17 (br d, J=4.38 Hz, 1H), 6.95 (s, 1H), 6.83 (br s, 2H), 6.15-5.76 (m, 3H), 5.15-4.78 (m, 1H), 3.53-3.42 (m, 1H), 3.26-3.14 (m, 1H), 2.38-2.13 (m, 3H), 2.11-1.99 (m, 1H), 1.89-1.62 (m, 4H), 1.08-1.01 (m, 3H) ppm.

(1¹S,1³R,7¹R,7³S,Z)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one (compound 169) LCMS: 404.2 [M+H]⁺; ¹H NMR (400 MHz, DMSO-d₆) δ 12.54-12.00 (m, 1H), 9.20-8.84 (m, 1H), 8.19 (d, J=5.50 Hz, 1H), 7.72-6.89 (m, 1H), 6.88-6.71 (m, 2H), 6.07-5.68 (m, 1H), 4.91 (br s, 1H), 3.85-3.53 (m, 1H), 3.19 (br d, J=7.00 Hz, 1H), 2.47-2.37 (m, 1H), 2.37-1.99 (m, 4H), 1.99-1.61 (m, 8H) ppm.

(1¹S,1³R,7¹S,7³R,Z)-5,5-difluoro-2¹H-10-oxa-3,8-diaza-4(4,2)-pyridina-2(3,5)-pyrazola-1(1,3)-cyclopentana-7(1,3)-cyclobutanacyclodecaphan-9-one(compound 170) LCMS: 404.2 [M+H]⁺; ¹H NMR (400 MHz, DMSO-d₆) δ 12.62-12.11 (m, 1H), 8.94 (br s, 1H), 8.29-8.07 (m, 1H), 7.08 (br s, 1H), 6.90-6.64 (m, 2H), 5.92 (s, 1H), 5.01 (br s, 1H), 3.51-3.44 (m, 1H), 3.27-3.10 (m, 1H), 2.48-2.38 (m, 1H), 2.32-2.00 (m, 5H), 1.84 (br d, J=8.76 Hz, 1H), 1.77-1.56 (m, 3H), 1.55-1.21 (m, 3H) ppm.

Example 35: synthesis of (1¹S,1³R,9S,Z)-9-methyl-2¹H-5,12-dioxa-3,10-diaza-4(4,2)-pyridina-2(3,5)-pyrazola-1(1,3)-cyclopentanacyclododecaphan-11-one-6,6-d₂ (compound 173)

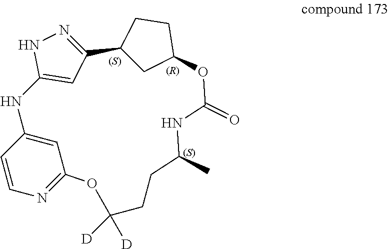

Step 1: Preparation of tert-butyl (S)-(5-hydroxypentan-2-yl-5,5-d₂)carbamate

To a solution of ethyl (S)-4-((tert-butoxycarbonyl)amino)pentanoate (1.25 g, 5.10 mmol, 1 eq) in THF (10 mL) was added LiAlD4 (278 mg, 6.62 mmol, 1.3 eq) at 0° C., and the mixture was then warmed to 25° C. and kept stirring at that temperature for 1 hour under N₂ atmosphere. After the starting material was consumed, the mixture was carefully poured into water (50 mL) and extracted with EtOAc (50 mL×3). The organic phase was dried over Na₂SO₄, filtered and the filtrate was concentrated to afford tert-butyl (S)-(5-hydroxypentan-2-yl-5,5-d₂)carbamate (0.9 g, 86% yield) as a yellow oil. ¹H NMR (400 MHz, CHLOROFORM-d): δ 4.60 (br d, J=6.6 Hz, 1H), 3.58 (br s, 1H), 3.04 (br s, 1H), 1.53-1.48 (m, 2H), 1.45-1.39 (m, 2H), 1.36 (s, 9H), 1.05 (d, J=6.6 Hz, 3H) ppm

Step 2: Preparation of tert-butyl (S)-(5-((4-bromopyridin-2-yl)oxy)pentan-2-yl-5,5-d₂)carbamate

To a solution of tert-butyl (S)-(5-hydroxypentan-2-yl-5,5-d₂)carbamate (0.27 g, 0.789 mmol, 1 eq) and t-BuOK (159.4 mg, 1.42 mmol, 1.8 eq) in THF (3 mL) was added 4-bromo-2-fluoro-pyridine (208.3 mg, 1.18 mmol, 1.5 eq), and the mixture was stirred at 25° C. for 3 hours under N₂ atmosphere. After the reaction was completed, the mixture was quenched with H₂O (50 mL) and extracted with EtOAc (70 mL×2). The combined organic layers were washed with brine (50 mL×2), dried over anhydrous Na₂SO₄, filtered and the filtrate was concentrated in vacuo. The resulting residue was purified by column chromatography to afford tert-butyl (S)-(5-((4-bromopyridin-2-yl)oxy)pentan-2-yl-5,5-d₂)carbamate (0.21 g, 70.2% yield) as a white solid. ¹H NMR (400 MHz, CHLOROFORM-d): δ 7.80 (br d, J=6.0 Hz, 1H), 6.92 (br d, J=6.0 Hz, 1H), 6.85 (s, 1H), 4.39 (s, 1H), 3.63 (s, 1H), 1.72-1.68 (m, 2H), 1.48-1.47 (m, 2H), 1.46-1.44 (m, 9H), 1.07-1.05 (m, 3H) ppm.

Step 3: Preparation of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)pentan-2-yl-5,5-d₂)carbamate

A mixture of tert-butyl (S)-(5-((4-bromopyridin-2-yl)oxy)pentan-2-yl-5,5-d₂)carbamate (0.2 g, 0.554 mmol, 1 eq), 1-(tert-butyl)-3-((1S,3R)-3-((tert-butyldimethylsilyl)oxy)cyclopentyl)-1H-pyrazol-5-amine (186.9 mg, 0.554 mmol, 1 eq), Pd₂(dba)₃ (76 mg, 0.083 mmol, 0.15 eq), XantPhos (39.6 mg, 0.083 mmol, 0.15 eq) and Cs₂CO₃ (541.1 mg, 1.66 mmol, 3 eq) in dioxane (4 mL) was degassed and backfilled with N₂ for 3 times, and the mixture was then stirred at 90° C. for 12 hours under N₂ atmosphere. After the completion of the reaction, the mixture was cooled to room temperature. The reaction mixture was filtered, rinsed with ethyl acetate (15 mL), and the filtrate was concentrated under reduced pressure. The resulting residue was purified by column chromatography to afford tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)pentan-2-yl-5,5-d₂)carbamate (270 mg, 79% yield) as a white solid. LCMS: 618.6 [M+H]⁺.

Step 4: Preparation of tert-butyl (2-(((S)-4-((tert-butoxycarbonyl)amino)pentyl-1,1-d₂)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)pentan-2-yl-5,5-d₂)carbamate (260 mg, 0.421 mmol, 1 eq) in DCM (3 mL) was added Boc₂O (137.7 mg, 0.631 mmol, 1.5 eq) and DMAP (51.4 mg, 0.421 mmol, 1 eq), and the mixture was stirred at 25° C. for 3 hours under N₂ atmosphere. After the completion of the reaction, the reaction mixture was concentrated under reduced pressure. The resulting residue was purified by column chromatography to afford tert-butyl (2-(((S)-4-((tert-butoxycarbonyl)amino)pentyl-1,1-d₂)oxy)pyridin-4-yl)(1-(tert-butyl)-3-((1S,3R)-3-((tert-butyldimethylsilyl)oxy)cyclopentyl)-1H-pyrazol-5-yl)carbamate (230 mg, 76.1% yield) as a colorless oil. LCMS: 718.7 [M+H]⁺.

Step 5: Preparation of tert-butyl (2-(((S)-4-((tert-butoxycarbonyl)amino)pentyl-1,1-d₂)oxy)pyridin-4-yl)(1-(tert-butyl)-3-((1S,3R)-3-hydroxycyclopentyl)-1H-pyrazol-5-yl)carbamate

To a solution of tert-butyl (2-(((S)-4-((tert-butoxycarbonyl)amino)pentyl-1,1-d₂)oxy)pyridin-4-yl)(1-(tert-butyl)-3-((1S,3R)-3-((tert-butyldimethylsilyl)oxy)cyclopentyl)-1H-pyrazol-5-yl)carbamate (220 mg, 0.306 mmol, 1 eq) in THF (3 mL) was added TBAF (1 M, 1.84 mL, 6 eq), and the mixture was stirred at 20° C. for 1 hour under N₂ atmosphere. The mixture was then concentrated in vacuo to afford tert-butyl (2-(((S)-4-((tert-butoxycarbonyl)amino)pentyl-1,1-d₂)oxy)pyridin-4-yl)(1-(tert-butyl)-3-((1S,3R)-3-hydroxycyclopentyl)-1H-pyrazol-5-yl)carbamate (230 mg, 0.38 mmol) as a yellow oil, which was used into the next step without further purification. LCMS: 604.6 [M+H]⁺.

Step 6: Preparation of tert-butyl (2-(((S)-4-((tert-butoxycarbonyl)amino)pentyl-1,1-d₂)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(((S)-4-((tert-butoxycarbonyl)amino)pentyl-1,1-d₂)oxy)pyridin-4-yl)(1-(tert-butyl)-3-((1S,3R)-3-hydroxycyclopentyl)-1H-pyrazol-5-yl)carbamate (220 mg, 0.364 mmol, 1 eq), (4-nitrophenyl) carbonochloridate (220.33 mg, 1.09 mmol, 3 eq) and Pyridine (172.93 mg, 2.19 mmol, 6 eq) in DCM (4 mL) was added DMAP (44.51 mg, 0.36 mmol, 1 eq), and the mixture was stirred at 20° C. for 2 hours under N₂ atmosphere. After the completion of the reaction, the mixture was filtered, and the filtrate was concentrated in vacuo. The resulting residue was purified by column chromatography to afford tert-butyl (2-(((S)-4-((tert-butoxycarbonyl)amino)pentyl-1,1-d₂)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (240 mg, 85.7% yield) as a colorless oil. LCMS: 769.6 [M+H]⁺.

Step 7: Preparation of (1R,3S)-3-(5-((2-(((S)-4-aminopentyl-1,1-d₂)oxy)pyridin-4-yl)amino)-1-(tert-butyl)-1H-pyrazol-3-yl)cyclopentyl (4-nitrophenyl) carbonate

To a solution of tert-butyl (2-(((S)-4-((tert-butoxycarbonyl)amino)pentyl-1,1-d₂)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (220 mg, 0.286 mmol, 1 eq) in DCM (3 mL) was added TFA (2.30 g, 20.19 mmol, 1.5 mL, 70.57 eq), and the mixture was stirred at 20° C. for 12 hours under N₂ atmosphere. The mixture was then concentrated in vacuo to afford (1R,3S)-3-(5-((2-(((S)-4-aminopentyl-1,1-d₂)oxy)pyridin-4-yl)amino)-1-(tert-butyl)-1H-pyrazol-3-yl)cyclopentyl (4-nitrophenyl) carbonate (180 mg, 0.317 mmol) as a yellow oil, which was used into the next step without further purification. LCMS: 569.5 [M+H]⁺.

Step 8: Preparation of (1¹S,1³R,9S,Z)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-11-one-6,6-d₂

To a solution of (1R,3S)-3-(5-((2-(((S)-4-aminopentyl-1,1-d₂)oxy)pyridin-4-yl)amino)-1-(tert-butyl)-1H-pyrazol-3-yl)cyclopentyl (4-nitrophenyl) carbonate (170 mg, 0.299 mmol, 1 eq) in THF (500 mL) was added DIEA (38.64 mg, 0.299 mmol, 52.07 μL, 1 eq), and the mixture was stirred at 20° C. for 6 hours under N₂ atmosphere. The mixture was then concentrated in vacuo to afford (1¹S,1³R,9S,Z)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-11-one-6,6-d₂ (130 mg, 0.303 mmol) as a yellow oil, which was used in the next step without further purification. LCMS: 430.3 [M+H]⁺.

Step 9: Preparation of (1¹S,1³R,9S,Z)-9-methyl-2¹H-5,12-dioxa-3,10-diaza-4(4,2)-pyridina-2(3,5)-pyrazola-1(1,3)-cyclopentanacyclododecaphan-11-one-6,6-d₂

A solution of (1¹S,1³R,9S,Z)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-11-one-6,6-d₂ (120 mg, 0.279 mmol, 1 eq) in formic acid (1 mL) was stirred at 100° C. for 1 hour under N₂ atmosphere. After the completion of the reaction, the mixture was cooled to room temperature and concentrated in vacuo. The resulting residue was purified by prep-HPLC {column: Phenomenex luna C18 150*25 mm*10 um; mobile phase: [water(FA)-ACN]; gradient:10%-40% B over 10 min} to afford (1¹S,1³R,9S,Z)-9-methyl-2¹H-5,12-dioxa-3,10-diaza-4(4,2)-pyridina-2(3,5)-pyrazola-1(1,3)-cyclopentanacyclododecaphan-11-one-6,6-d₂ (66 mg, 63.4% yield) as a white solid. LCMS: 374.3 [M+H]⁺; ¹H NMR (400 MHz, DMSO-d₆) δ ppm 12.70-12.23 (m, 1H), 10.05 (s, 1H), 7.86 (d, J=6.64 Hz, 1H), 6.96 (d, J=8.64 Hz, 1H), 6.64 (d, J=6.38 Hz, 1H), 6.45 (s, 1H), 6.15 (s, 1H), 5.06 (d, J=4.00 Hz, 1H), 3.73-3.62 (m, 1H), 3.26-3.22 (m, 1H), 2.44-2.33 (m, 1H), 2.18-2.02 (m, 1H), 1.89-1.71 (m, 6H), 1.58 (dd, J=9.88, 3.25 Hz, 1H), 1.50-1.38 (m, 1H), 1.03 (d, J=6.64 Hz, 3H) ppm.

Example 36: synthesis of (1¹S,1³R,9S,Z)-4⁵-fluoro-9-methyl-2¹H-5,12-dioxa-3,10-diaza-4(4,2)-pyridina-2(3,5)-pyrazola-1(1,3)-cyclopentanacyclododecaphan-11-one (compound 174)

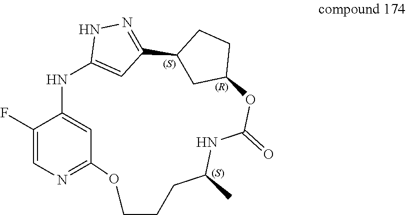

Step 1: Preparation of tert-butyl (S)-(5-((4-bromo-5-fluoropyridin-2-yl)oxy)pentan-2-yl)carbamate

A mixture of 4-bromo-5-fluoro-pyridin-2-ol (300.0 mg, 1.56 mmol, 1.1 eq), tert-butyl (S)-(5-hydroxypentan-2-yl)carbamate (288.77 mg, 1.42 mmol, 1 eq), DIAD (344.70 mg, 1.70 mmol, 1.2 eq) and PPh₃ (447.11 mg, 1.70 mmol, 1.2 eq) in THF (6 mL) was stirred at 25° C. for 3 hours under N₂ atmosphere. After the reaction was completed, the mixture was diluted with H₂O (50 mL) and extracted with EtOAc (100 mL×2). The combined organic layers were washed with brine (50 mL×2), dried over anhydrous Na₂SO₄, filtered, and the filtrate was concentrated in vacuo. The resulting residue was purified by column chromatography to give tert-butyl (S)-(5-((4-bromo-5-fluoropyridin-2-yl)oxy)pentan-2-yl)carbamate (250 mg, 46.6% yield) as a yellow oil. LCMS: 320.9, 322.9 [M+H]⁺.

Step 2: Preparation of tert-butyl ((S)-5-((4-((1-(tert-butyl)-3-((1S,3R)-3-((tert-butyldimethylsilyl)oxy)cyclopentyl)-1H-pyrazol-5-yl)amino)-5-fluoropyridin-2-yl)oxy)pentan-2-yl)carbamate

A mixture of tert-butyl (S)-(5-((4-bromo-5-fluoropyridin-2-yl)oxy)pentan-2-yl)carbamate (200.00 mg, 0.53 mmol, 1 eq), 1-(tert-butyl)-3-((1S,3R)-3-((tert-butyldimethylsilyl)oxy)cyclopentyl)-1H-pyrazol-5-amine (196.86 mg, 0.58 mmol, 1.1 eq), Pd₂(dba)₃ (48.55 mg, 0.53 mmol, 0.1 eq), Xantphos (36.81 mg, 0.63 mmol, 0.12 eq) and Cs₂CO₃ (518.21 mg, 1.59 mmol, 3 eq) in dioxane (3 mL) was stirred at 90° C. under N₂ atmosphere for 3 hours. After the completion of the reaction, the mixture was cooled to room temperature. The mixture was diluted with H₂O (50 mL) and extracted with EtOAc (100 mL×2). The combined organic layers were washed with brine (50 mL×2), dried over anhydrous Na₂SO₄, filtered, and the filtrate was concentrated in vacuo. The desired tert-butyl ((S)-5-((4-((1-(tert-butyl)-3-((1S,3R)-3-((tert-butyldimethylsilyl)oxy)cyclopentyl)-1H-pyrazol-5-yl)amino)-5-fluoropyridin-2-yl)oxy)pentan-2-yl)carbamate (427 mg, crude) was obtained as a yellow oil, which was used in the next step without further purification. LCMS: 634.5 [M+H]⁺.

Step 3: Preparation of tert-butyl (2-(((S)-4-((tert-butoxycarbonyl)amino)pentyl)oxy)-5-fluoropyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-5-((4-((1-(tert-butyl)-3-((1S,3R)-3-((tert-butyldimethylsilyl)oxy)cyclopentyl)-1H-pyrazol-5-yl)amino)-5-fluoropyridin-2-yl)oxy)pentan-2-yl)carbamate (337.00 mg, 0.53 mmol, 1 eq) and Boc₂O (174.04 mg, 0.80 mmol, 1.5 eq) in DCM (3 mL) was added DMAP (32.47 mg, 0.27 mmol, 0.5 eq), and the mixture was stirred at 25° C. for 3 hours under N₂ atmosphere. After the completion of the reaction, the mixture was diluted with H₂O (50 mL) and extracted with EtOAc (100 mL×2). The combined organic layers were washed with brine (50 mL×2), dried over anhydrous Na₂SO₄, filtered, and the filtrate was concentrated in vacuo. The resulting residue was purified by column chromatography to give tert-butyl (2-(((S)-4-((tert-butoxycarbonyl)amino)pentyl)oxy)-5-fluoropyridin-4-yl)(1-(tert-butyl)-3-((1S,3R)-3-((tert-butyldimethylsilyl)oxy)cyclopentyl)-1H-pyrazol-5-yl)carbamate (390 mg, 99.9% yield) as a yellow oil. LCMS: 735.1 [M+H]⁺.

Step 4: Preparation of tert-butyl (2-(((S)-4-((tert-butoxycarbonyl)amino)pentyl)oxy)-5-fluoropyridin-4-yl)(1-(tert-butyl)-3-((1S,3R)-3-hydroxycyclopentyl)-1H-pyrazol-5-yl)carbamate

A mixture of tert-butyl (2-(((S)-4-((tert-butoxycarbonyl)amino)pentyl)oxy)-5-fluoropyridin-4-yl)(1-(tert-butyl)-3-((1S,3R)-3-((tert-butyldimethylsilyl)oxy)cyclopentyl)-1H-pyrazol-5-yl)carbamate (340.00 mg, 0.46 mmol, 1 eq) and TBAF (121.11 mg, 0.46 mmol, 1 eq) in THF (5 mL) was stirred at 25° C. for 1 hour under N₂ atmosphere. The reaction mixture was then quenched with saturated aqueous NH₄Cl (50 mL) and extracted with EtOAc (20 mL×3). The combined organic layers were washed with brine (50 mL), dried over Na₂SO₄, filtered, and the filtrate was concentrated under reduced pressure to give tert-butyl (2-(((S)-4-((tert-butoxycarbonyl)amino)pentyl)oxy)-5-fluoropyridin-4-yl)(1-(tert-butyl)-3-((1S,3R)-3-hydroxycyclopentyl)-1H-pyrazol-5-yl)carbamate (600 mg, crude) as a yellow oil, which was used in the next step without further purification. LCMS: 620.6 [M+H]⁺.

Step 5: Preparation of tert-butyl (2-(((S)-4-((tert-butoxycarbonyl)amino)pentyl)oxy)-5-fluoropyridin-4-yl)(1-(tert-butyl)-3-((1S,3R)-3-(((4-nitrophenoxy)carbonyl)oxy)cyclopentyl)-1H-pyrazol-5-yl)carbamate

The mixture of tert-butyl (2-(((S)-4-((tert-butoxycarbonyl)amino)pentyl)oxy)-5-fluoropyridin-4-yl)(1-(tert-butyl)-3-((1S,3R)-3-hydroxycyclopentyl)-1H-pyrazol-5-yl)carbamate (550 mg, 0.89 mmol, 1 eq), (4-nitrophenyl) carbonochloridate (536.62 mg, 2.66 mmol, 3 eq), DMAP (54.21 mg, 0.44 mmol, 0.5 eq) and Pyridine (421.17 mg, 5.32 mmol, 6 eq) in DCM (10 mL) was stirred at 25° C. for 3 hours under N₂ atmosphere. After the completion of the reaction, the mixture was diluted with H₂O (50 mL) and extracted with EtOAc (100 mL×2). The combined organic layers were washed with brine (50 mL×2), dried over anhydrous Na₂SO₄, filtered, and the filtrate was concentrated in vacuo. The resulting residue was purified by column chromatography to give tert-butyl (2-(((S)-4-((tert-butoxycarbonyl)amino)pentyl)oxy)-5-fluoropyridin-4-yl)(1-(tert-butyl)-3-((1S,3R)-3-(((4-nitrophenoxy)carbonyl)oxy)cyclopentyl)-1H-pyrazol-5-yl)carbamate (377 mg, 54.1% yield) as a yellow oil. LCMS: 785.4 [M+H]⁺.

Step 6: Preparation of (1R,3S)-3-(5-((2-(((S)-4-aminopentyl)oxy)-5-fluoropyridin-4-yl)amino)-1-(tert-butyl)-1H-pyrazol-3-yl)cyclopentyl (4-nitrophenyl) carbonate

The mixture of tert-butyl (2-(((S)-4-((tert-butoxycarbonyl)amino)pentyl)oxy)-5-fluoropyridin-4-yl)(1-(tert-butyl)-3-((1S,3R)-3-(((4-nitrophenoxy)carbonyl)oxy)cyclopentyl)-1H-pyrazol-5-yl)carbamate (327 mg, 0.41 mmol, 1 eq) and TFA (285.03 mg, 2.50 mmol, 6 eq) in DCM (2 mL) was stirred at 25° C. for 3 hours under N₂ atmosphere. After the completion of the reaction, the mixture was concentrated in vacuo to give (1R,3S)-3-(5-((2-(((S)-4-aminopentyl)oxy)-5-fluoropyridin-4-yl)amino)-1-(tert-butyl)-1H-pyrazol-3-yl)cyclopentyl (4-nitrophenyl) carbonate (243 mg, 99.7% yield) as a yellow oil, which was used in the next step without further purification.

Step 7: Preparation of (1¹S,1³R,9S,Z)-2¹-(tert-butyl)-4⁵-fluoro-9-methyl-2¹H-5,12-dioxa-3,10-diaza-4(4,2)-pyridina-2(3,5)-pyrazola-1(1,3)-cyclopentanacyclododecaphan-11-one

A mixture of (1R,3S)-3-(5-((2-(((S)-4-aminopentyl)oxy)-5-fluoropyridin-4-yl)amino)-1-(tert-butyl)-1H-pyrazol-3-yl)cyclopentyl (4-nitrophenyl) carbonate (193 mg, 0.33 mmol, 1 eq) and DIEA (128.00 mg, 0.99 mmol, 3 eq) in THF (193 mL) was stirred at 25° C. for 3 hours under N₂ atmosphere. After the completion of the reaction, the mixture was concentrated in vacuo to give (1¹S,1³R,9S,Z)-2¹-(tert-butyl)-4⁵-fluoro-9-methyl-2¹H-5,12-dioxa-3,10-diaza-4(4,2)-pyridina-2(3,5)-pyrazola-1(1,3)-cyclopentanacyclododecaphan-11-one (147 mg, 99.9% yield) as a yellow oil, which was used in the next step without further purification. LCMS: 446.4 [M+H]⁺.

Step 8: Preparation of (1¹S,1³R,9S,Z)-4⁵-fluoro-9-methyl-2¹H-5,12-dioxa-3,10-diaza-4(4,2)-pyridina-2(3,5)-pyrazola-1 (1,3)-cyclopentanacyclododecaphan-11-one

A mixture of (1¹S,1³R,9S,Z)-2¹-(tert-butyl)-4⁵-fluoro-9-methyl-2¹H-5,12-dioxa-3,10-diaza-4(4,2)-pyridina-2(3,5)-pyrazola-1(1,3)-cyclopentanacyclododecaphan-11-one (97 mg, 0.22 mmol, 1 eq) in HCOOH (3 mL) was stirred at 100° C. for 0.5 hour under N₂ atmosphere. After the completion of the reaction, the mixture was cooled to room temperature and concentrated. The resulting residue was purified by prep-HPLC {column: Phenomenex luna C18 150×25 mm×10 um; mobile phase: [water (FA)-ACN]; gradient:12%-32% B over 10 min} to give (1¹S,1³R,9S,Z)-4⁵-fluoro-9-methyl-2¹H-5,12-dioxa-3,10-diaza-4(4,2)-pyridina-2(3,5)-pyrazola-1(1,3)-cyclopentanacyclododecaphan-11-one (26.46 mg, 0.67 mmol, 31.0% yield, 99.3% purity) as a white solid. LCMS: 390.2 [M+H]⁺; ¹H NMR (400 MHz, DMSO-d₆) δ 12.58-11.95 (m, 1H), 8.93 (br s, 1H), 7.91 (br d, J=3.0 Hz, 1H), 6.90 (br d, J=8.9 Hz, 1H), 6.41 (d, J=6.1 Hz, 1H), 6.07 (s, 1H), 5.12-4.86 (m, 1H), 4.17-3.89 (m, 2H), 3.67-3.59 (m, 1H), 3.24-3.18 (m, 1H), 2.43-2.36 (m, 1H), 2.14-2.00 (m, 1H), 1.91-1.64 (m, 6H), 1.58-1.39 (m, 2H), 1.02 (d, J=6.4 Hz, 2H) ppm.

Example 37: synthesis of (1¹S,1³R,9S,Z)-4⁶-fluoro-9-methyl-2¹H-5,12-dioxa-3,10-diaza-4(4,2)-pyridina-2(3,5)-pyrazola-1(1,3)-cyclopentanacyclododecaphan-11-one (compound 178)

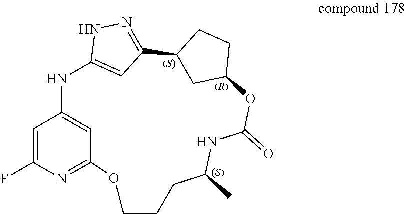

Step 1: Preparation of tert-butyl (S)-(5-((4-bromo-6-fluoropyridin-2-yl)oxy)pentan-2-yl)carbamate

A mixture of tert-butyl (S)-(5-hydroxypentan-2-yl)carbamate (191.43 mg, 0.94 mmol, 1 eq), 4-bromo-2,6-difluoro-pyridine (274 mg, 1.41 mmol, 1.5 eq) and t-BuOK (105.67 mg, 0.94 mmol, 1 eq) in THF (3 mL) was stirred at 25° C. for 3 hours under N₂ atmosphere. After the completion of the reaction, the mixture was poured into H₂O (40 mL) and extracted with EtOAc (20 mL×2). The combined organic layers were washed with H₂O (30 mL×2) and brine (30 mL), dried over anhydrous Na₂SO₄, filtered, and the filtrate was concentrated in vacuo. The resulting residue was purified by column chromatography to give tert-butyl (S)-(5-((4-bromo-6-fluoropyridin-2-yl)oxy)pentan-2-yl)carbamate (155 mg, 43.6% yield) as a yellow oil. LCMS: 277.2, 279.2 [M+H-100]⁺.

Step 2: Preparation of tert-butyl ((S)-5-((4-((1-(tert-butyl)-3-((1S,3R)-3-((tert-butyldimethylsilyl)oxy)cyclopentyl)-1H-pyrazol-5-yl)amino)-6-fluoropyridin-2-yl)oxy)pentan-2-yl)carbamate

A mixture of tert-butyl (S)-(5-((4-bromo-6-fluoropyridin-2-yl)oxy)pentan-2-yl)carbamate (105 mg, 0.28 mmol, 1 eq), 1-(tert-butyl)-3-((1S,3R)-3-((tert-butyldimethylsilyl)oxy)cyclopentyl)-1H-pyrazol-5-amine (103.35 mg, 0.31 mmol, 1.1 eq), Pd(dba)₂ (16.00 mg, 0.028 mmol, 0.1 eq), Xantphos (19.33 mg, 0.033 mmol, 0.12 eq) and Cs₂CO₃ (272.06 mg, 0.84 mmol, 3 eq) in dioxane (3 mL) was stirred at 90° C. under N₂ atmosphere for 3 hours. After the completion of the reaction, the mixture was cooled to room temperature. The mixture was then poured into H₂O (40 mL) and extracted with EtOAc (40 mL×2). The combined organic layers were washed with H₂O (30 mL×2) and brine (30 mL), dried over anhydrous Na₂SO₄, filtered, and the filtrate was concentrated in vacuo. The resulting residue was purified by column chromatography to give tert-butyl ((S)-5-((4-((1-(tert-butyl)-3-((1S,3R)-3-((tert-butyldimethylsilyl)oxy)cyclopentyl)-1H-pyrazol-5-yl)amino)-6-fluoropyridin-2-yl)oxy)pentan-2-yl)carbamate (130 mg, 73.7% yield) as a yellow oil. LCMS: 634.5 [M+H]⁺.

Step 3: Preparation of tert-butyl (2-(((S)-4-((tert-butoxycarbonyl)amino)pentyl)oxy)-6-fluoropyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-5-((4-((1-(tert-butyl)-3-((1S,3R)-3-((tert-butyldimethylsilyl)oxy)cyclopentyl)-1H-pyrazol-5-yl)amino)-6-fluoropyridin-2-yl)oxy)pentan-2-yl)carbamate (100 mg, 0.16 mmol, 1 eq) and Boc₂O (51.64 mg, 0.24 mmol, 1.5 eq) in DCM (3 mL) was added DMAP (9.64 mg, 0.079 mmol, 0.5 eq), and the mixture was stirred at 25° C. for 3 hours under N₂ atmosphere. After the completion of the reaction, the mixture was then poured into H₂O (40 mL) and extracted with EtOAc (20 mL×2). The combined organic layers were washed with H₂O (30 mL×2) and brine (30 mL), dried over anhydrous Na₂SO₄, filtered, and the filtrate was concentrated in vacuo. The resulting residue was purified by column chromatography to give tert-butyl (2-(((S)-4-((tert-butoxycarbonyl)amino)pentyl)oxy)-6-fluoropyridin-4-yl)(1-(tert-butyl)-3-((1S,3R)-3-((tert-butyldimethylsilyl)oxy)cyclopentyl)-1H-pyrazol-5-yl)carbamate (95 mg, 82.0% yield) as a yellow oil. LCMS: 734.4 [M+H]⁺.

Step 4: Preparation of tert-butyl (2-(((S)-4-((tert-butoxycarbonyl)amino)pentyl)oxy)-6-fluoropyridin-4-yl)(1-(tert-butyl)-3-((1S,3R)-3-hydroxycyclopentyl)-1H-pyrazol-5-yl)carbamate

A mixture of tert-butyl (2-(((S)-4-((tert-butoxycarbonyl)amino)pentyl)oxy)-6-fluoropyridin-4-yl)(1-(tert-butyl)-3-((1S,3R)-3-((tert-butyldimethylsilyl)oxy)cyclopentyl)-1H-pyrazol-5-yl)carbamate (75 mg, 0.10 mmol, 1 eq) and TBAF (1 M, 0.61 mL, 0.60 mmol, 6 eq) in THF (3 mL) was stirred at 25° C. for 1 hour under N₂ atmosphere. After the completion of the reaction, the reaction mixture was quenched with saturated aqueous NH₄Cl (50 mL) and extracted with EtOAC (20 mL×3). The combined organic layers were washed with brine (50 mL), dried over Na₂SO₄, filtered, and the filtrate was concentrated under reduced pressure to give tert-butyl (2-(((S)-4-((tert-butoxycarbonyl)amino)pentyl)oxy)-6-fluoropyridin-4-yl)(1-(tert-butyl)-3-((1S,3R)-3-hydroxycyclopentyl)-1H-pyrazol-5-yl)carbamate (63 mg, 99.5% yield) as a yellow oil. LCMS: 620.3 [M+H]⁺.

Step 5: Preparation of tert-butyl (2-(((S)-4-((tert-butoxycarbonyl)amino)pentyl)oxy)-6-fluoropyridin-4-yl)(1-(tert-butyl)-3-((1S,3R)-3-(((4-nitrophenoxy)carbonyl)oxy)cyclopentyl)-1H-pyrazol-5-yl)carbamate

A mixture of tert-butyl (2-(((S)-4-((tert-butoxycarbonyl)amino)pentyl)oxy)-6-fluoropyridin-4-yl)(1-(tert-butyl)-3-((1S,3R)-3-hydroxycyclopentyl)-1H-pyrazol-5-yl)carbamate (63 mg, 0.10 mmol, 1 eq), (4-nitrophenyl) carbonochloridate (30.73 mg, 0.15 mmol, 1.5 eq), DMAP (6.21 mg, 0.05 mmol, 0.5 eq) and Pyridine (48.24 mg, 0.61 mmol, 6 eq) in DCM (5 mL) was stirred at 25° C. for 3 hours under N₂ atmosphere. After the completion of the reaction, the mixture was poured into H₂O (40 mL) and extracted with EtOAc (40 mL×2). The combined organic layers were washed with H₂O (30 mL×2) and brine (30 mL), dried over anhydrous Na₂SO₄, filtered, and the filtrate was concentrated in vacuo to give tert-butyl (2-(((S)-4-((tert-butoxycarbonyl)amino)pentyl)oxy)-6-fluoropyridin-4-yl)(1-(tert-butyl)-3-((1S,3R)-3-(((4-nitrophenoxy)carbonyl)oxy)cyclopentyl)-1H-pyrazol-5-yl)carbamate (102 mg, 0.13 mmol) as a yellow oil, which was used into the next step without further purification. LCMS: 785.1 [M+H]⁺.

Step 6: Preparation of (1R,3S)-3-(5-((2-(((S)-4-aminopentyl)oxy)-6-fluoropyridin-4-yl)amino)-1-(tert-butyl)-1H-pyrazol-3-yl)cyclopentyl (4-nitrophenyl) carbonate

A mixture of tert-butyl (2-(((S)-4-((tert-butoxycarbonyl)amino)pentyl)oxy)-6-fluoropyridin-4-yl)(1-(tert-butyl)-3-((1S,3R)-3-(((4-nitrophenoxy)carbonyl)oxy)cyclopentyl)-1H-pyrazol-5-yl)carbamate (102 mg, 0.13 mmol, 1 eq) and TFA (74.09 mg, 0.65 mmol, 5 eq) in DCM (3 mL) was stirred at 25° C. for 3 hours under N₂ atmosphere. After the completion of the reaction, the mixture was concentrated in vacuo to give (1R,3S)-3-(5-((2-(((S)-4-aminopentyl)oxy)-6-fluoropyridin-4-yl)amino)-1-(tert-butyl)-1H-pyrazol-3-yl)cyclopentyl (4-nitrophenyl) carbonate (75 mg, 98.7% yield) as a yellow oil, which was used in the next step without further purification. LCMS: 585.3 [M+H]⁺.

Step 7: Preparation of (1¹S,1³R,9S,Z)-2¹-(tert-butyl)-4⁶-fluoro-9-methyl-2¹H-5,12-dioxa-3,10-diaza-4(4,2)-pyridina-2(3,5)-pyrazola-1(1,3)-cyclopentanacyclododecaphan-11-one

A mixture of (1R,3S)-3-(5-((2-(((S)-4-aminopentyl)oxy)-6-fluoropyridin-4-yl)amino)-1-(tert-butyl)-1H-pyrazol-3-yl)cyclopentyl (4-nitrophenyl) carbonate (55 mg, 0.094 mmol, 1 eq) and DIEA (12.16 mg, 0.094 mmol, 1 eq) in THF (55 mL) was stirred at 25° C. for 3 hours under N₂ atmosphere. After the completion of the reaction, the mixture was poured into H₂O (40 mL) and extracted with EtOAc (20 mL×2). The combined organic layers were washed with H₂O (30 mL×2) and brine (30 mL), dried over anhydrous Na₂SO₄, filtered, and the filtrate was concentrated in vacuo to give (1¹S,1³R,9S,Z)-2¹-(tert-butyl)-4⁶-fluoro-9-methyl-2¹H-5,12-dioxa-3,10-diaza-4(4,2)-pyridina-2(3,5)-pyrazola-1(1,3)-cyclopentanacyclododecaphan-11-one (41 mg, 0.092 mmol, 97.8% yield) as a yellow oil, which was used in the next step without further purification. LCMS: 446.2 [M+H]⁺.

Step 11: Preparation of (1¹S,1³R,9S,Z)-4⁶-fluoro-9-methyl-2¹H-5,12-dioxa-3,10-diaza-4(4,2)-pyridina-2(3,5)-pyrazola-1(1,3)-cyclopentanacyclododecaphan-11-one

A mixture of (1¹S,1³R,9S,Z)-2¹-(tert-butyl)-4⁶-fluoro-9-methyl-2¹H-5,12-dioxa-3,10-diaza-4(4,2)-pyridina-2(3,5)-pyrazola-1(1,3)-cyclopentanacyclododecaphan-11-one (36 mg, 0.08 mmol, 1 eq) in HCOOH (6 mL) was stirred at 100° C. for 3 hours under N₂ atmosphere. After the completion of the reaction, the mixture was cooled to room temperature. The mixture was concentrated in vacuo. The resulting residue was purified by prep-HPLC {column: Phenomenex luna C18 150*25 mm*10 um; mobile phase: [water (TFA)-ACN]; gradient:28%-58% B over 10 min} to give (1¹S,1³R,9S,Z)-4⁶-fluoro-9-methyl-2¹H-5,12-dioxa-3,10-diaza-4(4,2)-pyridina-2(3,5)-pyrazola-1(1,3)-cyclopentanacyclododecaphan-11-one (1.94 mg, 6.0% yield) as a white solid. LCMS: 390.2 [M+H]⁺; ¹H NMR (400 MHz, DMSO-d₆) 5=9.24 (s, 1H), 6.97 (d, J=8.0 Hz, 1H), 6.25 (s, 1H), 6.02 (s, 2H), 5.08-5.03 (m, 1H), 4.14-4.00 (m, 2H), 3.59 (d, J=6.4 Hz, 1H), 3.23-3.17 (m, 1H), 2.48-2.37 (m, 1H), 1.82-1.74 (m, 7H), 1.68-1.55 (m, 1H), 1.50-1.46 (m, 6H), 1.07-1.02 (m, 3H) ppm.

Example 38: Synthesis of (1¹S,1³R,9S,Z)-8,8-difluoro-9-methyl-2¹H-5,12-dioxa-3,10-diaza-4(4,2)-pyridina-2(3,5)-pyrazola-1(1,3)-cyclopentanacyclododecaphan-11-one (compound 181)

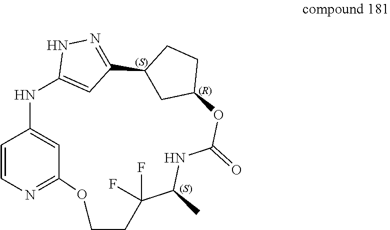

Step 1: Preparation of methyl (S)-4-((tert-butoxycarbonyl)amino)-3-oxopentanoate

To a solution of (tert-butoxycarbonyl)-L-alanine (8 g, 42.28 mmol, 1 eq) in THF (200 mL) was added CDI (8.23 g, 50.74 mmol, 1.2 eq) and the mixture was stirred at 25° C. for 2 hours under N₂ atmosphere, followed by the addition of MgCl2 (4.03 g, 42.28 mmol, 1.74 mL, 1 eq) and potassium 3-methoxy-3-oxo-propanoate (9.91 g, 63.42 mmol, 1.5 eq) in portions. After the completion of the addition, the mixture was stirred at 25° C. for 22 hours under N₂ atmosphere. The mixture was then quenched with aqueous NaHSO₄ (1M, 100 mL) and extracted with Ethyl acetate (100 mL×2). The combined organic layers were washed with brine (100 mL), saturated aqueous NaHCO₃(100 mL) and brine (100 mL), dried over anhydrous Na₂SO₄, filtered, and the filtrate was concentrated. The resulting residue was purified by column chromatography to afford methyl (S)-4-((tert-butoxycarbonyl)amino)-3-oxopentanoate (11.6 g, 89.5% yield) as a white solid. ¹H NMR (400 MHz, CDCl₃) 5=5.14 (s, 1H), 4.45-4.39 (m, 1H), 3.76 (s, 3H), 3.64-3.58 (m, 2H), 1.46 (s, 9H), 1.37 (d, J=7.2 Hz, 3H) ppm.

Step 2: Preparation of methyl (S)-4-((tert-butoxycarbonyl)amino)-3,3-difluoropentanoate

To a solution of methyl (S)-4-((tert-butoxycarbonyl)amino)-3-oxopentanoate (5.68 g, 23.16 mmol, 1 eq) in DCM (60 mL) was added DAST (9.76 g, 60.55 mmol, 8 mL, 2.61 eq) dropwise at 0° C., and the mixture was warmed to 20° C. and kept stirring for 20 hours under N₂ atmosphere. The mixture was then quenched with ice-water (100 mL) and extracted with DCM (100 mL×2). The combined organic layers were washed with saturated aqueous NaHCO₃(100 mL) and brine (30 mL), dried over anhydrous Na₂SO₄, filtered and the filtrate was concentrated. The resulting residue was purified by column chromatography to afford methyl (S)-4-((tert-butoxycarbonyl)amino)-3,3-difluoropentanoate (1.5 g, 24.2% yield) as a yellow oil. 1H NMR (400 MHz, CDCl₃) δ 4.63 (s, 1H), 4.33-4.26 (m, 1H), 3.76 (s, 3H), 3.04-2.96 (m, 2H), 1.47 (s, 9H), 1.28 (d, J=6.4 Hz, 3H) ppm.

Step 3: Preparation of tert-butyl (S)-(3,3-difluoro-5-hydroxypentan-2-yl)carbamate

To the solution of methyl (S)-4-((tert-butoxycarbonyl)amino)-3,3-difluoropentanoate (630 mg, 2.36 mmol, 1 eq) in THF (20 mL) was added LiAlH₄ (2.5 M, 2.36 mL, 2.5 eq) at 0° C., and the mixture was warmed to 25° C. and kept stirring for 2 hours under N₂ atmosphere. After the completion of the reaction, the mixture was sequentially and carefully quenched with H₂O (0.24 mL), 15% NaOH (0.24 mL) and H₂O (0.48 mL). The mixture was filtered, rinsed with ethyl acetate (30 mL), and the filtrate was concentrated to afford tert-butyl (S)-(3,3-difluoro-5-hydroxypentan-2-yl)carbamate (450 mg, 79.8% yield) as a brown oil, which was used into the next step without further purification. ¹H NMR (400 MHz, CDCl₃) δ 4.72-4.69 (m, 1H), 3.89-3.86 (m, 2H), 2.53-2.47 (m, 1H), 2.05 (m, 1H), 1.88-1.81 (m, 2H), 1.45 (s, 9H), 1.26 (d, J=6.4 Hz, 3H) ppm.

Step 4: Preparation of tert-butyl (S)-(5-((4-bromopyridin-2-yl)oxy)-3,3-difluoropentan-2-yl)carbamate

To the solution of tert-butyl (S)-(3,3-difluoro-5-hydroxypentan-2-yl)carbamate (450 mg, 1.88 mmol, 1 eq) and 4-bromo-2-fluoro-pyridine (397.20 mg, 2.26 mmol, 1.2 eq) in THF (10 mL) was added t-BuOK (316.57 mg, 2.82 mmol, 1.5 eq) at 0° C., and the mixture was warmed to 25° C. and kept stirring for 4 hours under N₂ atmosphere. After the reaction was completed, the reaction mixture was quenched with saturated aqueous NH₄Cl (20 mL), diluted with H₂O (20 mL), and extracted with Ethyl acetate (20 mL×3).

The combined organic layers were washed with brine (15 mL×2), dried over Na₂SO₄, filtered, and the filtrate was concentrated under reduced pressure. The resulting residue was purified by column chromatography to afford tert-butyl (S)-(5-((4-bromopyridin-2-yl)oxy)-3,3-difluoropentan-2-yl)carbamate (423 mg, 56.9% yield) as a colorless oil. LCMS: 397.0 [M+H]⁺.

Step 5: Preparation of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-3,3-difluoropentan-2-yl)carbamate

A mixture of tert-butyl (S)-(5-((4-bromopyridin-2-yl)oxy)-3,3-difluoropentan-2-yl)carbamate (400 mg, 1.01 mmol, 1 eq), 1-(tert-butyl)-3-((1S,3R)-3-((tert-butyldimethylsilyl)oxy)cyclopentyl)-1H-pyrazol-5-amine (341.64 mg, 1.01 mmol, 1 eq), Cs₂CO₃ (659.49 mg, 2.02 mmol, 2 eq), Xantphos (117.12 mg, 0.20.41 mmol, 0.2 eq) and Pd₂(dba)₃ (185.35 mg, 0.20 mmol, 0.2 eq) in dioxane (8 mL) was degassed and backfilled with nitrogen atmosphere for 3 times, and stirred at 90° C. for 16 hours under N₂ atmosphere. After the reaction was completed, the mixture was cooled to room temperature. The mixture was filtered through a pad of the Celite, rinsed with dioxane (10 mL), and the filtrate was concentrated in vacuo. The resulting residue was purified by column chromatography to afford tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-3,3-difluoropentan-2-yl)carbamate (380 mg, 57.6% yield) as a brown solid. LCMS: 652.4 [M+H]⁺.

Step 6: Preparation of tert-butyl (2-(((S)-4-((tert-butoxycarbonyl)amino)-3,3-difluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-3,3-difluoropentan-2-yl)carbamate (360 mg, 0.55 mmol, 1 eq) in DCM (10 mL) was added Boc₂O (241.05 mg, 1.10 mmol, 2 eq) and DMAP (67.46 mg, 0.55 mmol, 1 eq), and the mixture was stirred at 25° C. for 12 hours under N₂ atmosphere. The mixture was then concentrated in vacuo. The resulting residue was purified by column chromatography to afford tert-butyl (2-(((S)-4-((tert-butoxycarbonyl)amino)-3,3-difluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate (330 mg, 79.5% yield) as a yellow solid. LCMS: 752.9 [M+H]⁺.

Step 7: Preparation of tert-butyl (2-(((S)-4-((tert-butoxycarbonyl)amino)-3,3-difluoropentyl)oxy)pyridin-4-yl)(1-(tert-butyl)-3-((1S,3R)-3-hydroxycyclopentyl)-1H-pyrazol-5-yl)carbamate

To a solution of tert-butyl (2-(((S)-4-((tert-butoxycarbonyl)amino)-3,3-difluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate (300 mg, 0.40 mmol, 1 eq) in THF (3 mL) was added TBAF (1 M, 2.39 mL, 6 eq), and the mixture was stirred at 25° C. for 4 hours under N₂ atmosphere. The mixture was then quenched with H₂O (50 mL) and extracted with ethyl acetate (50 mL×2). The combined organic layers were washed with brine (50 mL×2), dried over anhydrous Na₂SO₄, filtered, and the filtrate was concentrated to give tert-butyl (2-(((S)-4-((tert-butoxycarbonyl)amino)-3,3-difluoropentyl)oxy)pyridin-4-yl)(1-(tert-butyl)-3-((1S,3R)-3-hydroxycyclopentyl)-1H-pyrazol-5-yl)carbamate (845 mg, crude) as a yellow oil, which was used into the next step without further purification. LCMS: 638.4 [M+H]⁺.

Step 8: Preparation of tert-butyl (2-(((S)-4-((tert-butoxycarbonyl)amino)-3,3-difluoropentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To a solution of tert-butyl (2-(((S)-4-((tert-butoxycarbonyl)amino)-3,3-difluoropentyl)oxy)pyridin-4-yl)(1-(tert-butyl)-3-((1S,3R)-3-hydroxycyclopentyl)-1H-pyrazol-5-yl)carbamate (200 mg, 0.32 mmol, 1 eq) and (4-nitrophenyl) carbonochloridate (126.42 mg, 0.63 mmol, 2 eq) in DCM (8 mL) was added DMAP (38.31 mg, 0.32 mmol, 1 eq) and Pyridine (148.83 mg, 1.88 mmol, 0.15 ml, 6 eq), and the mixture was stirred at 25° C. for 4 hours under N₂ atmosphere. After the completion of the reaction, the mixture was filtered, and the filtrate was concentrated. The resulting residue was purified by column chromatography to afford tert-butyl (2-(((S)-4-((tert-butoxycarbonyl)amino)-3,3-difluoropentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (220 mg, 0.27 mmol, 87.4% yield) as a white solid. LCMS: 803.3 [M+H]⁺.

Step 9: Preparation of (1R,3S)-3-(5-((2-(((S)-4-amino-3,3-difluoropentyl)oxy)pyridin-4-yl)amino)-1-(tert-butyl)-1H-pyrazol-3-yl)cyclopentyl (4-nitrophenyl) carbonate

To a solution of tert-butyl (2-(((S)-4-((tert-butoxycarbonyl)amino)-3,3-difluoropentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (200 mg, 0.25 mmol, 1 eq) in DCM (3 mL) was added TFA (170 mg, 1.49 mmol, 0.11 ml, 6 eq), and the mixture was stirred at 25° C. for 4 hours under N₂ atmosphere. After the completion of the reaction, the mixture was concentrated to afford (1R,3S)-3-(5-((2-(((S)-4-amino-3,3-difluoropentyl)oxy)pyridin-4-yl)amino)-1-(tert-butyl)-1H-pyrazol-3-yl)cyclopentyl (4-nitrophenyl) carbonate (150 mg, 0.25 mmol) as a brown oil, which was used into the next step without further purification. LCMS: 603.2 [M+H]⁺.

Step 10: Preparation of (1¹S,1³R,9S,Z)-2¹-(tert-butyl)-8,8-difluoro-9-methyl-2¹H-5,12-dioxa-3,10-diaza-4(4,2)-pyridina-2(3,5)-pyrazola-1(1,3)-cyclopentanacyclododecaphan-11-one

To a solution of (1R,3S)-3-(5-((2-(((S)-4-amino-3,3-difluoropentyl)oxy)pyridin-4-yl)amino)-1-(tert-butyl)-1H-pyrazol-3-yl)cyclopentyl (4-nitrophenyl) carbonate (140 mg, 0.23 mmol, 1 eq) in DMSO (200 mL) was added DIEA (180.15 mg, 1.39 mmol, 6 eq), and the mixture was stirred at 100° C. for 16 hours under N₂ atmosphere. The mixture was then cooled to room temperature and concentrated under reduced pressure to afford (1¹S,1³R,9S,Z)-2¹-(tert-butyl)-8,8-difluoro-9-methyl-2¹H-5,12-dioxa-3,10-diaza-4(4,2)-pyridina-2(3,5)-pyrazola-1 (1,3)-cyclopentanacyclododecaphan-11-one (94 mg, 0.20 mmol) as a brown oil, which was used into the next step without further purification. LCMS: 464.3 [M+H]⁺.

Step 11: Preparation of (1¹S,1³R,9S,Z)-8,8-difluoro-9-methyl-2¹H-5,12-dioxa-3,10-diaza-4(4,2)-pyridina-2(3,5)-pyrazola-1(1,3)-cyclopentanacyclododecaphan-11-one

A solution of (1¹S,1³R,9S,Z)-2¹-(tert-butyl)-8,8-difluoro-9-methyl-2¹H-5,12-dioxa-3,10-diaza-4(4,2)-pyridina-2(3,5)-pyrazola-1(1,3)-cyclopentanacyclododecaphan-11-one (80 mg, 0.17 mmol, 1 eq) in formic acid (1 mL) was stirred at 100° C. for 2 hours under N₂ atmosphere. After the completion of the reaction, the mixture was cooled to room temperature and the volatiles were removed under reduced pressure. The resulting residue was purified by prep-HPLC (column: Phenomenex luna C18 150*25 mm*10 um; mobile phase: [water (FA)-ACN]; gradient:5%-35% B over 10 min) and further by SFC (column: DAICEL CHIRALPAK AS (250 mm*30 mm, 10 um); mobile phase: [CO₂-i-PrOH (0.1% NH₃·H₂O)]; B %:35%, isocratic elution mode) to afford compound (1¹S,1³R,9S,Z)-8,8-difluoro-9-methyl-2¹H-5,12-dioxa-3,10-diaza-4(4,2)-pyridina-2(3,5)-pyrazola-1(1,3)-cyclopentanacyclododecaphan-11-one (23.67 mg, 33.2% yield, 98.6% purity) as a white solid. LCMS: 408.3 [M+H]⁺; ¹H NMR (400 MHz, DMSO-d₆) δ 12.12-12.07 (m, 1H), 9.00-8.94 (m, 1H), 7.76 (d, J=6.0 Hz, 1H), 7.45-7.40 (m, 1H), 6.44 (dd, J=1.2, 5.6 Hz, 1H), 6.34-6.32 (m, 1H), 6.10-6.03 (m, 1H), 5.11-5.10 (m, 1H), 4.27-4.22 (m, 2H), 4.07-3.90 (m, 1H), 3.23-3.19 (m, 1H), 2.30-2.18 (m, 1H), 2.14-1.92 (m, 2H), 1.80-1.70 (m, 4H), 1.28-1.20 (m, 1H), 1.18-1.08 (m, 3H) ppm.

Example 39: synthesis of (1¹S,1³R,9S,Z)-8,8-difluoro-9-methyl-2¹H-5,12-dioxa-3,10-diaza-4(4,2)-pyridina-2(3,5)-pyrazola-1(1,3)-cyclopentanacyclododecaphan-11-one-6,6-d₂ (compound 182)

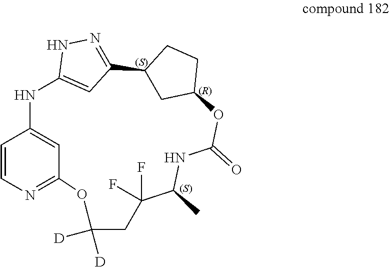

Step 1: Preparation of tert-butyl (S)-(3,3-difluoro-5-hydroxypentan-2-yl-5,5-d₂)carbamate

To the solution of methyl (S)-4-((tert-butoxycarbonyl)amino)-3,3-difluoropentanoate (700 mg, 2.62 mmol, 1 eq) in THF (10 mL) at 0° C. was added LiAlD₄ (350 mg, 7.61 mmol, 2.90 eq), and the mixture was warmed to 25° C. and kept stirring at that temperature for 1 hours under N₂ atmosphere. After the completion of the reaction, the mixture reaction was sequentially and carefully quenched with H₂O (0.35 mL), 15% NaOH (0.35 mL) and H₂O (0.7 mL). The mixture was filtered, rinsed with THF (10 mL) and the filtrate was concentrated in vacuo to afford tert-butyl (S)-(3,3-difluoro-5-hydroxypentan-2-yl-5,5-d₂)carbamate (466 mg, 1.93 mmol, 73.7% yield) as a yellow oil.

Step 2: Preparation of tert-butyl (S)-(5-((4-bromopyridin-2-yl)oxy)-3,3-difluoropentan-2-yl-5,5-d₂)carbamate

To the solution of 4-bromo-2-fluoro-pyridine (300 mg, 1.70 mmol, 1 eq) and tert-butyl (S)-(3,3-difluoro-5-hydroxypentan-2-yl-5,5-d₂)carbamate (466 mg, 1.93 mmol, 1.13 eq) in THF (10 mL) at 0° C. was added t-BuOK (290 mg, 2.58 mmol, 1.52 eq), and the mixture was warmed to 25° C. and stirred for 12 hours under N₂ atmosphere. The mixture was quenched with saturated aqueous NH₄Cl (50 mL) and extracted with EtOAc (50 mL×2). The combined organic layers were washed with saturated brine (50 mL), dried over anhydrous Na₂SO₄, filtered, and the filtrate was concentrated in vacuo. The resulting residue was purified by column chromatography to afford tert-butyl (S)-(5-((4-bromopyridin-2-yl)oxy)-3,3-difluoropentan-2-yl-5,5-d₂)carbamate (435 mg, 62.9% yield) as a colorless oil. LCMS: 397.0 [M+H]⁺, 341.0 [M−55+H]⁺.

Step 3: Preparation of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-3,3-difluoropentan-2-yl-5,5-d₂)carbamate

The mixture of tert-butyl (S)-(5-((4-bromopyridin-2-yl)oxy)-3,3-difluoropentan-2-yl-5,5-d₂)carbamate (400 mg, 1.01 mmol, 1.03 eq), 1-(tert-butyl)-3-((1S,3R)-3-((tert-butyldimethylsilyl)oxy)cyclopentyl)-1H-pyrazol-5-amine (330 mg, 0.978 mmol, 1 eq), Cs₂CO₃ (955.53 mg, 2.93 mmol, 3 eq), Xantphos (113.13 mg, 0.196 mmol, 0.2 eq) and Pd₂(dba)₃ (89.52 mg, 0.098 mmol, 0.1 eq) in dioxane (10 mL) was degassed and back-filled with N₂ for three times, and the mixture was stirred at 90° C. for 12 hours under N₂ atmosphere. After the completion of the reaction, the mixture was cooled to room temperature. The mixture was filtered through a pad of the Celite, rinsed with dioxane (10 mL) and the filtrate was concentrated in vacuo. The resulting residue was purified by column chromatography to afford tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-3,3-difluoropentan-2-yl-5,5-d₂)carbamate (635 mg, 96.4% yield) as a yellow oil. LCMS: 654.4 [M+H]⁺.

Step 4: Preparation of tert-butyl (2-(((S)-4-((tert-butoxycarbonyl)amino)-3,3-difluoropentyl-1,1-d₂)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-3,3-difluoropentan-2-yl-5,5-d₂)carbamate (635 mg, 0.971 mmol, 1 eq) in DCM (10 mL) were added DMAP (119 mg, 0.974 mmol, 1 eq) and Boc₂O (318 mg, 1.46 mmol, 334.74 μL, 1.5 eq), and the reaction mixture was stirred at 25° C. for 3 hours under N₂ atmosphere. After the completion of the reaction, the mixture was concentrated in vacuo. The resulting residue was purified by column chromatography to afford tert-butyl (2-(((S)-4-((tert-butoxycarbonyl)amino)-3,3-difluoropentyl-1,1-d₂)oxy)pyridin-4-yl)(1-(tert-butyl)-3-((1S,3R)-3-((tert-butyldimethylsilyl)oxy)cyclopentyl)-1H-pyrazol-5-yl)carbamate (860 mg, 99.6% yield) as a yellow oil. LCMS: 754.5 [M+H]⁺.

Step 5: Preparation of tert-butyl (2-(((S)-4-((tert-butoxycarbonyl)amino)-3,3-difluoropentyl-1,1-d₂)oxy)pyridin-4-yl)(1-(tert-butyl)-3-((1S,3R)-3-hydroxycyclopentyl)-1H-pyrazol-5-yl)carbamate

To the a solution of tert-butyl (2-(((S)-4-((tert-butoxycarbonyl)amino)-3,3-difluoropentyl-1,1-d₂)oxy)pyridin-4-yl)(1-(tert-butyl)-3-((1S,3R)-3-((tert-butyldimethylsilyl)oxy)cyclopentyl)-1H-pyrazol-5-yl)carbamate (860 mg, 1.14 mmol, 1 eq) in THF (10 mL) was added TBAF (1 M, 6 mL, 5.26 eq), and the reaction mixture was stirred at 25° C. for 12 hours under N₂ atmosphere. After the completion of the reaction, the mixture was quenched with H₂O (50 mL) and extracted with EtOAc (50 mL×2). The combined organic layers were washed with saturated brine (50 mL×2), dried over anhydrous Na₂SO₄, filtered and the filtrate was concentrated in vacuo. The desired tert-butyl (2-(((S)-4-((tert-butoxycarbonyl)amino)-3,3-difluoropentyl-1,1-d₂)oxy)pyridin-4-yl)(1-(tert-butyl)-3-((1S,3R)-3-hydroxycyclopentyl)-1H-pyrazol-5-yl)carbamate (845 mg, 1.32 mmol, crude product) was obtained as a yellow oil, which was used into the next step without further purification. LCMS: 640.2 [M+H]⁺.

Step 6: Preparation of tert-butyl (2-(((S)-4-((tert-butoxycarbonyl)amino)-3,3-difluoropentyl-1,1-d₂)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

To the solution of tert-butyl (2-(((S)-4-((tert-butoxycarbonyl)amino)-3,3-difluoropentyl-1,1-d₂)oxy)pyridin-4-yl)(1-(tert-butyl)-3-((1S,3R)-3-hydroxycyclopentyl)-1H-pyrazol-5-yl)carbamate (545 mg, 0.852 mmol, 1 eq), (4-nitrophenyl) carbonochloridate (516 mg, 2.56 mmol, 3.0 eq) and DMAP (52.04 mg, 0.426 mmol, 0.5 eq) in THF (10 mL) was added pyridine (441.00 mg, 5.58 mmol, 0.45 mL, 6.54 eq) dropwise, and the reaction mixture was stirred at 25° C. for 0.5 hour under N₂ atmosphere. After the completion of the reaction, the mixture was filtered, rinsed with THF (10 mL) and the filtrate was concentrated in vacuo. The resulting residue was purified by column chromatography to afford tert-butyl (2-(((S)-4-((tert-butoxycarbonyl)amino)-3,3-difluoropentyl-1,1-d₂)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (530 mg, 77.3% yield) as a colorless oil. LCMS: 805.1 [M+H]⁺.

Step 7: Preparation of (1R,3S)-3-(5-((2-(((S)-4-amino-3,3-difluoropentyl-1,1-d₂)oxy)pyridin-4-yl)amino)-1-(tert-butyl)-1H-pyrazol-3-yl)cyclopentyl (4-nitrophenyl) carbonate

To the solution of tert-butyl (2-(((S)-4-((tert-butoxycarbonyl)amino)-3,3-difluoropentyl-1,1-d₂)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (480 mg, 0.596 mmol, 1 eq) in DCM (10 mL) was added TFA (2 mL) and the mixture was stirred at 25° C. for 12 hours under N₂ atmosphere. The mixture was then concentrated in vacuo to afford (1R,3S)-3-(5-((2-(((S)-4-amino-3,3-difluoropentyl-1,1-d₂)oxy)pyridin-4-yl)amino)-1-(tert-butyl)-1H-pyrazol-3-yl)cyclopentyl (4-nitrophenyl) carbonate (500 mg, crude) as a colorless oil, which was used into the next step without further purification. LCMS: 605.3 [M+H]⁺.

Step 8: Preparation of (1¹S,1³R,9S,Z)-2¹-(tert-butyl)-8,8-difluoro-9-methyl-2¹H-5,12-dioxa-3,10-diaza-4(4,2)-pyridina-2(3,5)-pyrazola-1(1,3)-cyclopentanacyclododecaphan-11-one-6,6-d₂

To the solution of (1R,3S)-3-(5-((2-(((S)-4-amino-3,3-difluoropentyl-1,1-d₂)oxy)pyridin-4-yl)amino)-1-(tert-butyl)-1H-pyrazol-3-yl)cyclopentyl (4-nitrophenyl) carbonate (500 mg, 0.696 mmol, 1 eq) in DMSO (500 mL) was added DIEA (1.48 g, 11.48 mmol, 2 mL, 16.50 eq) and the mixture was stirred at 100° C. for 4 hours under N₂ atmosphere. After the completion of the reaction, the mixture was cooled to room temperature. The mixture was then poured into H₂O (400 mL) and extracted with EtOAc (200 mL×2). The combined organic layers were washed with H₂O (300 mL×2) and saturated brine (300 mL), dried over anhydrous Na₂SO₄, filtered and the filtrate was concentrated in vacuo.

The desired (1¹S,1³R,9S,Z)-2¹-(tert-butyl)-8,8-difluoro-9-methyl-2¹H-5,12-dioxa-3,10-diaza-4(4,2)-pyridina-2(3,5)-pyrazola-1(1,3)-cyclopentanacyclododecaphan-11-one-6,6-d₂ (370 mg, crude) was obtained as a yellow oil. LCMS: 466.3 [M+H]⁺.

Step 9: Preparation of (1¹S,1³R,9S,Z)-8,8-difluoro-9-methyl-2¹H-5,12-dioxa-3,10-diaza-4(4,2)-pyridina-2(3,5)-pyrazola-1(1,3)-cyclopentanacyclododecaphan-11-one-6,6-d₂

The mixture of (1¹S,1³R,9S,Z)-2¹-(tert-butyl)-8,8-difluoro-9-methyl-2¹H-5,12-dioxa-3,10-diaza-4(4,2)-pyridina-2(3,5)-pyrazola-1(1,3)-cyclopentanacyclododecaphan-11-one-6,6-d₂ (370 mg, 0.557 mmol, 1 eq) and formic acid (8 mL) was stirred at 100° C. for 0.5 hour under N₂ atmosphere. After the completion of the reaction, the mixture was cooled to room temperature and the volatiles were removed in vacuo. The resulting residue was purified by prep-HPLC {column: Phenomenex luna C18 150*40 mm*15 um; mobile phase: [water(FA)-ACN]; gradient:7%-37% B over 15 min} to afford compound (1¹S,1³R,9S,Z)-8,8-difluoro-9-methyl-2¹H-5,12-dioxa-3,10-diaza-4(4,2)-pyridina-2(3,5)-pyrazola-1(1,3)-cyclopentanacyclododecaphan-11-one-6,6-d₂ (111.8 mg, 47.9% yield) as a white solid. LCMS: 410.2 [M+H]⁺; ¹H NMR (400 MHz, DMSO-d₆) δ 12.27-12.08 (m, 1H), 8.97 (s, 1H), 8.14 (s, 1H), 7.77 (d, J=4.8 Hz, 1H), 7.43 (br d, J=8.8 Hz, 1H), 6.46 (dd, J=1.6, 5.6 Hz, 1H), 6.33 (s, 1H), 6.04 (s, 1H), 5.12 (brs, 1H), 4.00-3.96 (m, 1H), 3.26-3.21 (m, 1H), 2.47-2.40 (m, 2H), 2.29-2.16 (m, 1H), 2.10-2.06 (m, 1H), 1.81 (s, 3H), 1.75-1.71 (m, 1H), 1.15-1.13 (m, 3H) ppm.

Example 40: synthesis of (1¹S,1³R,9S,E)-2⁴-fluoro-9-methyl-2¹H-5,12-dioxa-3,10-diaza-4(4,2)-pyridina-2(3,5)-pyrazola-1(1,3)-cyclopentanacyclododecaphan-11-one (compound 183)

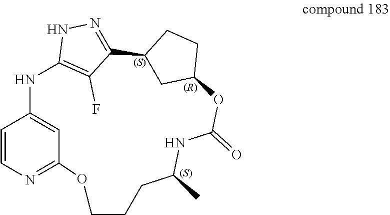

Step 1: Preparation of (11S,13R,9S,E)-21-(tert-butyl)-24-fluoro-9-methyl-21H-5,12-dioxa-3,10-diaza-4(4,2)-pyridina-2(3,5)-pyrazola-1(1,3)-cyclopentanacyclododecaphan-11-one

To a solution of (1¹S,1³R,9S,Z)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-11-one (100 mg, 0.23 mmol, 1 eq) in CH₃CN (2 mL) was added selectfluor (91.15 mg, 0.25 mmol, 1.1 eq), and the mixture was stirred at 25° C. for 12 hours under N₂ atmosphere. After the reaction was completed, the mixture was quenched with saturated aqueous NaHCO₃ (50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by prep-TLC (PE:EA=1:2) to afford (1¹S,1³R,9S,E)-2¹-(tert-butyl)-2⁴-fluoro-9-methyl-2¹H-5,12-dioxa-3,10-diaza-4(4,2)-pyridina-2(3,5)-pyrazola-1(1,3)-cyclopentanacyclododecaphan-11-one (67 mg, 64.3% yield) as a light-yellow oil. LCMS: 446.2 [M+H]⁺; ¹H NMR (400 MHz, DMSO-d₆) δ 8.39 (s, 1H), 7.81 (d, J=5.63 Hz, 1H), 6.61 (br d, J=8.38 Hz, 1H), 6.54 (dd, J=5.63, 1.75 Hz, 1H), 5.50 (br s, 1H), 4.94 (br s, 1H), 4.09-3.94 (m, 1H), 3.93-3.83 (m, 1H), 3.64-3.47 (m, 1H), 3.24-3.10 (m, 1H), 2.46-2.25 (m, 2H), 1.95-1.79 (m, 3H), 1.77-1.58 (m, 3H), 1.49 (s, 9H), 1.28-1.12 (m, 2H), 0.98 (br d, J=6.38 Hz, 3H) ppm.

Step 2: Preparation of (1¹S,1³R,9S,E)-2⁴-fluoro-9-methyl-2¹H-5,12-dioxa-3,10-diaza-4(4,2)-pyridina-2(3,5)-pyrazola-1(1,3)-cyclopentanacyclododecaphan-11-one

A mixture of (1¹S,1³R,9S,E)-2¹-(tert-butyl)-2⁴-fluoro-9-methyl-2¹H-5,12-dioxa-3,10-diaza-4(4,2)-pyridina-2(3,5)-pyrazola-1(1,3)-cyclopentanacyclododecaphan-11-one (59 mg, 0.13 mmol, 1 eq) and formic acid (1.5 mL) was stirred at 100° C. for 1 hours under N₂ atmosphere. The mixture was then cooled to room temperature and concentrated in vacuo. The resulting residue was purified by prep-HPLC (neutral condition:column: Waters Xbridge 150×25 mm×5 um; mobile phase: [water (NH₄HCO₃)-ACN]; gradient:16%-36% B over 10 min) to afford compound (1¹S,1³R,9S,E)-2⁴-fluoro-9-methyl-2¹H-5,12-dioxa-3,10-diaza-4(4,2)-pyridina-2(3,5)-pyrazola-1(1,3)-cyclopentanacyclododecaphan-11-one (26.54 mg, 47.8% yield) as a white solid. LCMS: 390.2 [M+H]⁺; ¹H NMR (400 MHz, DMSO-d₆) δ 12.31-12.23 (m, 1H), 8.72 (s, 1H), 7.75 (d, J=5.6 Hz, 1H), 6.76-6.69 (m, 1H), 6.42 (dd, J=1.7, 5.7 Hz, 1H), 5.81 (br s, 1H), 5.02-4.91 (m, 1H), 3.99-3.88 (m, 2H), 3.65-3.49 (m, 1H), 2.43-2.30 (m, 2H), 2.26-2.11 (m, 1H), 2.04-1.88 (m, 2H), 1.85-1.80 (m, 2H), 1.75-1.61 (m, 2H), 1.54-1.38 (m, 2H), 1.00 (br d, J=6.6 Hz, 3H) ppm.

Example 41: synthesis of (1¹S,1³R,9S,E)-2⁴-chloro-9-methyl-2¹H-5,12-dioxa-3,10-diaza-4(4,2)-pyridina-2(3,5)-pyrazola-1(1,3)-cyclopentanacyclododecaphan-11-one (compound 184)

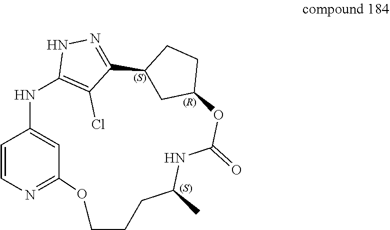

Step 1: Preparation of (11S,13R,9S,E)-21-(tert-butyl)-24-chloro-9-methyl-21H-5,12-dioxa-3,10-diaza-4(4,2)-pyridina-2(3,5)-pyrazola-1(1,3)-cyclopentanacyclododecaphan-11-one

To a solution of (1¹S,1³R,9S,Z)-2¹-(tert-butyl)-9-methyl-2¹H-5,12-dioxa-3,10-diaza-4(4,2)-pyridina-2(3,5)-pyrazola-1(1,3)-cyclopentanacyclododecaphan-11-one (100 mg, 0.23 mmol, 1 eq) in CH₃CN (2 mL) was added NCS (31.23 mg, 0.23 mmol, 1 eq), and the mixture was stirred at 25° C. for 12 hours under N₂ atmosphere. The mixture was then quenched with H₂O (50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by prep-TLC (PE:EA=1:2) to afford (1¹S,1³R,9S,E)-2¹-(tert-butyl)-2⁴-chloro-9-methyl-2¹H-5,12-dioxa-3,10-diaza-4(4,2)-pyridina-2(3,5)-pyrazola-1(1,3)-cyclopentanacyclododecaphan-11-one (50 mg, 0.10 mmol, 46.3% yield) as a yellow oil. LCMS: 462.2 [M+H]⁺.

Step 2: Preparation of (1¹S,1³R,9S,E)-2⁴-chloro-9-methyl-2¹H-5,12-dioxa-3,10-diaza-4(4,2)-pyridina-2(3,5)-pyrazola-1(1,3)-cyclopentanacyclododecaphan-11-one

A mixture of (1¹S,1³R,9S,E)-2¹-(tert-butyl)-2⁴-chloro-9-methyl-2¹H-5,12-dioxa-3,10-diaza-4(4,2)-pyridina-2(3,5)-pyrazola-1(1,3)-cyclopentanacyclododecaphan-11-one (45 mg, 0.097 mmol, 1 eq) and formic acid (1.3 mL) was stirred at 100° C. for 1 hour under N₂ atmosphere. The mixture was then cooled to room temperature and concentrated in vacuo. The resulting residue was purified by prep-HPLC (FA condition: column: Phenomenex luna C18 150×25 mm×10 um; mobile phase: [water (FA)-MeOH]; gradient:10%-37% B over 9 min) to afford (1¹S,1³R,9S,E)-2⁴-chloro-9-methyl-2¹H-5,12-dioxa-3,10-diaza-4(4,2)-pyridina-2(3,5)-pyrazola-1(1,3)-cyclopentanacyclododecaphan-11-one (23.29 mg, 58.5% yield) as a white solid. LCMS: 406.2 [M+H]⁺; ¹H NMR (400 MHz, DMSO-d₆) δ 12.82-12.43 (m, 1H), 8.70-8.63 (m, 1H), 8.13 (s, 1H), 7.78-7.71 (m, 1H), 6.74-6.67 (m, 1H), 6.46-6.39 (m, 1H), 5.78-5.63 (m, 1H), 5.03-4.90 (m, 1H), 3.91-3.82 (m, 2H), 3.56-3.51 (m, 1H), 3.20-3.13 (m, 1H), 2.47-2.26 (m, 2H), 1.91 (br s, 4H), 1.77-1.59 (m, 2H), 1.58-1.45 (m, 1H), 1.44-1.31 (m, 1H), 0.99 (br d, J=6.5 Hz, 3H) ppm.

Example 42: synthesis of (1¹S,1³R,9S,E)-2⁴,8,8-trifluoro-9-methyl-2¹H-5,12-dioxa-3,10-diaza-4(4,2)-pyridina-2(3,5)-pyrazola-1 (1,3)-cyclopentanacyclododecaphan-11-one (compound 188)

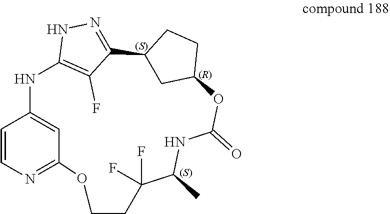

Step 1: Preparation of (1¹S,1³R,9S,E)-2¹-(tert-butyl)-24,8,8-trifluoro-9-methyl-2¹H-5,12-dioxa-3,10-diaza-4(4,2)-pyridina-2(3,5)-pyrazola-1(1,3)-cyclopentanacyclododecaphan-11-one

To a solution of (1¹S,1³R,9S,Z)-2¹-(tert-butyl)-8,8-difluoro-9-methyl-2¹H-5,12-dioxa-3,10-diaza-4(4,2)-pyridina-2(3,5)-pyrazola-1(1,3)-cyclopentanacyclododecaphan-11-one (160 mg, 0.35 mmol, 1 eq) in CH₃CN (3 mL) was added selectfluor (134.5 mg, 0.38 mmol, 1.1 eq), and the mixture was stirred at 20° C. for 16 hours under N₂ atmosphere. After the completion of the reaction, the mixture was quenched with saturated aqueous NaHCO₃(50 mL) and extracted with EtOAc (50 mL×2). The combined organic layers were washed with brine (50 mL), dried over anhydrous Na₂SO₄, filtered and the filtrate was concentrated in vacuo. The resulting residue was purified by prep-TLC (PE:EA=1:1) to afford compound (1¹S,1³R,9S,E)-2¹-(tert-butyl)-24,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (110 mg, 0.23 mmol, 66.2% yield) as a white gum. LCMS: 482.3 [M+H]⁺.

Step 2: Preparation of (1¹S,1³R,9S,E)-2⁴,8,8-trifluoro-9-methyl-2¹H-5,12-dioxa-3,10-diaza-4(4,2)-pyridina-2(3,5)-pyrazola-1 (1,3)-cyclopentanacyclododecaphan-11-one

A mixture of (1¹S,1³R,9S,E)-2¹-(tert-butyl)-24,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (100 mg, 0.21 mmol, 1 eq) and formic acid (5 mL) was stirred at 100° C. for 0.5 hour under N₂ atmosphere. After the completion of the reaction, the mixture was cooled to room temperature and concentrated in vacuo. The resulting residue was purified by prep-HPLC (column: Welch Xtimate C18 150*25 mm*5 um; mobile phase: [water(FA)-ACN]; gradient:8%-38% B over 10 min) to afford compound (1¹S,1³R,9S,E)-2⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (42.53 mg, 0.089 mmol, 42.9% yield, 98.7% purity) as a white solid. LCMS: 426.1 [M+H]⁺; ¹H NMR (400 MHz, DMSO-d₆) δ 12.47-12.10 (m, 1H), 8.79 (s, 1H), 8.14 (s, 1H), 7.78 (d, J=5.6 Hz, 1H), 7.43-7.19 (m, 1H), 6.46 (dd, J=5.6, 1.6 Hz, 1H), 5.87 (br d, J=1.2 Hz, 1H), 5.04 (br s, 1H), 4.29-4.01 (m, 2H), 4.00-3.86 (m, 1H), 3.24 (br dd, J=6.4, 3.8 Hz, 1H), 2.41 (dd, J=10.0, 5.0 Hz, 1H), 2.34-2.17 (m, 3H), 2.06-1.97 (m, 1H), 1.95-1.82 (m, 3H), 1.11 (br d, J=7.2 Hz, 3H) ppm.

Example 43: synthesis of (1¹S,1³R,9S,Z)-8,8-difluoro-9-methyl-2¹H-5,12-dioxa-3,10-diaza-4(2,6)-pyrazina-2(3,5)-pyrazola-1(1,3)-cyclopentanacyclododecaphan-11-one (compound 219)

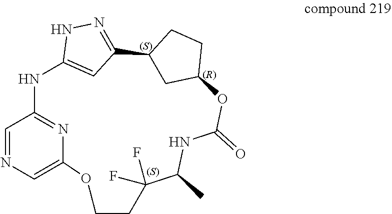

Step 1: Preparation of tert-butyl (S)-(5-((6-bromopyrazin-2-yl)oxy)-3,3-difluoropentan-2-yl)carbamate

A mixture of tert-butyl (S)-(3,3-difluoro-5-hydroxypentan-2-yl)carbamate (210 mg, 0.88 mmol, 1 eq), 2-bromo-6-fluoropyrazine (170 mg, 0.965 mmol, 1.1 eq) and t-BuOK (295.5 mg, 2.63 mmol, 3 eq) in THF (10 mL) was stirred at 25° C. for 1 hour under N₂ atmosphere. After the completion of the reaction, the mixture was poured into H₂O (50 mL) and extracted with EtOAc (50 mL×2). The combined organic layers were washed with brine (30 mL), dried over anhydrous Na₂SO₄, filtered, and the filtrate was concentrated in vacuo. The resulting residue was purified by column chromatography to give compound tert-butyl (S)-(5-((6-bromopyrazin-2-yl)oxy)-3,3-difluoropentan-2-yl)carbamate (90 mg, 25.9% yield) as a yellow oil.

Step 2: Preparation of tert-butyl ((S)-5-((6-((1-(tert-butyl)-3-((1S,3R)-3-((tert-butyldimethylsilyl)oxy)cyclopentyl)-1H-pyrazol-5-yl)amino)pyrazin-2-yl)oxy)-3,3-difluoropentan-2-yl)carbamate

A mixture of tert-butyl (S)-(5-((6-bromopyrazin-2-yl)oxy)-3,3-difluoropentan-2-yl)carbamate (90 mg, 0.227 mmol, 1 eq), 1-(tert-butyl)-3-((1S,3R)-3-((tert-butyldimethylsilyl)oxy)cyclopentyl)-1H-pyrazol-5-amine (84 mg, 0.249 mmol, 1.1 eq). Pd₂(dba)₃ (20.8 mg, 0.023 mmol, 0.1 eq), Xantphos (26.3 mg, 0.045 mmol, 0.2 eq) and Cs₂CO₃ (222 mg, 0.681 mmol, 3 eq) in dioxane (5 mL) was stirred at 90° C. for 12 hours under N₂ atmosphere. After the completion of the reaction, the mixture was cooled to room temperature. The mixture was diluted with H₂O (50 mL) and extracted with EtOAc (50 mL×2). The combined organic layers were washed with brine (30 mL×2), dried over anhydrous Na₂SO₄, filtered, and the filtrate was concentrated in vacuo. The resulting residue was purified by column chromatography to give tert-butyl ((S)-5-((6-((1-(tert-butyl)-3-((1S,3R)-3-((tert-butyldimethylsilyl)oxy)cyclopentyl)-1H-pyrazol-5-yl)amino)pyrazin-2-yl)oxy)-3,3-difluoropentan-2-yl)carbamate (100 mg, 67.5% yield) as a yellow oil. LCMS: 653.5 [M+H]⁺.

Step 3: Preparation of tert-butyl (6-(((S)-4-((tert-butoxycarbonyl)amino)-3,3-difluoropentyl)oxy)pyrazin-2-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-5-((6-((1-(tert-butyl)-3-((1S,3R)-3-((tert-butyldimethylsilyl)oxy)cyclopentyl)-1H-pyrazol-5-yl)amino)pyrazin-2-yl)oxy)-3,3-difluoropentan-2-yl)carbamate (100 mg, 0.153 mmol, 1 eq) and Boc₂O (50 mg, 0.23 mmol, 1.5 eq) in DCM (3 mL) was added DMAP (9.4 mg, 0.077 mmol, 0.5 eq), and the mixture was stirred at 25° C. for 12 hours under N₂ atmosphere. After the completion of the reaction, the mixture was quenched with H₂O (20 mL) and extracted with EtOAc (20 mL×2). The combined organic layers were washed with brine (20 mL), dried over anhydrous Na₂SO₄, filtered, and the filtrate was concentrated in vacuo. The resulting residue was purified by column chromatography to give tert-butyl (6-(((S)-4-((tert-butoxycarbonyl)amino)-3,3-difluoropentyl)oxy)pyrazin-2-yl)(1-(tert-butyl)-3-((1S,3R)-3-((tert-butyldimethylsilyl)oxy)cyclopentyl)-1H-pyrazol-5-yl)carbamate (90 mg, 0.119 mmol, 78.1% yield) as a yellow oil. LCMS: 753.7 [M+H]⁺.

Step 4: Preparation of tert-butyl (6-(((S)-4-((tert-butoxycarbonyl)amino)-3,3-difluoropentyl)oxy)pyrazin-2-yl)(1-(tert-butyl)-3-((1S,3R)-3-hydroxycyclopentyl)-1H-pyrazol-5-yl)carbamate

A mixture of tert-butyl (6-(((S)-4-((tert-butoxycarbonyl)amino)-3,3-difluoropentyl)oxy)pyrazin-2-yl)(1-(tert-butyl)-3-((1S,3R)-3-((tert-butyldimethylsilyl)oxy)cyclopentyl)-1H-pyrazol-5-yl)carbamate (90 mg, 0.119 mmol, 1 eq) and TBAF (1 M, 0.12 mL, 1 eq) in THF (3 mL) was stirred at 25° C. for 1 hour under N₂ atmosphere. The reaction mixture was then quenched with saturated aqueous NH₄Cl (20 mL) and extracted with EtOAc (20 mL×3). The combined organic layers were washed with brine (10 mL), dried over Na₂SO₄, filtered, and the filtrate was concentrated under reduced pressure. The desired tert-butyl (6-(((S)-4-((tert-butoxycarbonyl)amino)-3,3-difluoropentyl)oxy)pyrazin-2-yl)(1-(tert-butyl)-3-((1S,3R)-3-hydroxycyclopentyl)-1H-pyrazol-5-yl)carbamate (126 mg, crude) was obtained as a yellow oil, which was used in the next step without further purification. LCMS: 639.4 [M+H]⁺.

Step 5: Preparation of tert-butyl (6-(((S)-4-((tert-butoxycarbonyl)amino)-3,3-difluoropentyl)oxy)pyrazin-2-yl)(1-(tert-butyl)-3-((1S,3R)-3-(((4-nitrophenoxy)carbonyl)oxy)cyclopentyl)-1H-pyrazol-5-yl)carbamate

The mixture of tert-butyl (6-(((S)-4-((tert-butoxycarbonyl)amino)-3,3-difluoropentyl)oxy)pyrazin-2-yl)(1-(tert-butyl)-3-((1S,3R)-3-hydroxycyclopentyl)-1H-pyrazol-5-yl)carbamate (126 mg, 0.197 mmol, 1 eq), 4-nitrophenyl carbonochloridate (119 mg, 0.591 mmol, 3 eq) and DMAP (12 mg, 0.098 mmol, 0.5 eq), Pyridine (93.6 mg, 1.18 mmol, 6 eq) in DCM (3 mL) was stirred at 25° C. for 1 hour under N₂ atmosphere.

After the completion of the reaction, the mixture was quenched with H₂O (20 mL) and extracted with EtOAc (20 mL×2). The combined organic layers were washed with brine (10 mL), dried over anhydrous Na₂SO₄, filtered, and the filtrate was concentrated in vacuo. The resulting residue was purified by column chromatography to give tert-butyl (6-(((S)-4-((tert-butoxycarbonyl)amino)-3,3-difluoropentyl)oxy)pyrazin-2-yl)(1-(tert-butyl)-3-((1S,3R)-3-(((4-nitrophenoxy)carbonyl)oxy)cyclopentyl)-1H-pyrazol-5-yl)carbamate (78 mg, 49.2% yield) as a yellow oil. LCMS: 804.3 [M+H]⁺.

Step 6: Preparation of (1R,3S)-3-(5-((6-(((S)-4-amino-3,3-difluoropentyl)oxy)pyrazin-2-yl)amino)-1-(tert-butyl)-1H-pyrazol-3-yl)cyclopentyl (4-nitrophenyl) carbonate

The mixture of tert-butyl (6-(((S)-4-((tert-butoxycarbonyl)amino)-3,3-difluoropentyl)oxy)pyrazin-2-yl)(1-(tert-butyl)-3-((1S,3R)-3-(((4-nitrophenoxy)carbonyl)oxy)cyclopentyl)-1H-pyrazol-5-yl)carbamate (78 mg, 0.097 mmol, 1 eq) and TFA (66.4 mg, 0.582 mmol, 6 eq) in DCM (3 mL) was stirred at 25° C. for 3 hours under N₂ atmosphere. After the completion of the reaction, the mixture was concentrated in vacuo to give (1R,3S)-3-(5-((6-(((S)-4-amino-3,3-difluoropentyl)oxy)pyrazin-2-yl)amino)-1-(tert-butyl)-1H-pyrazol-3-yl)cyclopentyl (4-nitrophenyl) carbonate (58 mg, 0.096 mmol, crude) as a yellow oil, which was used in the next step without further purification. LCMS: 604.3 [M+H]⁺.

Step 7: Preparation of (1¹S,1³R,9S,Z)-2¹-(tert-butyl)-8,8-difluoro-9-methyl-2¹H-5,12-dioxa-3,10-diaza-4(2,6)-pyrazina-2(3,5)-pyrazola-1(1,3)-cyclopentanacyclododecaphan-11-one

A mixture of (1R,3S)-3-(5-((6-(((S)-4-amino-3,3-difluoropentyl)oxy)pyrazin-2-yl)amino)-1-(tert-butyl)-1H-pyrazol-3-yl)cyclopentyl (4-nitrophenyl) carbonate (58 mg, 0.096 mmol, 1 eq) and DIEA (72 mg, 0.557 mmol, 6 eq) in THF (56 mL) was stirred at 80° C. for 6 hours under N₂ atmosphere. After the completion of the reaction, the mixture was cooled to room temperature and concentrated in vacuo. The desired (1¹S,1³R,9S,Z)-2¹-(tert-butyl)-8,8-difluoro-9-methyl-2¹H-5,12-dioxa-3,10-diaza-4(2,6)-pyrazina-2(3,5)-pyrazola-1(1,3)-cyclopentanacyclododecaphan-11-one (43 mg, crude) was obtained as a yellow oil, which was used in the next step without further purification. LCMS: 465.3 [M+H]⁺.

Step 8: Preparation of (1¹S,1³R,9S,Z)-8,8-difluoro-9-methyl-2¹H-5,12-dioxa-3,10-diaza-4(2,6)-pyrazina-2(3,5)-pyrazola-1(1,3)-cyclopentanacyclododecaphan-11-one

A mixture of (1¹S,1³R,9S,Z)-2¹-(tert-butyl)-8,8-difluoro-9-methyl-2¹H-5,12-dioxa-3,10-diaza-4(2,6)-pyrazina-2(3,5)-pyrazola-1(1,3)-cyclopentanacyclododecaphan-11-one (43 mg, 0.093 mmol, 1 eq) in HCOOH (3 mL) was stirred at 100° C. for 1 hour under N₂ atmosphere. After the completion of the reaction, the mixture was cooled to room temperature and concentrated in vacuo. The resulting residue was purified by prep-HPLC{column: Phenomenex luna C18 150*25 mm*10 um; mobile phase: [water(FA)-ACN]; gradient:24%-54% B over 10 min and column: DAICEL CHIRALPAK AS(250 mm*30 mm, 10 um); mobile phase: [CO₂-MeOH(0.1% NH₃·H₂O)]; B %:35%, isocratic elution mode and column: Phenomenex luna C18 150*25 mm*10 um; mobile phase: [water(FA)-ACN]; gradient: 20%-50% B over 10 min} to give (1¹S,1³R,9S,Z)-8,8-difluoro-9-methyl-2¹H-5,12-dioxa-3,10-diaza-4(2,6)-pyrazina-2(3,5)-pyrazola-1(1,3)-cyclopentanacyclododecaphan-11-one (5.73 mg, 15% yield) as a white solid. LCMS: 409.3 [M+H]⁺; ¹H NMR (400 MHz, DMSO-d₆) δ 12.09-11.93 (m, 1H), 9.94-9.69 (m, 1H), 7.76 (s, 1H), 7.71-7.51 (m, 1H), 7.49 (br s, 1H), 6.62-6.40 (m, 1H), 5.13-4.96 (m, 1H), 4.66-4.46 (m, 1H), 4.20 (s, 1H), 4.13-3.90 (m, 1H), 3.25-3.18 (m, 1H), 2.48-2.38 (m, 2H), 2.11-1.98 (m, 1H), 1.98-1.84 (m, 1H), 1.83-1.60 (m, 4H), 1.17 (br d, J=6.6 Hz, 3H) ppm.

Example 44: synthesis of (1¹S,1³R,9S,Z)-7,7-difluoro-9-methyl-2¹H-5,12-dioxa-3,10-diaza-4(4,2)-pyridina-2(3,5)-pyrazola-1(1,3)-cyclopentanacyclododecaphan-11-one (compound 223)

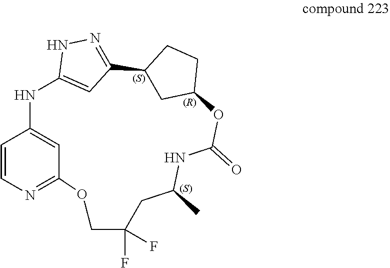

Step 1: Preparation of ethyl (4S)-4-((tert-butoxycarbonyl)amino)-2,2-difluoro-3-hydroxypentanoate

To a suspension of Zinc dust (10.2 g, 155.6 mmol, 5.2 eq) in THF (20 mL) was added dropwise a solution of tert-butyl (S)-(1-oxopropan-2-yl)carbamate (5.2 g, 30.02 mmol, 1 eq) and ethyl 2-bromo-2,2-difluoroacetate (18 g, 88.68 mmol, 3 eq) in THF (40 mL) at 20° C. under N₂ atmosphere. After the completion of the addition, the reaction mixture was warmed to 80° C. and stirred for 0.5 hour under N₂ atmosphere. The mixture was then cooled to room temperature, quenched with aqueous KHSO₄ (1 M, 100 mL) and extracted with DCM (200 mL). The combined organic layers were washed with brine (80 mL), dried over anhydrous Na₂SO₄, filtered, and the filtrate was concentrated in vacuo. The resulting residue was purified by column chromatography to give ethyl (4S)-4-((tert-butoxycarbonyl)amino)-2,2-difluoro-3-hydroxypentanoate (8.4 g, 28.25 mmol, 94.1% yield) as a yellow oil. ¹H NMR (400 MHz, CHLOROFORM-d) δ 4.82 (br d, J=7.46 Hz, 1H), 4.39-4.32 (m, 3H), 3.98 (br d, J=6.97 Hz, 1H), 1.44 (s, 9H), 1.30-1.37 (m, 6H) ppm.

Step 2: Preparation of ethyl (4S)-3-((1H-imidazole-1-carbonothioyl)oxy)-4-((tert-butoxycarbonyl)amino)-2,2-difluoropentanoate

To a solution of ethyl (4S)-4-((tert-butoxycarbonyl)amino)-2,2-difluoro-3-hydroxypentanoate (2 g, 6.73 mmol, 1 eq) in DCM (20 mL) was added di(1H-imidazol-1-yl)methanethione (1.7 g, 9.54 mmol, 1.4 eq) at 20° C., and the mixture was stirred for 12 hours under N₂ atmosphere. After the completion of the reaction, the mixture was concentrated in vacuo. The resulting residue was purified by column chromatography to afford ethyl (4S)-3-((1H-imidazole-1-carbonothioyl)oxy)-4-((tert-butoxycarbonyl)amino)-2,2-difluoropentanoate (2.3 g, 83.9% yield) as a colorless oil. LCMS: 408.2 [M+H]⁺.

Step 3: Preparation of ethyl (S)-4-((tert-butoxycarbonyl)amino)-2,2-difluoropentanoate

A mixture of ethyl (4S)-3-((1H-imidazole-1-carbonothioyl)oxy)-4-((tert-butoxycarbonyl)amino)-2,2-difluoropentanoate (2.3 g, 5.65 mmol, 1 eq), Et₃SiH (10.9 g, 93.99 mmol, 15 mL, 16.6 eq) and AlBN (1.1 g, 6.72 mmol, 1.2 eq) in anhydrous toluene (20 mL) was stirred for 12 hours at 100° C. under N₂ atmosphere. After the completion of the reaction, the mixture was cooled to room temperature. The mixture was diluted with EtOAc (20 mL), washed with water (100 mL) and brine (100 mL), and then dried over anhydrous Na₂SO₄ and filtered. The filtrate was concentrated in vacuo and the resulting residue was purified by column chromatography to afford ethyl (S)-4-((tert-butoxycarbonyl)amino)-2,2-difluoropentanoate (1.1 g, 69.3% yield) as a colorless oil. ¹H NMR (400 MHz, CDCl₃) δ 4.47 (br s, 1H), 4.31 (tq, J=3.7, 7.2 Hz, 2H), 3.95 (br s, 1H), 2.44-2.12 (m, 2H), 1.46-1.39 (m, 9H), 1.34 (t, J=7.2 Hz, 3H), 1.27-1.23 (m, 3H) ppm

Step 4: Preparation of tert-butyl (S)-(4,4-difluoro-5-hydroxypentan-2-yl)carbamate

To a solution of ethyl (S)-4-((tert-butoxycarbonyl)amino)-2,2-difluoropentanoate (0.85 g, 3.02 mmol, 1 eq) in anhydrous THF (8 mL) was added LiAlH₄ (2.5 M, 1.70 mL, 1.4 eq) at 0° C. under N₂ atmosphere, and the mixture was kept stirring for 1 hour at that temperature. After the completion of the reaction, the reaction mixture was carefully quenched with water (0.1 mL) and aqueous NaOH (0.2 mL, 1 M), followed by an additional 20 mL of water. The mixture was extracted with EtOAc (100 mL×2) and the combined organic layers were washed with brine, dried over anhydrous Na₂SO₄ and filtered. The filtrate was concentrated under reduced pressure to give tert-butyl (S)-(4,4-difluoro-5-hydroxypentan-2-yl)carbamate (687 mg, 2.87 mmol, crude) as a colorless oil, which was used into the next step without further purification.

Step 5: Preparation of tert-butyl (S)-(5-((4-bromopyridin-2-yl)oxy)-4,4-difluoropentan-2-yl)carbamate

To a mixture of tert-butyl (S)-(4,4-difluoro-5-hydroxypentan-2-yl)carbamate (687 mg, 2.87 mmol, 1 eq) and 4-bromo-2-fluoropyridine (450 mg, 2.56 mmol, 1 eq) in anhydrous THF (10 mL) was added t-BuOK (450 mg, 4 mmol, 1.4 eq) at 0° C. under N₂, and the mixture was then warmed to 20° C. and stirred for 2 hours at that temperature. After the completion of the reaction, the mixture was poured into H₂O (100 mL) and extracted with EtOAc (120 mL×2). The combined organic layers were washed with brine (100 mL), dried over anhydrous Na₂SO₄, filtered, and the filtrate was concentrated in vacuo.

The resulting residue was purified by column chromatography to give tert-butyl (S)-(5-((4-bromopyridin-2-yl)oxy)-4,4-difluoropentan-2-yl)carbamate (315 mg, 27.7% yield) as a yellow oil. ¹H NMR (400 MHz, CDCl₃) δ 7.97 (d, J=5.5 Hz, 1H), 7.08 (dd, J=1.6, 5.4 Hz, 1H), 7.04 (d, J=1.5 Hz, 1H), 4.67-4.45 (m, 3H), 4.35-4.25 (m, 1H), 4.03 (br s, 1H), 3.76-3.60 (m, 1H), 2.35-2.06 (m, 2H), 1.44-1.37 (m, 9H), 1.27-1.24 (m, 3H) ppm.

Step 6: Preparation of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-4,4-difluoropentan-2-yl)carbamate

A mixture of tert-butyl (S)-(5-((4-bromopyridin-2-yl)oxy)-4,4-difluoropentan-2-yl)carbamate (315 mg, 0.797 mmol, 1 eq), 1-(tert-butyl)-3-((1S,3R)-3-((tert-butyldimethylsilyl)oxy)cyclopentyl)-1H-pyrazol-5-amine (300 mg, 0.889 mmol, 1.1 eq), Pd₂(dba)₃ (50 mg, 0.055 mmol, 0.07 eq), Xantphos (50 mg, 0.086 mmol, 0.11 eq) and Cs₂CO₃ (450 mg, 1.38 mmol, 1.7 eq) in dioxane (8 mL) was stirred at 90° C. for 12 hours under N₂ atmosphere. After the completion of the reaction, the mixture was cooled to room temperature. The mixture was then poured into H₂O (100 mL) and extracted with EtOAc (100 mL×2). The combined organic layers were washed with H₂O (80 mL×2) and brine (80 mL), dried over anhydrous Na₂SO₄, filtered, and the filtrate was concentrated in vacuo. The resulting residue was purified by column chromatography to give tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-4,4-difluoropentan-2-yl)carbamate (510 mg, 98.2% yield) as a yellow solid. LCMS: 652.6 [M+H]⁺.

Step 7: Preparation of tert-butyl (2-(((S)-4-((tert-butoxycarbonyl)amino)-2,2-difluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-4,4-difluoropentan-2-yl)carbamate (510 mg, 0.782 mmol, 1 eq) and Boc₂O (220 mg, 1.01 mmol, 1.3 eq) in DCM (5 mL) was added DMAP (120 mg, 0.982 mmol, 1.26 eq), and the mixture was stirred at 20° C. for 2 hours under N₂ atmosphere. After the completion of the reaction, the mixture was poured into H₂O (100 mL) and extracted with EtOAc (100 mL×2). The combined organic layers were washed with brine (80 mL), dried over anhydrous Na₂SO₄, filtered, and the filtrate was concentrated in vacuo. The desired tert-butyl (2-(((S)-4-((tert-butoxycarbonyl)amino)-2,2-difluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate (550 mg, crude) was obtained as a yellow oil, which was used into the next step without further purification. LCMS: 752.6 [M+H]⁺.

Step 8: Preparation of tert-butyl (2-(((S)-4-((tert-butoxycarbonyl)amino)-2,2-difluoropentyl)oxy)pyridin-4-yl)(1-(tert-butyl)-3-((1S,3R)-3-hydroxycyclopentyl)-1H-pyrazol-5-yl)carbamate

A mixture of tert-butyl (2-(((S)-4-((tert-butoxycarbonyl)amino)-2,2-difluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate (550 mg, 0.731 mmol, 1 eq) and TBAF (1 M, 10 mL, 0.60 mmol, 13 eq) in THF (5 mL) was stirred at 20° C. for 2 hours under N₂ atmosphere. After the completion of the reaction, the mixture was poured into H₂O (100 mL) and extracted with EtOAc (100 mL×2). The combined organic layers were washed with brine (80 mL), dried over anhydrous Na₂SO₄, filtered, and the filtrate was concentrated in vacuo. The desired tert-butyl (2-(((S)-4-((tert-butoxycarbonyl)amino)-2,2-difluoropentyl)oxy)pyridin-4-yl)(1-(tert-butyl)-3-((1S,3R)-3-hydroxycyclopentyl)-1H-pyrazol-5-yl)carbamate (460 mg, crude) was obtained as a yellow oil, which was used into the next step without further purification. LCMS: 638.5 [M+H]⁺.

Step 9: Preparation of tert-butyl (2-(((S)-4-((tert-butoxycarbonyl)amino)-2,2-difluoropentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate

A mixture of tert-butyl (2-(((S)-4-((tert-butoxycarbonyl)amino)-2,2-difluoropentyl)oxy)pyridin-4-yl)(1-(tert-butyl)-3-((1S,3R)-3-hydroxycyclopentyl)-1H-pyrazol-5-yl)carbamate (460 mg, 0.721 mmol, 1 eq), 4-nitrophenyl carbonochloridate (414 mg, 2.05 mmol, 2.85 eq), DMAP (138 mg, 1.13 mmol, 1.57 eq) and Pyridine (98 mg, 1.23 mmol, 1.7 eq) in DCM (10 mL) was stirred at 20° C. for 2 hours under N₂ atmosphere. After the completion of the reaction, the mixture was concentrated in vacuo. The resulting residue was purified by prep-HPLC (TFA as an additive) to afford tert-butyl (2-(((S)-4-((tert-butoxycarbonyl)amino)-2,2-difluoropentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (550 mg, 93.3% yield) as a yellow oil. LCMS: 803.5 [M+H]⁺.

Step 10: Preparation of (1R,3S)-3-(5-((2-(((S)-4-amino-2,2-difluoropentyl)oxy)pyridin-4-yl)amino)-1-(tert-butyl)-1H-pyrazol-3-yl)cyclopentyl (4-nitrophenyl) carbonate

A mixture of tert-butyl (2-(((S)-4-((tert-butoxycarbonyl)amino)-2,2-difluoropentyl)oxy)pyridin-4-yl)(1-(tert-butyl)-3-((1S,3R)-3-(((4-nitrophenoxy)carbonyl)oxy)cyclopentyl)-1H-pyrazol-5-yl)carbamate (550 mg, 0.685 mmol, 1 eq) and TFA (3 mL) in DCM (3 mL) was stirred at 20° C. for 12 hours under N₂ atmosphere. After the completion of the reaction, the mixture was concentrated in vacuo. The resulting residue was purified by prep-HPLC (TFA as additive) to afford (1R,3S)-3-(5-((2-(((S)-4-amino-2,2-difluoropentyl)oxy)pyridin-4-yl)amino)-1-(tert-butyl)-1H-pyrazol-3-yl)cyclopentyl (4-nitrophenyl) carbonate (385 mg, 93.1% yield) as a yellow oil. LCMS: 603.5 [M+H]⁺.

Step 11: Preparation of (1¹S,1³R,9S,Z)-2¹-(tert-butyl)-7,7-difluoro-9-methyl-2¹H-5,12-dioxa-3,10-diaza-4(4,2)-pyridina-2(3,5)-pyrazola-1(1,3)-cyclopentanacyclododecaphan-11-one

The mixture of (1R,3S)-3-(5-((2-(((S)-4-amino-2,2-difluoropentyl)oxy)pyridin-4-yl)amino)-1-(tert-butyl)-1H-pyrazol-3-yl)cyclopentyl (4-nitrophenyl) carbonate (385 mg, 0.639 mmol, 1 eq) and DIEA (250 mg, 1.93 mmol, 3 eq) in THF (350 mL) was stirred for 12 hours at 80° C. under N₂ atmosphere. After the completion of the reaction, the mixture was cooled to room temperature and concentrated in vacuo. The resulting residue was purified by prep-HPLC (NH₃·H₂O as an additive) to afford (1¹S,1³R,9S,Z)-2¹-(tert-butyl)-7,7-difluoro-9-methyl-2¹H-5,12-dioxa-3,10-diaza-4(4,2)-pyridina-2(3,5)-pyrazola-1(1,3)-cyclopentanacyclododecaphan-11-one (196 mg, 66.2% yield) as a yellow solid. LCMS: 464.3 [M+H]⁺.

Step 12: Preparation of (1¹S,1³R,9S,Z)-7,7-difluoro-9-methyl-2¹H-5,12-dioxa-3,10-diaza-4(4,2)-pyridina-2(3,5)-pyrazola-1(1,3)-cyclopentanacyclododecaphan-11-one

The mixture of (1¹S,1³R,9S,Z)-2¹-(tert-butyl)-7,7-difluoro-9-methyl-2¹H-5,12-dioxa-3,10-diaza-4(4,2)-pyridina-2(3,5)-pyrazola-1(1,3)-cyclopentanacyclododecaphan-11-one (80 mg, 0.173 mmol, 1 eq) in HCOOH (4 mL) was stirred at 100° C. for 0.5 hour under N₂ atmosphere. After the completion of the reaction, the mixture was cooled to room temperature and concentrated in vacuo. The resulting residue was purified by prep-HPLC (FA as additive) to give (1¹S,1³R,9S,Z)-7,7-difluoro-9-methyl-2¹H-5,12-dioxa-3,10-diaza-4(4,2)-pyridina-2(3,5)-pyrazola-1(1,3)-cyclopentanacyclododecaphan-11-one (42.71 mg, 54.6% yield) as a white solid. LCMS: 408.2 [M+H]⁺; ¹H NMR (400 MHz, DMSO-d₆) δ 12.54-11.89 (m, 1H), 9.05 (s, 1H), 8.15 (s, 1H), 7.79 (d, J=5.7 Hz, 1H), 7.16 (br d, J=7.6 Hz, 1H), 6.51 (dd, J=1.6, 5.6 Hz, 1H), 6.39-6.28 (m, 1H), 6.05 (s, 1H), 5.05 (br s, 1H), 4.43 (td, J=11.3, 18.3 Hz, 1H), 4.31-4.14 (m, 1H), 3.80 (br d, J=3.2 Hz, 1H), 3.28-3.17 (m, 1H), 2.40-2.30 (m, 1H), 2.24 (br d, J=13.2 Hz, 1H), 2.17-2.01 (m, 2H), 1.95 (br d, J=15.2 Hz, 1H), 1.87-1.73 (m, 3H), 1.19-0.99 (m, 3H) ppm.

Example 45: synthesis of (1¹R,1³S,1⁴R,9S,Z)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one (compound 224) and (1¹S,1³R,1⁴S,9S,Z)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one (compound 225)

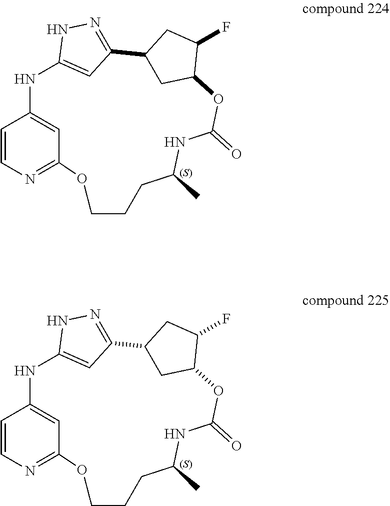

Step 1: Preparation of 3-(cyclopent-3-en-1-yl)-3-oxopropanenitrile

To a solution of MeCN (24.6 g, 599 mmol, 2.1 eq) in anhydrous THF (400 mL) was added n-BuLi (2.5 M, 297 mL, 2.6 eq) at −78° C., and the mixture was stirred for 2 hours at that temperature under N₂ atmosphere. Ethyl cyclopent-3-ene-1-carboxylate (40 g, 285.35 mmol, 1 eq) was then added to the mixture at −50° C., and the reaction mixture was stirred for an additional 4 hours at that temperature under N₂ atmosphere. After the completion of the reaction, the mixture was warmed to 0° C., quenched with aqueous NH₄Cl (500 mL) and extracted with EtOAc (500 mL×2). The combined organic layers were washed with brine (100 mL), dried over anhydrous Na₂SO₄, filtered, and the filtrate was concentrated. The desired 3-(cyclopent-3-en-1-yl)-3-oxopropanenitrile (37 g, 273.7 mmol, 95.9% yield) was obtained as a yellow oil, which was used into the next step without further purification. 1H NMR (400 MHz, CHLOROFORM-d) δ 5.65-5.62 (m, 3H), 3.56 (s, 2H), 3.45-3.34 (m, 1H), 2.67-2.60 (m, 4H) ppm.

Step 2: Preparation of 1-(tert-butyl)-3-(cyclopent-3-en-1-yl)-1H-pyrazol-5-amine

A mixture of 3-(cyclopent-3-en-1-yl)-3-oxopropanenitrile (36 g, 266.4 mmol, 1 eq), tert-butylhydrazine hydrochloride (50 g, 401.3 mmol, 1.51 eq) and DIEA (108 g, 835.6 mmol, 3.14 eq) in isopropyl alcohol (400 mL) was stirred at 80° C. for 12 hours under N₂ atmosphere. After the completion of the reaction, the mixture was cooled to room temperature and concentrated under reduced pressure. The resulting crude product was diluted with EtOAc (500 mL), washed with water (200 mL×2) and brine (200 mL×2). The organic layer was dried over anhydrous Na₂SO₄, filtered, and the filtrate was concentrated. The resulting residue was purified by column chromatography to give 1-(tert-butyl)-3-(cyclopent-3-en-1-yl)-1H-pyrazol-5-amine (36 g, 175.4 mmol, 65.8% yield) as a yellow solid. LCMS: 206.1 [M+H]⁺.

Step 3: Preparation of benzyl (1-(tert-butyl)-3-(cyclopent-3-en-1-yl)-1H-pyrazol-5-yl)carbamate

To a mixture of 1-(tert-butyl)-3-(cyclopent-3-en-1-yl)-1H-pyrazol-5-amine (34 g, 165.6 mmol, 1 eq) and NaHCO₃(70 g, 833.3 mmol, 5 eq) in MeCN (400 mL) was added CbzCl (85.0 g, 498.3 mmol, 3 eq) at 0° C. under N₂, and the mixture was then warmed to 20° C. and kept stirring for 12 hours under N₂ atmosphere. After the completion of the reaction, the mixture was filtered, rinsed with acetonitrile (50 mL), and the filtrate was concentrated under reduced pressure. The resulting crude product was diluted with EtOAc (500 mL), washed with water (200 mL) and brine (100 mL), dried over anhydrous Na₂SO₄, and filtered. The filtrate was concentrated, and the resulting residue was purified by column chromatography to give benzyl (1-(tert-butyl)-3-(cyclopent-3-en-1-yl)-1H-pyrazol-5-yl)carbamate (42 g, 74.7% yield) as a yellow oil. LCMS: 340.2 [M+H]⁺.

Step 4: Preparation of benzyl (3-((1R,3s,5S)-6-oxabicyclo[3.1.0]hexan-3-yl)-1-(tert-butyl)-1H-pyrazol-5-yl)carbamate

A mixture of benzyl (1-(tert-butyl)-3-(cyclopent-3-en-1-yl)-1H-pyrazol-5-yl)carbamate (12.5 g, 36.8 mmol, 1 eq), H₂O₂ (20.5 g, 211 mmol, 17.37 mL, 35% purity, 5.73 eq), Na₂WO₄ (1.1 g, 3.33 mmol, 0.1 eq), PhP(O)(OH)₂ (0.3 g, 1.9 mmol, 0.05 eq) and [Me(n-C₈H₁₇)₃N]HSO₄ (1.7 g, 3.65 mmol, 0.1 eq) in iPrOAc (150 mL) was stirred for 24 hours at 20° C. under N₂ atmosphere. After the completion of the reaction, the mixture was poured into aqueous Na₂SO₃ (200 mL). The organic layer was collected, and the aqueous phase was extracted with EtOAc (300 mL×2). The combined organic layers were washed with H₂O (200 mL×2) and brine (200 mL), dried over anhydrous Na₂SO₄, filtered, and the filtrate was concentrated in vacuo. The resulting residue was purified by column chromatography to give benzyl (3-((1R,3s,5S)-6-oxabicyclo[3.1.0]hexan-3-yl)-1-(tert-butyl)-1H-pyrazol-5-yl)carbamate (4.7 g, 35.9% yield) as a yellow solid. LCMS: 356.2 [M+H]⁺; ¹H NMR (400 MHz, CHLOROFORM-d) δ 7.35 (s, 5H), 6.52 (br s, 1H), 6.08-5.93 (m, 1H), 5.18 (s, 2H), 5.09 (s, 2H), 3.53 (s, 2H), 3.00-2.90 (m, 1H), 2.39 (dd, J=14.0, 7.6 Hz, 2H), 1.79 (dd, J=14.0, 10.0 Hz, 2H), 1.56 (s, 9H) ppm.

Step 5: Preparation of benzyl (1-(tert-butyl)-3-((1R,3R,4R)-3-fluoro-4-hydroxycyclopentyl)-1H-pyrazol-5-yl)carbamate

A mixture of benzyl (3-((1R,3s,5S)-6-oxabicyclo[3.1.0]hexan-3-yl)-1-(tert-butyl)-1H-pyrazol-5-yl)carbamate (3 g, 8.44 mmol, 1 eq) in triethylamine trihydrofluoride (30 mL, 184 mmol, 21.8 eq) was stirred for 12 hours at 60° C. under N₂ atmosphere. After the completion of the reaction, the mixture was cooled to room temperature. The mixture was carefully quenched with aqueous NaHCO₃(200 mL) and extracted with EtOAc (200 mL×2). The combined organic layers were washed with brine (100 mL), and dried over anhydrous Na₂SO₄, filtered, and the filtrate was concentrated in vacuo. The resulting residue was purified by column chromatography to give benzyl (1-(tert-butyl)-3-((1R,3R,4R)-3-fluoro-4-hydroxycyclopentyl)-1H-pyrazol-5-yl)carbamate (1.6 g, 50.5% yield) as a yellow solid. LCMS: 376.2 [M+H]⁺; ¹H NMR (400 MHz, CHLOROFORM-d) δ 7.37 (br s, 5H), 6.32 (br s, 1H), 6.09 (br s, 1H), 5.19 (s, 2H), 5.01-4.81 (m, 1H), 4.40 (br d, J=12.0 Hz, 1H), 3.44 (t, J=8.4 Hz, 1H), 2.69-2.50 (1H, m), 2.24-1.87 (m, 5H), 1.58 (s, 9H) ppm.

Step 6: Preparation of (1S,2R,4R)-4-(5-(((benzyloxy)carbonyl)amino)-1-(tert-butyl)-1H-pyrazol-3-yl)-2-fluorocyclopentyl 4-nitrobenzoate

To a mixture of benzyl (1-(tert-butyl)-3-((1R,3R,4R)-3-fluoro-4-hydroxycyclopentyl)-1H-pyrazol-5-yl)carbamate (1.2 g, 3.20 mmol, 1 eq), 4-nitrobenzoic acid (700 mg, 4.19 mmol, 1.3 eq) and PPh₃ (1.1 g, 4.19 mmol, 1.3 eq) in anhydrous THF (20 mL) was added DIAD (800 mg, 3.96 mmol, 1.24 eq) at 0° C., and the mixture was stirred for 1 hour at that temperature and then warmed to 20° C. and kept stirring for 12 hours under N₂ atmosphere. After the completion of the reaction, the mixture was poured into H₂O (100 mL) and extracted with EtOAc (150 mL×2). The combined organic layers were washed with brine (100 mL), dried over anhydrous Na₂SO₄, filtered, and the filtrate was concentrated in vacuo. The resulting residue was purified by prep-HPLC (FA as additive) to give (1S,2R,4R)-4-(5-(((benzyloxy)carbonyl)amino)-1-(tert-butyl)-1H-pyrazol-3-yl)-2-fluorocyclopentyl 4-nitrobenzoate (1.5 g, 89.5% yield) as a colorless oil. LCMS: 525.3 [M+H]⁺.

Step 7: Preparation of benzyl (1-(tert-butyl)-3-((1R,3R,4S)-3-fluoro-4-hydroxycyclopentyl)-1H-pyrazol-5-yl)carbamate

To a solution of (1S,2R,4R)-4-(5-(((benzyloxy)carbonyl)amino)-1-(tert-butyl)-1H-pyrazol-3-yl)-2-fluorocyclopentyl 4-nitrobenzoate (1.5 g, 2.9 mmol, 1 eq) in THF (10 mL) and MeOH (5 mL) was added aqueous LiOH (4.3 mL, 2 M, 3 eq) at 20° C. under N₂, and the reaction mixture was stirred for an additional 2 hours. After the completion of the reaction, the reaction mixture was diluted with EtOAc (300 mL) and washed sequentially with aqueous NaHCO₃(100 mL×2), water (100 mL) and brine (100 mL). The organic layer was dried over anhydrous Na₂SO₄, filtered, and the filtrate was concentrated under reduced pressure. The desired benzyl (1-(tert-butyl)-3-((1R,3R,4S)-3-fluoro-4-hydroxycyclopentyl)-1H-pyrazol-5-yl)carbamate (1 g, crude) was obtained as a yellow oil, which was used in the next step without further purification. LCMS: 376.2 [M+H]⁺.

Step 8: Preparation of benzyl (1-(tert-butyl)-3-((1R,3S,4R)-3-((tert-butyldimethylsilyl)oxy)-4-fluorocyclopentyl)-1H-pyrazol-5-yl)carbamate

To a solution of benzyl (1-(tert-butyl)-3-((1R,3R,4S)-3-fluoro-4-hydroxycyclopentyl)-1H-pyrazol-5-yl)carbamate (1 g, 2.66 mmol, 1 eq) and imidazole (550 mg, 8 mmol, 3 eq) in DCM (10 mL) was added TBSCl (620 mg, 4.1 mmol, 0.5 mL, 1.5 eq) at 0° C., and the reaction mixture was warmed to 20° C. and stirred for 12 hours at that temperature under N₂ atmosphere. After the completion of the reaction, the mixture was diluted with DCM (200 mL) and washed with water (100 mL) and brine (100 mL). The organic layer was dried over anhydrous Na₂SO₄, filtered, and the filtrate was concentrated in vacuo. The resulting residue was purified by column chromatography to afford benzyl (1-(tert-butyl)-3-((1R,3S,4R)-3-((tert-butyldimethylsilyl)oxy)-4-fluorocyclopentyl)-1H-pyrazol-5-yl)carbamate (1.2 g, 92% yield) as a colorless oil. LCMS: 490.5 [M+H]⁺; ¹H NMR (400 MHz, CDCl₃) δ 7.37 (br s, 5H), 6.33 (s, 1H), 6.14 (br s, 1H), 5.19 (s, 2H), 4.89-4.66 (m, 1H), 4.09-3.99 (m, 1H), 3.15-3.01 (m, 1H), 2.45-2.20 (m, 2H), 2.15-2.05 (m, 1H), 1.99-1.90 (m, 1H), 1.61-1.55 (m, 9H), 0.94-0.90 (m, 9H), 0.11 (d, J=4.8 Hz, 6H) ppm.

Step 9: Preparation of 1-(tert-butyl)-3-((1R,3S,4R)-3-((tert-butyldimethylsilyl)oxy)-4-fluorocyclopentyl)-1H-pyrazol-5-amine

The mixture of benzyl (1-(tert-butyl)-3-((1R,3S,4R)-3-((tert-butyldimethylsilyl)oxy)-4-fluorocyclopentyl)-1H-pyrazol-5-yl)carbamate (0.8 g, 1.63 mmol, 1 eq) and Pd/C (400 mg, 0.188 mmol, 5% purity, 0.1 eq) in THF (10 mL) and EtOAc (10 mL) was degassed and purged with H₂ for 3 times and stirred for 12 hours under H₂ atmosphere at 20° C. After the completion of the reaction, the mixture was diluted with EtOAc (10 mL), filtered, and rinsed with ethyl acetate (20 mL). The combined filtrate was concentrated to give 1-(tert-butyl)-3-((1R,3S,4R)-3-((tert-butyldimethylsilyl)oxy)-4-fluorocyclopentyl)-1H-pyrazol-5-amine (550 mg, crude) as a colorless oil, which was used in the next step without further purification. LCMS: 356.3 [M+H]⁺.

Step 10: Preparation of tert-butyl ((S)-5-((4-((1-(tert-butyl)-3-((1R,3S,4R)-3-((tert-butyldimethylsilyl)oxy)-4-fluorocyclopentyl)-1H-pyrazol-5-yl)amino)pyridin-2-yl)oxy)pentan-2-yl)carbamate

A mixture of tert-butyl (S)-(5-((4-bromopyridin-2-yl)oxy)pentan-2-yl)carbamate (200 mg, 0.557 mmol, 1 eq), 1-(tert-butyl)-3-((1R,3S,4R)-3-((tert-butyldimethylsilyl)oxy)-4-fluorocyclopentyl)-1H-pyrazol-5-amine (200 mg, 0.562 mmol, 1.01 eq), Pd₂(dba)₃ (50 mg, 0.055 mmol, 0.1 eq), XantPhos (50 mg, 0.086 mmol, 0.15 eq) and Cs₂CO₃ (300 mg, 0.921 mmol, 1.65 eq) in dioxane (8 mL) was heated and stirred at 90° C. for 12 hours under N₂ atmosphere. After the completion of the reaction, the mixture was cooled to room temperature. The mixture was diluted with H₂O (50 mL) and extracted with EtOAc (100 mL×2). The combined organic layers were washed with brine (50 mL), dried over anhydrous Na₂SO₄, filtered, and the filtrate was concentrated in vacuo. The resulting residue was purified by column chromatography to give tert-butyl ((S)-5-((4-((1-(tert-butyl)-3-((1R,3S,4R)-3-((tert-butyldimethylsilyl)oxy)-4-fluorocyclopentyl)-1H-pyrazol-5-yl)amino)pyridin-2-yl)oxy)pentan-2-yl)carbamate (315 mg, 89.3% yield) as a yellow solid. LCMS: 634.6 [M+H]⁺.

Step 11: Preparation of tert-butyl (2-(((S)-4-((tert-butoxycarbonyl)amino)pentyl)oxy)pyridin-4-yl)(1-(tert-butyl)-3-((1R,3S,4R)-3-((tert-butyldimethylsilyl)oxy)-4-fluorocyclopentyl)-1H-pyrazol-5-yl)carbamate

To a solution of tert-butyl ((S)-5-((4-((1-(tert-butyl)-3-((1R,3S,4R)-3-((tert-butyldimethylsilyl)oxy)-4-fluorocyclopentyl)-1H-pyrazol-5-yl)amino)pyridin-2-yl)oxy)pentan-2-yl)carbamate (315 mg, 0.497 mmol, 1 eq) and Boc₂O (120 mg, 0.550 mmol, 1.11 eq) in DCM (10 mL) was added DMAP (70 mg, 0.573 mmol, 1.15 eq), and the mixture was stirred at 20° C. for 2 hours under N₂ atmosphere. After the completion of the reaction, the mixture was quenched with H₂O (50 mL) and extracted with EtOAc (100 mL×2). The combined organic layers were washed with brine (50 mL×2), dried over anhydrous Na₂SO₄, filtered, and the filtrate was concentrated in vacuo. The desired tert-butyl (2-(((S)-4-((tert-butoxycarbonyl)amino)pentyl)oxy)pyridin-4-yl)(1-(tert-butyl)-3-((1R,3S,4R)-3-((tert-butyldimethylsilyl)oxy)-4-fluorocyclopentyl)-1H-pyrazol-5-yl)carbamate (345 mg, crude) was obtained as a yellow oil, which was used in the next step without further purification.

Step 12: Preparation of tert-butyl (2-(((S)-4-((tert-butoxycarbonyl)amino)pentyl)oxy)pyridin-4-yl)(1-(tert-butyl)-3-((1R,3R,4S)-3-fluoro-4-hydroxycyclopentyl)-1H-pyrazol-5-yl)carbamate

A mixture of tert-butyl (2-(((S)-4-((tert-butoxycarbonyl)amino)pentyl)oxy)pyridin-4-yl)(1-(tert-butyl)-3-((1R,3S,4R)-3-((tert-butyldimethylsilyl)oxy)-4-fluorocyclopentyl)-1H-pyrazol-5-yl)carbamate (345 mg, 0.47 mmol, 1 eq) and TBAF (1 M, 10 mL, 21 eq) in THF (5 mL) was stirred at 20° C. for 2 hours under N₂ atmosphere. After the completion of the reaction, the mixture was quenched with H₂O (50 mL) and extracted with EtOAc (80 mL×2). The combined organic layers were washed with brine (50 mL), dried over anhydrous Na₂SO₄, filtered, and the filtrate was concentrated in vacuo. The resulting residue was purified by column chromatography to give tert-butyl (2-(((S)-4-((tert-butoxycarbonyl)amino)pentyl)oxy)pyridin-4-yl)(1-(tert-butyl)-3-((1R,3R,4S)-3-fluoro-4-hydroxycyclopentyl)-1H-pyrazol-5-yl)carbamate (285 mg, 97.8% yield) as a yellow oil. LCMS: 620.6 [M+H]⁺.

Step 13: Preparation of tert-butyl (2-(((S)-4-((tert-butoxycarbonyl)amino)pentyl)oxy)pyridin-4-yl)(1-(tert-butyl)-3-((1R,3R,4S)-3-fluoro-4-(((4-nitrophenoxy)carbonyl)oxy)cyclopentyl)-1H-pyrazol-5-yl)carbamate

A mixture of tert-butyl (2-(((S)-4-((tert-butoxycarbonyl)amino)pentyl)oxy)pyridin-4-yl)(1-(tert-butyl)-3-((1R,3R,4S)-3-fluoro-4-hydroxycyclopentyl)-1H-pyrazol-5-yl)carbamate (285 mg, 0.46 mmol, 1 eq), 4-nitrophenyl carbonochloridate (280 mg, 1.39 mmol, 3 eq), DMAP (120 mg, 0.982 mmol, 2.14 eq) and Pyridine (90 mg, 1.14 mmol, 2.5 eq) in DCM (10 mL) was stirred at 20° C. for 2 hours under N₂ atmosphere. After the completion of the reaction, the mixture was quenched with H₂O (50 mL) and extracted with EtOAc (100 mL×2). The combined organic layers were washed with brine (50 mL), dried over anhydrous Na₂SO₄, filtered, and the filtrate was concentrated in vacuo. The desired tert-butyl (2-(((S)-4-((tert-butoxycarbonyl)amino)pentyl)oxy)pyridin-4-yl)(1-(tert-butyl)-3-((1R,3R,4S)-3-fluoro-4-(((4-nitrophenoxy)carbonyl)oxy)cyclopentyl)-1H-pyrazol-5-yl)carbamate (325 mg, crude) was obtained as a yellow oil, which was used in the next step without further purification. LCMS: 785.6 [M+H]⁺.

Step 14: Preparation of (1S,2R,4R)-4-(5-((2-(((S)-4-aminopentyl)oxy)pyridin-4-yl)amino)-1-(tert-butyl)-1H-pyrazol-3-yl)-2-fluorocyclopentyl (4-nitrophenyl) carbonate

A mixture of tert-butyl (2-(((S)-4-((tert-butoxycarbonyl)amino)pentyl)oxy)pyridin-4-yl)(1-(tert-butyl)-3-((1R,3R,4S)-3-fluoro-4-(((4-nitrophenoxy)carbonyl)oxy)cyclopentyl)-1H-pyrazol-5-yl)carbamate (315 mg, 0.401 mmol, 1 eq) and TFA (4.61 g, 40.4 mmol, 3 mL, 100 eq) in DCM (5 mL) was stirred at 20° C. for 2 hours. After the completion of the reaction, the mixture was concentrated in vacuo. The resulting residue was purified by prep-HPLC (TFA as an additive) to afford (1S,2R,4R)-4-(5-((2-(((S)-4-aminopentyl)oxy)pyridin-4-yl)amino)-1-(tert-butyl)-1H-pyrazol-3-yl)-2-fluorocyclopentyl (4-nitrophenyl) carbonate (270 mg, 96.3% yield) as a white solid. LCMS: 685.5 [M+H]⁺.

Step 15: Preparation of (1¹R,1³S,1⁴R,9S,Z)-2¹-(tert-butyl)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one

A mixture of (1S,2R,4R)-4-(5-((2-(((S)-4-aminopentyl)oxy)pyridin-4-yl)amino)-1-(tert-butyl)-1H-pyrazol-3-yl)-2-fluorocyclopentyl (4-nitrophenyl) carbonate (270 mg, 0.462 mmol, 1 eq) and DIEA (383 mg, 2.96 mmol, 6.4 eq) in THF (350 mL) was stirred at 80° C. for 12 hours under N₂ atmosphere. After the completion of the reaction, the mixture was cooled to room temperature and concentrated in vacuo. The desired (1¹R,1³S,1⁴R,9S,Z)-2¹-(tert-butyl)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one (205 mg, crude) was obtained as a yellow oil, which was used in the next step without further purification. LCMS: 446.3 [M+H]⁺.

Step 16: Preparation of (1¹R,1³S,1⁴R,9S,Z)-1⁴-fluoro-9-methyl-2¹H-5,12-dioxa-3,10-diaza-4(4,2)-pyridina-2(3,5)-pyrazola-1 (1,3)-cyclopentanacyclododecaphan-11-one (compound 224) and (1¹S,1³R,1⁴S,9S,Z)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one (compound 225)

A mixture of (1¹R,1³S,1⁴R,9S,Z)-2¹-(tert-butyl)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one (205 mg, 0.460 mmol, 1 eq) in HCOOH (10 mL) was stirred at 100° C. for 0.5 hour under N₂ atmosphere. After the completion of the reaction, the mixture was cooled to room temperature and concentrated in vacuo. The resulting residue was purified by prep-HPLC (column: Phenomenex luna C18 150*40 mm*15 um; mobile phase: [water(FA)-ACN]; gradient:5%-35% B over 15 min) and further by SFC (column: DAICEL CHIRALCEL OX (250 mm*30 mm, 10 um); mobile phase: [CO₂-MeOH(0.1% NH₃H₂O)]; B %:40%, isocratic elution mode) to give (11R,1³S,1⁴R,9S,Z)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one (19.5 mg, 10.9% yield) as a white solid and (1¹S,1³R,1⁴S,9S,Z)-1⁴-fluoro-9-methyl-2¹H-5,12-dioxa-3,10-diaza-4(4,2)-pyridina-2(3,5)-pyrazola-1 (1,3)-cyclopentanacyclododecaphan-11-one (14.9 mg, 8.3% yield) as a white solid.

(1¹R,1³S,1⁴R,9S,Z)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one. LCMS: 390.3 [M+H]⁺; ¹H NMR (400 MHz, DMSO-d₆) δ 12.48-11.83 (m, 1H), 8.95 (br s, 1H), 7.75 (d, J=5.6 Hz, 1H), 7.28-7.14 (m, 1H), 6.43 (br d, J=5.1 Hz, 1H), 6.34 (s, 1H), 6.11 (s, 1H), 5.19-4.96 (m, 2H), 4.15-3.95 (m, 2H), 3.74-3.62 (m, 1H), 3.27-3.11 (m, 1H), 2.47-2.31 (m, 2H), 2.18-2.02 (m, 1H), 1.82-1.70 (m, 3H), 1.65-1.44 (m, 2H), 1.32-1.18 (m, 1H), 1.06 (br d, J=6.7 Hz, 3H) ppm.

(1¹S,1³R,1⁴S,9S,Z)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one. LCMS: 390.3 [M+H]⁺; ¹H NMR (400 MHz, DMSO-d₆) δ 12.34-12.00 (m, 1H), 9.08-8.81 (m, 1H), 7.75 (br d, J=6.0 Hz, 1H), 7.26-6.72 (m, 1H), 6.47-6.34 (m, 2H), 6.18-6.04 (m, 1H), 5.29-4.93 (m, 2H), 4.17-3.93 (m, 2H), 3.70-3.38 (m, 1H), 3.29-3.09 (m, 1H), 2.42-2.27 (m, 1H), 2.18-2.01 (m, 1H), 1.95-1.51 (m, 5H), 1.42-1.23 (m, 1H), 1.15-1.02 (m, 3H) ppm.

Example 46: Synthesis of (1¹R,1³S,1⁴R,7¹S,7³R,Z)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (compound 229) and (1¹S,1³R,1⁴S,7¹R,7³S,Z)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (compound 230)

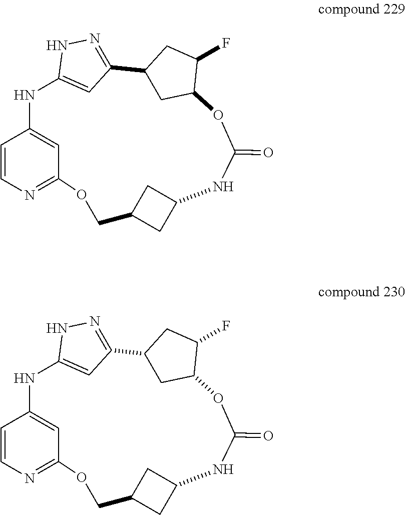

Step 1: Preparation of tert-butyl ((1R,3r)-3-(((4-((1-(tert-butyl)-3-((1R,3S,4R)-3-((tert-butyldimethylsilyl)oxy)-4-fluorocyclopentyl)-1H-pyrazol-5-yl)amino)pyridin-2-yl)oxy)methyl)cyclobutyl)carbamate

A mixture of tert-butyl ((1r,3r)-3-(((4-bromopyridin-2-yl)oxy)methyl)cyclobutyl)carbamate (392 mg, 1.1 mmol, 1 eq), 1-(tert-butyl)-3-((1R,3S,4R)-3-((tert-butyldimethylsilyl)oxy)-4-fluorocyclopentyl)-1H-pyrazol-5-amine (429.2 mg, 1.21 mmol, 1.1 eq), Pd₂(dba)₃ (100 mg, 0.109 mmol, 0.1 eq), Cs₂CO₃ (1.07 g, 3.29 mmol, 3 eq) and Xantphos (127 mg, 0.219 mmol, 0.2 eq) in dioxane (5 mL) was stirred at 90° C. for 12 hours under N₂ atmosphere. After the completion of the reaction, the mixture was cooled to room temperature. The mixture was then poured into H₂O (100 mL) and extracted with EtOAc (100 mL×2). The combined organic layers were washed with brine (100 mL), dried over anhydrous Na₂SO₄, filtered, and the filtrate was concentrated in vacuo. The resulting residue was purified by column chromatography to give tert-butyl ((1R,3r)-3-(((4-((1-(tert-butyl)-3-((1R,3S,4R)-3-((tert-butyldimethylsilyl)oxy)-4-fluorocyclopentyl)-1H-pyrazol-5-yl)amino)pyridin-2-yl)oxy)methyl)cyclobutyl)carbamate (650 mg, 93% yield) as a yellow oil. LCMS: 632.5 [M+H]⁺.

Step 2: Preparation of tert-butyl (2-(((1r,3R)-3-((tert-butoxycarbonyl)amino)cyclobutyl)methoxy)pyridin-4-yl)(1-(tert-butyl)-3-((1R,3S,4R)-3-((tert-butyldimethylsilyl)oxy)-4-fluorocyclopentyl)-1H-pyrazol-5-yl)carbamate

A mixture of tert-butyl ((1R,3r)-3-(((4-((1-(tert-butyl)-3-((1R,3S,4R)-3-((tert-butyldimethylsilyl)oxy)-4-fluorocyclopentyl)-1H-pyrazol-5-yl)amino)pyridin-2-yl)oxy)methyl)cyclobutyl)carbamate (650 mg, 1.03 mmol, 1 eq), Boc₂O (673 mg, 3.1 mmol, 3 eq) and DMAP (62 mg, 0.512 mmol, 0.5 eq) in DCM (7 mL) was stirred at 25° C. for 12 hours. After the completion of the reaction, the mixture was then poured into H₂O (150 mL) and extracted with EtOAc (200 mL×2). The combined organic layers were washed with brine (150 mL), dried over anhydrous Na₂SO₄, filtered, and the filtrate was concentrated in vacuo. The resulting residue was purified by column chromatography to give tert-butyl (2-(((1r,3R)-3-((tert-butoxycarbonyl)amino)cyclobutyl)methoxy)pyridin-4-yl)(1-(tert-butyl)-3-((1R,3S,4R)-3-((tert-butyldimethylsilyl)oxy)-4-fluorocyclopentyl)-1H-pyrazol-5-yl)carbamate (623 mg, 83% yield) as a yellow oil. LCMS: 732.4 [M+H]⁺.

Step 3: Preparation of tert-butyl (2-(((1r,3R)-3-((tert-butoxycarbonyl)amino)cyclobutyl)methoxy)pyridin-4-yl)(1-(tert-butyl)-3-((1R,3R,4S)-3-fluoro-4-hydroxycyclopentyl)-1H-pyrazol-5-yl)carbamate

A mixture of tert-butyl (2-(((1r,3R)-3-((tert-butoxycarbonyl)amino)cyclobutyl)methoxy)pyridin-4-yl)(1-(tert-butyl)-3-((1R,3S,4R)-3-((tert-butyldimethylsilyl)oxy)-4-fluorocyclopentyl)-1H-pyrazol-5-yl)carbamate (623 mg, 0.851 mmol, 1 eq) and TBAF (1 M, 5.1 mL, 6 eq) in THF (6 mL) was stirred at 25° C. for 12 hours under N₂ atmosphere. After the completion of the reaction, the reaction mixture was quenched with saturated aqueous NH₄Cl (150 mL) and extracted with EtOAc (200 mL×2). The combined organic layers were washed with brine (150 mL×1), dried over Na₂SO₄, filtered, and the filtrate was concentrated under reduced pressure. The desired tert-butyl (2-(((1r,3R)-3-((tert-butoxycarbonyl)amino)cyclobutyl)methoxy)pyridin-4-yl)(1-(tert-butyl)-3-((1R,3R,4S)-3-fluoro-4-hydroxycyclopentyl)-1H-pyrazol-5-yl)carbamate (638 mg, crude) was obtained as a yellow oil, which was used in the next step without further purification. LCMS: 618.5 [M+H]⁺.

Step 4: Preparation of tert-butyl (2-(((1r,3R)-3-((tert-butoxycarbonyl)amino)cyclobutyl)methoxy)pyridin-4-yl)(1-(tert-butyl)-3-((1R,3R,4S)-3-fluoro-4-(((4-nitrophenoxy)carbonyl)oxy)cyclopentyl)-1H-pyrazol-5-yl)carbamate

A mixture of tert-butyl (2-(((1r,3R)-3-((tert-butoxycarbonyl)amino)cyclobutyl)methoxy)pyridin-4-yl)(1-(tert-butyl)-3-((1R,3R,4S)-3-fluoro-4-hydroxycyclopentyl)-1H-pyrazol-5-yl)carbamate (618 mg, 1 mmol, 1 eq), 4-nitrophenyl carbonochloridate (605 mg, 3.00 mmol, 3 eq), DMAP (61 mg, 0.5 mmol, 0.5 eq) and Pyridine (475 mg, 6 mmol, 6 eq) in DCM (6 mL) was stirred at 25° C. for 1 hour under N₂ atmosphere. After the completion of the reaction, the mixture was then poured into H₂O (150 mL) and extracted with EtOAc (200 mL×2). The combined organic layers were washed with brine (150 mL), dried over anhydrous Na₂SO₄, filtered, and the filtrate was concentrated in vacuo. The resulting residue was purified by column chromatography to give tert-butyl (2-(((1r,3R)-3-((tert-butoxycarbonyl)amino)cyclobutyl)methoxy)pyridin-4-yl)(1-(tert-butyl)-3-((1R,3R,4S)-3-fluoro-4-(((4-nitrophenoxy)carbonyl)oxy)cyclopentyl)-1H-pyrazol-5-yl)carbamate (540 mg, 69% yield) as a yellow oil. LCMS: 783.2 [M+H]⁺.

Step 5: Preparation of (1S,2R,4R)-4-(5-((2-(((1r,3R)-3-aminocyclobutyl)methoxy)pyridin-4-yl)amino)-1-(tert-butyl)-1H-pyrazol-3-yl)-2-fluorocyclopentyl (4-nitrophenyl) carbonate

The mixture of tert-butyl (2-(((1r,3R)-3-((tert-butoxycarbonyl)amino)cyclobutyl)methoxy)pyridin-4-yl)(1-(tert-butyl)-3-((1R,3R,4S)-3-fluoro-4-(((4-nitrophenoxy)carbonyl)oxy)cyclopentyl)-1H-pyrazol-5-yl)carbamate (520 mg, 0.690 mmol, 1 eq) and TFA (454 mg, 0.3 mL, 6 eq) in DCM (5 mL) was stirred at 25° C. for 1 hour under N₂ atmosphere. After the completion of the reaction, the mixture was concentrated in vacuo to give (1S,2R,4R)-4-(5-((2-(((1r,3R)-3-aminocyclobutyl)methoxy)pyridin-4-yl)amino)-1-(tert-butyl)-1H-pyrazol-3-yl)-2-fluorocyclopentyl (4-nitrophenyl) carbonate (387 mg, crude) as a yellow oil, which was used in the next step without further purification. LCMS: 583.4 [M+H]⁺.

Step 6: Preparation of (1¹R,1³S,1⁴R,7¹S,7³R,Z)-2¹-(tert-butyl)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one

A mixture of (1S,2R,4R)-4-(5-((2-(((1r,3R)-3-aminocyclobutyl)methoxy)pyridin-4-yl)amino)-1-(tert-butyl)-1H-pyrazol-3-yl)-2-fluorocyclopentyl (4-nitrophenyl) carbonate (367 mg, 0.63 mmol, 1 eq) and DIEA (488 mg, 3.78 mmol, 6 eq) in THF (370 mL) was stirred at 60° C. for 12 hours under N₂ atmosphere. After the completion of the reaction, the mixture was cooled to room temperature, poured into H₂O (50 mL) and extracted with EtOAc (80 mL×2). The combined organic layers were washed with brine (50 mL), dried over anhydrous Na₂SO₄, filtered, and the filtrate was concentrated in vacuo. The resulting residue was purified by column chromatography to give (1¹R,1³S,1⁴R,7¹S,7³R,Z)-2¹-(tert-butyl)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (105 mg, 37% yield) as a yellow oil. LCMS: 444.3 [M+H]⁺.

Step 7: Preparation of (11R,1³S,1⁴R,7¹S,7³R,Z)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one and (1¹S,1³R,1⁴S,7¹R,7³S,Z)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one

A mixture of (1¹R,1³S,1⁴R,7¹S,7³R,Z)-2¹-(tert-butyl)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (50 mg, 0.112 mmol, 1 eq) in HCOOH (2 mL) was stirred at 100° C. for 1 hour under N₂ atmosphere. After the completion of the reaction, the mixture was cooled to room temperature and concentrated in vacuo. The resulting residue was purified by prep-HPLC (column: Phenomenex luna C18 150*25 mm*10 um; mobile phase: [water(FA)-ACN]; gradient:1%-31% B over 10 min) and further by SFC (column: DAICEL CHIRALCEL OD(250 mm*30 mm, 10 um); mobile phase: [CO₂-MeOH(0.1% NH₃H₂O)]; B %:40%, isocratic elution mode) to give (1¹R,1³S,1⁴R,7¹S,7³R,Z)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (9.81 mg, 22% yield) as an off-white solid and (1¹S,1³R,1⁴S,7¹R,7³S,Z)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (11.97 mg, 27% yield) as an off-white solid.

(1¹R,1³S,1⁴R,7¹S,7³R,Z)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one: LCMS: 388.2 [M+H]⁺; ¹H NMR (400 MHz, DMSO-d₆) δ 12.26-12.02 (m, 1H), 9.00-8.73 (m, 1H), 7.87-7.67 (m, 1H), 7.21-7.07 (m, 1H), 6.58-6.48 (m, 1H), 6.44 (br d, J=5.5 Hz, 1H), 6.13 (s, 1H), 5.32-4.92 (m, 2H), 4.40-4.21 (m, 3H), 3.25-3.19 (m, 1H), 2.41-2.01 (m, 8H), 1.77 (br d, J=13.4 Hz, 1H) ppm.

(1¹S,1³R,1⁴S,7¹R,7³S,Z)-1⁴-fluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one: LCMS: 388.2 [M+H]⁺; ¹H NMR (400 MHz, DMSO-d₆) δ 12.12 (br s, 1H), 8.89 (s, 1H), 7.75 (br d, J=5.7 Hz, 1H), 7.15 (br d, J=8.9 Hz, 1H), 6.60-6.40 (m, 2H), 6.13 (s, 1H), 5.35-4.91 (m, 2H), 4.39-4.24 (m, 3H), 3.26-3.18 (m, 1H), 2.45-1.99 (m, 8H), 1.85-1.69 (m, 1H) ppm.

Example 47: Synthesis of (1¹R,1³S,1⁴R,7¹S,7³R,E)-1⁴,2⁴-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (compound 231) and (1¹S,1³R,1⁴S,7¹R,7³S,E)-14,24-difluoro-2¹H-5,10-dioxa-3,8-diaza-4(4,2)-pyridina-2(3,5)-pyrazola-1(1 3)-cyclopentana-7(1,3)-cyclobutanacyclodecaphan-9-one (compound 232)

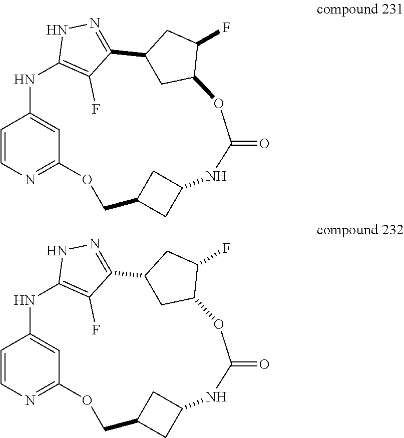

Step 1: (1¹R,1³S,14R,71S,73R, E)-21-(tert-butyl)-14,24-difluoro-21H-5,10-dioxa-3,8-diaza-4(4,2)-pyridina-2(3,5)-pyrazola-1(1,3)-cyclopentana-7(1,3)-cyclobutanacyclodecaphan-9-one

To a solution of (11R,13S,14R,71S,73R,Z)-21-(tert-butyl)-14-fluoro-21H-5,10-dioxa-3,8-diaza-4(4,2)-pyridina-2(3,5)-pyrazola-1(1,3)-cyclopentana-7(1,3)-cyclobutanacyclodecaphan-9-one (45 mg, 0.101 mmol, 1 eq) in THF (1.5 mL) was added Select-F (36 mg, 0.101 mmol, 1 eq), and the mixture was stirred at 20° C. for 16 hours under N₂ atmosphere. After the completion of the reaction, the mixture was quenched with saturated aqueous NaHCO₃(50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by prep-TLC (PE:EA=0:1) to afford (1¹R,1³S,1⁴R,7¹S,7³R,E)-2¹-(tert-butyl)-1⁴,2⁴-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one(40 mg, 85% yield) as an yellow oil. LCMS: 462.4 [M+H]⁺.

Step 2: Preparation of (1¹R,1³S,1⁴R,7¹S,7³R,E)-1⁴,2⁴-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (compound 231) and (1¹S,1³R,1⁴S,7¹R,7³S,E)-14,24-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (compound 232)

A mixture of (1¹R,1³S,1⁴R,7¹S,7³R,E)-2¹-(tert-butyl)-1⁴,2⁴-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one in HCOOH (4 mL) was stirred at 100° C. for 1 hour under N₂ atmosphere. After the completion of the reaction, the mixture was cooled to room temperature and concentrated in vacuo. The resulting residue was purified by prep-HPLC (column: Welch Xtimate C18 150*25 mm*5 um; mobile phase: [water(FA)-ACN]; gradient:2%-32% B over 10 min) and further by SFC (column: DAICEL CHIRALCEL OX (250 mm*30 mm, 10 um); mobile phase: [CO₂-i-PrOH(0.1% NH₃H₂O)]; B %:40%, isocratic elution mode) to give (1¹R,1³R,1⁴R,7¹S,7³R,E)-1⁴,2⁴-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (2.61 mg, 7.7% yield) as an off-white solid and (1¹S,1³R,1⁴S,7¹R,7³S,E)-1⁴,2⁴-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one (3.94 mg, 12% yield) as an off-white solid.

(1¹R,1³S,1⁴R,7¹S,7³R,E)-1⁴,2⁴-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one: LCMS: 406.1 [M+H]⁺; ¹H NMR (400 MHz, DMSO-d₆) δ 12.52-12.20 (m, 1H), 8.68 (s, 1H), 7.76 (br d, J=5.8 Hz, 1H), 7.11-7.04 (m, 1H), 6.45 (br d, J=5.3 Hz, 1H), 5.92-5.81 (m, 1H), 5.24-4.93 (m, 2H), 4.39-4.16 (m, 2H), 4.05 (dd, J=4.6, 11.4 Hz, 1H), 2.40-2.22 (m, 4H), 2.21-1.87 (m, 6H) ppm.

(1¹S,1³R,1⁴S,7¹R,7³S,E)-1⁴,2⁴-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one: LCMS: 406.2 [M+H]⁺; ¹H NMR (400 MHz, DMSO-d₆) δ 12.46-12.25 (m, 1H), 8.74-8.62 (m, 1H), 7.76 (d, J=6.0 Hz, 1H), 7.08 (br d, J=8.4 Hz, 1H), 6.55-6.34 (m, 1H), 5.93-5.78 (m, 1H), 5.29-4.91 (m, 2H), 4.41-4.16 (m, 2H), 4.06 (dd, J=4.5, 11.5 Hz, 1H), 2.17-1.95 (m, 6H) ppm.

Example 48: Synthesis of (1¹S,1³R,7¹R,7³S,Z)-2¹H-5,10-dioxa-3,8-diaza-4(2,6)-pyrazina-2(3,5)-pyrazola-1(1,3)-cyclopentana-7(1,3)-cyclobutanacyclodecaphan-9-one (compound 235)

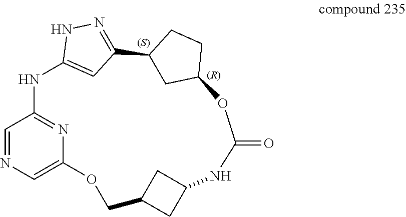

Step 1: Preparation of tert-butyl ((1r,3r)-3-(((6-bromopyrazin-2-yl)oxy)methyl)cyclobutyl)carbamate

A mixture of tert-butyl ((1r,3r)-3-(hydroxymethyl)cyclobutyl)carbamate (0.5 g, 2.48 mmol, 1 eq), 2-bromo-6-fluoropyrazine (484 mg, 2.73 mmol, 1.1 eq) and t-BuOK (418 mg, 3.73 mmol, 1.5 eq) in THF (10 mL) was stirred at 25° C. for 3 hours under N₂ atmosphere. After the completion of the reaction, the mixture was poured into H₂O (100 mL) and extracted with EtOAc (100 mL×2). The combined organic layers were washed with brine (100 mL), dried over anhydrous Na₂SO₄, filtered, and the filtrate was concentrated in vacuo. The resulting residue was purified by column chromatography to give tert-butyl ((1r,3r)-3-(((6-bromopyrazin-2-yl)oxy)methyl)cyclobutyl)carbamate (480 mg, 54% yield) as a white solid.

Step 2: Preparation of tert-butyl ((1S,3r)-3-(((6-((1-(tert-butyl)-3-((1S,3R)-3-((tert-butyldimethylsilyl)oxy)cyclopentyl)-1H-pyrazol-5-yl)amino)pyrazin-2-yl)oxy)methyl)cyclobutyl)carbamate

A mixture of tert-butyl ((1r,3r)-3-(((6-bromopyrazin-2-yl)oxy)methyl)cyclobutyl)carbamate (460 mg, 1.28 mmol, 1 eq), 1-(tert-butyl)-3-((1S,3R)-3-((tert-butyldimethylsilyl)oxy)cyclopentyl)-1H-pyrazol-5-amine (650 mg, 1.93 mmol, 1.5 eq), Cs₂CO₃ (1.26 g, 3.85 mmol, 3 eq) and Xantphos Pd G4 (247 mg, 256.82 μmol, 0.2 eq) in dioxane (10 mL) was degassed and backfilled with N₂ for 3 times, and then stirred at 75° C. for 1 hour under N₂ atmosphere. After the completion of the reaction, the mixture was cooled to room temperature. The mixture was then poured into H₂O (100 mL) and extracted with EtOAc (100 mL×2). The combined organic layers were washed with brine (100 mL), dried over anhydrous Na₂SO₄, filtered, and the filtrate was concentrated in vacuo. The resulting residue was purified by column chromatography to give tert-butyl ((1S,3r)-3-(((6-((1-(tert-butyl)-3-((1S,3R)-3-((tert-butyldimethylsilyl)oxy)cyclopentyl)-1H-pyrazol-5-yl)amino)pyrazin-2-yl)oxy)methyl)cyclobutyl)carbamate (780 mg, 98% yield) as a yellow oil. LCMS: 615.4 [M+H]⁺.

Step 3: Preparation of tert-butyl (6-(((1r,3S)-3-((tert-butoxycarbonyl)amino)cyclobutyl)methoxy)pyrazin-2-yl)(1-(tert-butyl)-3-((1S,3R)-3-((tert-butyldimethylsilyl)oxy)cyclopentyl)-1H-pyrazol-5-yl)carbamate

A mixture of tert-butyl ((1S,3r)-3-(((6-((1-(tert-butyl)-3-((1S,3R)-3-((tert-butyldimethylsilyl)oxy)cyclopentyl)-1H-pyrazol-5-yl)amino)pyrazin-2-yl)oxy)methyl)cyclobutyl)carbamate (730 mg, 1.19 mmol, 1 eq), Boc₂O (388 mg, 1.78 mmol, 1.5 eq) and DMAP (145 mg, 1.19 mmol, 1 eq) in DCM (10 mL) was stirred at 25° C. for 2 hours under N₂ atmosphere. After the completion of the reaction, the mixture was poured into H₂O (150 mL) and extracted with EtOAc (200 mL×2). The combined organic layers were washed with brine (150 mL), dried over anhydrous Na₂SO₄, filtered, and the filtrate was concentrated in vacuo. The resulting residue was purified by column chromatography to tert-butyl (6-(((1r,3S)-3-((tert-butoxycarbonyl)amino)cyclobutyl)methoxy)pyrazin-2-yl)(1-(tert-butyl)-3-((1S,3R)-3-((tert-butyldimethylsilyl)oxy)cyclopentyl)-1H-pyrazol-5-yl)carbamate (840 mg, 98% yield) as a yellow oil. LCMS: 715.5 [M+H]⁺.

Step 4: Preparation of tert-butyl (6-(((1r,3S)-3-((tert-butoxycarbonyl)amino)cyclobutyl)methoxy)pyrazin-2-yl)(1-(tert-butyl)-3-((1S,3R)-3-hydroxycyclopentyl)-1H-pyrazol-5-yl)carbamate

A mixture of tert-butyl (6-(((1r,3S)-3-((tert-butoxycarbonyl)amino)cyclobutyl)methoxy)pyrazin-2-yl)(1-(tert-butyl)-3-((1S,3R)-3-((tert-butyldimethylsilyl)oxy)cyclopentyl)-1H-pyrazol-5-yl)carbamate (840 mg, 1.17 mmol, 1 eq) and TBAF (1 M, 7 mL, 6 eq) in THF (8 mL) was stirred at 25° C. for 12 hours. After the completion of the reaction, the mixture was poured into H₂O (150 mL) and extracted with EtOAc (200 mL×2). The combined organic layers were washed with brine (150 mL), dried over anhydrous Na₂SO₄, filtered, and the filtrate was concentrated in vacuo to give tert-butyl (6-(((1r,3S)-3-((tert-butoxycarbonyl)amino)cyclobutyl)methoxy)pyrazin-2-yl)(1-(tert-butyl)-3-((1S,3R)-3-hydroxycyclopentyl)-1H-pyrazol-5-yl)carbamate (700 mg, crude) as a yellow oil, which was used in the next step without further purification.

Step 5: Preparation of tert-butyl (6-(((1r,3S)-3-((tert-butoxycarbonyl)amino)cyclobutyl)methoxy)pyrazin-2-yl)(1-(tert-butyl)-3-((1S,3R)-3-(((4-nitrophenoxy)carbonyl)oxy)cyclopentyl)-1H-pyrazol-5-yl)carbamate

A mixture of tert-butyl (6-(((1r,3S)-3-((tert-butoxycarbonyl)amino)cyclobutyl)methoxy)pyrazin-2-yl)(1-(tert-butyl)-3-((1S,3R)-3-hydroxycyclopentyl)-1H-pyrazol-5-yl)carbamate (650 mg, 1.08 mmol, 1 eq), 4-nitrophenyl carbonochloridate (654 mg, 3.25 mmol, 3 eq), DMAP (66 mg, 0.54 mmol, 0.5 eq) and Pyridine (514 mg, 6.5 mmol, 6 eq) in DCM (15 mL) was stirred at 25° C. for 1 hour. After the completion of the reaction, the mixture was poured into H₂O (150 mL) and extracted with EtOAc (200 mL×2). The combined organic layers were washed with brine (150 mL), dried over anhydrous Na₂SO₄, filtered, and the filtrate was concentrated in vacuo. The resulting residue was purified by column chromatography to give tert-butyl (6-(((1r,3S)-3-((tert-butoxycarbonyl)amino)cyclobutyl)methoxy)pyrazin-2-yl)(1-(tert-butyl)-3-((1S,3R)-3-(((4-nitrophenoxy)carbonyl)oxy)cyclopentyl)-1H-pyrazol-5-yl)carbamate (810 mg, 97% yield) as a yellow oil. LCMS: 766.4 [M+H]⁺.

Step 6: Preparation of (1R,3S)-3-(5-((6-(((1r,3S)-3-aminocyclobutyl)methoxy)pyrazin-2-yl)amino)-1-(tert-butyl)-1H-pyrazol-3-yl)cyclopentyl (4-nitrophenyl) carbonate

A mixture of tert-butyl (6-(((1r,3S)-3-((tert-butoxycarbonyl)amino)cyclobutyl)methoxy)pyrazin-2-yl)(1-(tert-butyl)-3-((1S,3R)-3-(((4-nitrophenoxy)carbonyl)oxy)cyclopentyl)-1H-pyrazol-5-yl)carbamate (810 mg, 1.06 mmol, 1 eq) and TFA (5 mL) in DCM (15 mL) was stirred at 25° C. for 12 hours. After the completion of the reaction, the mixture was concentrated in vacuo to give (1R,3S)-3-(5-((6-(((1r,3S)-3-aminocyclobutyl)methoxy)pyrazin-2-yl)amino)-1-(tert-butyl)-1H-pyrazol-3-yl)cyclopentyl (4-nitrophenyl) carbonate (1.4 g, crude) as a yellow oil, which was used in the next step without further purification. LCMS: 566.3 [M+H]⁺.

Step 7: Preparation of (1¹S,1³R,7¹R,7³S,Z)-2¹-(tert-butyl)-2¹H-5,10-dioxa-3,8-diaza-4(2,6)-pyrazina-2(3,5)-pyrazola-1(1,3)-cyclopentana-7(1,3)-cyclobutanacyclodecaphan-9-one

A mixture of (1R,3S)-3-(5-((6-(((1r,3S)-3-aminocyclobutyl)methoxy)pyrazin-2-yl)amino)-1-(tert-butyl)-1H-pyrazol-3-yl)cyclopentyl (4-nitrophenyl) carbonate (1.3 g, 2.3 mmol, 1 eq) and DIEA (2.97 g, 23 mmol, 10 eq) in DMSO (1000 mL) was stirred at 100° C. for 12 hours under N₂ atmosphere. After the completion of the reaction, the mixture was cooled to room temperature. The mixture was poured into H₂O (1000 mL) and extracted with EtOAc (800 mL×2). The combined organic layers were washed with H₂O (1000 mL×2) and brine (800 mL), dried over anhydrous Na₂SO₄, filtered, and the filtrate was concentrated in vacuo. The resulting residue was purified by column chromatography to give (1¹S,1³R,7¹R,7³S,Z)-2¹-(tert-butyl)-2¹H-5,10-dioxa-3,8-diaza-4(2,6)-pyrazina-2(3,5)-pyrazola-1(1,3)-cyclopentana-7(1,3)-cyclobutanacyclodecaphan-9-one (380 mg, 38% yield) as a yellow solid. LCMS: 427.3 [M+H]⁺.

Step 8: Preparation of (1¹S,1³R,7¹R,7³S,Z)-2¹H-5,10-dioxa-3,8-diaza-4(2,6)-pyrazina-2(3,5)-pyrazola-1(1,3)-cyclopentana-7(1,3)-cyclobutanacyclodecaphan-9-one

A mixture of (1¹S,1³R,7¹R,7³S,Z)-2¹-(tert-butyl)-2¹H-5,10-dioxa-3,8-diaza-4(2,6)-pyrazina-2(3,5)-pyrazola-1(1,3)-cyclopentana-7(1,3)-cyclobutanacyclodecaphan-9-one (120 mg, 0.281 mmol, 1 eq) in HCOOH (2 mL) was stirred at 100° C. for 1 hour under N₂ atmosphere. After the completion of the reaction, the mixture was cooled to room temperature and concentrated in vacuo. The resulting residue was purified by prep-HPLC (column: Welch Xtimate C18 150*25 mm*5 um; mobile phase: [water (FA)-ACN]; gradient:20%-50% B over 10 min) to give (1¹S,1³R,7¹R,7³S,Z)-2¹H-5,10-dioxa-3,8-diaza-4(2,6)-pyrazina-2(3,5)-pyrazola-1(1,3)-cyclopentana-7(1,3)-cyclobutanacyclodecaphan-9-one (40 mg, 38% yield) as a yellow solid. LCMS: 371.2 [M+H]⁺; ¹H NMR (400 MHz, DMSO-d₆) δ 12.35-11.75 (m, 1H), 9.74 (s, 1H), 7.77 (s, 1H), 7.41 (s, 1H), 7.16 (d, J=8.4 Hz, 1H), 6.46 (s, 1H), 5.08 (br t, J=4.1 Hz, 1H), 5.00 (br t, J=10.4 Hz, 1H), 4.51-4.37 (m, 1H), 4.14-4.02 (m, 1H), 3.28-3.20 (m, 1H), 2.46-2.30 (m, 2H), 2.14-1.82 (m, 7H), 1.74-1.50 (m, 2H) ppm.

Example 49: Synthesis of (1¹S,1³R,7¹R,7³S,E)-2⁴-fluoro-2¹H-5,10-dioxa-3,8-diaza-4(2,6)-pyrazina-2(3,5)-pyrazola-1(1,3)-cyclopentana-7(1,3)-cyclobutanacyclodecaphan-9-one (compound 236)

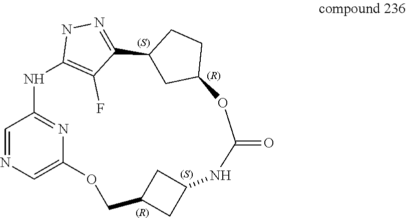

Step 1: Preparation of (1¹S,1³R,7¹R,7³S,E)-2¹-(tert-butyl)-2⁴-fluoro-2¹H-5,10-dioxa-3,8-diaza-4(2,6)-pyrazina-2(3,5)-pyrazola-1(1,3)-cyclopentana-7(1,3)-cyclobutanacyclodecaphan-9-one

To a solution of (1¹S,1³R,7¹R,7³S,Z)-2¹-(tert-butyl)-2¹H-5,10-dioxa-3,8-diaza-4(2,6)-pyrazina-2(3,5)-pyrazola-1(1,3)-cyclopentana-7(1,3)-cyclobutanacyclodecaphan-9-one (120 mg, 0.281 mmol, 1 eq) in MeCN (2.5 mL) was added Select-F (109 mg, 0.31 mmol, 1.1 eq), and the mixture was stirred at 25° C. for 12 hours under N₂ atmosphere. After the completion of the reaction, the mixture was quenched with saturated aqueous NaHCO₃(30 mL) and extracted with EtOAc (30 mL×2). The combined organic layers were washed with brine (30 mL), dried over anhydrous Na₂SO₄, filtered, and the filtrate was concentrated in vacuo. The resulting residue was purified by prep-TLC (PE:EA=1:4) to afford (1¹S,1³R,7¹R,7³S,E)-2¹-(tert-butyl)-2⁴-fluoro-2¹H-5,10-dioxa-3,8-diaza-4(2,6)-pyrazina-2(3,5)-pyrazola-1(1,3)-cyclopentana-7(1,3)-cyclobutanacyclodecaphan-9-one (40 mg, 0.09 mmol, 32% yield) as a yellow solid. LCMS: 445.2 [M+H]⁺.

Step 2: Preparation of (1¹S,1³R,7¹R,7³S,E)-2⁴-fluoro-2¹H-5,10-dioxa-3,8-diaza-4(2,6)-pyrazina-2(3,5)-pyrazola-1(1,3)-cyclopentana-7(1,3)-cyclobutanacyclodecaphan-9-one

A mixture of (1¹S,1³R,7¹R,7³S,E)-2¹-(tert-butyl)-2⁴-fluoro-2¹H-5,10-dioxa-3,8-diaza-4(2,6)-pyrazina-2(3,5)-pyrazola-1(1,3)-cyclopentana-7(1,3)-cyclobutanacyclodecaphan-9-one (40 mg, 0.09 mmol, 1 eq) in HCOOH (2 mL) was stirred at 100° C. for 1 hour under N₂ atmosphere. After the completion of the reaction, the mixture was cooled to room temperature and concentrated in vacuo. The resulting residue was purified by prep-HPLC (column: Welch Xtimate C18 150*25 mm*5 um; mobile phase: [water(FA)-ACN]; gradient:20%-50% B over 10 min) to give (1¹S,1³R,7¹R,7³S,E)-2⁴-fluoro-2¹H-5,10-dioxa-3,8-diaza-4(2,6)-pyrazina-2(3,5)-pyrazola-1(1,3)-cyclopentana-7(1,3)-cyclobutanacyclodecaphan-9-one (20 mg, 0.05 mmol, 57% yield) as a white solid.

LCMS: 389.2 [M+H]⁺; ¹H NMR (400 MHz, DMSO-d₆) δ 12.15 (br s, 1H), 9.14 (s, 1H), 7.68 (s, 1H), 7.42 (s, 1H), 7.05 (br d, J=8.8 Hz, 1H), 5.07 (br s, 1H), 4.87 (br t, J=10.4 Hz, 1H), 4.24-3.91 (m, 2H), 3.28 (br s, 1H), 2.31-1.92 (m, 6H), 1.82 (br d, J=7.2 Hz, 4H), 1.73-1.60 (m, 1H) ppm.

Example 50: Synthesis of (1¹R,1³S,1⁴R,9S,E)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one (compound 237) and (1¹S,1³R,1⁴S,9S,E)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one (compound 238)

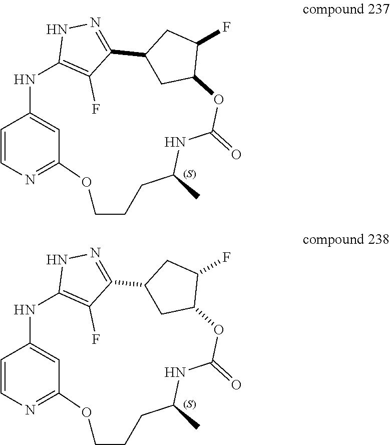

Step 1: Preparation of (11R,13S,14R,9S, E)-21-(tert-butyl)-14,24-difluoro-9-methyl-21H-5,12-dioxa-3,10-diaza-4(4,2)-pyridina-2(3,5)-pyrazola-1 (1,3)-cyclopentanacyclododecaphan-11-one

To a solution of (1¹R,1³S,1⁴R,9S,Z)-2¹-(tert-butyl)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one (140 mg, 0.314 mmol, 1 eq) in acetonitrile (3 mL) was added Select-F (122 mg, 0.345 mmol, 1.1 eq) under N₂ atmosphere, and the mixture was stirred at 25° C. for 12 hours. After the completion of the reaction, the mixture was quenched with saturated aqueous NaHCO₃(50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by column chromatography to afford (11R,1³S,1⁴R,9S,E)-2¹-(tert-butyl)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one (110 mg, 0.237 mmol, 75% yield) as a colorless oil. LCMS: 464.2 [M+H]⁺.

Step 2: Preparation of (1¹R,1³S,1⁴R,9S,E)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one (compound 237) and (1¹S,1³R,1⁴S,9S,E)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one (compound 238)

A mixture of (1¹R,1³S,1⁴R,9S,E)-2¹-(tert-butyl)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one (100 mg, 0.216 mmol, 1 eq) in HCOOH (2 mL) was stirred at 100° C. for 1 hour under N₂ atmosphere. After the completion of the reaction, the mixture was cooled to room temperature and concentrated in vacuo. The resulting residue was purified by prep-HPLC (column: Welch Xtimate C18 150*25 mm*5 um; mobile phase: [water(FA)-ACN]; gradient:5%-35% B over 10 min) and further by SFC (column: DAICEL CHIRALCEL OX (250 mm*30 mm, 10 um); mobile phase: [CO₂-MeOH(0.1% NH₃H₂O)]; B %:35%, isocratic elution mode) to give (1¹R,1³S,1⁴R,9S,E)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one (18.5 mg, 41% yield) as a white solid (absolute configuration is randomly assigned) and (1¹S,1³R,1⁴S,9S,E)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one (18 mg, 37% yield) as a white solid(absolute configuration is randomly assigned).

(1¹R,1³S,1⁴R,9S,E)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one: LCMS: 408.2 [M+H]⁺; 1H NMR (400 MHz, DMSO-d₆) δ 12.54-12.21 (m, 1H), 8.78-8.68 (m, 1H), 7.75 (d, J=5.6 Hz, 1H), 6.96 (d, J=8.8 Hz, 1H), 6.43 (d, J=5.2 Hz, 1H), 5.84 (s, 1H), 5.18-4.99 (m, 1H), 4.92 (d, J=4.0 Hz, 1H), 3.95 (t, J=6.8 Hz, 2H), 3.68-3.55 (m, 1H), 3.29-3.19 (m, 1H), 2.46-2.33 (m, 3H), 1.95-1.82 (m, 1H), 1.75-1.62 (m, 2H), 1.54-1.42 (m, 2H), 1.02 (d, J=6.4 Hz, 3H) ppm.

(1¹S,1³R,1⁴S,9S,E)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one: LCMS: 408.2 [M+H]⁺; ¹H NMR (400 MHz, DMSO-d₆) δ 12.69-12.07 (m, 1H), 8.80 (s, 1H), 7.76 (d, J=5.2 Hz, 1H), 6.83-6.68 (m, 1H), 6.43 (d, J=5.2 Hz, 1H), 5.92-5.71 (m, 1H), 5.35-4.90 (m, 2H), 3.98 (d, J=7.2 Hz, 2H), 3.53-3.14 (m, 3H), 2.44-2.28 (m, 2H), 1.97-1.80 (m, 1H), 1.74-1.48 (m, 3H), 1.33-1.20 (m, 1H), 1.14-0.98 (m, 3H) ppm.

Example 51: Synthesis of (1¹S,1³R,9S,Z)-9-methyl-2¹H-5,12-dioxa-3,10-diaza-4(4,2)-pyrimidina-2(3,5)-pyrazola-1(1,3)-cyclopentanacyclododecaphan-11-one (compound 241)

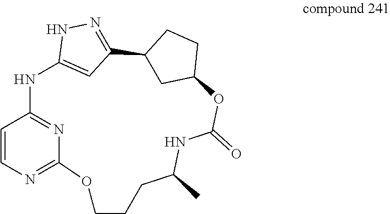 [SMILES: C[C@H]1CCCOc2nccc(n2)Nc2cc(n[nH]2)[C@H]2CC[C@H](C2)OC(=O)N1]

Step 1: Preparation of tert-butyl (S)-(5-((4-bromopyrimidin-2-yl)oxy)pentan-2-yl)carbamate

A mixture of 4-bromo-2-(methylsulfonyl)pyrimidine (371 mg, 1.57 mmol, 1 eq), tert-butyl (S)-(5-hydroxypentan-2-yl)carbamate (350 mg, 1.72 mmol, 1.1 eq) and t-BuOK (1 M, 1.88 mL, 1.2 eq) in THF (5 mL) was stirred at −78° C. for 1 hour under N₂ atmosphere. After the completion of the reaction, the mixture was quenched with NH₄Cl (50 mL) and extracted with EtOAc (50 mL×3). The combined organic layers were washed with brine (50 mL), dried over anhydrous Na₂SO₄, filtered, and the filtrate was concentrated in vacuo. The resulting residue was purified by column chromatography to give tert-butyl (S)-(5-((4-bromopyrimidin-2-yl)oxy)pentan-2-yl)carbamate (500 mg, 88% yield) as a yellow oil.

Step 2: Preparation of tert-butyl ((S)-5-((4-((1-(tert-butyl)-3-((1S,3R)-3-((tert-butyldimethylsilyl)oxy)cyclopentyl)-1H-pyrazol-5-yl)amino)pyrimidin-2-yl)oxy)pentan-2-yl)carbamate

A mixture of tert-butyl (S)-(5-((4-bromopyrimidin-2-yl)oxy)pentan-2-yl)carbamate (237 mg, 0.658 mmol, 1 eq), 1-(tert-butyl)-3-((1S,3R)-3-((tert-butyldimethylsilyl)oxy)cyclopentyl)-1H-pyrazol-5-amine (333 mg, 0.986 mmol, 1.5 eq), Cs₂CO₃ (643 mg, 1.97 mmol, 3 eq) and Xantphos-Pd G4 (127 mg, 0.132 mmol, 0.2 eq) in dioxane (5 mL) was degassed and backfilled with N₂ for 3 times, and then was stirred at 75° C. for 1 hour under N₂ atmosphere. After the completion of the reaction, the mixture was cooled to room temperature. The mixture was then poured into H₂O (50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by column chromatography to give tert-butyl ((S)-5-((4-((1-(tert-butyl)-3-((1S,3R)-3-((tert-butyldimethylsilyl)oxy)cyclopentyl)-1H-pyrazol-5-yl)amino)pyrimidin-2-yl)oxy)pentan-2-yl)carbamate (467 mg, 97% yield) as a yellow oil. LCMS: 617.4 [M+H]⁺.

Step 3: Preparation of tert-butyl (2-(((S)-4-((tert-butoxycarbonyl)amino)pentyl)oxy)pyrimidin-4-yl)(1-(tert-butyl)-3-((1S,3R)-3-((tert-butyldimethylsilyl)oxy)cyclopentyl)-1H-pyrazol-5-yl)carbamate

A mixture of tert-butyl ((S)-5-((4-((1-(tert-butyl)-3-((1S,3R)-3-((tert-butyldimethylsilyl)oxy)cyclopentyl)-1H-pyrazol-5-yl)amino)pyrimidin-2-yl)oxy)pentan-2-yl)carbamate (417 mg, 0.675 mmol, 1 eq), Boc₂O (221 mg, 1.01 mmol, 1.5 eq) and DMAP (41 mg, 0.338 mmol, 0.5 eq) in DCM (5 mL) was stirred at 25° C. for 12 hours under N₂ atmosphere. After the completion of the reaction, the mixture was poured into H₂O (50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by column chromatography to give tert-butyl (2-(((S)-4-((tert-butoxycarbonyl)amino)pentyl)oxy)pyrimidin-4-yl)(1-(tert-butyl)-3-((1S,3R)-3-((tert-butyldimethylsilyl)oxy)cyclopentyl)-1H-pyrazol-5-yl)carbamate (422 mg, 87% yield) as a yellow oil. LCMS: 717.4 [M+H]⁺.

Step 4: Preparation of tert-butyl (2-(((S)-4-((tert-butoxycarbonyl)amino)pentyl)oxy)pyrimidin-4-yl)(1-(tert-butyl)-3-((1S,3R)-3-hydroxycyclopentyl)-1H-pyrazol-5-yl)carbamate

A mixture of tert-butyl (2-(((S)-4-((tert-butoxycarbonyl)amino)pentyl)oxy)pyrimidin-4-yl)(1-(tert-butyl)-3-((1S,3R)-3-((tert-butyldimethylsilyl)oxy)cyclopentyl)-1H-pyrazol-5-yl)carbamate (400 mg, 0.560 mmol, 1 eq) and TBAF (1 M, 5 mL) in THF (5 mL) was stirred at 25° C. for 12 hours under N₂ atmosphere. After the completion of the reaction, the mixture was poured into H₂O (50 mL) and extracted with EtOAc (50 mL×2). The combined organic layers were washed with brine (50 mL), dried over anhydrous Na₂SO₄, filtered, and the filtrate was concentrated under reduced pressure to give tert-butyl (2-(((S)-4-((tert-butoxycarbonyl)amino)pentyl)oxy)pyrimidin-4-yl)(1-(tert-butyl)-3-((1S,3R)-3-hydroxycyclopentyl)-1H-pyrazol-5-yl)carbamate (487 mg, crude) as a yellow oil, which was used in the next step without further purification.

Step 5: Preparation of tert-butyl (2-(((S)-4-((tert-butoxycarbonyl)amino)pentyl)oxy)pyrimidin-4-yl)(1-(tert-butyl)-3-((1S,3R)-3-(((4-nitrophenoxy)carbonyl)oxy)cyclopentyl)-1H-pyrazol-5-yl)carbamate

A mixture of tert-butyl (2-(((S)-4-((tert-butoxycarbonyl)amino)pentyl)oxy)pyrimidin-4-yl)(1-(tert-butyl)-3-((1S,3R)-3-hydroxycyclopentyl)-1H-pyrazol-5-yl)carbamate (467 mg, 0.775 mmol, 1 eq), 4-nitrophenyl carbonochloridate (468 mg, 2.32 mmol, 3 eq), DMAP (47 mg, 0.387 mmol, 0.5 eq) and Pyridine (367 mg, 4.65 mmol, 6 eq) in DCM (5 mL) was stirred at 25° C. for 2 hours under N₂ atmosphere. After the completion of the reaction, the mixture was then poured into H₂O (50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by column chromatography to give tert-butyl (2-(((S)-4-((tert-butoxycarbonyl)amino)pentyl)oxy)pyrimidin-4-yl)(1-(tert-butyl)-3-((1S,3R)-3-(((4-nitrophenoxy)carbonyl)oxy)cyclopentyl)-1H-pyrazol-5-yl)carbamate (406 mg, 0.528 mmol, 68% yield) as a yellow oil. LCMS: 768.4 [M+H]⁺.

Step 6: Preparation of (1R,3S)-3-(5-((2-(((S)-4-aminopentyl)oxy)pyrimidin-4-yl)amino)-1-(tert-butyl)-1H-pyrazol-3-yl)cyclopentyl (4-nitrophenyl) carbonate

A mixture of tert-butyl (2-(((S)-4-((tert-butoxycarbonyl)amino)pentyl)oxy)pyrimidin-4-yl)(1-(tert-butyl)-3-((1S,3R)-3-(((4-nitrophenoxy)carbonyl)oxy)cyclopentyl)-1H-pyrazol-5-yl)carbamate (386 mg, 0.502 mmol, 1 eq) and TFA (171 mg, 1.51 mmol, 3 eq) in DCM (4 mL) was stirred at 25° C. for 2 hours under N₂ atmosphere. After the completion of the reaction, the mixture was concentrated in vacuo. The desired (1R,3S)-3-(5-((2-(((S)-4-aminopentyl)oxy)pyrimidin-4-yl)amino)-1-(tert-butyl)-1H-pyrazol-3-yl)cyclopentyl (4-nitrophenyl) carbonate (285 mg, crude product) was obtained as a yellow oil, which was used into the next step without further purification.

Step 7: Preparation of (1¹S,1³R,9S,Z)-2¹-(tert-butyl)-9-methyl-2¹H-5,12-dioxa-3,10-diaza-4(4,2)-pyrimidina-2(3,5)-pyrazola-1(1,3)-cyclopentanacyclododecaphan-11-one

A mixture of (1R,3S)-3-(5-((2-(((S)-4-aminopentyl)oxy)pyrimidin-4-yl)amino)-1-(tert-butyl)-1H-pyrazol-3-yl)cyclopentyl (4-nitrophenyl) carbonate (285 mg, 0.502 mmol, 1 eq) and DIEA (324 mg, 2.51 mmol, 5 eq) in DMSO (280 mL) was stirred at 80° C. for 3 hours under N₂ atmosphere. After the completion of the reaction, the mixture was then poured into H₂O (50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by column chromatography to give (1¹S,1³R,9S,Z)-2¹-(tert-butyl)-9-methyl-2¹H-5,12-dioxa-3,10-diaza-4(4,2)-pyrimidina-2(3,5)-pyrazola-1(1,3)-cyclopentanacyclododecaphan-11-one (201 mg, 93% yield) as a yellow oil. LCMS: 429.3 [M+H]⁺.

Step 8: Preparation of (1¹S,1³R,9S,Z)-9-methyl-2¹H-5,12-dioxa-3,10-diaza-4(4,2)-pyrimidina-2(3,5)-pyrazola-1(1,3)-cyclopentanacyclododecaphan-11-one

A mixture of (1¹S,1³R,9S,Z)-2¹-(tert-butyl)-9-methyl-2¹H-5,12-dioxa-3,10-diaza-4(4,2)-pyrimidina-2(3,5)-pyrazola-1(1,3)-cyclopentanacyclododecaphan-11-one (54 mg, 0.126 mmol, 1 eq) in HCOOH (2 mL) was stirred at 100° C. for 1 hour under N₂ atmosphere. After the completion of the reaction, the mixture was cooled to room temperature and concentrated in vacuo. The resulting residue was purified by prep-HPLC (column: Welch Xtimate C18 150*25 mm*5 um; mobile phase: [water (FA)-ACN]; gradient:8%-38% B over 10 min}) to give (1¹S,1³R,9S,Z)-9-methyl-2¹H-5,12-dioxa-3,10-diaza-4(4,2)-pyrimidina-2(3,5)-pyrazola-1(1,3)-cyclopentanacyclododecaphan-11-one (7.3 mg, 15% yield) as a white solid. LCMS: 373.2 [M+H]⁺; ¹H NMR (400 MHz, DMSO-d₆) δ 12.35-11.77 (m, 1H), 10.10-9.85 (m, 1H), 8.19 (s, 1H), 7.96 (d, J=5.5 Hz, 1H), 7.02-6.66 (m, 1H), 6.62 (s, 1H), 6.37 (d, J=5.6 Hz, 1H), 5.08-4.97 (m, 1H), 4.61-4.38 (m, 1H), 4.15-3.93 (m, 1H), 3.73-3.51 (m, 1H), 3.21-3.16 (m, 1H), 2.05-1.62 (m, 8H), 1.53-1.24 (m, 1H), 1.09-0.97 (m, 3H) ppm.

Example 52: Synthesis of (1¹S,1³R,9S,E)-2⁴-fluoro-9-methyl-2¹H-5,12-dioxa-3,10-diaza-4(4,2)-pyrimidina-2(3,5)-pyrazola-1(1,3)-cyclopentanacyclododecaphan-11-one (compound 256)

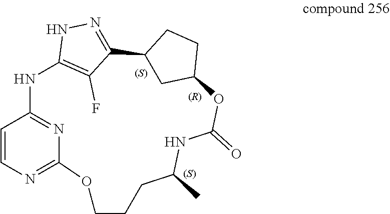

Step 1: Preparation of (1¹S,1³R,9S,E)-2¹-(tert-butyl)-2⁴-fluoro-9-methyl-2¹H-5,12-dioxa-3,10-diaza-4(4,2)-pyrimidina-2(3,5)-pyrazola-1(1,3)-cyclopentanacyclododecaphan-11-one

To a solution (1¹S,1³R,9S,Z)-2¹-(tert-butyl)-9-methyl-2¹H-5,12-dioxa-3,10-diaza-4(4,2)-pyrimidina-2(3,5)-pyrazola-1(1,3)-cyclopentanacyclododecaphan-11-one (80 mg, 0.187 mmol, 1 eq) in ACN (2 mL) was added Select-F (73 mg, 0.205 mmol, 1.1 eq), and the mixture was stirred at 25° C. for 12 hours under N₂ atmosphere. After the completion of the reaction, the mixture was quenched with saturated aqueous NaHCO₃(20 mL) and extracted with EtOAc (20 mL×2). The combined organic layers were washed with brine (20 mL), dried over anhydrous Na₂SO₄, filtered and the filtrate was concentrated in vacuo. The resulting residue was purified by prep-HPLC (column: Welch Xtimate C18 150*25 mm*5 um; mobile phase: [water(FA)-ACN]; gradient:18%-48% B over 10 min) to give (1¹S,1³R,9S,E)-2¹-(tert-butyl)-2⁴-fluoro-9-methyl-2¹H-5,12-dioxa-3,10-diaza-4(4,2)-pyrimidina-2(3,5)-pyrazola-1(1,3)-cyclopentanacyclododecaphan-11-one (47 mg, 56% yield) as a yellow oil. LCMS: 447.2 [M+H]⁺.

Step 2: Preparation of (1¹S,1³R,9S,E)-2⁴-fluoro-9-methyl-2¹H-5,12-dioxa-3,10-diaza-4(4,2)-pyrimidina-2(3,5)-pyrazola-1 (1,3)-cyclopentanacyclododecaphan-11-one

A mixture of (1¹S,1³R,9S,E)-2¹-(tert-butyl)-2⁴-fluoro-9-methyl-2¹H-5,12-dioxa-3,10-diaza-4(4,2)-pyrimidina-2(3,5)-pyrazola-1(1,3)-cyclopentanacyclododecaphan-11-one (46 mg, 0.103 mmol, 1 eq) in HCOOH (1 mL) was stirred at 100° C. for 1 hour under N₂ atmosphere. After the completion of the reaction, the mixture was cooled to room temperature and concentrated in vacuo. The resulting residue was purified by prep-HPLC (column: Welch Xtimate C18 150*25 mm*5 um; mobile phase: [water(FA)-ACN]; gradient:8%-38% B over 10 min) to give (1¹S,1³R,9S,E)-2⁴-fluoro-9-methyl-2¹H-5,12-dioxa-3,10-diaza-4(4,2)-pyrimidina-2(3,5)-pyrazola-1(1,3)-cyclopentanacyclododecaphan-11-one (12.5 mg, 0.032 mmol, 31% yield) as a white solid. LCMS: 391.1 [M+H]⁺; ¹H NMR (400 MHz, DMSO-d₆) δ 12.61-11.90 (m, 1H), 9.77-9.31 (m, 1H), 8.00 (br d, J=5.5 Hz, 1H), 6.83-6.47 (m, 1H), 6.32 (d, J=5.6 Hz, 1H), 5.10-4.88 (m, 1H), 4.64-4.17 (m, 1H), 3.91-3.72 (m, 1H), 3.71-3.35 (m, 1H), 3.26-3.18 (m, 1H), 2.44-2.29 (m, 1H), 2.25-1.95 (m, 2H), 1.94-1.64 (m, 4H), 1.60-1.08 (m, 3H), 1.06-0.86 (m, 3H) ppm.

Example 53: Synthesis of (1¹R,1³S,1⁴R,9S,Z)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (Compound 226) and (1¹S,1³R,1⁴S,9S,Z)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (compound 227)

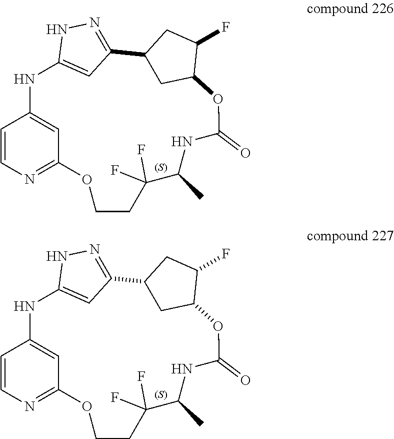

Step 1: Preparation of (S,E)-N-ethylidene-2-methylpropane-2-sulfinamide

To a solution of (S)-2-methylpropane-2-sulfinamide (5 g, 41.25 mmol, 1 eq) in DCM (50 mL) was added MgSO₄ (9.93 g, 82.5 mmol, 2 eq), and the mixture was stirred at 20° C. for 5 minutes, followed by the addition of acetaldehyde (5 M, 16.50 mL, 2 eq) and PPTS (311 mg, 1.24 mmol, 0.03 eq). The resulting mixture was stirred at 20° C. for 24 hours. After the completion of the reaction, the reaction was filtered, and the filtrate was concentrated under reduced pressure. The resulting residue was purified by column chromatography to give (S,E)-N-ethylidene-2-methylpropane-2-sulfinamide (6 g, 40.7 mmol, 98% yield) as a yellow oil. LCMS: 148.2 [M+H]⁺.

Step 2: Preparation of (S)—N—((S)-3,3-difluoropent-4-en-2-yl)-2-methylpropane-2-sulfinamide

To a solution of (S,E)-N-ethylidene-2-methylpropane-2-sulfinamide (5 g, 33.96 mmol, 1 eq) and 3-bromo-3,3-difluoro-prop-1-ene (6.4 g, 40.7 mmol, 1.2 eq) in DMF (27.5 mL) was added Indium (5.85 g, 50.94 mmol, 1.5 eq) at 0° C., and the mixture was then raised to 80° C. and stirred for 12 hours under N₂ atmosphere. After the completion of the reaction, the mixture was cooled to room temperature. The mixture was then poured into H₂O (150 mL) and extracted with EtOAc (200 mL×2). The combined organic layers were washed with brine (150 mL), dried over anhydrous Na₂SO₄, filtered, and the filtrate was concentrated in vacuo. The resulting residue was purified by column chromatography to give (S)—N—((S)-3,3-difluoropent-4-en-2-yl)-2-methylpropane-2-sulfinamide (2.2 g, 29% yield) as a yellow solid. LCMS: 226.2 [M+H]⁺.

Step 3: Preparation of (S)—N—((S)-3,3-difluoro-5-hydroxypentan-2-yl)-2-methylpropane-2-sulfinamide

To a cooled solution of (S)—N—((S)-3,3-difluoropent-4-en-2-yl)-2-methylpropane-2-sulfinamide (1 g, 4.44 mmol, 1 eq) in THF (20 mL) was added 9-BBN (0.5 M, 17.7 mL, 2 eq) dropwise at 0° C. After the completion of the addition, the mixture was stirred at 50° C. for 10 hours under N₂ atmosphere. The mixture was then cooled to 0° C., quenched with aqueous NaOH (6 mL), followed by the addition of H₂O₂ (6 mL, 30% purity) and stirred at 40° C. for 10 hours. After the completion of the reaction, the mixture was cooled to room temperature, quenched with sat·aq·Na₂SO₃ (50 mL) and extracted with EtOAc (50 mL×3). The combined organic layers were dried over anhydrous Na₂SO₄, filtered, and the filtrate was concentrated in vacuo. The resulting residue was purified by column chromatography to give (S)—N—((S)-3,3-difluoro-5-hydroxypentan-2-yl)-2-methylpropane-2-sulfinamide (864 mg, 80% yield) as a colorless oil. LCMS: 244.5 [M+H]⁺.

Step 4: Preparation of (S)—N—((S)-5-((4-bromopyridin-2-yl)oxy)-3,3-difluoropentan-2-yl)-2-methylpropane-2-sulfinamide

To a solution of (S)—N—((S)-3,3-difluoro-5-hydroxypentan-2-yl)-2-methylpropane-2-sulfinamide (864 mg, 3.55 mmol, 1 eq) and 4-bromo-2-fluoropyridine (625 mg, 3.55 mmol, 1 eq) in THF (17 mL) was added t-BuOK (398 mg, 3.55 mmol, 1 eq) at 0° C., and the mixture was stirred at 20° C. for 1 hour under N₂ atmosphere. After the completion of the reaction, the mixture was quenched with NH₄Cl (120 mL) and extracted with EtOAc (100 mL×3). The combined organic layers were washed with brine (100 mL), dried over anhydrous Na₂SO₄, filtered, and the filtrate was concentrated in vacuo. The resulting residue was purified by column chromatography to give (S)—N—((S)-5-((4-bromopyridin-2-yl)oxy)-3,3-difluoropentan-2-yl)-2-methylpropane-2-sulfinamide (500 mg, 88% yield) as a colorless oil. LCMS: 399.0 [M+H]⁺; LCMS: 401.0 [M+H+2]⁺.

Step 5: Preparation of (S)-5-((4-bromopyridin-2-yl)oxy)-3,3-difluoropentan-2-amine

The solution of (S)—N—((S)-5-((4-bromopyridin-2-yl)oxy)-3,3-difluoropentan-2-yl)-2-methylpropane-2-sulfinamide (990 mg, 2.48 mmol, 1 eq) in HCl/dioxane (2 M, 20 mL, 16 eq) was stirred at 20° C. for 1 hour. After the completion of the reaction, the mixture was concentrated in vacuo to give (S)-5-((4-bromopyridin-2-yl)oxy)-3,3-difluoropentan-2-amine (822 mg, crude, HCl) as a white solid, which was used into the next step without further purification.

Step 6: Preparation of tert-butyl (S)-(5-((4-bromopyridin-2-yl)oxy)-3,3-difluoropentan-2-yl)carbamate

To a solution of (S)-5-((4-bromopyridin-2-yl)oxy)-3,3-difluoropentan-2-amine (822 mg, 2.48 mmol, 1 eq, HCl) in DCM (20 mL) was added Boc₂O (1.08 g, 4.96 mmol, 2 eq) and TEA (1.25 g, 12.4 mmol, 5 eq), and the mixture was stirred at 20° C. for 12 hours. After the completion of the reaction, the mixture was concentrated in vacuo. The resulting residue was purified by column chromatography to give tert-butyl (S)-(5-((4-bromopyridin-2-yl)oxy)-3,3-difluoropentan-2-yl)carbamate (500 mg, 1.27 mmol, 51% yield) as a colorless oil. LCMS: 395.0; 397.0 [M+H]⁺. ¹H NMR (400 MHz, CHLOROFORM-d) δ 7.97 (d, J=5.5 Hz, 1H), 7.06-7.01 (m, 1H), 6.97 (s, 1H), 4.65 (br d, J=8.6 Hz, 1H), 4.54 (t, J=6.7 Hz, 2H), 4.23-4.05 (m, 1H), 2.52-2.31 (m, 2H), 1.46 (s, 9H), 1.26 (d, J=6.8 Hz, 3H) ppm

Step 7: Preparation of tert-butyl ((S)-5-((4-((1-(tert-butyl)-3-((1R,3S,4R)-3-((tert-butyldimethylsilyl)oxy)-4-fluorocyclopentyl)-1H-pyrazol-5-yl)amino)pyridin-2-yl)oxy)-3,3-difluoropentan-2-yl)carbamate

A mixture of tert-butyl (S)-(5-((4-bromopyridin-2-yl)oxy)-3,3-difluoropentan-2-yl)carbamate (480 mg, 1.21 mmol, 1 eq), 1-(tert-butyl)-3-((1R,3S,4R)-3-((tert-butyldimethylsilyl)oxy)-4-fluorocyclopentyl)-1H-pyrazol-5-amine (475 mg, 1.34 mmol, 1.1 eq), Xantphos (141 mg, 0.243 mmol, 0.2 eq), Pd₂(dba)₃ (111 mg, 0.121 mmol, 0.1 eq) and Cs₂CO₃ (791 mg, 2.43 mmol, 2 eq) in dioxane (10 mL) was degassed and backfilled with N₂ for 3 times, and then stirred at 90° C. for 3 hours under N₂ atmosphere. After the completion of the reaction, the mixture was cooled to room temperature. The mixture was then poured into H₂O (100 mL) and extracted with EtOAc (100 mL×2). The combined organic layers were washed with brine (100 mL), dried over anhydrous Na₂SO₄, filtered, and the filtrate was concentrated in vacuo. The resulting residue was purified by column chromatography to give tert-butyl ((S)-5-((4-((1-(tert-butyl)-3-((1R,3S,4R)-3-((tert-butyldimethylsilyl)oxy)-4-fluorocyclopentyl)-1H-pyrazol-5-yl)amino)pyridin-2-yl)oxy)-3,3-difluoropentan-2-yl)carbamate (810 mg, 99% yield) as a yellow oil. LCMS: 670.3 [M+H]⁺.

Step 8: Preparation of tert-butyl (2-(((S)-4-((tert-butoxycarbonyl)amino)-3,3-difluoropentyl)oxy)pyridin-4-yl)(1-(tert-butyl)-3-((1R,3S,4R)-3-((tert-butyldimethylsilyl)oxy)-4-fluorocyclopentyl)-1H-pyrazol-5-yl)carbamate

A mixture of tert-butyl ((S)-5-((4-((1-(tert-butyl)-3-((1R,3S,4R)-3-((tert-butyldimethylsilyl)oxy)-4-fluorocyclopentyl)-1H-pyrazol-5-yl)amino)pyridin-2-yl)oxy)-3,3-difluoropentan-2-yl)carbamate (800 mg, 1.19 mmol, 1 eq), Boc₂O (391 mg, 1.79 mmol, 1.5 eq) and DMAP (73 mg, 0.597 mmol, 0.5 eq) in DCM (14 mL) was stirred at 20° C. for 2 hours under N₂ atmosphere. After the completion of the reaction, the mixture was poured into H₂O (150 mL) and extracted with EtOAc (200 mL×2). The combined organic layers were washed with brine (150 mL), dried over anhydrous Na₂SO₄, filtered, and the filtrate was concentrated in vacuo. The resulting residue was purified by column chromatography to tert-butyl (2-(((S)-4-((tert-butoxycarbonyl)amino)-3,3-difluoropentyl)oxy)pyridin-4-yl)(1-(tert-butyl)-3-((1R,3S,4R)-3-((tert-butyldimethylsilyl)oxy)-4-fluorocyclopentyl)-1H-pyrazol-5-yl)carbamate (825 mg, 89% yield) as a yellow oil. LCMS: 770.2 [M+H]⁺.

Step 9: Preparation of tert-butyl (2-(((S)-4-((tert-butoxycarbonyl)amino)-3,3-difluoropentyl)oxy)pyridin-4-yl)(1-(tert-butyl)-3-((1R,3R,4S)-3-fluoro-4-hydroxycyclopentyl)-1H-pyrazol-5-yl)carbamate

A mixture of tert-butyl (2-(((S)-4-((tert-butoxycarbonyl)amino)-3,3-difluoropentyl)oxy)pyridin-4-yl)(1-(tert-butyl)-3-((1R,3S,4R)-3-((tert-butyldimethylsilyl)oxy)-4-fluorocyclopentyl)-1H-pyrazol-5-yl)carbamate (815 mg, 1.06 mmol, 1 eq) and TBAF (1 M, 3.7 mL, 3.5 eq) in THF (16 mL) was stirred at 20° C. for 12 hours under N₂ atmosphere. After the completion of the reaction, the mixture was poured into H₂O (150 mL) and extracted with EtOAc (150 mL×2). The combined organic layers were washed with brine (150 mL), dried over anhydrous Na₂SO₄, filtered, and the filtrate was concentrated under reduced pressure to give tert-butyl (2-(((S)-4-((tert-butoxycarbonyl)amino)-3,3-difluoropentyl)oxy)pyridin-4-yl)(1-(tert-butyl)-3-((1R,3R,4S)-3-fluoro-4-hydroxycyclopentyl)-1H-pyrazol-5-yl)carbamate (694 mg, crude) as a yellow oil, which was used in the next step without further purification. LCMS: 656.3 [M+H]⁺.

Step 10: Preparation of tert-butyl (2-(((S)-4-((tert-butoxycarbonyl)amino)-3,3-difluoropentyl)oxy)pyridin-4-yl)(1-(tert-butyl)-3-((1R,3R,4S)-3-fluoro-4-(((4-nitrophenoxy)carbonyl)oxy)cyclopentyl)-1H-pyrazol-5-yl)carbamate

A mixture of tert-butyl (2-(((S)-4-((tert-butoxycarbonyl)amino)-3,3-difluoropentyl)oxy)pyridin-4-yl)(1-(tert-butyl)-3-((1R,3R,4S)-3-fluoro-4-hydroxycyclopentyl)-1H-pyrazol-5-yl)carbamate (684 mg, 1.04 mmol, 1 eq), 4-nitrophenyl carbonochloridate (631 mg, 3.13 mmol, 3 eq), DMAP (127 mg, 1.04 mmol, 1 eq) and Pyridine (495 mg, 6.26 mmol, 6 eq) in DCM (14 mL) was stirred at 20° C. for 1 hour. After the completion of the reaction, the mixture was then poured into H₂O (100 mL), extracted with EtOAc (100 mL×2). The combined organic layers were washed with brine (100 mL), dried over anhydrous Na₂SO₄, filtered, and the filtrate was concentrated in vacuo. The resulting residue was purified by column chromatography to give tert-butyl (2-(((S)-4-((tert-butoxycarbonyl)amino)-3,3-difluoropentyl)oxy)pyridin-4-yl)(1-(tert-butyl)-3-((1R,3R,4S)-3-fluoro-4-(((4-nitrophenoxy)carbonyl)oxy)cyclopentyl)-1H-pyrazol-5-yl)carbamate (793 mg, 0.966 mmol, 93% yield) as a yellow oil. LCMS: 821.3 [M+H]⁺.

Step 11: Preparation of (1S,2R,4R)-4-(5-((2-(((S)-4-amino-3,3-difluoropentyl)oxy)pyridin-4-yl)amino)-1-(tert-butyl)-1H-pyrazol-3-yl)-2-fluorocyclopentyl (4-nitrophenyl) carbonate

A mixture of tert-butyl (2-(((S)-4-((tert-butoxycarbonyl)amino)-3,3-difluoropentyl)oxy)pyridin-4-yl)(1-(tert-butyl)-3-((1R,3R,4S)-3-fluoro-4-(((4-nitrophenoxy)carbonyl)oxy)cyclopentyl)-1H-pyrazol-5-yl)carbamate (783 mg, 0.954 mmol, 1 eq) and TFA (5 mL) in DCM (20 mL) was stirred at 20° C. for 12 hours. After the completion of the reaction, the mixture was concentrated in vacuo. The desired (1S,2R,4R)-4-(5-((2-(((S)-4-amino-3,3-difluoropentyl)oxy)pyridin-4-yl)amino)-1-(tert-butyl)-1H-pyrazol-3-yl)-2-fluorocyclopentyl (4-nitrophenyl) carbonate (592 mg, crude) was obtained as a yellow oil, which was used into the next step without further purification.

Step 12: Preparation of (1¹R,1³S,1⁴R,9S,Z)-2¹-(tert-butyl)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one

A mixture of (1S,2R,4R)-4-(5-((2-(((S)-4-amino-3,3-difluoropentyl)oxy)pyridin-4-yl)amino)-1-(tert-butyl)-1H-pyrazol-3-yl)-2-fluorocyclopentyl (4-nitrophenyl) carbonate (582 mg, 0.938 mmol, 1 eq) and DIEA (2.67 g, 20.63 mmol, 3.59 mL, 22 eq) in DMSO (300 mL) was stirred at 100° C. for 12 hours under N₂ atmosphere. After the completion of the reaction, the mixture was poured into H₂O (150 mL) and extracted with EtOAc (150 mL×2). The combined organic layers were washed with brine (150 mL), dried over anhydrous Na₂SO₄, filtered, and the filtrate was concentrated in vacuo. The resulting residue was purified by column chromatography to give (1¹R,1³S,1⁴R,9S,Z)-2¹-(tert-butyl)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (400 mg, 88% yield) as a yellow solid. LCMS: 482.2 [M+H]⁺.

Step 13: Preparation of (1¹R,1³S,1⁴R,9S,Z)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (compound 226) and (1¹S,1³R,1⁴S,9S,Z)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (compound 227)

A solution of (1¹R,1³S,1⁴R,9S,Z)-2¹-(tert-butyl)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (130 mg, 0.27 mmol, 1 eq) in HCOOH (2.5 mL) was stirred at 100° C. for 1 hour under N₂ atmosphere. After the completion of the reaction, the mixture was cooled to room temperature and concentrated in vacuo. The resulting residue was purified by prep-HPLC (column: Welch Xtimate C18 150*25 mm*5 um; mobile phase: [water(FA)-ACN]; gradient:2%-32% B over 10 min) and further by SFC (column: DAICEL CHIRALPAK IC(250 mm*30 mm, 10 um); mobile phase: [CO₂-MeOH(0.1% NH₃H₂O)]; B %:30%, isocratic elution mode) to give (11R,13S1⁴R,9S,Z)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (22.8 mg, 41% yield) as a white solid, and (1¹S,1³R,1⁴S,9S,Z)-1⁴,8,8-trifluoro-9-methyl-2¹H-5,12-dioxa-3,10-diaza-4(4,2)-pyridina-2(3,5)-pyrazola-1 (1,3)-cyclopentanacyclododecaphan-11-one (19.6 mg, 35% yield) as a white solid.

(1¹R,1³S,1⁴R,9S,Z)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one. LCM5: 426.3 [M+H]⁺; 1H NMR (400 MHz, DMSO-d₆) δ 12.22-12.07 (m, 1H), 9.06-8.92 (m, 1H), 7.76 (d, J=5.5 Hz, 1H), 7.68-7.56 (m, 1H), 6.46 (br d, J=5.6 Hz, 1H), 6.36 (s, 1H), 6.24-6.11 (m, 1H), 5.22-5.13 (m, 1H), 5.13-4.94 (m, 1H), 4.42-4.29 (m, 1H), 4.21-3.94 (m, 2H), 3.21-3.15 (m, 1H), 2.40 (br s, 2H), 2.35 (br d, J=16.5 Hz, 2H), 2.19-2.10 (m, 1H), 1.79-1.58 (m, 1H), 1.18 (br d, J=7.0 Hz, 3H) ppm.

(1¹S,1³R,1⁴S,9S,Z)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one. LCMS: 426.3 [M+H]⁺; ¹H NMR (400 MHz, DMSO-d₆) δ 11.89 (br d, J=14.5 Hz, 1H), 9.00-8.87 (m, 1H), 8.28-8.24 (m, 1H), 7.83-7.68 (m, 1H), 7.45-7.07 (m, 1H), 6.51-6.30 (m, 2H), 6.27-6.10 (m, 1H), 5.38-4.85 (m, 2H), 4.41-3.88 (m, 3H), 3.14-3.05 (m, 1H), 2.35-2.02 (m, 4H), 2.02-1.81 (m, 1H), 1.61 (s, 1H), 1.15 (d, J=6.9 Hz, 3H) ppm.

Example 54: Synthesis of (1¹R,1³S,1⁴R,9S,E)-1⁴,2⁴,8,8-tetrafluoro-9-methyl-2¹H-5,12-dioxa-3,10-diaza-4(4,2)-pyridina-2(3,5)-pyrazola-1(1,3)-cyclopentanacyclododecaphan-11-one (compound 239) and (1¹S,1³R,1⁴S,9S,E)-1⁴,2⁴,8,8-tetrafluoro-9-methyl-2¹H-5,12-dioxa-3,10-diaza-4(4,2)-pyridina-2(3,5)-pyrazola-1(1,3)-cyclopentanacyclododecaphan-11-one (compound 240)

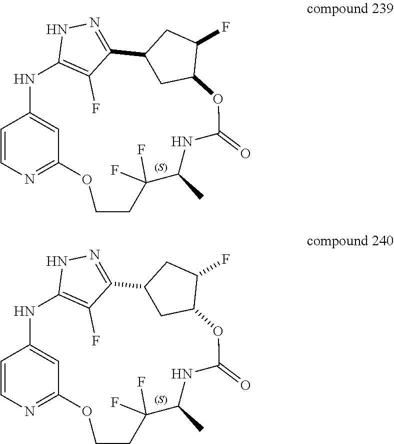

Step 1: Preparation of (11R,1³S,14R,9S,E)-21-(tert-butyl)-1⁴,2⁴,8,8-tetrafluoro-9-methyl-2¹H-5,12-dioxa-3,10-diaza-4(4,2)-pyridina-2(3,5)-pyrazola-1(1,3)-cyclopentanacyclododecaphan-11-one

To a solution of (1¹R,1⁵S,1⁴R,9S,Z)-2¹-(tert-butyl)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one (150 mg, 0.311 mmol, 1 eq) in ACN (3 mL) was added Select-F (121 mg, 0.342 mmol, 1.1 eq), and the mixture was stirred at 20° C. for 3 hours under N₂ atmosphere. After the completion of the reaction, the mixture was quenched with saturated aqueous NaHCO₃(50 mL) and extracted with EtOAc (50 mL×2). The combined organic layers were washed with brine (50 mL), dried over anhydrous Na₂SO₄, filtered, and the filtrate was concentrated in vacuo. The resulting residue was purified by prep-TLC (PE:EA=0:1) to afford (1¹R,1³S,1⁴R,9S,E)-2¹-(tert-butyl)-1⁴,2⁴,8,8-tetrafluoro-9-methyl-2¹H-5,12-dioxa-3,10-diaza-4(4,2)-pyridina-2(3,5)-pyrazola-1 (1,3)-cyclopentanacyclododecaphan-11-one (105 mg, 0.210 mmol, 67% yield) as a yellow oil. LCMS: 500.2 [M+H]⁺.

Step 2: Preparation of (1¹R,1³S,1⁴R,9S,E)-1⁴,2⁴,8,8-tetrafluoro-9-methyl-2¹H-5,12-dioxa-3,10-diaza-4(4,2)-pyridina-2(3,5)-pyrazola-1 (1,3)-cyclopentanacyclododecaphan-11-one (compound 239) and (1¹S,1³R,1⁴S,9S,E)-1⁴,2⁴,8,8-tetrafluoro-9-methyl-2¹H-5,12-dioxa-3,10-diaza-4(4,2)-pyridina-2(3,5)-pyrazola-1(1,3)-cyclopentanacyclododecaphan-11-one (compound 240)

A mixture of (1¹R,1³S,1⁴R,9S,E)-2¹-(tert-butyl)-1⁴,2⁴,8,8-tetrafluoro-9-methyl-2¹H-5,12-dioxa-3,10-diaza-4(4,2)-pyridina-2(3,5)-pyrazola-1 (1,3)-cyclopentanacyclododecaphan-11-one (105 mg, 0.210 mmol, 1 eq) in HCOOH (2 mL) was stirred at 100° C. for 1 hour under N₂ atmosphere. After the completion of the reaction, the mixture was cooled to room temperature and concentrated in vacuo. The resulting residue was purified by prep-HPLC (column: Phenomenex luna C18 150*25 mm*10 um; mobile phase: [water(FA)-ACN]; gradient:4%-34% B over 10 min) and further by SFC (column: DAICEL CHIRALPAK IK (250 mm*30 mm, 10 um); mobile phase: [CO₂-MeOH(0.1% NH₃H₂O)]; B %:40%, isocratic elution mode) to give (1¹R,1³S,1⁴R,9S,E)-1⁴,2⁴,8,8-tetrafluoro-9-methyl-2¹H-5,12-dioxa-3,10-diaza-4(4,2)-pyridina-2(3,5)-pyrazola-1(1,3)-cyclopentanacyclododecaphan-11-one (12 mg, 26% yield) as an off-white solid, and (1¹S,1³R,1⁴S,9S,E)-1⁴,2⁴,8,8-tetrafluoro-9-methyl-2¹H-5,12-dioxa-3,10-diaza-4(4,2)-pyridina-2(3,5)-pyrazola-1(1,3)-cyclopentanacyclododecaphan-11-one (23.8 mg, 52% yield) as an off-white solid.

(1¹R,1³S,1⁴R,9S,E)-1⁴,2⁴,8,8-tetrafluoro-9-methyl-2¹H-5,12-dioxa-3,10-diaza-4(4,2)-pyridina-2(3,5)-pyrazola-1(1,3)-cyclopentanacyclododecaphan-11-one. LCMS: 444.3 [M+H]⁺; ¹H NMR (400 MHz, DMSO-d₆) δ 12.49-12.22 (m, 1H), 8.85-8.75 (m, 1H), 7.78 (d, J=5.5 Hz, 1H), 7.52 (br d, J=9.5 Hz, 1H), 6.51-6.40 (m, 1H), 5.98-5.80 (m, 1H), 5.25-4.95 (m, 2H), 4.28-4.05 (m, 2H), 4.04-3.89 (m, 1H), 3.29 (br d, J=2.8 Hz, 1H), 2.47-2.38 (m, 3H), 2.35-2.23 (m, 2H), 1.96-1.82 (m, 1H), 1.13 (br d, J=6.9 Hz, 3H) ppm.

(1¹S,1³R,1⁴S,9S,E)-1⁴,2⁴,8,8-tetrafluoro-9-methyl-2¹H-5,12-dioxa-3,10-diaza-4(4,2)-pyridina-2(3,5)-pyrazola-1(1,3)-cyclopentanacyclododecaphan-11-one. LCMS: 444.3 [M+H]⁺; ¹H NMR (400 MHz, DMSO-d₆) δ 12.60-12.09 (m, 1H), 8.97-8.61 (m, 1H), 7.77 (br d, J=5.0 Hz, 1H), 7.30-6.91 (m, 1H), 6.46 (br d, J=5.4 Hz, 1H), 5.97-5.63 (m, 1H), 5.40-4.86 (m, 2H), 4.31-4.00 (m, 2H), 3.97-3.79 (m, 1H), 3.24-3.20 (m, 1H), 2.44-2.20 (m, 5H), 1.99-1.80 (m, 1H), 1.19-1.00 (m, 3H) ppm.

Example 55: Synthesis of (1¹S,1³R,8R,9S,Z)-8-fluoro-9-methyl-2¹H-5,12-dioxa-3,10-diaza-4(4,2)-pyridina-2(3,5)-pyrazola-1(1,3)-cyclopentanacyclododecaphan-11-one (compound 257) and (1¹S,1³R,8S,9S,Z)-8-fluoro-9-methyl-2¹H-5,12-dioxa-3,10-diaza-4(4,2)-pyridina-2(3,5)-pyrazola-1(1,3)-cyclopentanacyclododecaphan-11-one (compound 258)

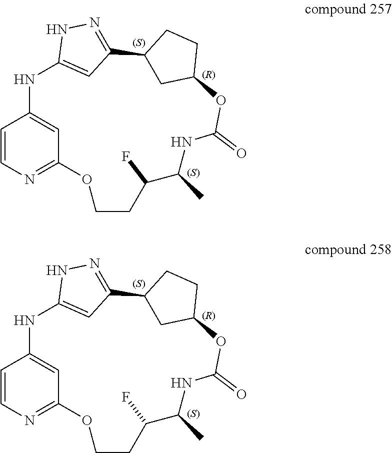

Step 1: Preparation of 3-fluorodihydrofuran-2(3H)-one

To a solution of 3-hydroxydihydrofuran-2(3H)-one (15 g, 147 mmol, 1 eq) in DCM (126 mL) was added DAST (71 g, 440.7 mmol, 3 eq) at −78° C. The mixture was then raised to 20° C. and stirred for 2 hours under N₂ atmosphere. After the completion of the reaction, the mixture was poured into water (250 mL) at 0° C. and extracted with DCM (250 mL×3). The combined organic phase was dried over Na₂SO₄, filtered, and the filtrate was concentrated. The resulting residue was purified by column chromatography to afford 3-fluorodihydrofuran-2(3H)-one (7 g, 46% yield) as a yellow oil. ¹H NMR (400 MHz, CHLOROFORM-d) δ 5.30-5.00 (m, 1H), 4.39 (td, J=8.9, 3.5 Hz, 1H), 4.23 (td, J=8.9, 6.8 Hz, 1H), 2.78-2.55 (m, 1H), 2.50-2.24 (m, 1H) ppm.

Step 2: Preparation of 2-fluoro-4-hydroxy-N-methoxy-N-methylbutanamide

To a solution of 3-fluorodihydrofuran-2(3H)-one (4.5 g, 43.24 mmol, 1 eq) and N,O-dimethylhydroxylamine HCl (10.5 g, 108 mmol, 2.5 eq) in DCM (240 mL) was added Al(CH₃)₃ (2 M, 54 mL, 2.5 eq) dropwise at 0° C. under N₂ atmosphere. The mixture was then raised to 20° C. and stirred for 1 hour under N₂ atmosphere. After the completion of the reaction, the mixture was poured into water (500 mL) and extracted with DCM (500 mL×3). The combined organic phase was dried over Na₂SO₄, filtered, and the filtrate was concentrated. The resulting residue was purified by column chromatography to give 2-fluoro-4-hydroxy-N-methoxy-N-methylbutanamide (2.3 g, 32% yield) as a colorless oil. ¹H NMR (400 MHz, CHLOROFORM-d) δ 5.53-5.25 (m, 1H), 3.83-3.61 (m, 5H), 3.17 (s, 3H), 2.88 (br s, 1H), 2.15-1.93 (m, 2H) ppm.

Step 3: Preparation of 4-((4-bromopyridin-2-yl)oxy)-2-fluoro-N-methoxy-N-methylbutanamide

To a solution of 2-fluoro-4-hydroxy-N-methoxy-N-methylbutanamide (1.3 g, 8 mmol, 1 eq), 4-bromo-2-fluoropyridine (2.13 g, 12.12 mmol, 1.5 eq) in THF (15 mL) was added t-BuOK/THF (1 M, 10.50 mL, 1.3 eq) at 0° C. The mixture was then raised to 20° C. and stirred for 2 hours under N₂ atmosphere. After the completion of the reaction, the reaction mixture was poured into water (150 mL) and extracted with DCM (150 mL×3). The combined organic phase was dried over Na₂SO₄, filtered, and the filtrate was concentrated. The resulting residue was purified by column chromatography to give 4-((4-bromopyridin-2-yl)oxy)-2-fluoro-N-methoxy-N-methylbutanamide (1.1 g, 42% yield) as a yellow oil. LCMS: 321.1; 323.1 [M+H]⁺.

Step 4: Preparation of 5-((4-bromopyridin-2-yl)oxy)-3-fluoropentan-2-one

To a solution of 4-((4-bromopyridin-2-yl)oxy)-2-fluoro-N-methoxy-N-methylbutanamide (900 mg, 2.8 mmol, 1 eq) in THF (10 mL) was added MeMgBr (3 M, 2.25 mL, 2.4 eq) at 0° C. The mixture was then raised to 20° C. and stirred for 3 hours under N₂ atmosphere. After the completion of the reaction, the mixture was quenched with H₂O (100 mL) and extracted with DCM (100 mL×3). The combined organic layers were washed with brine (100 mL), dried over anhydrous Na₂SO₄, filtered and the filtrate was concentrated in vacuo. The resulting residue was purified by column chromatography to afford 5-((4-bromopyridin-2-yl)oxy)-3-fluoropentan-2-one (700 mg, 90% yield) as a yellow oil. LCMS: 276.0; 278.0 [M+H]⁺.

Step 5: Preparation of (R)—N-((2S)-5-((4-bromopyridin-2-yl)oxy)-3-fluoropentan-2-yl)-2-methylpropane-2-sulfinamide

To a solution of 5-((4-bromopyridin-2-yl)oxy)-3-fluoropentan-2-one (0.6 g, 2.17 mmol, 1 eq) and (S)-2-methylpropane-2-sulfinamide (526 mg, 4.35 mmol, 2.0 eq) in THF (8 mL) was added tetraisopropoxytitanium (2.47 g, 8.69 mmol, 4 eq), and the mixture was stirred at 80° C. for 16 hours under N₂ atmosphere. The mixture was then cooled to 20° C., and the NaBH₄ (246.64 mg, 6.52 mmol, 3 eq) was added in portions. After the completion of the addition, the reaction mixture was kept stirring at 20° C. for 2 hours. The mixture was subsequently quenched with H₂O (100 mL) and extracted with DCM (100 mL×3). The combined organic layers were washed with brine (100 mL), dried over anhydrous Na₂SO₄, filtered and the filtrate was concentrated in vacuo. The resulting residue was purified by column chromatography to give (R)—N-((2S)-5-((4-bromopyridin-2-yl)oxy)-3-fluoropentan-2-yl)-2-methylpropane-2-sulfinamide (800 mg, 96% yield) as a yellow oil. LCMS: 381.1; 383.1 [M+H]⁺.

Step 6: Preparation of (2S)-5-((4-bromopyridin-2-yl)oxy)-3-fluoropentan-2-amine

To a solution of (R)—N-((2S)-5-((4-bromopyridin-2-yl)oxy)-3-fluoropentan-2-yl)-2-methylpropane-2-sulfinamide (600 mg, 1.57 mmol, 1 eq) in dioxane (1 mL) was added HCl/dioxane (2 M, 13 mL, 16 eq), and the mixture was stirred at 20° C. for 3 hours. After the completion of the reaction, the reaction mixture was poured into aqueous NaHCO₃ solution (50 mL) and extracted with DCM (50 mL×3). The combined organic layers were washed with brine (100 mL), dried over anhydrous Na₂SO₄, filtered and the filtrate was concentrated in vacuo. The resulting residue was purified by column chromatography to give (2S)-5-((4-bromopyridin-2-yl)oxy)-3-fluoropentan-2-amine (430 mg, 1.55 mmol, 98% yield) as a white solid.

Step 7: Preparation of tert-butyl ((2S)-5-((4-bromopyridin-2-yl)oxy)-3-fluoropentan-2-yl)carbamate

To a solution of (2S)-5-((4-bromopyridin-2-yl)oxy)-3-fluoropentan-2-amine (430 mg, 1.55 mmol, 1 eq) in DCM (5 mL) was added Boc₂O (1.02 g, 4.65 mmol, 3 eq) and TEA (314 mg, 3.1 mmol, 2 eq), and the mixture was stirred at 20° C. for 16 hours. After the completion of the reaction, the reaction mixture was poured into H₂O (50 mL) and extracted with DCM (50 mL×3). The combined organic layers were washed with brine (100 mL), dried over anhydrous Na₂SO₄, filtered and the filtrate was concentrated in vacuo. The resulting residue was purified by column chromatography to give tert-butyl ((2S)-5-((4-bromopyridin-2-yl)oxy)-3-fluoropentan-2-yl)carbamate (580 mg, 1.54 mmol, 99% yield) as a yellow oil. LCMS: 377.1; 379.1 [M+H]⁺.

Step 8: Preparation of tert-butyl ((2S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-3-fluoropentan-2-yl)carbamate

A mixture of tert-butyl ((2S)-5-((4-bromopyridin-2-yl)oxy)-3-fluoropentan-2-yl)carbamate (544 mg, 1.44 mmol, 1 eq), 1-(tert-butyl)-3-((1S,3R)-3-((tert-butyldimethylsilyl)oxy)cyclopentyl)-1H-pyrazol-5-amine (536 mg, 1.59 mmol, 1.1 eq), Xantphos (167 mg, 0.288 mmol, 0.2 eq), Pd₂(dba)₃ (132 mg, 0.144 mmol, 0.1 eq) and Cs₂CO₃ (1.41 g, 4.33 mmol, 3 eq) in dioxane (10 mL) was degassed and backfilled with N₂ for 3 times, and then stirred at 100° C. for 16 hours under N₂ atmosphere. After the completion of the reaction, the mixture was cooled to room temperature. The mixture was then poured into H₂O (100 mL) and extracted with DCM (100 mL×3). The combined organic layers were washed with brine (100 mL), dried over anhydrous Na₂SO₄, filtered, and the filtrate was concentrated in vacuo. The resulting residue was purified by column chromatography to give tert-butyl ((2S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-3-fluoropentan-2-yl)carbamate (900 mg, 98% yield) as a yellow oil. LCMS: 634.4 [M+H]⁺.

Step 9: Preparation of tert-butyl (2-(((4S)-4-((tert-butoxycarbonyl)amino)-3-fluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate

To a solution of tert-butyl ((2S)-5-((4-((1-(tert-butyl)-3-((1S,3R)-3-((tert-butyldimethylsilyl)oxy)cyclopentyl)-1H-pyrazol-5-yl)amino)pyridin-2-yl)oxy)-3-fluoropentan-2-yl)carbamate (550 mg, 0.867 mmol, 1 eq) in DCM (7 mL) was added Boc₂O (284 mg, 1.30 mmol, 1.5 eq) and DMAP (53 mg, 0.433 mmol, 0.5 eq), and the mixture was stirred at 20° C. for 3 hours. After the completion of the reaction, the reaction mixture was poured into H₂O (100 mL) and extracted with DCM (100 mL×3). The combined organic layers were washed with brine (100 mL), dried over anhydrous Na₂SO₄, filtered, and the filtrate was concentrated in vacuo. The resulting residue was purified by column chromatography to give tert-butyl (2-(((4S)-4-((tert-butoxycarbonyl)amino)-3-fluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate (630 mg, 98% yield) as a yellow oil. LCMS: 734.4 [M+H]⁺.

Step 10: Preparation of tert-butyl (2-(((4S)-4-((tert-butoxycarbonyl)amino)-3-fluoropentyl)oxy)pyridin-4-yl)(1-(tert-butyl)-3-((1S,3R)-3-hydroxycyclopentyl)-1H-pyrazol-5-yl)carbamate

A mixture of tert-butyl (2-(((4S)-4-((tert-butoxycarbonyl)amino)-3-fluoropentyl)oxy)pyridin-4-yl)(1-(tert-butyl)-3-((1S,3R)-3-((tert-butyldimethylsilyl)oxy)cyclopentyl)-1H-pyrazol-5-yl)carbamate (600 mg, 0.817 mmol, 1 eq) and TBAF (2.14 g, 8.17 mmol, 10 eq) in THF (5 mL) was stirred at 20° C. for 2 hours. After the completion of the reaction, the reaction mixture was poured into H₂O (100 mL) and extracted with DCM (100 mL×3). The combined organic layers were washed with brine (100 mL), dried over anhydrous Na₂SO₄, filtered, and the filtrate was concentrated in vacuo. The desired tert-butyl (2-(((4S)-4-((tert-butoxycarbonyl)amino)-3-fluoropentyl)oxy)pyridin-4-yl)(1-(tert-butyl)-3-((1S,3R)-3-hydroxycyclopentyl)-1H-pyrazol-5-yl)carbamate (500 mg, crude) was obtained as a yellow oil, which was used into the next step without further purification.

Step 11: Preparation of tert-butyl (3R)-6-((4-((tert-butoxycarbonyl)(1-(tert-butyl)-3-((1S,3R)-3-(((4-nitrophenoxy)carbonyl)oxy)cyclopentyl)-1H-pyrazol-5-yl)amino)pyridin-2-yl)oxy)-4-fluoro-3-methylhexanoate

To a mixture of tert-butyl (2-(((4S)-4-((tert-butoxycarbonyl)amino)-3-fluoropentyl)oxy)pyridin-4-yl)(1-(tert-butyl)-3-((1S,3R)-3-hydroxycyclopentyl)-1H-pyrazol-5-yl)carbamate (500 mg, 0.806 mmol, 1 eq) in DCM (8 mL) was added 4-nitrophenyl carbonochloridate (406 mg, 2.02 mmol, 2.5 eq), DMAP (98 mg, 0.806 mmol, 1 eq) and Pyridine (319 mg, 4.03 mmol, 5 eq), and the mixture was stirred at 20° C. for 2 hours. After the completion of the reaction, the reaction mixture was poured into H₂O (100 mL) and extracted with DCM (100 mL×3). The combined organic layers were washed with brine (100 mL), dried over anhydrous Na₂SO₄, filtered, and the filtrate was concentrated in vacuo. The resulting residue was purified by column chromatography to give tert-butyl (3R)-6-((4-((tert-butoxycarbonyl)(1-(tert-butyl)-3-((1S,3R)-3-(((4-nitrophenoxy)carbonyl)oxy)cyclopentyl)-1H-pyrazol-5-yl)amino)pyridin-2-yl)oxy)-4-fluoro-3-methylhexanoate (630 mg, 98% yield) as a yellow oil. LCMS: 785.4 [M+H]⁺.

Step 12: Preparation of (1R,3S)-3-(5-((2-(((4S)-4-amino-3-fluoropentyl)oxy)pyridin-4-yl)amino)-1-(tert-butyl)-1H-pyrazol-3-yl)cyclopentyl (4-nitrophenyl) carbonate

A mixture of tert-butyl (3R)-6-((4-((tert-butoxycarbonyl)(1-(tert-butyl)-3-((1S,3R)-3-(((4-nitrophenoxy)carbonyl)oxy)cyclopentyl)-1H-pyrazol-5-yl)amino)pyridin-2-yl)oxy)-4-fluoro-3-methylhexanoate (600 mg, 0.764 mmol, 1 eq) and TFA (6 mL) in DCM (6 mL) was stirred at 20° C. for 3 hours. After the completion of the reaction, the mixture was concentrated in vacuo. The desired (1R,3S)-3-(5-((2-(((4S)-4-amino-3-fluoropentyl)oxy)pyridin-4-yl)amino)-1-(tert-butyl)-1H-pyrazol-3-yl)cyclopentyl (4-nitrophenyl) carbonate (440 mg, crude) was obtained as a yellow oil, which was used into the next step without further purification.

Step 13: Preparation of (1¹S,1³R,9S,Z)-2¹-(tert-butyl)-8-fluoro-9-methyl-2¹H-5,12-dioxa-3,10-diaza-4(4,2)-pyridina-2(3,5)-pyrazola-1(1,3)-cyclopentanacyclododecaphan-11-one

A mixture of (1R,3S)-3-(5-((2-(((4S)-4-amino-3-fluoropentyl)oxy)pyridin-4-yl)amino)-1-(tert-butyl)-1H-pyrazol-3-yl)cyclopentyl (4-nitrophenyl) carbonate (200 mg, 0.342 mmol, 1 eq) and DIEA (88 mg, 0.684 mmol, 2 eq) in DMSO (200 mL) was stirred at 100° C. for 2 hours under N₂ atmosphere. After the completion of the reaction, the reaction mixture was cooled to room temperature, poured into H₂O (400 mL) and extracted with DCM (200 mL×3). The combined organic layers were washed with brine (200 mL), dried over anhydrous Na₂SO₄, filtered, and the filtrate was concentrated in vacuo. The resulting residue was purified by column chromatography to give (1¹S,1³R,9S,Z)-2¹-(tert-butyl)-8-fluoro-9-methyl-2¹H-5,12-dioxa-3,10-diaza-4(4,2)-pyridina-2(3,5)-pyrazola-1(1,3)-cyclopentanacyclododecaphan-11-one (150 mg, 0.336 mmol, 97% yield) as a yellow oil. LCMS: 446.2 [M+H]⁺.

Step 14: Preparation of (1¹S,1³R,8R,9S,Z)-8-fluoro-9-methyl-2¹H-5,12-dioxa-3,10-diaza-4(4,2)-pyridina-2(3,5)-pyrazola-1(1,3)-cyclopentanacyclododecaphan-11-one (compound 257) and (1¹S,1³R,8S,9S,Z)-8-fluoro-9-methyl-2¹H-5,12-dioxa-3,10-diaza-4(4,2)-pyridina-2(3,5)-pyrazola-1(1,3)-cyclopentanacyclododecaphan-11-one (compound 258)

A mixture of (1¹S,1³R,9S,Z)-2¹-(tert-butyl)-8-fluoro-9-methyl-2¹H-5,12-dioxa-3,10-diaza-4(4,2)-pyridina-2(3,5)-pyrazola-1(1,3)-cyclopentanacyclododecaphan-11-one (150 mg, 0.336 mmol, 1 eq) and formic acid (1 mL) was stirred at 100° C. for 1 hour under N₂ atmosphere. After the completion of the reaction, the mixture was cooled to room temperature, and then concentrated in vacuo. The resulting residue was purified by SFC (condition: column: DAICEL CHIRALCEL OX (250 mm*30 mm, 10 um); mobile phase: [CO₂-EtOH(0.1% NH₃H₂O)]; B %:30%, isocratic elution mode) to give (1¹S,1³R,8R,9S,Z)-8-fluoro-9-methyl-2¹H-5,12-dioxa-3,10-diaza-4(4,2)-pyridina-2(3,5)-pyrazola-1(1,3)-cyclopentanacyclododecaphan-11-one (15.45 mg, 11.6% yield) as an off-white solid and (1¹S,1³R,8S,9S,Z)-8-fluoro-9-methyl-2¹H-5,12-dioxa-3,10-diaza-4(4,2)-pyridina-2(3,5)-pyrazola-1(1,3)-cyclopentanacyclododecaphan-11-one (24.64 mg, 0.063 mmol, 18.7% yield) as a yellow solid.

(1¹S,1³R,8R,9S,Z)-8-fluoro-9-methyl-2¹H-5,12-dioxa-3,10-diaza-4(4,2)-pyridina-2(3,5)-pyrazola-1(1,3)-cyclopentanacyclododecaphan-11-one. LCMS: 390.2 [M+H]⁺; ¹H NMR (400 MHz, DMSO-d₆, 22° C.) δ 12.14 (br s, 1H), 9.06-8.80 (m, 1H), 7.76 (d, J=5.7 Hz, 1H), 7.23 (br d, J=9.3 Hz, 1H), 6.43 (dd, J=5.7, 1.4 Hz, 1H), 6.28 (s, 1H), 5.94 (s, 1H), 5.00 (br s, 1H), 4.56-4.29 (m, 1H), 4.24-4.07 (m, 2H), 3.90-3.69 (m, 1H), 3.25-3.07 (m, 1H), 2.47-2.36 (m, 1H), 2.22-1.99 (m, 2H), 1.98-1.88 (m, 1H), 1.84-1.65 (m, 4H), 1.09 (br d, J=6.4 Hz, 3H) ppm.

(1¹S,1³R,8S,9S,Z)-8-fluoro-9-methyl-2¹H-5,12-dioxa-3,10-diaza-4(4,2)-pyridina-2(3,5)-pyrazola-1(1,3)-cyclopentanacyclododecaphan-11-one. LCMS: 390.3 [M+H]⁺; ¹H NMR (400 MHz, DMSO-d₆, 22° C.) δ 12.34-11.88 (m, 1H), 9.06-8.81 (m, 1H), 7.77 (d, J=5.5 Hz, 1H), 7.34-7.16 (m, 1H), 6.45 (dd, J=5.6, 1.4 Hz, 1H), 6.29 (s, 1H), 5.98 (s, 1H), 5.14-4.93 (m, 1H), 4.74-4.36 (m, 2H), 4.17-3.77 (m, 2H), 3.27-3.03 (m, 1H), 2.46-2.35 (m, 1H), 2.10-1.92 (m, 2H), 1.84-1.65 (m, 5H), 1.09-0.94 (m, 3H) ppm.

The compounds in Table 4 were prepared analogously according to the procedures described for Examples 1 to 55.

TABLE 4
Additional Compounds Prepared According to Procedures Described for
Examples 1 to 55.

| Compound | | |
|---|---|---|
| No. | Chemical Structure | Analytical Data |
| 1 | 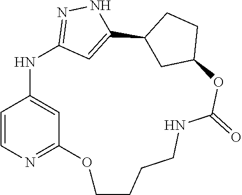 [SMILES: O=C1NCCCCOc2cc(ccn2)Nc2cc([nH]n2)[C@H]2CC[C@H](C2)O1] | LCMS: 358.2 [M + H]⁺; ¹H NMR (400 MHz, DMSO-d₆) δ 12.07 (s, 1H), 8.94 (s, 1H), 7.74 (d, J = 6.0 Hz, 1H), 7.00 (s, 1H), 6.43 (d, J = 5.6 Hz, 1H), 6.34 (s, 1H), 6.03 (s, 1H), 5.12-5.00 (m, 1H), 4.08-4.05 (m, 2H), 3.25-2.81 (m, 4H), 2.02 (m, 1H), 1.93-1.67 (m, 6H), 1.61-1.48 (m, 2H) ppm |
| 2 | 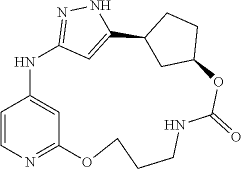 [SMILES: O=C1NCCCOc2cc(ccn2)Nc2cc([nH]n2)[C@H]2CC[C@H](C2)O1] | LCMS: 344.2 [M + H]⁺; ¹H NMR (400 MHz, DMSO-d₆) δ 12.23 (s, 1H), 8.97 (s, 1H), 8.13 (s, 1H), 7.74 (d, J = 5.6 Hz, 1H), 7.00-6.94 (m, 1H), 6.44 (dd, J = 1.5, 5.6 Hz, 1H), 6.35 (s, 1H), 5.99 (s, 1H), 4.94 (br s, 1H), 4.19-4.04 (m, 2H), 3.24 (br d, J = 6.6 Hz, 1H), 2.97-2.85 (m, 1H), 2.26 (ddd, J = 4.4, 10.8, 15.1 Hz, 1H), 2.19-2.11 (m, 1H), 1.94 (br d, J = 13.8 Hz, 1H), 1.89-1.63 (m, 6H) ppm. |
| 3 | 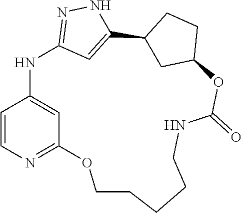 [SMILES: O=C1NCCCCCOc2cc(ccn2)Nc2cc([nH]n2)[C@H]2CC[C@H](C2)O1] | LCMS: 372.2 [M + H]⁺; ¹H NMR (400 MHz, DMSO-d₆) δ 11.97 (s, 1H), 8.83 (s, 1H), 8.16 (s, 1H), 7.72 (d, J = 5.8 Hz, 1H), 6.94 (dd, J = 4.4, 7.4 Hz, 1H), 6.41 (dd, J = 2.1, 5.9 Hz, 1H), 6.21 (d, J = 1.6 Hz, 1H), 5.95 (s, 1H), 5.01-4.90 (m, 1H), 4.40-4.26 (m, 1H), 4.14-4.00 (m, 1H), 3.24-3.12 (m, 1H), 2.85-2.70 (m, 1H), 2.45- 2.36 (m, 2H), 2.12-1.98 (m, 1H), 1.85-1.70 (m, 4H), 1.66-1.53 (m, 2H), 1.49-1.31 (m, 4H) ppm. |
| 8 | 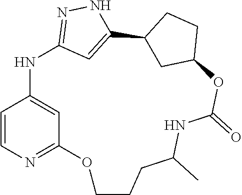 [SMILES: CC1CCCOc2cc(ccn2)Nc2cc([nH]n2)[C@H]2CC[C@H](C2)OC(=O)N1] | LCMS: 372.2 [M + H]⁺; ¹H NMR (400 MHz, DMSO-d₆) δ 12.02 (s, 1H), 8.64 (s, 1H), 8.17 (s, 1H), 7.74 (d, J = 5.8 Hz, 1H), 6.47-6.37 (m, 2H), 6.34 (s, 1H), 6.11-5.98 (m, 1H), 5.19-4.99 (m, 1H), 4.21-3.97 (m, 2H), 3.71- 3.49 (m, 1H), 3.28-3.20 (m, 1H), 2.44-2.33 (m, 1H), 2.07-1.95 (m, 1H), 1.88-1.66 (m, 6H), 1.65-1.45 (m, 2H), 1.12-0.96 (m, 3H) ppm. |
| 19 | 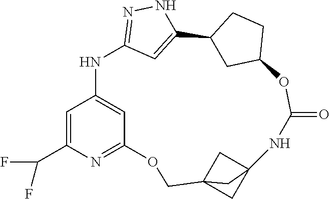 [SMILES: O=C1NC23CC(COc4cc(cc(C(F)F)n4)Nc4cc([nH]n4)[C@H]4CC[C@H](C4)O1)(C2)C3] | LCMS: 432.2 [M + H]⁺; ¹H NMR (400 MHz, DMSO-d₆) δ 12.33 (s, 1H), 8.89 (s, 1H), 7.52 (s, 1H), 6.46-6.80 (m, 2H), 6.20 (s, 1H), 5.98 (s, 1H), 5.13 (s, 1H), 4.20-4.12 (m, 2H), 3.32- 3.19 (m, 2H), 2.48-2.39 (m, 1H), 2.16-2.08 (m, 1H), 1.88-1.81 (m, 8H), 1.79-1.67 (m, 2H) ppm. |
| 64 | 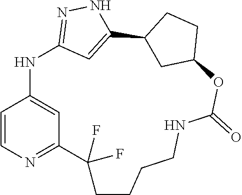 [SMILES: O=C1NCCCCC(F)(F)c2cc(ccn2)Nc2cc([nH]n2)[C@H]2CC[C@H](C2)O1] | LCMS: 392.2 [M + H]⁺; ¹H NMR (400 MHz, DMSO-d₆) δ 13.07-11.57 (m, 1 H), 10.30-9.61 (m, 1 H), 8.26 (br d, J = 6.00 Hz, 1 H), 7.32-6.69 (m, 3 H), 6.05 (s, 1 H), 5.01 (br s, 1 H), 3.33-2.83 (m, 3 H), 2.78-2.64 (m, 1 H), 2.44-2.14 (m, 3 H), 2.12-1.96 (m, 1 H), 1.94-1.64 (m, 4 H), 1.58- 1.09 (m, 5 H) ppm. |
| 96 | 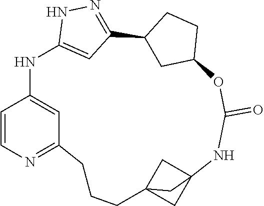 [SMILES: O=C1NC23CC(CCCc4cc(ccn4)Nc4cc(n[nH]4)[C@H]4CC[C@H](C4)O1)(C2)C3] | LCMS: 394.3 [M + H]⁺; ¹H NMR (400 MHz, DMSO-d₆) δ 12.25-12.11 (m, 1 H), 8.85 (s, 1 H), 8.01 (d, J = 5.74 Hz, 1 H), 7.50 (s, 1 H), 6.74 (d, J = 1.75 Hz, 1 H), 6.58 (dd, J = 5.62, 2.12 Hz, 1 H), 6.04 (s, 1 H), 5.14- 5.06 (m, 1 H), 3.35-3.29 (m, 6 H), 3.17-3.07 (m, 1 H), 2.09-1.99 (m, 1 H), 1.84 (s, 7 H), 1.61 (br s, 2 H), 1.37 (br d, J = 5.00 Hz, 2 H) ppm. |
| 98 | 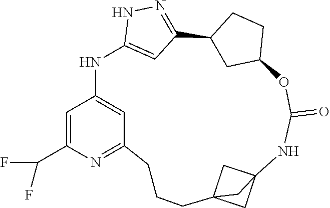 [SMILES: O=C1NC23CC(CCCc4cc(cc(C(F)F)n4)Nc4cc(n[nH]4)[C@H]4CC[C@H](C4)O1)(C2)C3] | LCMS: 444.4 [M + H]⁺; ¹H NMR (400 MHz, DMSO-d₆) δ 12.29 (br s, 1 H), 9.20 (s, 1 H), 7.52 (s, 1 H), 6.92- 6.57 (m, 3 H), 6.09 (s, 1 H), 5.12 (br d, J = 5.63 Hz, 1 H), 3.20-3.09 (m, 1 H), 2.65-2.54 (m, 3 H), 2.10-2.02 (m, 1 H), 1.89-1.62 (m, 13 H), 1.41- 1.33 (m, 2 H) ppm. |
| 100 | 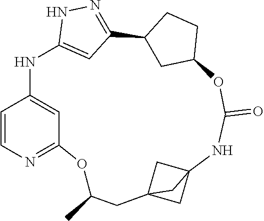 [SMILES: C[C@@H]1CC23CC(C2)(C3)NC(=O)O[C@@H]2CC[C@@H](C2)c2cc([nH]n2)Nc2ccnc(c2)O1] | LCMS: 410.3 [M + H]⁺; ¹H NMR (400 MHz, DMSO-d₆) δ 8.67 (s, 1H), 8.29- 8.27 (m, 1H), 7.76-7.75 (m, 1H), 7.37 (s, 1H), 6.43-6.41 (m, 1H), 6.14 (s, 1H), 5.91-5.80 (m, 1H), 5.10-5.09 (m, 1H), 4.73-4.68 (m, 1H), 3.15-3.10 (m, 1H), 2.07-2.06 (m, 1H), 2.04-2.03 (m, 1H), 1.91- 1.88 (m, 10H), 1.86-1.85 (m, 1H), 1.33-1.31 (m, 3H) ppm. |
| 102 | 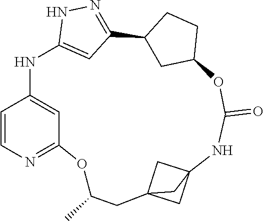 [SMILES: C[C@H]1CC23CC(C2)(C3)NC(=O)O[C@@H]2CC[C@@H](C2)c2cc([nH]n2)Nc2ccnc(c2)O1] | LCMS: 410.3 [M + H]⁺; ¹H NMR (400 MHz, DMSO-d₆) δ 12.10 (br, 1H), 8.81 (s, 1H), 7.76-7.75 (m, 1H), 7.36 (m, 1H), 6.45-6.44 (m, 1H), 6.21-6.10 (m, 1H), 6.01-5.90 (m, 1H), 5.07-4.95 (m, 1H), 4.65-4.61 (m, 1H), 3.22-3.20 (m, 1H), 2.01- 1.99 (m, 1H), 1.98-1.96 (m, 12H), 1.31-1.29 (m, 3H) ppm. |
| 106 | 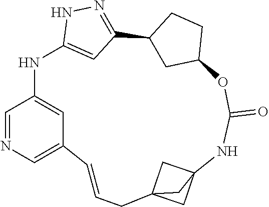 [SMILES: O=C1NC23CC(C/C=C/c4cncc(c4)Nc4cc(n[nH]4)[C@H]4CC[C@H](C4)O1)(C2)C3] | LCMS: 392.2 [M + H]⁺; ¹H NMR (400 MHz, DMSO-d₆) δ 11.82 (s, 1H), 8.57 (s, 1H), 8.18 (s, 1H), 8.08 (d, J = 2.4 Hz, 1H), 7.75 (d, J = 1.2 Hz, 1H), 7.55 (s, 2H), 6.49 (d, J = 15.6 Hz, 1H), 6.05-5.99 (m, 2H), 5.13-5.11 (m, 1H), 3.03-2.98 (m, 1H), 2.63- 2.58 (m, 1H), 2.41-2.39 (m, 2H), 2.03-1.98 (m, 1H), 1.82 (s, 6H), 1.78-1.74 (m, 2H), 1.73-1.67 (m, 2H) ppm. |
| 107 | 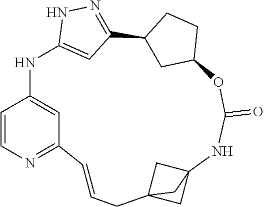 [SMILES: O=C1NC23CC(C/C=C/c4cc(ccn4)Nc4cc(n[nH]4)[C@H]4CC[C@H](C4)O1)(C2)C3] | LCMS: 392.2 [M + H]⁺; ¹H NMR (400 MHz, DMSO-d₆) δ 8.95 (br s, 1 H), 8.15 (s, 1 H), 8.03 (d, J = 5.75 Hz, 1 H), 7.54 (s, 1 H), 7.19 (d, J = 2.25 Hz, 1 H), 6.63 (dd, J = 5.74, 2.12 Hz, 1 H), 6.40 (d, J = 16.00 Hz, 1 H), 6.10-5.99 (m, 2 H), 5.12 (br s, 1 H), 3.09-2.93 (m, 2 H), 2.71-2.55 (m, 2 H), 2.40 (br d, J = 6.75 Hz, 2 H), 2.08-1.97 (m, 1 H), 1.82 (s, 6 H) 1.76-1.69 (m, 2 H) ppm. |
| 116 | 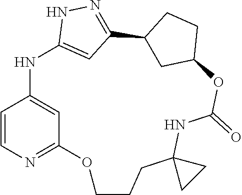 [SMILES: O=C1NC2(CCCOc3cc(ccn3)Nc3cc(n[nH]3)[C@H]3CC[C@H](C3)O1)CC2] | LCMS: 382.3 [M + H]⁺; ¹H NMR (400 MHz, DMSO-d₆) δ 8.92-8.81 (m, 1 H), 8.16 (s, 1 H), 8.01 (d, J = 5.87 Hz, 1 H), 7.46-7.35 (m, 1 H), 6.81-6.73 (m, 1 H), 6.60 (br d, J = 4.28 Hz, 1 H), 5.99 (s, 1 H), 4.96 (br d, J = 1.96 Hz, 1 H), 3.20 (br d, J = 6.85 Hz, 2 H), 2.10- 1.97 (m, 2 H), 1.83-1.61 (m, 7 H), 1.52-1.38 (m, 1 H), 1.34-1.21 (m, 1 H), 0.97-0.85 (m, 1 H), 0.69-0.42 (m, 4 H) ppm. |
| 117 | 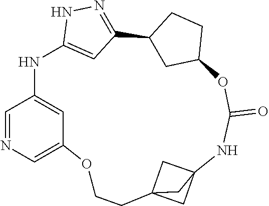 [SMILES: O=C1NC23CC(CCOc4cncc(c4)Nc4cc(n[nH]4)[C@H]4CC[C@H](C4)O1)(C2)C3] | LCMS: 396.2 [M + H]⁺; ¹H NMR (400 MHz, DMSO-d₆) δ 11.64-10.89 (m, 1H), 7.68 (s, 1H), 7.01 (d, J = 2.1 Hz, 1H), 6.82 (d, J = 2.4 Hz, 1H), 6.64 (s, 1H), 6.11 (t, J = 2.2 Hz, 1H), 5.14 (s, 1H), 4.28 (br d, J = 4.8 Hz, 1H), 3.22 (dt, J = 2.6, 6.3 Hz, 2H), 2.32-2.25 (m, 1H), 1.02-0.90 (m, 14H) ppm. |
| 118 | 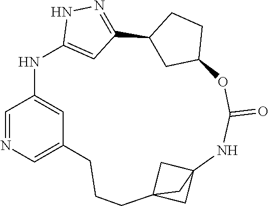 [SMILES: O=C1NC23CC(CCCc4cncc(c4)Nc4cc(n[nH]4)[C@H]4CC[C@H](C4)O1)(C2)C3] | LCMS: 394.3 [M + H]⁺; ¹H NMR (400 MHz, DMSO-d₆) δ 12.61-11.56 (m, 1H), 8.49 (s, 1H), 8.03 (s, 1H), 7.73 (s, 1H), 7.49 (s, 1H), 7.17 (s, 1H), 5.97 (s, 1H), 5.11 (d, J = 5.2 Hz, 1H), 3.17-3.00 (m, 2H), 2.61-2.52 (m, 2H), 2.07-1.99 (m, 1H), 1.84 (s, 6H), 1.81-1.64 (m, 4H), 1.64-1.54 (m, 2H), 1.42 (d, J = 5.6 Hz, 2H) ppm. |
| 119 | 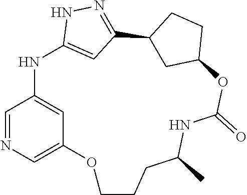 [SMILES: C[C@H]1CCCOc2cncc(c2)Nc2cc(n[nH]2)[C@H]2CC[C@H](C2)OC(=O)N1] | LCMS: 372.3 [M + H]⁺; ¹H NMR (400 MHz, DMSO-d₆) δ 12.39-11.64 (m, 1 H), 8.49 (s, 1 H), 8.20 (s, 1 H), 7.76 (d, J = 2.13 Hz, 1 H), 7.56 (d, J = 2.25 Hz, 1 H), 7.01-6.94 (m, 2 H), 5.89 (s, 1 H), 5.00 (br t, J = 4.31 Hz, 1 H), 4.18-4.08 (m, 1 H), 4.00-3.90 (m, 1 H), 3.65 (dt, J = 8.38, 6.25 Hz, 1 H), 3.19-3.13 (m, 1 H), 2.44-2.38 (m, 1 H), 2.05 (br dd, J = 12.82, 8.07 Hz, 1 H), 1.83-1.67 (m, 6 H), 1.57-1.41 (m, 2 H), 1.03 (d, J = 6.75 Hz, 3 H) ppm. |
| 120 | 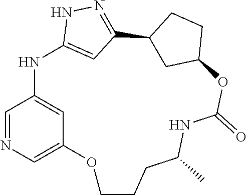 [SMILES: C[C@@H]1CCCOc2cncc(c2)Nc2cc(n[nH]2)[C@H]2CC[C@H](C2)OC(=O)N1] | LCMS: 372.2 [M + H]⁺; ¹H NMR (400 MHz, DMSO-d₆) δ 12.24-11.83 (m, 1H), 8.62-8.43 (m, 1H), 7.75 (br s, 1H), 7.52 (d, J = 2.1 Hz, 1H), 7.26- 7.13 (m, 1H), 7.01-6.84 (m, 1H), 5.89 (s, 1H), 5.22-4.94 (m, 1H), 4.48-4.32 (m, 1H), 3.25-3.06 (m, 2H), 2.73-2.64 (m, 1H), 2.42-2.32 (m, 1H), 2.07-1.97 (m, 1H), 1.82- 1.69 (m, 1H), 1.74 (br s, 1H), 1.64- 1.54 (m, 1H), 1.48-1.34 (m, 2H), 1.27-1.19 (m, 3H) ppm. |
| 121 | 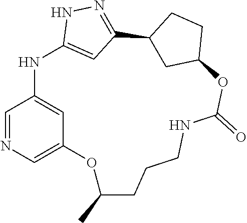 [SMILES: C[C@@H]1CCCNC(=O)O[C@@H]2CC[C@@H](C2)c2cc([nH]n2)Nc2cncc(c2)O1] | LCMS: 372.4 [M + H]⁺; ¹H NMR (400 MHz, DMSO-d₆) δ 12.20-12.03 (m, 1 H), 8.66-8.44 (m, 1 H), 8.13 (s, 1 H), 7.83-7.72 (m, 1 H), 7.63-7.48 (m, 1 H), 7.15-6.79 (m, 2 H), 5.95- 5.84 (m, 1 H), 5.00-4.86 (m, 1 H), 4.44-4.31 (m, 1 H), 3.23-3.15 (m, 1 H), 3.13-2.89 (m, 2 H), 2.88-2.60 (m, 1 H), 2.40-2.21 (m, 1 H), 2.13- 2.02 (m, 1 H), 2.00-1.84 (m, 2 H), 1.82-1.68 (m, 4 H), 1.56-1.43 (m, 2 H), 1.26 (br d, J = 4.88 Hz, 3 H) ppm. |
| 122 | 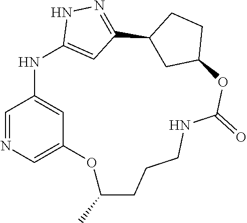 [SMILES: C[C@H]1CCCNC(=O)O[C@@H]2CC[C@@H](C2)c2cc([nH]n2)Nc2cncc(c2)O1] | LCMS: 370.1 [M + H]⁺; ¹H NMR (400 MHz, DMSO-d₆) δ 12.07 (br, 1H), 8.97 (s, 1H), 7.75 (d, J = 6.0 Hz, 1H), 7.04 (d, J = 9.0 Hz, 1H), 6.48-6.43 (m, 1H), 6.05 (s, 1H), 5.13 (s, 1H), 5.13-5.10 (m, 1H), 4.58-4.55 (m, 1H), 4.31-4.21 (m, 2H), 3.22 (s, 1H), 2.13-2.09 (m, 1H), 2.05-1.96 (m, 3H), 1.95-1.90 (m, 3H), 1.74-1.68 (m, 3H) ppm. |
| 123 | 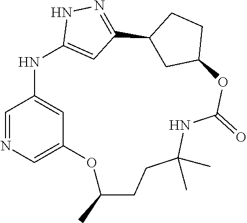 [SMILES: C[C@@H]1CCC(C)(C)NC(=O)O[C@@H]2CC[C@@H](C2)c2cc([nH]n2)Nc2cncc(c2)O1] | LCMS: 400.2 [M + H]⁺; ¹H NMR (400 MHz, DMSO-d₆) δ 11.99-11.96 (m, 1 H), 8.54-8.52 (m, 1 H), 7.76-7.75 (m, 1 H), 7.49-7.48 (m, 1 H), 6.95 (s, 1 H), 6.77-6.75 (m, 1 H), 5.93 (s, 1 H), 4.94-4.93 (m, 1 H), 4.42-4.39 (m, 1 H), 3.16 (s, 1 H), 1.99-1.80 (m, 2 H), 1.78-1.77 (m, 5 H), 1.34- 1.23 (m, 9 H), 1.21-1.01 (m, 3 H) ppm. |
| 124 | 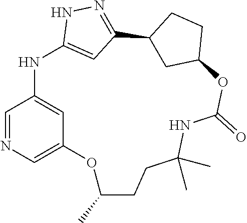 [SMILES: C[C@H]1CCC(C)(C)NC(=O)O[C@@H]2CC[C@@H](C2)c2cc([nH]n2)Nc2cncc(c2)O1] | LCMS: 400.2 [M + H]⁺; ¹H NMR (400 MHz, DMSO-d₆) δ 12.01-11.95 (m, 1 H), 8.55-8.41 (m, 1 H), 7.77 (s, 1 H), 7.52 (s, 1 H), 6.98-6.69 (m, 1 H), 6.67-6.62 (m, 1 H), 5.97-5.95 (m, 1 H), 4.93-4.987 (m, 1 H), 4.45- 4.43 (m, 1 H), 3.20 (s, 1 H), 2.00- 1.84 (m, 2 H), 1.81-1.78 (m, 5 H), 1.46-1.44 (m, 2 H), 1.23-1.14 (m, 10 H) ppm. |
| 128 | 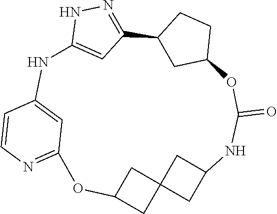 [SMILES: O=C1NC2CC3(C2)CC(C3)Oc2cc(ccn2)Nc2cc(n[nH]2)[C@H]2CC[C@H](C2)O1] | LCMS: 396.3 [M + H]⁺; ¹H NMR (400 MHz, DMSO-d₆) δ 12.32-12.29 (m, 1H), 8.89 (s, 1H), 7.78 (d, J = 4.4, 1H), 6.99 (d, J = 4.8, 1H), 6.46 (d, J = 3.2, 1H), 6.31 (d, J = 5.6, 1H), 6.02 (s, 1H), 4.99-4.97 (m, 1H), 4.75- 4.74 (m, 1H), 3.62-3.59 (m, 1H), 3.17- 3.09 (m, 1H), 2.56-2.51 (m, 1H), 2.49-2.47 (m, 1H), 2.17-1.97 (m, 9H), 1.92-1.73 (m, 3H) ppm. |
| 136 | 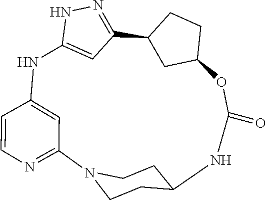 [SMILES: O=C1NC2CCN(CC2)c2cc(ccn2)Nc2cc(n[nH]2)[C@H]2CC[C@H](C2)O1] | LCMS: 369.4 [M + H]⁺; ¹H NMR (400 MHz, DMSO-d6) δ 8.57 (s, 1H), 8.16 (s, 1H), 7.72 (d, J = 4.8 Hz, 1H), 7.01- 6.99 (m, 1H), 6.82 (d, J = 2.8 Hz, 1H), 6.29-6.27 (m, 1H), 5.67 (d, J = 8.8 Hz, 1H), 5.04-5.02 (m, 1H), 4.29- 4.25 (m, 1H), 3.95-3.85 (m, 1H), 3.56-3.53 (m, 1H), 3.15-3.12 (m, 2H), 2.92-2.85 (m, 2H), 2.05-2.00 (m, 1H), 1.80-1.63 (m, 6H), 1.27- 1.24 (m, 2H) ppm. |
| 137 | 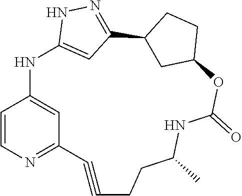 [SMILES: C[C@@H]1CCC#Cc2cc(ccn2)Nc2cc(n[nH]2)[C@H]2CC[C@H](C2)OC(=O)N1] | LCMS: 366.2 [M + H]⁺; ¹H NMR (400 MHz, DMSO-d6) δ 12.10 (s, 1 H), 9.07 (s, 1 H), 8.21 (s, 1 H), 8.03 (s, 1 H), 7.07 (s, 1 H), 6.71 (s, 2 H), 6.06- 6.03 (m, 1 H), 5.25-5.23 (m, 1H), 3.71 (m, 1 H), 3.37-3.35 (m, 2 H), 2.54-2.51 (m, 1 H), 2.34-2.33 (m, 1 H), 1.82-1.79 (m, 5 H), 1.51 (s, 1 H), 1.10-1.08 (d, 3 H) ppm. |
| 141 | 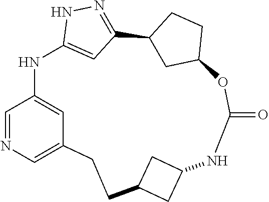 | LCMS: 368.2 [M + H]⁺; ¹H NMR (400 MHz, DMSO-d₆) δ 12.78-11.79 (m, 1H), 9.11-8.63 (m, 1H), 8.08 (d, J = 2.3 Hz, 1H), 7.90 (s, 1H), 7.62-7.42 (m, 1H), 7.37-6.98 (m, 1H), 6.06- 5.90 (m, 1H), 5.10-4.90 (m, 1H), 4.16-3.94 (m, 1H), 4.25-3.84 (m, 1H), 3.74-3.59 (m, 1H), 3.20-3.12 (m, 2H), 2.23-1.94 (m, 4H), 1.92- 1.81 (m, 4H), 1.80-1.59 (m, 5H) ppm. |
| 142 | 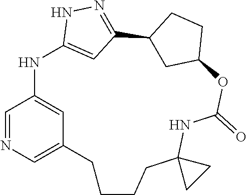 [SMILES: O=C1NC2(CCCCc3cncc(c3)Nc3cc(n[nH]3)[C@H]3CC[C@H](C3)O1)CC2] | LCMS: 382.3 [M + H]⁺; ¹H NMR (400 MHz, DMSO-d₆) δ 8.40-8.23 (m, 1H), 8.01 (d, J = 2.4 Hz, 1H), 7.74 (s, 1H), 7.40 (s, 1H), 7.20 (s, 1H), 5.85 (s, 1H), 5.05-4.87 (m, 1H), 3.22- 3.10 (m, 1H), 2.65-2.58 (m, 1H), 2.47-2.36 (m, 2H), 2.10-1.93 (m, 2H), 1.77-1.69 (m, 4H), 1.63-1.40 (m, 2H), 1.33-1.18 (m, 1H), 0.97- 0.82 (m, 1H), 0.58-0.37 (m, 4H) ppm. |
| 143 | 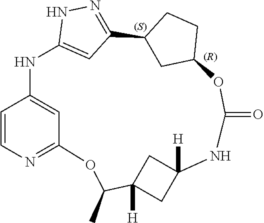 | LCMS: 384.2 [M + H]⁺; ¹H NMR (400 MHz, DMSO-d₆) δ 9.09-8.74 (m, 1 H), 7.83-7.66 (m, 1 H), 6.88-6.71 (m, 1 H), 6.57-6.38 (m, 2 H), 6.13- 5.97 (m, 1 H), 5.02 (br s, 1 H), 4.81 (br d, J = 5.88 Hz, 1 H), 3.86 (br d, J = 5.13 Hz, 1 H), 3.26 (br s, 1 H), 2.45-2.39 (m, 1 H), 2.28-2.00 (m, 5 H), 1.88-1.72 (m, 4 H), 1.67-1.58 (m, 1 H), 1.15 (br d, J = 5.88 Hz, 3 H) ppm. |
| 144 | 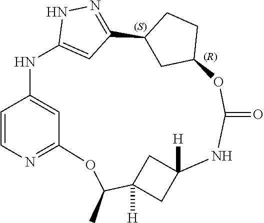 | LCMS: 384.2 [M + H]⁺; ¹H NMR (400 MHz, DMSO-d₆) δ 12.40-11.17 (m, 1 H), 8.97-8.84 (m, 1 H), 7.74 (d, J = 5.63 Hz, 1 H), 7.06 (d, J = 9.26 Hz, 1 H), 6.47-6.38 (m, 2 H), 6.02 (s, 1 H), 5.31-5.22 (m, 1 H), 5.22-5.14 (m, 1 H), 4.35-4.22 (m, 1 H), 3.24- 3.20 (m, 1 H), 2.45-2.35 (m, 2 H), 2.15-2.05 (m, 2 H), 2.00-1.84 (m, 3 H), 1.78-1.62 (m, 4 H), 1.17 (d, J = 6.38 Hz, 3 H) ppm. |
| 145 | 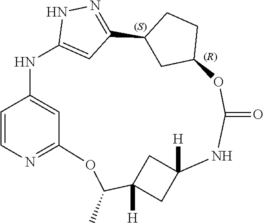 | LCMS: 384.2 [M + H]⁺; ¹H NMR (400 MHz, DMSO-d₆) δ 8.83 (br s, 1 H), 7.72 (d, J = 5.75 Hz, 1 H), 6.50 (d, J = 6.50 Hz, 1 H), 6.45-6.37 (m, 2 H), 6.19 (s, 1 H), 5.13-5.04 (m, 1 H), 5.02-4.94 (m, 1 H), 3.92-3.77 (m, 1 H), 2.31-2.17 (m, 3 H), 2.13-2.00 (m, 3 H), 1.92-1.72 (m, 5 H), 1.62- 1.50 (m, 1 H), 1.11 (d, J = 6.25 Hz, 3 H) ppm. |
| 147 | 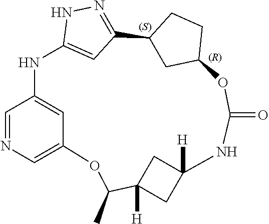 | LCMS: 384.3 [M + H]⁺; ¹H NMR (400 MHz, DMSO-d₆) δ 8.73-8.54 (m, 1 H), 7.84 (d, J = 1.50 Hz, 1 H), 7.65 (d, J = 2.00 Hz, 1 H), 7.36-7.05 (m, 2 H), 5.97 (br s, 1 H), 5.00 (br s, 1 H), 4.34 (br s, 1 H), 3.86-3.71 (m, 1 H), 3.24- 3.18 (m, 1 H), 2.46-2.05 (m, 6 H), 1.86-1.60 (m, 5 H), 1.17 (d, J = 6.00 Hz, 3 H) ppm. |
| 148 | 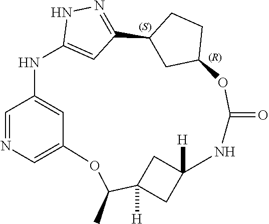 | LCMS: 384.3 [M + H]⁺; ¹H NMR (400 MHz, DMSO-d₆) δ 8.80-8.55 (m, 1 H), 7.87 (d, J = 2.13 Hz, 1 H), 7.60 (d, J = 2.25 Hz, 1 H), 7.22-7.01 (m, 2 H), 5.96 (s, 1 H), 5.01 (br d, J = 3.25 Hz, 1 H), 4.39 (br t, J = 6.32 Hz, 1 H), 4.33- 4.20 (m, 1 H), 3.24-3.15 (m, 1 H), 2.40 (ddd, J = 15.04, 10.91, 6.94 Hz, 1 H), 2.30-2.05 (m, 3 H), 2.01-1.68 (m, 7 H), 1.15 (d, J = 6.00 Hz, 3 H) ppm. |
| 149 | 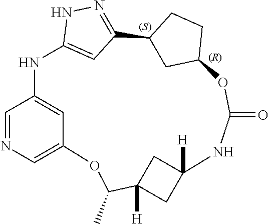 | LCMS: 384.2 [M + H]⁺; ¹H NMR (400 MHz, DMSO-d₆) δ 8.71-8.53 (m, 1 H), 7.93-7.80 (m, 1 H), 7.58 (d, J = 1.63 Hz, 1 H), 7.29-7.00 (m, 1 H), 6.06-5.88 (m, 1 H), 4.98 (br s, 1 H), 4.50 (br d, J = 3.88 Hz, 1 H), 3.90- 3.75 (m, 1 H), 3.24-3.19 (m, 1 H), 2.32-1.97 (m, 4 H), 1.95-1.53 (m, 8 H), 1.08 (br d, J = 5.38 Hz, 3 H) ppm. |
| 150 | 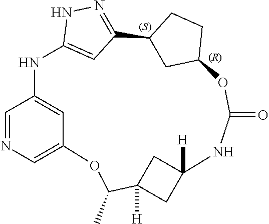 | LCMS: 384.3 [M + H]⁺; ¹H NMR (400 MHz, DMSO-d₆) δ 12.55-10.69 (m, 1 H), 8.82-8.52 (m, 1 H), 7.84 (d, J = 1.25 Hz, 1 H), 7.54 (d, J = 1.50 Hz, 1 H), 7.03-7.25 (m, 2 H), 6.01 (s, 1 H), 5.15 (br s, 1 H), 4.46 (br d, J = 6.63 Hz, 1 H), 4.33-4.18 (m, 1 H), 3.25-3.16 (m, 1 H), 2.41 (ddd, J = 15.01, 11.38, 6.13 Hz, 1 H), 2.24- 2.02 (m, 4 H), 1.98-1.74 (m, 4 H), 1.73-1.60 (m, 2 H), 1.14 (br d, J = 5.75 Hz, 3 H) ppm. |
| 151 | 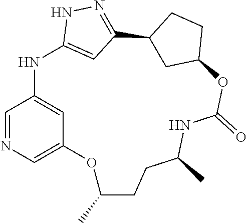 [SMILES: C[C@H]1CC[C@H](C)Oc2cncc(c2)Nc2cc(n[nH]2)[C@H]2CC[C@H](C2)OC(=O)N1] | LCMS: 386.1 [M + H]⁺; ¹H NMR (400 MHz, DMSO-d₆) δ 12.42-11.60 (m, 1H), 8.65-8.45 (m, 1H), 8.32 (s, 1H), 7.91-7.73 (m, 1H), 7.69-7.46 (m, 1H), 7.04-6.60 (m, 2H), 5.99- 5.73 (m, 1H), 5.10-4.88 (m, 1H), 4.49-4.02 (m, 1H), 3.59-3.43 (m, 1H), 3.24-3.15 (m, 1H), 2.47-2.31 (m, 1H), 2.14-1.98 (m, 1H), 1.97- 1.60 (m, 5H), 1.54-1.17 (m, 6H), 1.07-0.89 (m, 3H) ppm. |
| 152 | 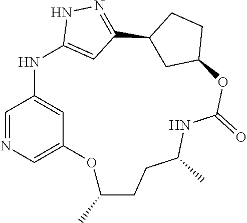 [SMILES: C[C@@H]1CC[C@H](C)Oc2cncc(c2)Nc2cc(n[nH]2)[C@H]2CC[C@H](C2)OC(=O)N1] | LCMS: 386.2 [M + H]⁺; ¹H NMR (400 MHz, DMSO-d₆) δ 12.13 (br d, J = 18.5 Hz, 1H), 8.53 (s, 1H), 8.35- 8.32 (m, 1H), 7.83-7.72 (m, 1H), 7.57-7.48 (m, 1H), 7.08-6.60 (m, 2H), 5.92 (s, 1H), 5.03-4.82 (m, 1H), 4.66-4.22 (m, 1H), 3.70-3.44 (m, 1H), 2.39-2.09 (m, 2H), 1.91- 1.68 (m, 5H), 1.60-1.43 (m, 3H), 1.25-1.16 (m, 1H), 1.16-0.93 (m, 6H) ppm. |
| 153 | 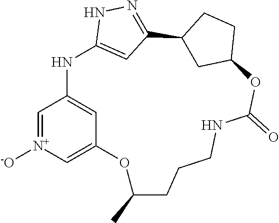 | LCMS: 388.3 [M + H]⁺; ¹H NMR (400 MHz, DMSO-d₆) δ 12.19 (br, 1H), 8.73-8.71 (m, 1H), 7.50-7.49 (s, 1H), 7.27-7.25 (m, 1H), 6.65-6.62 (m, 1H), 6.93-6.91 (m, 1H), 5.22- 5.02 (s, 1H), 4.52-4.41 (m, 1H), 3.22- 3.15 (m, 1H), 2.71-2.69 (m, 2H), 2.08-2.02 (m, 1H), 1.85-1.81 (m, 5H), 1.52-1.49 (m, 1H), 1.42-1.32 (m, 2H), 1.24-1.11 (m, 2H) ppm. |
| 154 | 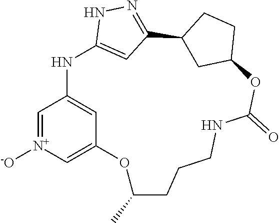 | LCMS: 388.3 [M + H]⁺; ¹H NMR (400 MHz, DMSO-d₆) δ 12.34-11.91 (m, 1H), 8.79 (d, J = 15.6 Hz, 1H), 7.51 (d, J = 7.2 Hz, 1H), 7.36-6.82 (m, 2H), 6.63 (s, 1H), 5.94 (d, J = 9.2 Hz, 1H), 4.93 (d, J = 5.6 Hz, 1H), 4.44 (d, J = 5.6 Hz, 1H), 4.40 (d, J = 2.8 Hz, 2H), 3.26-3.19 (m, 2H), 3.05 (s, 1H), 2.34-2.25 (m, 1H), 2.05-1.87 (m, 2H), 1.80-1.68 (m, 4H), 1.27 (d, J = 2.8 Hz, 3H), 1.19 (s, 1H) ppm. |
| 155 | 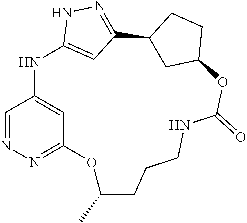 [SMILES: C[C@H]1CCCNC(=O)O[C@@H]2CC[C@@H](C2)c2cc([nH]n2)Nc2cnnc(c2)O1] | LCMS: 373.2 [M + H]⁺; ¹H NMR (400 MHz, DMSO-d₆) δ 12.26-12.21 (m, 1 H), 9.43-9.32 (m, 1 H), 8.50-8.26 (m, 1 H), 7.01-6.94 (m, 1 H), 6.76- 6.44 (m, 1 H), 6.35-6.34 (m, 1 H), 6.03 (s, 1 H), 5.03-4.95 (m, 2 H), 3.47-3.27 (m, 2 H), 3.26-3.24 (m, 1 H), 3.22-3.19 (m, 1 H), 2.51-2.49 (m, 1 H), 1.86 (s, 1 H), 1.78-1.77 (m, 5 H), 1.52-1.50 (m, 2 H), 1.33- 1.31 (m, 3 H) ppm. |
| 156 | 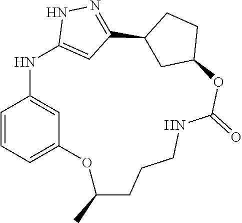 [SMILES: C[C@@H]1CCCNC(=O)O[C@@H]2CC[C@@H](C2)c2cc([nH]n2)Nc2cccc(c2)O1] | LCMS: 371.2 [M + H]⁺; ¹H NMR (400 MHz, DMSO-d₆) δ 8.24-8.13 (m, 1H), 8.07 (d, J = 9.2 Hz, 1H), 6.98 (t, J = 7.6 Hz, 1H), 6.81 (d, J = 8.4 Hz, 1H), 6.64 (s, 1H), 6.47-6.35 (m, 1H), 6.17 (dd, J = 1.6, 8.0 Hz, 1H), 5.88 (s, 1H), 5.21-4.96 (m, 1H), 4.43-4.23 (m, 1H), 3.18-3.13 (m, 2H), 2.74 (dd, J = 3.2, 6.0 Hz, 1H), 2.12-1.94 (m, 2H), 1.86-1.68 (m, 6H), 1.50-1.40 (m, 2H), 1.24-1.16 (m, 3H) ppm. |
| 157 | 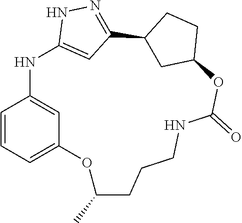 [SMILES: C[C@H]1CCCNC(=O)O[C@@H]2CC[C@@H](C2)c2cc([nH]n2)Nc2cccc(c2)O1] | LCMS: 371.2 [M + H]⁺; ¹H NMR (400 MHz, DMSO-d₆) δ 12.02-11.68 (m, 1H), 8.33-8.06 (m, 1H), 7.06-6.95 (m, 1H), 6.86-6.76 (m, 1H), 6.63 (s, 1H), 6.48-6.36 (m, 1H), 6.15 (dd, J = 1.6, 3.6 Hz, 1H), 5.93-5.82 (m, 1H), 5.08-4.82 (m, 1H), 4.43-4.17 (m, 1H), 3.21-3.12 (m, 2H), 2.91- 2.80 (m, 1H), 2.07-1.90 (m, 2H), 1.83-1.67 (m, 4H), 1.61-1.28 (m, 4H), 1.23 (d, J = 4.8 Hz, 3H) ppm. |
| 171 | 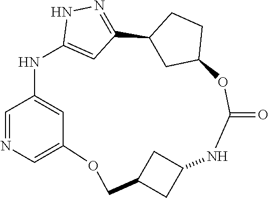 | LCMS: 370.4 [M + H]⁺; ¹H NMR (400 MHz, DMSO-d₆) δ 8.64 (S, 1H), 7.83 (S, 1H), 7.57 (d, J = 6.0 Hz, 1H), 7.18 (s, 1H), 7.15 (m, 1H), 5.99 (s, 1H), 5.09 (s, 1H), 4.26-4.22 (m, 2H), 4.15- 4.11 (m, 1H), 3.17 (s, 1H), 2.41- 2.38 (m, 2H), 2.03-2.00 (m, 5H), 1.83-1.81 (m, 2H), 1.73-1.70 (m, 2H), 1.02 (d, J = 4.8 Hz, 1H) ppm. |
| 172 | 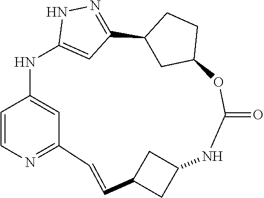 | LCMS: 366.2 [M + H]⁺; ¹H NMR (400 MHz, DMSO-d₆) δ 9.24-8.70 (m, 1H), 8.06 (s, 1H), 7.37 (br d, J = 1.0 Hz, 1H), 7.24-7.17 (m, 1H), 6.73 (dd, J = 10.2, 16.0 Hz, 1H), 6.62 (dd, J = 1.8, 5.4 Hz, 1H), 6.38-6.29 (m, 1H), 6.26 (s, 1H), 5.11-5.02 (m, 1H), 4.70-4.50 (m, 1H), 3.19-3.14 (m, 1H), 2.77-2.70 (m, 1H), 2.61- 2.54 (m, 1H), 2.22-2.10 (m, 2H), 2.10-1.87 (m, 4H), 1.86-1.74 (m, 3H), 1.71 (br s, 1H) ppm. |
| 175 | 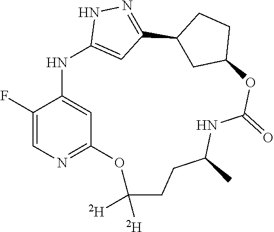 [SMILES: [2H]C1([2H])CC[C@H](C)NC(=O)O[C@@H]2CC[C@@H](C2)c2cc([nH]n2)Nc2cc(ncc2F)O1] | LCMS: 392.2 [M + H]⁺; ¹H NMR (400 MHz, DMSO-d₆) δ 12.43-12.23 (m, 1 H), 8.88-8.70 (m, 1 H), 7.85 (d, J = 3.18 Hz, 1 H), 6.90 (br d, J = 8.68 Hz, 1 H), 6.38 (d, J = 5.99 Hz, 1 H), 6.05 (s, 1 H), 5.02 (br s, 1 H), 3.70- 3.59 (m, 1 H), 3.24-3.18 (m, 1 H), 2.45-2.33 (m, 1 H), 2.16-2.01 (m, 1 H), 1.88-1.65 (m, 6 H), 1.56-1.37 (m, 2 H), 1.02 (br d, J = 6.60 Hz, 3 H) ppm. |
| 176 | 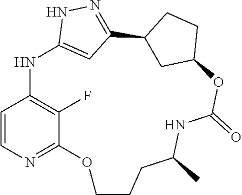 [SMILES: C[C@H]1CCCOc2nccc(c2F)Nc2cc(n[nH]2)[C@H]2CC[C@H](C2)OC(=O)N1] | LCMS: 390.2 [M + H]⁺; ¹H NMR (400 MHz, DMSO-d₆) δ 12.04 (s, 1H), 11.36-10.96 (m, 1H), 8.55 (d, J = 2.0 Hz, 1H), 7.02 (d, J = 7.2 Hz, 1H), 6.91 (d, J = 4.4 Hz, 1H), 5.78 (s, 1H), 5.08-4.96 (m, 1H), 3.85-3.69 (m, 1H), 3.28-3.20 (m, 2H), 3.11-3.03 (m, 1H), 2.06-1.98 (m, 1H), 1.96- 1.86 (m, 2H), 1.85-1.58 (m, 6H), 1.52-1.43 (m, 1H), 1.07 (d, J = 12.0 Hz, 3H) ppm. |
| 177 | 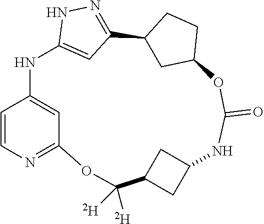 | LCMS: 372.2 [M + H]⁺; ¹H NMR (400 MHz, DMSO-d₆) δ 9.01 (1 H, s), 7.77- 7.76 (1 H, m), 7.06-7.03 (1 H, m), 6.51-6.46 (2 H, m), 6.07 (1 H, s), 5.14-5.12 (1 H, m), 4.32-4.25 (1 H, m), 3.32-3.22 (1 H, m), 2.50-2.39 (1 H, m), 2.12-2.09 (1 H, m), 2.08- 2.07 (3 H, m), 1.92-1.91 (3 H, m), 1.75-1.73 (3 H, m) ppm. |
| 179 | 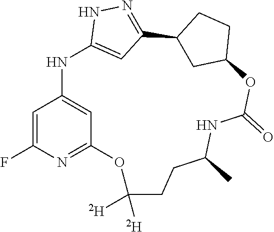 [SMILES: [2H]C1([2H])CC[C@H](C)NC(=O)O[C@@H]2CC[C@@H](C2)c2cc([nH]n2)Nc2cc(F)nc(c2)O1] | LCMS: 392.3 [M + H]⁺; ¹H NMR (400 MHz, DMSO-d₆) δ 12.18 (s, 1H), 9.23 (s, 1H), 7.06-6.87 (m, 1H), 6.20 (s, 1H), 6.01 (s, 2H), 5.02 (s, 1H), 3.75- 3.57 (m, 1H), 3.23-3.11 (m, 1H), 2.42-2.31 (m, 1H), 2.13-1.99 (m, 1H), 1.85-1.66 (m, 6H), 1.58-1.42 (m, 2H), 1.03 (d, J = 6.6 Hz, 3H) ppm. |
| 180 | 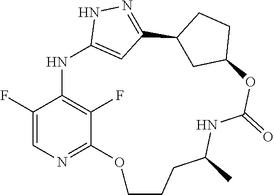 [SMILES: C[C@H]1CCCOc2ncc(F)c(c2F)Nc2cc(n[nH]2)[C@H]2CC[C@H](C2)OC(=O)N1] | LCMS: 408.2 [M + H]⁺; ¹H NMR (400 MHz, DMSO-d₆) δ 12.04-11.82 (m, 1H), 8.25 (s, 1H), 7.34 (d, J = 5.6 Hz, 1H), 5.70 (s, 1H), 5.02 (s, 1H), 3.79- 3.77 (m, 1H), 3.28-3.22 (m, 3H), 3.12-3.04 (m, 1H), 2.45-2.38 (m, 1H), 2.06-2.01 (m, 1H), 1.93-1.86 (m, 2H), 1.85-1.69 (m, 4H), 1.68- 1.63 (m, 1H), 1.53-1.49 (m, 1H), 1.10-1.04 (m, 3H) ppm. |
| 190 | 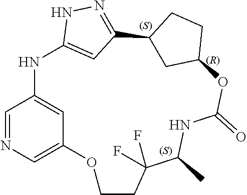 | LCMS: 408.3 [M + H]⁺; ¹H NMR (400 MHz, DMSO-d₆) δ 12.09-11.89 (m, 1H), 8.63-8.49 (m, 1H), 7.83 (br d, J = 1.3 Hz, 1H), 7.61-7.48 (m, 2H), 7.02-6.94 (m, 1H), 5.99-5.86 (m, 1H), 5.13-5.04 (m, 1H), 4.41-4.29 (m, 1H), 4.21-4.09 (m, 1H), 4.05- 3.89 (m, 1H), 3.23-3.15 (m, 1H), 2.42 (dd, J = 5.1, 14.8 Hz, 2H), 2.12- 1.94 (m, 2H), 1.87-1.71 (m, 4H), 1.14 (br d, J = 6.9 Hz, 3H) ppm. |
| 218 | 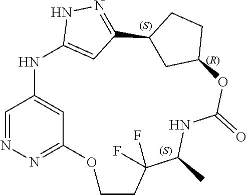 | LCMS: 409.3 [M + H]⁺; ¹H NMR (400 MHz, DMSO-d₆) δ 12.44-12.17 (m, 1H), 9.62-9.31 (m, 1H), 8.50 (s, 1H), 7.45 (br d, J = 8.4 Hz, 1H), 6.50 (s, 1H), 6.22-5.97 (m, 1H), 5.25- 5.00 (m, 1H), 4.52-4.25 (m, 2H), 4.13-3.90 (m, 1H), 3.30-3.20 (m, 1H), 2.49-2.16 (m, 3H), 2.14-2.02 (m, 1H), 1.88-1.70 (m, 4H), 1.14 (br d, J = 6.6 Hz, 3H) ppm. |
| 220 | 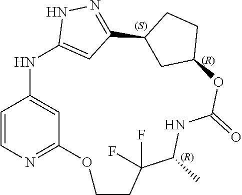 | LCMS: 408.2 [M + H]⁺; ¹H NMR (400 MHz, DMSO-d₆) δ 12.53-12.08 (m, 1H), 8.95 (s, 1H), 8.13 (s, 1H), 7.76 (d, J = 5.7 Hz, 1H), 7.33-6.86 (m, 1H), 6.52-6.28 (m, 2H), 6.14 (s, 1H), 5.27-4.83 (m, 1H), 4.36-4.13 (m, 2H), 4.09-3.88 (m, 1H), 3.30- 3.03 (m, 2H), 2.43-2.06 (m, 3H), 1.98-1.70 (m, 4H), 1.11 (d, J = 7.0 Hz, 3H) ppm. |
| 221 | 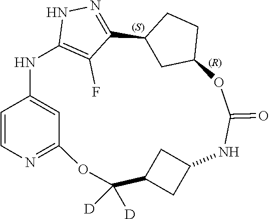 | LCMS: 390.3 [M + H]⁺; ¹H NMR (400 MHz, DMSO-d₆) δ 12.55-12.13 (m, 1 H), 8.63 (s, 1 H), 8.15 (s, 1 H), 7.75 (d, J = 5.7 Hz, 1 H), 6.94 (d, J = 9.1 Hz, 1 H), 6.44 (dd, J = 5.8, 1.9 Hz, 1 H), 5.80 (br d, J = 1.2 Hz, 1 H), 5.03- 5.16 (m, 1 H), 4.16 (dq, J = 16.9, 8.3 Hz, 1 H), 2.34 (ddd, J = 15.2, 10.0, 7.5 Hz, 1 H), 2.24 (br d, J = 3.7 Hz, 1 H), 2.01-2.15 (m, 5 H), 1.83-2.01 (m, 4 H), 1.66-1.81 (m, 1 H) ppm. |
| 222 | 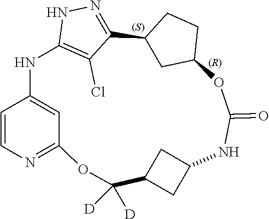 | LCMS: 406.2 [M + H]⁺; ¹H NMR (400 MHz, DMSO-d₆) δ 12.88-12.66 (m, 1 H), 8.53 (br s, 1 H), 8.15 (s, 1 H), 7.81-7.71 (m, 1 H), 6.94 (br d, J = 8.6 Hz, 1 H), 6.46 (br d, J = 4.6 Hz, 1 H), 5.69-5.49 (m, 1 H), 5.07 (br s, 1 H), 4.02 (br d, J = 5.8 Hz, 1 H), 3.16 (br s, 1 H), 2.37-2.19 (m, 4 H), 2.05- 1.89 (m, 6 H), 1.75 (br d, J = 3.2 Hz, 1 H) ppm. |
| 228 | 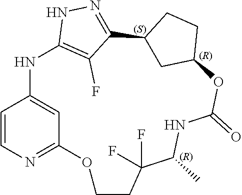 | LCMS: 426.2 [M + H]⁺; ¹H NMR (400 MHz, DMSO-d₆) δ 8.45 (s, 1H), 8.15 (s, 1H), 7.79 (d, J = 5.6 Hz, 1H), 6.59- 6.38 (m, 2H), 5.84-5.67 (m, 1H), 5.13 (d, J = 2.4 Hz, 1H), 4.32-4.11 (m, 2H), 4.06-3.86 (m, 1H), 3.32 (s, 1H), 2.46-2.36 (m, 1H), 2.35-2.21 (m, 3H), 2.13 (d, J = 14.4 Hz, 2H), 1.99-1.86 (m, 1H), 1.85-1.73 (m, 1H), 1.07 (d, J = 6.8 Hz, 3H) ppm. |
| 233 | 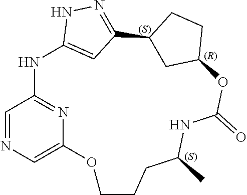 | LCMS: 373.2 [M + H]⁺; ¹H NMR (400 MHz, DMSO-d₆) δ 12.40-11.62 (m, 1H), 9.94-9.61 (m, 1H), 7.76-7.70 (m, 1H), 7.43 (s, 1H), 7.08-6.67 (m, 1H), 6.70 (br d, J = 7.2 Hz, 1H), 6.60- 6.48 (m, 1H), 5.02 (br s, 1H), 4.71- 4.44 (m, 1H), 4.05 (br dd, J = 7.1, 9.3 Hz, 1H), 3.80-3.64 (m, 1H), 3.55- 3.43 (m, 1H), 3.18 (br d, J = 6.6 Hz, 1H), 2.50-2.32 (m, 1H), 2.07-1.96 (m, 1H), 1.84-1.74 (m, 3H), 1.72- 1.59 (m, 2H), 1.58-1.46 (m, 1H), 1.57-1.21 (m, 1H), 1.11-0.94 (m, 3H) ppm. |
| 234 | 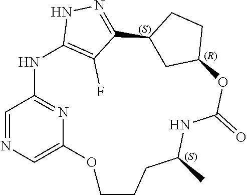 | LCMS: 391.3 [M + H]⁺; ¹H NMR (400 MHz, DMSO-d₆) δ 12.21-12.00 (m, 1H), 9.32-9.12 (m, 1H), 8.54-8.42 (m, 1H), 7.66 (br s, 1H), 7.62-7.62 (m, 1H), 7.45 (s, 1H), 6.83-6.53 (m, 1H), 5.11-4.91 (m, 1H), 4.72-4.27 (m, 1H), 3.95-3.75 (m , 1H), 3.75- 3.59 (m, 1H), 3.22-3.12 (m, 1H), 2.47-2.28 (m, 2H), 2.26-1.94 (m, 3H), 1.92-1.83 (m, 2H), 1.81-1.75 (m, 1H), 1.75 (br s, 1H), 1.61-1.37 (m, 3H), 1.12-0.89 (m, 4H) ppm. |
| 242 | 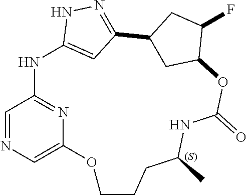 | LCMS: 391.2 [M + H]⁺; ¹H NMR (400 MHz, DMSO-d₆) δ 11.88-11.65 (m, 1H), 9.49-9.32 (m, 1H), 7.75 (s, 1H), 7.46 (br d, J = 2.3 Hz, 1H), 7.00- 6.46 (m, 2H), 5.18-4.91 (m, 2H), 4.62-3.99 (m, 2H), 3.80-3.55 (m, 1H), 3.27 (s, 1H), 2.41-2.33 (m, 1H), 2.14-2.00 (m, 1H), 1.90-1.75 (m, 3H), 1.69-1.51 (m, 2H), 1.35- 1.23 (m, 1H), 1.07 (d, J = 6.8 Hz, 3H) ppm. |
| 243 | 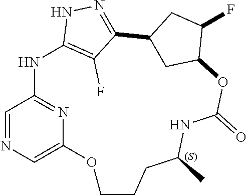 | LCMS: 409.2 [M + H]⁺; ¹H NMR (400 MHz, DMSO-d₆) δ 12.01-11.77 (m, 1H), 9.01-8.79 (m, 1H), 7.69 (s, 1H), 7.51-7.32 (m, 1H), 6.66-6.26 (m, 1H), 5.30-4.85 (m, 2H), 4.32- 3.89 (m, 1H), 3.71 (s, 1H), 3.34- 3.15 (m, 1H), 2.44-2.39 (m, 2H), 1.77-1.62 (m, 2H), 1.54-1.40 (m, 2H), 1.36-1.23 (m, 3H), 1.01 (br d, J = 5.9 Hz, 3H) ppm. |
| 244 | 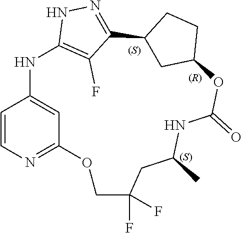 | LCMS: 426.3 [M + H]⁺; ¹H NMR (400 MHz, DMSO-d₆) δ 12.42-12.21 (m, 1H), 8.86 (s, 1H), 8.23 (s, 1H), 7.81 (d, J = 5.8 Hz, 1H), 7.02 (d, J = 8.4 Hz, 1H), 6.58-6.39 (m, 1H), 5.74 (s, 1H), 4.97 (d, J = 2.8 Hz, 1H), 4.40- 4.24 (m, 1H), 4.17-4.05 (m, 1H), 3.81-3.75 (m, 1H), 2.40-2.31 (m, 1H), 2.26-2.11 (m, 2H), 2.09-1.92 (m, 3H), 1.89-1.80 (m, 2H), 1.10 (br d, J = 6.8 Hz, 3H) ppm. |

### CDK Protein Kinase Inhibition by ADP-Glo Assay

This ADP-Glo assay measures ADP formed from a kinase reaction and indirectly quantifies the phosphorylation of peptide substrates by the CDK/cyclin protein complexes. The typical assays were carried out in Nanosyn by combining kinase/cyclin complexes, substrates, compounds, and cofactors (ATP and Mg2+) in a well of a 384-well microtiter plate (Corning 3824) and incubating at 22° C. (Table 1). At the end of the incubation, the reaction is quenched, and conversion is detected by luminescence using the Promega ADP-Glo™ Kinase Assay kit (catalog #V9102). Briefly, 5 μL of 1×enzyme and substrate buffer (or control), 50 nL of 100× compound, and 0.5 uL of 10 mM ATP were sequentially added to a well of a 384-well plate. The assay plate was incubated at 22° C. according to assay conditions shown in Table 5.

Final assay buffer mixture contains:
100 mM HEPES, pH 7.5
0.1% BSA
0.01% Triton X-100
1 mM DTT
5 mM MgCl2
10 μM Sodium Orthovanadate
100 μM Beta-Glycerophosphate
1000 μM Ultra Pure ATP (provided in the Promega ADP-Glo™ Kinase Assay kit)
1% DMSO (from compound)

Upon completion, 5 μL of the ADP Glo reagent was added to each well and the plate was further incubated at room temperature for 45 minutes. 10 uL of the Kinase Detection Reagent was subsequently added to each well. After 10 minutes of incubation, the plate was read and analyzed on the BioTek Synergy microplate reader using BioTek Gen5 software.

TABLE 5
CDK Assay Conditions

| | | | Kinase | Substrate | |
|---|---|---|---|---|---|
| | Vendor | | Conc | Conc | Incubation |
| Kinase | Catalog | Substrate | (nM) | (nM) | Time (hr) |
| CDK1- | Millipore 14- | FAM-PKTPKKAKKL-OH | 0.6 | 2.5 | 4 |
| CYCLIN | 450 | | | | |
| B | | | | | |
| CDK2- | Millipore 14- | FAM-PKTPKKAKKL-OH | 8 | 10 | 4 |
| CYCLIN | 475 | | | | |
| E1 | | | | | |
| CDK4- | ThermoFisher | Ac-RRFRPASPLRGPPK-NH2 | 5 | 5 | 17 |
| CYCLIN | PV4400 | | | | |
| D1 | | | | | |
| CDK6- | Carna 04-107 | Ac-RRFRPASPLRGPPK-NH2 | 8 | 5 | 17 |
| CYCLIN | | | | | |
| D3 | | | | | |
| CDK9- | Carna 04-110 | FAM- | 6 | 5 | 17 |
| CYCLIN | | ACSYSPTSPSYSPTSPSYSPTSPSKK- | | | |
| T1 | | NH2 | | | |

### Phospho-Rb Expression Inhibition in OVCAR₃ Cells Quantified by in Cell Western

OVCAR₃ cells were obtained from American Type Culture Collection (ATCC, HTB-161). OVCAR₃ cells were plated in 96-well plates (VWR #10062-900, or Corning #3904) in 90 uL culture medium at a density of 20,000 cells/well in the RPM11640 growth medium containing 10% FBS and 1% Penicillin Streptomycin, and then incubated at 37° C. overnight. The following day, the test compound was administered to the cells by using 1000× compound stock solution prepared in DMSO at various concentrations. 1000× compound stock solution was first diluted in culturing medium to 10x, then 10 uL compound medium was added to each well in the cell plates. After administration of the compound, the cells were then incubated at 37° C. for 24 hours. Upon completion, the cells were washed with PBS briefly. 150 uL/well of 4% formaldehyde was added and the plates were incubated at room temperature for 20 mins. The cells were washed with PBS briefly, and permeabilized with 150 uL/well of ice cold 100% methanol for 10 mins. The cells were washed with PBS briefly and blocked with 100 uL/well LI-COR blocking buffer for 1 hr at room temperature with gentle shaking. The cells were incubated overnight at 4° C. with 50 uL primary antibody rabbit anti-Phospho-Rb (Ser807/811) (1:500, Cell Signaling, #8516) and anti-Rb (1:200, Santa Cruz, sc-73598) diluted in Intercept Blocking Buffer (LI-COR, #927-60001) containing 0.1% Tween 20. The next day, the cells were washed with 200 uL PBS containing 0.1% Tween 20, 5×5 mins at room temperature with gentle shaking, and incubated with 50 uL secondary antibody, IRDye® 800CW Goat anti-Rabbit IgG (1:1000, LI-COR, #926-32211) and IRDye® 680RD Goat anti-Mouse IgG (1:1000, LI-COR, #926-68070), in LI-COR blocking buffer with 0.2% Tween 20 for 1 hr at room temperature with gentle shaking. The cells were washed with 200 uL PBS containing 0.1% Tween 20, 5×5 mins at room temperature with gentle shaking. The cells were washed with PBS for 5 mins. 100 uL fresh PBS was added to each well and the plates were imaged on a LI-COR Odyssey CLX plate reader.

### Cell Growth Inhibition of CCNE1-Amplified OVCAR₃ Cells

OVCAR₃ cells were seeded in 96-well plates at 2000 cells/well in 90 μl of RPM11640 growth medium containing 10% FBS and 1% Penicillin Streptomycin, and then incubated at 37° C. overnight. The following day, the test compound was administered to the cells by using 1000× compound stock solution prepared in DMSO at various concentrations. 1000× compound stock solution was first diluted in culturing medium to 10x, then 10 uL compound medium was added to each well in the cell plates. After administration of the compound, the cells were then incubated at 37° C. for 6 days. Upon completion, the plates were equilibrated at room temperature for approximately 10 minutes. 100 ul of CellTiter-Glo® Reagent (Promega) was added to each well. The plates were then incubated at room temperature for 10 minutes and luminescence was recorded by EnSpire plate reader (PerkinElmer).

### Cell Growth Inhibition of CCNE1-Normal SKOV3 Cells

SKOV3 cells were obtained from American Type Culture Collection (ATCC, HTB-77). SKOV3 cells were seeded in 96-well plates at 1000 cells/well in 90 μl of RPM11640 growth medium containing 10% FBS and 1% Penicillin Streptomycin, and then incubated at 37° C. overnight. The following day, the test compound was administered to the cells by using 1000× compound stock solution prepared in DMSO at various concentrations. 1000× compound stock solution was first diluted in culturing medium to 10x, then 10 uL compound medium was added to each well in the cell plates. After administration of the compound, the cells were then incubated at 37° C. for 6 days. Upon completion, the plates were equilibrated at room temperature for approximately 10 minutes. 100 ul of CellTiter-Glo® Reagent (Promega) was added to each well. The plates were then incubated at room temperature for 10 minutes and luminescence was recorded by EnSpire plate reader (PerkinElmer).

TABLE 6
Inhibition of cellular Rb phosphorylation in OVCAR3 cells
and inhibition of growth in OVCAR3 and SKOV3 cells by
Exemplary Compounds against MCF7 and 22RV1 Cells

| | | | SKOV3 Cell |
|---|---|---|---|
| | | OVCAR3 Cell | Growth IC50 (A, ≤2 |
| | OVCAR-3 pRb | Growth IC50 (A, ≤2 | nM; B, >2 nM |
| | IC50 (A, ≤2 nM; | nM; B, >2 nM | and ≤10 nM; |
| | B, >2 nM and ≤10 | and ≤10 nM; | C, >10 nM and ≤50 |
| | nM; C, >10 nM | C, >10 nM and ≤50 | nM, D, >50 nM |
| | and ≤50 nM, | nM, D, >50 nM | and ≤250 nM, |
| | D, >50 nM and ≤250 | and ≤250 nM, | E, >250 nM and ≤1250 |
| Compound No. | nM, E, >250 nM) | E, >250 nM) | nM, F, >1250 nM) |
| 1 | C | C | F |
| 2 | E | E | N/A |
| 3 | C | D | F |
| 4 | C | D | E |
| 8 | B | C | F |
| 9 | C | C | F |
| 10 | C | D | F |
| 11 | C | D | F |
| 12 | B | C | E |
| 13 | B | B | E |
| 16 | C | C | F |
| 17 | B | B | E |
| 18 | D | D | F |
| 19 | B | C | F |
| 20 | C | C | F |
| 21 | B | B | E |
| 22 | A | A | C |
| 23 | A | B | D |
| 24 | E | E | N/A |
| 25 | E | E | N/A |
| 26 | C | C | E |
| 33 | C | D | F |
| 64 | A | B | D |
| 71 | B | C | D |
| 72 | C | E | F |
| 96 | A | B | D |
| 98 | A | A | C |
| 100 | B | C | D |
| 102 | A | B | D |
| 104 | B | C | F |
| 105 | C | D | F |
| 106 | A | A | C |
| 107 | A | A | C |
| 108 | B | B | E |
| 109 | B | C | E |
| 110 | D | E | F |
| 112 | D | E | N/A |
| 113 | A | B | C |
| 114 | C | C | E |
| 115 | C | C | E |
| 116 | C | C | E |
| 117 | A | A | D |
| 118 | A | A | C |
| 119 | B | C | F |
| 120 | B | C | F |
| 121 | C | D | F |
| 122 | A | B | E |
| 123 | E | E | N/A |
| 124 | A | B | C |
| 125 | B | C | E |
| 126 | C | D | E |
| 127 | E | E | N/A |
| 128 | B | B | E |
| 136 | E | E | N/A |
| 137 | C | D | F |
| 138 | C | D | F |
| 139 | D | D | F |
| 140 | B | C | D |
| 141 | A | B | D |
| 142 | B | C | E |
| 143 | D | D | N/A |
| 144 | B | B | E |
| 145 | D | D | F |
| 146 | C | C | E |
| 147 | D | D | F |
| 148 | A | B | E |
| 149 | C | C | F |
| 150 | B | B | E |
| 151 | B | B | E |
| 152 | B | C | E |
| 153 | E | E | N/A |
| 154 | D | E | N/A |
| 155 | C | D | F |
| 156 | E | E | N/A |
| 157 | C | C | E |
| 158 | C | C | E |
| 159 | B | B | D |
| 160 | D | D | N/A |
| 161 | A | A | D |
| 162 | B | C | E |
| 163 | C | C | E |
| 164 | A | A | C |
| 165 | B | C | D |
| 166 | B | C | E |
| 167 | B | C | E |
| 168 | C | D | F |
| 169 | C | D | F |
| 170 | B | B | D |
| 171 | A | B | E |
| 172 | A | B | C |
| 173 | C | D | F |
| 174 | D | D | F |
| 175 | E | E | N/A |
| 176 | B | C | E |
| 177 | B | C | N/A |
| 178 | C | D | N/A |
| 179 | C | D | F |
| 180 | E | E | N/A |
| 181 | C | C | E |
| 182 | B | C | E |
| 183 | C | D | F |
| 184 | E | E | N/A |
| 188 | C | D | F |
| 190 | B | C | E |
| 218 | C | E | F |
| 219 | A | A | C |
| 220 | B | C | E |
| 221 | C | C | N/A |
| 222 | D | E | N/A |
| 223 | B | C | E |
| 224 | B | C | F |
| 225 | B | C | E |
| 226 | B | N/A | N/A |
| 227 | C | N/A | N/A |
| 228 | D | D | F |
| 229 | B | C | N/A |
| 230 | C | C | N/A |
| 231 | C | C | N/A |
| 232 | C | D | N/A |
| 233 | A | A | N/A |
| 234 | B | C | N/A |
| 235 | A | A | N/A |
| 236 | B | C | N/A |
| 237 | C | D | N/A |
| 238 | C | D | N/A |
| 239 | C | N/A | N/A |
| 240 | C | N/A | N/A |
| 241 | A | A | N/A |
| 242 | A | N/A | N/A |
| 243 | A | N/A | N/A |
| 244 | C | D | F |
| 256 | B | C | N/A |

TABLE 7
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 105 | D | A | D | D | E |
| 104 | B | A | C | B | D |
| 13 | B | A | C | C | C |
| 4 | D | A | C | C | D |
| 9 | D | A | E | E | E |
| 10 | D | A | D | D | E |
| 16 | C | A | E | E | E |
| 17 | A | A | D | C | C |
| Control | E | B | E | E | E |

TABLE 8
CDK2 Fold Sensitivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 105 | C | D | C | B |
| 104 | D | C | D | B |
| 13 | E | C | D | C |
| 4 | D | E | E | D |
| 9 | C | B | B | B |
| 10 | D | C | C | B |
| 16 | E | B | B | B |
| 17 | E | B | B | C |
| Control | C | B | B | A |

TABLE 9
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 21 | E | B | D | D | D |
| 109 | B | A | C | D | D |
| Control | E | B | E | E | E |

TABLE 10
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 21 | D | D | D | E |
| 109 | D | B | B | B |
| Control | C | B | B | A |

TABLE 11
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 12 | B | A | D | D | D |
| 117 | A | A | B | B | B |
| 19 | C | A | E | E | E |
| 108 | B | A | D | D | D |
| Control | E | B | E | E | E |

TABLE 12
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 12 | D | B | B | A |
| 117 | E | E | E | D |
| 19 | D | A | B | A |
| 108 | B | B | A | A |
| Control | C | B | B | A |

TABLE 13
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 119 | B | A | D | B | D |
| 120 | B | A | C | B | C |
| 121 | D | A | E | D | D |
| 122 | A | A | C | B | B |
| 96 | A | A | C | B | A |
| Control | E | B | E | E | E |

TABLE 14
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 119 | D | C | E | C |
| 120 | E | C | E | C |
| 121 | D | B | C | B |
| 122 | E | C | D | D |
| 96 | E | D | E | E |
| Control | C | B | B | A |

TABLE 15
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 106 | A | A | B | B | A |
| 71 | C | A | D | D | D |
| Control | E | B | E | E | E |

TABLE 16
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 106 | E | E | E | E |
| 71 | E | D | D | D |
| Control | C | B | B | A |

TABLE 17
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 128 | B | A | B | B | B |
| 116 | C | A | B | B | C |
| 100 | B | A | D | D | B |
| 102 | A | A | D | D | B |
| 107 | A | A | B | B | A |
| Control | E | B | E | E | E |

TABLE 18
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 128 | E | D | E | E |
| 116 | D | E | E | D |
| 100 | E | C | C | D |
| 102 | E | C | C | E |
| 107 | E | E | E | E |
| Control | C | B | B | A |

TABLE 19
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 126 | D | C | D | D | D |
| 137 | D | B | E | E | E |
| 140 | B | A | B | C | B |
| 114 | D | A | C | D | D |
| 115 | C | B | B | B | D |
| 125 | D | A | D | D | D |
| 64 | A | A | B | B | B |
| Control | E | C | E | E | E |

TABLE 20
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 126 | E | E | E | E |
| 137 | E | E | D | D |
| 140 | E | E | E | E |
| 114 | E | E | E | D |
| 115 | E | E | E | E |
| 125 | E | E | D | D |
| 64 | E | D | D | E |
| Control | E | E | D | B |

TABLE 21
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 151 | A | A | C | C | D |
| 152 | B | A | C | C | D |
| 161 | A | A | B | B | B |
| 144 | D | A | D | D | D |
| 146 | D | A | E | D | E |
| 148 | B | A | C | C | D |
| 150 | B | A | D | C | D |
| 157 | C | A | D | D | D |
| 162 | B | A | D | D | D |
| Control | E | C | E | E | E |

TABLE 22
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 151 | E | D | D | D |
| 152 | E | D | E | D |
| 161 | E | E | E | E |
| 144 | E | D | D | C |
| 146 | E | C | C | C |
| 148 | E | E | D | D |
| 150 | E | D | D | B |
| 157 | E | D | D | C |
| 162 | E | C | C | C |
| Control | E | E | E | B |

TABLE 23
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 149 | D | B | D | E | E |
| 158 | D | B | D | E | E |
| 159 | B | A | B | C | C |
| 163 | C | A | D | D | D |
| Control | E | D | E | E | E |

TABLE 24
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 149 | E | E | D | C |
| 158 | E | E | D | D |
| 159 | E | E | E | E |
| 163 | E | D | D | D |
| Control | E | E | D | C |

TABLE 25
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 155 | B | A | D | D | E |
| 166 | B | A | C | B | B |
| 172 | A | A | A | B | A |
| Control | E | B | E | E | E |

TABLE 26
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 155 | E | D | D | B |
| 166 | E | C | D | D |
| 172 | E | E | E | E |
| Control | D | C | D | B |

TABLE 27
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1 |
| number | IC50 (nM) | IC50 (nM) | IC50 (nM) | IC50 (nM) | IC50 (nM) |
| 173 | D | B | D | E | E |
| 177 | B | A | C | D | D |
| Control | E | C | E | E | E |
| Control | E | C | E | E | E |

TABLE 28
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 173 | E | D | D | D |
| 177 | E | D | D | D |
| Control | E | D | D | C |
| Control | E | D | C | B |

TABLE 29
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 181 | D | A | E | E | D |
| 9 | D | B | E | E | E |
| Control | E | C | E | E | E |

TABLE 30
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 181 | D | C | C | D |
| 9 | E | C | C | C |
| Control | D | D | C | B |

TABLE 31
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 166 | B | A | D | D | B |
| 164 | A | A | A | B | A |
| 165 | B | B | B | B | C |
| 170 | A | A | B | B | B |
| Control | E | C | E | E | E |

TABLE 32
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 166 | E | D | D | E |
| 164 | E | E | E | E |
| 165 | E | E | E | E |
| 170 | E | E | E | E |
| Control | E | D | C | B |

TABLE 33
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 183 | E | B | E | E | E |
| Control | E | C | E | E | E |

TABLE 34
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 183 | D | C | C | B |
| Control | E | E | C | B |

TABLE 35
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 108 | B | A | B | C | C |
| 177 | B | A | B | B | C |
| 221 | C | A | D | D | E |
| 223 | C | A | D | D | D |
| 224 | B | A | D | D | E |
| 225 | A | A | B | B | B |
| Control | D | B | E | E | E |

TABLE 36
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 108 | E | E | D | D |
| 177 | E | E | E | D |
| 221 | E | C | C | B |
| 223 | E | C | C | C |
| 224 | E | C | C | B |
| 225 | E | E | E | E |
| Control | D | B | B | B |

TABLE 37
Selectivity of Exemplary Compounds against Representative
Recombinant CDK Kinases. A, ≤10 nM; B, >10 nM and ≤50
nM; C, >50 nM and ≤100 nM, D, >100 nM and ≤500
nM, E, >500 nM. The control compound is Pfizer's PF07104091 (Tagtociclib).

| | CDK1- | CDK2- | CDK4- | CDK6- | CDK9- |
|---|---|---|---|---|---|
| Compound | CYCLINB, | CYCLINE, | CYCLIND1, | CYCLIND3, | CYCLINT1, |
| number | IC50 (nM) | IC50 (nM) | IC50 (nM) | IC50 (nM) | IC50 (nM) |
| 229 | B | A | B | B | D |
| 230 | B | A | B | B | B |
| 231 | C | A | D | D | E |
| 232 | D | B | C | D | D |
| 244 | D | A | E | E | E |

TABLE 38
CDK2 Fold selectivity of Exemplary Compounds against Representative
Recombinant CDK Kinases. A, ≥500; B, >100 and ≤500;
C, >50 and ≤100, D, >20 and ≤50, E, <20.
The control compound is Pfizer's PF07104091 (Tagtociclib).

| Compound Number | CDK1 | CDK4 | CDK6 | CDK9 |
|---|---|---|---|---|
| 229 | E | E | E | C |
| 230 | E | E | E | E |
| 231 | E | B | B | B |
| 232 | E | E | E | E |
| 244 | D | B | B | A |

The embodiments of the disclosure may further be described using the following clauses:

1. A compound of Formula (II), a tautomer, a stereoisomer or a mixture of stereoisomers, a pharmaceutically acceptable salt, a hydrate, or a deuterated derivative thereof:

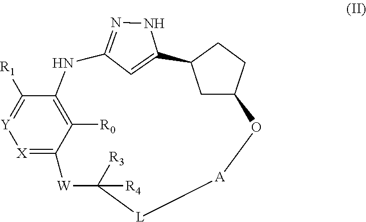

wherein:
A is a substituted or unsubstituted amide or a 5-12 membered aromatic ring, wherein the 5-12 membered aromatic ring is optionally substituted with 1, 2, 3, or 4 R_{A1};
   wherein R_{A1} is selected from hydrogen, —C(═O)—R_{A2}, C₁-C₅ alkyl, C₂-C₅ alkenyl, C₂-C₅ alkynyl, halo, C₁-C₅ haloalkyl, C₁-C₅ alkoxy, C₁-C₅ alkoxide, and —CN;
   wherein R_{A2} is selected from hydrogen, hydroxyl, C₁-C₅ alkyl, C₁-C₅ alkoxy, C₁-C₅ alkoxide, C₁-C₅ haloalkyl, C₂-C₅ alkenyl, and C₂-C₅ alkynyl;
R₀ is selected from hydrogen, halo, —CN, and C₁-C₅ alkyl;
R₁ is selected from hydrogen, halo, —CN, and C₁-C₅ alkyl;
R₃ and R₄ are independently absent or selected from hydrogen, C₁-C₅ alkyl, and halo, or R₃ and R₄, together with the carbon atom to which they are attached, form a carbonyl group, or a 3-6 membered cycloalkyl;
X and Y are independently N or CR₂;
   wherein R₂ is independently selected from hydrogen, haloalkyl, C₁-C₅ alkyl, C₁-C₅ alkoxy, and C₃-C₅ cycloalkyl;
W is absent or selected from alkylene, alkyne, —C(═O)—R₆—, —O—, —R₆—O—, —O—R₆—, —N(R₇)—, —NH—C(═O)—; —C(═O)— N(R₆)—, —S—, —S(═O)₂—, —P(R₇)₂—, 3-6 membered cycloalkyl, and 3-6 membered heterocycle, wherein the alkylene, 3-6 membered cycloalkyl, or 3-6 membered heterocycle are optionally substituted with 1, 2, 3, 4, or 5 R_{B};
   wherein each R₆ is independently selected from C₁-C₅ alkylene, 3-6 membered heterocycle, and 3-6 membered cycloalkyl;
   wherein each R₇ is independently selected from hydrogen, oxo, and C₁-C₅ alkyl;
   wherein each R_{B} is independently selected from hydrogen, oxo, halo, and C₁-C₅ alkyl;
and
L is absent or a linker of 1 to 10 carbon atoms in length, wherein one or more carbon atoms are optionally substituted with oxo or C₁-C₅ alkyl, and wherein one or more carbon atoms are optionally replaced by a 3-6-membered bridged cycloalkyl, alkenyl, O, NR₈, 3-6 membered cycloalkyl, or 3-6-membered heterocycle, wherein the 3-6-membered heterocycle, the 3-6 membered cycloalkyl, or the 3-6 membered bridged cycloalkyl are optionally substituted with 1, 2, 3, or 4 R₈;
   wherein R₈ is hydrogen or C₁-C₅ alkyl; and
   wherein each R₉ is independently selected from hydrogen, oxo, and C₁-C₅ alkyl; with the proviso that the compound of Formula (II) is not (1¹S,1³R,Z)-2¹H-12-oxa-3,6,10-triaza-4(4,2)-pyridina-2(3,5)-pyrazola-1(1,3)-cyclopentanacyclododecaphane-5,11-dione; (1¹S,1³R,Z)-2¹H-12-oxa-3,6,10-triaza-4(3,5)-pyridina-2(5,3)-pyrazola-1(1,3)-cyclopentanacyclododecaphane-5,11-dione; (1¹S,1³R,Z)-2¹H-12-oxa-3,6,10-triaza-2(5,3)-pyrazola-4(1,3)-benzena-1 (1,3)-cyclopentanacyclododecaphane-5,11-dione; or (1¹S,1³R,Z)-2¹H-13-oxa-3,6,11-triaza-2(5,3)-pyrazola-4(1,3)-benzena-1(1,3)-cyclopentanacyclotridecaphane-5,12-dione.
      2. A compound of Formula (III), or a tautomer, a stereoisomer or a mixture of stereoisomers, a pharmaceutically acceptable salt, a hydrate, or a deuterated derivative thereof:

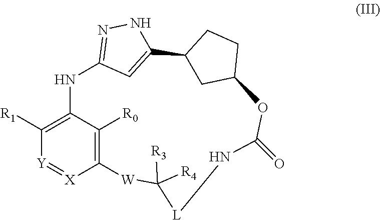

wherein R₀, R₁, X, Y, W, R₃, R₄, and L are as defined in clause 1.
   3. A compound of Formula (VI), or a tautomer, a stereoisomer or a mixture of stereoisomers, a pharmaceutically acceptable salt, a hydrate, or a deuterated derivative thereof:

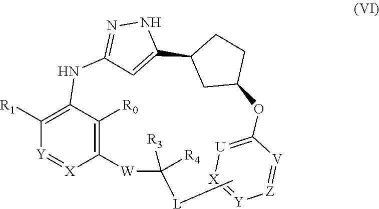

wherein R₀, R₁, X, Y, W, R₃, R₄, and L are as defined in clause 1; and
wherein:
   U, V, and Z are independently selected from N and CR_{A1}.

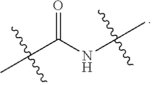 [SMILES: *NC(*)=O]

4. The compound according to clause 1, wherein A is
5. The compound according to clause 1 or 3, wherein A is selected from a 5-membered heterocyclyl and a 6-membered heterocyclyl.
6. The compound according to clause 5, wherein A is independently selected from

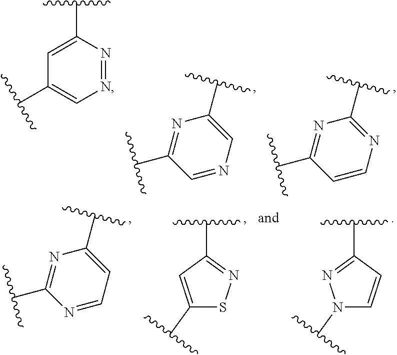

7. The compound according to clause 1 to 3, wherein R_{A1} is C₁-C₅ alkoxide.
8. The compound according to clause 1 to 3, wherein R_{A2} is C₁-C₅ alkoxide.
9. The compound according to any one of clauses 1 to 8, wherein R₀ is hydrogen.
10. The compound according to any one of clauses 1 to 8, wherein R₀ is halo.
11. The compound according to any one of clauses 1 to 8, wherein R₀ is —CN.
12. The compound according to any one of clauses 1 to 8, wherein R₀ is C₁-C₅ alkyl.
13. The compound according to any one of clauses 1 to 12, wherein R₁ is hydrogen.
14. The compound according to any one of clauses 1 to 12, wherein R₁ is halo.
15. The compound according to clause 14, where R₁ is selected from F, Cl, I, and Br.
16. The compound according to clause 12, wherein R₁ is C₁-C₅ alkyl.
17. The compound according to clause 16, wherein R₁ is selected from —CH₃, —CH₂—CH₃, —CH(CH₃)₂, and —CH₂—CH₂—CH₃.
18. The compound according to any one of clauses 1 to 17, wherein X is N.
19. The compound according to any one of clauses 1 to 17, wherein Y is N.
20. The compound according to any one of clauses 1 to 17, wherein X is CR₂.
21. The compound according to any one of clauses 1 to 17, wherein Y is CR₂.
22. The compound according to any one of clause 1 to 17 wherein X and Y are each CR₂.
23. The compound according to any one of clauses 20 to 22, wherein R₂ is selected from hydrogen, haloalkyl, C₁-C₅ alkyl, C₁-C₅ alkoxy, C₁-C₅ alkoxide, and C₃-C₅ cycloalkyl.
24. The compound according to clause 23, wherein R₂ is selected from hydrogen,

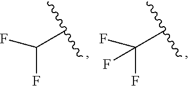

—CH₃, —CH₂—CH₃, —CH(CH₃)₂, —CH₂—CH₂—CH₃,

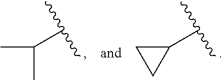

25. The compound according to clause 24, wherein R₂ is hydrogen,

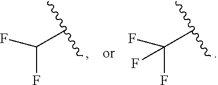

26. The compound according to clause 23, wherein R₂ is C₁-C₅ alkoxide.
27. The compound according to clause 1, wherein Y and R₁ together with the atoms to which they are attached form a 5-6 membered heterocycle or 5-6 membered carbocycle.
28. The compound according to any one of clauses 1 to 27, wherein R₃ and R₄ are independently absent.
29. The compound according to any one of clauses 1 to 27, wherein R₃ and R₄ are independently selected from hydrogen, C₁-C₅ alkyl, and halo.
30. The compound according to any one of clauses 1 to 27, wherein R₃ and R₄ together with the carbon atom to which they are attached, form a carbonyl group, or a 3-6 membered cycloalkyl.
31. The compound according to any one of clauses 1 to 30, wherein W is absent.
32. The compound according to clauses 1 to 30, wherein W is selected from alkylene, —C(O)—R₆—, —O—, —R₆—O—, —O—R₆—, —N(R₇)—, —NH—C(═O)—; —C(═O)—N(R₆)—, —S(═O)₂—, —P(R₇)₂—, 3-6 membered cycloalkyl, and 3-6 membered heterocycle, wherein the alkylene, 3-6 membered cycloalkyl, or 3-6 membered heterocycle are optionally substituted with 1, 2, 3, 4, or 5 R_{B}.
33. The compound according to clause 32, wherein R₆ is absent.
34. The compound according to clause 32, wherein R₆ is independently selected from

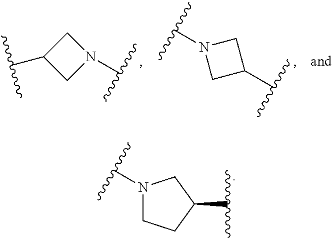 [SMILES: *C1CN(*)C1 ; *C1CN(*)C1 ; *[C@H]1CCN(*)C1]

35. The compound according to clause 32, wherein R₆ is independently selected from —CH₂—, —CH₂—CH₂—, —CH₂—CH₂—CH₂—, and —CH₂—CH₂—CH₂—CH₂—.
36. The compound according to clause 32, wherein W is a 3-6 membered cycloalkyl or a 3-6 membered heterocycle optionally substituted with 1, 2, 3, or 4 R_{B}.
37. The compound according to clause 36, wherein each R_{B} is independently selected from hydrogen, oxo, —CH₃, —CH₂—CH₃, —CH(CH₃)₂, and —CH₂—CH₂—CH₃.
38. A compound according to clause 36, wherein W is selected from

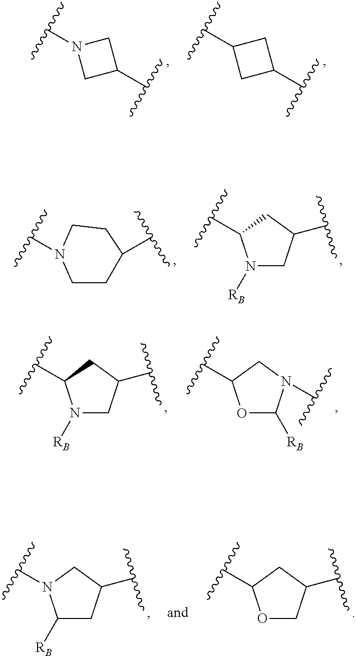

39. The compound according to any one of clauses 1 to 38, wherein L is absent.
40. The compound according to any one of clauses 1 to 38, wherein L is a linker of 1 to 5 carbon atoms in length, wherein one or more carbon atoms are optionally substituted with oxo or C₁-C₅ alkyl.
41. The compound according to clause 40, wherein one or more carbon atoms are optionally substituted with C₁-C₅ alkyl, wherein the C₁-C₅ alkyl is independently selected from —CH₃, —CH₂—CH₃, —CH(CH₃)₂, and —CH₂—CH₂—CH₃.
42. The compound according to any one of clauses 1 to 38, 40, and 41, wherein L is a linker of 1 to 5 carbon atoms in length, wherein one or more carbon atoms are optionally replaced by a 3-6-membered bridged cycloalkyl, alkenyl, —O—, —N(R₈)—, 3-6-membered cycloalkyl, or 3-6-membered heterocycle, and wherein the 3-6-membered heterocycle, the 3-6-membered cycloalkyl, or the 3-6 membered bridged cycloalkyl are optionally substituted with 1, 2, 3, or 4 R₈.
43. The compound according to clause 42, wherein R₈ is selected from hydrogen, —CH₃, —CH₂—CH₃, —CH(CH₃)₂, and —CH₂—CH₂—CH₃.
44. The compound according to clause 42, wherein each R₉ is independently selected from hydrogen, oxo, —CH₃, —CH₂—CH₃, —CH(CH₃)₂, and —CH₂—CH₂—CH₃.
45. The compound according to clause 42, wherein L is a linker of 1 to 5 carbon atoms in length, and wherein the one or more carbon atoms are optionally replaced by a 3-6 membered bridged cycloalkyl, a 3-6 membered cycloalkyl, or a 3-6 membered heterocycle.
46. The compound according to clause 45, wherein the 3-6 membered bridged cycloalkyl, 3-6 membered cycloalkyl, and the 3-6 membered heterocycle are independently selected from

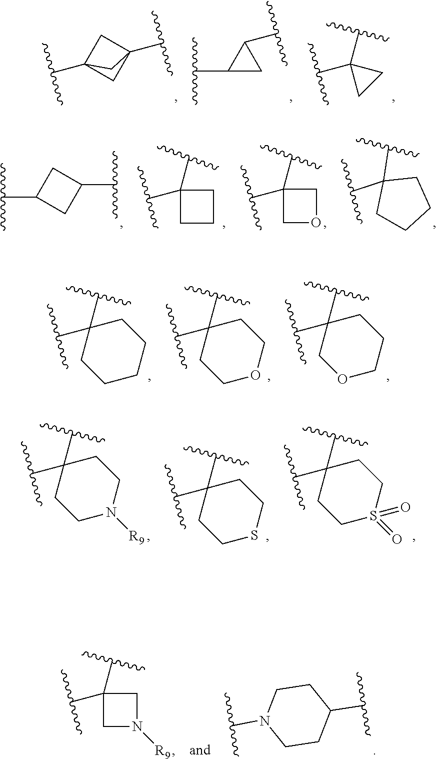

47. The compound according to any one of clauses 1 to 38, and 40 to 46, wherein L is selected from

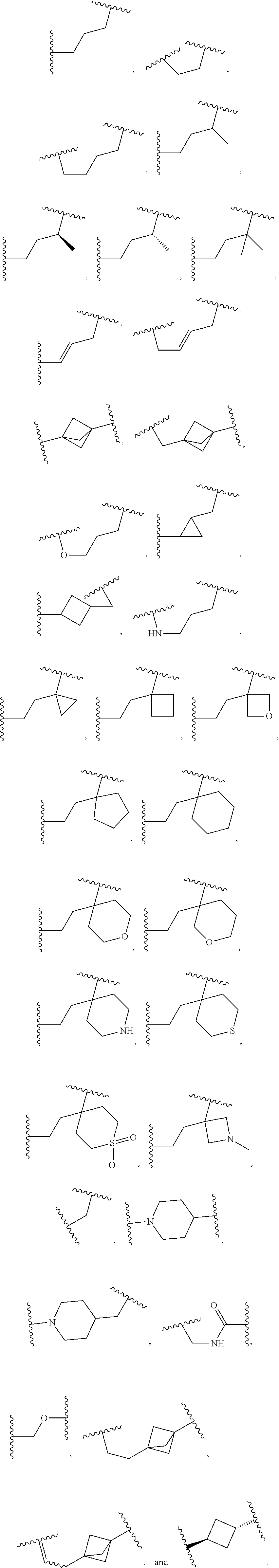

48. A pharmaceutical composition comprising at least one entity chosen from the compounds of any one of clauses 1 to 47, tautomers thereof, stereoisomers thereof, pharmaceutically acceptable salts thereof, hydrates thereof, and deuterated derivatives thereof, and at least one pharmaceutically acceptable carrier or excipient.
49. A method of treating a disease or condition modulated at least in part by CDK2 in a subject, comprising administering to the subject in need thereof, at least one entity chosen from the compounds of any one of clauses 1 to 47, tautomers thereof, stereoisomers thereof, pharmaceutically acceptable salts thereof, hydrates thereof, and deuterated derivatives thereof, or the pharmaceutical composition of clause 48.
50. A method of inhibiting CDK2 in a patient, comprising administering to the patient at least one entity chosen from the compounds of any one of clauses 1 to 47, tautomers thereof, stereoisomers thereof, pharmaceutically acceptable salts thereof, hydrates thereof, and deuterated derivatives thereof, or the pharmaceutical composition of clause 48.
51. A method of treating a disease or disorder dissociated with CDK2 in a patient, comprising administering to the patient at least one entity chosen from the compounds of any one of clauses 1 to 47, tautomers thereof, stereoisomers thereof, pharmaceutically acceptable salts thereof, hydrates thereof, and deuterated derivatives thereof, or the pharmaceutical composition of clause 48; wherein the disease or disorder is associated with an amplification of the cyclin E1 (CCNE1) gene and/or overexpression of CCNE1.
52. The method of any one of clauses 49 to 51, wherein the disease or disorder is cancer.
53. The method of clause 52, wherein the cancer is independently selected from breast cancer, ovarian cancer, uterine cancer, endometrial cancer, cervical cancer, bladder cancer, prostate cancer, lung cancer, stomach cancer, esophageal cancer, colorectal cancer, small bowel cancer, pancreatic cancer, liver cancer, kidney cancer, head and neck cancer, skin cancer, bone cancer, thyroid cancer, peritoneal cancer, and brain cancer.

1. At least one entity selected from compounds of Formula (I), tautomers, stereoisomers or mixture of stereoisomers, pharmaceutically acceptable salts, hydrates, and deuterated derivatives thereof: 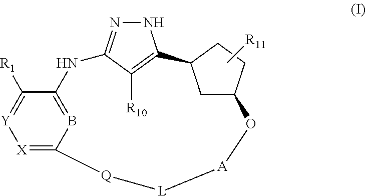
wherein:
A is a substituted or unsubstituted amide or a 5-12 membered aromatic ring, wherein the 5-12 membered aromatic ring optionally includes one or more heteroatoms and is optionally substituted with 1, 2, 3, or 4 R_{A1}, wherein each R_{A1} is independently selected from hydrogen, —C(═O)R_{A2}, C₁-C₅ alkyl, C₂-C₅ alkenyl, C₂-C₅ alkynyl, halo, C₁-C₅ haloalkyl, C₁-C₅ alkoxy, and —CN, and wherein each R_{A2} is independently selected from hydrogen, hydroxyl, C₁-C₅ alkyl, C₁-C₅ alkoxy, C₁-C₅ haloalkyl, C₂-C₅ alkenyl, and C₂-C₅ alkynyl;
B is selected from N, N⁺—O⁻, and CR₀, wherein R₀ is selected from hydrogen, halo, —CN, C₁-C₅ alkyl, C₁-C₅ haloalkyl, C₁-C₅ alkoxy, and C₃-C₅ cycloalkyl;
R₁ is selected from hydrogen, halo, —CN, and C₁-C₅ alkyl, or R₁ and Y together with the carbon atom to which they are attached form a 5-6 membered aromatic ring, wherein the aromatic ring optionally includes 1 to 3 heteroatoms;
X and Y are independently N, N⁺—O⁻, or CR₂, wherein each R₂ is independently selected from hydrogen, haloalkyl, C₁-C₅ alkyl, C₁-C₅ alkoxy, and C₃-C₅ cycloalkyl;
Q is absent or selected from —W—C(R₃)(R₄)—, wherein R₃ and R₄ are independently selected from hydrogen, deuterium, C₁-C₅ alkyl, C₁-C₅ haloalkyl, and halo, or R₃ and R₄ together with the carbon atom to which they are attached form a carbonyl group or a 3-6 membered cycloalkyl, and wherein W is absent or selected from alkylene, —C(═O)—R₆—, —O—, —R₆—O—, —O—R₆—, —N(R₇)—, —NH—C(═O)—, —C(═O)—N(R₆)—, —S—, —S(═O)₂—, —P(R₇)₂—, 3-6 membered cycloalkyl, and 3-6 membered heterocycle, wherein the alkylene, 3-6 membered cycloalkyl, and 3-6 membered heterocycle are optionally substituted with 1, 2, 3, 4, or 5 R_{B}, wherein each R₆ is independently selected from C₁-C₅ alkylene, 3-6 membered heterocycle, and 3-6 membered cycloalkyl, wherein each R₇ is independently selected from hydrogen, oxo, and C₁-C₅ alkyl, and wherein each R_{B} is independently selected from hydrogen, oxo, halo, and C₁-C₅ alkyl;
L is absent or a linker of 1 to 10 carbon atoms in length, wherein one or more carbon atoms are optionally substituted with oxo, C₁-C₅ haloalkyl, halo, deuterium, or C₁-C₅ alkyl, and wherein one or more carbon atoms are optionally replaced by a 3-6-membered bridged cycloalkyl, a 3-7 membered fused cycloalkyl, alkenyl, —O—, —N(R₈)—, a 3-6-membered cycloalkyl, or a 3-6-membered heterocycle, wherein the 3-6-membered heterocycle, the 3-6-membered cycloalkyl, and the 3-6-membered bridged cycloalkyl are optionally substituted with 1, 2, 3, or 4 R₉, wherein each R₈ is independently selected from hydrogen and C₁-C₅ alkyl, and wherein each R₉ is independently selected from hydrogen, oxo, and C₁-C₅ alkyl; and
R₁₀ and R₁₁ are independently selected from hydrogen, halo, and C₁-C₅ alkyl;
with the proviso that the compound of Formula (I) is not the following:
(1¹S,1³R,Z)-2¹H-12-oxa-3,6,10-triaza-4(4,2)-pyridina-2(3,5)-pyrazola-1(1,3)-cyclopentanacyclododecaphane-5,11-dione,
(1¹S,1³R,Z)-2¹H-12-oxa-3,6,10-triaza-4(3,5)-pyridina-2(5,3)-pyrazola-1(1,3)-cyclopentanacyclododecaphane-5,11-dione,
(1¹S,1³R,Z)-2¹H-12-oxa-3,6,10-triaza-2(5,3)-pyrazola-4(1,3)-benzena-1 (1,3)-cyclopentanacyclododecaphane-5,11-dione, or
(1¹S,1³R,Z)-2¹H-13-oxa-3,6,11-triaza-2(5,3)-pyrazola-4(1,3)-benzena-1 (1,3)-cyclopentanacyclotridecaphane-5,12-dione.

2. The at least one entity according to claim 1, wherein the at least one entity is selected from compounds of Formula (II), tautomers, stereoisomers or mixture of stereoisomers, pharmaceutically acceptable salts, hydrates, and deuterated derivatives thereof:

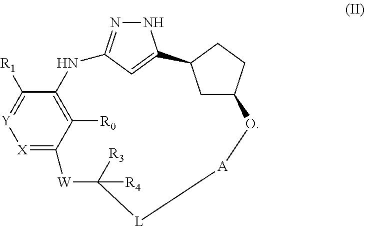

3. The at least one entity according to claim 1, wherein the at least one entity is selected from compounds of Formula (III), tautomers, stereoisomers or mixture of stereoisomers, pharmaceutically acceptable salts, hydrates, and deuterated derivatives thereof:

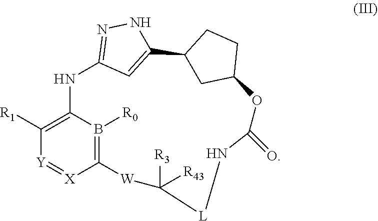

4. The at least one entity according to claim 1, wherein the at least one entity is selected from compounds of Formula (IV), tautomers, stereoisomers or mixture of stereoisomers, pharmaceutically acceptable salts, hydrates, and deuterated derivatives thereof:

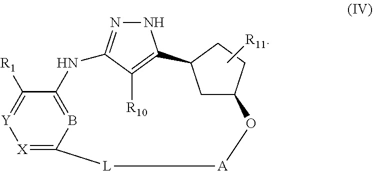

5. The at least one entity according to claim 1, wherein the at least one entity is selected from compounds of Formula (V), tautomers, stereoisomers or a mixture of stereoisomers, pharmaceutically acceptable salts, hydrates, and deuterated derivatives thereof:

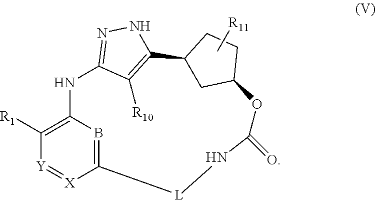

6. The at least one entity according to claim 1, wherein A is

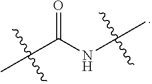 [SMILES: *NC(*)=O]

7. The at least one entity according to claim 1, wherein A is selected from a 5-membered aromatic ring and a 6-membered aromatic ring, wherein the 5-membered aromatic ring and the 6-membered aromatic ring optionally include one or more heteroatoms.

8. The at least one entity according to claim 7, wherein the 5-membered aromatic ring and 6-membered aromatic ring include one or more heteroatoms.

9. The at least one entity according to claim 8, wherein A is selected from

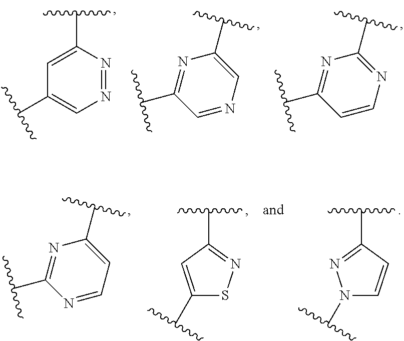

10. The at least one entity according to claim 1, wherein at least one R_{A1} is C₁-C₅ alkoxy.

11. The at least one entity according to claim 1, wherein at least one R_{A1} is hydrogen.

12. The at least one entity according to claim 1, wherein at least one R_{A1} is C₁-C₅ alkyl.

13. The at least one entity according to claim 1, wherein at least one R_{A1} is halo.

14. The at least one entity according to claim 1, wherein at least one R_{A1} is C₁-C₅ haloalkyl.

15. The at least one entity according to claim 1, wherein at least one R_{A1} is —CN.

16. The at least one entity according to claim 1, wherein at least one R_{A1} is —C(═O)—R_{A2}.

17. The at least one entity according to claim 16, wherein R_{A2} is C₁-C₅ alkoxy.

18. The at least one entity according to claim 16, wherein R_{A2} is C₁-C₅ alkyl.

19. The at least one entity according to claim 16, wherein R_{A2} is hydrogen.

20. The at least one entity according to claim 16, wherein R_{A2} is hydroxyl.

21. The at least one entity according to claim 16, wherein R_{A2} is C₁-C₅ haloalkyl.

22. The at least one entity according to claim 16, wherein R_{A2} is C₂-C₅ alkynyl.

23. The at least one entity according to claim 1, wherein B is N or N⁺—O⁻.

24. The at least one entity according to claim 1, wherein B is CR₀

25. The at least one entity according to claim 24, wherein R₀ is hydrogen.

26. The at least one entity according to claim 24, wherein R₀ is halo.

27. The at least one entity according to claim 24, wherein R₀ is —CN.

28. The at least one entity according to claim 24, wherein R₀ is C₁-C₅ alkyl.

29. The at least one entity according to claim 24, wherein R₀ is C₁-C₅ haloalkyl.

30. The at least one entity according to claim 24, wherein R₀ is C₁-C₅ alkoxy.

31. The at least one entity according to claim 24, wherein R₀ is and C₁-C₅ cycloalkyl.

32. The at least one entity according to claim 1, wherein R₁ is hydrogen.

33. The at least one entity according to claim 1, wherein R₁ is halo.

34. The at least one entity according to claim 33, wherein R₁ is independently selected from F, Cl, I, and Br.

35. The at least one entity according to claim 1, wherein R₁ is C₁-C₅ alkyl.

36. The at least one entity according to claim 35, wherein R₁ is selected from —CH₃, —CH₂—CH₃, —CH(CH₃)₂, and —CH₂—CH₂—CH₃.

37. The at least one entity according to claim 1, wherein R₁ and Y together with the carbon atom to which they are attached form a 5-6 membered aromatic ring, wherein the 5-6 membered aromatic ring optionally includes one or more heteroatoms.

38. The at least one entity according to claim 37, wherein the 5-6 membered aromatic ring includes 1 to 3 heteroatoms.

39. The at least one entity according to claim 1, wherein Y is N or N⁺—O⁻.

40. The at least one entity according to claim 1, wherein Y is CR₂.

41. The at least one entity according to claim 1, wherein X is N or N⁺—O⁻.

42. The at least one entity according to claim 41, wherein X is N.

43. The at least one entity according to claim 1, wherein X is CR₂.

44. The at least one entity according to claim 40, wherein R₂ is independently selected from hydrogen, halo, haloalkyl, C₁-C₅ alkyl, C₁-C₅ alkoxy, and C₃-C₅ cycloalkyl.

45. The at least one entity according to claim 44, wherein at least one R₂ is independently selected from hydrogen, —CH₃, —CH₂—CH₃, —CH(CH₃)₂, —CH₂—CH₂—CH₃,

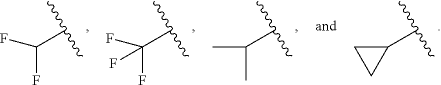

46. The at least one entity according to claim 44, wherein at least one R₂ is hydrogen,

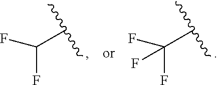

47. The at least one entity according to claim 44, wherein at least one R₂ is C₁-C₅ alkoxy.

48. The at least one entity according to claim 44, wherein at least one R₂ is halo selected from F, Cl, Br, and I.

49. The at least one entity according to claim 1, wherein Q is absent.

50. The at least one entity according to claim 1, wherein Q is —W—C(R₃)(R₄)—.

51. The at least one entity according to claim 50, wherein R₃ and R₄ are independently selected from hydrogen, deuterium, C₁-C₅ alkyl, C₁-C₅ haloalkyl, and halo.

52. The at least one entity according to claim 50, wherein at least one of R₃ or R₄ is hydrogen.

53. The at least one entity according to claim 50, wherein R₃ and R₄ together with the carbon atom to which they are attached form a carbonyl group or a 3-6 membered cycloalkyl.

54. The at least one entity according to claim 50, wherein W is absent.

55. The at least one entity according to claim 50, wherein W is independently selected from alkylene, —C(═O)—R₆—, —O—, —R₆—O—, —O—R₆—, —N(R₇)—, —NH—C(═O)—, —C(═O)—N(R₆)—, —S(═O)₂—, —P(R₇)₂—, 3-6 membered cycloalkyl, and 3-6 membered heterocycle, wherein the alkylene, 3-6 membered cycloalkyl, and 3-6 membered heterocycle are optionally substituted with 1, 2, 3, 4, or 5 R_{B}.

56. The at least one entity according to claim 55, wherein R₆ is selected from

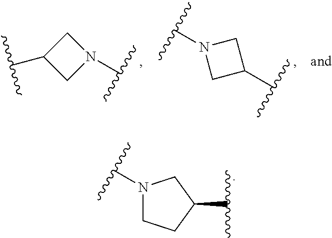 [SMILES: *C1CN(*)C1 ; *C1CN(*)C1 ; *[C@H]1CCN(*)C1]

57. The at least one entity according to claim 55, wherein R₆ is selected from —CH₂—, —CH₂—CH₂—, —CH₂—CH₂—CH₂—, and —CH₂—CH₂—CH₂—CH₂—.

58. The at least one entity according to claim 55, wherein W is a 3-6 membered cycloalkyl or a 3-6 membered heterocycle, wherein the 3-6 membered cycloalkyl and 3-6 membered heterocycle are optionally substituted with 1, 2, 3, or 4 R_{B}.

59. The at least one entity according to claim 58, wherein W is a 3-6 membered cycloalkyl or a 3-6 membered heterocycle, wherein the 3-6 membered cycloalkyl and 3-6 membered heterocycle are substituted with 1, 2, 3, or 4 R_{B}.

60. The at least one entity according to claim 55, wherein W is selected from

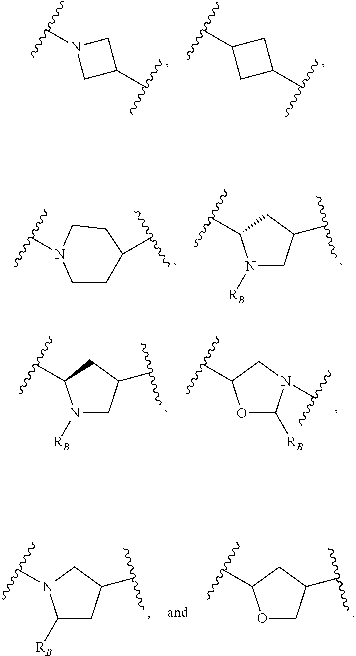

61. The at least one entity according to claim 55, wherein at least one R_{B} is independently selected from hydrogen, oxo, —CH₃, —CH₂—CH₃, —CH(CH₃)₂, and —CH₂—CH₂—CH₃.

62. The at least one entity according to claim 1, wherein L is absent.

63. The at least one entity according to claim 1, wherein L is a linker of 1 to 10 carbon atoms in length, wherein one or more carbon atoms of L are optionally substituted with oxo, C₁-C₅ haloalkyl, halo, deuterium, or C₁-C₅ alkyl, and wherein one or more carbon atoms of L are optionally replaced by a 3-6-membered bridged cycloalkyl, a 3-7 membered fused cycloalkyl, alkenyl, —O—, —S—, —S(═O)₂—, —N(R₈), 3-6-membered cycloalkyl, or 3-6-membered heterocycle, wherein the 3-6-membered heterocycle, the 3-6-membered cycloalkyl, and the 3-6 membered bridged cycloalkyl are optionally substituted with 1, 2, 3, or 4 R₉.

64. The at least one entity according to claim 63, wherein L is a linker of 1 to 5 carbon atoms in length.

65. The at least one entity according to claim 63, wherein one or more carbon atoms of L are substituted with oxo, C₁-C₅ haloalkyl, halo, deuterium, or C₁-C₅ alkyl.

66. The at least one entity according to claim 63, wherein one or more carbon atoms of L are substituted with oxo, C₁-C₅ haloalkyl, halo, or C₁-C₅ alkyl.

67. The at least one entity according to claim 63, wherein one or more carbon atoms of L are optionally substituted with C₁-C₅ alkyl.

68. The at least one entity according to claim 67, wherein one or more carbon atoms of L are optionally substituted with —CH₃, —CH₂—CH₃, —CH(CH₃)₂, or —CH₂—CH₂—CH₃.

69. The at least one entity according to claim 65, wherein one or more carbon atoms of L are optionally substituted with deuterium.

70. The at least one entity according to claim 63, wherein one or more carbon atoms of L are replaced by a 3-6-membered bridged cycloalkyl, a 3-7 membered fused cycloalkyl, alkenyl, —O—, —S—, —S(═O)₂—, —N(R₈), 3-6-membered cycloalkyl, or 3-6-membered heterocycle, wherein the 3-6-membered heterocycle, the 3-6-membered cycloalkyl, and the 3-6 membered bridged cycloalkyl are optionally substituted with 1, 2, 3, or 4 R₉.

71. The at least one entity according to claim 70, wherein at least one R₈ is independently selected from hydrogen, —CH₃, —CH₂—CH₃, —CH(CH₃)₂, and —CH₂—CH₂—CH₃.

72. The at least one entity according to claim 70, wherein the 3-6-membered heterocycle, the 3-6-membered cycloalkyl, and the 3-6 membered bridged cycloalkyl are substituted with 1, 2, 3, or 4 R₉.

73. The at least one entity according to claim 72, wherein at least one R₉ is independently selected from hydrogen, halo, oxo, —CH₃, —CH₂—CH₃, —CH(CH₃)₂, and —CH₂—CH₂—CH₃.

74. The at least one entity according to claim 70, wherein the 3-6 membered bridged cycloalkyl, 3-6 membered cycloalkyl, and the 3-6 membered heterocycle are independently selected from:

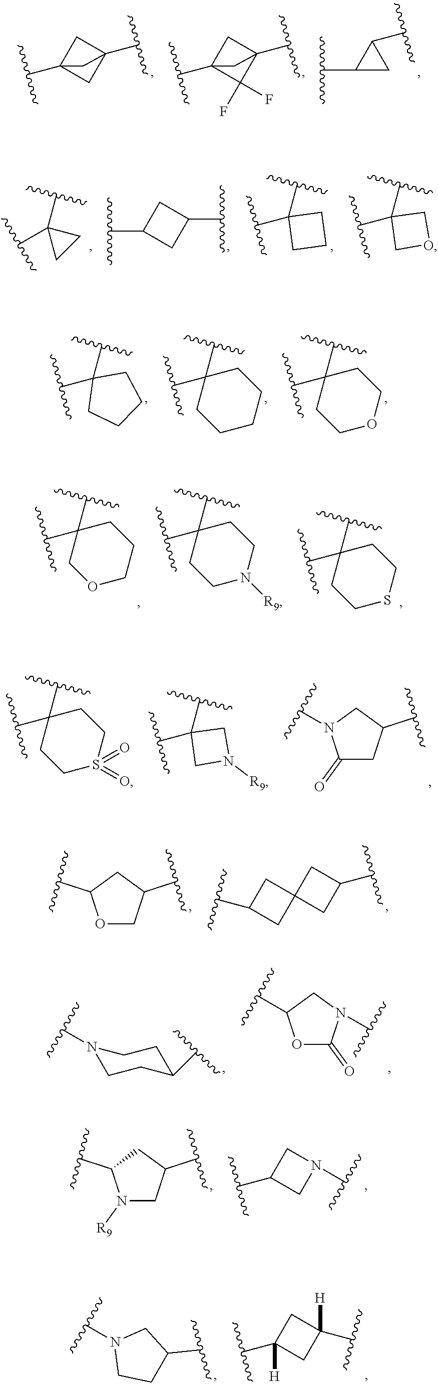

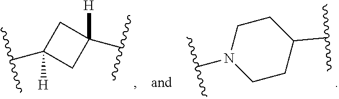 [SMILES: *[C@H]1C[C@H](*)C1 ; *C1CCN(*)CC1]

75. The at least one entity according to claim 1, wherein L is selected from

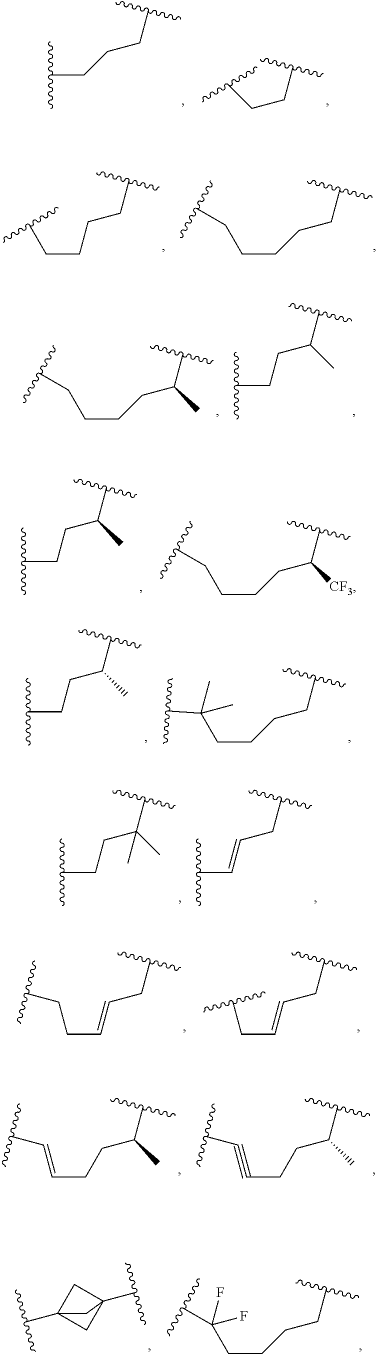

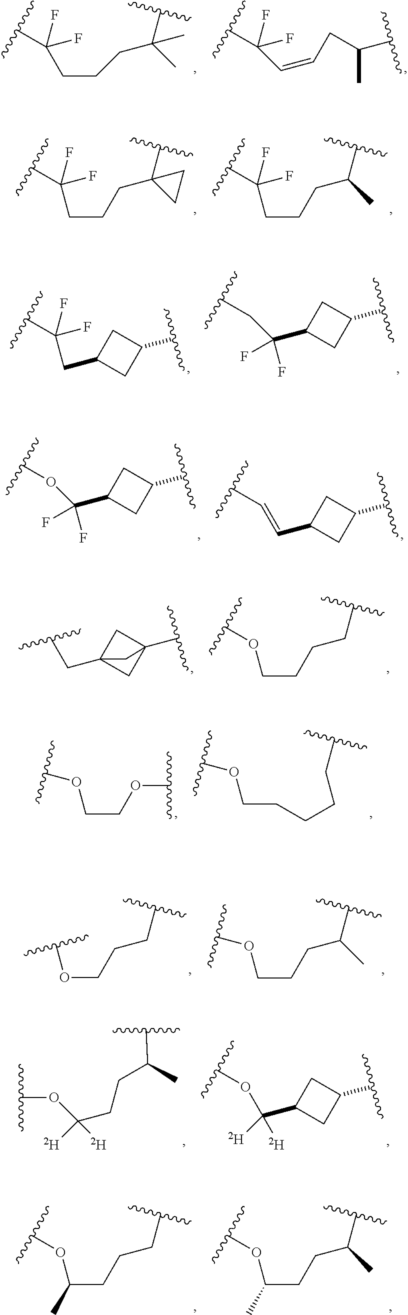

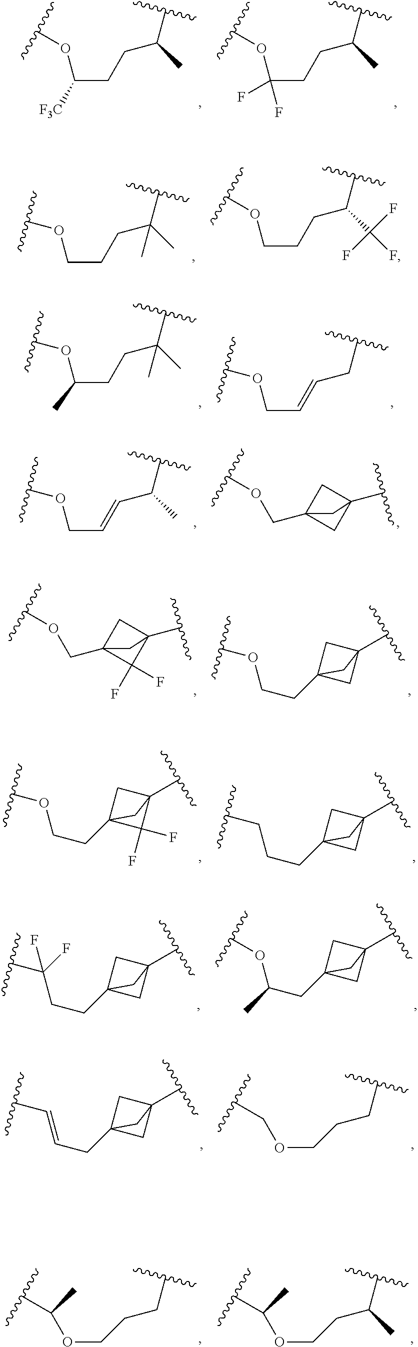

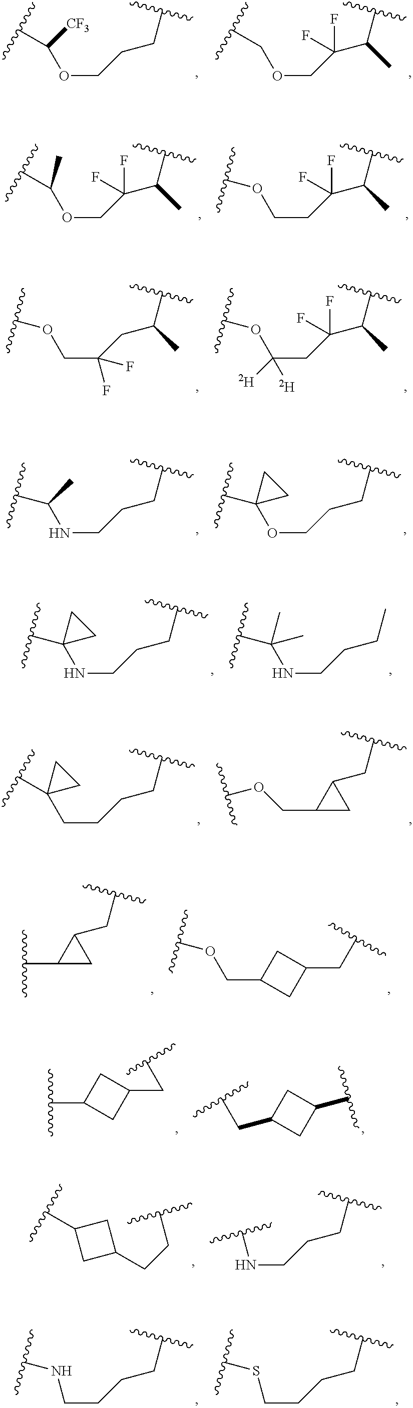

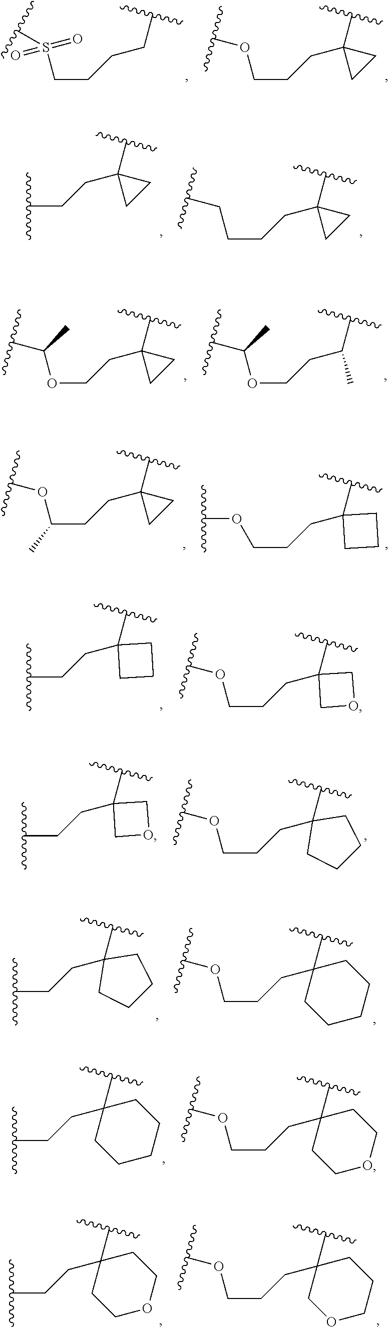

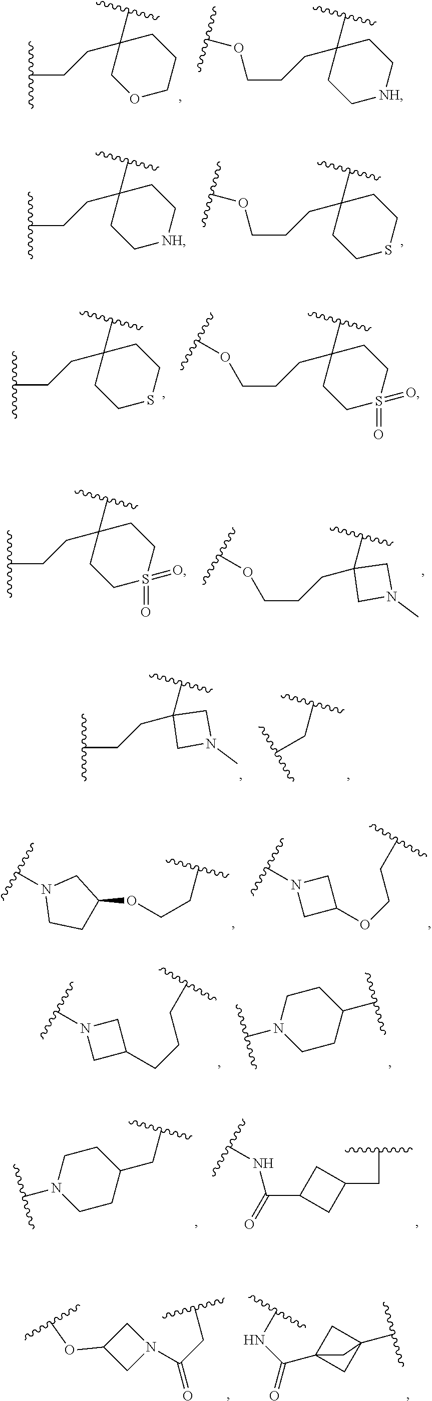

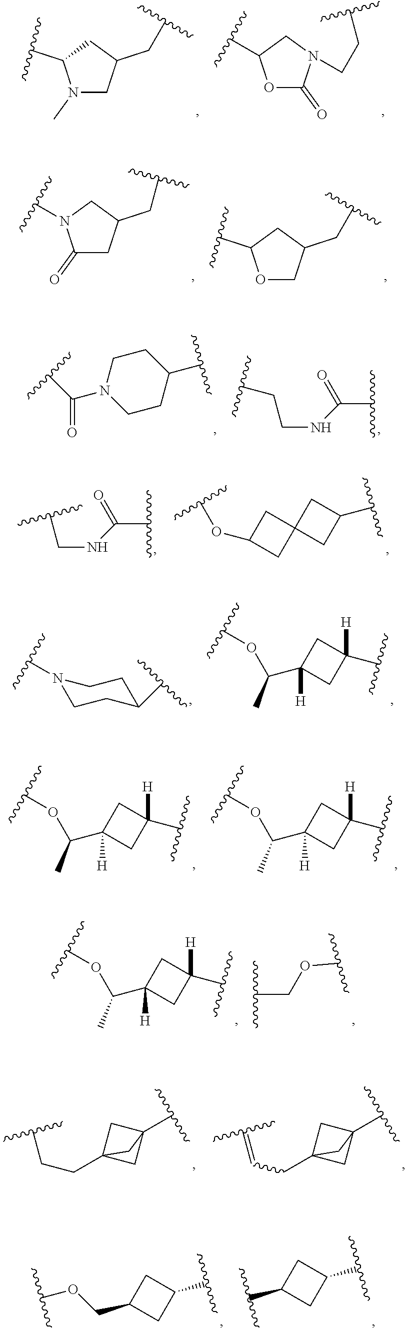

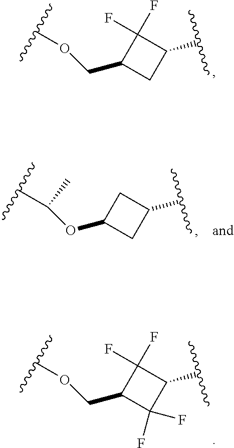

76. The at least one entity according to claim 1, wherein the at least one entity is selected from:
(1¹S,1³R,9S,Z)-9-methyl-2¹H-5,12-dioxa-3,10-diaza-4(4,2)-pyridina-2(3,5)-pyrazola-1(1,3)-cyclopentanacyclododecaphan-11-one,
(1¹S,1³R,9R,Z)-9-methyl-2¹H-5,12-dioxa-3,10-diaza-4(4,2)-pyridina-2(3,5)-pyrazola-1(1,3)-cyclopentanacyclododecaphan-11-one,
(1¹S,1³R,7¹R,7³S,Z)-2¹H-5,10-dioxa-3,8-diaza-4(4,2)-pyridina-2(3,5)-pyrazola-1(1,3)-cyclopentana-7(1,3)-cyclobutanacyclodecaphan-9-one,
(1'S,3′R,6′R,Z)-6′-methylspiro[cyclopropane-1 9′-5,12-dioxa-3,10-diaza-4(4,2)-pyridina-2(3,5)-pyrazola-1 (1,3)-cyclopentanacyclododecaphan]-11′-one,
(1'S,3′R,6'S,Z)-6′-methylspiro[cyclopropane-1,9′-5,12-dioxa-3,10-diaza-4(4,2)-pyridina-2(3,5)-pyrazola-1 (1,3)-cyclopentanacyclododecaphan]-11′-one,
(1¹S,1³R,7¹S,7³S,6R,Z)-6-methyl-2¹H-5,10-dioxa-3,8-diaza-4(4,2)-pyridina-2(3,5)-pyrazola-1 (1,3)-cyclopentana-7(1,3)-cyclobutanacyclodecaphan-9-one,
(1¹S,1³R,7¹R,7³R,6R,Z)-6-methyl-2¹H-5,10-dioxa-3,8-diaza-4(4,2)-pyridina-2(3,5)-pyrazola-1(1,3)-cyclopentana-7(1,3)-cyclobutanacyclodecaphan-9-one,
(1¹S,1³R,7¹S,7³R,6S,Z)-6-methyl-2¹H-5,10-dioxa-3,8-diaza-4(4,2)-pyridina-2(3,5)-pyrazola-1 (1,3)-cyclopentana-7(1,3)-cyclobutanacyclodecaphan-9-one,
(1¹S,1³R,7¹R,7³S,6S,Z)-6-methyl-2¹H-5,10-dioxa-3,8-diaza-4(4,2)-pyridina-2(3,5)-pyrazola-1 (1,3)-cyclopentana-7(1,3)-cyclobutanacyclodecaphan-9-one,
(1¹S,1³R,6S,9S,Z)-6,9-dimethyl-2¹H-5,12-dioxa-3,10-diaza-4(4,2)-pyridina-2(3,5)-pyrazola-1(1,3)-cyclopentanacyclododecaphan-11-one,
(1¹S,1³R,6S,9R,Z)-6,9-dimethyl-2¹H-5,12-dioxa-3,10-diaza-4(4,2)-pyridina-2(3,5)-pyrazola-1(1,3)-cyclopentanacyclododecaphan-11-one,
(1¹S,1³R,9S,Z)-5,5-difluoro-9-methyl-2¹H-12-oxa-3,10-diaza-4(4,2)-pyridina-2(3,5)-pyrazola-1 (1,3)-cyclopentanacyclododecaphan-11-one,
(1¹S,1³R,9S,Z)-8,8-difluoro-9-methyl-2¹H-5,12-dioxa-3,10-diaza-4(4,2)-pyridina-2(3,5)-pyrazola-1 (1,3)-cyclopentanacyclododecaphan-11-one,
(1¹S,1³R,9S,Z)-8,8-difluoro-9-methyl-2¹H-5,12-dioxa-3,10-diaza-4(4,2)-pyridina-2(3,5)-pyrazola-1 (1,3)-cyclopentanacyclododecaphan-11-one-6,6-d₂,
(1¹S,1³R,9S,E)-2⁴-fluoro-9-methyl-2¹H-5,12-dioxa-3,10-diaza-4(4,2)-pyridina-2(3,5)-pyrazola-1(1,3)-cyclopentanacyclododecaphan-11-one,
(1¹S,1³R,9S,E)-2⁴,8,8-trifluoro-9-methyl-2¹H-5,12-dioxa-3,10-diaza-4(4,2)-pyridina-2(3,5)-pyrazola-1 (1,3)-cyclopentanacyclododecaphan-11-one,
(1¹S,1³R,9S,Z)-7,7-difluoro-9-methyl-2¹H-5,12-dioxa-3,10-diaza-4(4,2)-pyridina-2(3,5)-pyrazola-1 (1,3)-cyclopentanacyclododecaphan-11-one,
(1¹R,1³S,1⁴R,9S,Z)-1⁴-fluoro-9-methyl-2¹H-5,12-dioxa-3,10-diaza-4(4,2)-pyridina-2(3,5)-pyrazola-1 (1,3)-cyclopentanacyclododecaphan-11-one,
(1¹S,1³R,1⁴S,9S,Z)-1⁴-fluoro-9-methyl-2¹H-5,12-dioxa-3,10-diaza-4(4,2)-pyridina-2(3,5)-pyrazola-1(1,3)-cyclopentanacyclododecaphan-11-one,
(1¹R,1³S,1⁴R,9S,Z)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one,
(1¹S,1³R,1⁴S,9S,Z)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one,
(1¹R,1³S,1⁴R,7¹S,7³R,E)-1⁴,2⁴-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one,
(1¹S,1³R,1⁴S,7¹R,7³S,E)-1⁴,2⁴-difluoro-2¹H-5,10-dioxa-3,8-diaza-4(4,2)-pyridina-2(3,5)-pyrazola-1(1,3)-cyclopentana-7(1,3)-cyclobutanacyclodecaphan-9-one,
(1¹S,1³R,9S,Z)-9-methyl-2¹H-5,12-dioxa-3,10-diaza-4(2,6)-pyrazina-2(3,5)-pyrazola-1(1,3)-cyclopentanacyclododecaphan-11-one,
(1¹S,1³R,9S,E)-2⁴-fluoro-9-methyl-2¹H-5,12-dioxa-3,10-diaza-4(2,6)-pyrazina-2(3,5)-pyrazola-1(1,3)-cyclopentanacyclododecaphan-11-one,
(1¹S,1³R,7¹R,7³S,Z)-2¹H-5,10-dioxa-3,8-diaza-4(2,6)-pyrazina-2(3,5)-pyrazola-1(1,3)-cyclopentana-7(1,3)-cyclobutanacyclodecaphan-9-one,
(1¹S,1³R,7¹R,73S,E)-2⁴-fluoro-2¹H-5,10-dioxa-3,8-diaza-4(2,6)-pyrazina-2(3,5)-pyrazola-1(1,3)-cyclopentana-7(1,3)-cyclobutanacyclodecaphan-9-one,
(1¹R,1³S,1⁴R,9S,E)-1⁴,2⁴-difluoro-9-methyl-2¹H-5,12-dioxa-3,10-diaza-4(4,2)-pyridina-2(3,5)-pyrazola-1(1,3)-cyclopentanacyclododecaphan-11-one,
(1¹S,1³R,14S,9S,E)-1⁴,2⁴-difluoro-9-methyl-2¹H-5,12-dioxa-3,10-diaza-4(4,2)-pyridina-2(3,5)-pyrazola-1 (1,3)-cyclopentanacyclododecaphan-11-one,
(1¹R,1³S,1⁴R,9S,E)-1⁴,2⁴,8,8-tetrafluoro-9-methyl-2¹H-5,12-dioxa-3,10-diaza-4(4,2)-pyridina-2(3,5)-pyrazola-1(1,3)-cyclopentanacyclododecaphan-11-one,
(1¹S,1³R,1⁴S,9S,E)-1⁴,2⁴,8,8-tetrafluoro-9-methyl-2¹H-5,12-dioxa-3,10-diaza-4(4,2)-pyridina-2(3,5)-pyrazola-1 (1,3)-cyclopentanacyclododecaphan-11-one,
(1¹S,1³R,9S,Z)-9-methyl-2¹H-5,12-dioxa-3,10-diaza-4(4,2)-pyrimidina-2(3,5)-pyrazola-1(1,3)-cyclopentanacyclododecaphan-11-one,
(1¹R,1³S,1⁴R,9S,Z)-1⁴-fluoro-9-methyl-2¹H-5,12-dioxa-3,10-diaza-4(2,6)-pyrazina-2(3,5)-pyrazola-1(1,3)-cyclopentanacyclododecaphan-11-one,
(1¹R,1³S,1⁴R,9S,E)-1⁴,2⁴-difluoro-9-methyl-2¹H-5,12-dioxa-3,10-diaza-4(2,6)-pyrazina-2(3,5)-pyrazola-1 (1,3)-cyclopentanacyclododecaphan-11-one,
(1¹S,1³R,9S,E)-2⁴,7,7-trifluoro-9-methyl-2¹H-5,12-dioxa-3,10-diaza-4(4,2)-pyridina-2(3,5)-pyrazola-1 (1,3)-cyclopentanacyclododecaphan-11-one, and
(1¹S,1³R,9S,E)-2⁴-fluoro-9-methyl-2¹H-5,12-dioxa-3,10-diaza-4(4,2)-pyrimidina-2(3,5)-pyrazola-1 (1,3)-cyclopentanacyclododecaphan-11-one.

77. The at least one entity according to claim 1, wherein the at least one entity is selected from:
(1¹S,1³R,9S,Z)-9-methyl-2¹H-5,12-dioxa-3,10-diaza-4(4,2)-pyridina-2(3,5)-pyrazola-1(1,3)-cyclopentanacyclododecaphan-11-one,
(1¹S,1³R,9R,Z)-9-methyl-2¹H-5,12-dioxa-3,10-diaza-4(4,2)-pyridina-2(3,5)-pyrazola-1(1,3)-cyclopentanacyclododecaphan-11-one,
(1¹S,1³R,7¹R,7³S,6S,Z)-6-methyl-2¹H-5,10-dioxa-3,8-diaza-4(4,2)-pyridina-2(3,5)-pyrazola-1 (1,3)-cyclopentana-7(1,3)-cyclobutanacyclodecaphan-9-one,
(1¹S,1³R,9S,Z)-5,5-difluoro-9-methyl-2¹H-12-oxa-3,10-diaza-4(4,2)-pyridina-2(3,5)-pyrazola-1 (1,3)-cyclopentanacyclododecaphan-11-one,
(1¹S,1³R,9S,Z)-8,8-difluoro-9-methyl-2¹H-5,12-dioxa-3,10-diaza-4(4,2)-pyridina-2(3,5)-pyrazola-1 (1,3)-cyclopentanacyclododecaphan-11-one,
(1¹S,1³R,9S,Z)-8,8-difluoro-9-methyl-2¹H-5,12-dioxa-3,10-diaza-4(4,2)-pyridina-2(3,5)-pyrazola-1 (1,3)-cyclopentanacyclododecaphan-11-one-6,6-d₂,
(1¹S,1³R,9S,E)-2⁴,8,8-trifluoro-9-methyl-2¹H-5,12-dioxa-3,10-diaza-4(4,2)-pyridina-2(3,5)-pyrazola-1 (1,3)-cyclopentanacyclododecaphan-11-one,
(1¹R,1³S,1⁴R,9S,Z)-1⁴,8,8-trifluoro-9-methyl-2¹H-5,12-dioxa-3,10-diaza-4(4,2)-pyridina-2(3,5)-pyrazola-1(1,3)-cyclopentanacyclododecaphan-11-one, and
(1¹R,1³S,1⁴R,9S,E)-1⁴,2⁴,8,8-tetrafluoro-9-methyl-2¹H-5,12-dioxa-3,10-diaza-4(4,2)-pyridina-2(3,5)-pyrazola-1 (1,3)-cyclopentanacyclododecaphan-11-one.

78. A pharmaceutical composition comprising the at least one entity according to claim 1 and at least one pharmaceutically acceptable carrier or excipient.

79. A method of treating a disease or condition modulated at least in part by CDK2 in a subject, the method comprising administering to the subject in need thereof the at least one entity according to claim 1 or the pharmaceutical composition of claim 78.

80. A method of inhibiting CDK2 in a patient, the method comprising administering to the patient the at least one entity according to claim 1 or the pharmaceutical composition of claim 78.

81. A method of treating a disease or disorder associated with CDK2 in a patient, the method comprising administering to the patient the at least one entity according to claim 1 or the pharmaceutical composition of claim 78, wherein the disease or disorder is associated with an amplification of the cyclin E1 (CCNE1) gene and/or overexpression of CCNE1.

82. The method of claim 81, wherein the disease, disorder, or condition is cancer.

83. The method of claim 82, wherein the cancer is selected from breast cancer, ovarian cancer, uterine cancer, endometrial cancer, cervical cancer, bladder cancer, prostate cancer, lung cancer, stomach cancer, esophageal cancer, colorectal cancer, small bowel cancer, pancreatic cancer, liver cancer, kidney cancer, head and neck cancer, skin cancer, bone cancer, thyroid cancer, peritoneal cancer, and brain cancer.
